(19) United States
(12) Patent Application Publication
(10) Pub. No.: US 2025/0089563 A1
(43) Pub. Date: Mar. 13, 2025

(54) LIGHT-EMITTING DEVICE INCLUDING AMINE-CONTAINING COMPOUND, ELECTRONIC APPARATUS INCLUDING THE LIGHT-EMITTING DEVICE, AND THE AMINE-CONTAINING COMPOUND

(71) Applicant: Samsung Display Co., Ltd., Yongin-si (KR)

(72) Inventors: Dongjun Kim, Yongin-si (KR); Hankyu Pak, Yongin-si (KR); Hyunbin Park, Yongin-si (KR); Jinyoung Yun, Yongin-si (KR); Sanghyun Han, Yongin-si (KR)

(21) Appl. No.: 18/811,476

(22) Filed: Aug. 21, 2024

(30) Foreign Application Priority Data
Sep. 5, 2023 (KR) 10-2023-0117429

(51) Int. Cl. H10K 85/60; C07D 233/96; C07D 239/42; C07D 241/20; C07D 251/42; C07F 5/02; H10K 50/16; H10K 50/858

(52) U.S. Cl. CPC H10K 85/658; C07D 233/96; C07D 239/42; C07D 241/20; C07D 251/42; C07F 5/027; H10K 85/633; H10K 85/636; H10K 50/16; H10K 50/858; H10K 85/615; H10K 85/654

(57) ABSTRACT

An amine-containing compound represented by Formula 1, a light-emitting device including the amine-containing compound, and an electronic apparatus including the light-emitting device are provided:

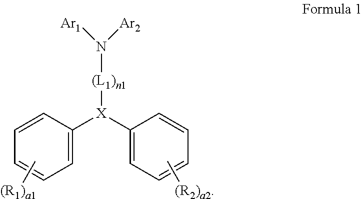

### CROSS-REFERENCE TO RELATED APPLICATION

The present application claims priority to and the benefit of Korean Patent Application No. 10-2023-0117429, filed on Sep. 5, 2023, in the Korean Intellectual Property Office, the entire disclosure of which is incorporated herein by reference.

### BACKGROUND

### 1. Field

One or more embodiments of the present disclosure relate to a light-emitting device including an amine-containing compound, an electronic apparatus including the light-emitting device, and the amine-containing compound.

### 2. Description of the Related Art

Among light-emitting devices, self-emissive devices have relatively wide viewing angles, relatively high contrast ratios, relatively short response times, and excellent or suitable characteristics in terms of luminance, driving voltage, and response speed.

In a light-emitting device, a first electrode is located on a substrate, and a hole transport region, an emission layer, an electron transport region, and a second electrode are sequentially arranged on the first electrode. Holes provided from the first electrode move toward the emission layer through the hole transport region, and electrons provided from the second electrode move toward the emission layer through the electron transport region. Carriers, such as the holes and the electrons, recombine in the emission layer to produce excitons. These excitons transition and decay from an excited state to a ground state, thereby generating light.

### SUMMARY

One or more aspects of embodiments of the present disclosure are directed toward a light-emitting device including an amine-containing compound, an electronic apparatus including the light-emitting device, and the amine-containing compound.

Additional aspects will be set forth in part in the description which follows and, in part, will be apparent from the description, or may be learned by practice of the presented embodiments of the present disclosure.

According to one or more embodiments of the present disclosure, a light-emitting device includes a first electrode, a second electrode facing the first electrode, an interlayer between the first electrode and the second electrode and including an emission layer, and an amine-containing compound represented by Formula 1: Formula 1

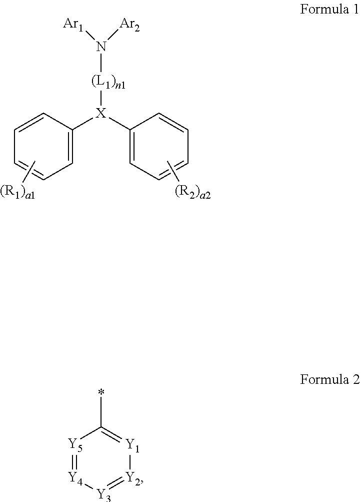

wherein, in Formulae 1 and 2,

X may be B or N,

L₁ may be a C₃-C₆₀ carbocyclic group or a C₁-C₆₀ heterocyclic group, each unsubstituted or substituted with at least one R₁₀ₐ,

n1 may be an integer from 1 to 5,

a1 and a2 may each independently be an integer from 1 to 5,

Ar₁ may be a group represented by Formula 2,

Ar₂ may be a C₆-C₆₀ aryl group or a C₁-C₆₀ heteroaryl group, each unsubstituted or substituted with at least one R₁₀ₐ,

Y₁ may be C(R₁₁) or N, Y₂ may be C(R₁₂) or N, Y₃ may be C(R₁₃) or N, Y₄ may be C(R₁₄) or N, Y₅ may be C(R₁₅) or N,

at least two selected from among Y₁ to Y₅ may each be N,

* indicates a binding site to a neighboring nitrogen atom,

R₁, R₂, and R₁₁ to R₁₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂—Co alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments of the present disclosure, an electronic apparatus and electronic equipment each include the light-emitting device.

According to one or more embodiments of the present disclosure, an amine-containing compound represented by Formula 1 is provided.

### BRIEF DESCRIPTION OF DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a structure of a light-emitting device according to one or more embodiments of the present disclosure;

FIG. 2 is a schematic view of a structure of a light emitting apparatus according to one or more embodiments of the present disclosure;

FIG. 3 is a schematic view of a structure of a light emitting apparatus according to one or more embodiments of the present disclosure;

FIG. 4 is a schematic view of an electronic apparatus according to one or more embodiments of the present disclosure;

FIG. 5 is a diagram schematically illustrating an exterior of a vehicle as electronic equipment including a light-emitting device according to one or more embodiments of the present disclosure; and

FIGS. 6A, 6B, and 6C are each a diagram schematically illustrating an interior of a vehicle according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

According to one or more embodiments of the present disclosure, a light-emitting device may include: a first electrode; a second electrode facing the first electrode; an interlayer between the first electrode and the second electrode and including an emission layer; and a amine-containing compound represented by Formula 1:

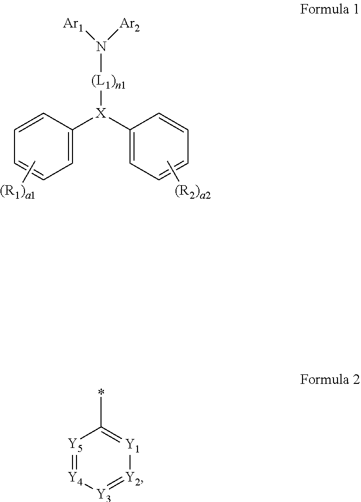

wherein, in Formulae 1 and 2,

X may be B or N,

L₁ may be a C₃-C₆₀ carbocyclic group or a C₁-C₆₀ heterocyclic group, each unsubstituted or substituted with at least one R₁₀ₐ,

n1 may be an integer from 1 to 5,

a1 and a2 may each independently be an integer from 1 to 5,

Ar₁ may be a group represented by Formula 2,

Ar₂ may be a C₆-C₆₀ aryl group or a C₁-C₆₀ heteroaryl group, each unsubstituted or substituted with at least one R₁₀ₐ,

Y₁ may be C(R₁₁) or N, Y₂ may be C(R₁₂) or N, Y₃ may be C(R₁₃) or N, Y₄ may be C(R₁₄) or N, Y₅ may be C(R₁₅) or N,

at least two selected from among Y₁ to Y₅ may each be N,

* indicates a binding site to a neighboring nitrogen atom,

R₁, R₂, and R₁₁ to R₁₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂—Co alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —CI; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, Ar₁ and Ar₂ may be identical to each other.

In one or more embodiments, Ar₁ and Ar₂ may be different from each other.

In one or more embodiments, the amine-containing compound may include at least one deuterium (D).

In one or more embodiments, Formula 2 may be represented by one selected from among Formulae 2-1 to 2-4:

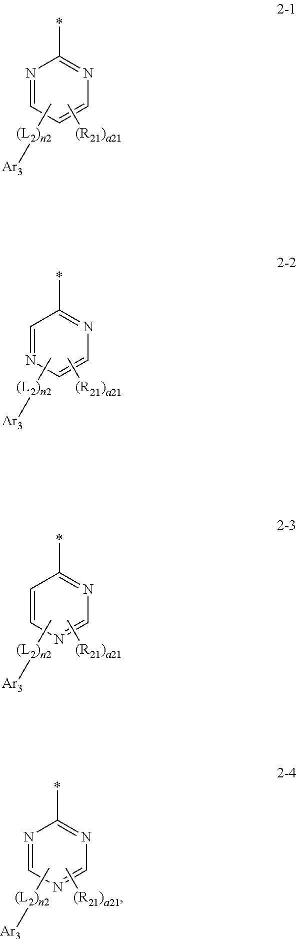

wherein, in Formulae 2-1 to 2-4,

L₂ may be the same as defined herein with respect to L₁,

R₂₁ may be the same as defined herein with respect to R₁,

Ar₃ may be the same as defined herein with respect to Ar₂,

a21 may be an integer from 1 to 3, and

n2 may be an integer from 0 to 4.

In one or more embodiments, L₁ may be a cyclopentadiene group, an adamantane group, a norbornane group, a benzene group, a pentalene group, a naphthalene group, an azulene group, an indacene group, an acenaphthylene group, a phenylene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group, each unsubstituted or substituted with at least one R₁₀ₐ.

In one or more embodiments, L₁ may be a group represented by any one selected from among Formulae 3-1 to 3-6:

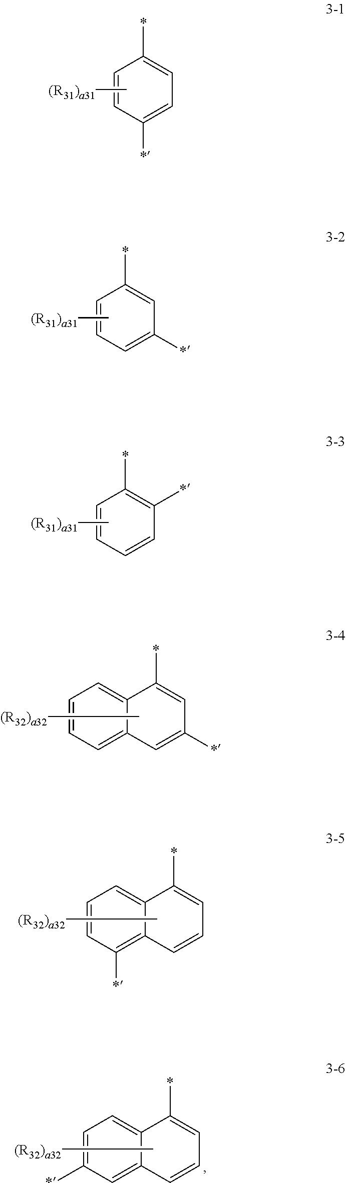

wherein, in Formulae 3-1 to 3-6,

R₃₁ and R₃₂ may each independently be the same as defined herein with respect to R₁,

a31 may be an integer from 1 to 4, a32 may be an integer from 1 to 6,

at least one of R₁ in the number of a1 may optionally be linked to at least one of R₃₁ in the number of a31 or at least one of R₃₂ in the number of a32 to form a C₅-C₃₀ carbocyclic group unsubstituted or substituted with at least one R₁₀ₐ or a C₁-C₃₀ heterocyclic group unsubstituted or substituted with at least one R₁₀ₐ,

at least one of R₂ in the number of a2 may optionally be linked to at least one of R₃₁ in the number of a31 or at least one of R₃₂ in the number of a32 to form a C₅-C₃₀ carbocyclic group unsubstituted or substituted with at least one R₁₀ₐ or a C₁-C₃₀ heterocyclic group unsubstituted or substituted with at least one R₁₀ₐ, and

* and *′ each indicate a binding site to a neighboring atom.

In one or more embodiments, R₃₁ and R₃₂ may each independently be:

hydrogen; deuterium; or

a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof.

In one or more embodiments, the amine-containing compound may satisfy any one selected from among Conditions i to iv:

### Condition i)

Y₁ and Y₅ are each N, Y₂ is C(R₁₂), Y₃ is C(R₁₃), and Y₄ is C(R₁₄);

### Condition ii)

Y₁ and Y₃ are each N, Y₂ is C(R₁₂), Y₄ is C(R₁₄), and Y₅ is C(R₁₅);
Condition iii)
Y₁ and Y₄ are each N, Y₂ is C(R₁₂), Y₃ is C(R₁₃), and Y₅ is C(R₁₅); and

### Condition iv)

Y₁, Y₃, and Y₅ are each N, Y₂ is C(R₁₂), and Y₄ is C(R₁₄).

In one or more embodiments, Ar₂ may be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, or a naphthyridinyl group, each unsubstituted or substituted with at least one R₁₀ₐ.

In one or more embodiments, Ar₂ may be a group represented by Formula 2.

In one or more embodiments, R₁ and R₂ may each independently be:

deuterium; or

a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof.

In one or more embodiments, R₁₁ to R₁₅ may each independently be hydrogen, deuterium, —F, a cyano group, a C₁-C₂₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, R₁₁ to R₁₅ may each independently be: hydrogen, deuterium, —F, or a cyano group;

a C₁-C₂₀ alkyl group unsubstituted or substituted with hydrogen, deuterium, —F, a cyano group, or any combination thereof; or

a benzene group, a naphthalene group, a pyridine group, a dibenzofuran group, a dibenzothiophene group, a carbazole group, an azadibenzofuran group, an azadibenzothiophene group, or an azacarbazole group, each unsubstituted or substituted with hydrogen, deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a dibenzofuranyl group, a dibenzothiophenyl group, a carbazolyl group, a fluorenyl group, a dibenzosilolyl group, or any combination thereof.

In one or more embodiments, R₁₁ to R₁₅ may each independently be:

hydrogen;

a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof; or

a benzene group, a naphthalene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, a 1,2-dimethylpropyl group, a phenyl group, or any combination thereof, each being unsubstituted or substituted with deuterium, —F, —Cl, —Br, —I, a hydroxyl group, a cyano group, a nitro group, or any combination thereof.

In one or more embodiments, R₁₁ to R₁₅ may each independently be: hydrogen, deuterium, —F, —Cl, —Br, —I, a cyano group, or a C₁-C₂₀ alkyl group;

a C₁-C₂₀ alkyl group substituted with at least one of deuterium, —F, —Cl, —Br, —I, —CD₃, —CD₂H, —CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, or a pyrimidinyl group;

a phenyl group, a pyridine group, a dibenzofuranyl group, a dibenzothiophenyl group, a carbazolyl group, an azadibenzofuranyl group, an azadibenzothiophenyl group, or an azacarbazolyl group, each unsubstituted or substituted with at least one of deuterium, —F, —Cl, —Br, —I, —CD₃, —CD₂H, —CDH₂, —CF₃, —CF₂H, —CFH₂, a cyano group, a C₁-C₂₀ alkyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), or —B(Q₃₁)(Q₃₂); or

—Si(Q₁)(Q₂)(Q₃), —N(Q₁)(Q₂), or —B(Q₁)(Q₂), and

Q₁ to Q₃ and Q₃₁ to Q₃₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, or a triazinyl group.

In one or more embodiments, R₁₁ to R₁₅ may each independently be:

a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof; or

a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, a 1,2-dimethylpropyl group, a phenyl group, or any combination thereof, each unsubstituted or substituted with deuterium, —F, —Cl, —Br, —I, a hydroxyl group, a cyano group, a nitro group, or any combination thereof.

In one or more embodiments, the amine-containing compound represented by Formula 1 may be any one selected from among Compounds 1 to 694:

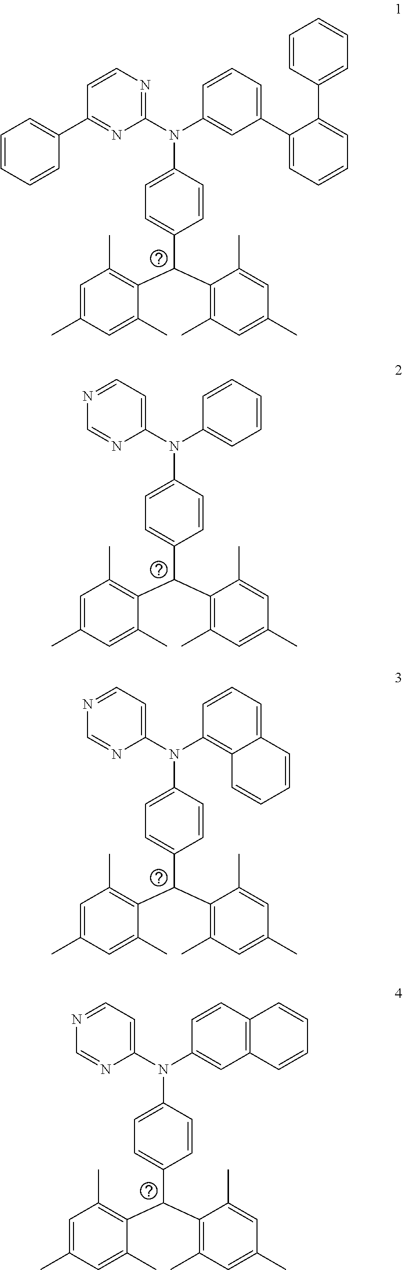

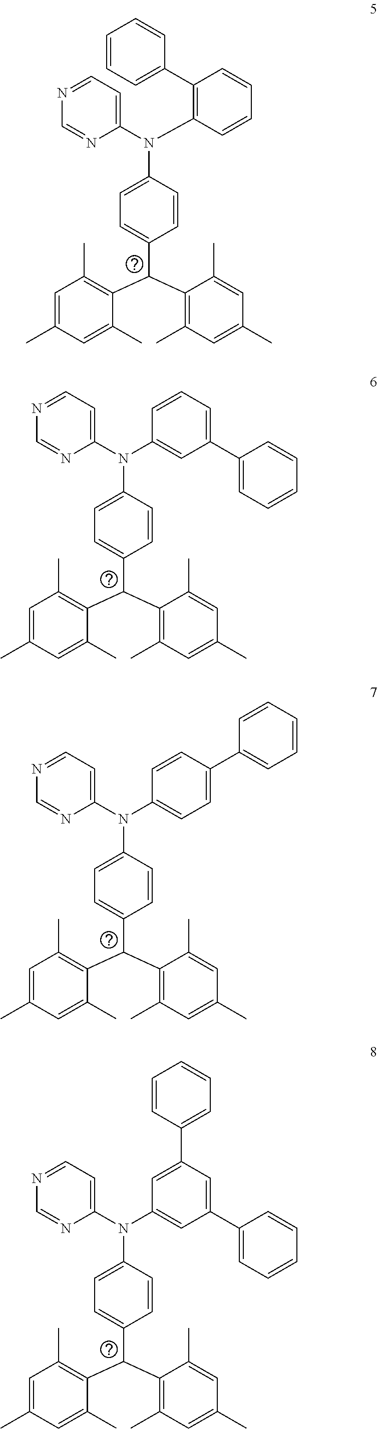

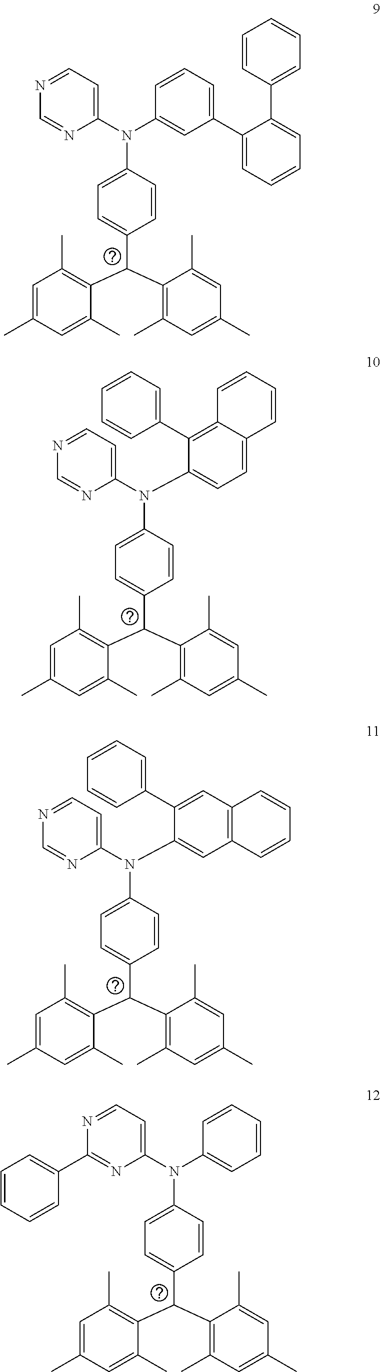

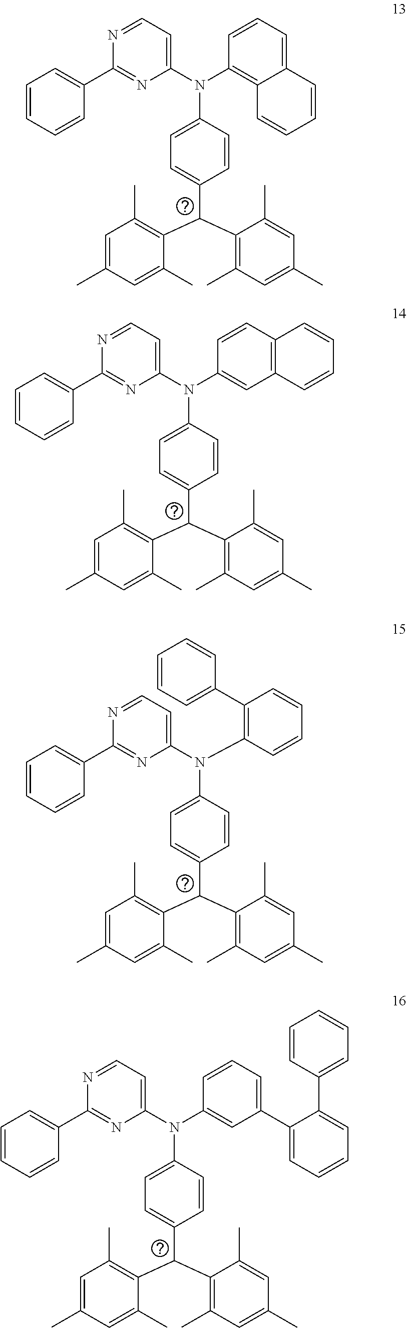

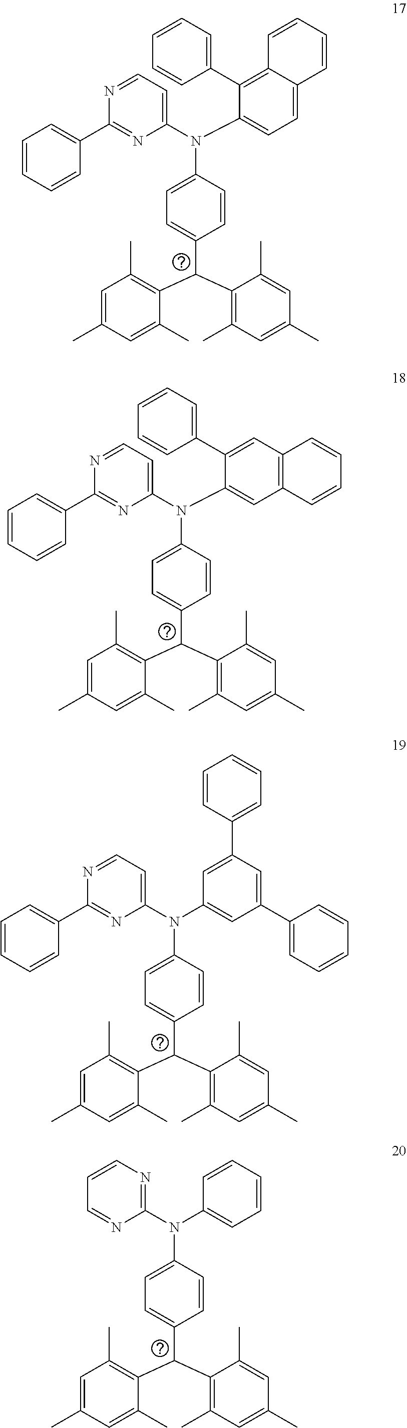

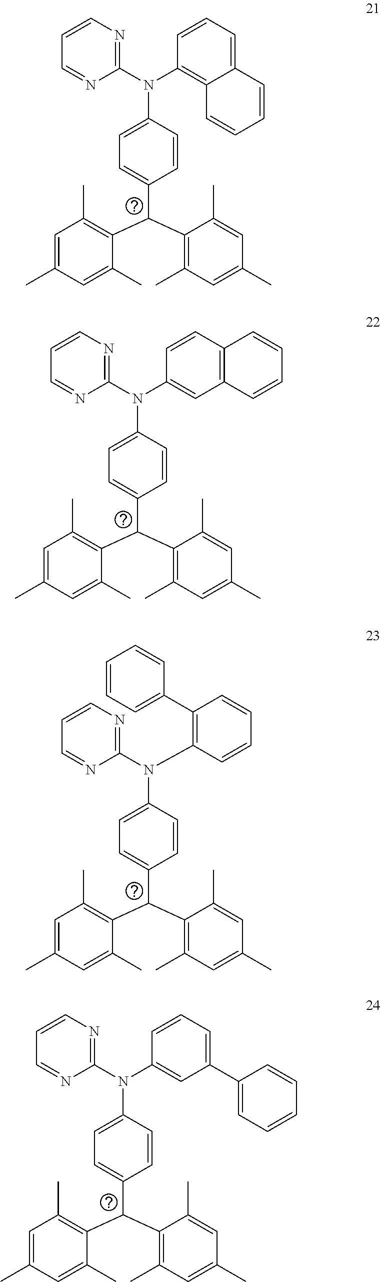

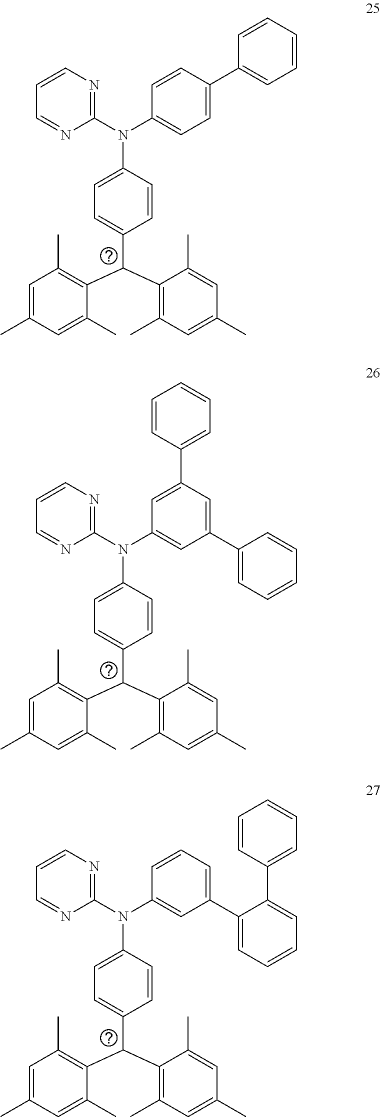 [SMILES: Cc1cc(C)c(C(c2ccc(N(c3ccc(-c4ccccc4)cc3)c3ncccn3)cc2)c2c(C)cc(C)cc2C)c(C)c1 ; Cc1cc(C)c(C(c2ccc(N(c3cc(-c4ccccc4)cc(-c4ccccc4)c3)c3ncccn3)cc2)c2c(C)cc(C)cc2C)c(C)c1 ; Cc1cc(C)c(C(c2ccc(N(c3cccc(-c4ccccc4-c4ccccc4)c3)c3ncccn3)cc2)c2c(C)cc(C)cc2C)c(C)c1]

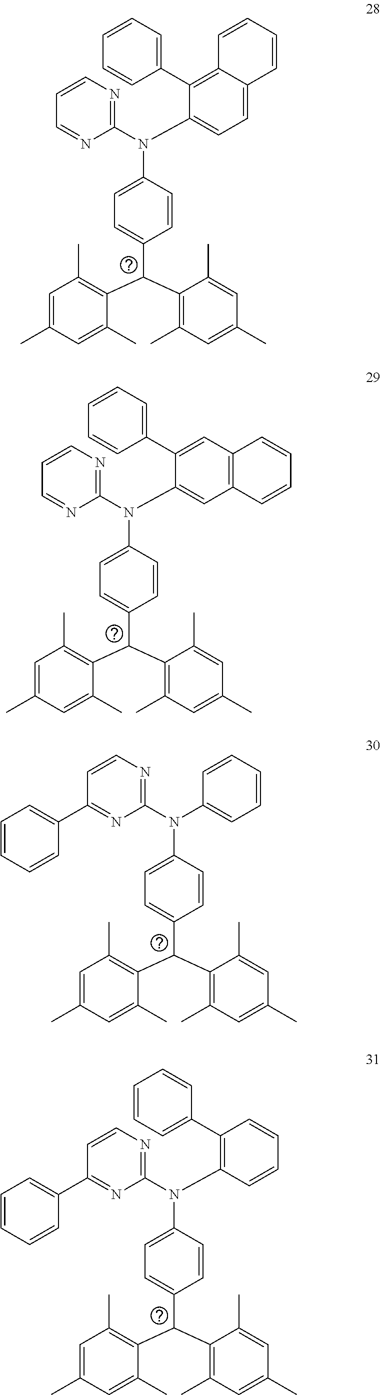

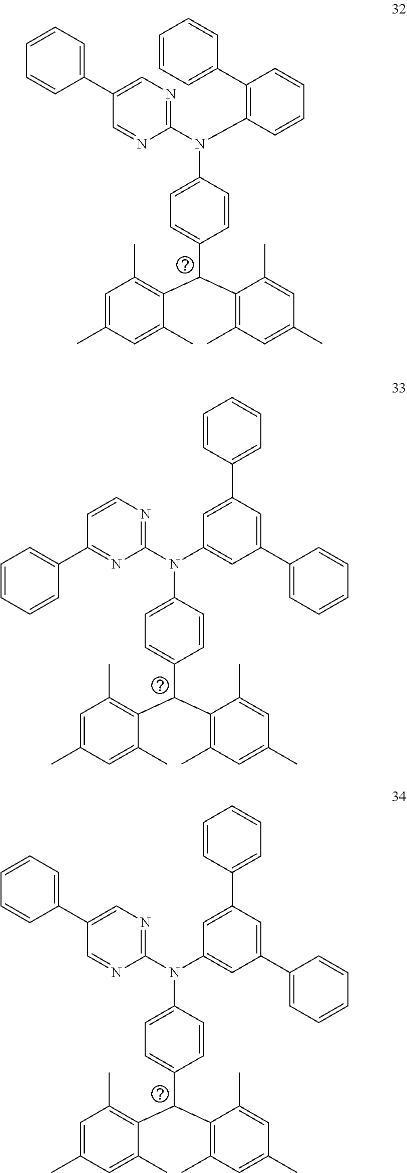

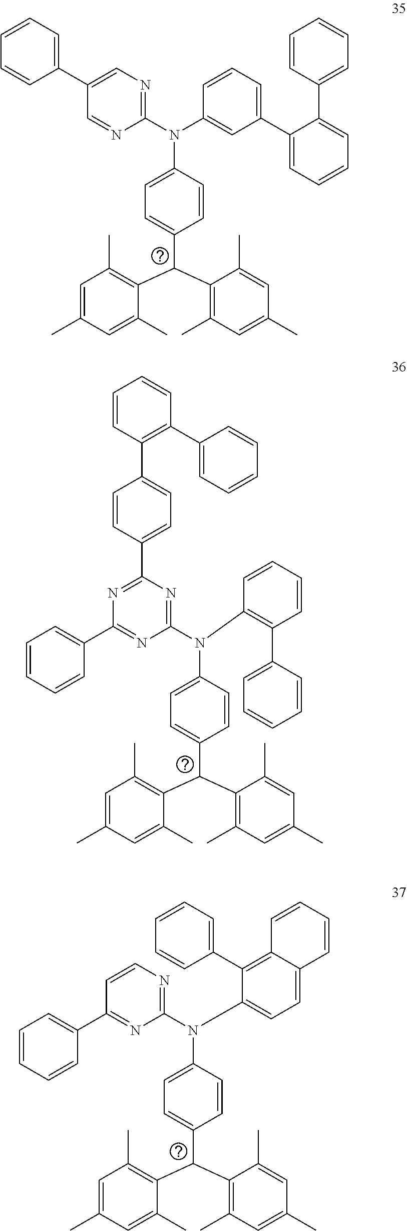 [SMILES: Cc1cc(C)c(C(c2ccc(N(c3cccc(-c4ccccc4-c4ccccc4)c3)c3ncc(-c4ccccc4)cn3)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3ccccc3-c3ccccc3)cc2)c2c(C)cc(C)cc2C)c(C)c1 ; Cc1cc(C)c(C(c2ccc(N(c3nccc(-c4ccccc4)n3)c3ccc4ccccc4c3-c3ccccc3)cc2)c2c(C)cc(C)cc2C)c(C)c1]

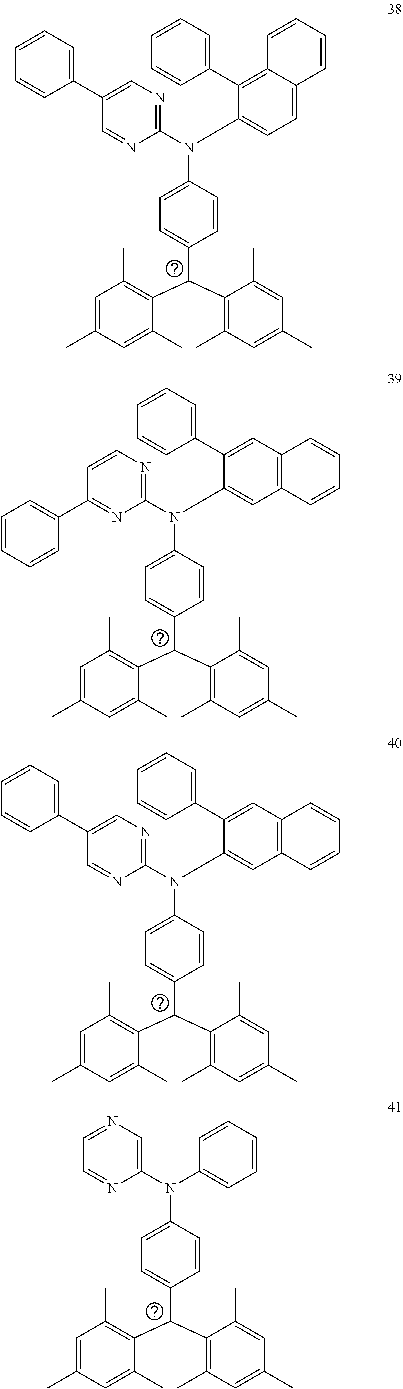

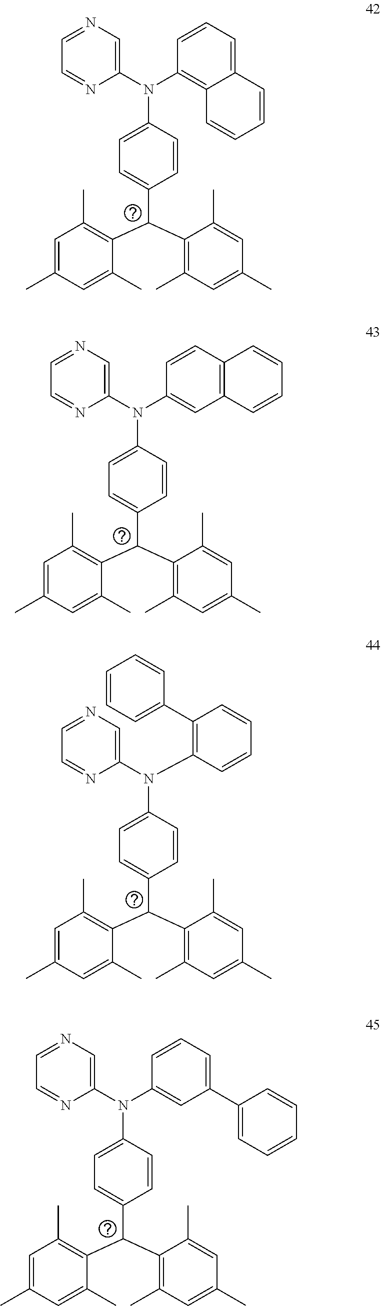

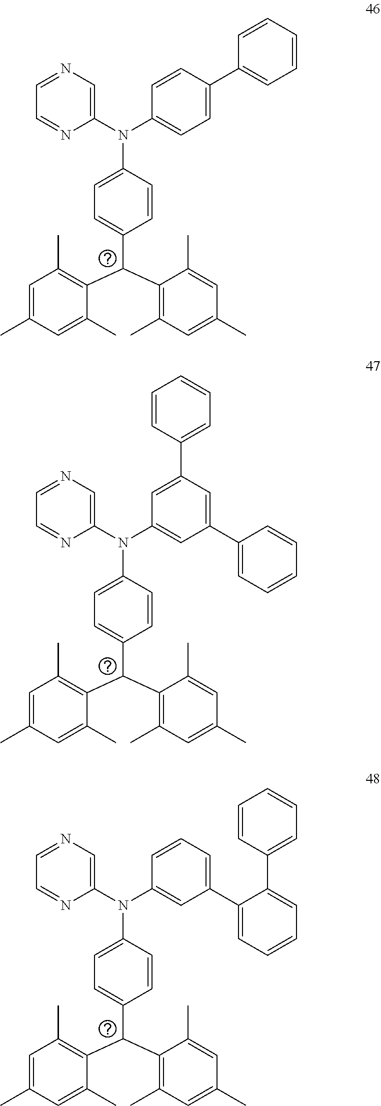

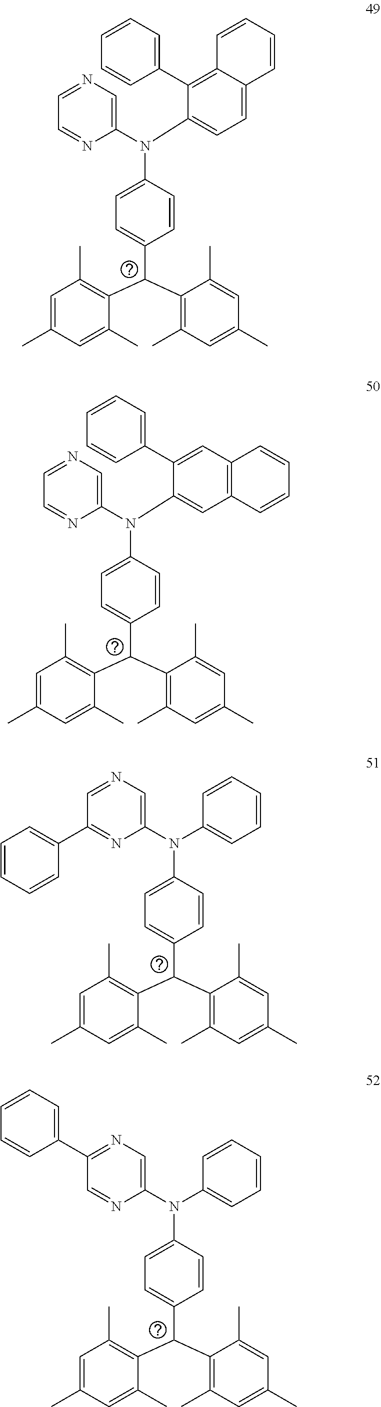 [SMILES: Cc1cc(C)c(C(c2ccc(N(c3cnccn3)c3ccc4ccccc4c3-c3ccccc3)cc2)c2c(C)cc(C)cc2C)c(C)c1 ; Cc1cc(C)c(C(c2ccc(N(c3cnccn3)c3cc4ccccc4cc3-c3ccccc3)cc2)c2c(C)cc(C)cc2C)c(C)c1 ; Cc1cc(C)c(C(c2ccc(N(c3ccccc3)c3cncc(-c4ccccc4)n3)cc2)c2c(C)cc(C)cc2C)c(C)c1 ; Cc1cc(C)c(C(c2ccc(N(c3ccccc3)c3cnc(-c4ccccc4)cn3)cc2)c2c(C)cc(C)cc2C)c(C)c1]

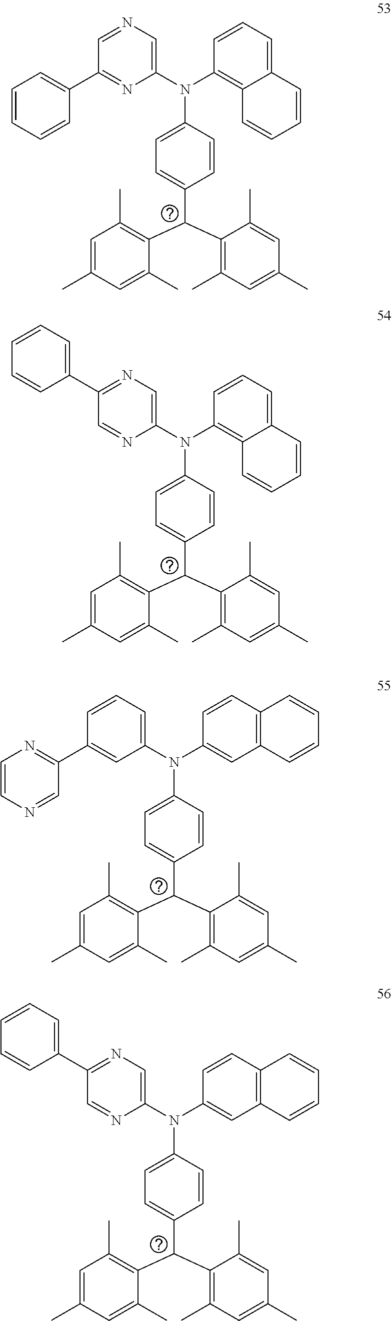

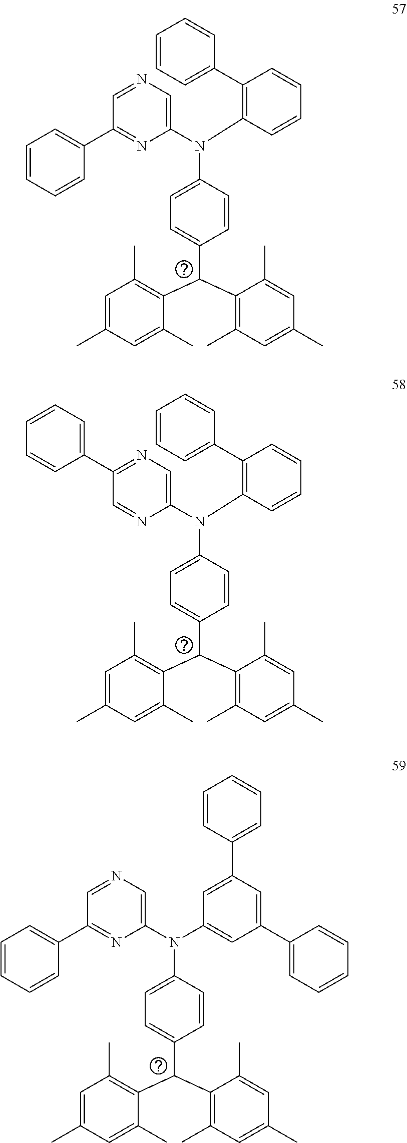 [SMILES: Cc1cc(C)c(C(c2ccc(N(c3cncc(-c4ccccc4)n3)c3ccccc3-c3ccccc3)cc2)c2c(C)cc(C)cc2C)c(C)c1 ; Cc1cc(C)c(C(c2ccc(N(c3cnc(-c4ccccc4)cn3)c3ccccc3-c3ccccc3)cc2)c2c(C)cc(C)cc2C)c(C)c1 ; Cc1cc(C)c(C(c2ccc(N(c3cc(-c4ccccc4)cc(-c4ccccc4)c3)c3cncc(-c4ccccc4)n3)cc2)c2c(C)cc(C)cc2C)c(C)c1]

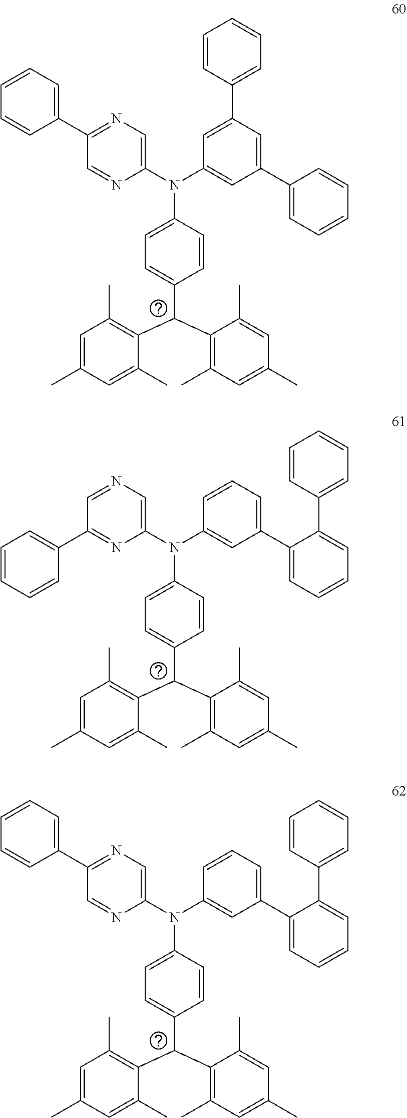 [SMILES: Cc1cc(C)c(C(c2ccc(N(c3cc(-c4ccccc4)cc(-c4ccccc4)c3)c3cnc(-c4ccccc4)cn3)cc2)c2c(C)cc(C)cc2C)c(C)c1 ; Cc1cc(C)c(C(c2ccc(N(c3cccc(-c4ccccc4-c4ccccc4)c3)c3cncc(-c4ccccc4)n3)cc2)c2c(C)cc(C)cc2C)c(C)c1 ; Cc1cc(C)c(C(c2ccc(N(c3cccc(-c4ccccc4-c4ccccc4)c3)c3cnc(-c4ccccc4)cn3)cc2)c2c(C)cc(C)cc2C)c(C)c1]

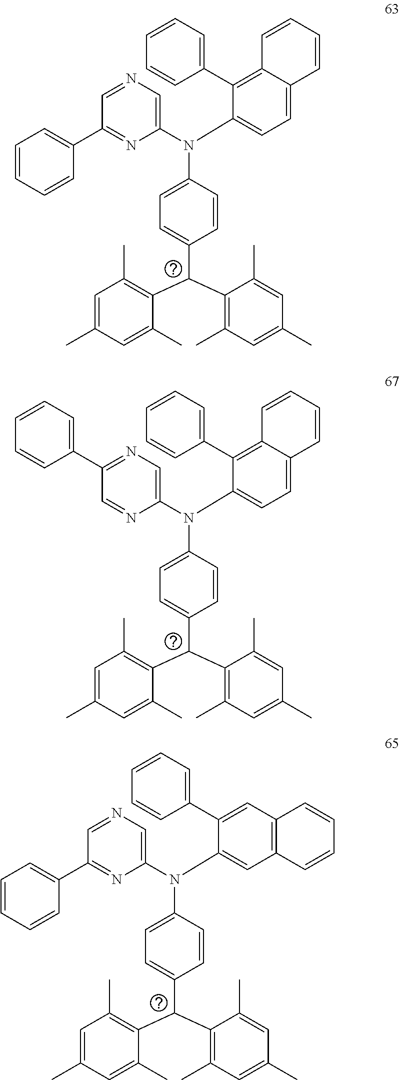

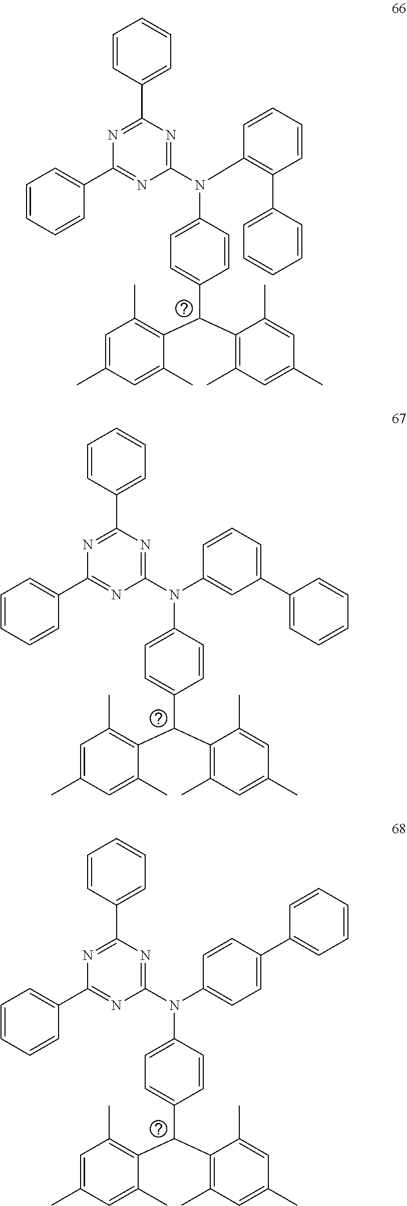

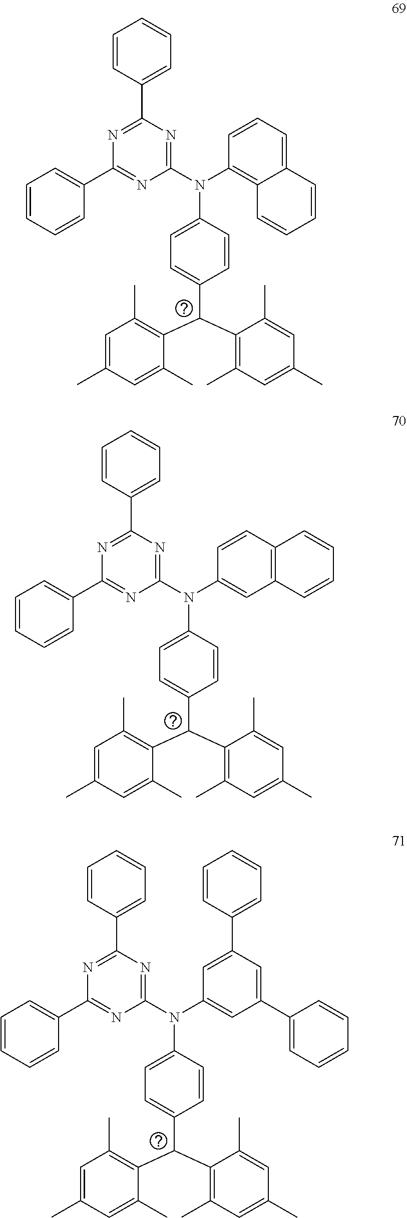

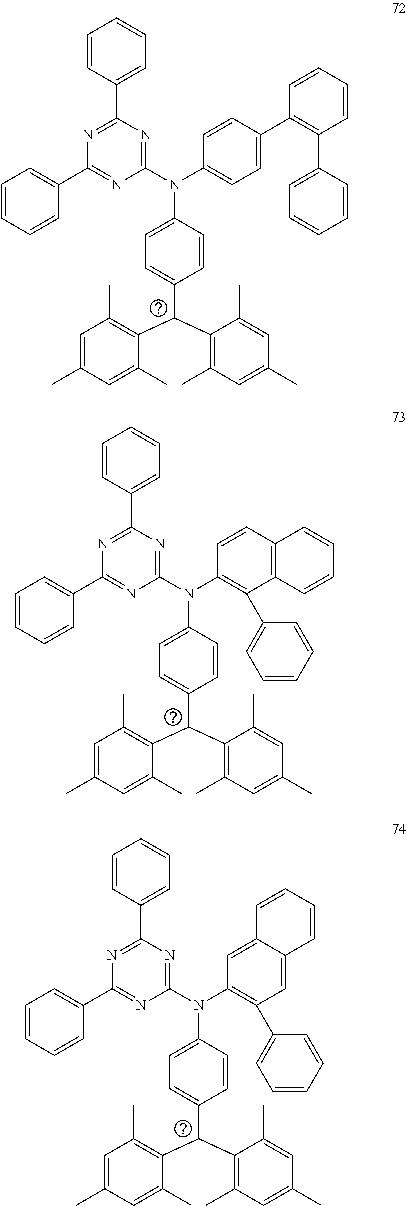 [SMILES: Cc1cc(C)c(C(c2ccc(N(c3ccc(-c4ccccc4-c4ccccc4)cc3)c3nc(-c4ccccc4)nc(-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4ccccc4)n3)c3cc4ccccc4cc3-c3ccccc3)cc2)c2c(C)cc(C)cc2C)c(C)c1]

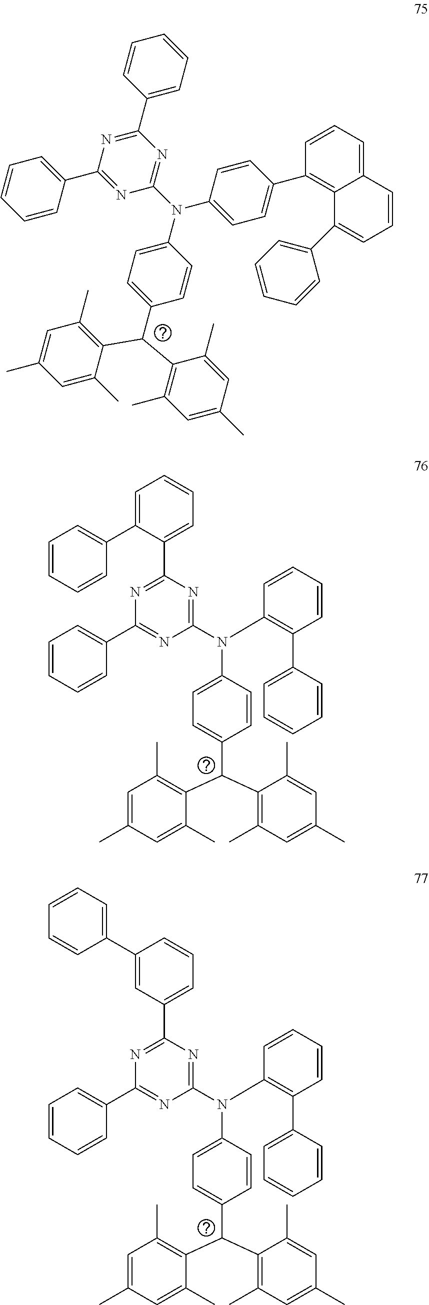 [SMILES: Cc1cc(C)c(C(c2ccc(N(c3ccc(-c4cccc5cccc(-c6ccccc6)c45)cc3)c3nc(-c4ccccc4)nc(-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3ccccc3-c3ccccc3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3ccccc3-c3ccccc3)cc2)c2c(C)cc(C)cc2C)c(C)c1]

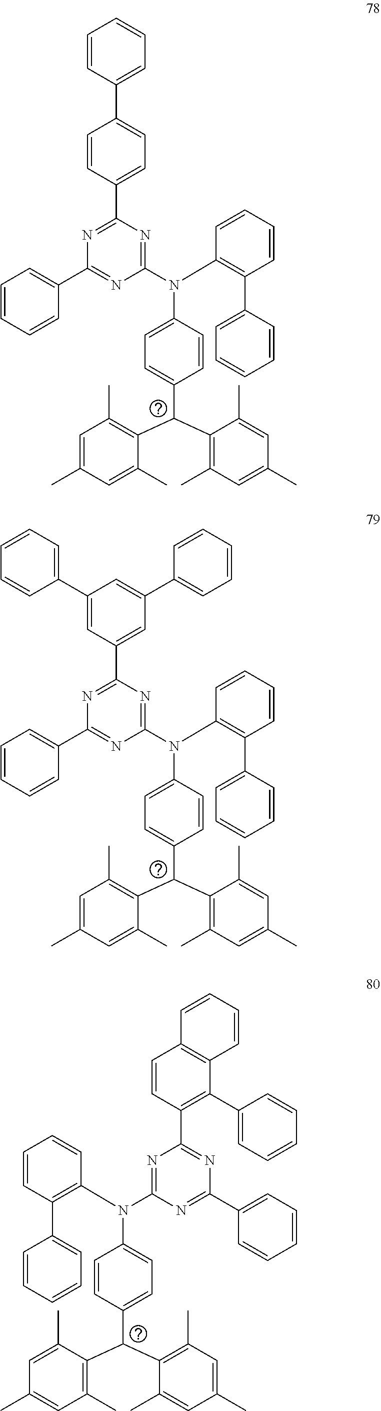

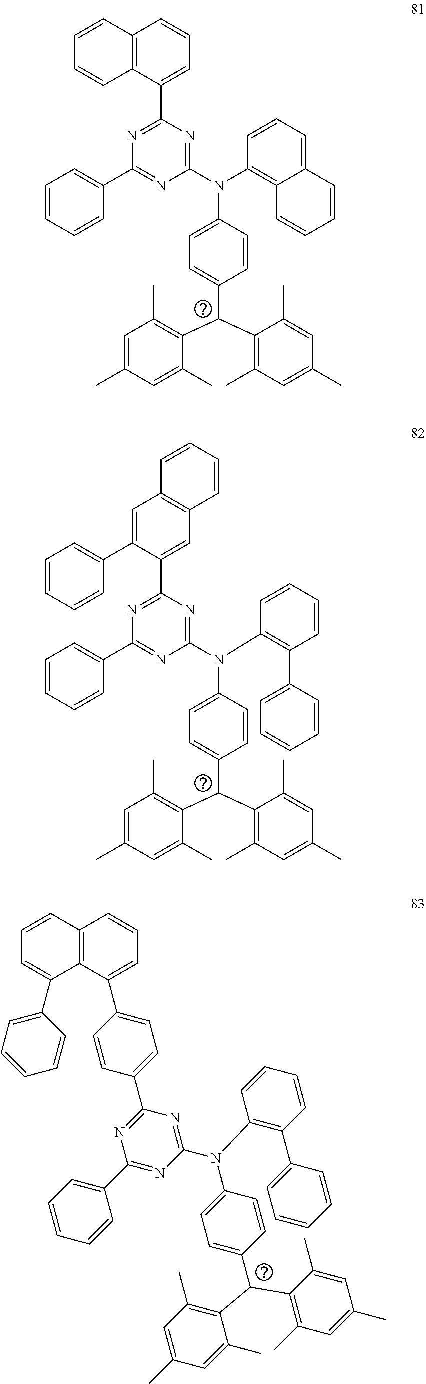 [SMILES: Cc1cc(C)c(C(c2ccc(N(c3nc(-c4ccccc4)nc(-c4cccc5ccccc45)n3)c3cccc4ccccc34)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3ccccc3-c3ccccc3)cc2)c2c(C)cc(C)cc2C)c(C)c1 ; Cc1cc(C)c(C(c2ccc(N(c3nc(-c4ccccc4)nc(-c4ccc(-c5cccc6cccc(-c7ccccc7)c56)cc4)n3)c3ccccc3-c3ccccc3)cc2)c2c(C)cc(C)cc2C)c(C)c1]

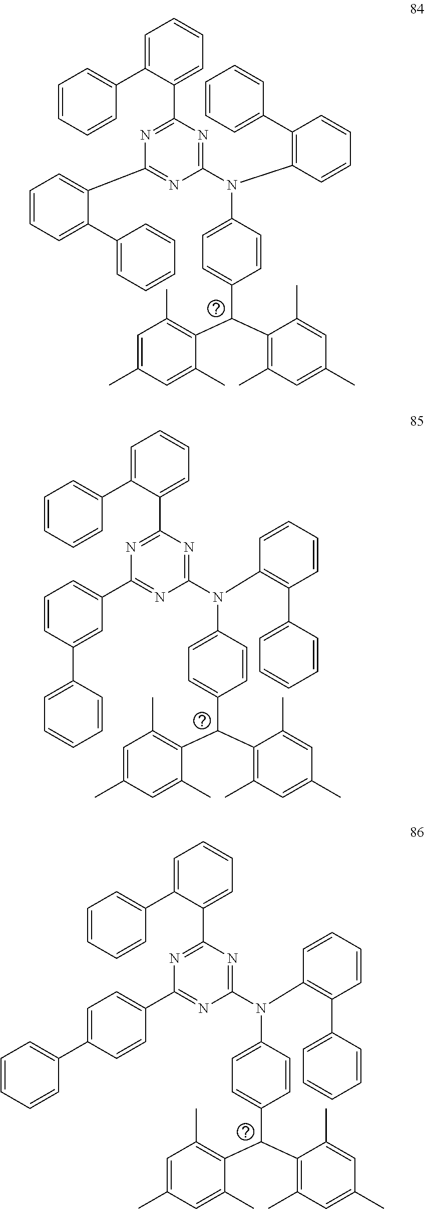

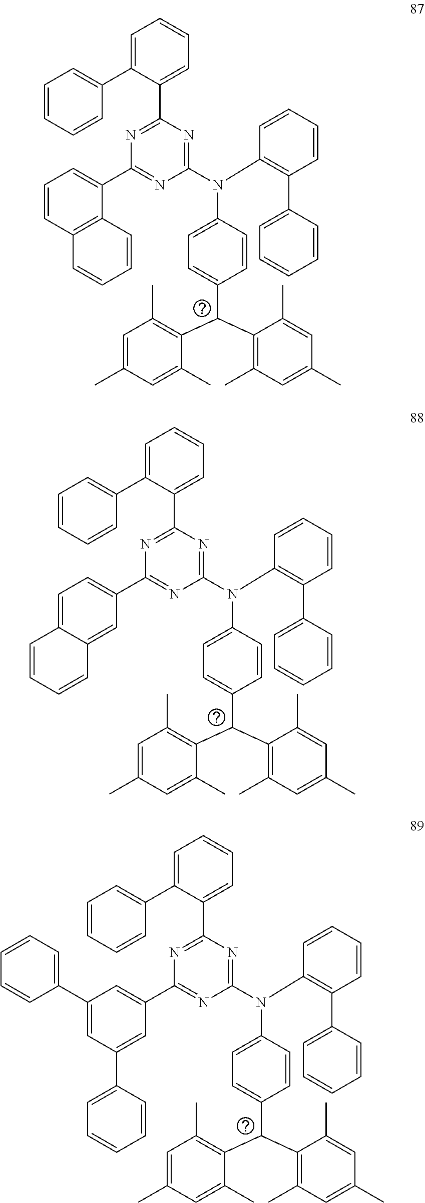 [SMILES: Cc1cc(C)c(C(c2ccc(N(c3nc(-c4ccccc4-c4ccccc4)nc(-c4cccc5ccccc45)n3)c3ccccc3-c3ccccc3)cc2)c2c(C)cc(C)cc2C)c(C)c1 ; Cc1cc(C)c(C(c2ccc(N(c3nc(-c4ccc5ccccc5c4)nc(-c4ccccc4-c4ccccc4)n3)c3ccccc3-c3ccccc3)cc2)c2c(C)cc(C)cc2C)c(C)c1 ; Cc1cc(C)c(C(c2ccc(N(c3nc(-c4cc(-c5ccccc5)cc(-c5ccccc5)c4)nc(-c4ccccc4-c4ccccc4)n3)c3ccccc3-c3ccccc3)cc2)c2c(C)cc(C)cc2C)c(C)c1]

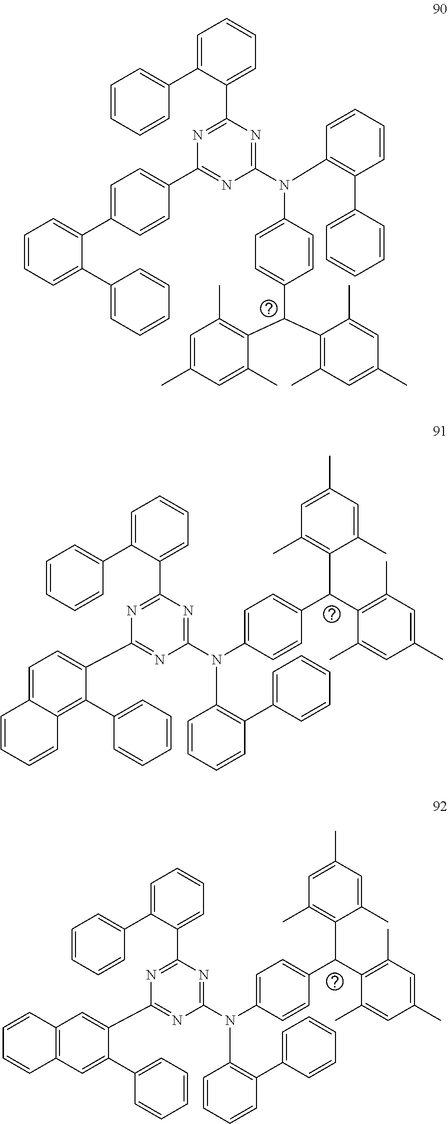 [SMILES: Cc1cc(C)c(C(c2ccc(N(c3nc(-c4ccc(-c5ccccc5-c5ccccc5)cc4)nc(-c4ccccc4-c4ccccc4)n3)c3ccccc3-c3ccccc3)cc2)c2c(C)cc(C)cc2C)c(C)c1 ; Cc1cc(C)c(C(c2ccc(N(c3nc(-c4ccccc4-c4ccccc4)nc(-c4ccc5ccccc5c4-c4ccccc4)n3)c3ccccc3-c3ccccc3)cc2)c2c(C)cc(C)cc2C)c(C)c1 ; Cc1cc(C)c(C(c2ccc(N(c3nc(-c4ccccc4-c4ccccc4)nc(-c4cc5ccccc5cc4-c4ccccc4)n3)c3ccccc3-c3ccccc3)cc2)c2c(C)cc(C)cc2C)c(C)c1]

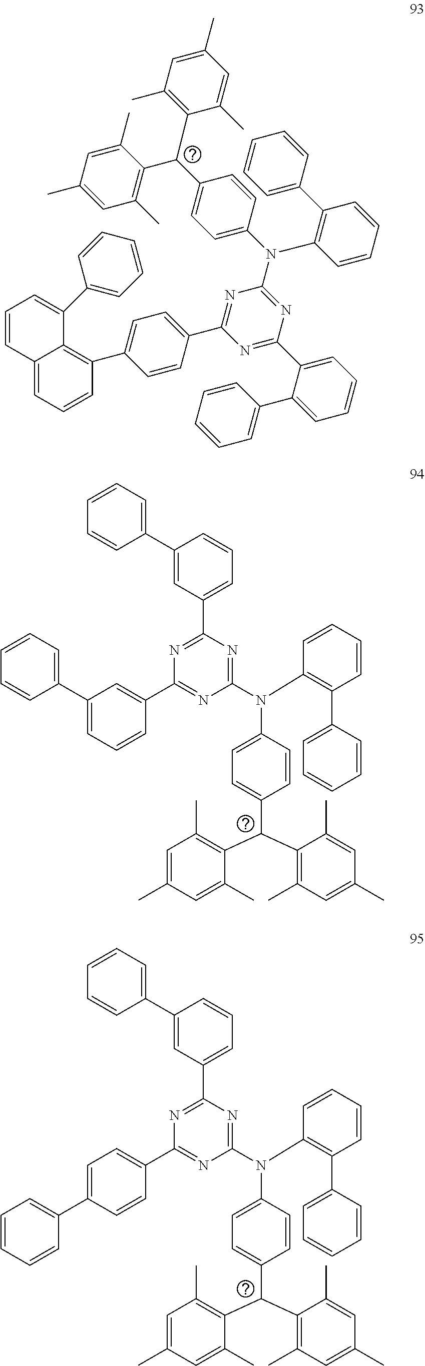 [SMILES: Cc1cc(C)c(C(c2ccc(N(c3nc(-c4ccc(-c5cccc6cccc(-c7ccccc7)c56)cc4)nc(-c4ccccc4-c4ccccc4)n3)c3ccccc3-c3ccccc3)cc2)c2c(C)cc(C)cc2C)c(C)c1 ; Cc1cc(C)c(C(c2ccc(N(c3nc(-c4cccc(-c5ccccc5)c4)nc(-c4cccc(-c5ccccc5)c4)n3)c3ccccc3-c3ccccc3)cc2)c2c(C)cc(C)cc2C)c(C)c1 ; Cc1cc(C)c(C(c2ccc(N(c3nc(-c4ccc(-c5ccccc5)cc4)nc(-c4cccc(-c5ccccc5)c4)n3)c3ccccc3-c3ccccc3)cc2)c2c(C)cc(C)cc2C)c(C)c1]

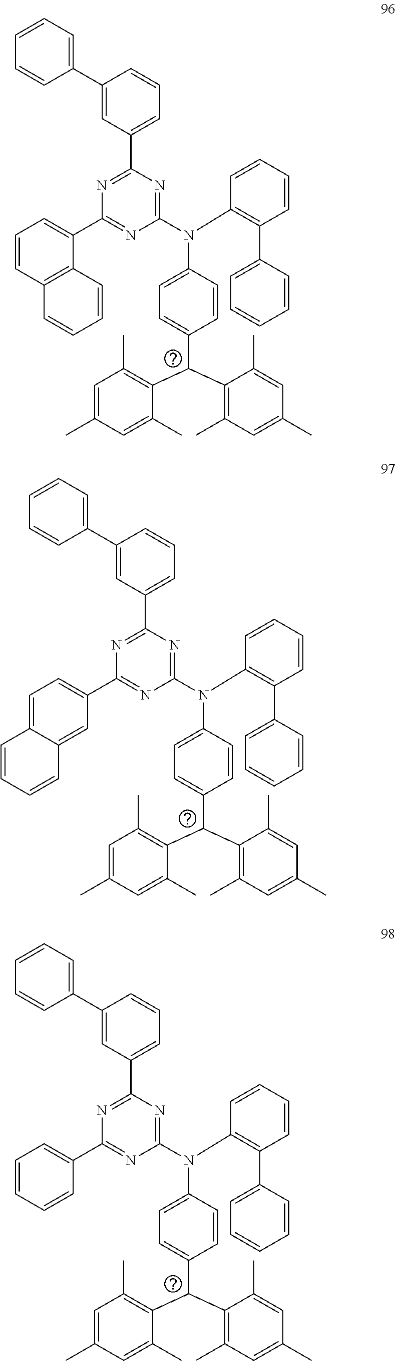

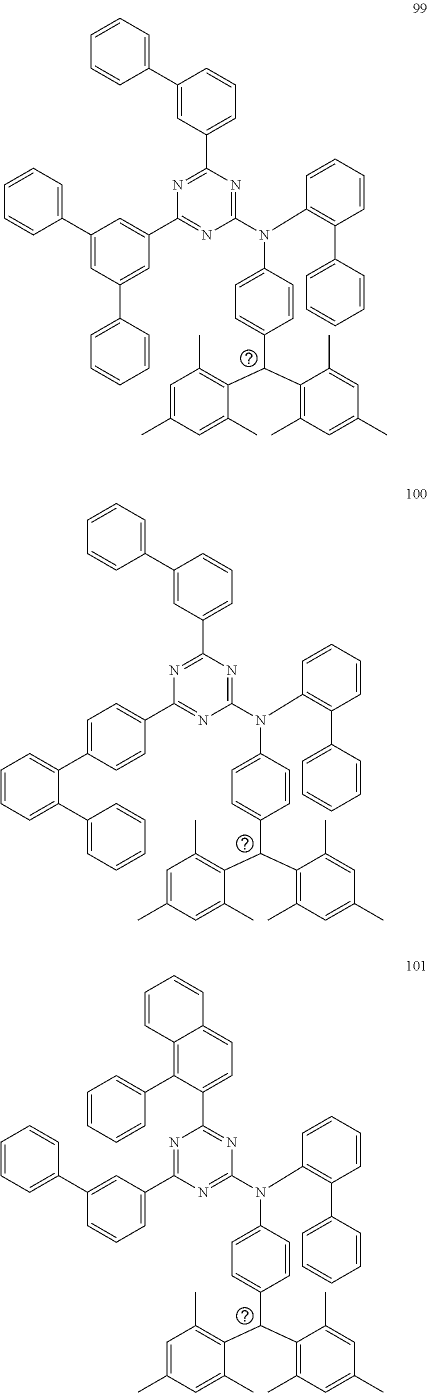 [SMILES: Cc1cc(C)c(C(c2ccc(N(c3nc(-c4cccc(-c5ccccc5)c4)nc(-c4cc(-c5ccccc5)cc(-c5ccccc5)c4)n3)c3ccccc3-c3ccccc3)cc2)c2c(C)cc(C)cc2C)c(C)c1 ; Cc1cc(C)c(C(c2ccc(N(c3nc(-c4ccc(-c5ccccc5-c5ccccc5)cc4)nc(-c4cccc(-c5ccccc5)c4)n3)c3ccccc3-c3ccccc3)cc2)c2c(C)cc(C)cc2C)c(C)c1 ; Cc1cc(C)c(C(c2ccc(N(c3nc(-c4cccc(-c5ccccc5)c4)nc(-c4ccc5ccccc5c4-c4ccccc4)n3)c3ccccc3-c3ccccc3)cc2)c2c(C)cc(C)cc2C)c(C)c1]

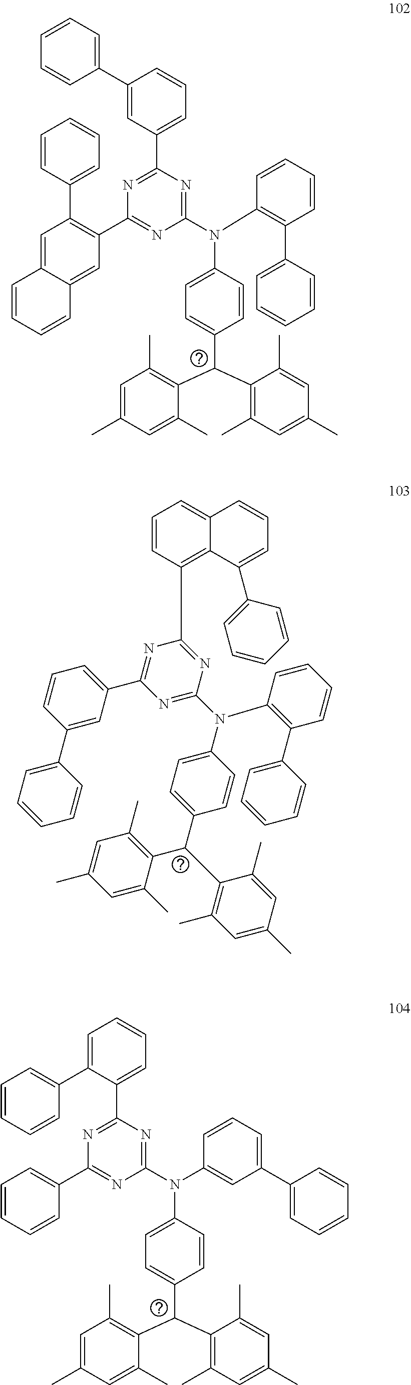 [SMILES: Cc1cc(C)c(C(c2ccc(N(c3nc(-c4cccc(-c5ccccc5)c4)nc(-c4cc5ccccc5cc4-c4ccccc4)n3)c3ccccc3-c3ccccc3)cc2)c2c(C)cc(C)cc2C)c(C)c1 ; Cc1cc(C)c(C(c2ccc(N(c3nc(-c4cccc(-c5ccccc5)c4)nc(-c4cccc5cccc(-c6ccccc6)c45)n3)c3ccccc3-c3ccccc3)cc2)c2c(C)cc(C)cc2C)c(C)c1 ; Cc1cc(C)c(C(c2ccc(N(c3cccc(-c4ccccc4)c3)c3nc(-c4ccccc4)nc(-c4ccccc4-c4ccccc4)n3)cc2)c2c(C)cc(C)cc2C)c(C)c1]

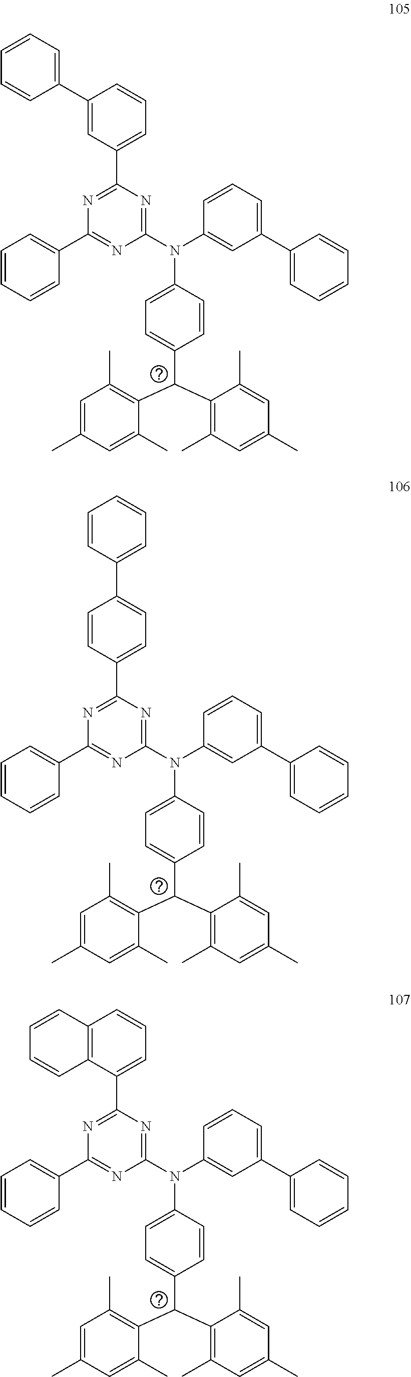

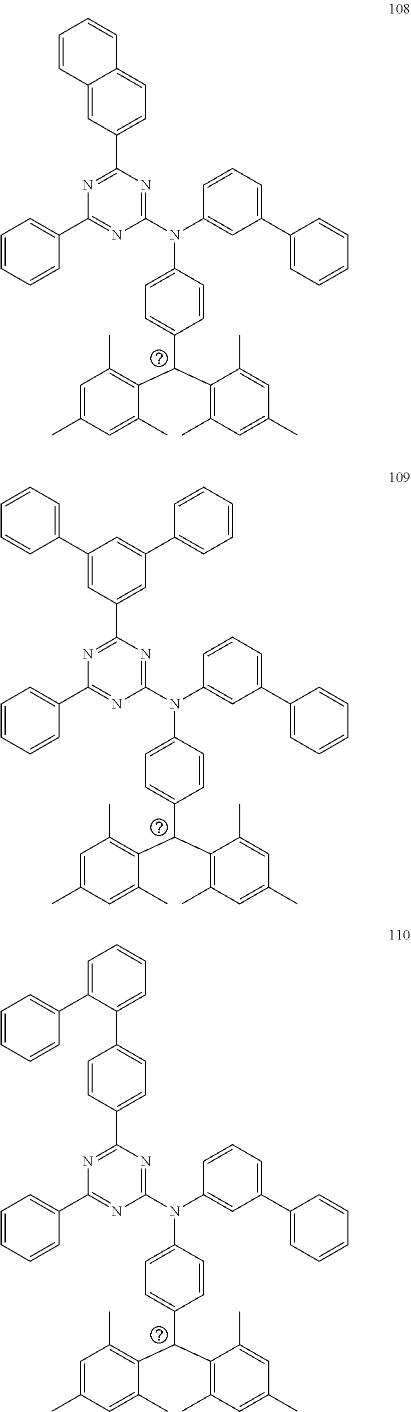

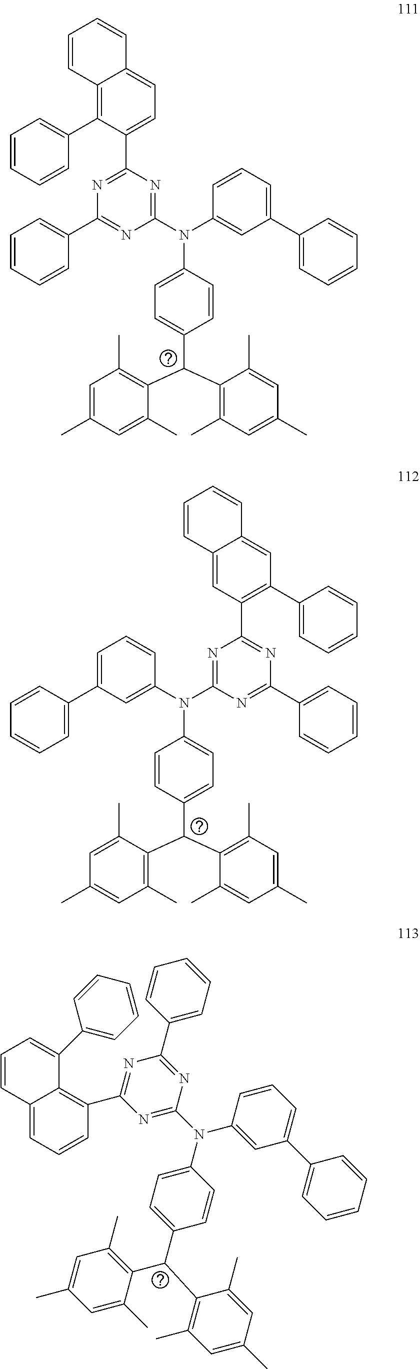 [SMILES: Cc1cc(C)c(C(c2ccc(N(c3cccc(-c4ccccc4)c3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3cccc(-c4ccccc4)c3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3cccc(-c4ccccc4)c3)c3nc(-c4ccccc4)nc(-c4cccc5cccc(-c6ccccc6)c45)n3)cc2)c2c(C)cc(C)cc2C)c(C)c1]

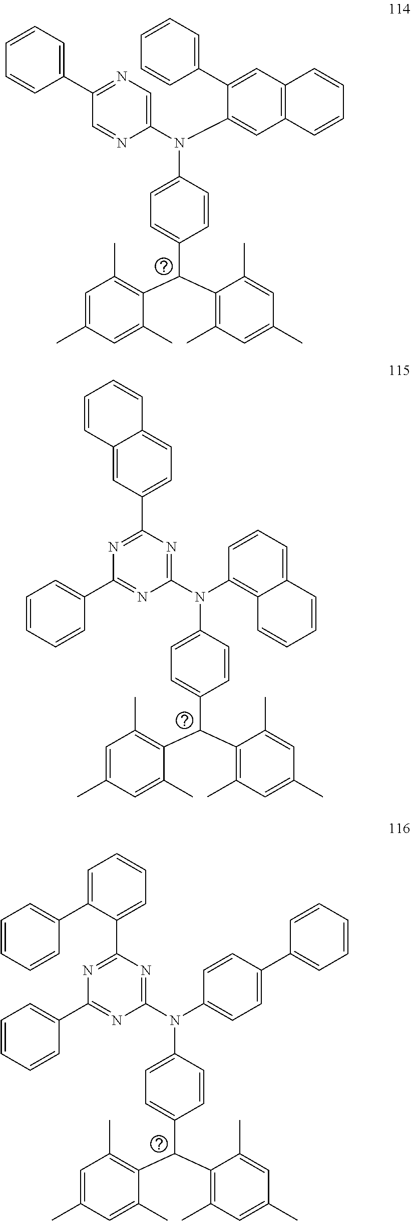

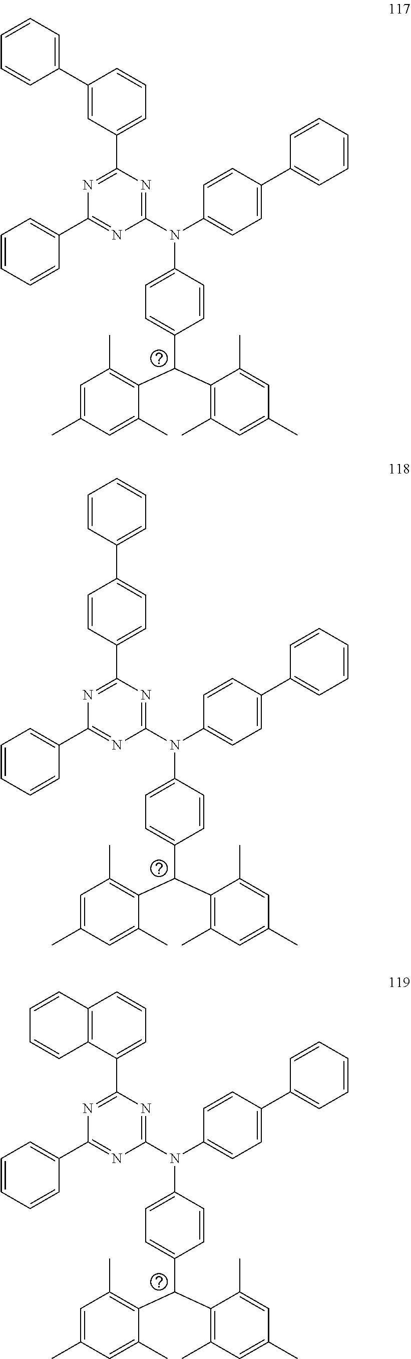

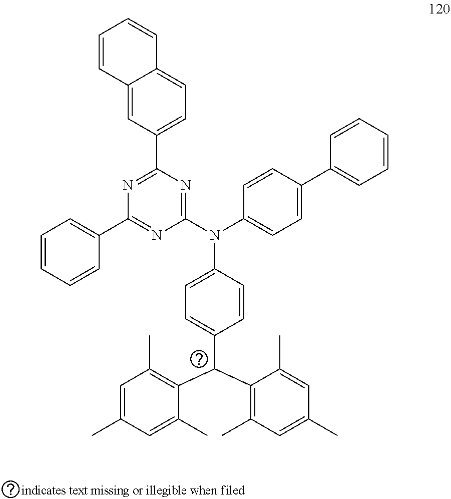 [SMILES: Cc1cc(C)c(C(c2ccc(N(c3ccc(-c4ccccc4)cc3)c3nc(-c4ccccc4)nc(-c4ccc5ccccc5c4)n3)cc2)c2c(C)cc(C)cc2C)c(C)c1]

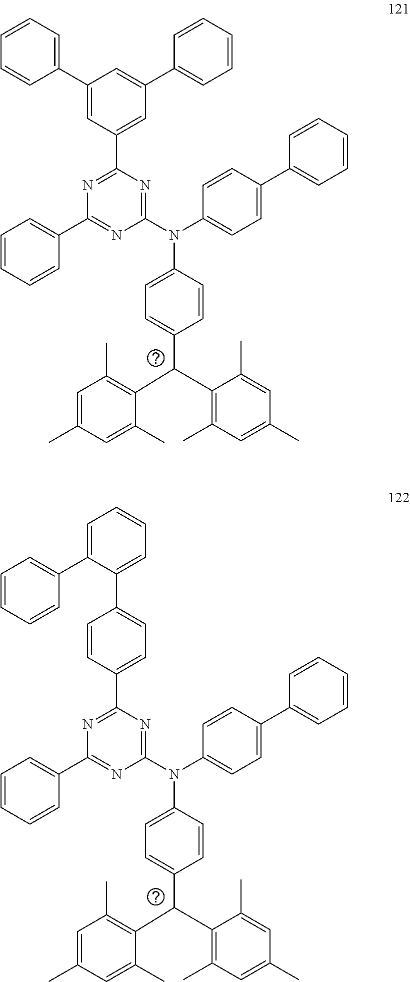 [SMILES: Cc1cc(C)c(C(c2ccc(N(c3ccc(-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc(-c4ccccc4)cc3)c3nc(-c4ccccc4)nc(-c4ccc(-c5ccccc5-c5ccccc5)cc4)n3)cc2)c2c(C)cc(C)cc2C)c(C)c1]

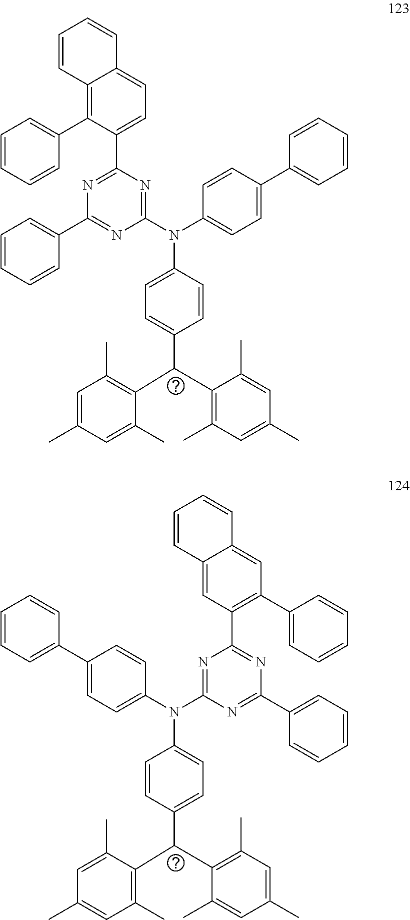 [SMILES: Cc1cc(C)c(C(c2ccc(N(c3ccc(-c4ccccc4)cc3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc(-c4ccccc4)cc3)c3nc(-c4ccccc4)nc(-c4cc5ccccc5cc4-c4ccccc4)n3)cc2)c2c(C)cc(C)cc2C)c(C)c1]

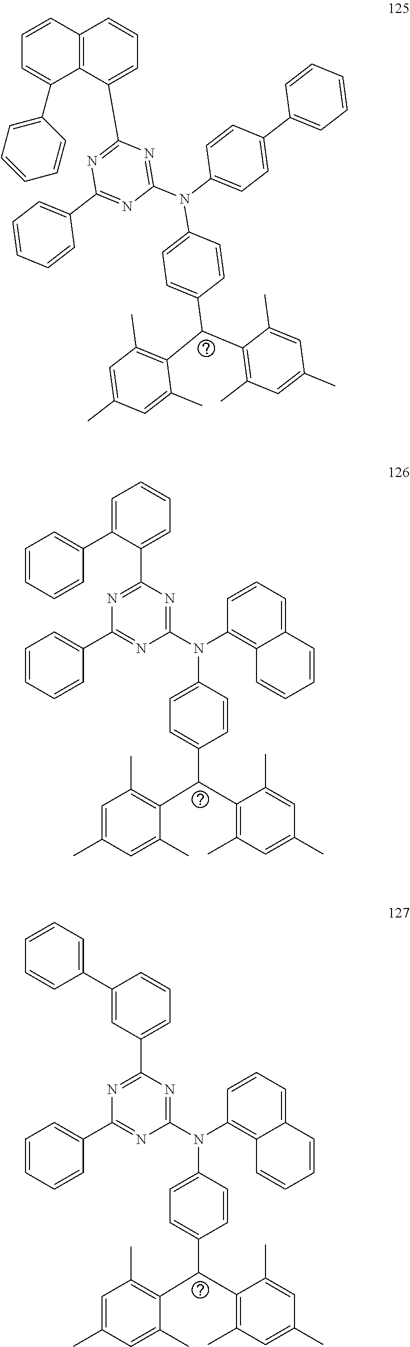 [SMILES: Cc1cc(C)c(C(c2ccc(N(c3ccc(-c4ccccc4)cc3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3cccc4ccccc34)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3cccc4ccccc34)cc2)c2c(C)cc(C)cc2C)c(C)c1]

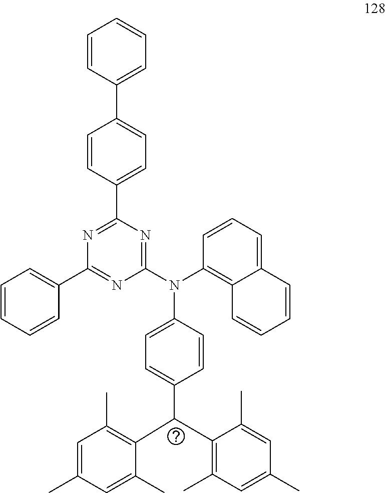 [SMILES: Cc1cc(C)c(C(c2ccc(N(c3nc(-c4ccccc4)nc(-c4ccc(-c5ccccc5)cc4)n3)c3cccc4ccccc34)cc2)c2c(C)cc(C)cc2C)c(C)c1]

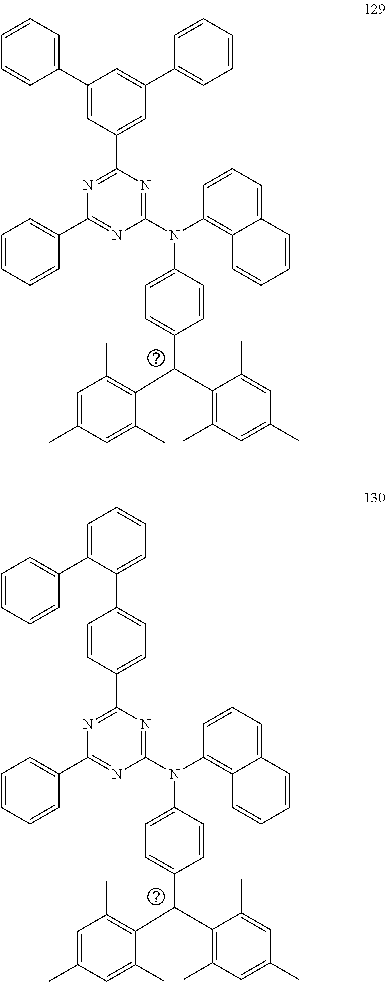 [SMILES: Cc1cc(C)c(C(c2ccc(N(c3nc(-c4ccccc4)nc(-c4cc(-c5ccccc5)cc(-c5ccccc5)c4)n3)c3cccc4ccccc34)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3cccc4ccccc34)cc2)c2c(C)cc(C)cc2C)c(C)c1]

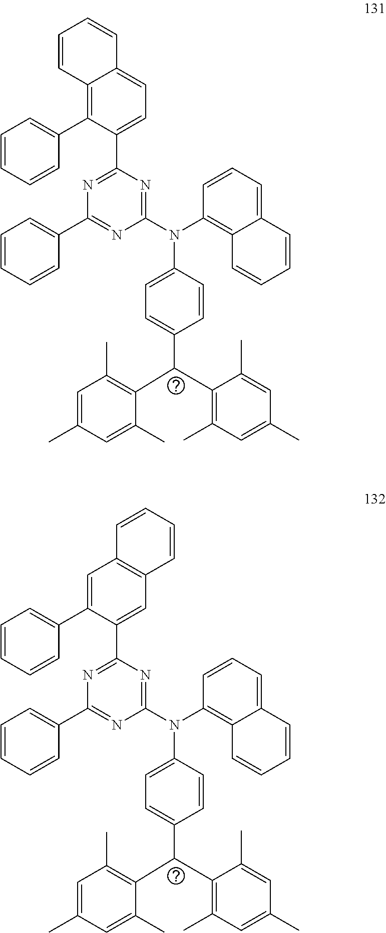 [SMILES: Cc1cc(C)c(C(c2ccc(N(c3nc(-c4ccccc4)nc(-c4ccc5ccccc5c4-c4ccccc4)n3)c3cccc4ccccc34)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3cccc4ccccc34)cc2)c2c(C)cc(C)cc2C)c(C)c1]

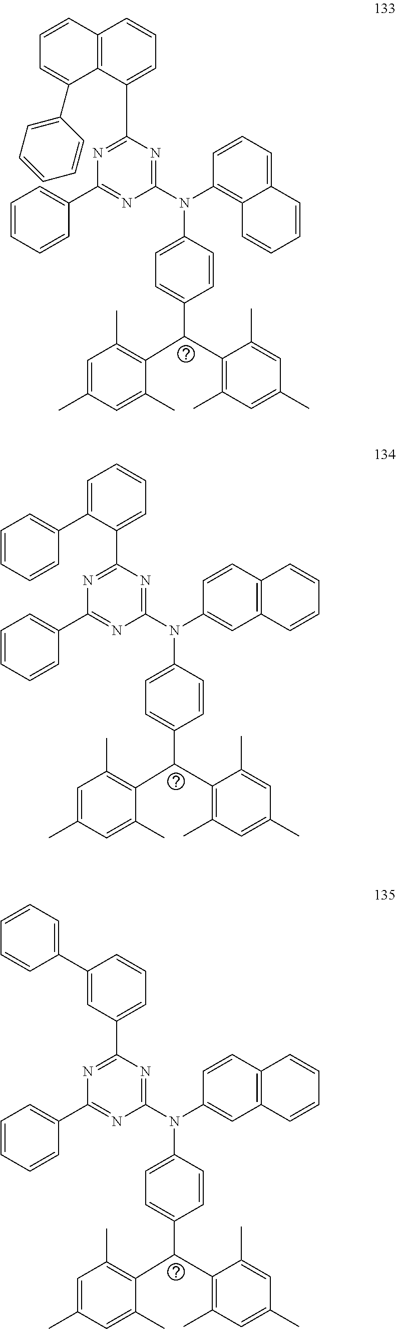 [SMILES: Cc1cc(C)c(C(c2ccc(N(c3nc(-c4ccccc4)nc(-c4cccc5cccc(-c6ccccc6)c45)n3)c3cccc4ccccc34)cc2)c2c(C)cc(C)cc2C)c(C)c1 ; Cc1cc(C)c(C(c2ccc(N(c3ccc4ccccc4c3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cccc(-c5ccccc5)c4)n3)cc2)c2c(C)cc(C)cc2C)c(C)c1]

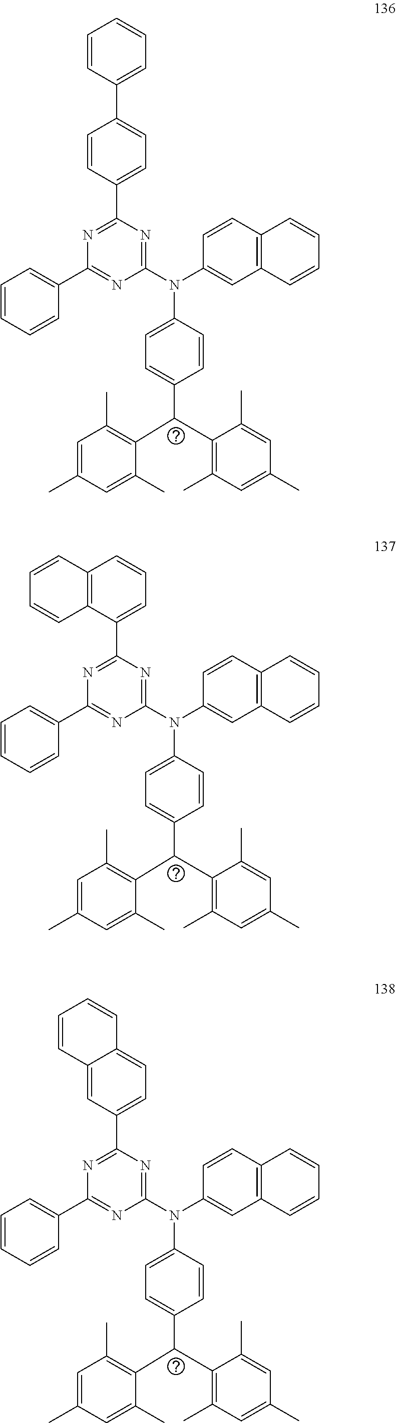 [SMILES: Cc1cc(C)c(C(c2ccc(N(c3ccc4ccccc4c3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cccc5ccccc45)n3)cc2)c2c(C)cc(C)cc2C)c(C)c1 ; Cc1cc(C)c(C(c2ccc(N(c3ccc4ccccc4c3)c3nc(-c4ccccc4)nc(-c4ccc5ccccc5c4)n3)cc2)c2c(C)cc(C)cc2C)c(C)c1]

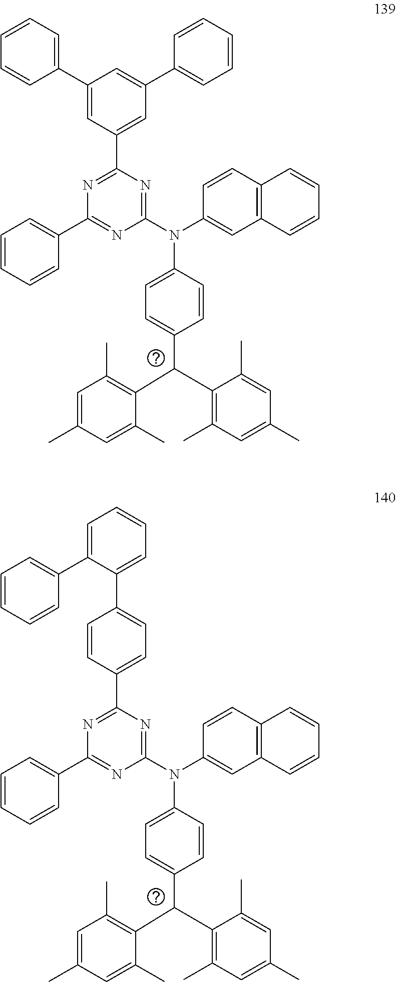 [SMILES: Cc1cc(C)c(C(c2ccc(N(c3ccc4ccccc4c3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc4ccccc4c3)c3nc(-c4ccccc4)nc(-c4ccc(-c5ccccc5-c5ccccc5)cc4)n3)cc2)c2c(C)cc(C)cc2C)c(C)c1]

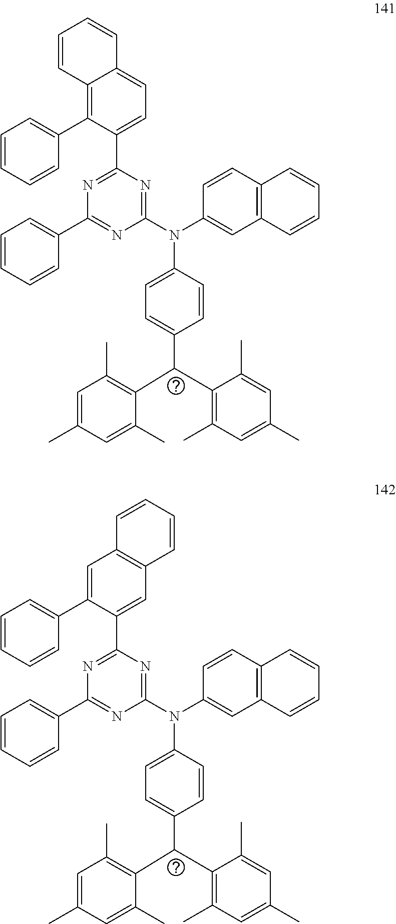 [SMILES: Cc1cc(C)c(C(c2ccc(N(c3ccc4ccccc4c3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cc5ccccc5cc4-c4ccccc4)n3)cc2)c2c(C)cc(C)cc2C)c(C)c1]

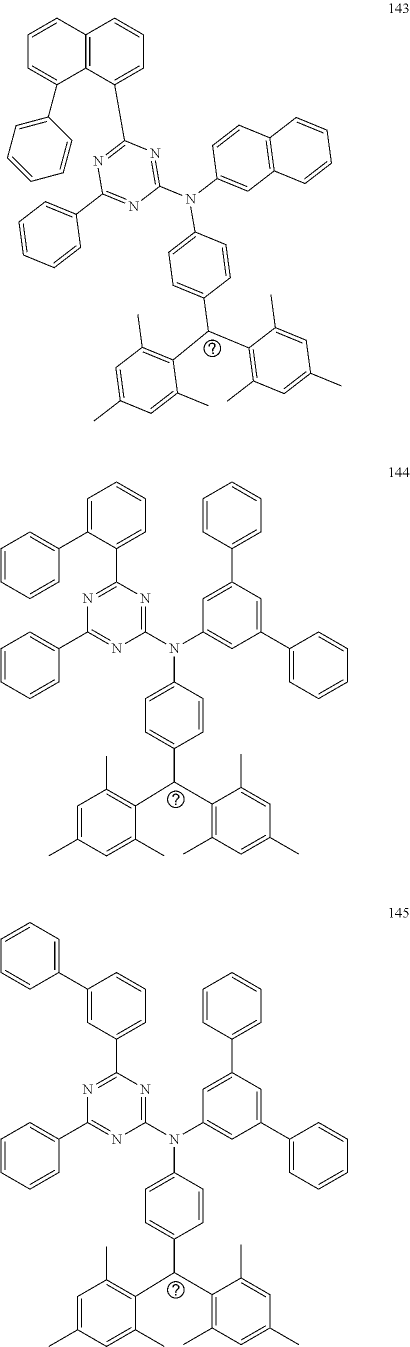 [SMILES: Cc1cc(C)c(C(c2ccc(N(c3ccc4ccccc4c3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4cccc(-c5ccccc5)c4)n3)cc2)c2c(C)cc(C)cc2C)c(C)c1]

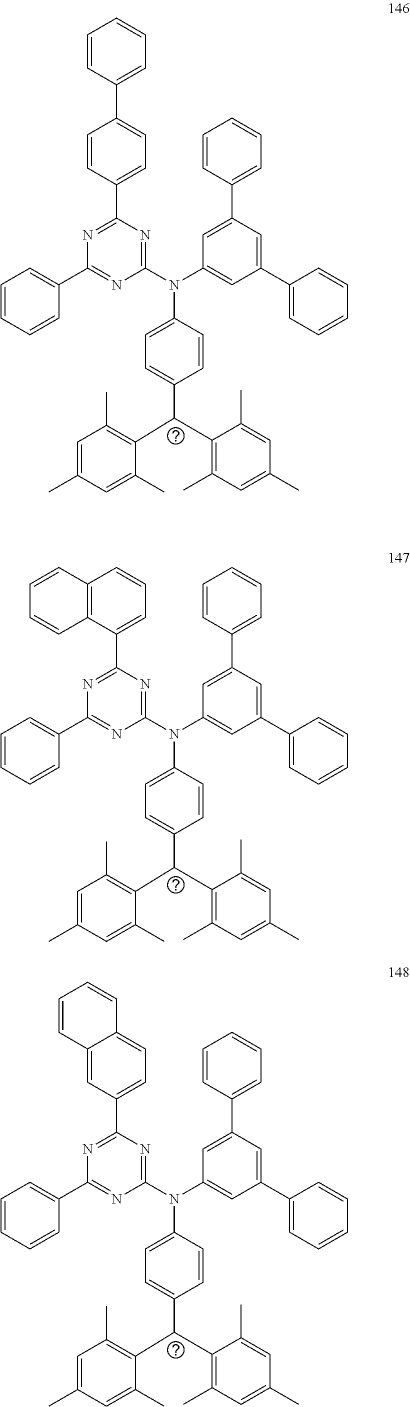

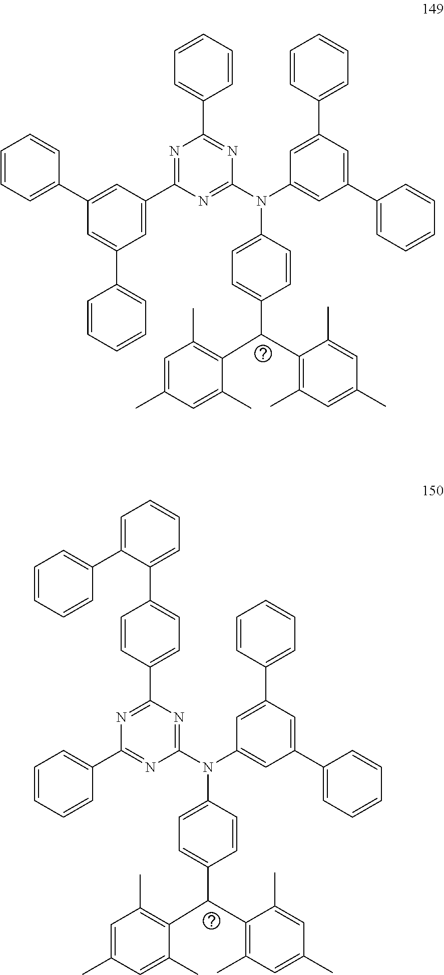 [SMILES: Cc1cc(C)c(C(c2ccc(N(c3cc(-c4ccccc4)cc(-c4ccccc4)c3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4ccc(-c5ccccc5-c5ccccc5)cc4)n3)cc2)c2c(C)cc(C)cc2C)c(C)c1]

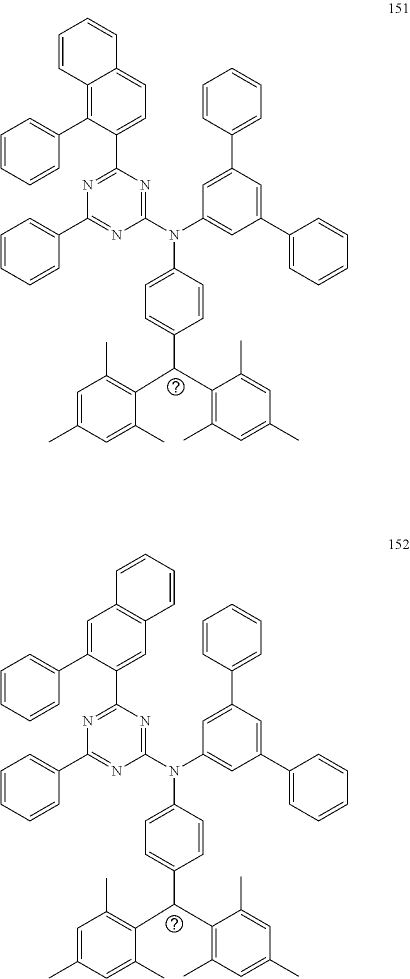 [SMILES: Cc1cc(C)c(C(c2ccc(N(c3cc(-c4ccccc4)cc(-c4ccccc4)c3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4cc5ccccc5cc4-c4ccccc4)n3)cc2)c2c(C)cc(C)cc2C)c(C)c1]

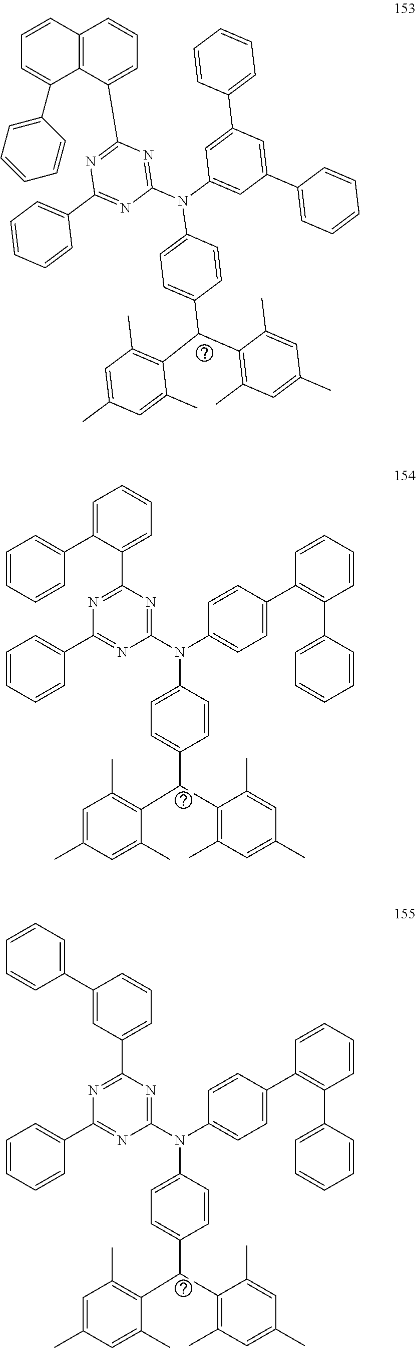 [SMILES: Cc1cc(C)c(C(c2ccc(N(c3cc(-c4ccccc4)cc(-c4ccccc4)c3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cccc(-c5ccccc5)c4)n3)cc2)c2c(C)cc(C)cc2C)c(C)c1]

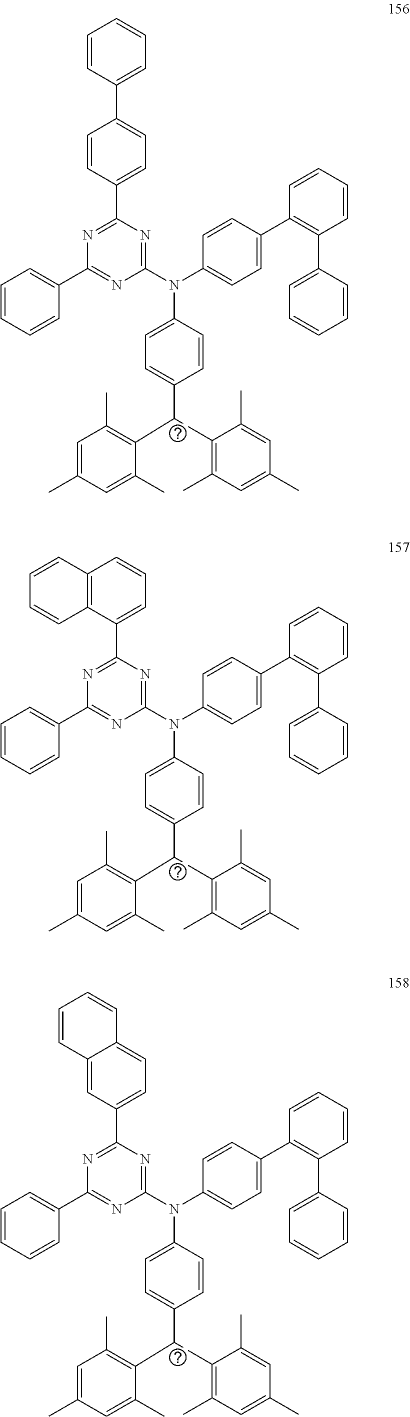 [SMILES: Cc1cc(C)c(C(c2ccc(N(c3ccc(-c4ccccc4-c4ccccc4)cc3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cccc5ccccc45)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccc5ccccc5c4)n3)cc2)c2c(C)cc(C)cc2C)c(C)c1]

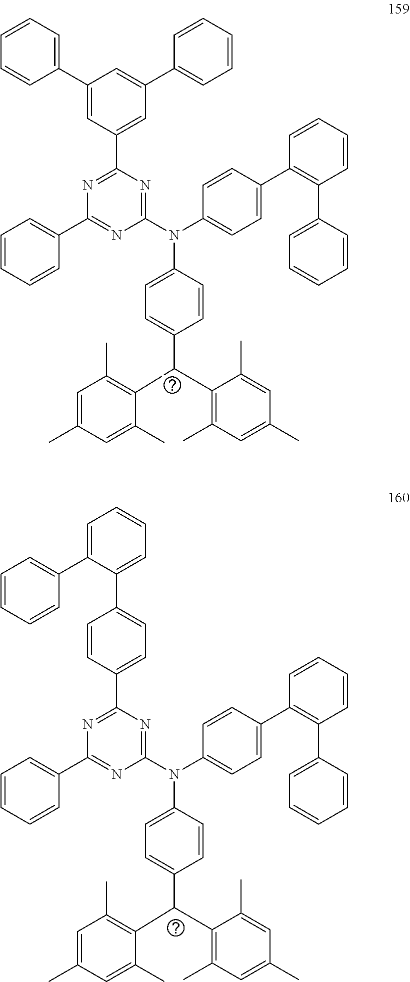 [SMILES: Cc1cc(C)c(C(c2ccc(N(c3ccc(-c4ccccc4-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccc(-c5ccccc5-c5ccccc5)cc4)n3)cc2)c2c(C)cc(C)cc2C)c(C)c1]

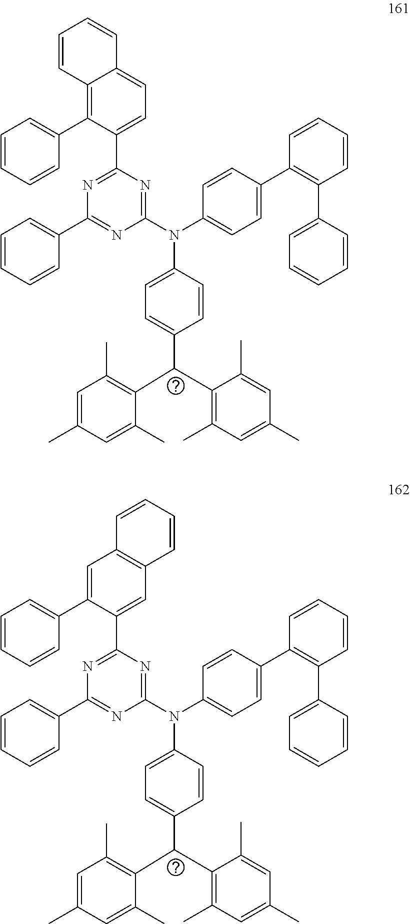 [SMILES: Cc1cc(C)c(C(c2ccc(N(c3ccc(-c4ccccc4-c4ccccc4)cc3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cc5ccccc5cc4-c4ccccc4)n3)cc2)c2c(C)cc(C)cc2C)c(C)c1]

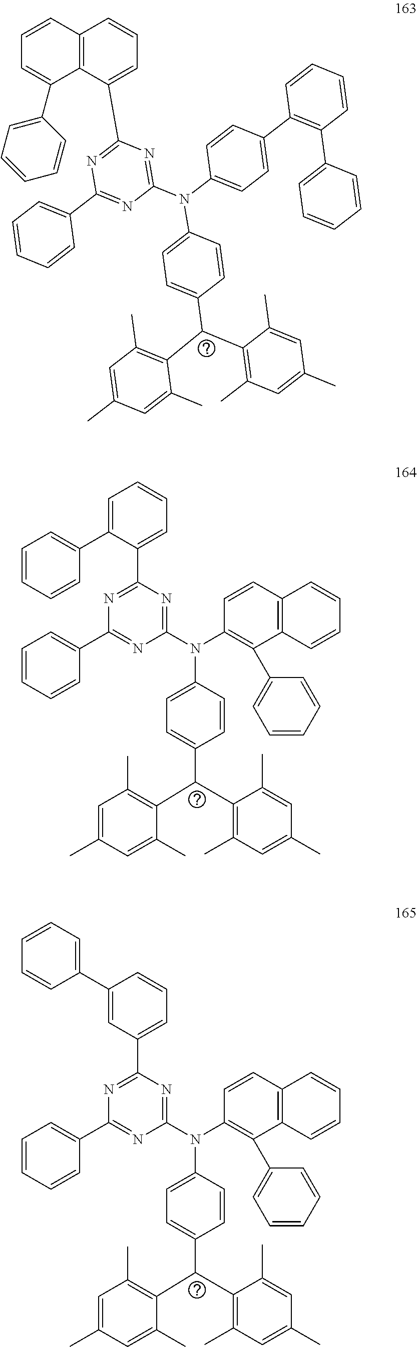 [SMILES: Cc1cc(C)c(C(c2ccc(N(c3ccc(-c4ccccc4-c4ccccc4)cc3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3ccc4ccccc4c3-c3ccccc3)cc2)c2c(C)cc(C)cc2C)c(C)c1]

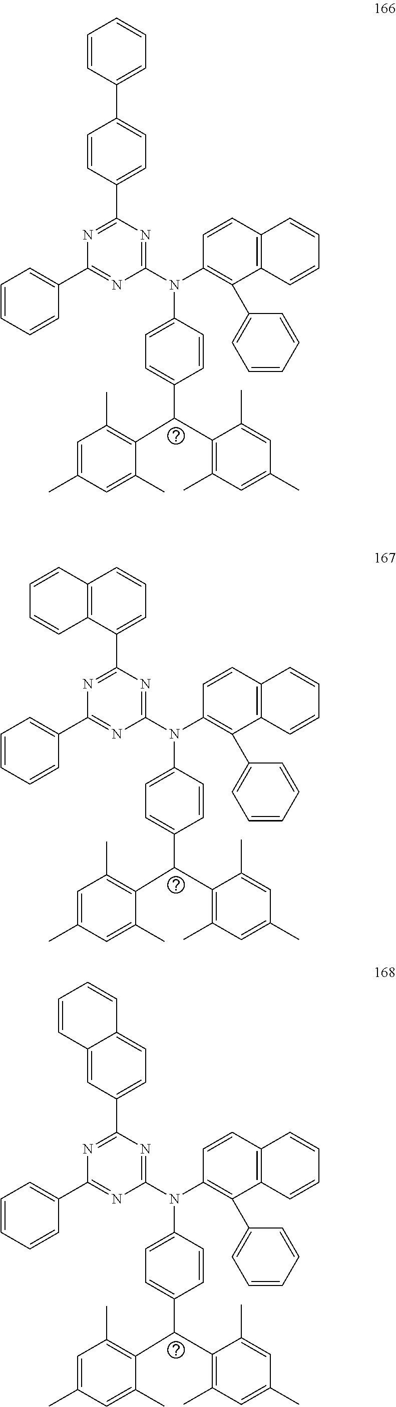

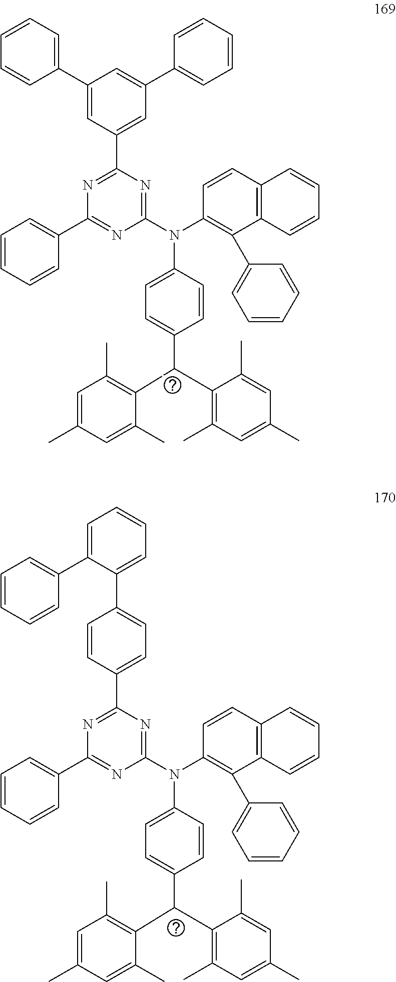 [SMILES: Cc1cc(C)c(C(c2ccc(N(c3nc(-c4ccccc4)nc(-c4cc(-c5ccccc5)cc(-c5ccccc5)c4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3ccc4ccccc4c3-c3ccccc3)cc2)c2c(C)cc(C)cc2C)c(C)c1]

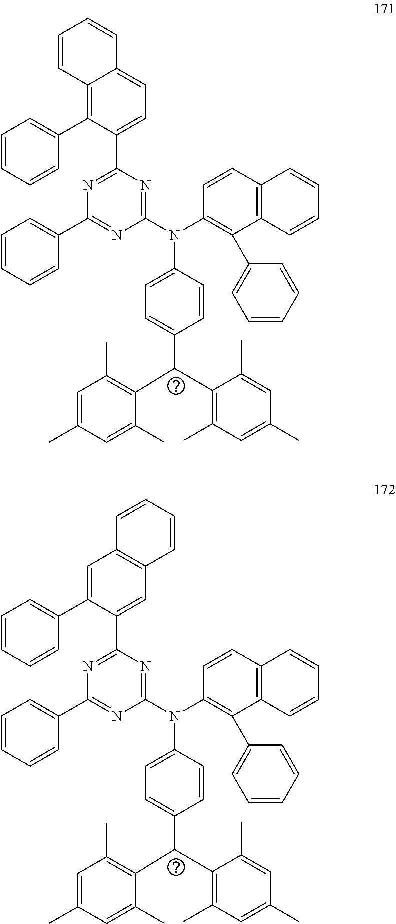 [SMILES: Cc1cc(C)c(C(c2ccc(N(c3nc(-c4ccccc4)nc(-c4ccc5ccccc5c4-c4ccccc4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3ccc4ccccc4c3-c3ccccc3)cc2)c2c(C)cc(C)cc2C)c(C)c1]

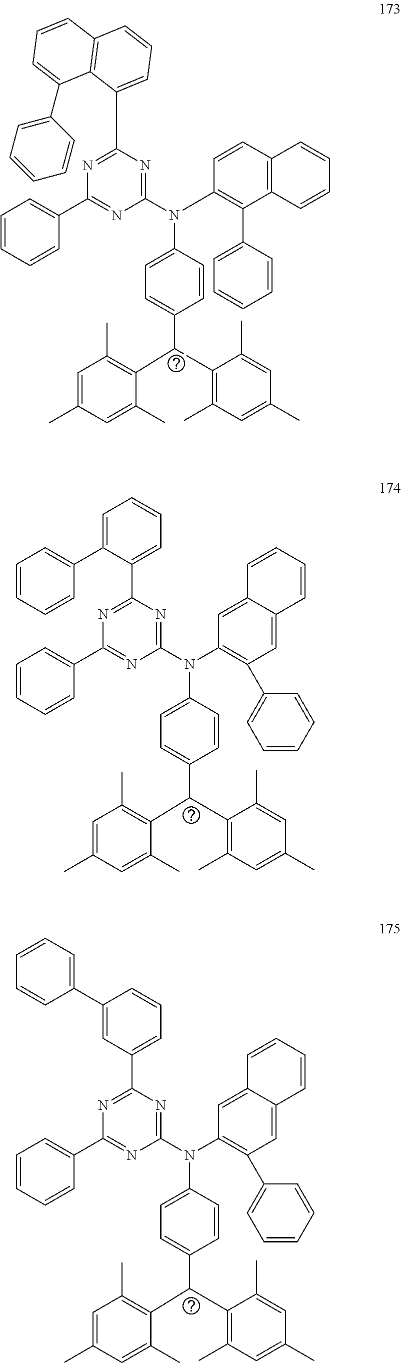 [SMILES: Cc1cc(C)c(C(c2ccc(N(c3nc(-c4ccccc4)nc(-c4cccc5cccc(-c6ccccc6)c45)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3cc4ccccc4cc3-c3ccccc3)cc2)c2c(C)cc(C)cc2C)c(C)c1]

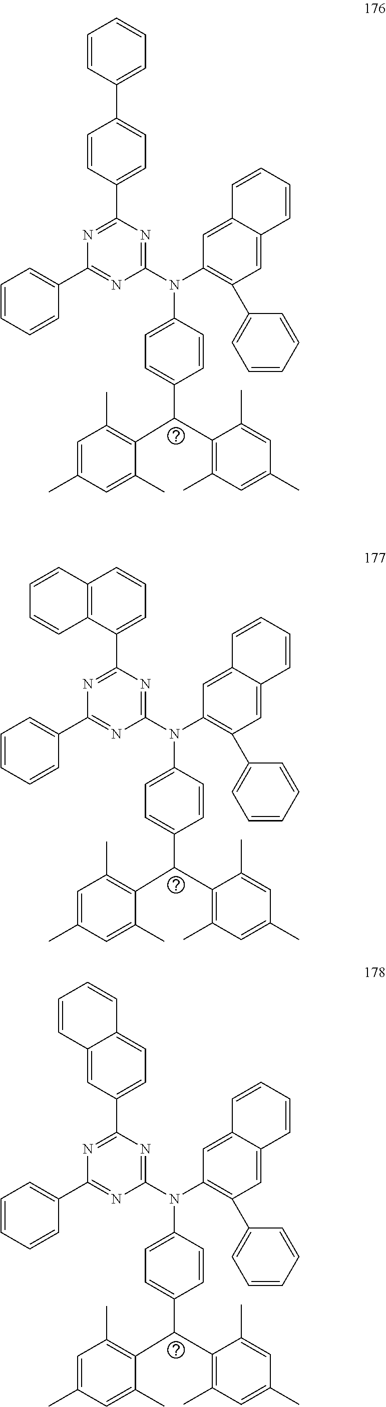

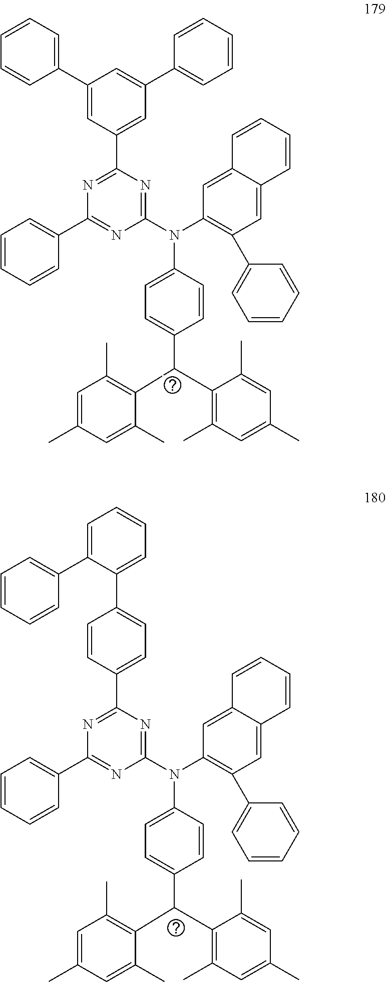 [SMILES: Cc1cc(C)c(C(c2ccc(N(c3nc(-c4ccccc4)nc(-c4cc(-c5ccccc5)cc(-c5ccccc5)c4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3cc4ccccc4cc3-c3ccccc3)cc2)c2c(C)cc(C)cc2C)c(C)c1]

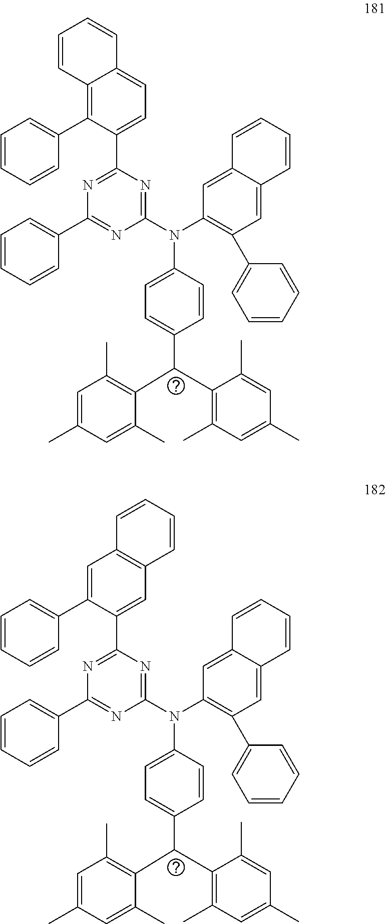 [SMILES: Cc1cc(C)c(C(c2ccc(N(c3nc(-c4ccccc4)nc(-c4ccc5ccccc5c4-c4ccccc4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3cc4ccccc4cc3-c3ccccc3)cc2)c2c(C)cc(C)cc2C)c(C)c1]

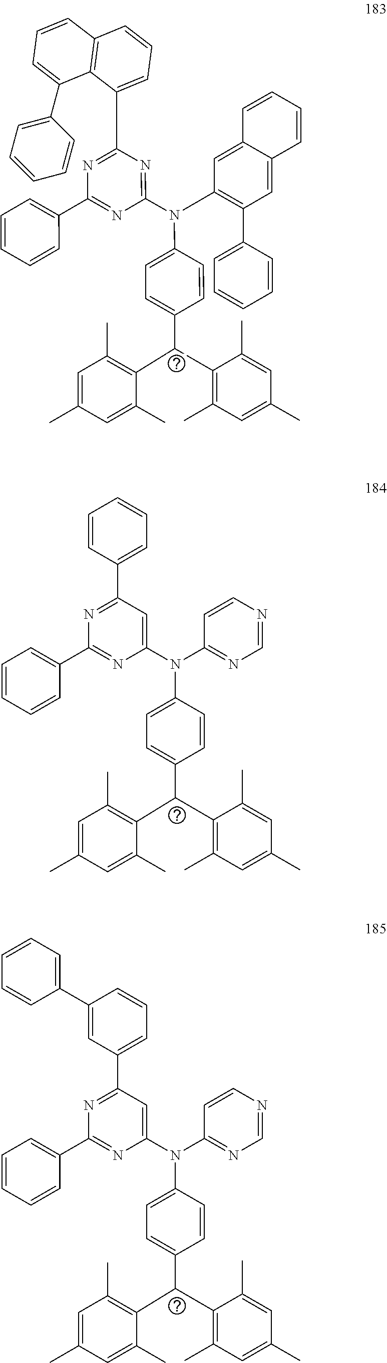 [SMILES: Cc1cc(C)c(C(c2ccc(N(c3nc(-c4ccccc4)nc(-c4cccc5cccc(-c6ccccc6)c45)n3)c3cc4ccccc4cc3-c3ccccc3)cc2)c2c(C)cc(C)cc2C)c(C)c1 ; Cc1cc(C)c(C(c2ccc(N(c3ccncn3)c3cc(-c4ccccc4)nc(-c4ccccc4)n3)cc2)c2c(C)cc(C)cc2C)c(C)c1 ; Cc1cc(C)c(C(c2ccc(N(c3ccncn3)c3cc(-c4cccc(-c5ccccc5)c4)nc(-c4ccccc4)n3)cc2)c2c(C)cc(C)cc2C)c(C)c1]

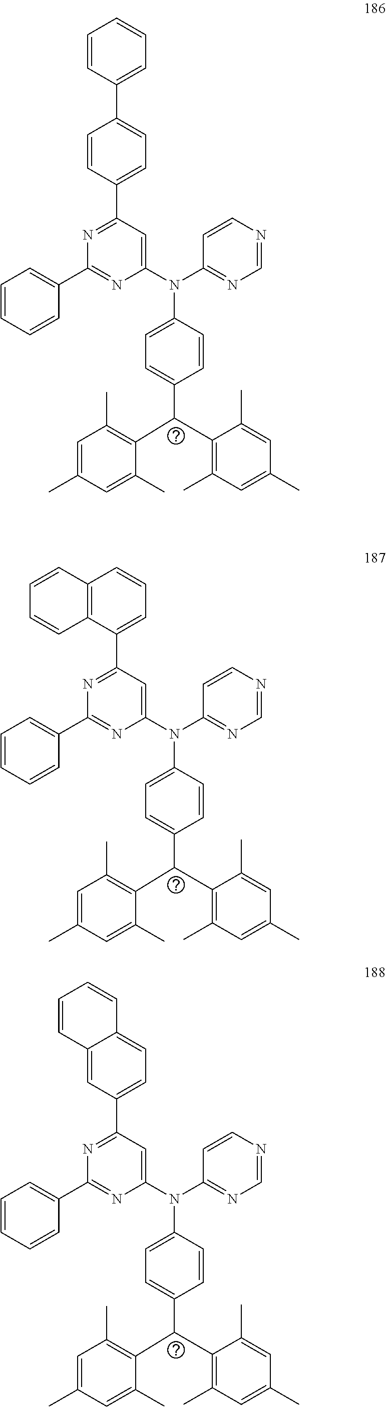

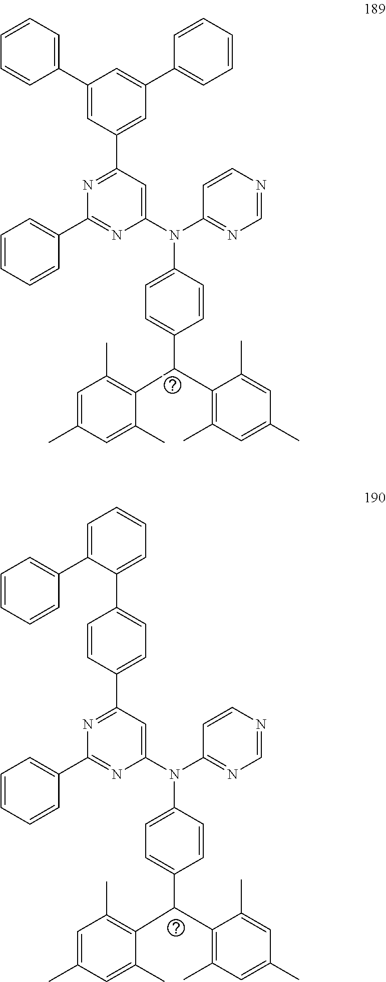 [SMILES: Cc1cc(C)c(C(c2ccc(N(c3ccncn3)c3cc(-c4cc(-c5ccccc5)cc(-c5ccccc5)c4)nc(-c4ccccc4)n3)cc2)c2c(C)cc(C)cc2C)c(C)c1 ; Cc1cc(C)c(C(c2ccc(N(c3ccncn3)c3cc(-c4ccc(-c5ccccc5-c5ccccc5)cc4)nc(-c4ccccc4)n3)cc2)c2c(C)cc(C)cc2C)c(C)c1]

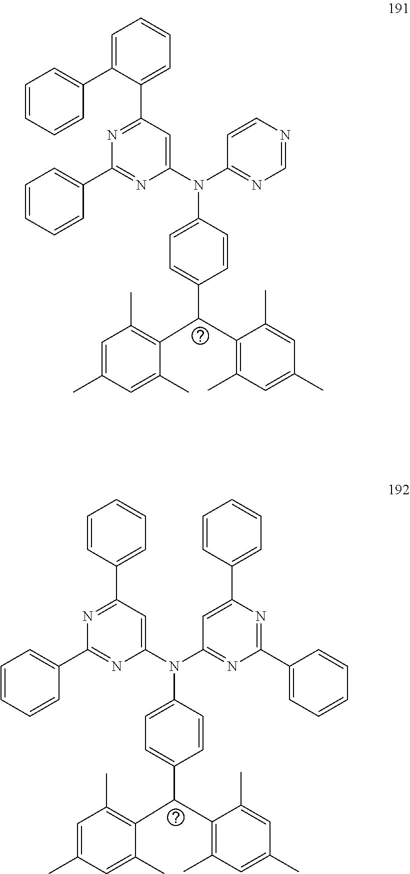 [SMILES: Cc1cc(C)c(C(c2ccc(N(c3ccncn3)c3cc(-c4ccccc4-c4ccccc4)nc(-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccccc4)nc(-c4ccccc4)n3)cc2)c2c(C)cc(C)cc2C)c(C)c1]

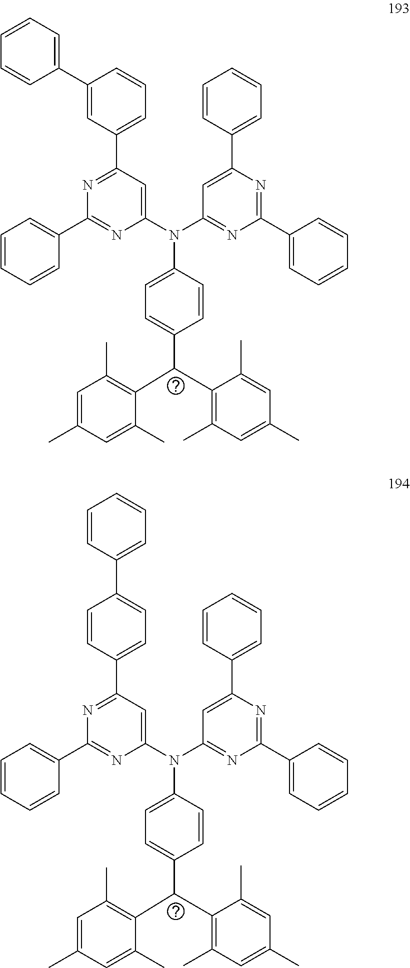 [SMILES: Cc1cc(C)c(C(c2ccc(N(c3cc(-c4ccccc4)nc(-c4ccccc4)n3)c3cc(-c4cccc(-c5ccccc5)c4)nc(-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccc(-c5ccccc5)cc4)nc(-c4ccccc4)n3)cc2)c2c(C)cc(C)cc2C)c(C)c1]

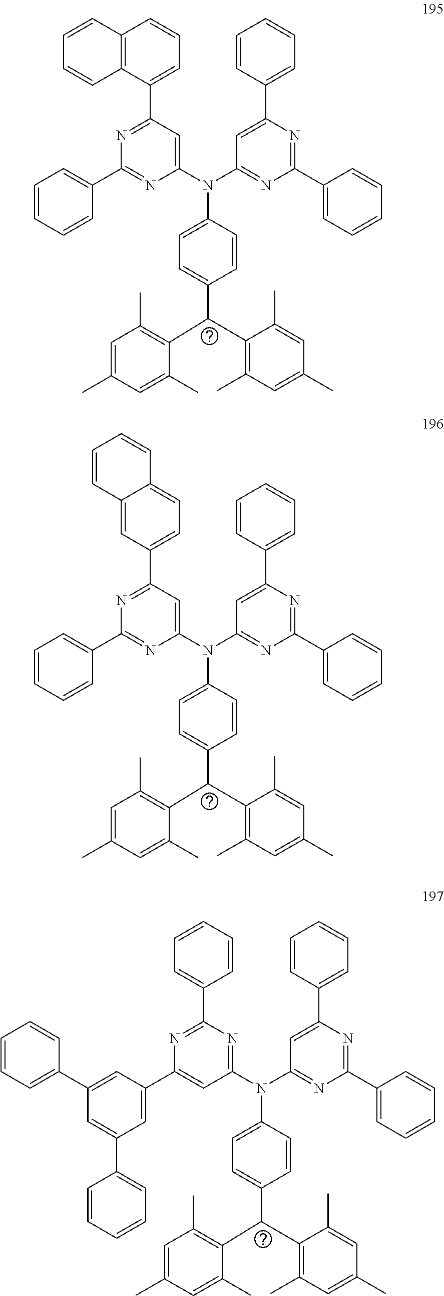

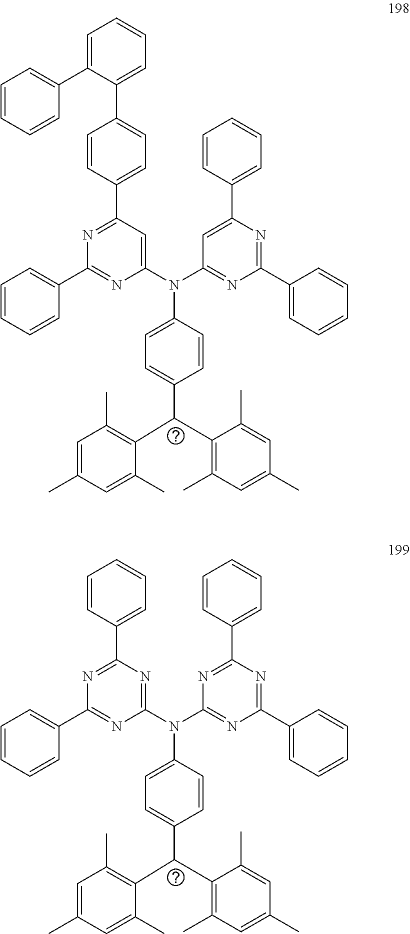 [SMILES: Cc1cc(C)c(C(c2ccc(N(c3cc(-c4ccccc4)nc(-c4ccccc4)n3)c3cc(-c4ccc(-c5ccccc5-c5ccccc5)cc4)nc(-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccccc4)n3)cc2)c2c(C)cc(C)cc2C)c(C)c1]

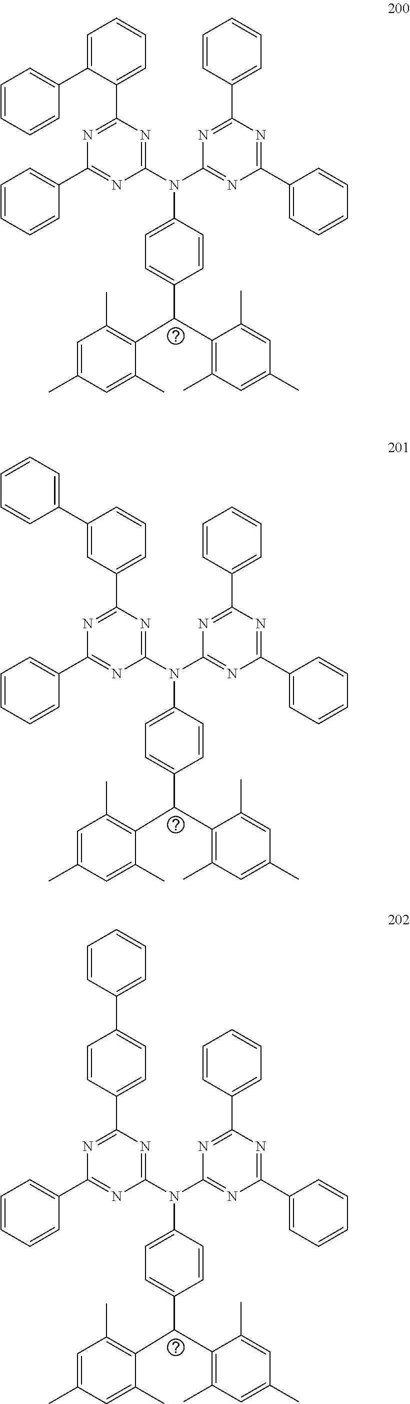 [SMILES: Cc1cc(C)c(C(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc(-c5ccccc5)cc4)n3)cc2)c2c(C)cc(C)cc2C)c(C)c1]

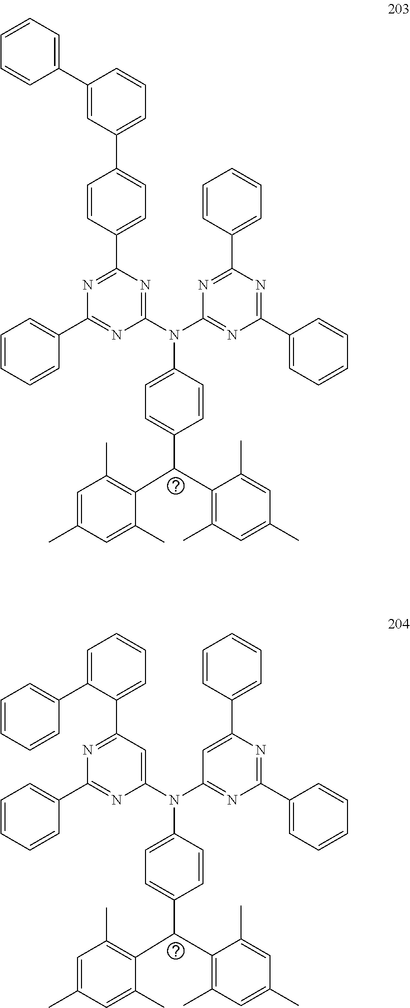 [SMILES: Cc1cc(C)c(C(c2ccc(N(c3nc(-c4ccccc4)nc(-c4ccccc4)n3)c3nc(-c4ccccc4)nc(-c4ccc(-c5cccc(-c6ccccc6)c5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccccc4-c4ccccc4)nc(-c4ccccc4)n3)cc2)c2c(C)cc(C)cc2C)c(C)c1]

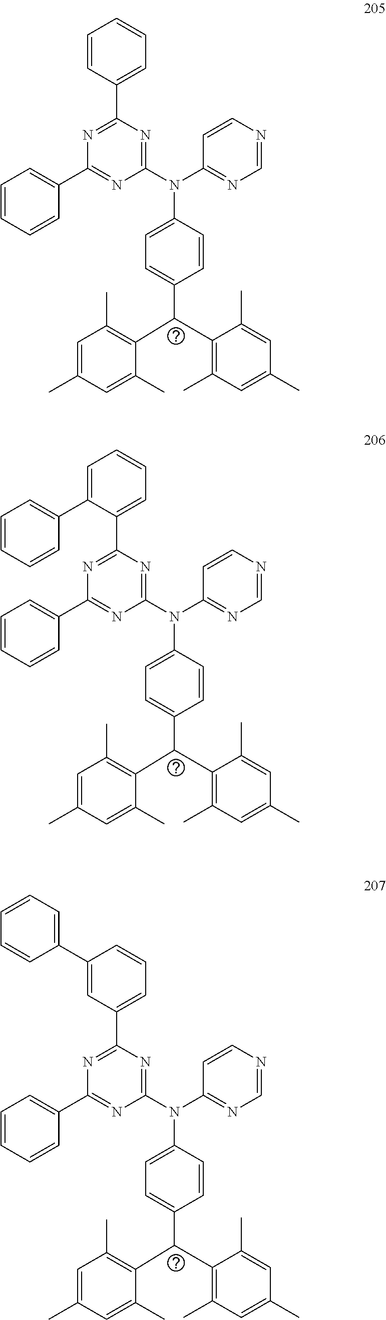 [SMILES: Cc1cc(C)c(C(c2ccc(N(c3ccncn3)c3nc(-c4ccccc4)nc(-c4ccccc4)n3)cc2)c2c(C)cc(C)cc2C)c(C)c1 ; Cc1cc(C)c(C(c2ccc(N(c3ccncn3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccncn3)c3nc(-c4ccccc4)nc(-c4cccc(-c5ccccc5)c4)n3)cc2)c2c(C)cc(C)cc2C)c(C)c1]

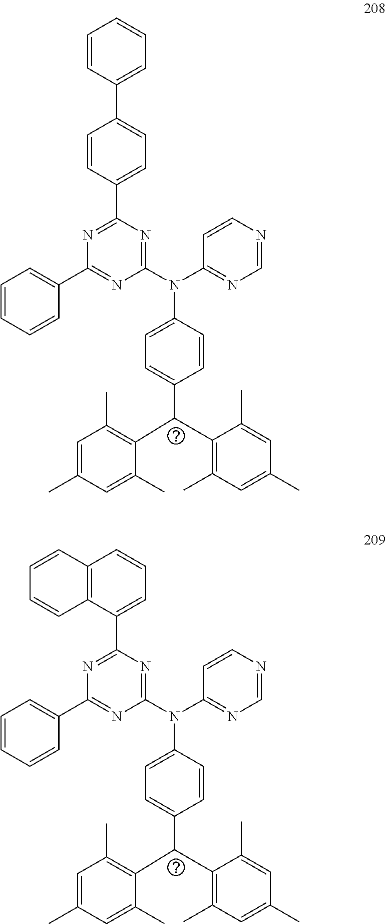 [SMILES: Cc1cc(C)c(C(c2ccc(N(c3ccncn3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccncn3)c3nc(-c4ccccc4)nc(-c4cccc5ccccc45)n3)cc2)c2c(C)cc(C)cc2C)c(C)c1]

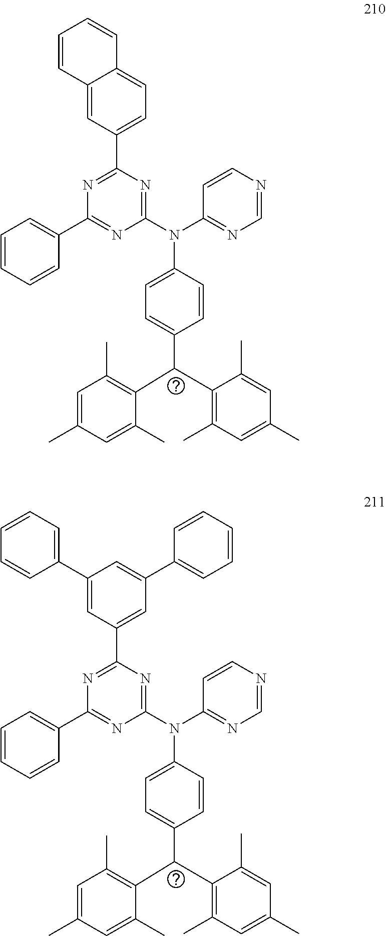 [SMILES: Cc1cc(C)c(C(c2ccc(N(c3ccncn3)c3nc(-c4ccccc4)nc(-c4ccc5ccccc5c4)n3)cc2)c2c(C)cc(C)cc2C)c(C)c1 ; Cc1cc(C)c(C(c2ccc(N(c3ccncn3)c3nc(-c4ccccc4)nc(-c4cc(-c5ccccc5)cc(-c5ccccc5)c4)n3)cc2)c2c(C)cc(C)cc2C)c(C)c1]

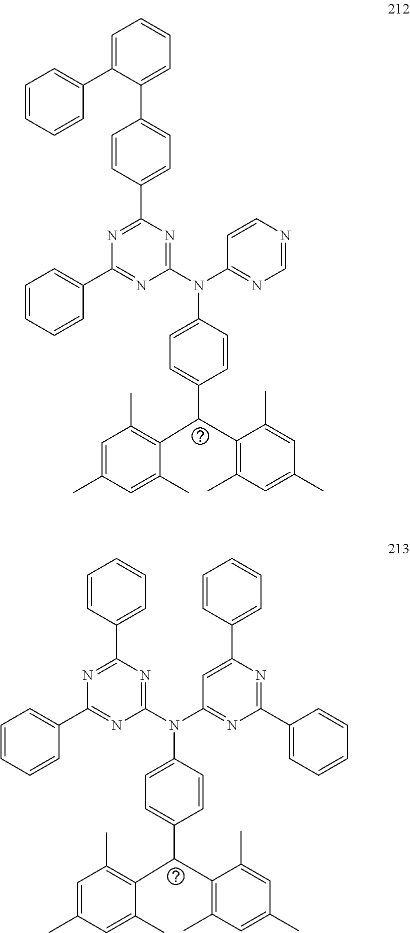 [SMILES: Cc1cc(C)c(C(c2ccc(N(c3ccncn3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccccc4)n3)cc2)c2c(C)cc(C)cc2C)c(C)c1]

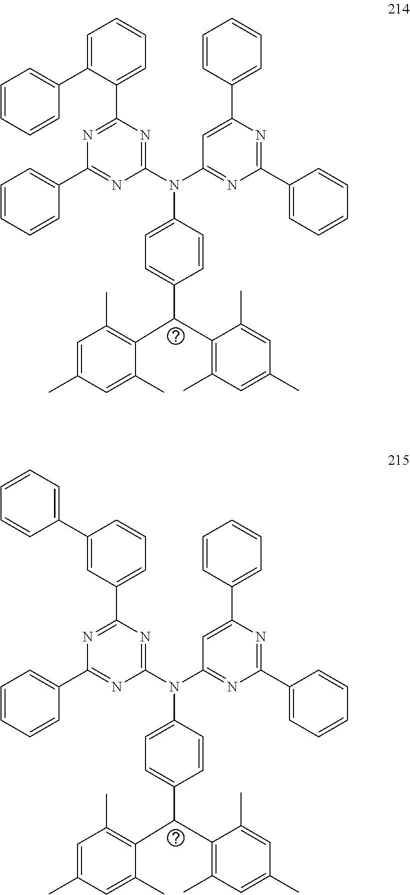 [SMILES: Cc1cc(C)c(C(c2ccc(N(c3cc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1]

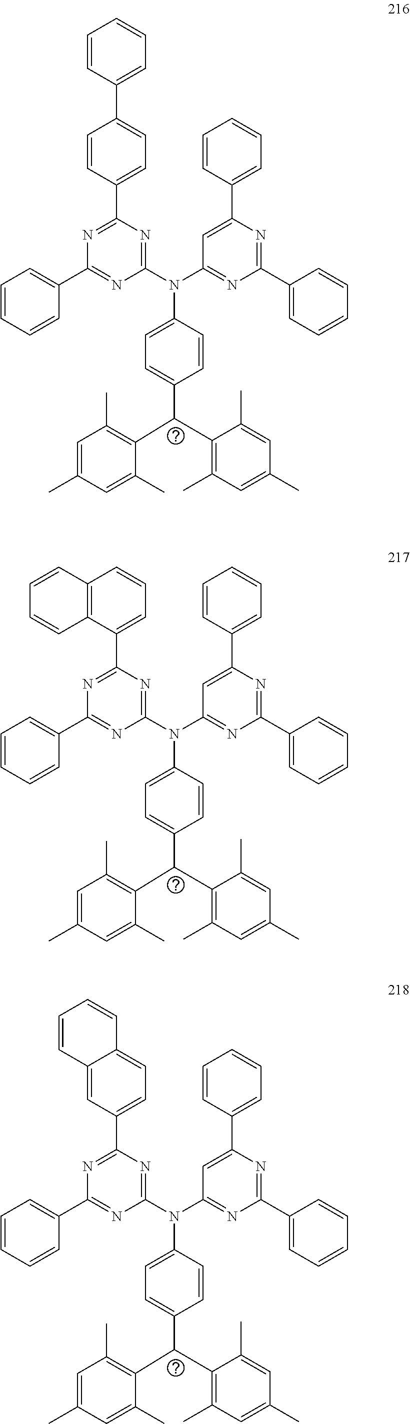 [SMILES: Cc1cc(C)c(C(c2ccc(N(c3cc(-c4ccccc4)nc(-c4ccccc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccc5ccccc5c4)n3)cc2)c2c(C)cc(C)cc2C)c(C)c1]

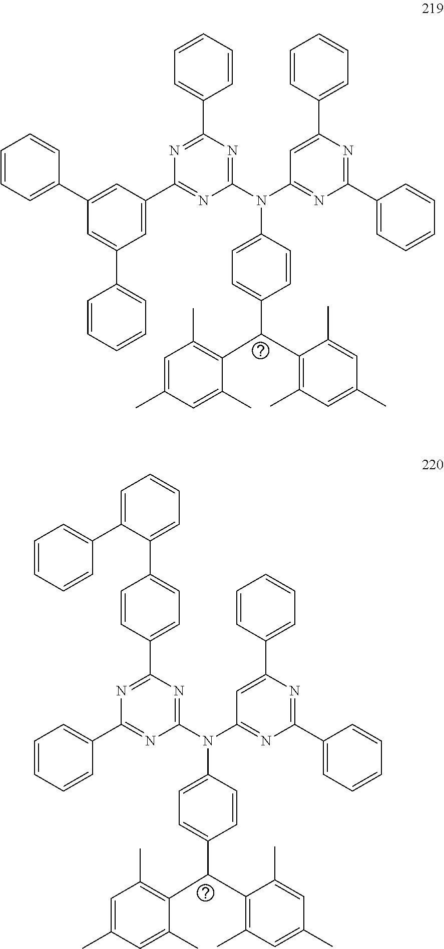 [SMILES: Cc1cc(C)c(C(c2ccc(N(c3cc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1]

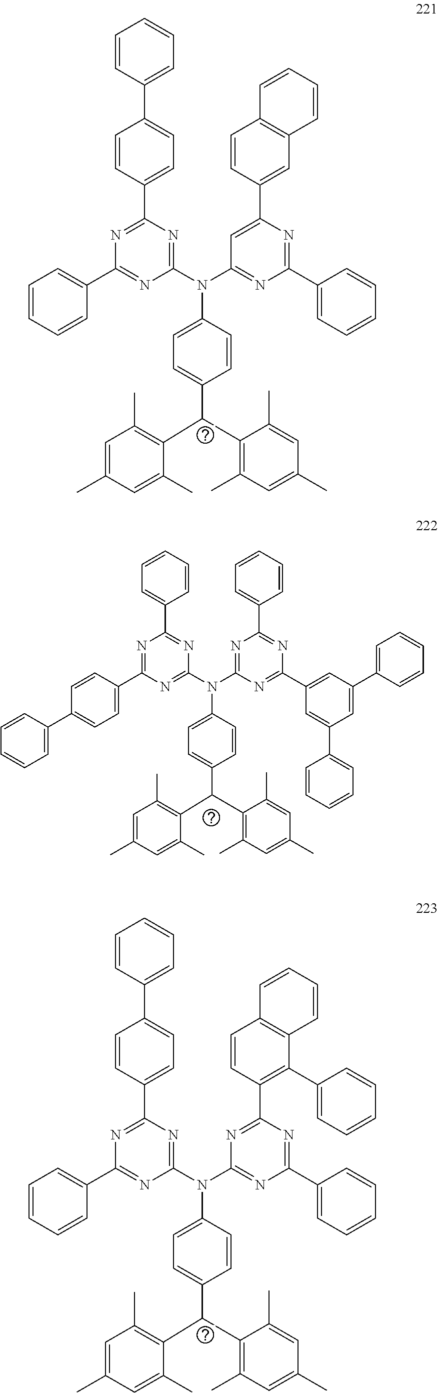 [SMILES: Cc1cc(C)c(C(c2ccc(N(c3cc(-c4ccc5ccccc5c4)nc(-c4ccccc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4ccc5ccccc5c4-c4ccccc4)n3)cc2)c2c(C)cc(C)cc2C)c(C)c1]

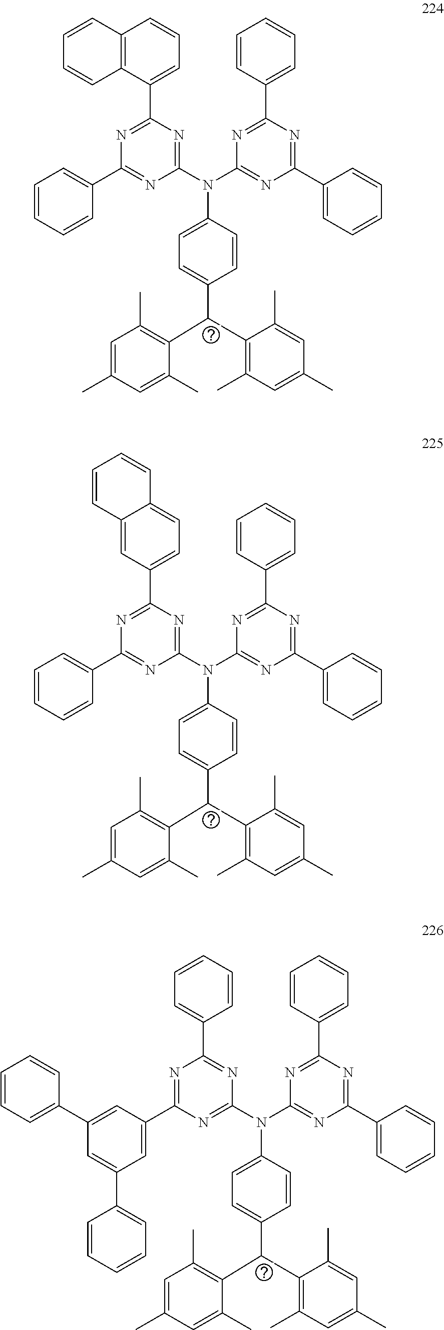 [SMILES: Cc1cc(C)c(C(c2ccc(N(c3nc(-c4ccccc4)nc(-c4ccccc4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc5ccccc5c4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1]

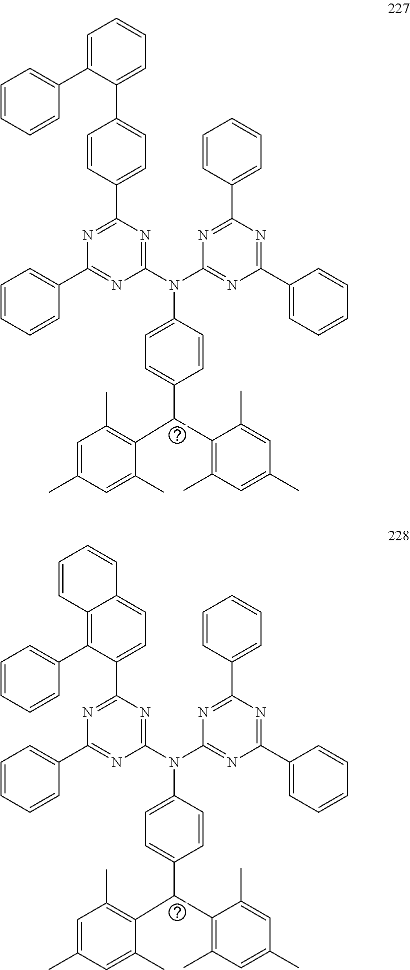 [SMILES: Cc1cc(C)c(C(c2ccc(N(c3nc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc5ccccc5c4-c4ccccc4)n3)cc2)c2c(C)cc(C)cc2C)c(C)c1]

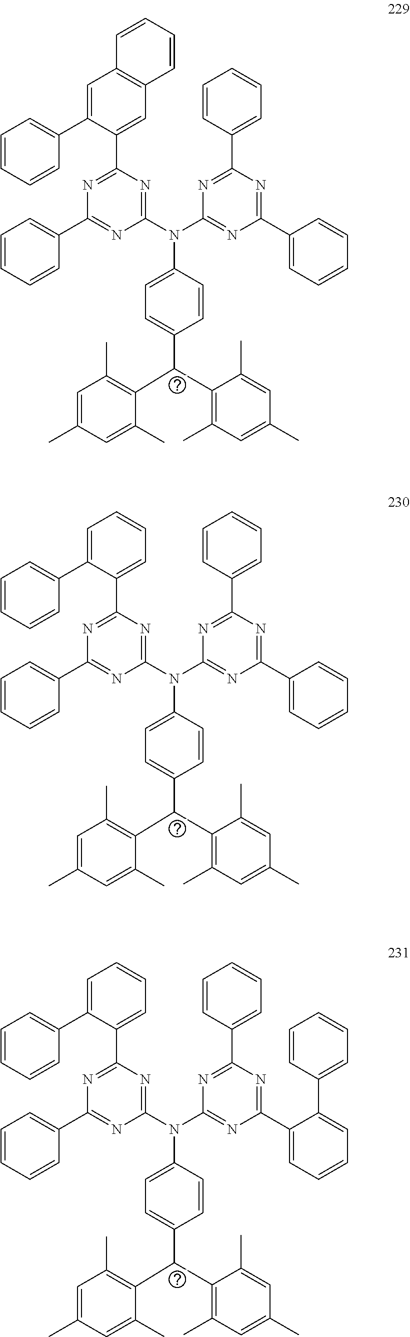 [SMILES: Cc1cc(C)c(C(c2ccc(N(c3nc(-c4ccccc4)nc(-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)cc(C)cc2C)c(C)c1]

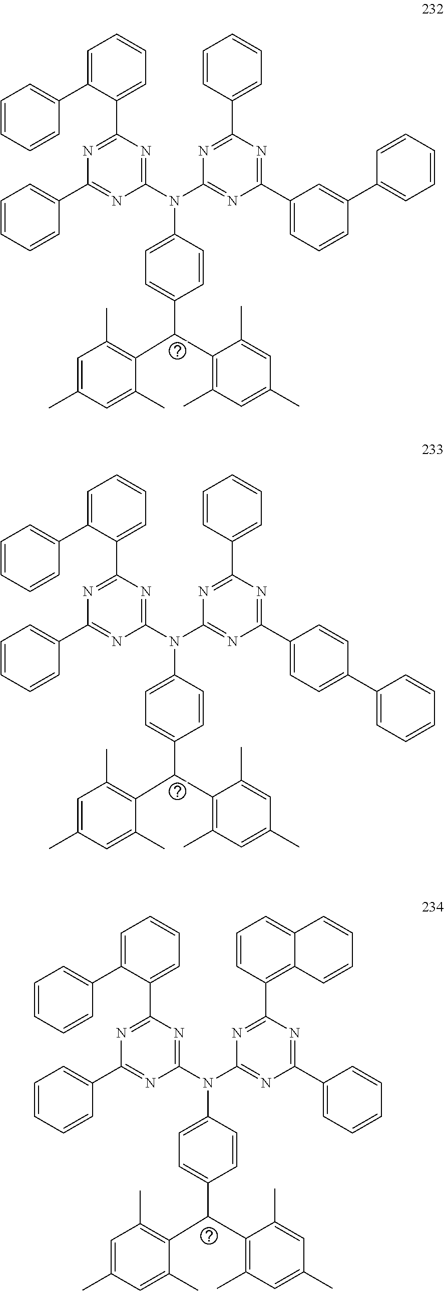 [SMILES: Cc1cc(C)c(C(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4cccc5ccccc45)n3)cc2)c2c(C)cc(C)cc2C)c(C)c1]

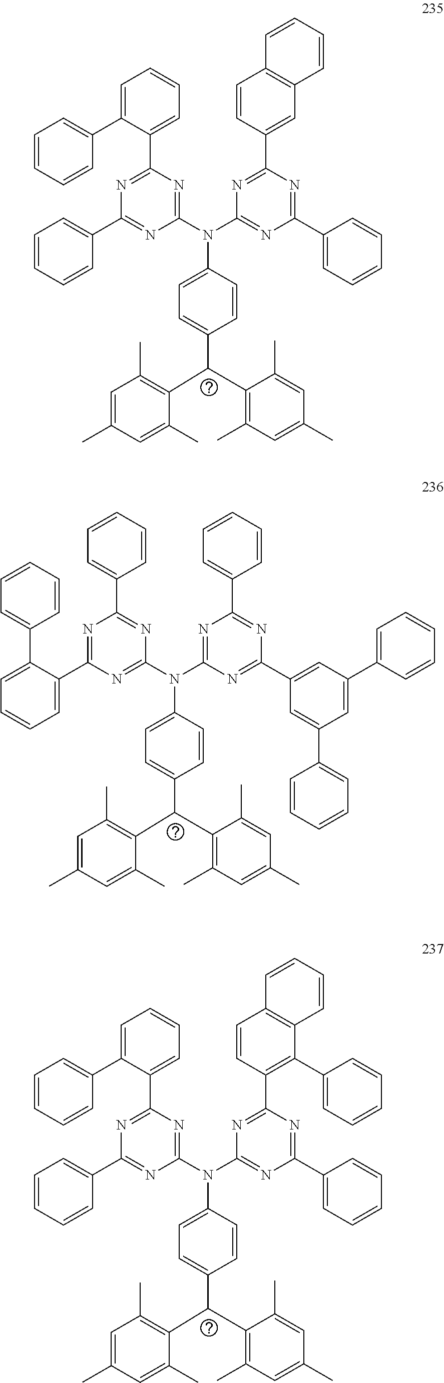 [SMILES: Cc1cc(C)c(C(c2ccc(N(c3nc(-c4ccccc4)nc(-c4ccc5ccccc5c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4ccc5ccccc5c4-c4ccccc4)n3)cc2)c2c(C)cc(C)cc2C)c(C)c1]

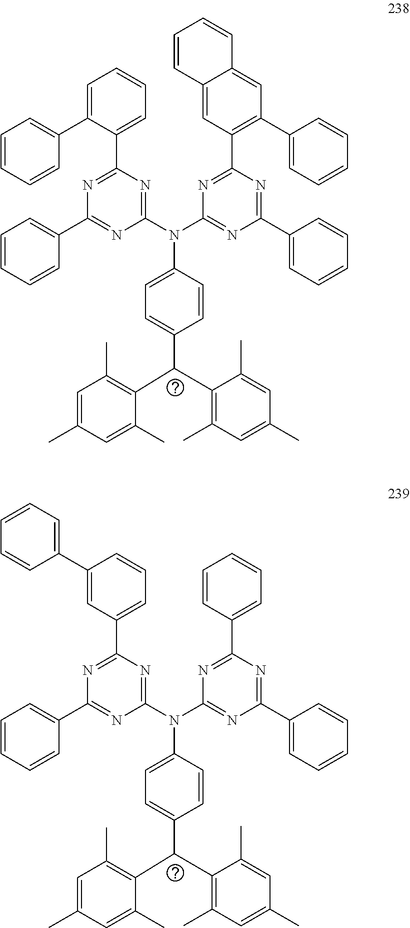 [SMILES: Cc1cc(C)c(C(c2ccc(N(c3nc(-c4ccccc4)nc(-c4ccccc4-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1]

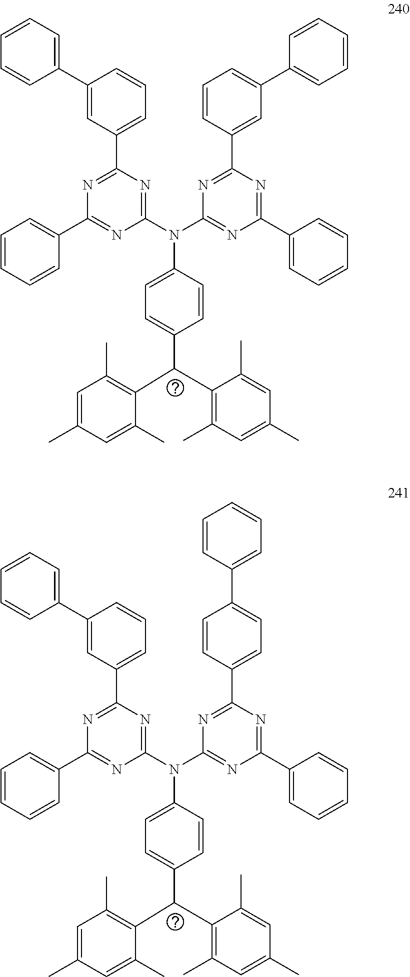 [SMILES: Cc1cc(C)c(C(c2ccc(N(c3nc(-c4ccccc4)nc(-c4cccc(-c5ccccc5)c4)n3)c3nc(-c4ccccc4)nc(-c4cccc(-c5ccccc5)c4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cccc(-c5ccccc5)c4)n3)cc2)c2c(C)cc(C)cc2C)c(C)c1]

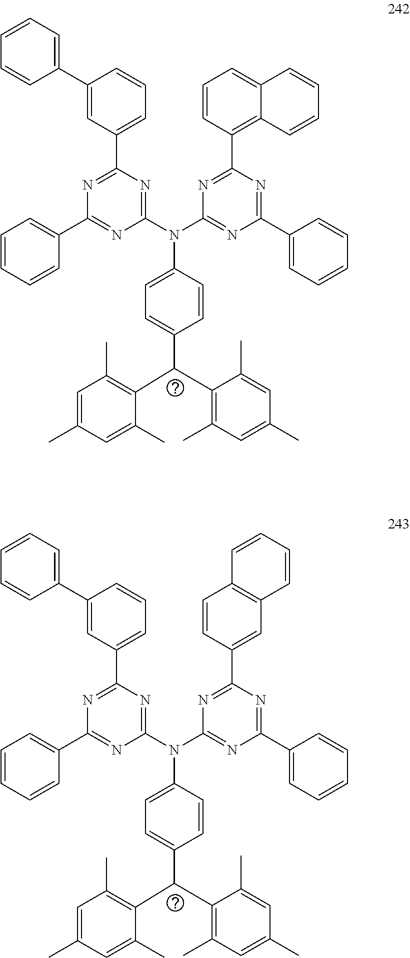 [SMILES: Cc1cc(C)c(C(c2ccc(N(c3nc(-c4ccccc4)nc(-c4cccc(-c5ccccc5)c4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4ccc5ccccc5c4)n3)cc2)c2c(C)cc(C)cc2C)c(C)c1]

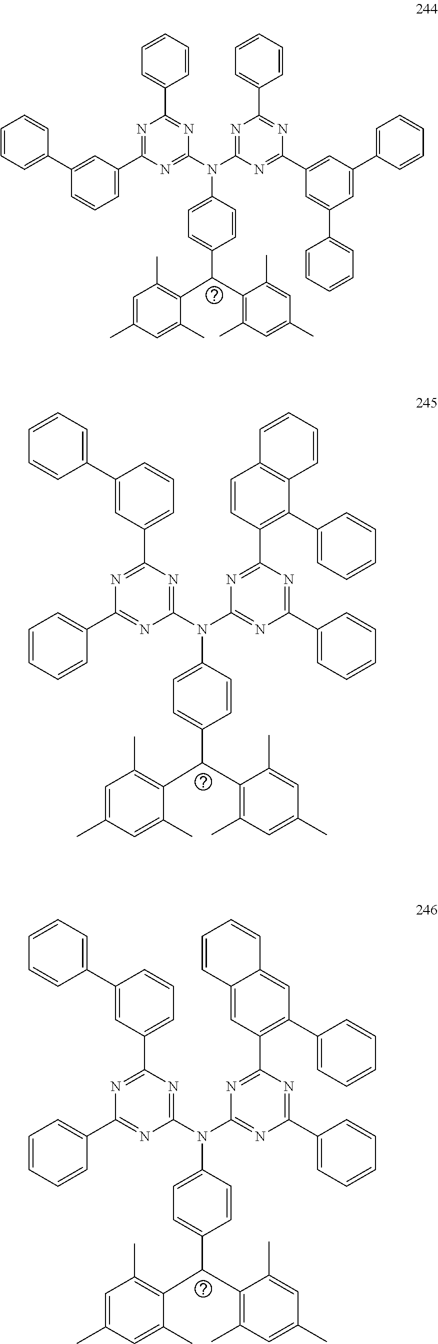 [SMILES: Cc1cc(C)c(C(c2ccc(N(c3nc(-c4ccccc4)nc(-c4cccc(-c5ccccc5)c4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4cc5ccccc5cc4-c4ccccc4)n3)cc2)c2c(C)cc(C)cc2C)c(C)c1]

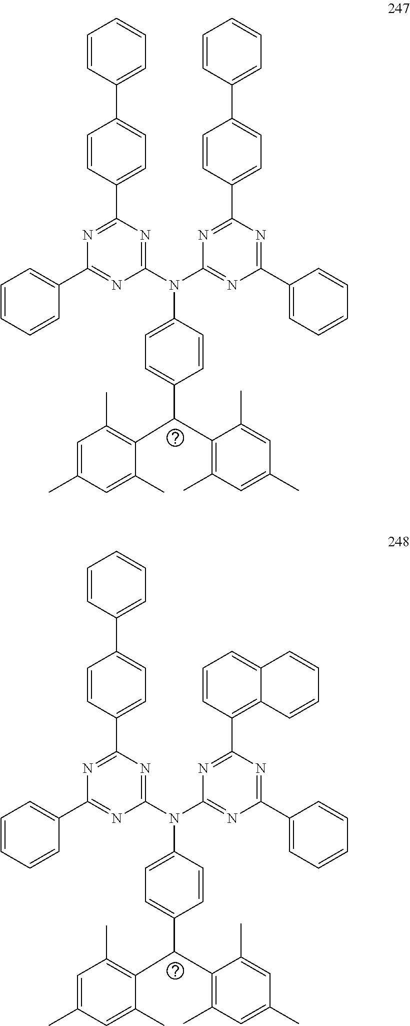 [SMILES: Cc1cc(C)c(C(c2ccc(N(c3nc(-c4ccccc4)nc(-c4ccc(-c5ccccc5)cc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cccc5ccccc45)n3)cc2)c2c(C)cc(C)cc2C)c(C)c1]

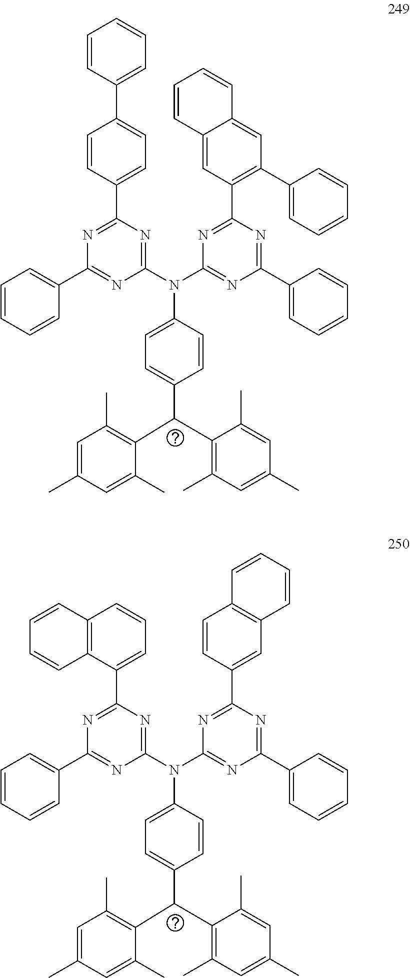 [SMILES: Cc1cc(C)c(C(c2ccc(N(c3nc(-c4ccccc4)nc(-c4ccc(-c5ccccc5)cc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccc5ccccc5c4)n3)c3nc(-c4ccccc4)nc(-c4cccc5ccccc45)n3)cc2)c2c(C)cc(C)cc2C)c(C)c1]

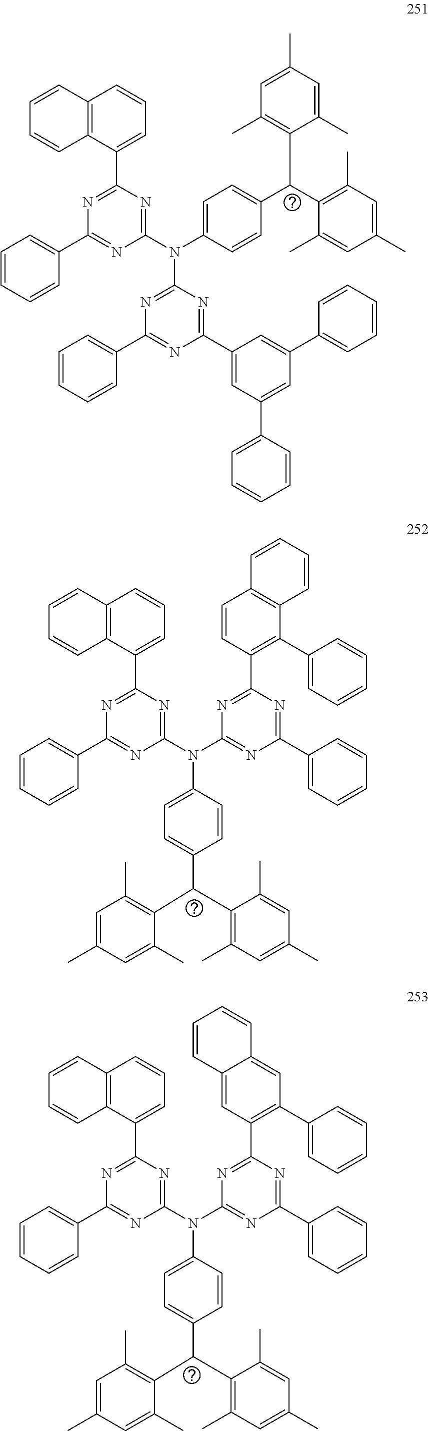

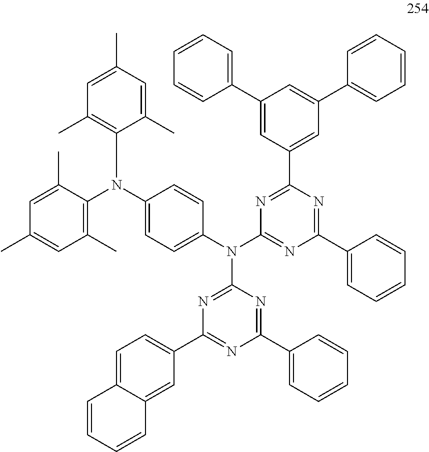 [SMILES: Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4)n3)cc2)c2c(C)cc(C)cc2C)c(C)c1]

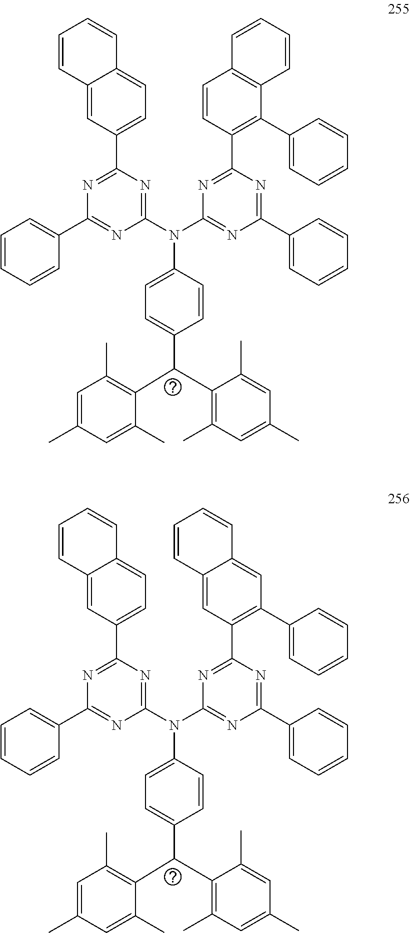 [SMILES: Cc1cc(C)c(C(c2ccc(N(c3nc(-c4ccccc4)nc(-c4ccc5ccccc5c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4ccc5ccccc5c4)n3)c3nc(-c4ccccc4)nc(-c4cc5ccccc5cc4-c4ccccc4)n3)cc2)c2c(C)cc(C)cc2C)c(C)c1]

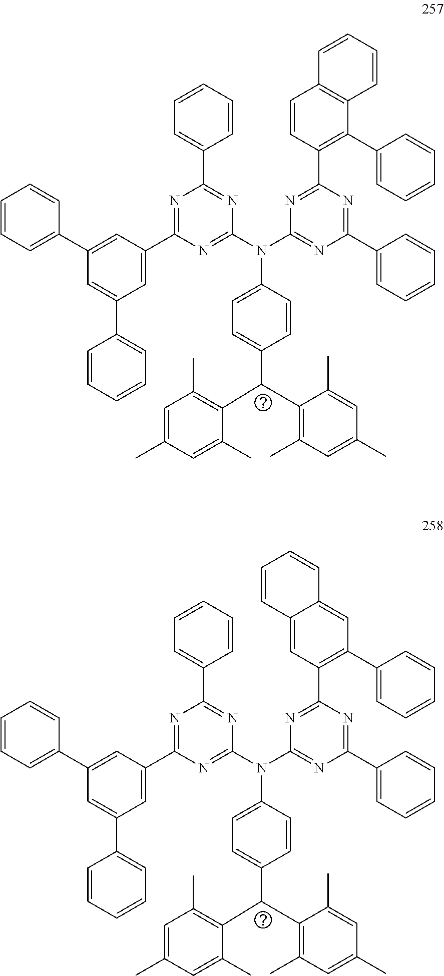 [SMILES: Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4cc5ccccc5cc4-c4ccccc4)n3)cc2)c2c(C)cc(C)cc2C)c(C)c1]

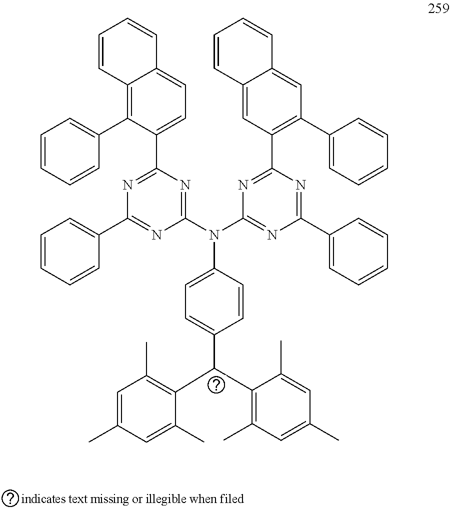 [SMILES: Cc1cc(C)c(C(c2ccc(N(c3nc(-c4ccccc4)nc(-c4cc5ccccc5cc4-c4ccccc4)n3)c3nc(-c4ccccc4)nc(-c4ccc5ccccc5c4-c4ccccc4)n3)cc2)c2c(C)cc(C)cc2C)c(C)c1]

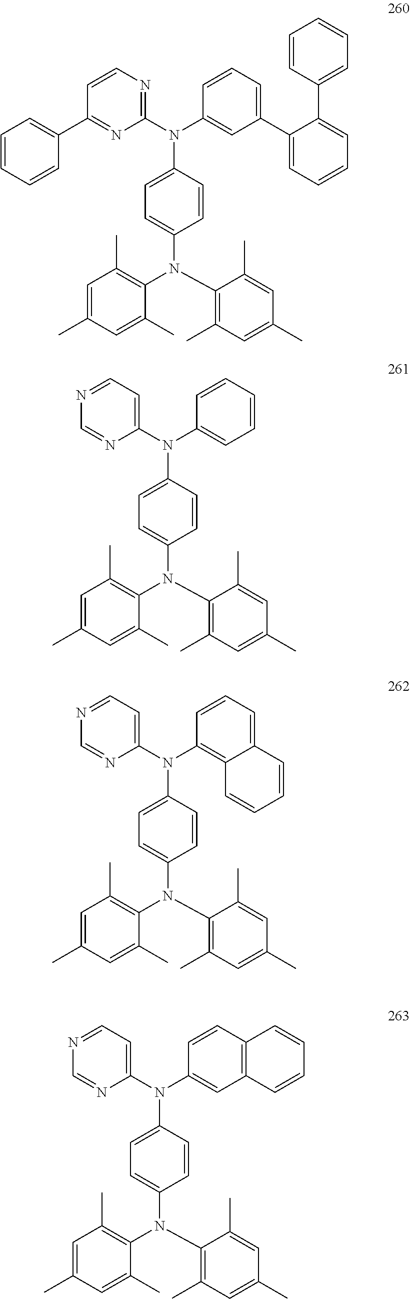

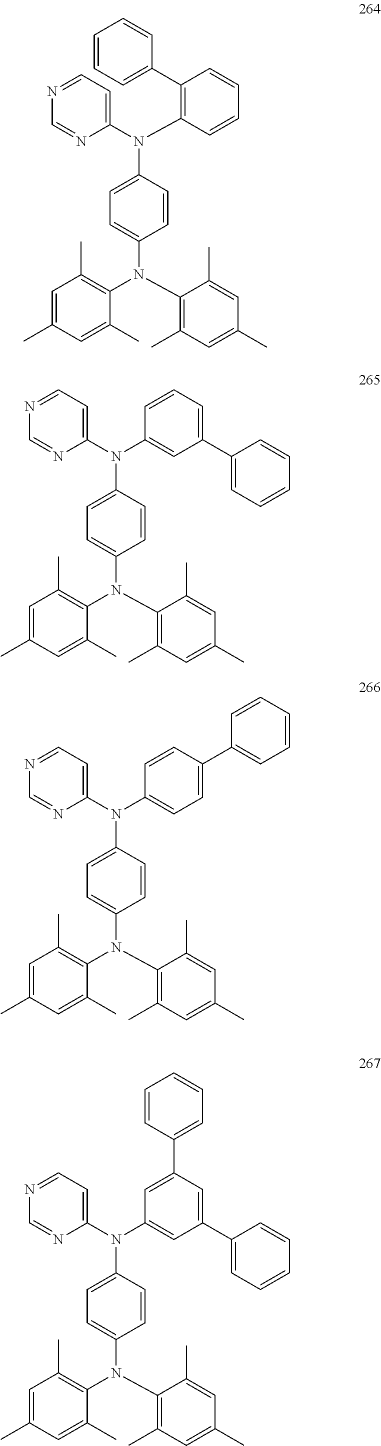

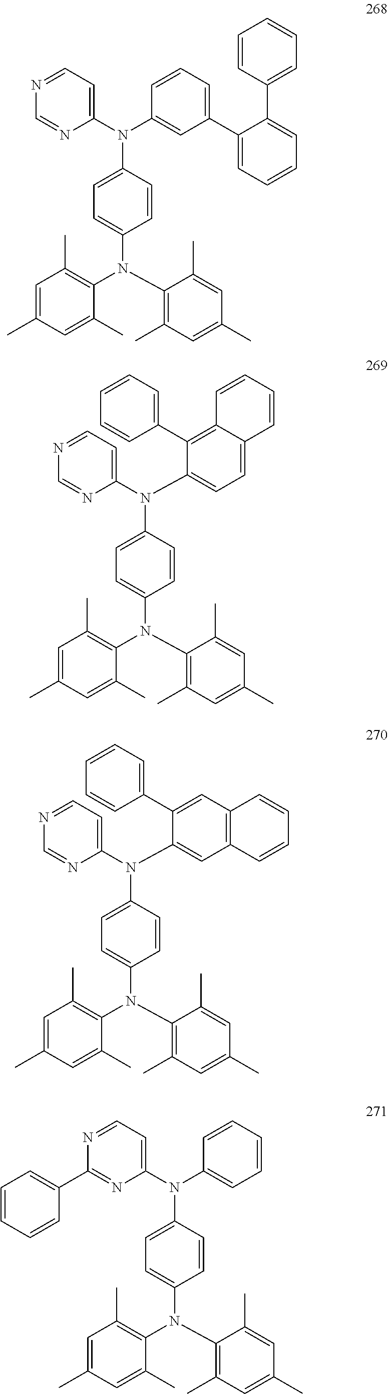

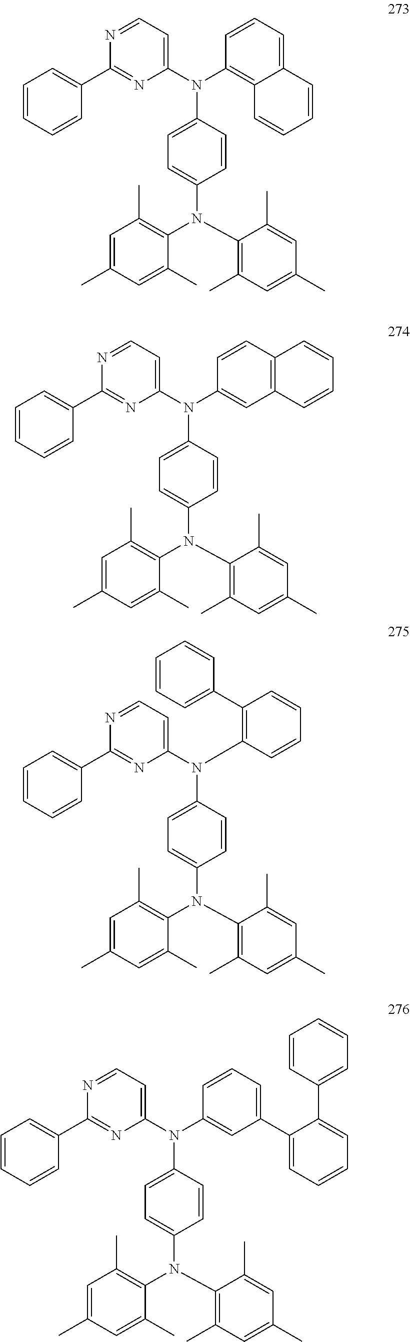

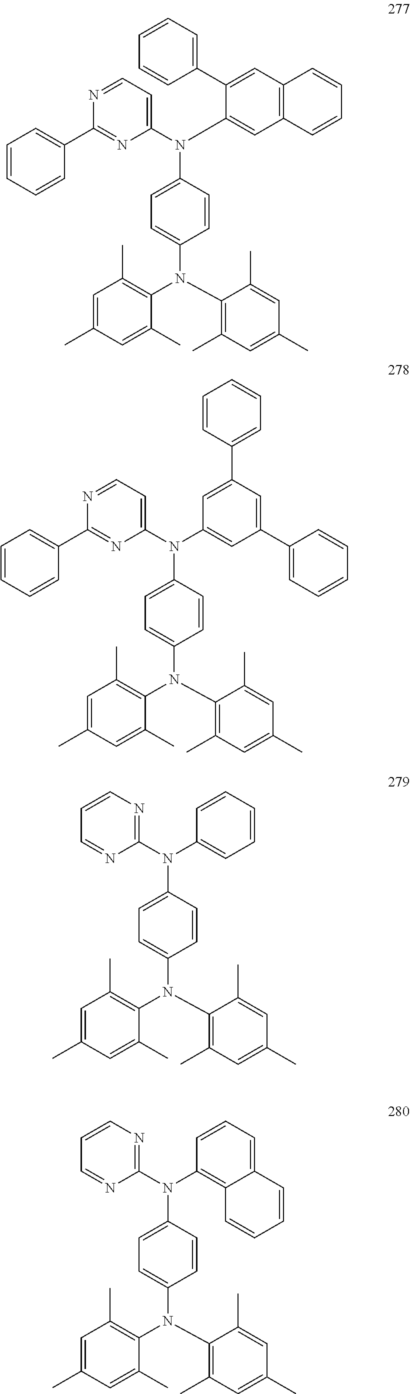

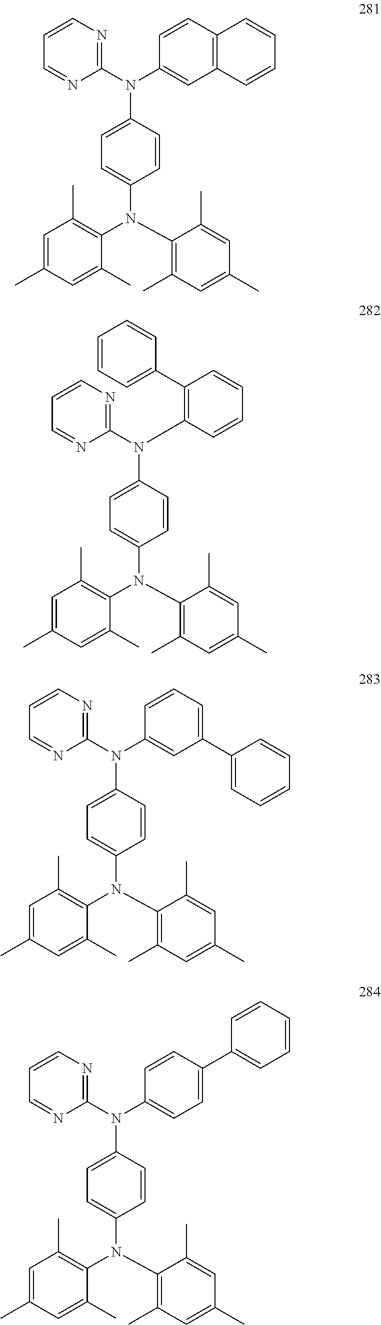

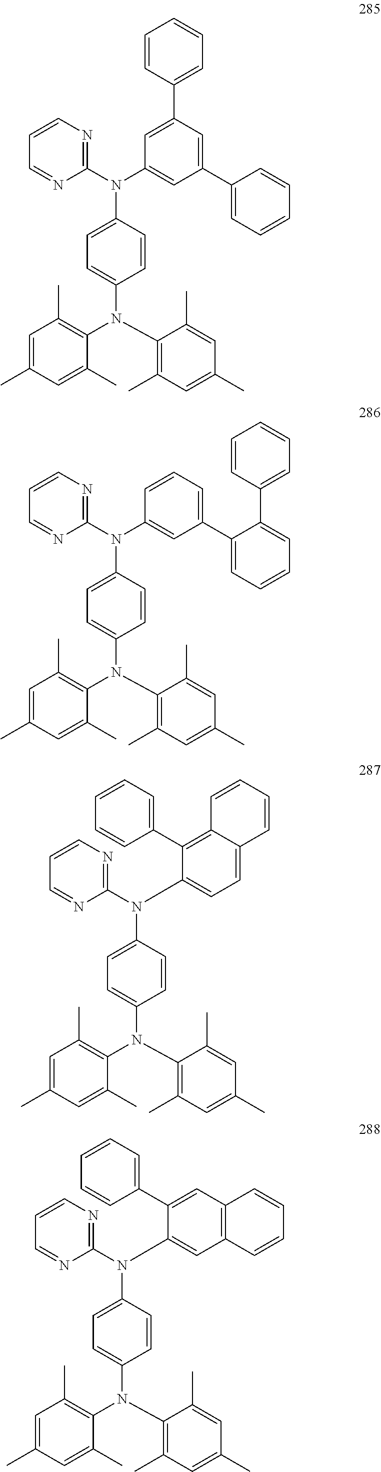

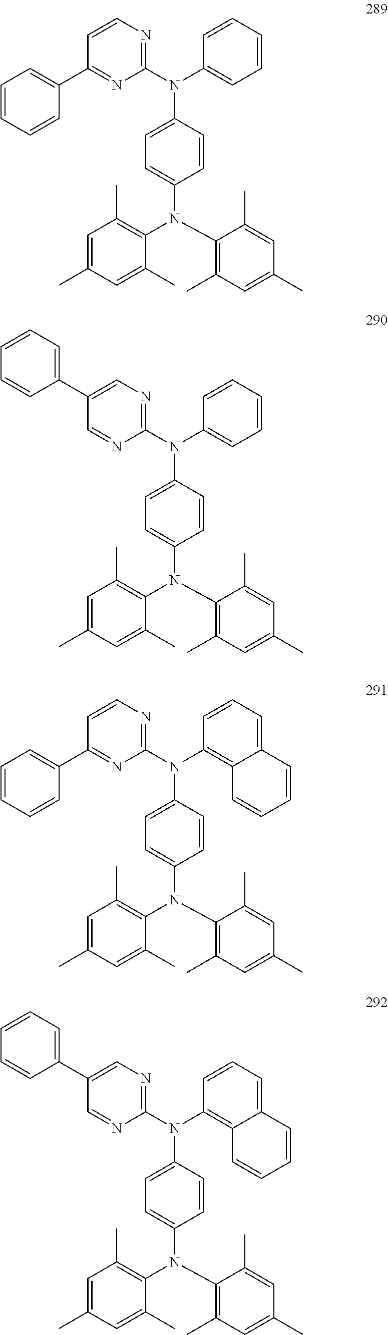 [SMILES: Cc1cc(C)c(N(c2ccc(N(c3ccccc3)c3nccc(-c4ccccc4)n3)cc2)c2c(C)cc(C)cc2C)c(C)c1 ; Cc1cc(C)c(N(c2ccc(N(c3ccccc3)c3ncc(-c4ccccc4)cn3)cc2)c2c(C)cc(C)cc2C)c(C)c1 ; Cc1cc(C)c(N(c2ccc(N(c3nccc(-c4ccccc4)n3)c3cccc4ccccc34)cc2)c2c(C)cc(C)cc2C)c(C)c1 ; Cc1cc(C)c(N(c2ccc(N(c3ncc(-c4ccccc4)cn3)c3cccc4ccccc34)cc2)c2c(C)cc(C)cc2C)c(C)c1]

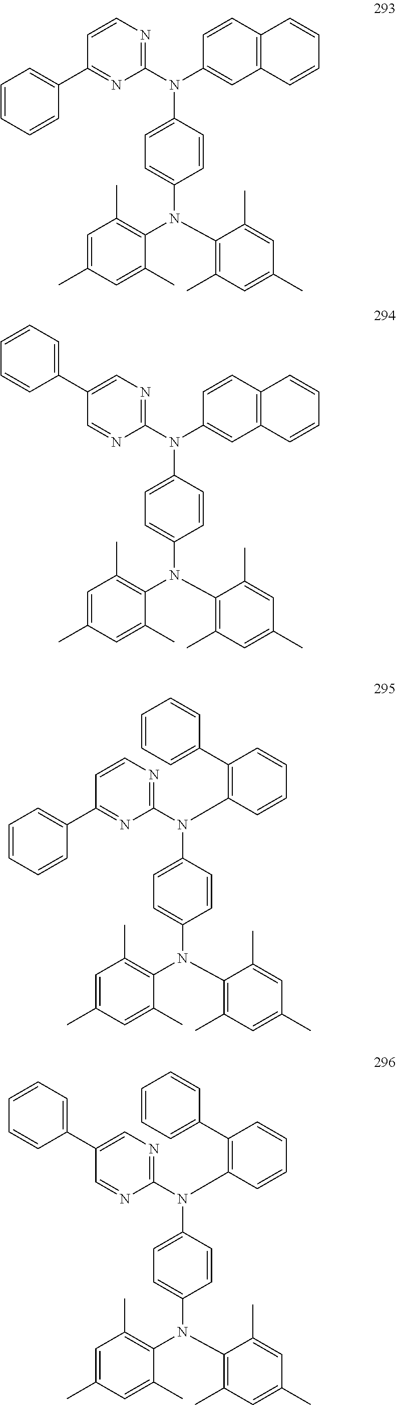 [SMILES: Cc1cc(C)c(N(c2ccc(N(c3ccc4ccccc4c3)c3nccc(-c4ccccc4)n3)cc2)c2c(C)cc(C)cc2C)c(C)c1 ; Cc1cc(C)c(N(c2ccc(N(c3ccc4ccccc4c3)c3ncc(-c4ccccc4)cn3)cc2)c2c(C)cc(C)cc2C)c(C)c1 ; Cc1cc(C)c(N(c2ccc(N(c3nccc(-c4ccccc4)n3)c3ccccc3-c3ccccc3)cc2)c2c(C)cc(C)cc2C)c(C)c1 ; Cc1cc(C)c(N(c2ccc(N(c3ncc(-c4ccccc4)cn3)c3ccccc3-c3ccccc3)cc2)c2c(C)cc(C)cc2C)c(C)c1]

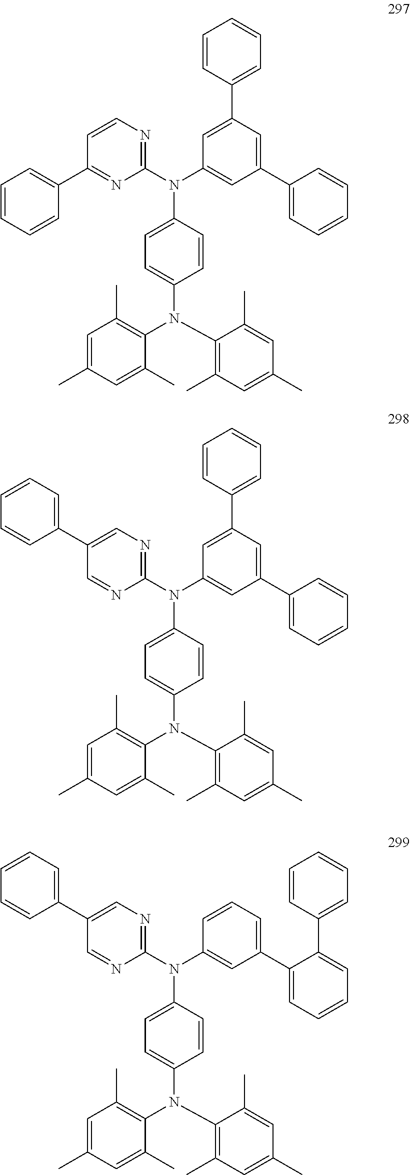

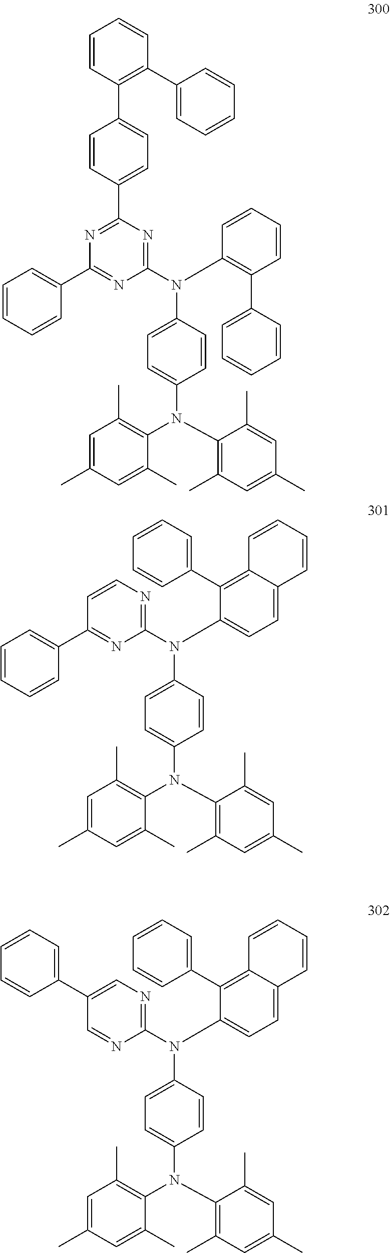

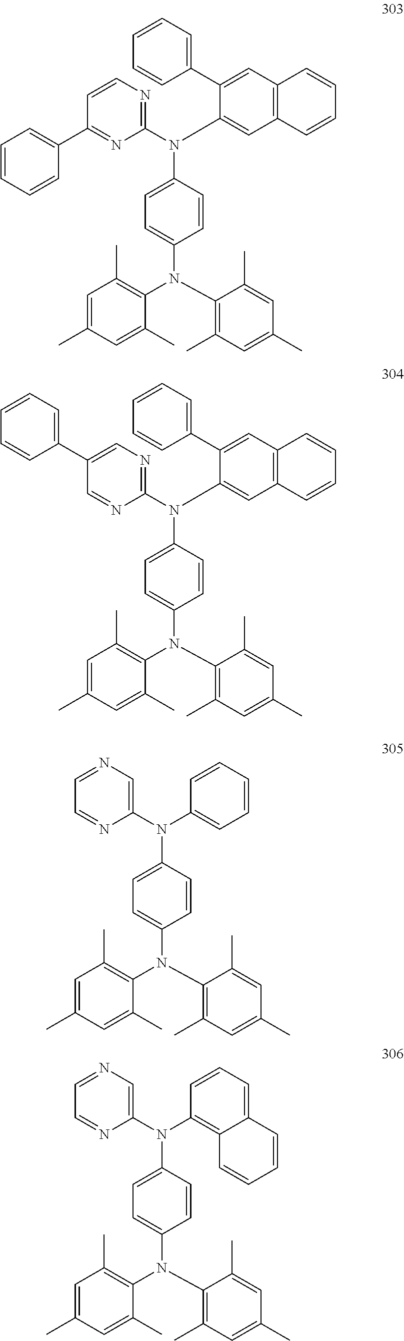

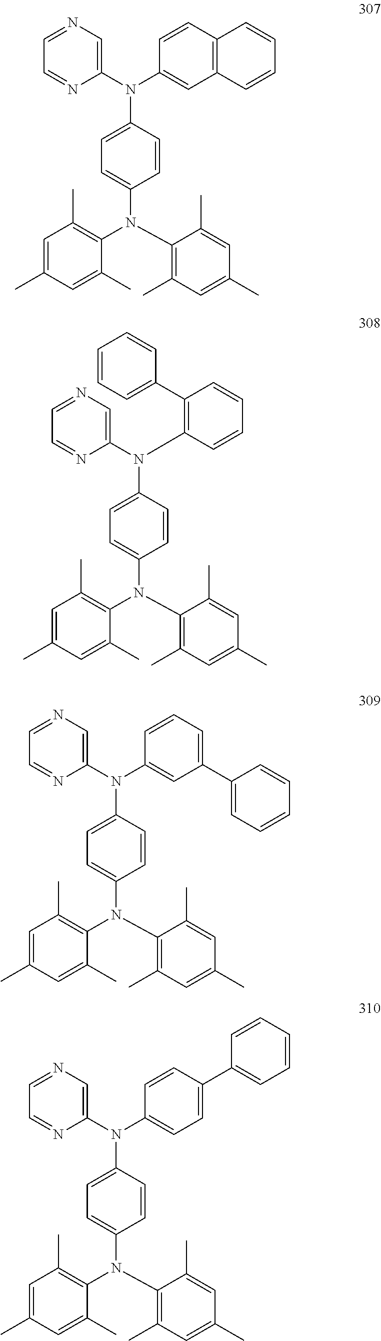

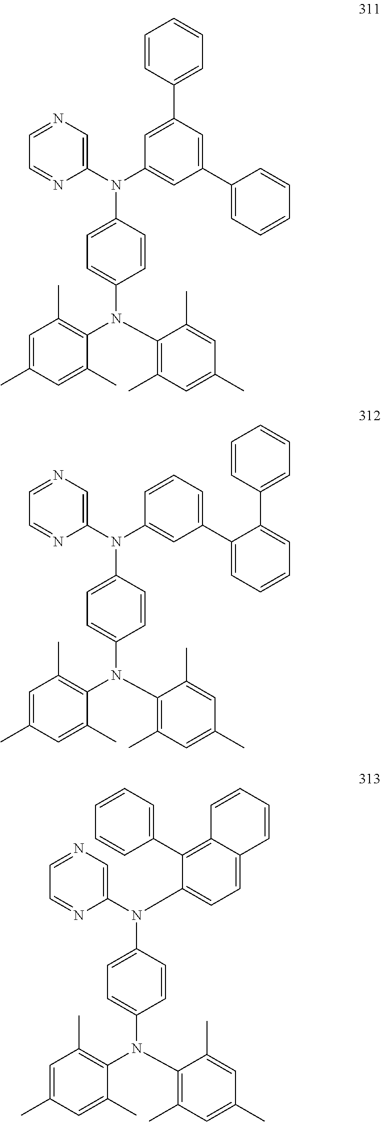

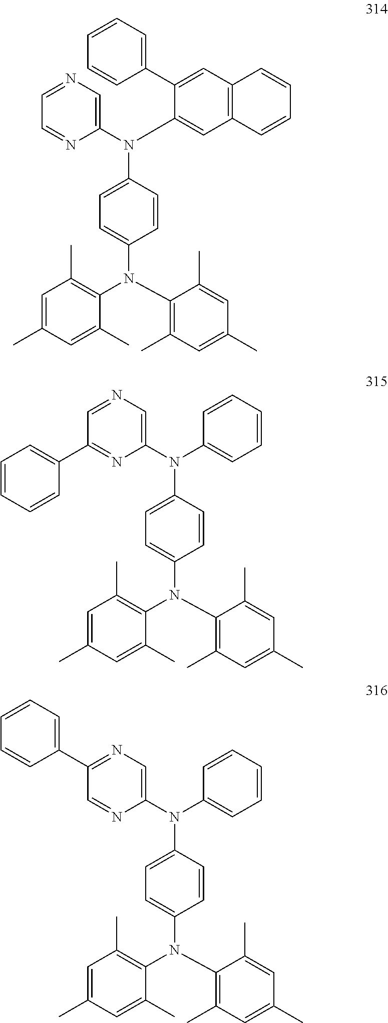 [SMILES: Cc1cc(C)c(N(c2ccc(N(c3cnccn3)c3cc4ccccc4cc3-c3ccccc3)cc2)c2c(C)cc(C)cc2C)c(C)c1 ; Cc1cc(C)c(N(c2ccc(N(c3ccccc3)c3cncc(-c4ccccc4)n3)cc2)c2c(C)cc(C)cc2C)c(C)c1 ; Cc1cc(C)c(N(c2ccc(N(c3ccccc3)c3cnc(-c4ccccc4)cn3)cc2)c2c(C)cc(C)cc2C)c(C)c1]

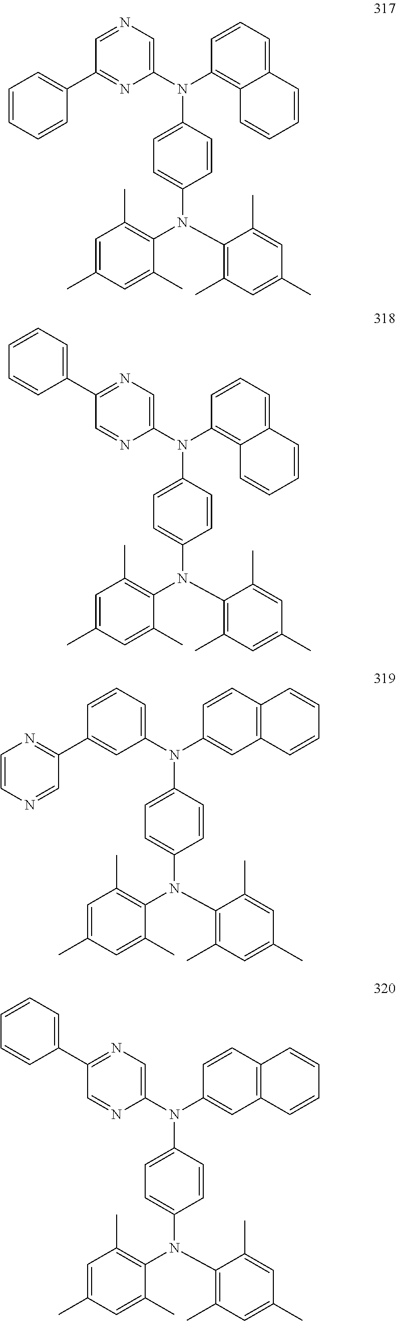

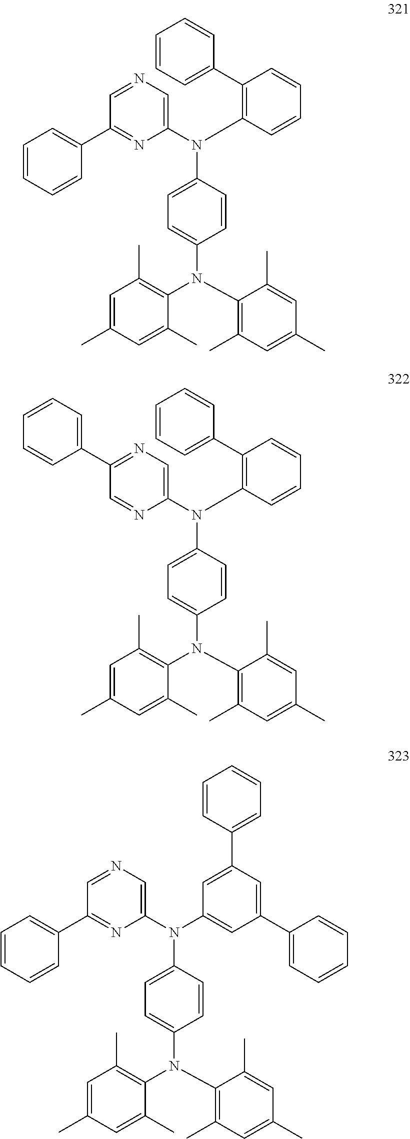

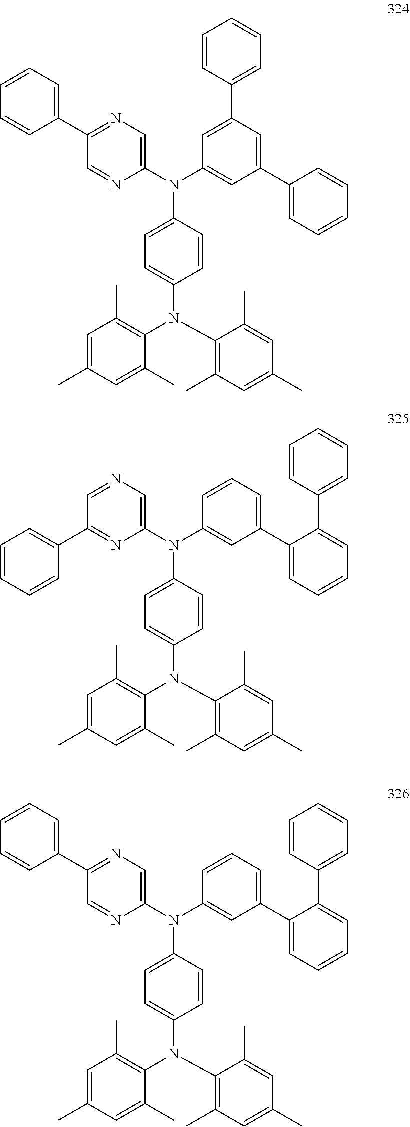

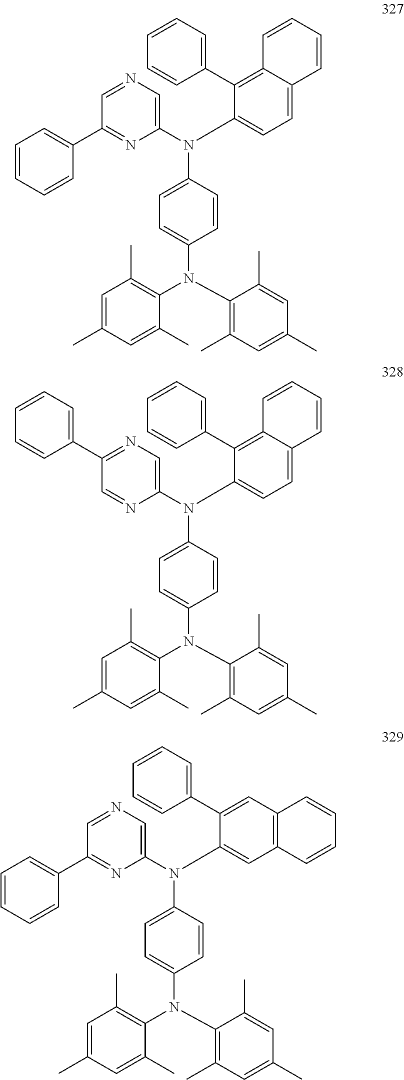

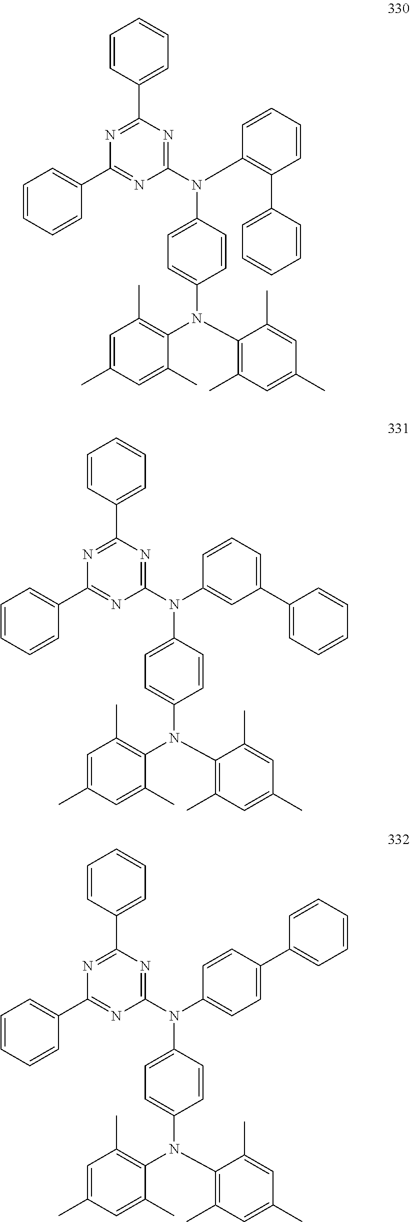

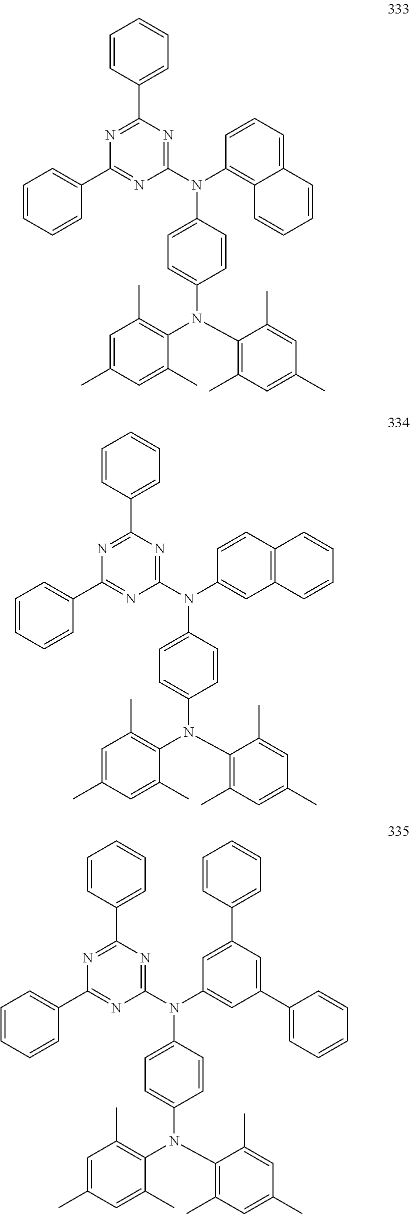

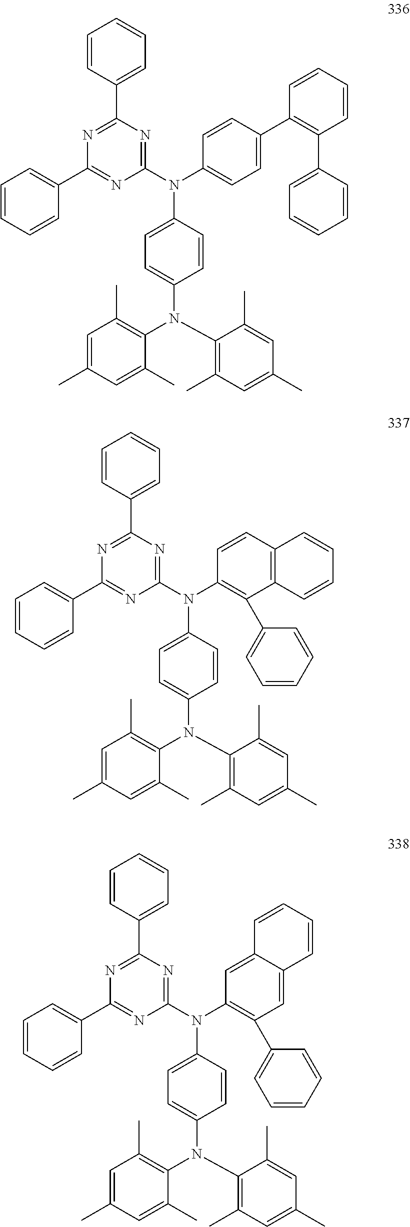 [SMILES: Cc1cc(C)c(N(c2ccc(N(c3ccc(-c4ccccc4-c4ccccc4)cc3)c3nc(-c4ccccc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4ccccc4)n3)c3cc4ccccc4cc3-c3ccccc3)cc2)c2c(C)cc(C)cc2C)c(C)c1]

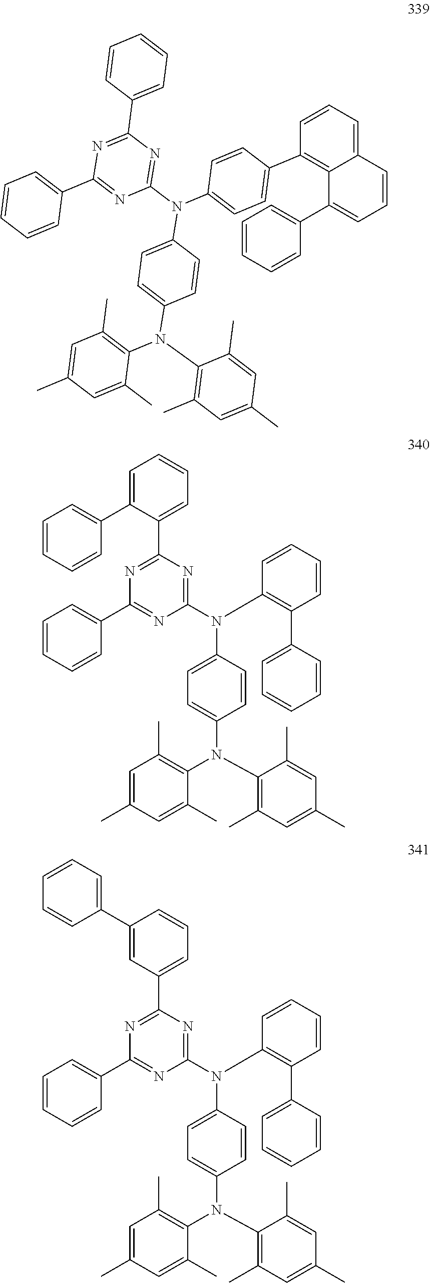

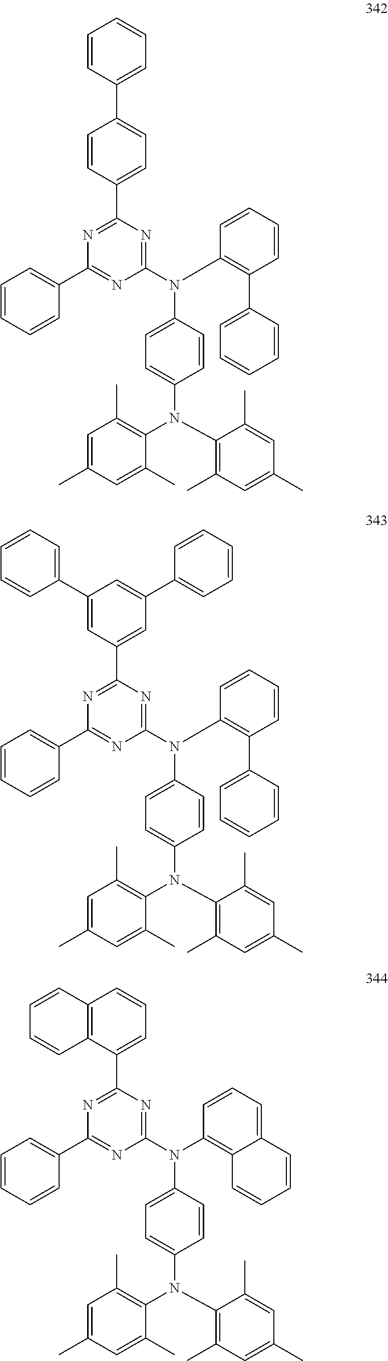

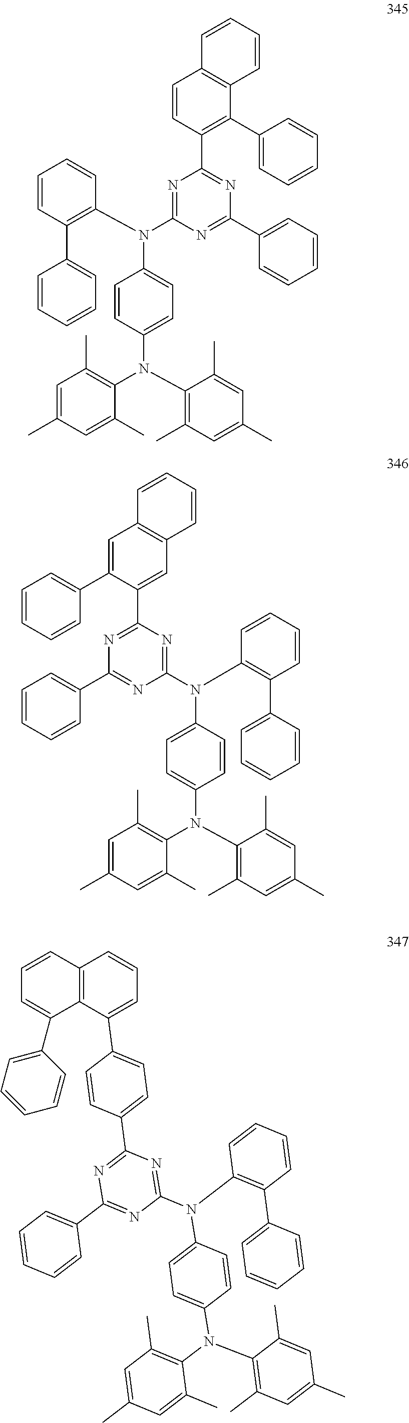

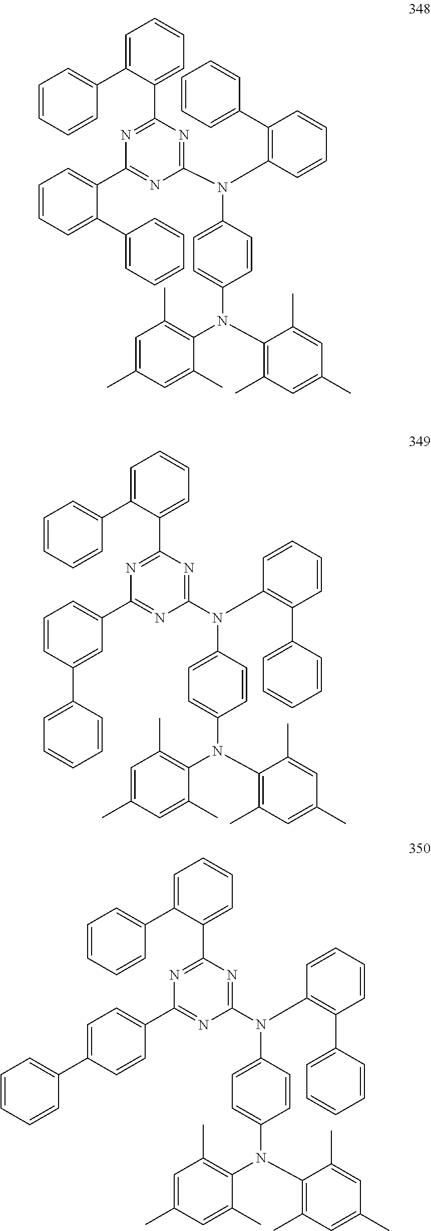

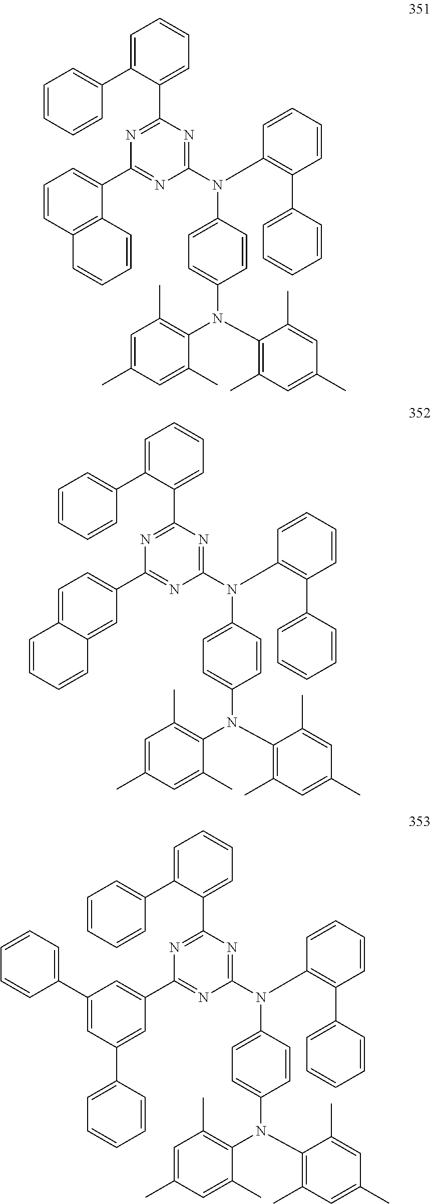

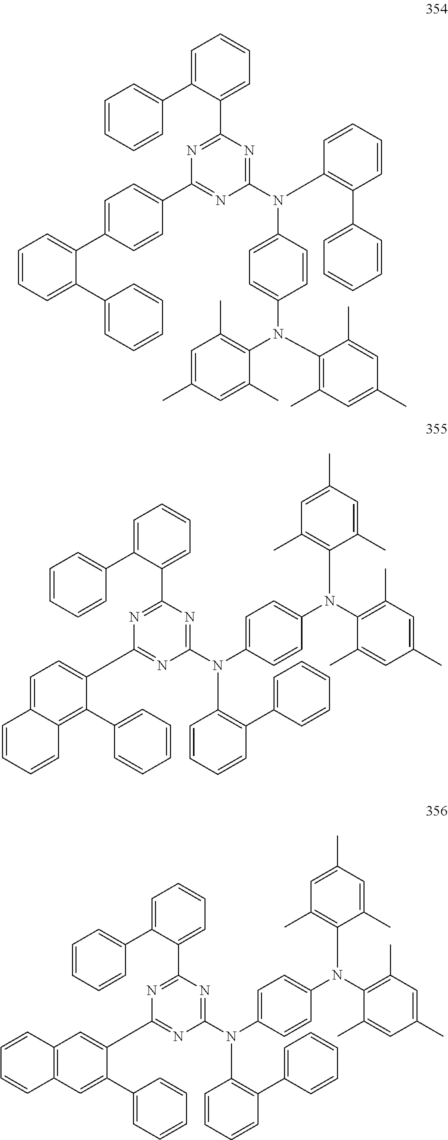 [SMILES: Cc1cc(C)c(N(c2ccc(N(c3nc(-c4ccc(-c5ccccc5-c5ccccc5)cc4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4ccccc4-c4ccccc4)nc(-c4ccc5ccccc5c4-c4ccccc4)n3)c3ccccc3-c3ccccc3)cc2)c2c(C)cc(C)cc2C)c(C)c1 ; Cc1cc(C)c(N(c2ccc(N(c3nc(-c4ccccc4-c4ccccc4)nc(-c4cc5ccccc5cc4-c4ccccc4)n3)c3ccccc3-c3ccccc3)cc2)c2c(C)cc(C)cc2C)c(C)c1]

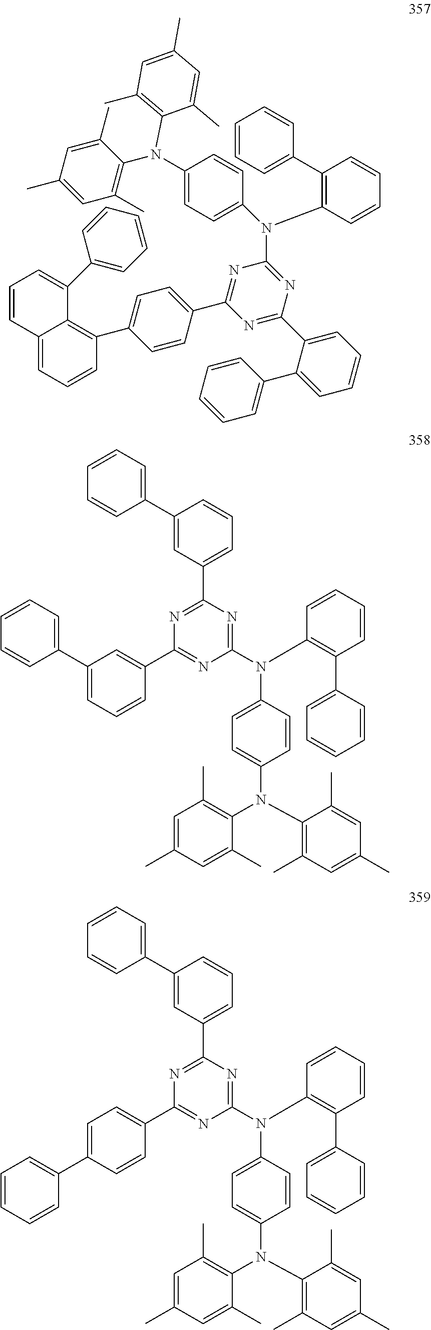 [SMILES: Cc1cc(C)c(N(c2ccc(N(c3nc(-c4ccc(-c5cccc6cccc(-c7ccccc7)c56)cc4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cccc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4ccc(-c5ccccc5)cc4)nc(-c4cccc(-c5ccccc5)c4)n3)c3ccccc3-c3ccccc3)cc2)c2c(C)cc(C)cc2C)c(C)c1]

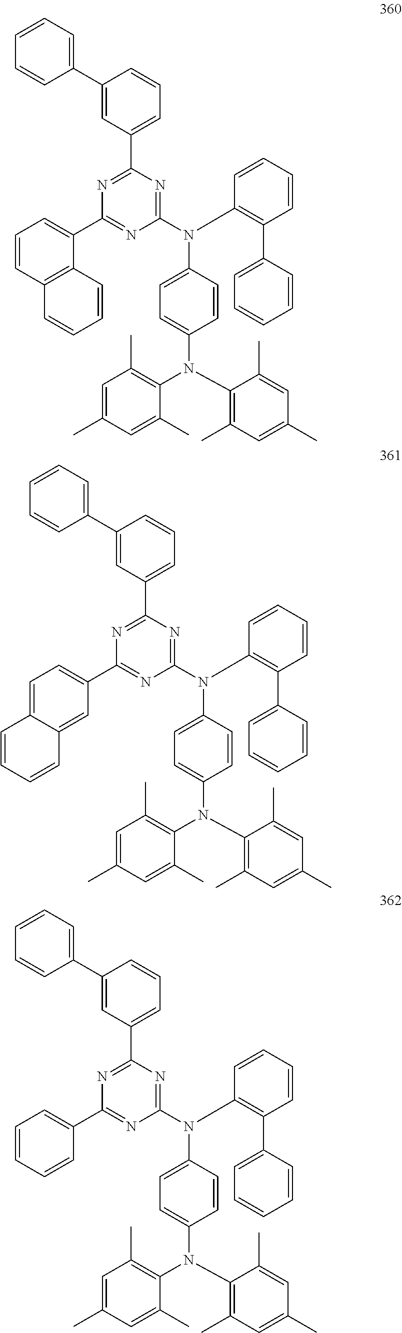

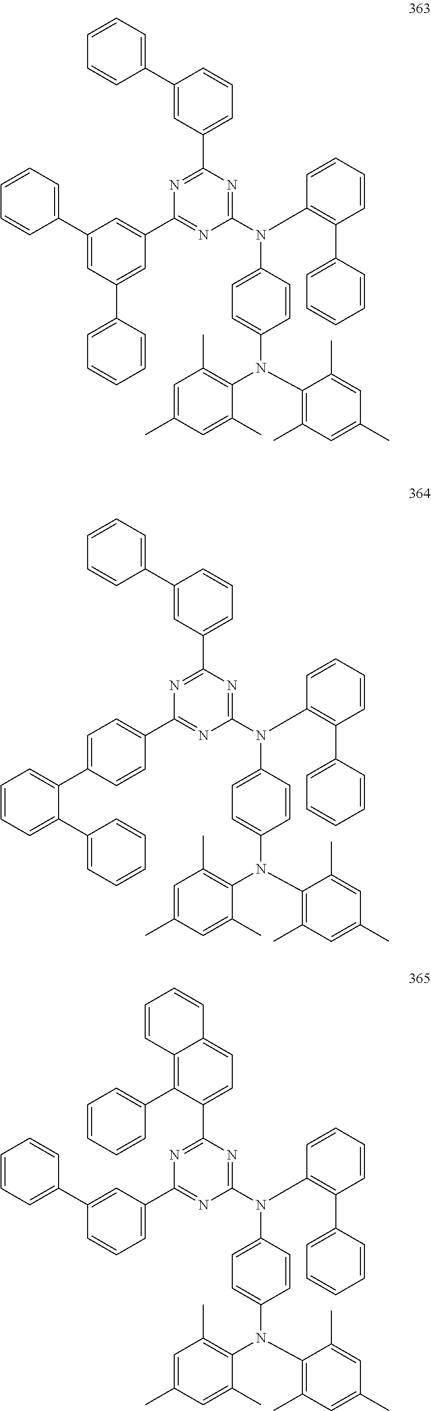 [SMILES: Cc1cc(C)c(N(c2ccc(N(c3nc(-c4cccc(-c5ccccc5)c4)nc(-c4cc(-c5ccccc5)cc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4ccc(-c5ccccc5-c5ccccc5)cc4)nc(-c4cccc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4ccc5ccccc5c4-c4ccccc4)n3)c3ccccc3-c3ccccc3)cc2)c2c(C)cc(C)cc2C)c(C)c1]

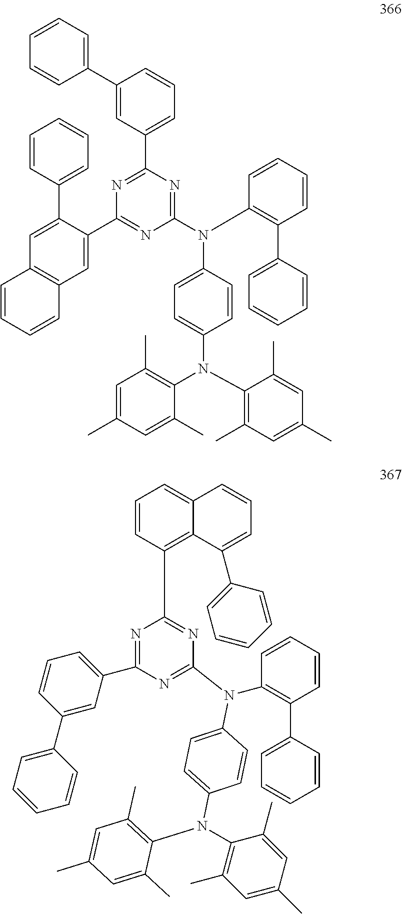

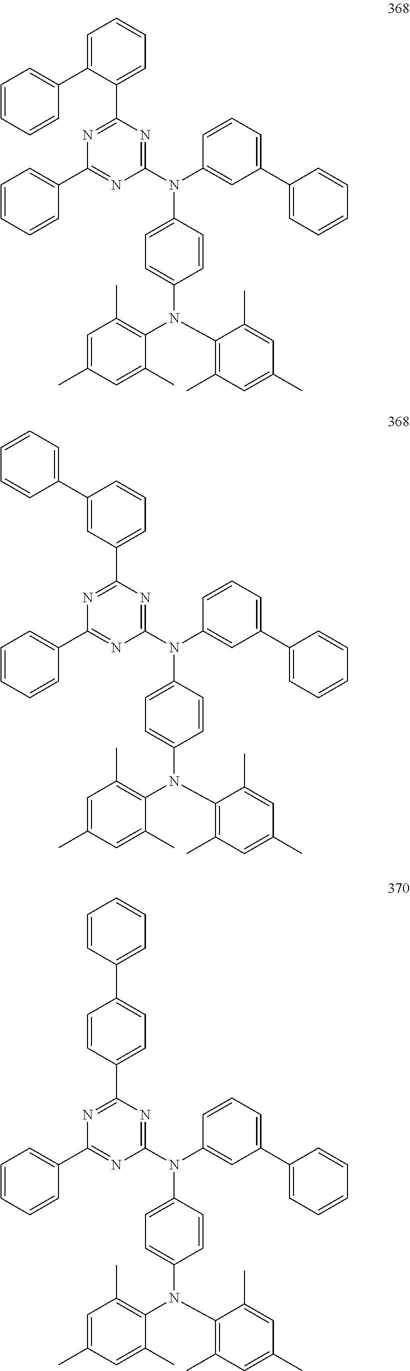 [SMILES: Cc1cc(C)c(N(c2ccc(N(c3cccc(-c4ccccc4)c3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4ccc(-c5ccccc5)cc4)n3)cc2)c2c(C)cc(C)cc2C)c(C)c1]

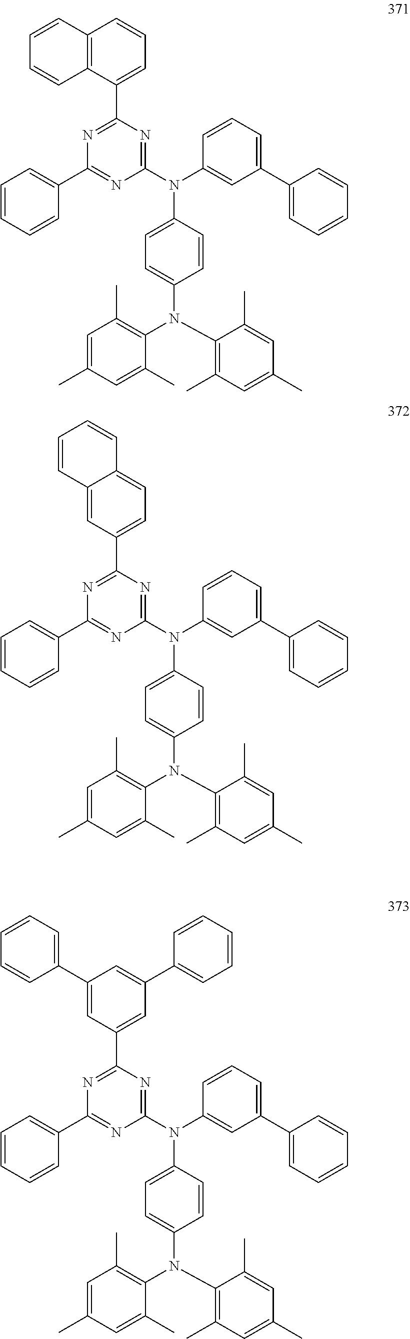

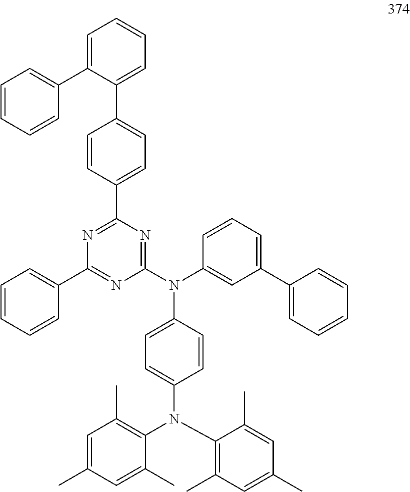 [SMILES: Cc1cc(C)c(N(c2ccc(N(c3cccc(-c4ccccc4)c3)c3nc(-c4ccccc4)nc(-c4ccc(-c5ccccc5-c5ccccc5)cc4)n3)cc2)c2c(C)cc(C)cc2C)c(C)c1]

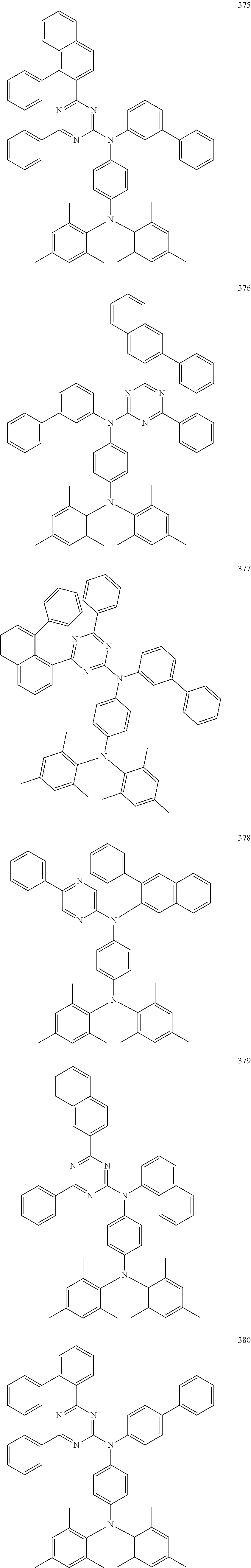

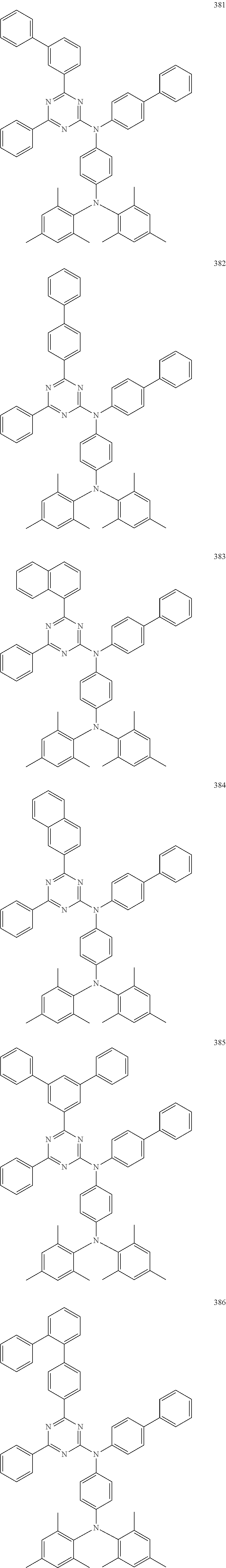

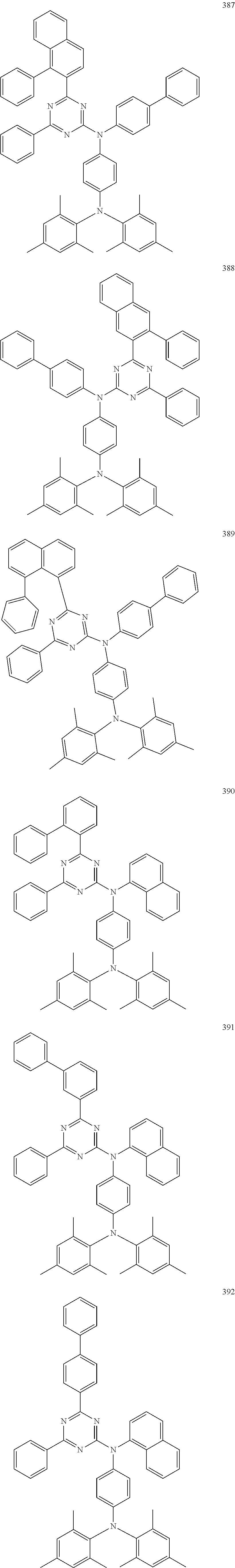

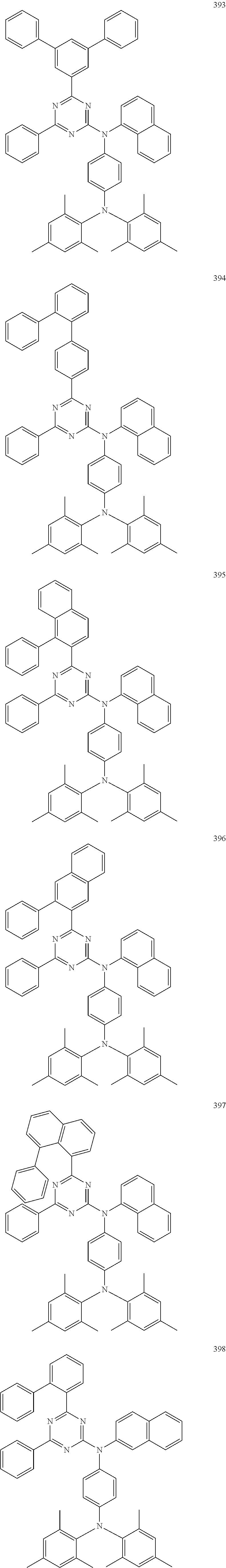

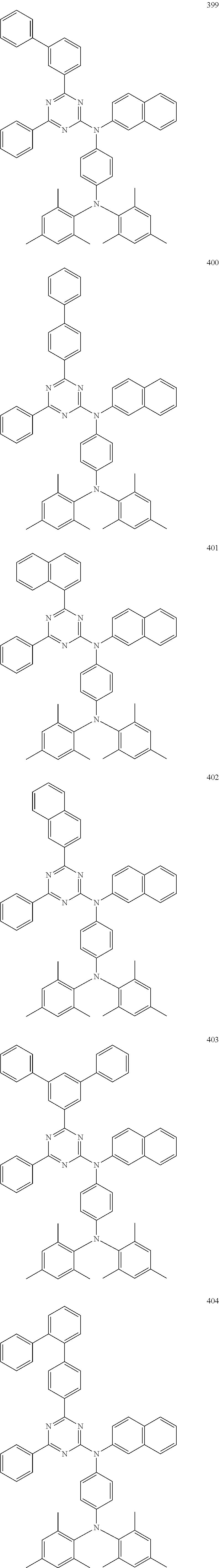

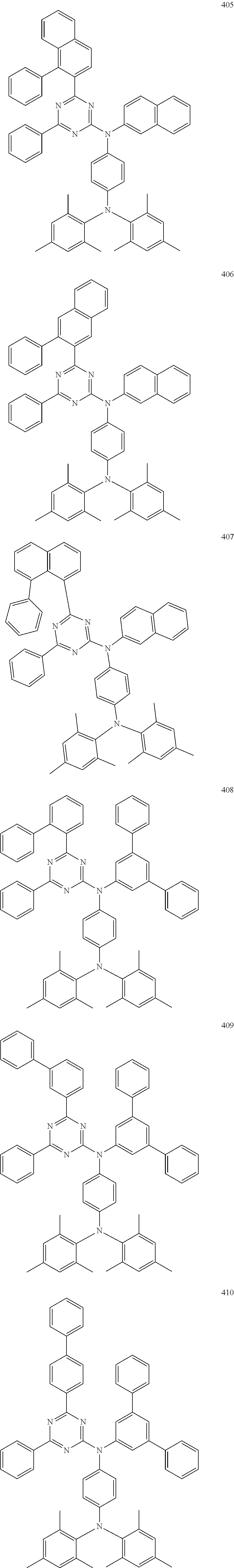

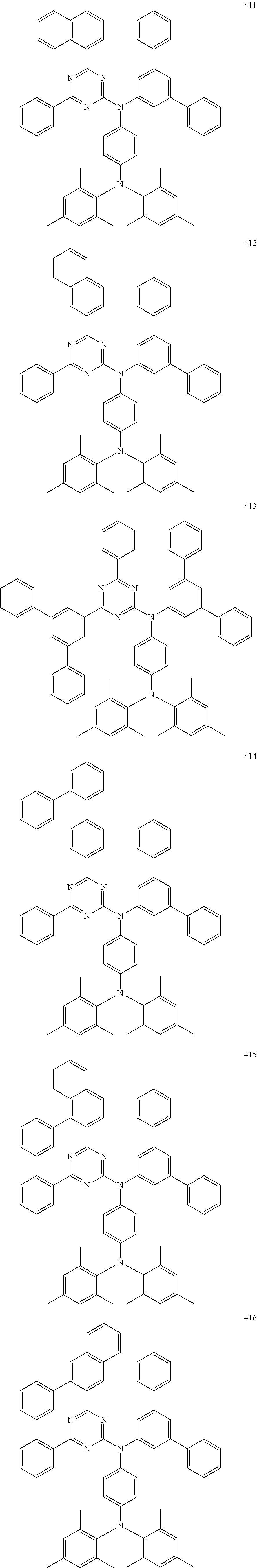

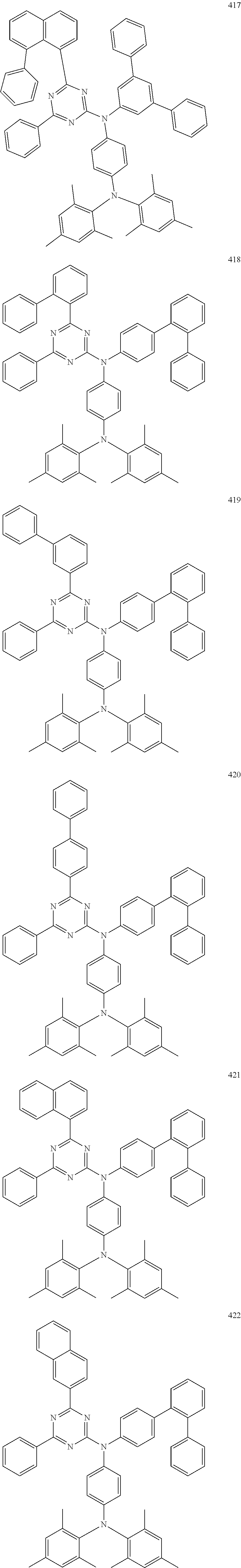

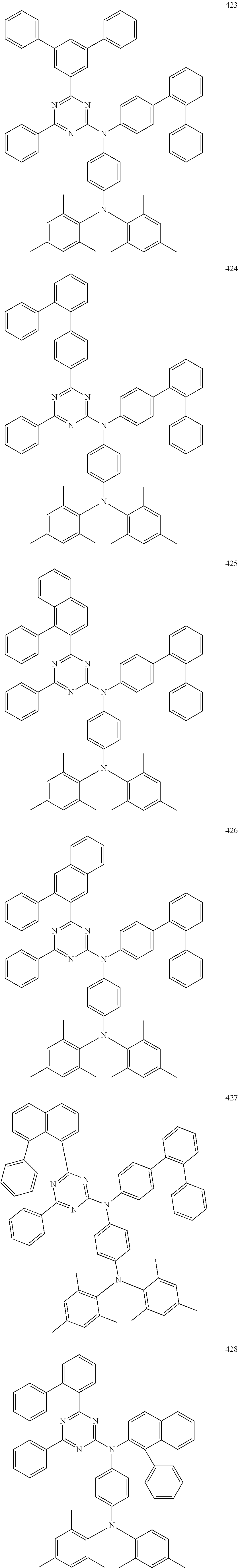

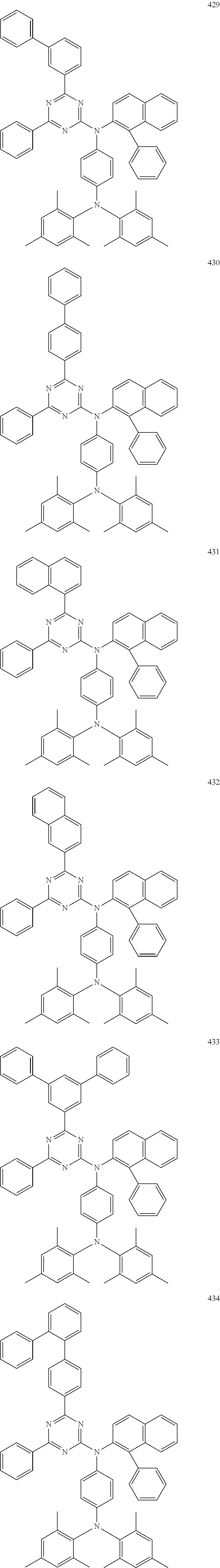

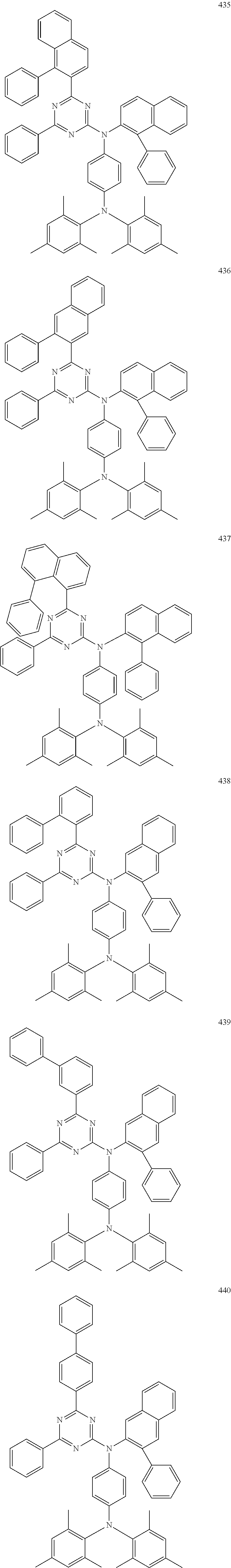

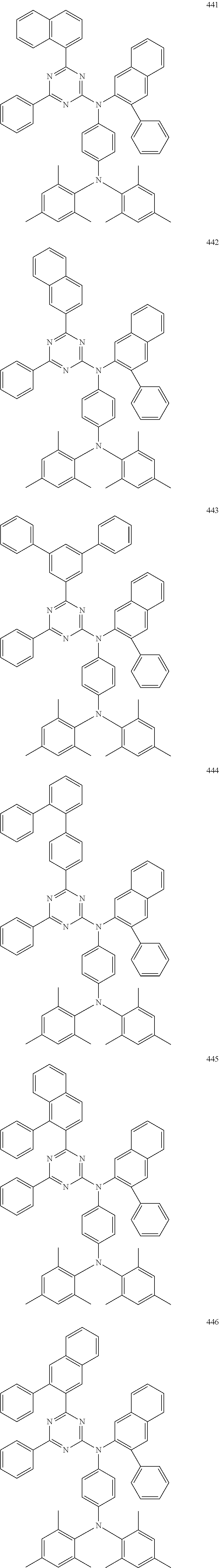

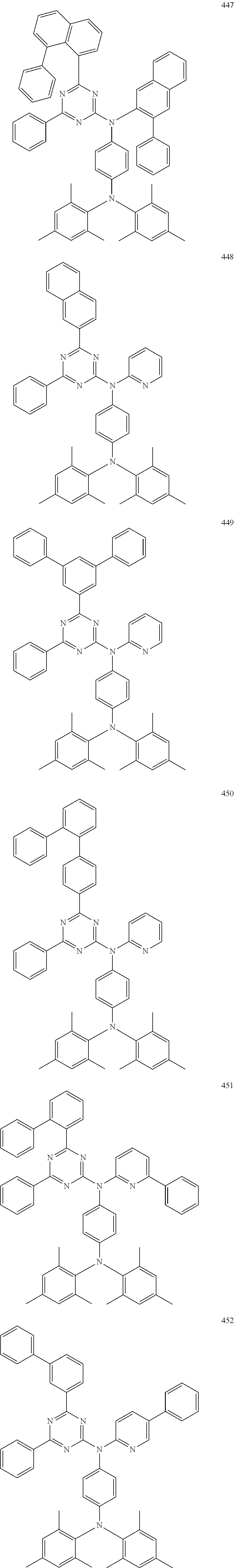

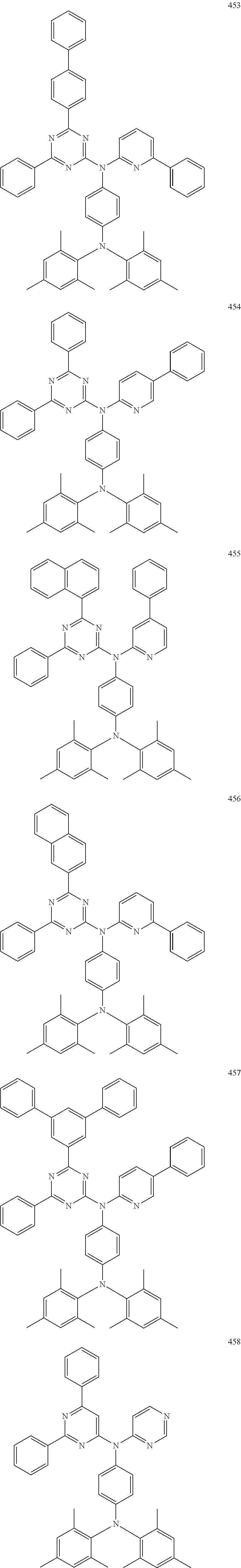

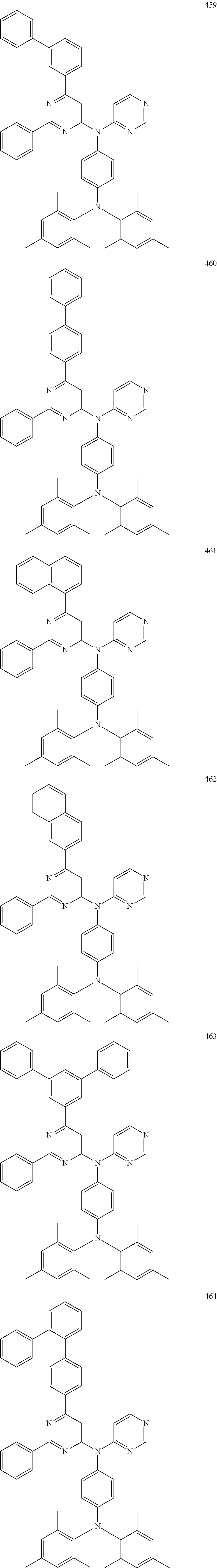

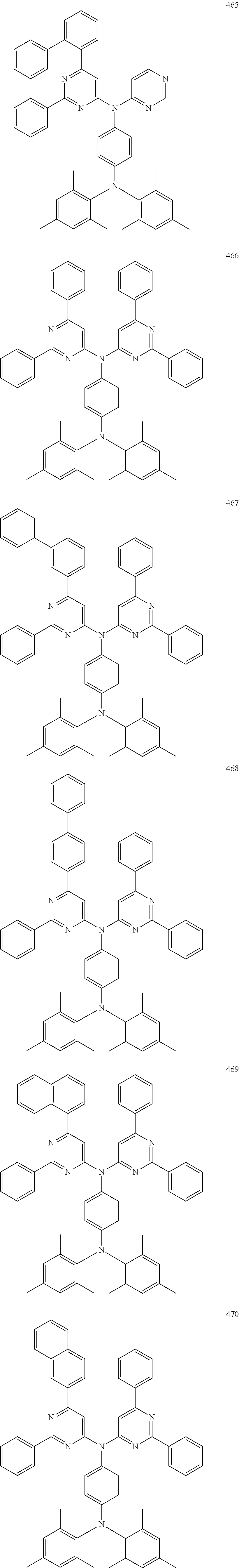

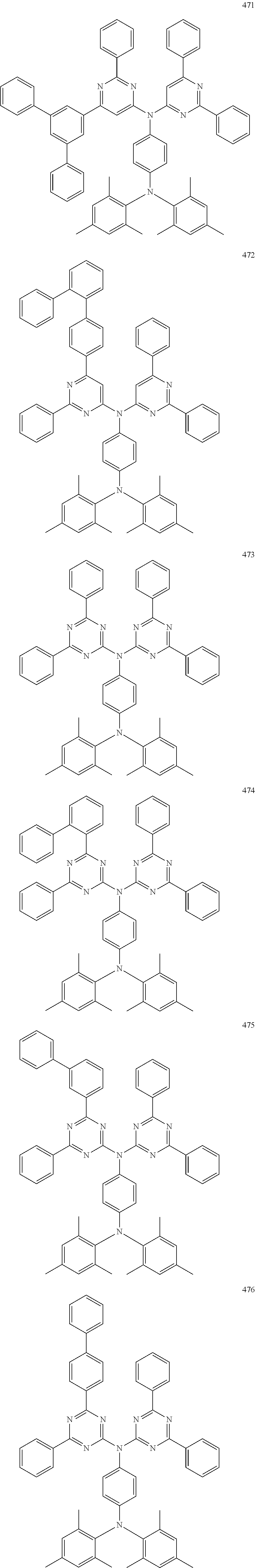

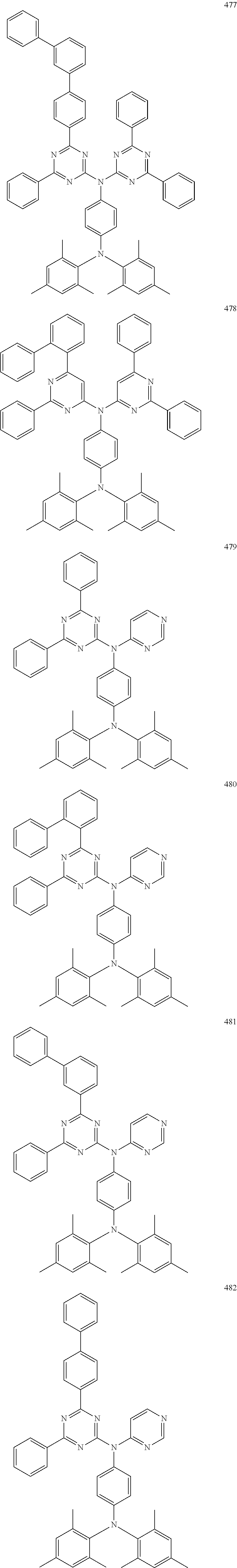

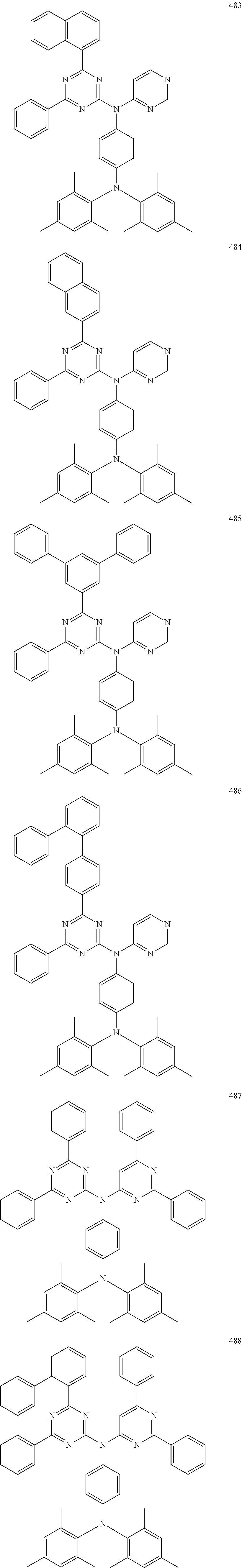

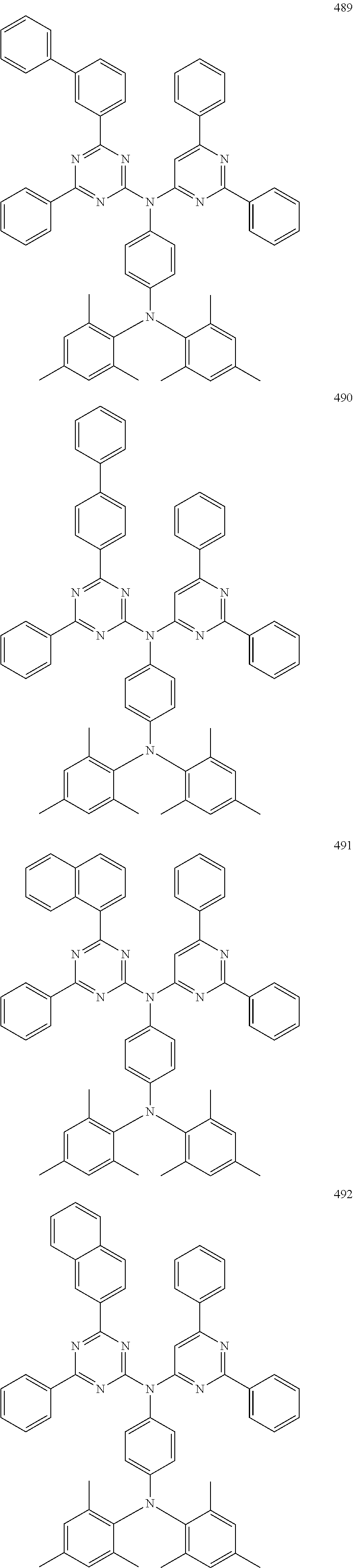

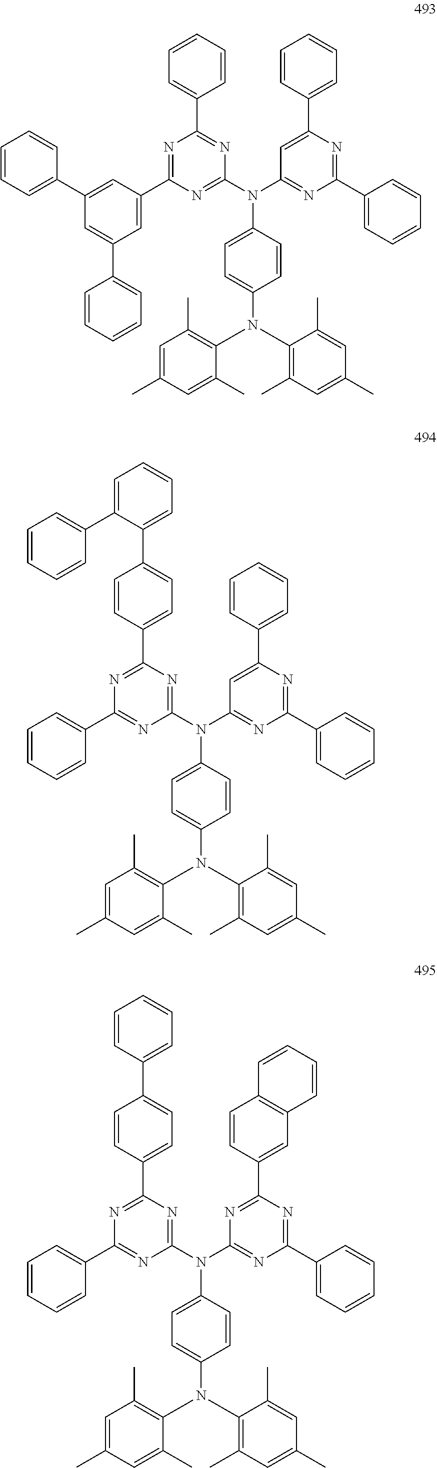 [SMILES: Cc1cc(C)c(N(c2ccc(N(c3cc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3cc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccc5ccccc5c4)n3)cc2)c2c(C)cc(C)cc2C)c(C)c1]

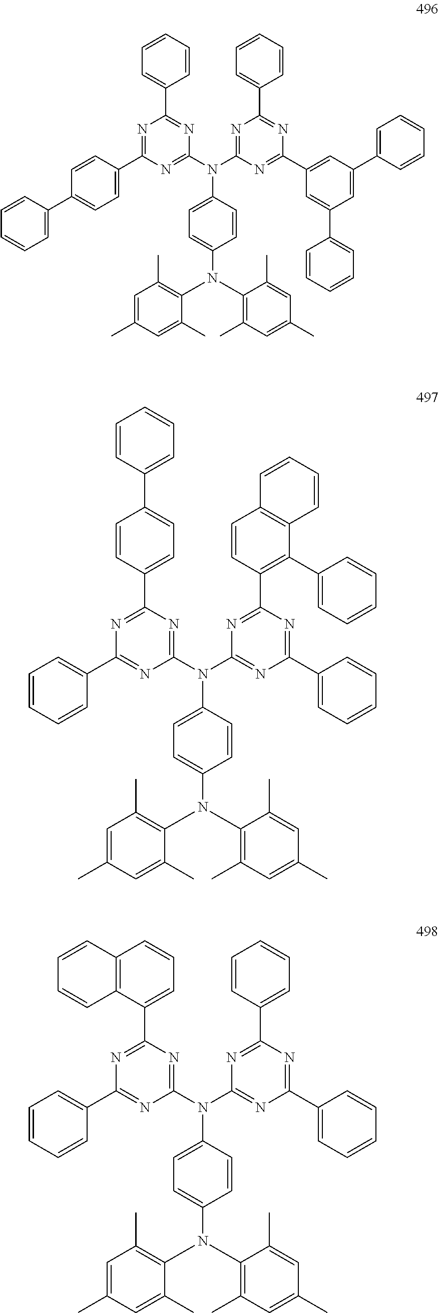 [SMILES: Cc1cc(C)c(N(c2ccc(N(c3nc(-c4ccccc4)nc(-c4ccc(-c5ccccc5)cc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cccc5ccccc45)n3)cc2)c2c(C)cc(C)cc2C)c(C)c1]

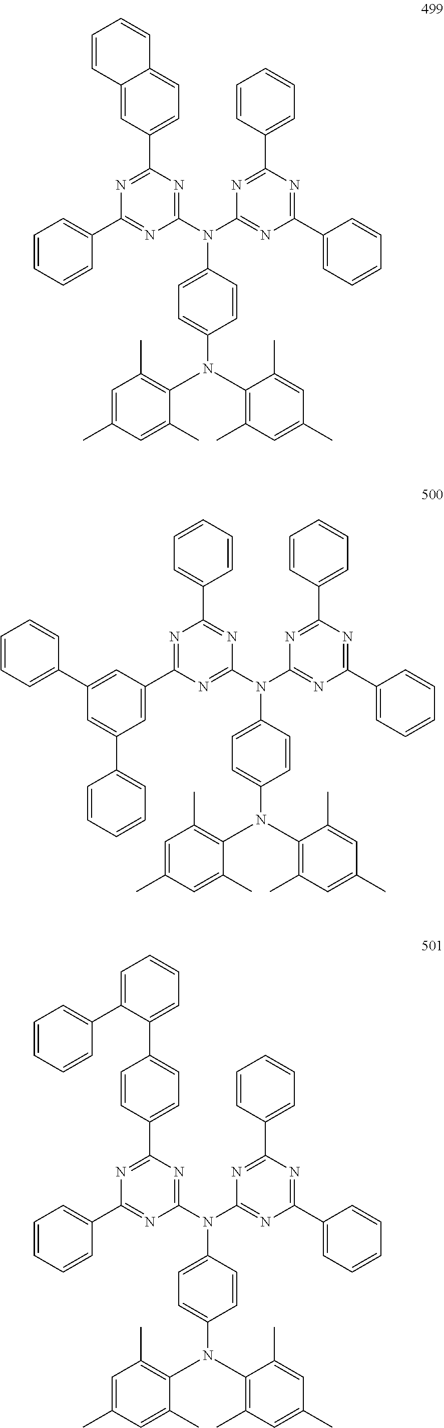 [SMILES: Cc1cc(C)c(N(c2ccc(N(c3nc(-c4ccccc4)nc(-c4ccccc4)n3)c3nc(-c4ccccc4)nc(-c4ccc5ccccc5c4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1]

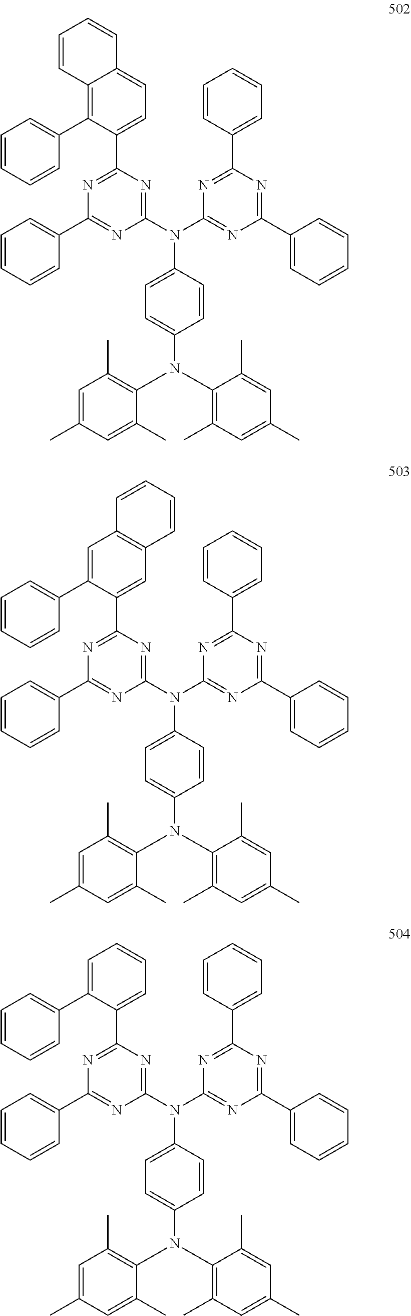

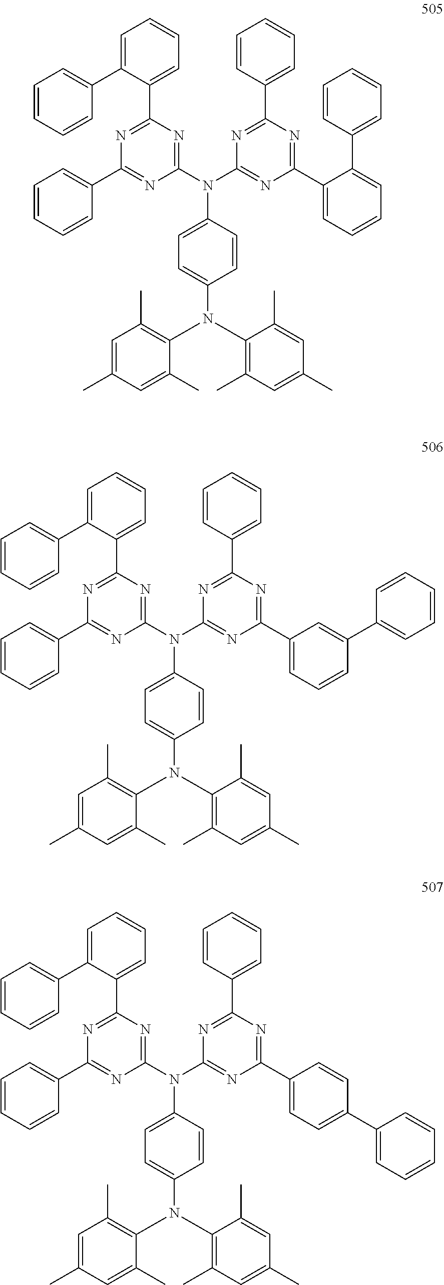 [SMILES: Cc1cc(C)c(N(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)cc(C)cc2C)c(C)c1]

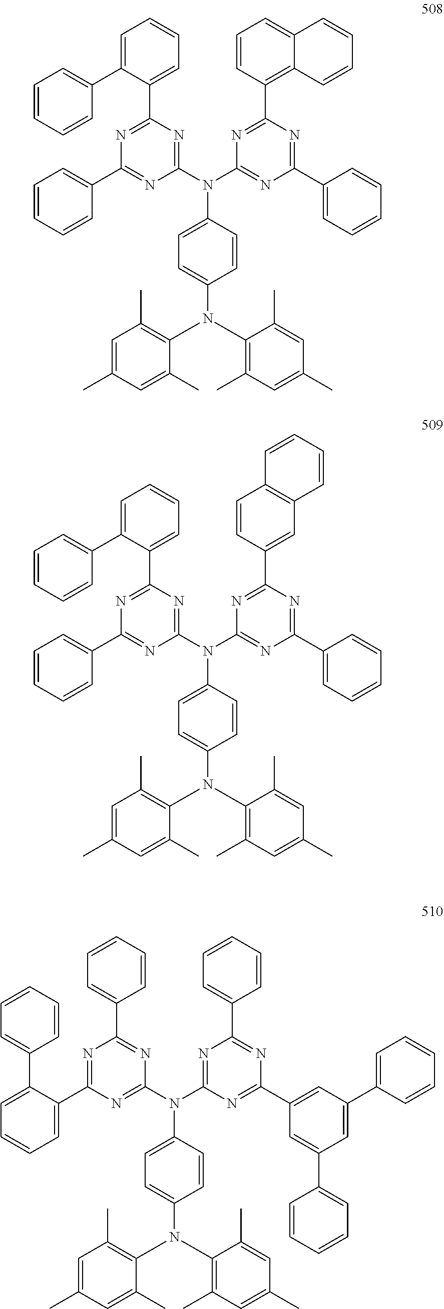 [SMILES: Cc1cc(C)c(N(c2ccc(N(c3nc(-c4ccccc4)nc(-c4ccccc4-c4ccccc4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccccc4-c4ccccc4)n3)cc2)c2c(C)cc(C)cc2C)c(C)c1]

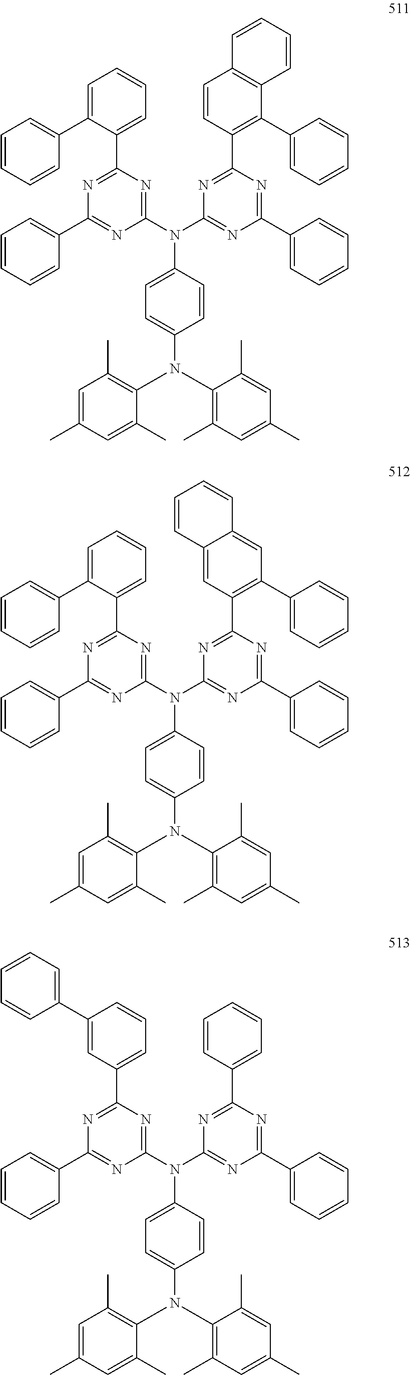

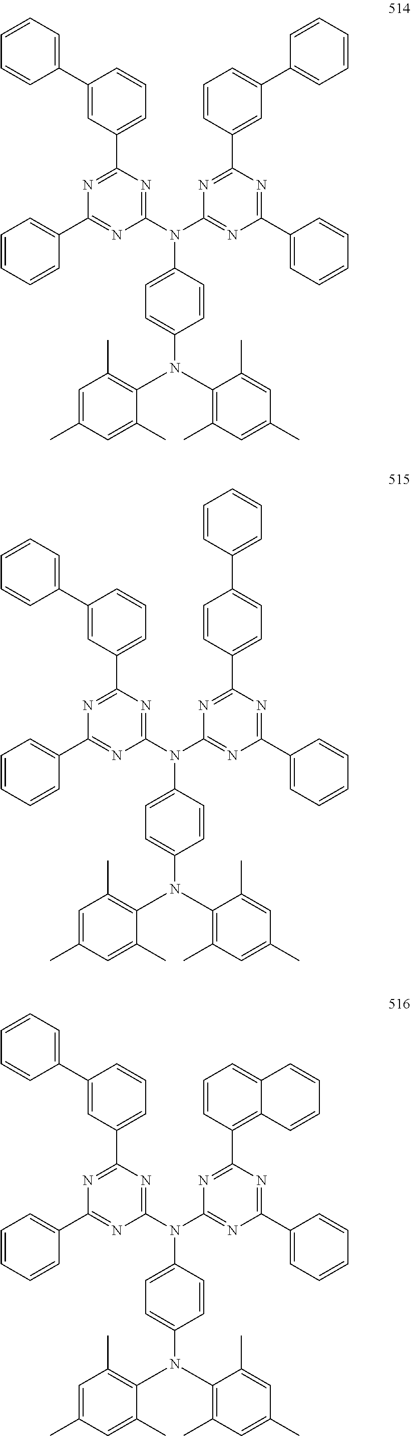 [SMILES: Cc1cc(C)c(N(c2ccc(N(c3nc(-c4ccccc4)nc(-c4cccc(-c5ccccc5)c4)n3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4cccc5ccccc45)n3)cc2)c2c(C)cc(C)cc2C)c(C)c1]

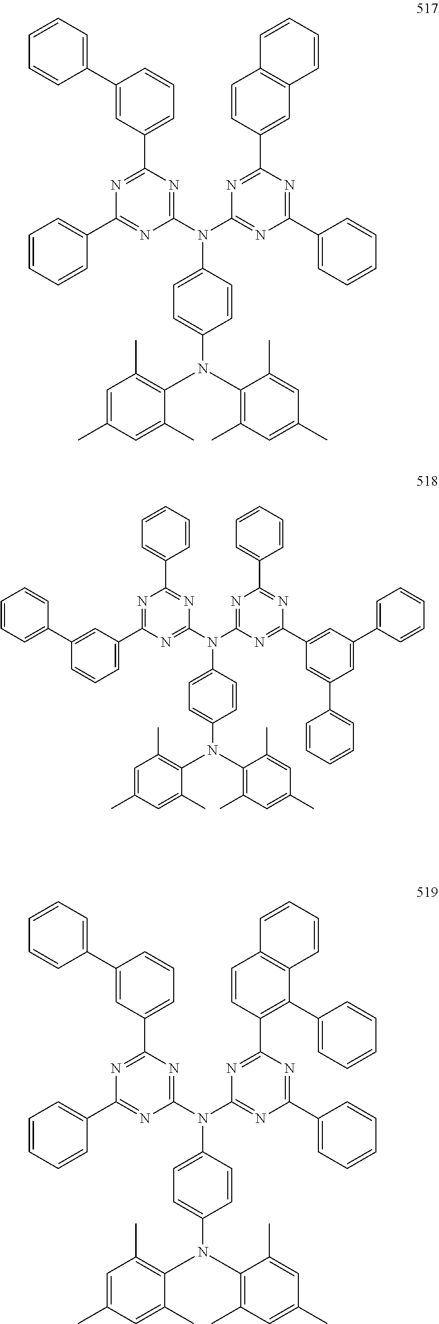 [SMILES: Cc1cc(C)c(N(c2ccc(N(c3nc(-c4ccccc4)nc(-c4cccc(-c5ccccc5)c4)n3)c3nc(-c4ccccc4)nc(-c4ccc5ccccc5c4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4ccc5ccccc5c4-c4ccccc4)n3)cc2)c2c(C)cc(C)cc2C)c(C)c1]

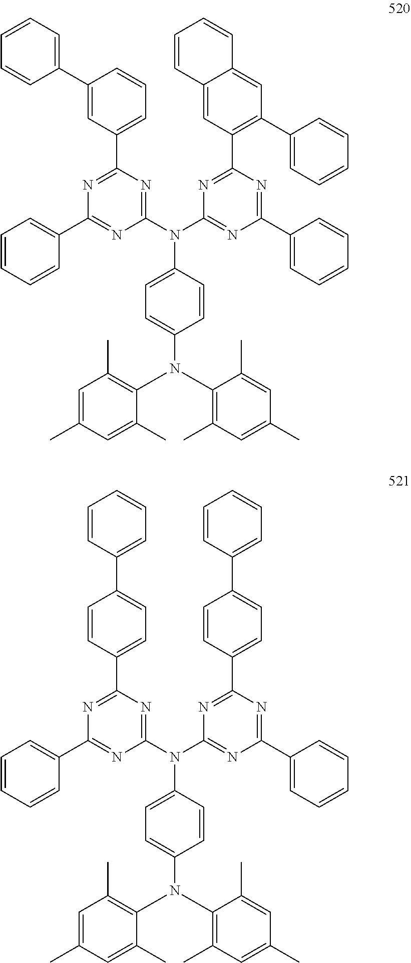 [SMILES: Cc1cc(C)c(N(c2ccc(N(c3nc(-c4ccccc4)nc(-c4cccc(-c5ccccc5)c4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccc(-c5ccccc5)cc4)n3)cc2)c2c(C)cc(C)cc2C)c(C)c1]

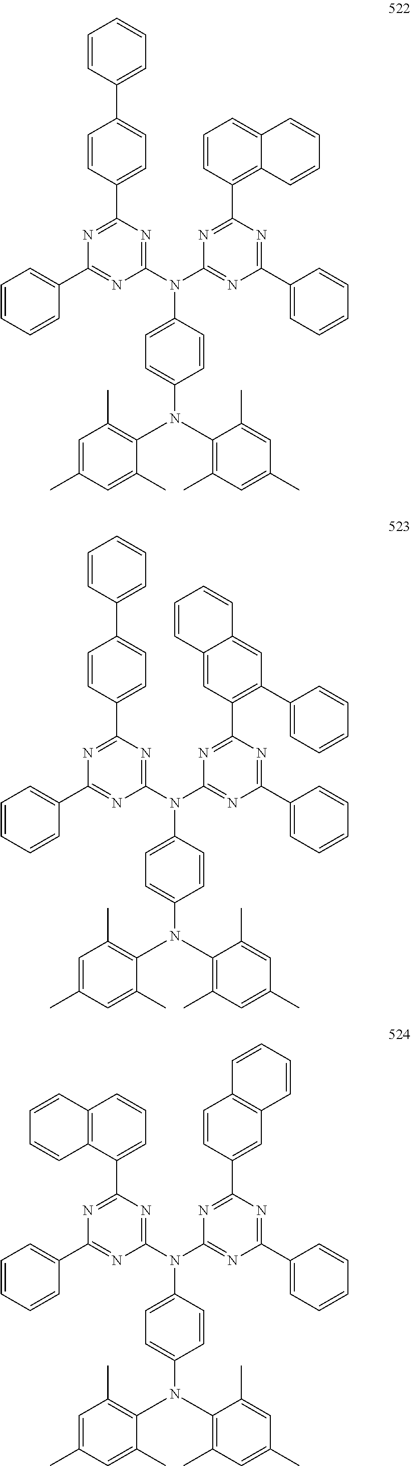

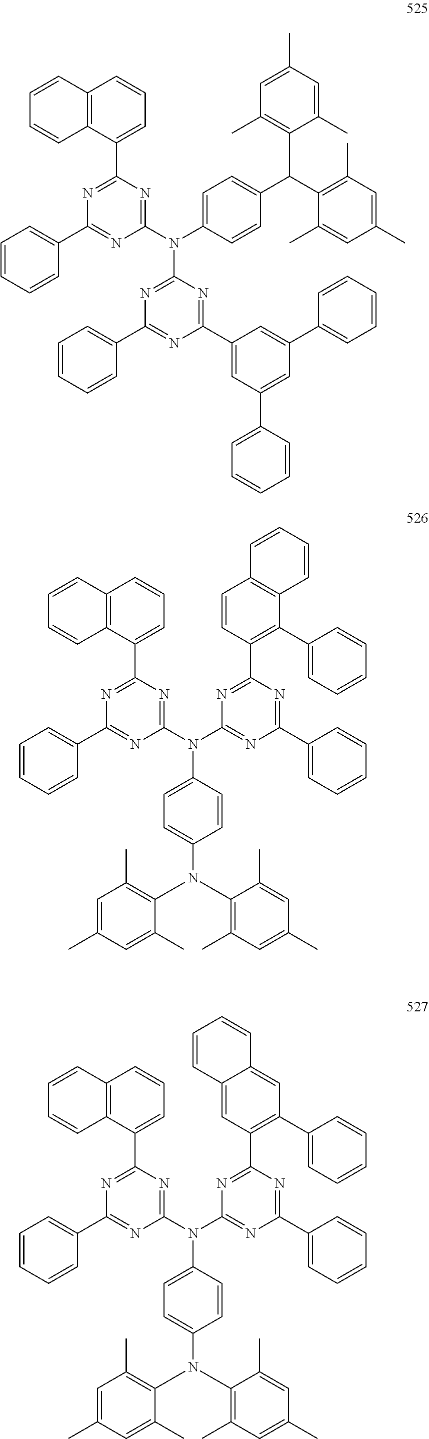

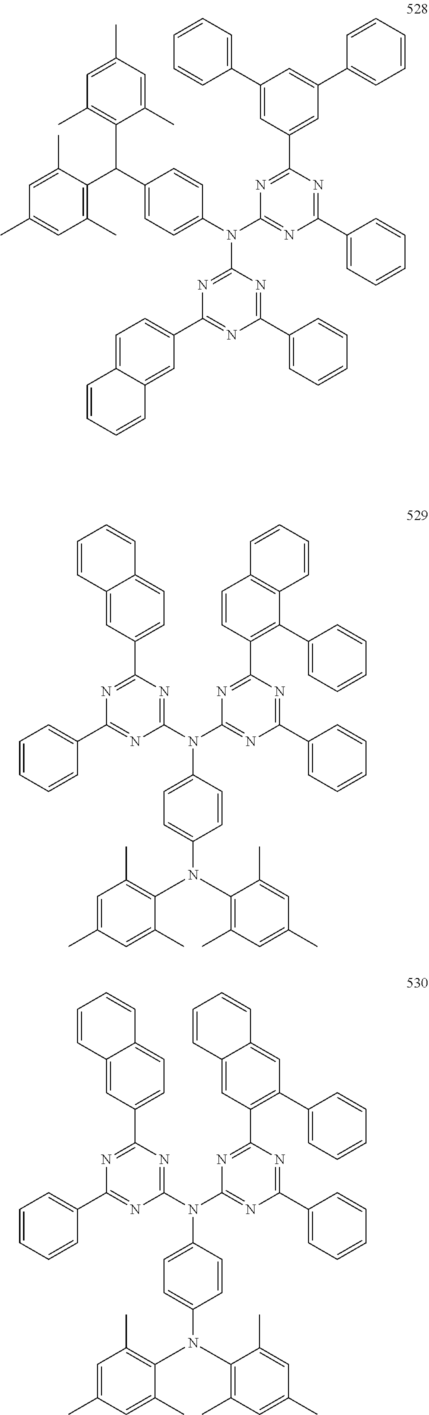 [SMILES: Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4cc5ccccc5cc4-c4ccccc4)n3)cc2)c2c(C)cc(C)cc2C)c(C)c1]

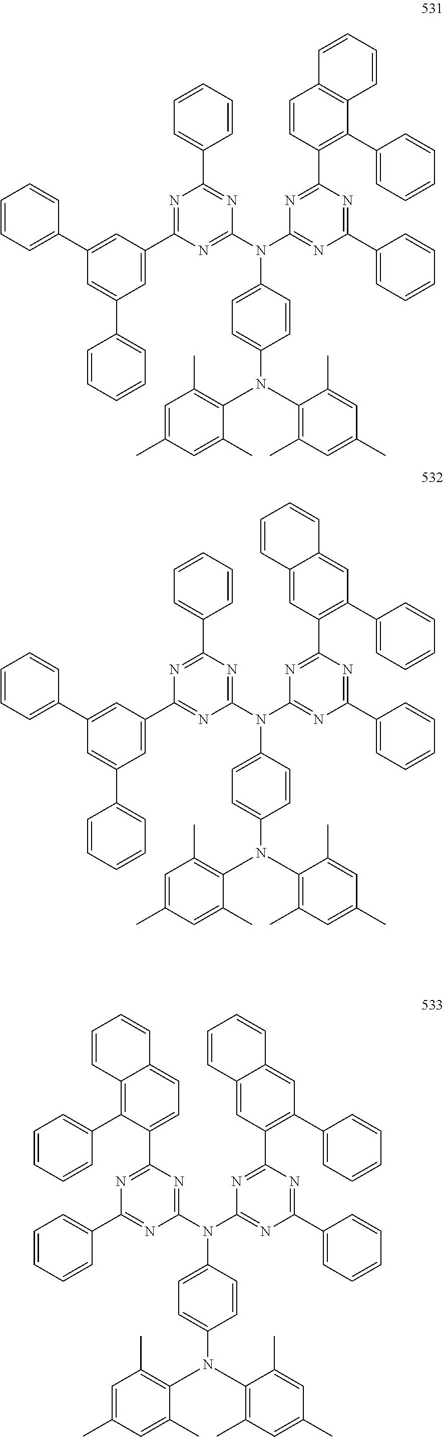 [SMILES: Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4cc5ccccc5cc4-c4ccccc4)n3)c3nc(-c4ccccc4)nc(-c4ccc5ccccc5c4-c4ccccc4)n3)cc2)c2c(C)cc(C)cc2C)c(C)c1]

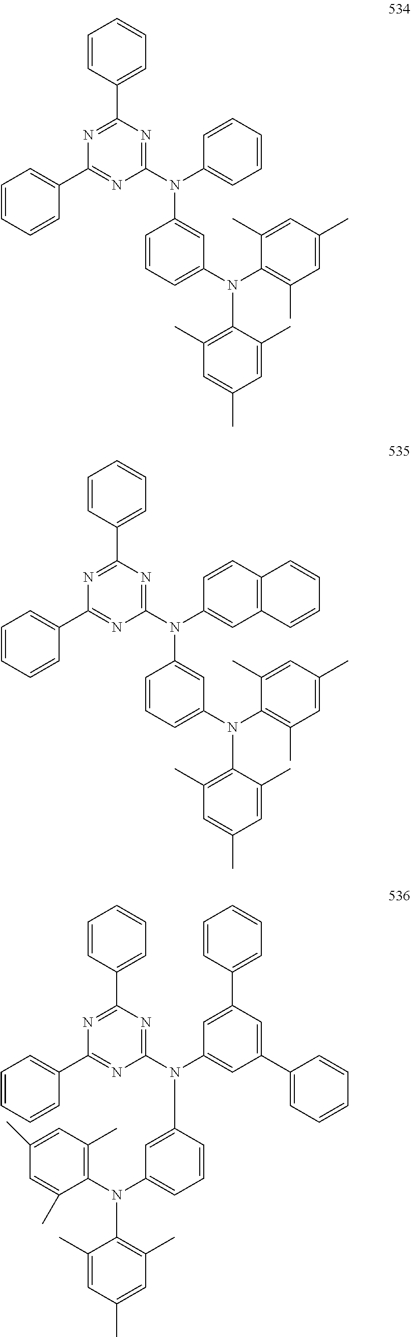

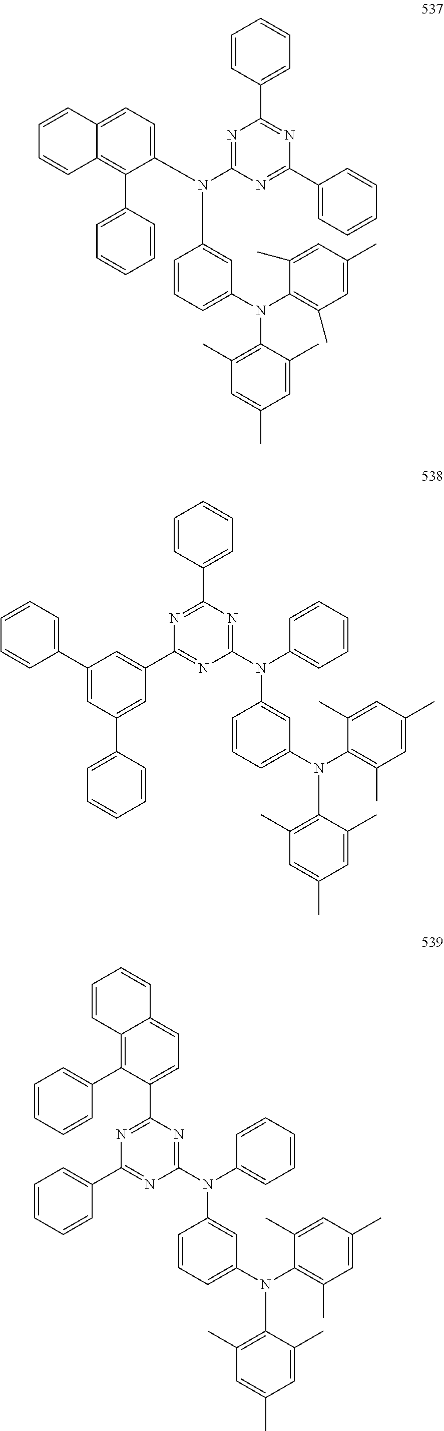 [SMILES: Cc1cc(C)c(N(c2cccc(N(c3nc(-c4ccccc4)nc(-c4ccccc4)n3)c3ccc4ccccc4c3-c3ccccc3)c2)c2c(C)cc(C)cc2C)c(C)c1 ; Cc1cc(C)c(N(c2cccc(N(c3ccccc3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4-c4ccccc4)n3)c2)c2c(C)cc(C)cc2C)c(C)c1]

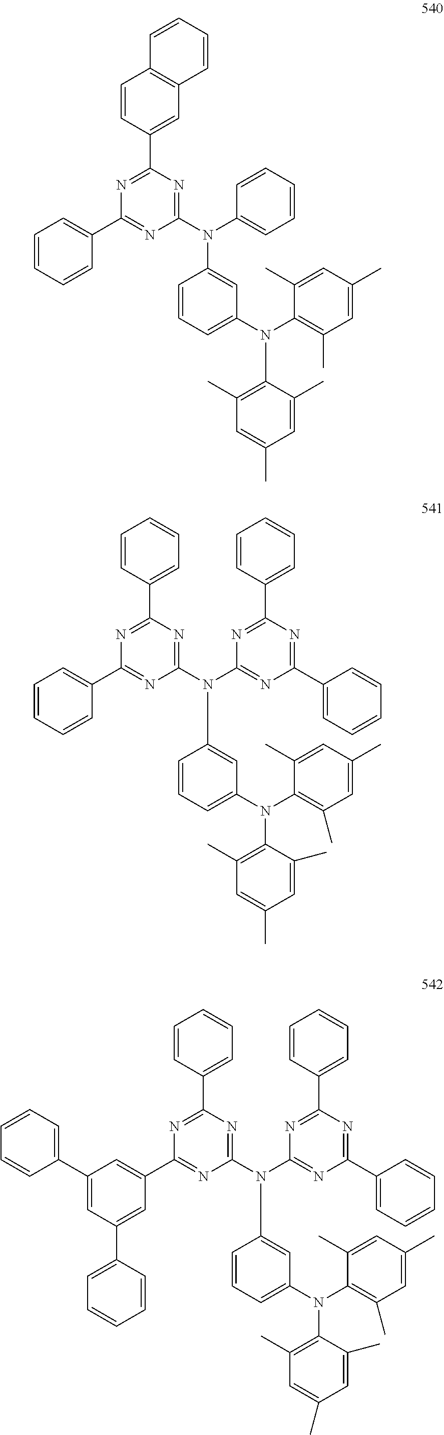

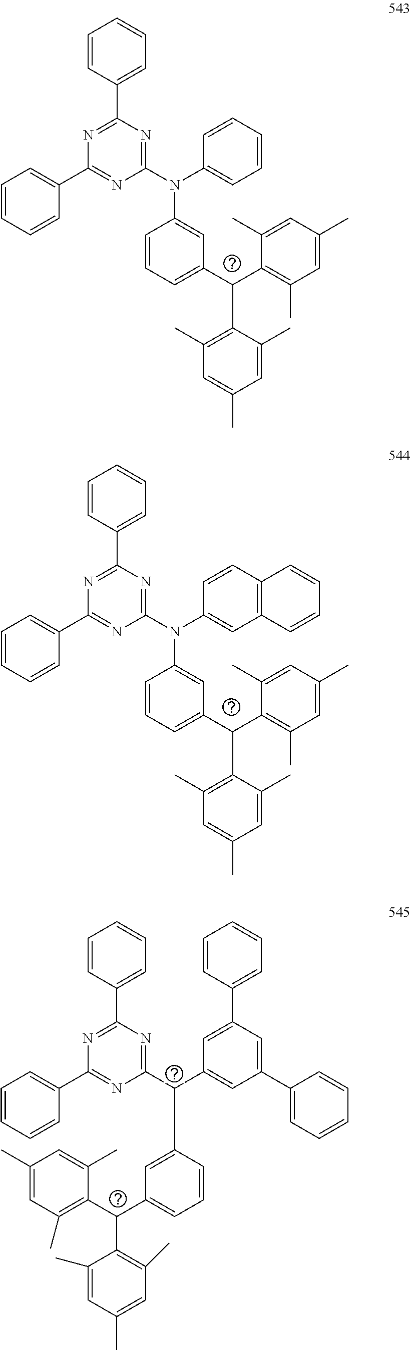 [SMILES: Cc1cc(C)c(C(c2cccc(N(c3ccccc3)c3nc(-c4ccccc4)nc(-c4ccccc4)n3)c2)c2c(C)cc(C)cc2C)c(C)c1 ; Cc1cc(C)c(C(c2cccc(N(c3ccc4ccccc4c3)c3nc(-c4ccccc4)nc(-c4ccccc4)n3)c2)c2c(C)cc(C)cc2C)c(C)c1 ; Cc1cc(C)c(C(c2cccc(C(c3cc(-c4ccccc4)cc(-c4ccccc4)c3)c3nc(-c4ccccc4)nc(-c4ccccc4)n3)c2)c2c(C)cc(C)cc2C)c(C)c1]

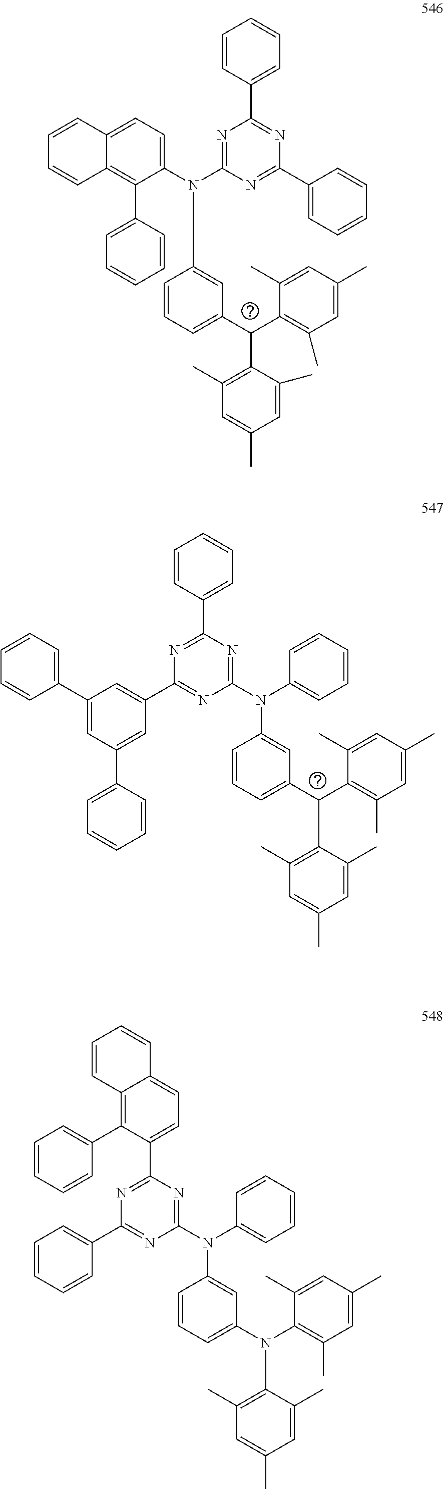 [SMILES: Cc1cc(C)c(C(c2cccc(N(c3nc(-c4ccccc4)nc(-c4ccccc4)n3)c3ccc4ccccc4c3-c3ccccc3)c2)c2c(C)cc(C)cc2C)c(C)c1 ; Cc1cc(C)c(C(c2cccc(N(c3ccccc3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4-c4ccccc4)n3)c2)c2c(C)cc(C)cc2C)c(C)c1]

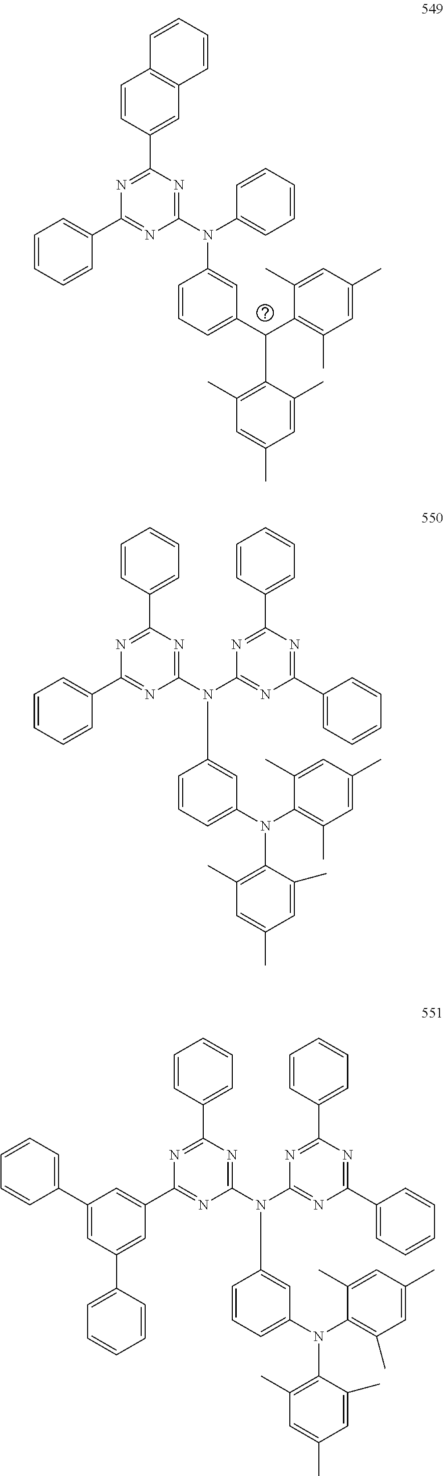

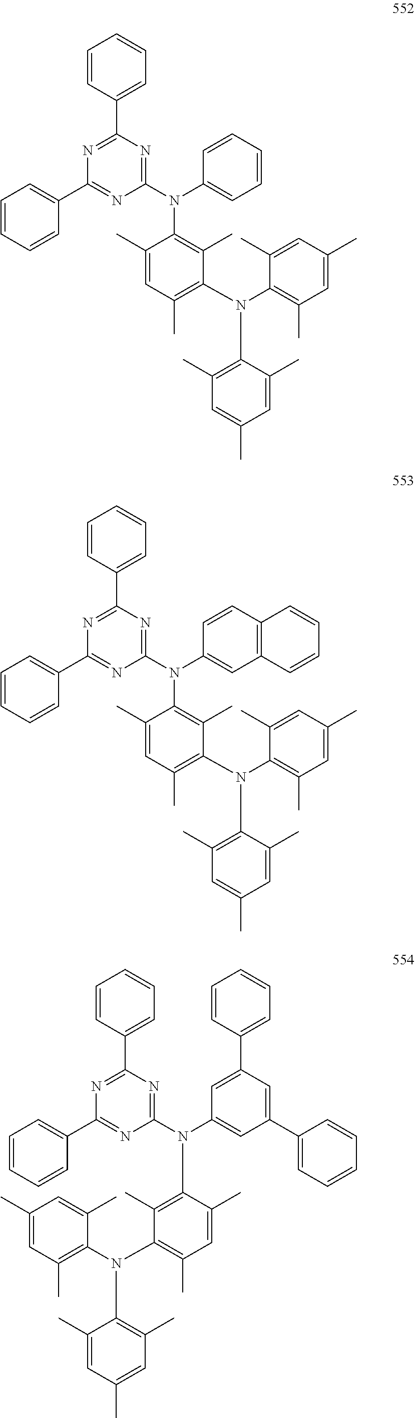

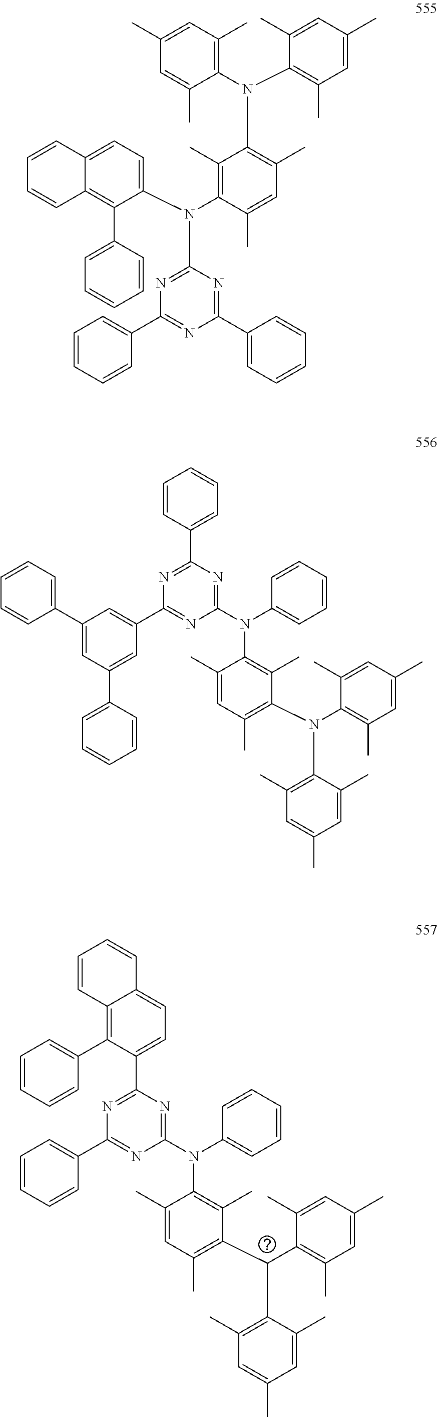 [SMILES: Cc1cc(C)c(N(c2c(C)cc(C)cc2C)c2c(C)cc(C)c(N(c3nc(-c4ccccc4)nc(-c4ccccc4)n3)c3ccc4ccccc4c3-c3ccccc3)c2C)c(C)c1 ; Cc1cc(C)c(N(c2c(C)cc(C)cc2C)c2c(C)cc(C)c(N(c3ccccc3)c3nc(-c4ccccc4)nc(-c4cc(-c5ccccc5)cc(-c5ccccc5)c4)n3)c2C)c(C)c1 ; Cc1cc(C)c(C(c2c(C)cc(C)cc2C)c2c(C)cc(C)c(N(c3ccccc3)c3nc(-c4ccccc4)nc(-c4ccc5ccccc5c4-c4ccccc4)n3)c2C)c(C)c1]

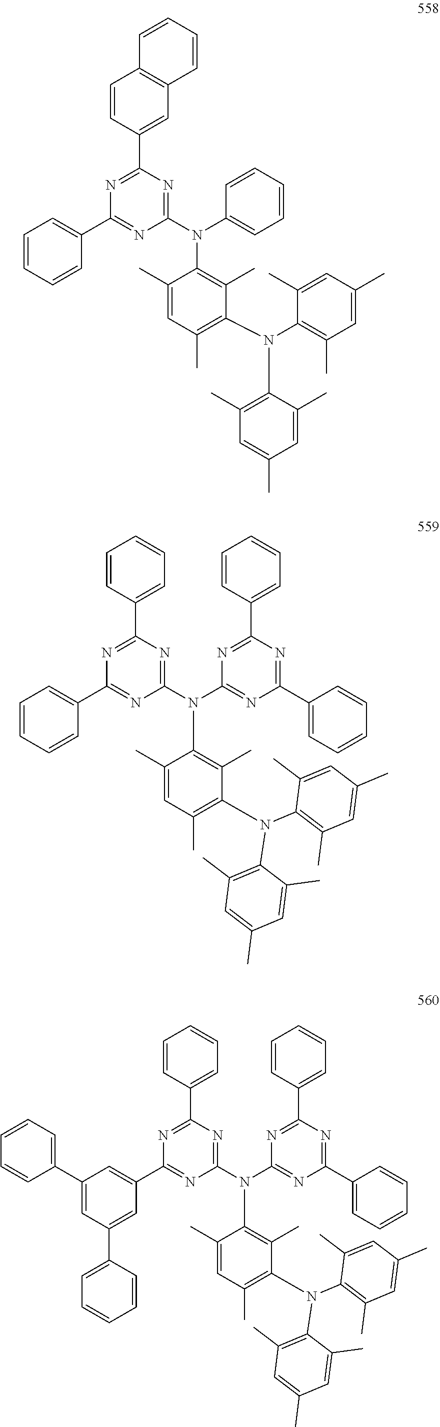 [SMILES: Cc1cc(C)c(N(c2c(C)cc(C)cc2C)c2c(C)cc(C)c(N(c3ccccc3)c3nc(-c4ccccc4)nc(-c4ccc5ccccc5c4)n3)c2C)c(C)c1 ; Cc1cc(C)c(N(c2c(C)cc(C)cc2C)c2c(C)cc(C)c(N(c3nc(-c4ccccc4)nc(-c4ccccc4)n3)c3nc(-c4ccccc4)nc(-c4ccccc4)n3)c2C)c(C)c1 ; Cc1cc(C)c(N(c2c(C)cc(C)cc2C)c2c(C)cc(C)c(N(c3nc(-c4ccccc4)nc(-c4ccccc4)n3)c3nc(-c4ccccc4)nc(-c4cc(-c5ccccc5)cc(-c5ccccc5)c4)n3)c2C)c(C)c1]

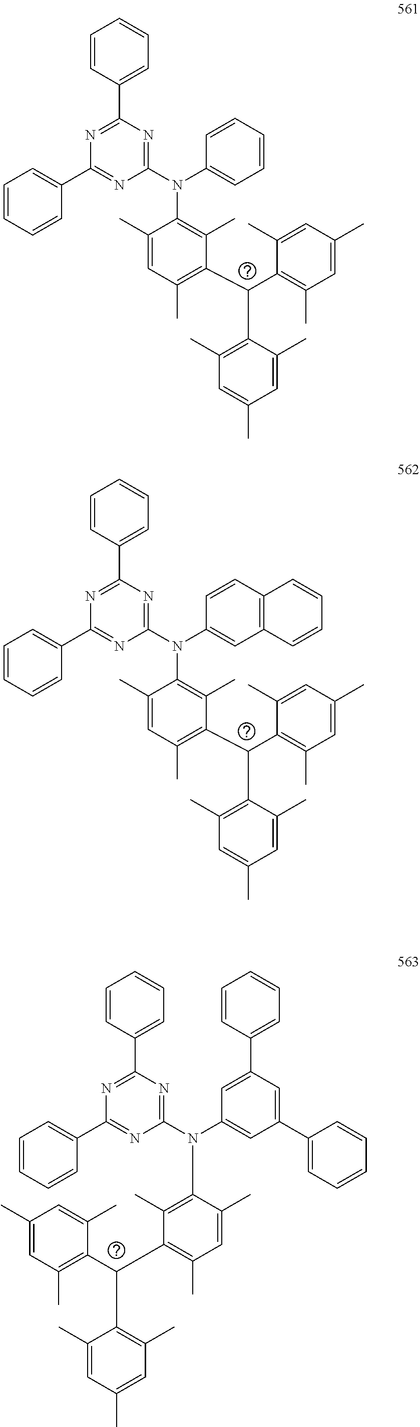

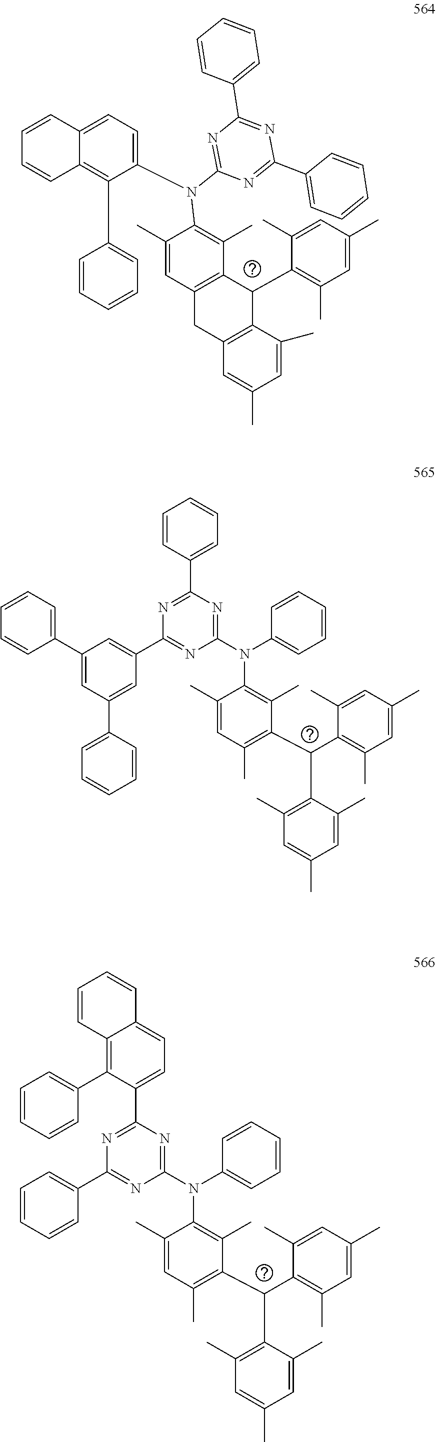 [SMILES: Cc1cc(C)c(C2c3c(C)cc(C)cc3Cc3cc(C)c(N(c4nc(-c5ccccc5)nc(-c5ccccc5)n4)c4ccc5ccccc5c4-c4ccccc4)c(C)c32)c(C)c1 ; Cc1cc(C)c(C(c2c(C)cc(C)cc2C)c2c(C)cc(C)c(N(c3ccccc3)c3nc(-c4ccccc4)nc(-c4cc(-c5ccccc5)cc(-c5ccccc5)c4)n3)c2C)c(C)c1 ; Cc1cc(C)c(C(c2c(C)cc(C)cc2C)c2c(C)cc(C)c(N(c3ccccc3)c3nc(-c4ccccc4)nc(-c4ccc5ccccc5c4-c4ccccc4)n3)c2C)c(C)c1]

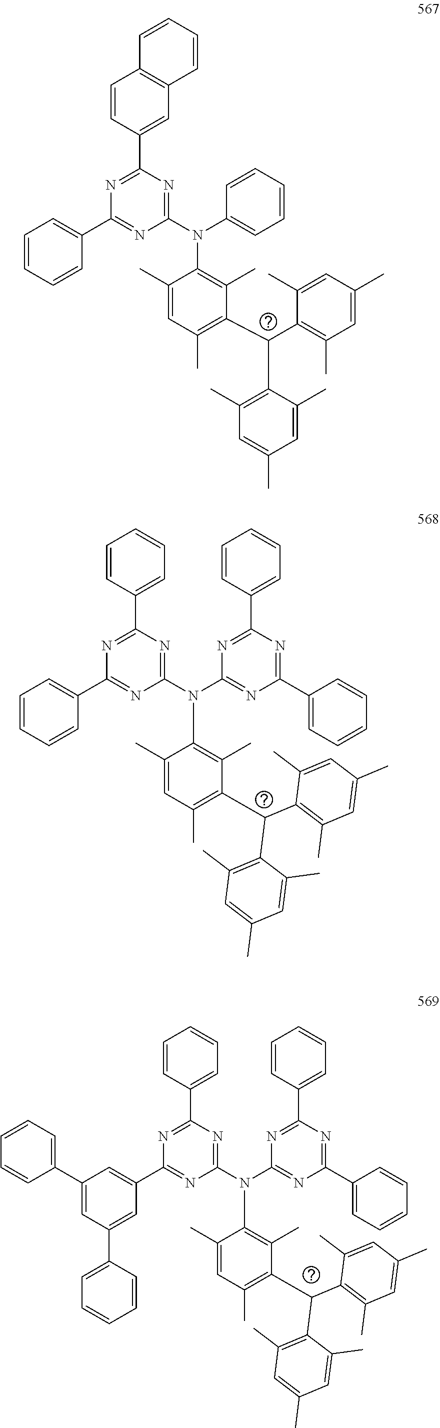

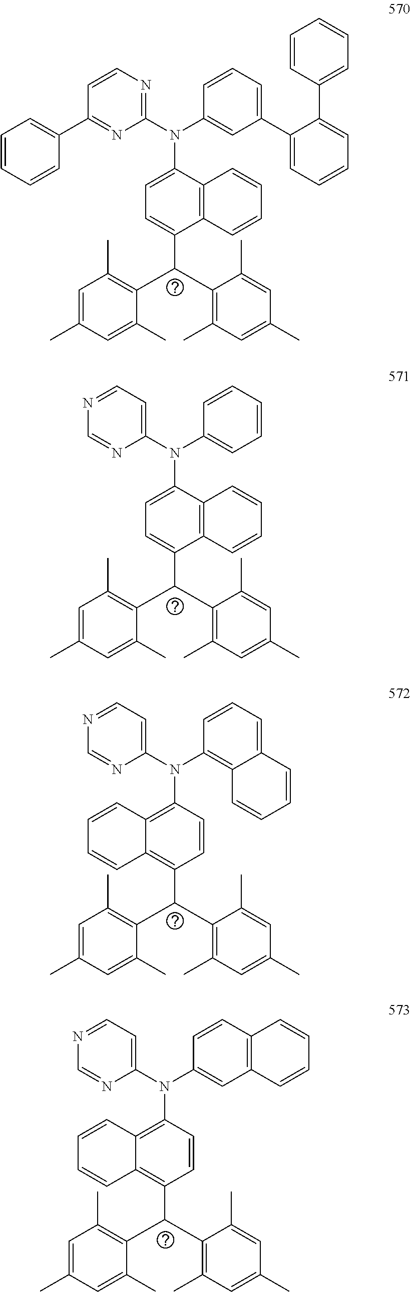

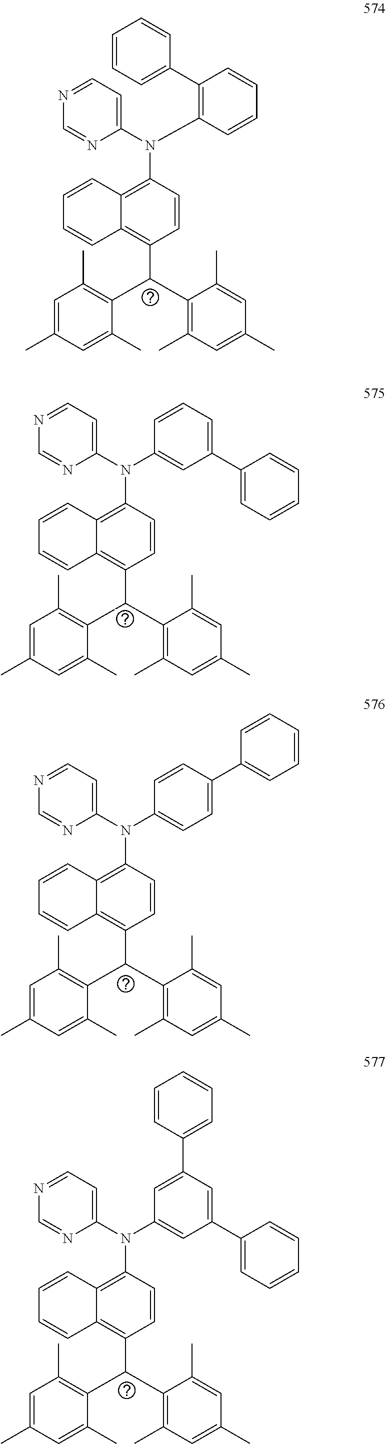

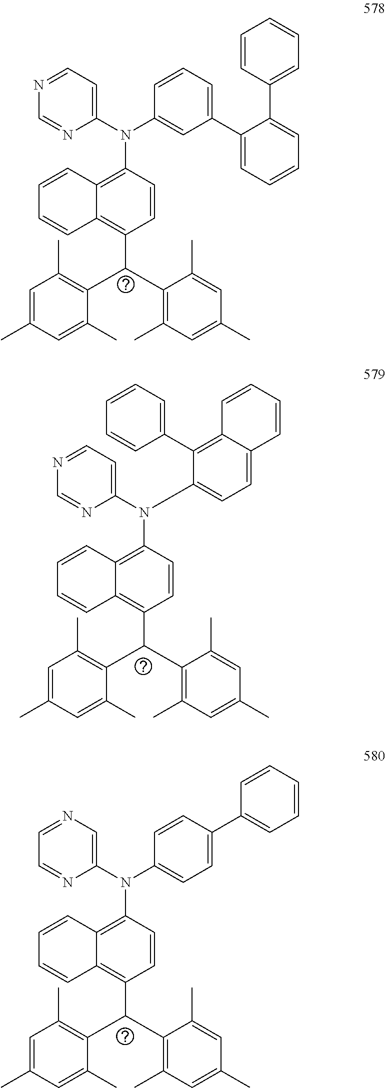

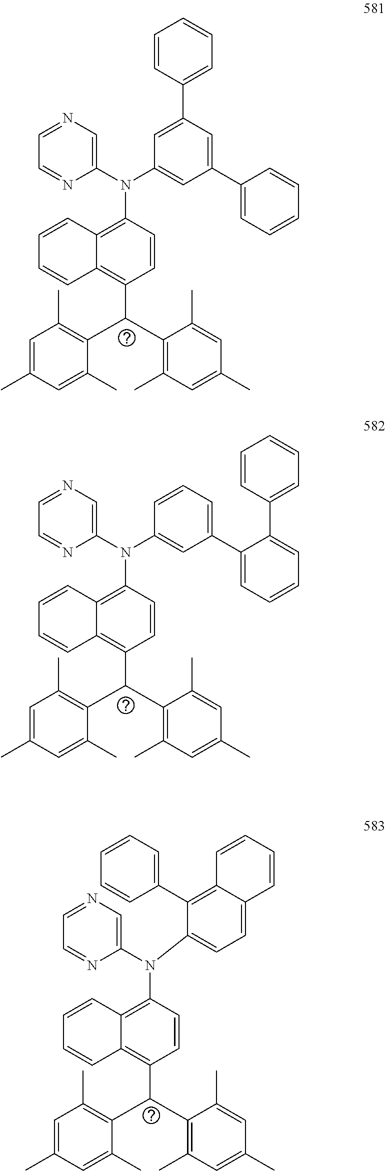

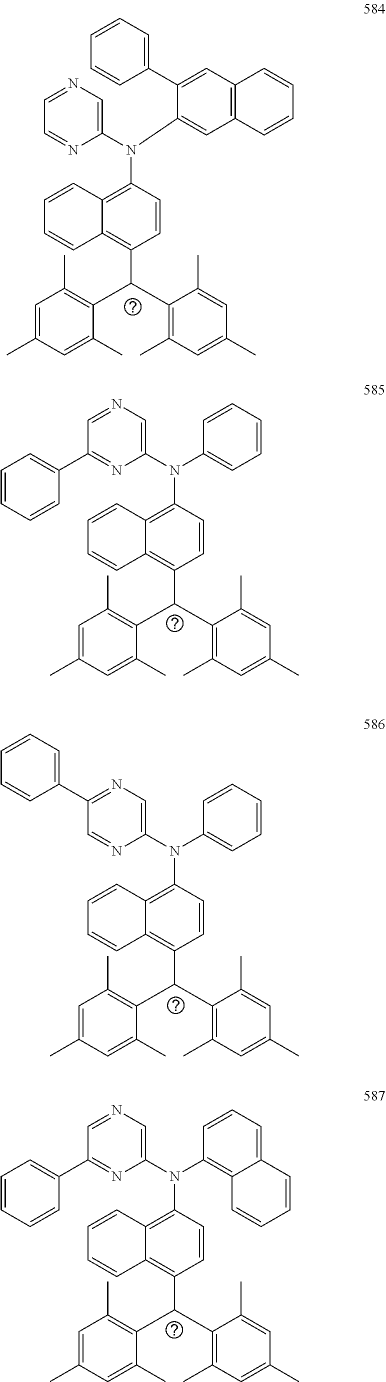 [SMILES: Cc1cc(C)c(C(c2c(C)cc(C)cc2C)c2ccc(N(c3cnccn3)c3cc4ccccc4cc3-c3ccccc3)c3ccccc23)c(C)c1 ; Cc1cc(C)c(C(c2c(C)cc(C)cc2C)c2ccc(N(c3ccccc3)c3cncc(-c4ccccc4)n3)c3ccccc23)c(C)c1 ; Cc1cc(C)c(C(c2c(C)cc(C)cc2C)c2ccc(N(c3ccccc3)c3cnc(-c4ccccc4)cn3)c3ccccc23)c(C)c1 ; Cc1cc(C)c(C(c2c(C)cc(C)cc2C)c2ccc(N(c3cncc(-c4ccccc4)n3)c3cccc4ccccc34)c3ccccc23)c(C)c1]

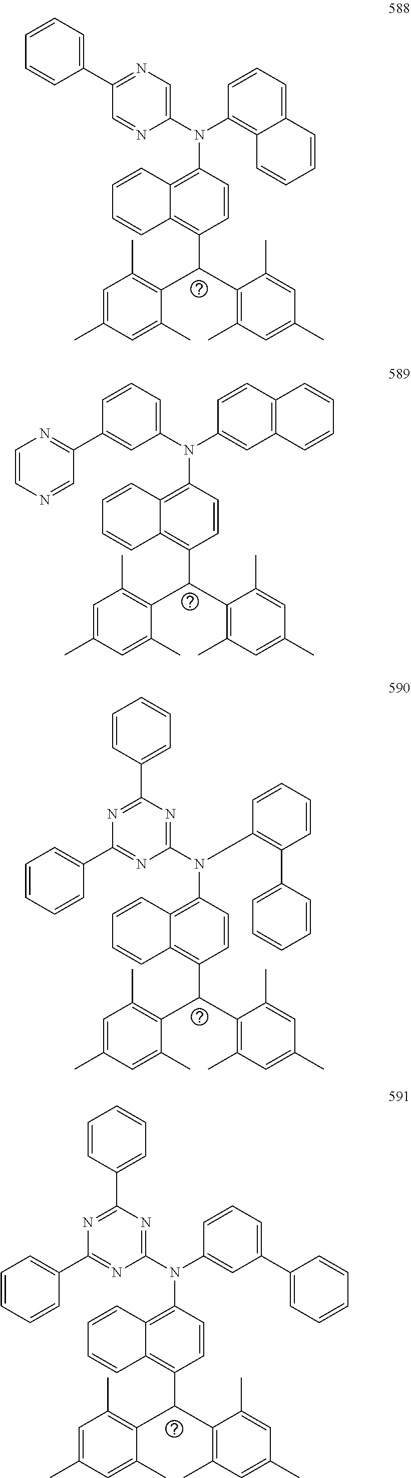

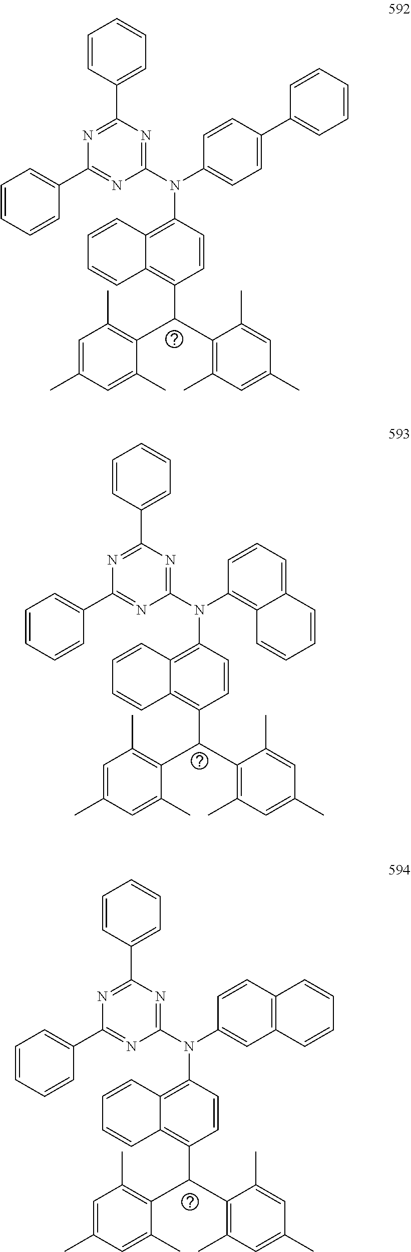 [SMILES: Cc1cc(C)c(C(c2c(C)cc(C)cc2C)c2ccc(N(c3ccc(-c4ccccc4)cc3)c3nc(-c4ccccc4)nc(-c4ccccc4)n3)c3ccccc23)c(C)c1 ; Cc1cc(C)c(C(c2c(C)cc(C)cc2C)c2ccc(N(c3nc(-c4ccccc4)nc(-c4ccccc4)n3)c3cccc4ccccc34)c3ccccc23)c(C)c1 ; Cc1cc(C)c(C(c2c(C)cc(C)cc2C)c2ccc(N(c3ccc4ccccc4c3)c3nc(-c4ccccc4)nc(-c4ccccc4)n3)c3ccccc23)c(C)c1]

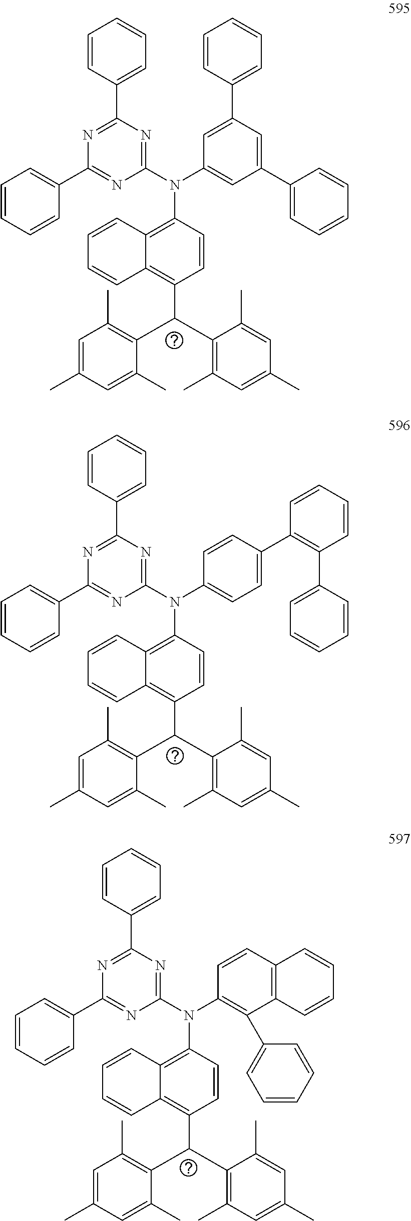

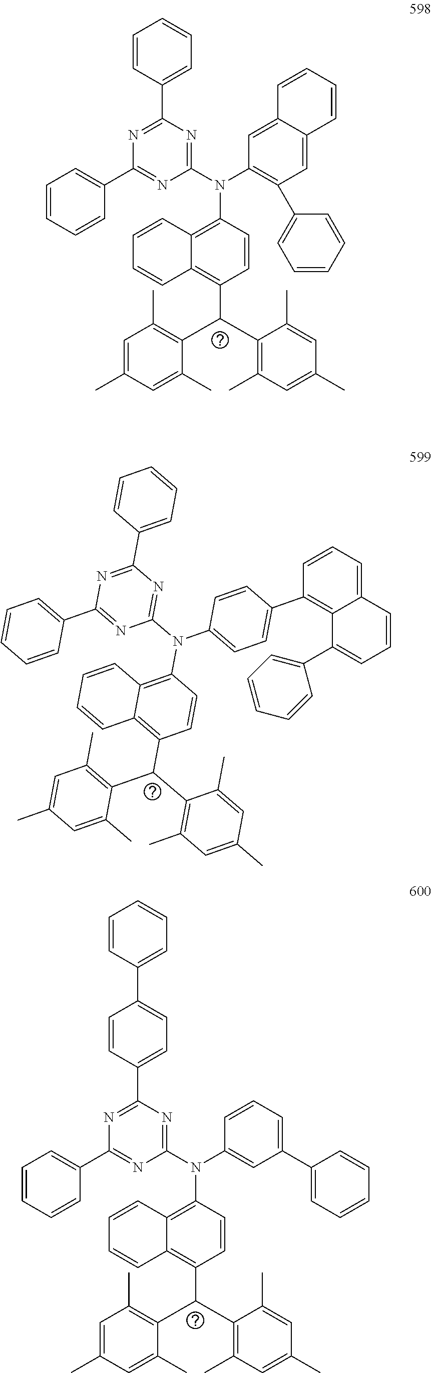 [SMILES: Cc1cc(C)c(C(c2c(C)cc(C)cc2C)c2ccc(N(c3nc(-c4ccccc4)nc(-c4ccccc4)n3)c3cc4ccccc4cc3-c3ccccc3)c3ccccc23)c(C)c1 ; Cc1cc(C)c(C(c2c(C)cc(C)cc2C)c2ccc(N(c3ccc(-c4cccc5cccc(-c6ccccc6)c45)cc3)c3nc(-c4ccccc4)nc(-c4ccccc4)n3)c3ccccc23)c(C)c1 ; Cc1cc(C)c(C(c2c(C)cc(C)cc2C)c2ccc(N(c3cccc(-c4ccccc4)c3)c3nc(-c4ccccc4)nc(-c4ccc(-c5ccccc5)cc4)n3)c3ccccc23)c(C)c1]

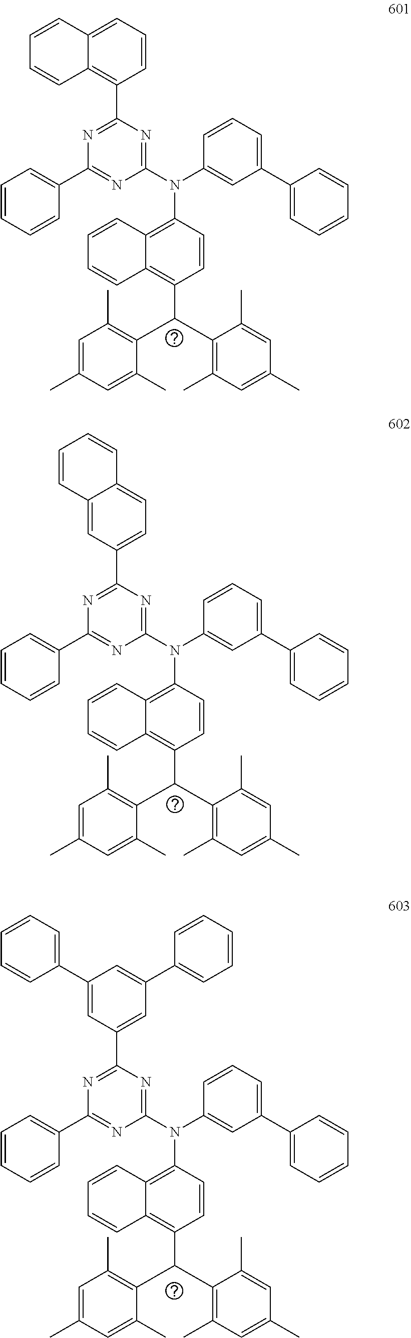 [SMILES: Cc1cc(C)c(C(c2c(C)cc(C)cc2C)c2ccc(N(c3cccc(-c4ccccc4)c3)c3nc(-c4ccccc4)nc(-c4cccc5ccccc45)n3)c3ccccc23)c(C)c1 ; Cc1cc(C)c(C(c2c(C)cc(C)cc2C)c2ccc(N(c3cccc(-c4ccccc4)c3)c3nc(-c4ccccc4)nc(-c4ccc5ccccc5c4)n3)c3ccccc23)c(C)c1 ; Cc1cc(C)c(C(c2c(C)cc(C)cc2C)c2ccc(N(c3cccc(-c4ccccc4)c3)c3nc(-c4ccccc4)nc(-c4cc(-c5ccccc5)cc(-c5ccccc5)c4)n3)c3ccccc23)c(C)c1]

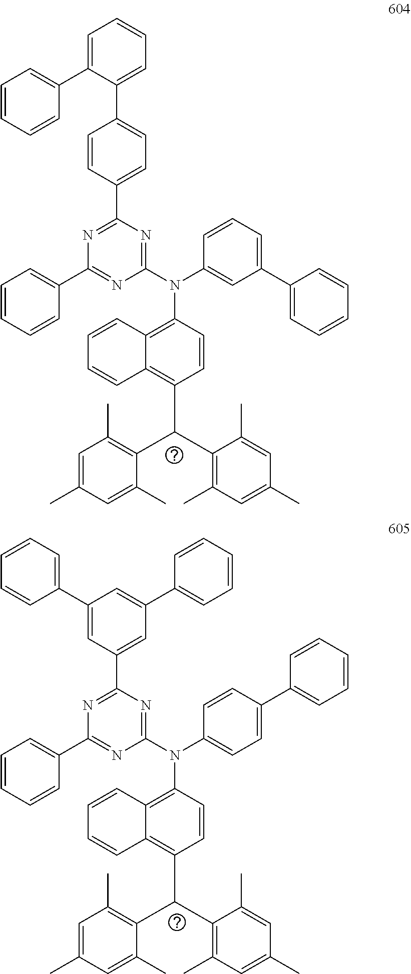

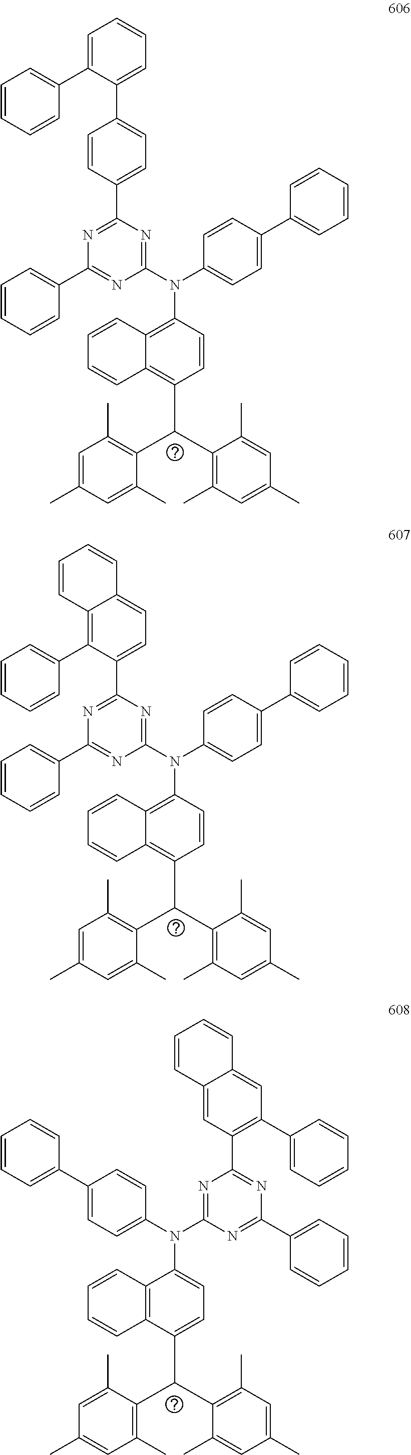

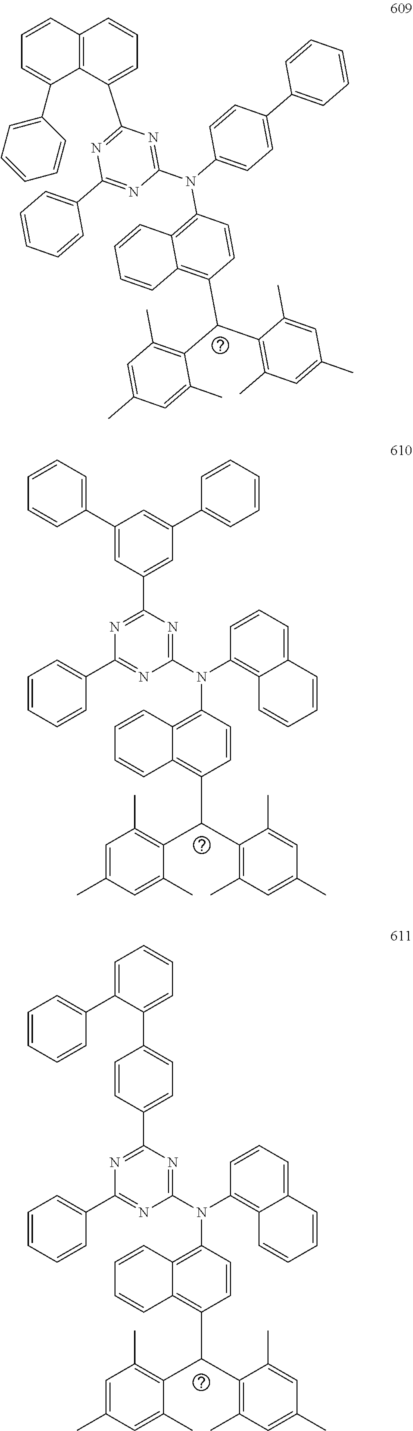 [SMILES: Cc1cc(C)c(C(c2c(C)cc(C)cc2C)c2ccc(N(c3ccc(-c4ccccc4)cc3)c3nc(-c4ccccc4)nc(-c4cccc5cccc(-c6ccccc6)c45)n3)c3ccccc23)c(C)c1 ; Cc1cc(C)c(C(c2c(C)cc(C)cc2C)c2ccc(N(c3nc(-c4ccccc4)nc(-c4cc(-c5ccccc5)cc(-c5ccccc5)c4)n3)c3cccc4ccccc34)c3ccccc23)c(C)c1 ; Cc1cc(C)c(C(c2c(C)cc(C)cc2C)c2ccc(N(c3nc(-c4ccccc4)nc(-c4ccc(-c5ccccc5-c5ccccc5)cc4)n3)c3cccc4ccccc34)c3ccccc23)c(C)c1]

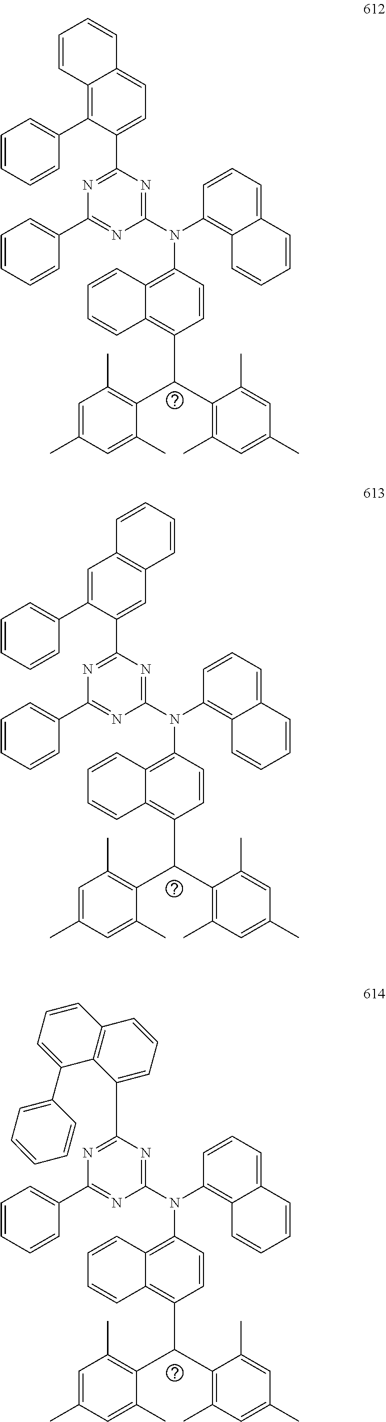 [SMILES: Cc1cc(C)c(C(c2c(C)cc(C)cc2C)c2ccc(N(c3nc(-c4ccccc4)nc(-c4ccc5ccccc5c4-c4ccccc4)n3)c3cccc4ccccc34)c3ccccc23)c(C)c1 ; Cc1cc(C)c(C(c2c(C)cc(C)cc2C)c2ccc(N(c3nc(-c4ccccc4)nc(-c4cc5ccccc5cc4-c4ccccc4)n3)c3cccc4ccccc34)c3ccccc23)c(C)c1 ; Cc1cc(C)c(C(c2c(C)cc(C)cc2C)c2ccc(N(c3nc(-c4ccccc4)nc(-c4cccc5cccc(-c6ccccc6)c45)n3)c3cccc4ccccc34)c3ccccc23)c(C)c1]

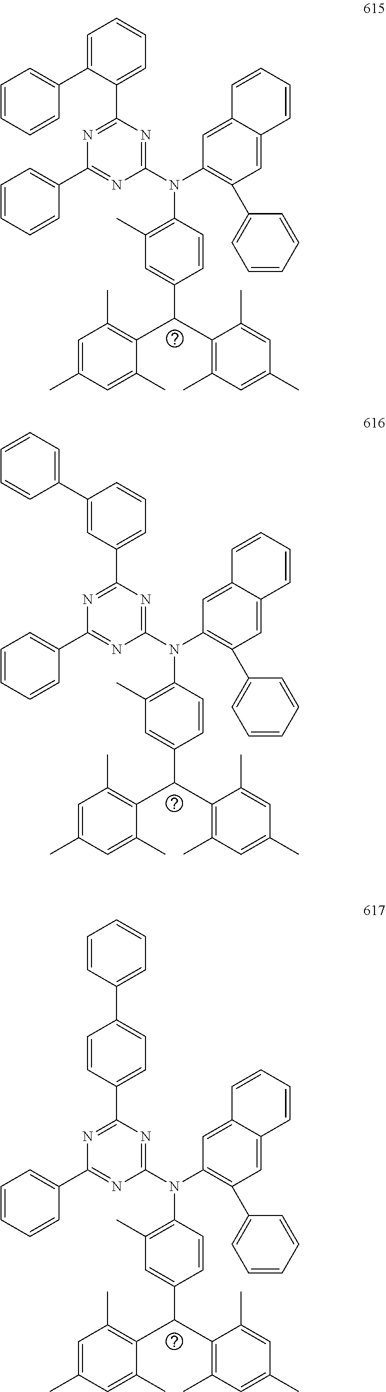 [SMILES: Cc1cc(C)c(C(c2ccc(N(c3nc(-c4ccccc4)nc(-c4ccccc4-c4ccccc4)n3)c3cc4ccccc4cc3-c3ccccc3)c(C)c2)c2c(C)cc(C)cc2C)c(C)c1 ; Cc1cc(C)c(C(c2ccc(N(c3nc(-c4ccccc4)nc(-c4cccc(-c5ccccc5)c4)n3)c3cc4ccccc4cc3-c3ccccc3)c(C)c2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3cc4ccccc4cc3-c3ccccc3)c(C)c2)c2c(C)cc(C)cc2C)c(C)c1]

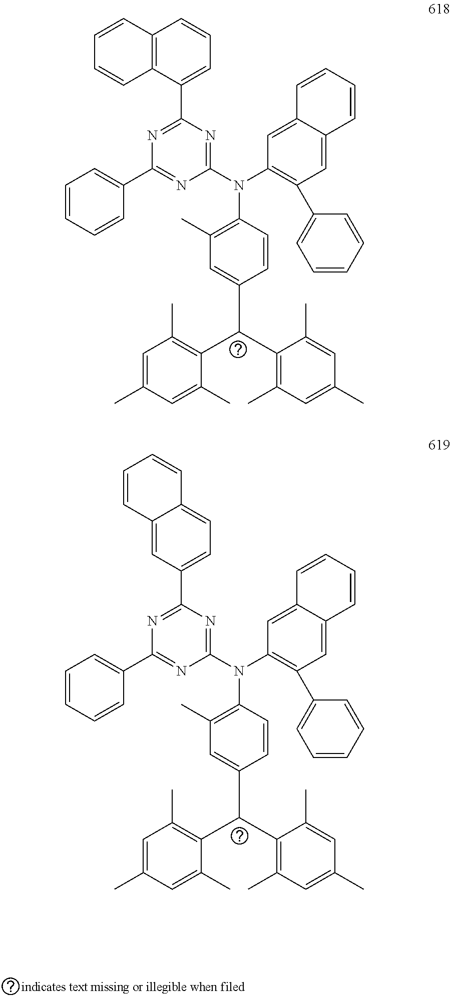 [SMILES: Cc1cc(C)c(C(c2ccc(N(c3nc(-c4ccccc4)nc(-c4cccc5ccccc45)n3)c3cc4ccccc4cc3-c3ccccc3)c(C)c2)c2c(C)cc(C)cc2C)c(C)c1 ; Cc1cc(C)c(C(c2ccc(N(c3nc(-c4ccccc4)nc(-c4ccc5ccccc5c4)n3)c3cc4ccccc4cc3-c3ccccc3)c(C)c2)c2c(C)cc(C)cc2C)c(C)c1]

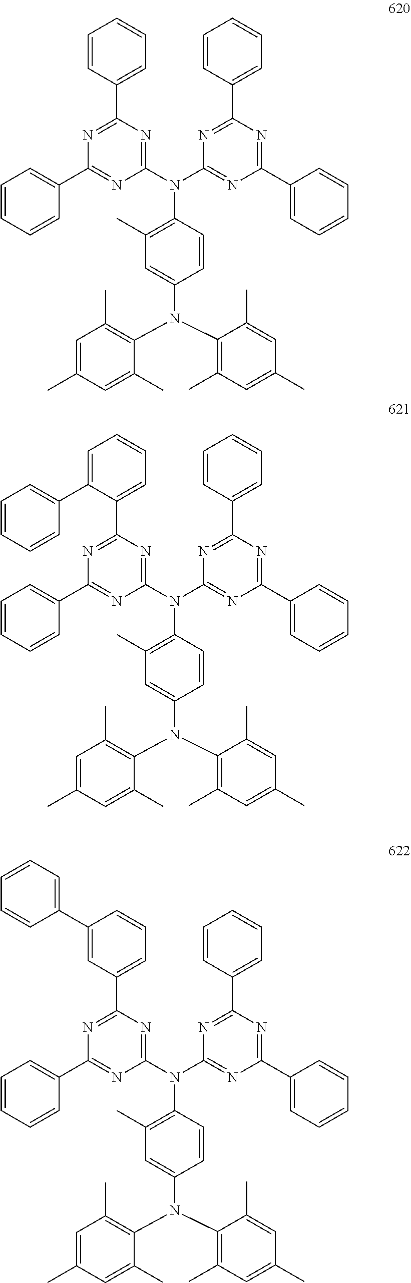

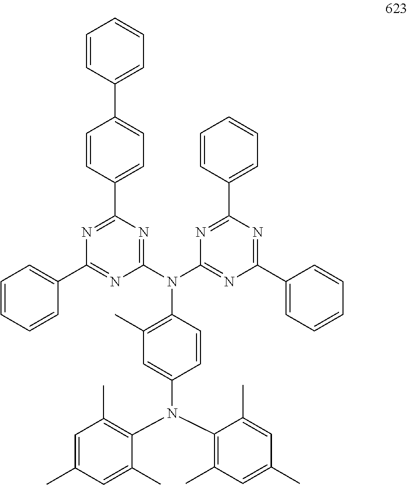 [SMILES: Cc1cc(C)c(N(c2ccc(N(c3nc(-c4ccccc4)nc(-c4ccccc4)n3)c3nc(-c4ccccc4)nc(-c4ccc(-c5ccccc5)cc4)n3)c(C)c2)c2c(C)cc(C)cc2C)c(C)c1]

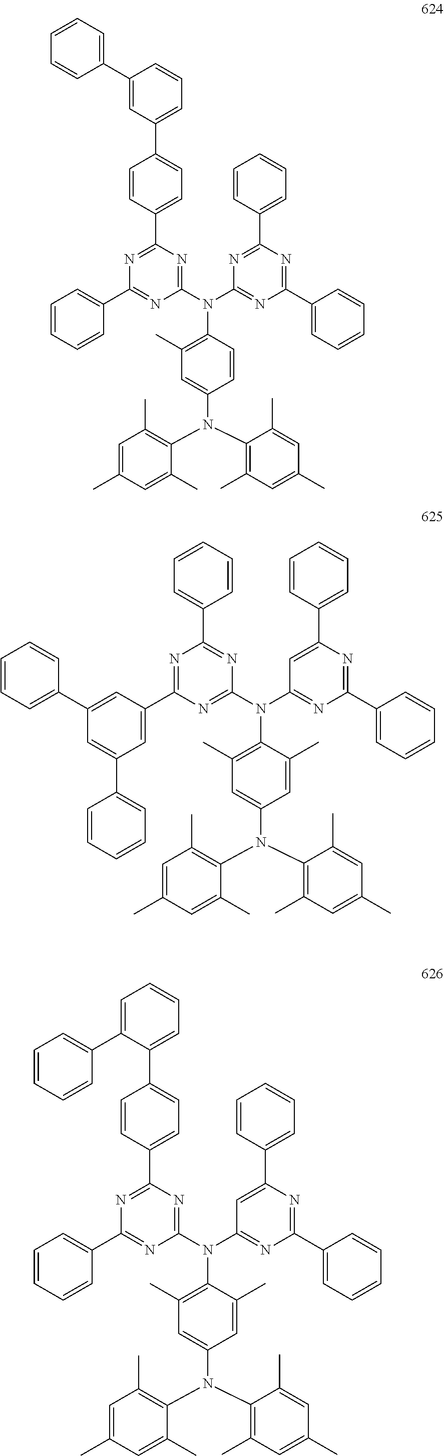 [SMILES: Cc1cc(C)c(N(c2ccc(N(c3nc(-c4ccccc4)nc(-c4ccccc4)n3)c3nc(-c4ccccc4)nc(-c4ccc(-c5cccc(-c6ccccc6)c5)cc4)n3)c(C)c2)c2c(C)cc(C)cc2C)c(C)c1 ; Cc1cc(C)c(N(c2cc(C)c(N(c3cc(-c4ccccc4)nc(-c4ccccc4)n3)c3nc(-c4ccccc4)nc(-c4cc(-c5ccccc5)cc(-c5ccccc5)c4)n3)c(C)c2)c2c(C)cc(C)cc2C)c(C)c1 ; Cc1cc(C)c(N(c2cc(C)c(N(c3cc(-c4ccccc4)nc(-c4ccccc4)n3)c3nc(-c4ccccc4)nc(-c4ccc(-c5ccccc5-c5ccccc5)cc4)n3)c(C)c2)c2c(C)cc(C)cc2C)c(C)c1]

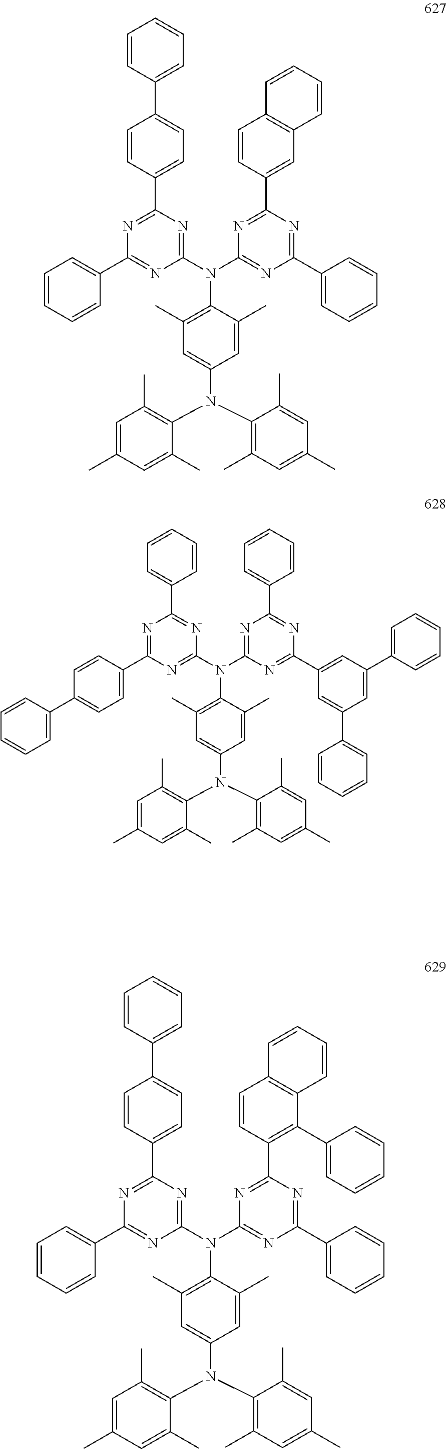 [SMILES: Cc1cc(C)c(N(c2cc(C)c(N(c3nc(-c4ccccc4)nc(-c4ccc(-c5ccccc5)cc4)n3)c3nc(-c4ccccc4)nc(-c4ccc5ccccc5c4)n3)c(C)c2)c2c(C)cc(C)cc2C)c(C)c1 ; Cc1cc(C)c(N(c2cc(C)c(N(c3nc(-c4ccccc4)nc(-c4ccc(-c5ccccc5)cc4)n3)c3nc(-c4ccccc4)nc(-c4cc(-c5ccccc5)cc(-c5ccccc5)c4)n3)c(C)c2)c2c(C)cc(C)cc2C)c(C)c1 ; Cc1cc(C)c(N(c2cc(C)c(N(c3nc(-c4ccccc4)nc(-c4ccc(-c5ccccc5)cc4)n3)c3nc(-c4ccccc4)nc(-c4ccc5ccccc5c4-c4ccccc4)n3)c(C)c2)c2c(C)cc(C)cc2C)c(C)c1]

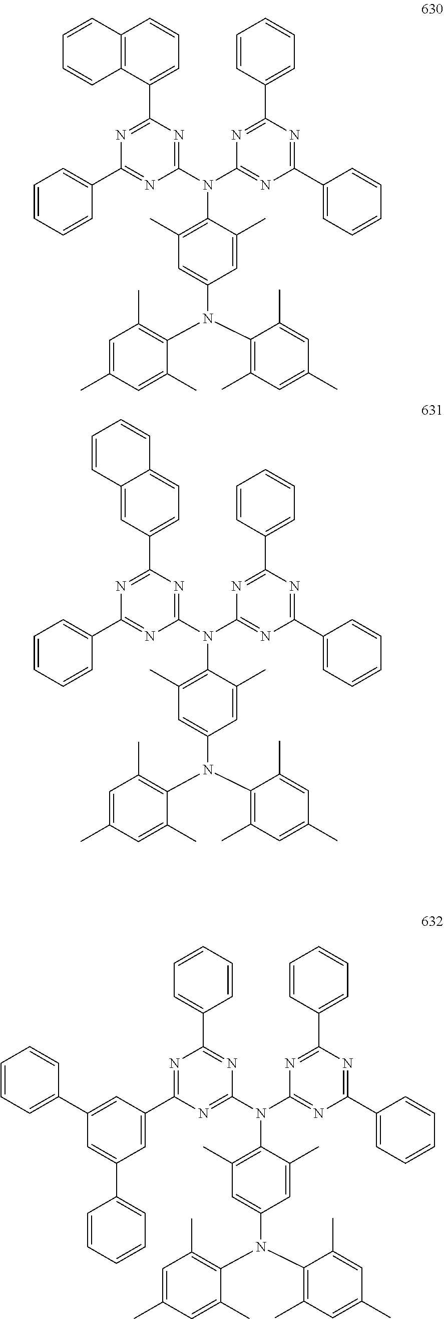

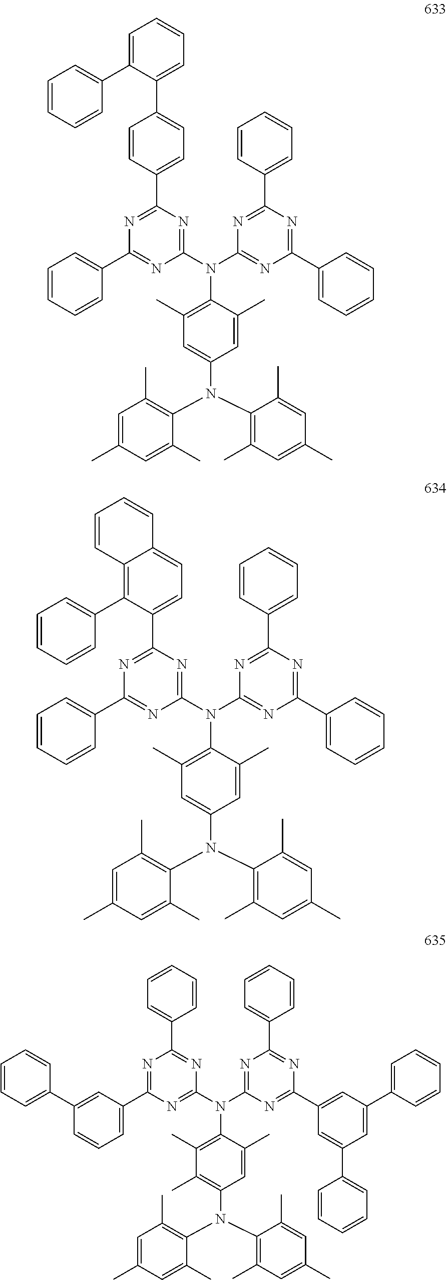

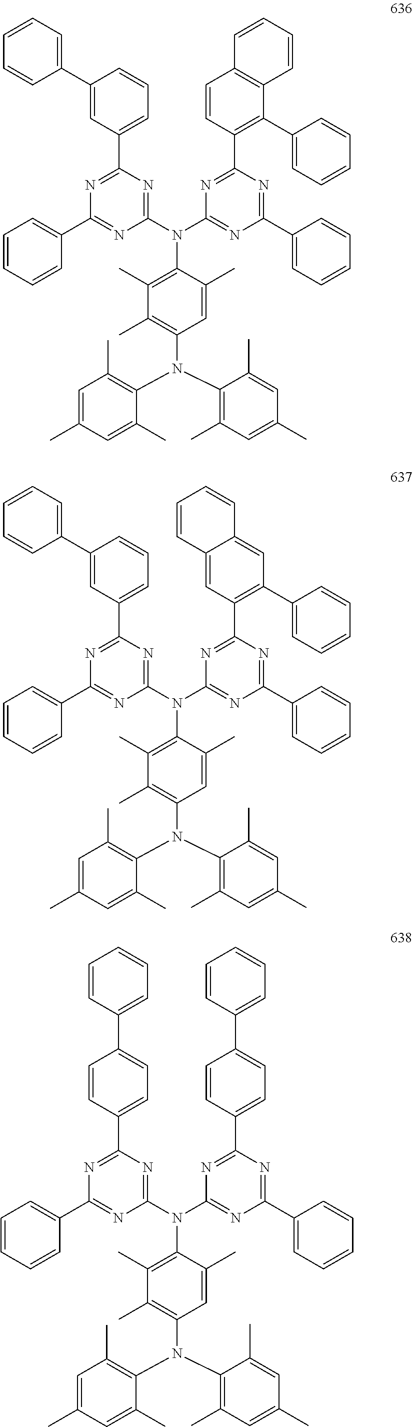

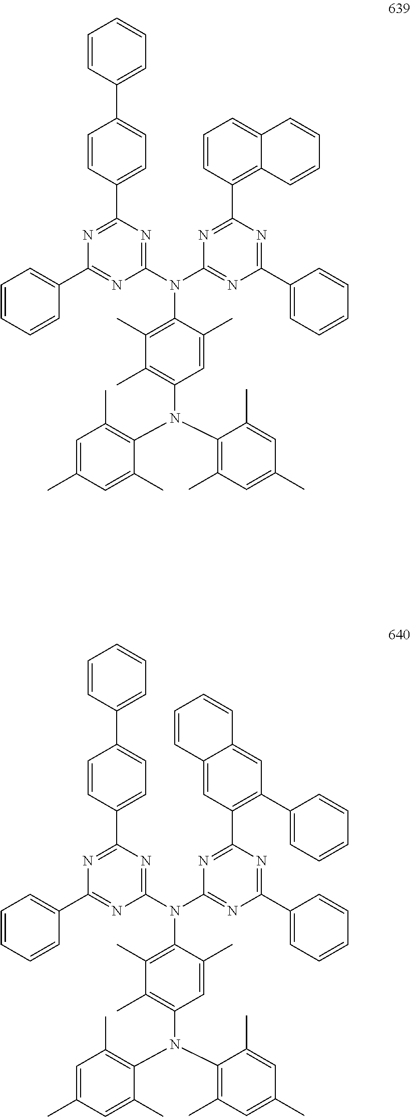 [SMILES: Cc1cc(C)c(N(c2cc(C)c(N(c3nc(-c4ccccc4)nc(-c4ccc(-c5ccccc5)cc4)n3)c3nc(-c4ccccc4)nc(-c4cccc5ccccc45)n3)c(C)c2C)c2c(C)cc(C)cc2C)c(C)c1 ; Cc1cc(C)c(N(c2cc(C)c(N(c3nc(-c4ccccc4)nc(-c4ccc(-c5ccccc5)cc4)n3)c3nc(-c4ccccc4)nc(-c4cc5ccccc5cc4-c4ccccc4)n3)c(C)c2C)c2c(C)cc(C)cc2C)c(C)c1]

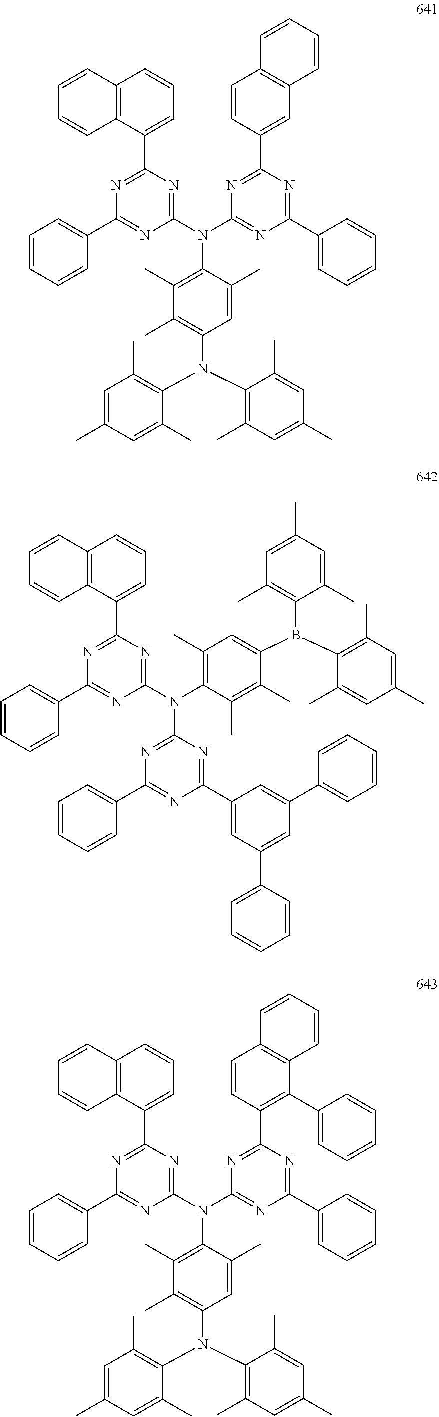 [SMILES: Cc1cc(C)c(N(c2cc(C)c(N(c3nc(-c4ccccc4)nc(-c4ccc5ccccc5c4)n3)c3nc(-c4ccccc4)nc(-c4cccc5ccccc45)n3)c(C)c2C)c2c(C)cc(C)cc2C)c(C)c1 ; Cc1cc(C)c(B(c2cc(C)c(N(c3nc(-c4ccccc4)nc(-c4cc(-c5ccccc5)cc(-c5ccccc5)c4)n3)c3nc(-c4ccccc4)nc(-c4cccc5ccccc45)n3)c(C)c2C)c2c(C)cc(C)cc2C)c(C)c1 ; Cc1cc(C)c(N(c2cc(C)c(N(c3nc(-c4ccccc4)nc(-c4ccc5ccccc5c4-c4ccccc4)n3)c3nc(-c4ccccc4)nc(-c4cccc5ccccc45)n3)c(C)c2C)c2c(C)cc(C)cc2C)c(C)c1]

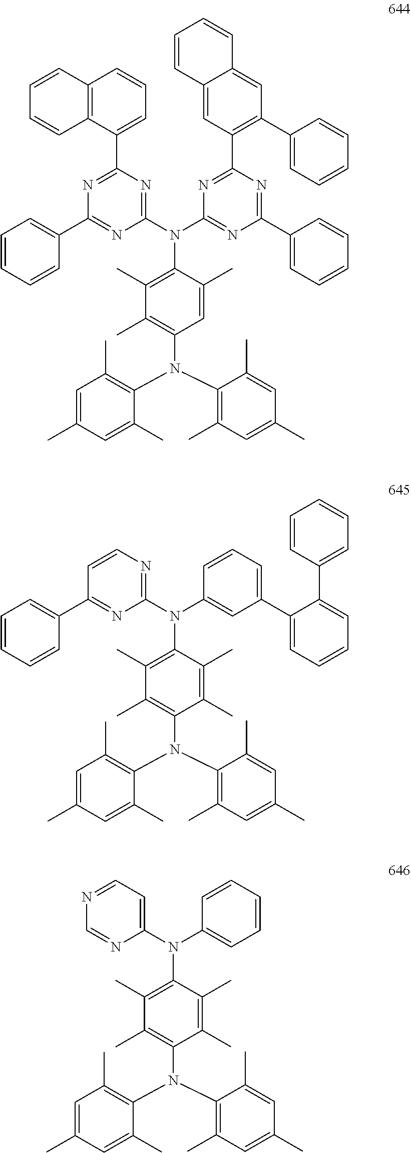

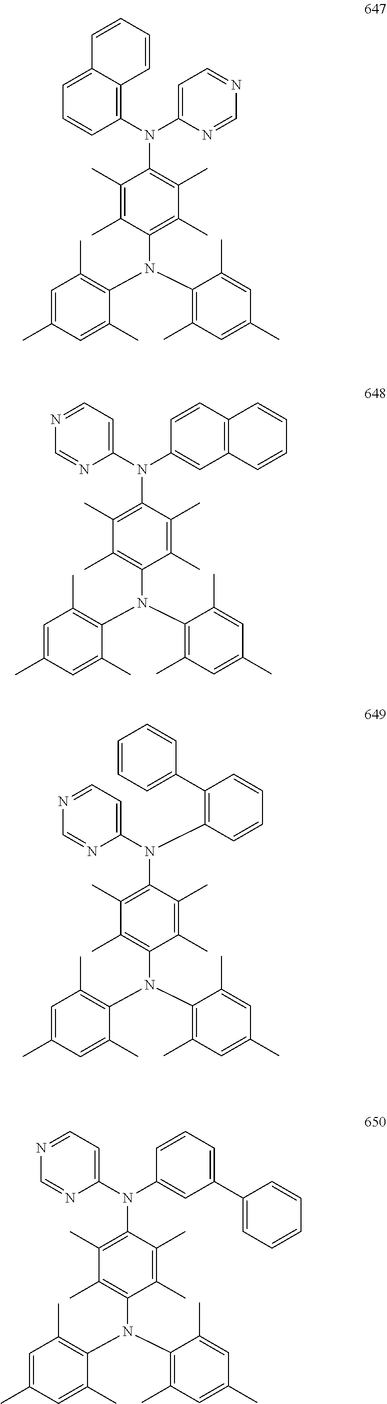

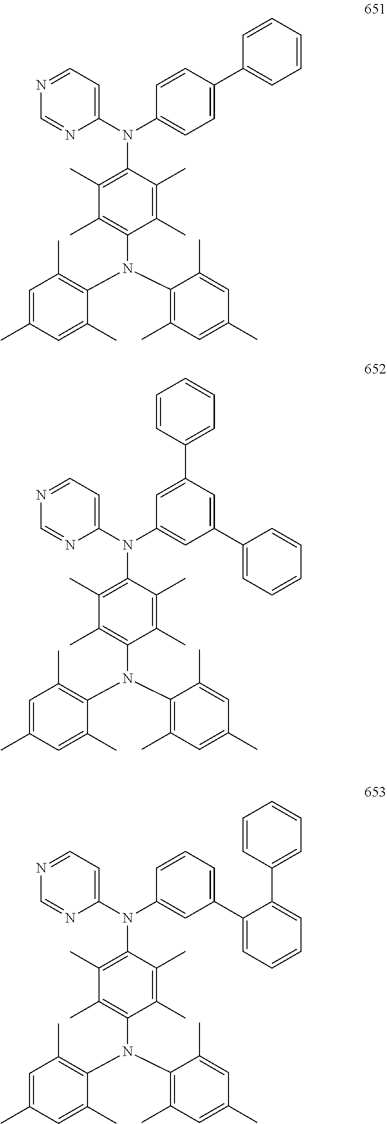

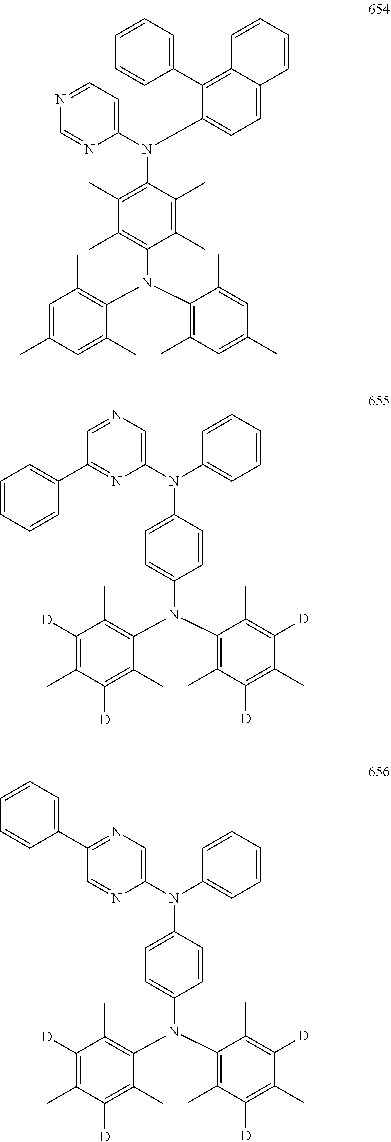 [SMILES: Cc1cc(C)c(N(c2c(C)cc(C)cc2C)c2c(C)c(C)c(N(c3ccncn3)c3ccc4ccccc4c3-c3ccccc3)c(C)c2C)c(C)c1 ; [2H]c1c(C)c([2H])c(C)c(N(c2ccc(N(c3ccccc3)c3cncc(-c4ccccc4)n3)cc2)c2c(C)c([2H])c(C)c([2H])c2C)c1C ; [2H]c1c(C)c([2H])c(C)c(N(c2ccc(N(c3ccccc3)c3cnc(-c4ccccc4)cn3)cc2)c2c(C)c([2H])c(C)c([2H])c2C)c1C]

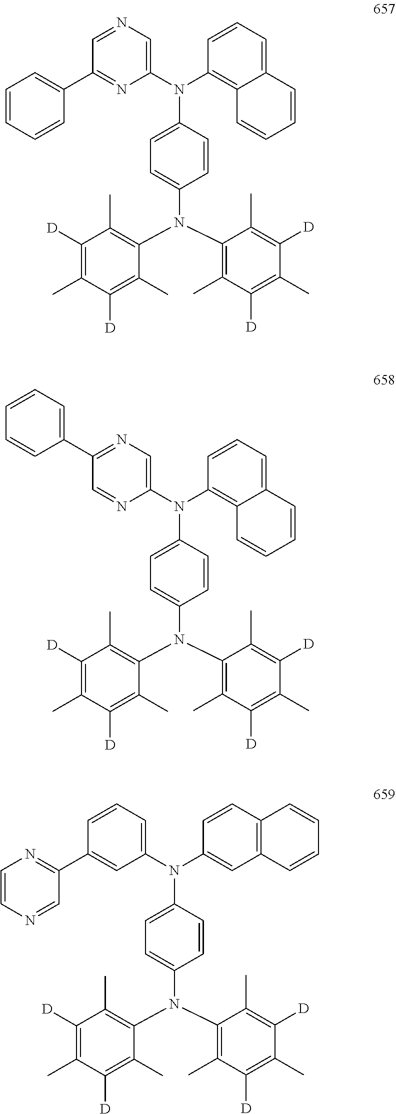 [SMILES: [2H]c1c(C)c([2H])c(C)c(N(c2ccc(N(c3cncc(-c4ccccc4)n3)c3cccc4ccccc34)cc2)c2c(C)c([2H])c(C)c([2H])c2C)c1C ; [2H]c1c(C)c([2H])c(C)c(N(c2ccc(N(c3cnc(-c4ccccc4)cn3)c3cccc4ccccc34)cc2)c2c(C)c([2H])c(C)c([2H])c2C)c1C ; [2H]c1c(C)c([2H])c(C)c(N(c2ccc(N(c3cccc(-c4cnccn4)c3)c3ccc4ccccc4c3)cc2)c2c(C)c([2H])c(C)c([2H])c2C)c1C]

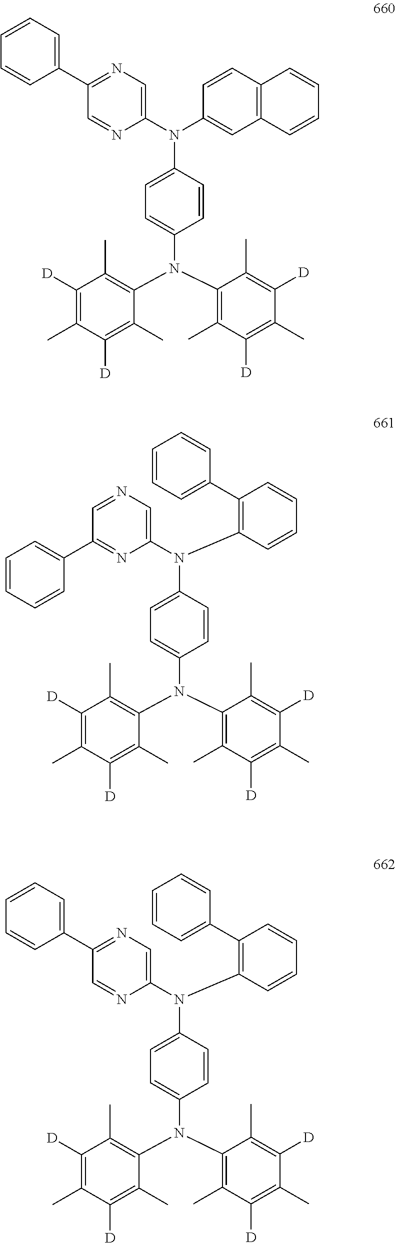 [SMILES: [2H]c1c(C)c([2H])c(C)c(N(c2ccc(N(c3ccc4ccccc4c3)c3cnc(-c4ccccc4)cn3)cc2)c2c(C)c([2H])c(C)c([2H])c2C)c1C ; [2H]c1c(C)c([2H])c(C)c(N(c2ccc(N(c3cncc(-c4ccccc4)n3)c3ccccc3-c3ccccc3)cc2)c2c(C)c([2H])c(C)c([2H])c2C)c1C ; [2H]c1c(C)c([2H])c(C)c(N(c2ccc(N(c3cnc(-c4ccccc4)cn3)c3ccccc3-c3ccccc3)cc2)c2c(C)c([2H])c(C)c([2H])c2C)c1C]

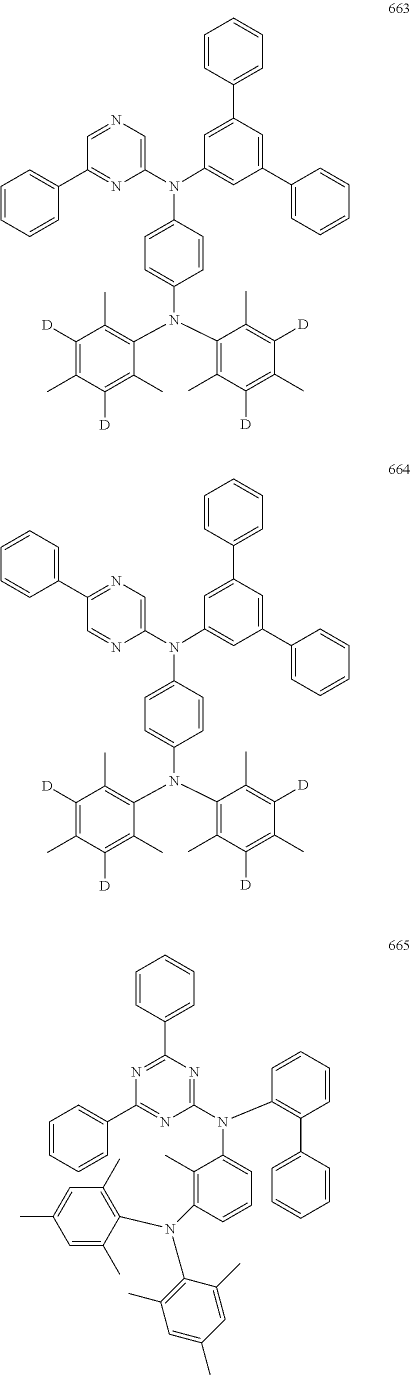 [SMILES: [2H]c1c(C)c([2H])c(C)c(N(c2ccc(N(c3cc(-c4ccccc4)cc(-c4ccccc4)c3)c3cncc(-c4ccccc4)n3)cc2)c2c(C)c([2H])c(C)c([2H])c2C)c1C ; [2H]c1c(C)c([2H])c(C)c(N(c2ccc(N(c3cc(-c4ccccc4)cc(-c4ccccc4)c3)c3cnc(-c4ccccc4)cn3)cc2)c2c(C)c([2H])c(C)c([2H])c2C)c1C ; Cc1cc(C)c(N(c2cccc(N(c3nc(-c4ccccc4)nc(-c4ccccc4)n3)c3ccccc3-c3ccccc3)c2C)c2c(C)cc(C)cc2C)c(C)c1]

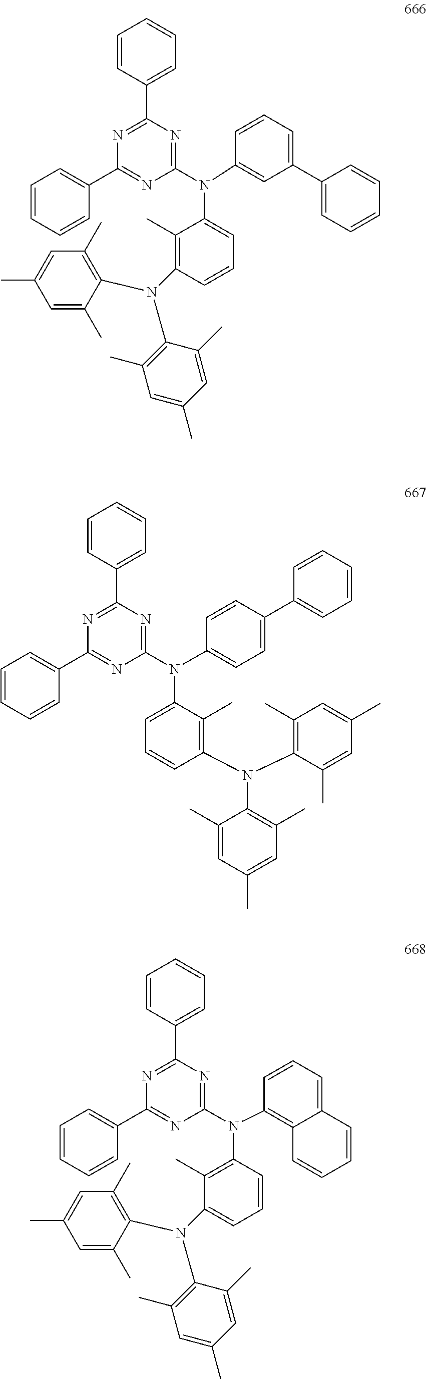

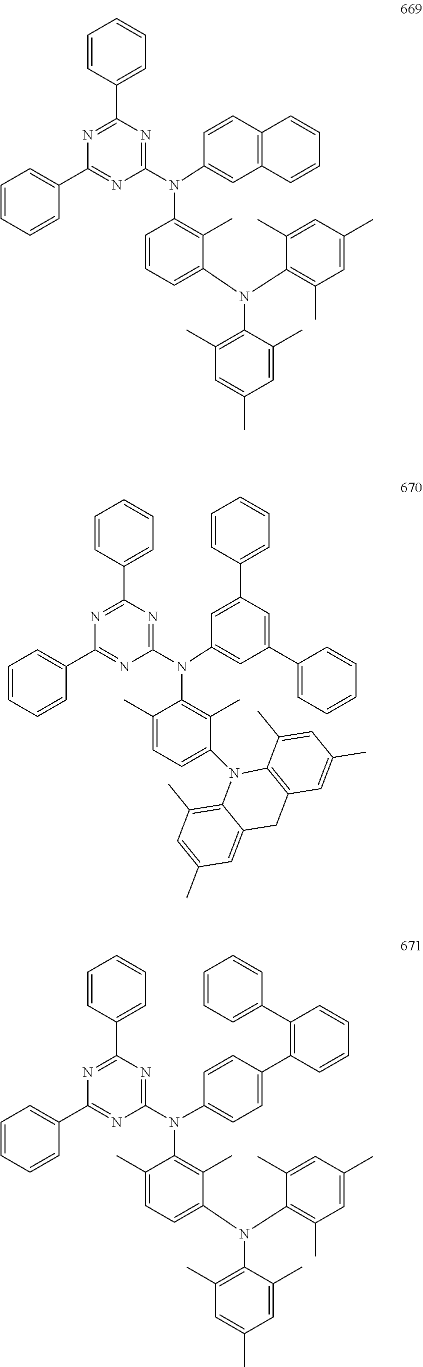 [SMILES: Cc1cc(C)c(N(c2cccc(N(c3ccc4ccccc4c3)c3nc(-c4ccccc4)nc(-c4ccccc4)n3)c2C)c2c(C)cc(C)cc2C)c(C)c1 ; Cc1cc(C)c2c(c1)Cc1cc(C)cc(C)c1N2c1ccc(C)c(N(c2cc(-c3ccccc3)cc(-c3ccccc3)c2)c2nc(-c3ccccc3)nc(-c3ccccc3)n2)c1C ; Cc1cc(C)c(N(c2ccc(C)c(N(c3ccc(-c4ccccc4-c4ccccc4)cc3)c3nc(-c4ccccc4)nc(-c4ccccc4)n3)c2C)c2c(C)cc(C)cc2C)c(C)c1]

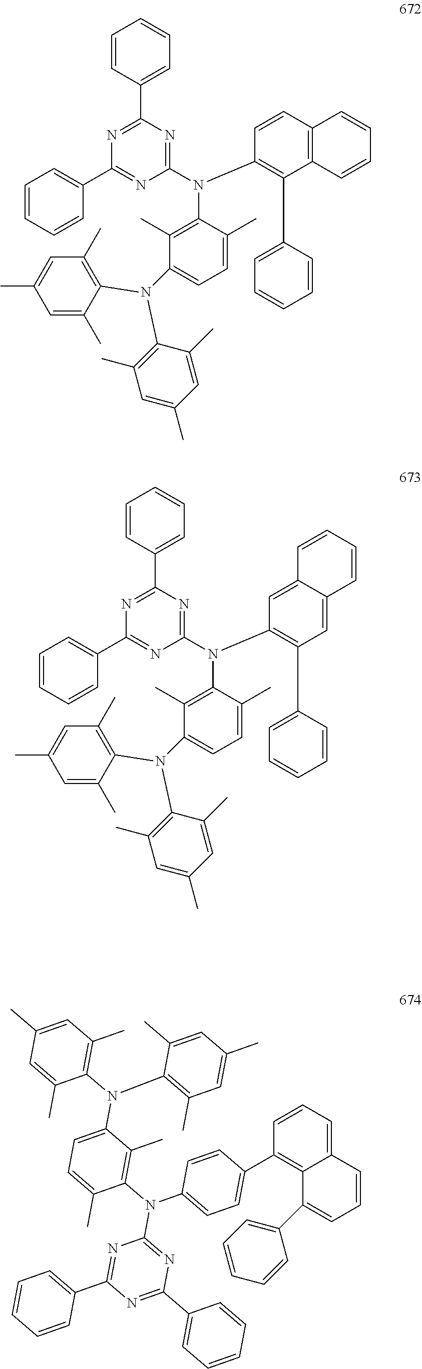 [SMILES: Cc1cc(C)c(N(c2ccc(C)c(N(c3nc(-c4ccccc4)nc(-c4ccccc4)n3)c3ccc4ccccc4c3-c3ccccc3)c2C)c2c(C)cc(C)cc2C)c(C)c1 ; Cc1cc(C)c(N(c2ccc(C)c(N(c3nc(-c4ccccc4)nc(-c4ccccc4)n3)c3cc4ccccc4cc3-c3ccccc3)c2C)c2c(C)cc(C)cc2C)c(C)c1 ; Cc1cc(C)c(N(c2ccc(C)c(N(c3ccc(-c4cccc5cccc(-c6ccccc6)c45)cc3)c3nc(-c4ccccc4)nc(-c4ccccc4)n3)c2C)c2c(C)cc(C)cc2C)c(C)c1]

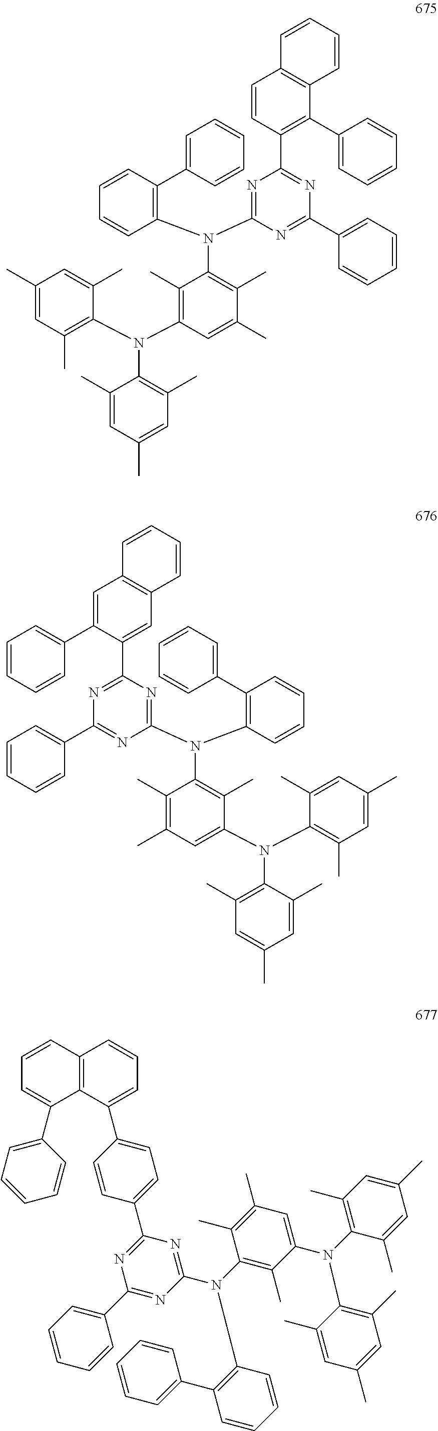 [SMILES: Cc1cc(C)c(N(c2cc(C)c(C)c(N(c3nc(-c4ccccc4)nc(-c4ccc5ccccc5c4-c4ccccc4)n3)c3ccccc3-c3ccccc3)c2C)c2c(C)cc(C)cc2C)c(C)c1 ; Cc1cc(C)c(N(c2cc(C)c(C)c(N(c3nc(-c4ccccc4)nc(-c4cc5ccccc5cc4-c4ccccc4)n3)c3ccccc3-c3ccccc3)c2C)c2c(C)cc(C)cc2C)c(C)c1 ; Cc1cc(C)c(N(c2cc(C)c(C)c(N(c3nc(-c4ccccc4)nc(-c4ccc(-c5cccc6cccc(-c7ccccc7)c56)cc4)n3)c3ccccc3-c3ccccc3)c2C)c2c(C)cc(C)cc2C)c(C)c1]

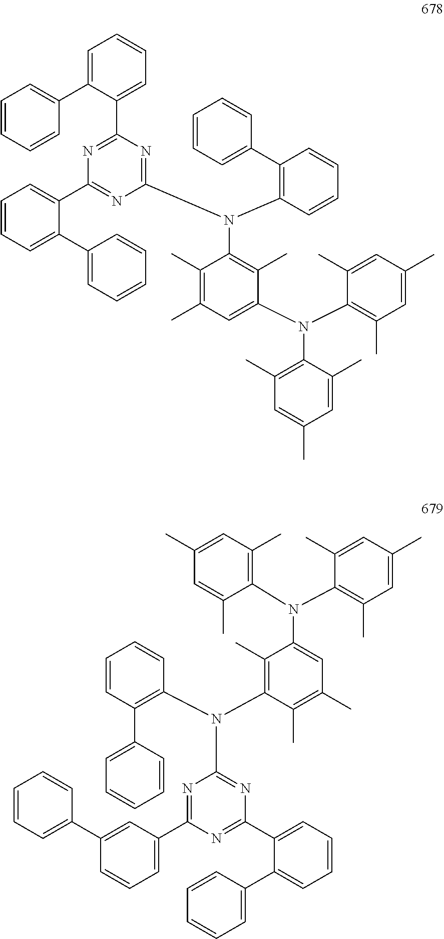 [SMILES: Cc1cc(C)c(N(c2cc(C)c(C)c(N(c3nc(-c4ccccc4-c4ccccc4)nc(-c4ccccc4-c4ccccc4)n3)c3ccccc3-c3ccccc3)c2C)c2c(C)cc(C)cc2C)c(C)c1 ; Cc1cc(C)c(N(c2cc(C)c(C)c(N(c3nc(-c4cccc(-c5ccccc5)c4)nc(-c4ccccc4-c4ccccc4)n3)c3ccccc3-c3ccccc3)c2C)c2c(C)cc(C)cc2C)c(C)c1]

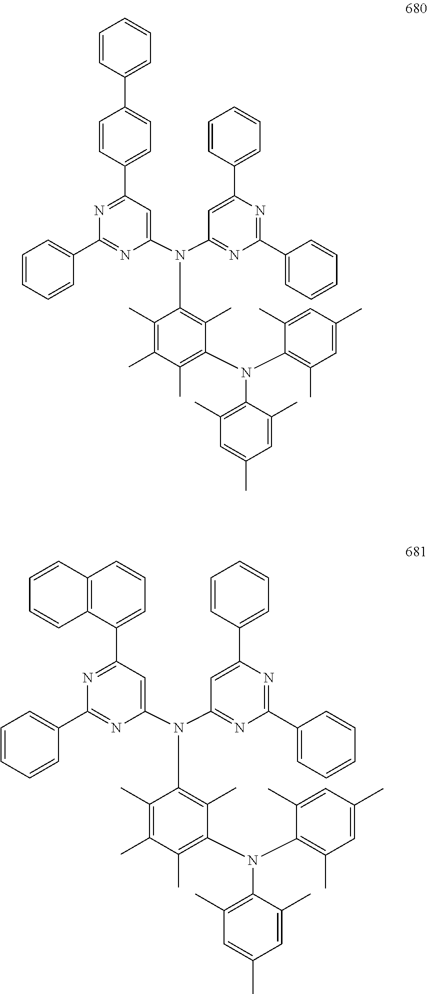 [SMILES: Cc1cc(C)c(N(c2c(C)cc(C)cc2C)c2c(C)c(C)c(C)c(N(c3cc(-c4ccccc4)nc(-c4ccccc4)n3)c3cc(-c4ccc(-c5ccccc5)cc4)nc(-c4ccccc4)n3)c2C)c(C)c1 ; Cc1cc(C)c(N(c2c(C)cc(C)cc2C)c2c(C)c(C)c(C)c(N(c3cc(-c4ccccc4)nc(-c4ccccc4)n3)c3cc(-c4cccc5ccccc45)nc(-c4ccccc4)n3)c2C)c(C)c1]

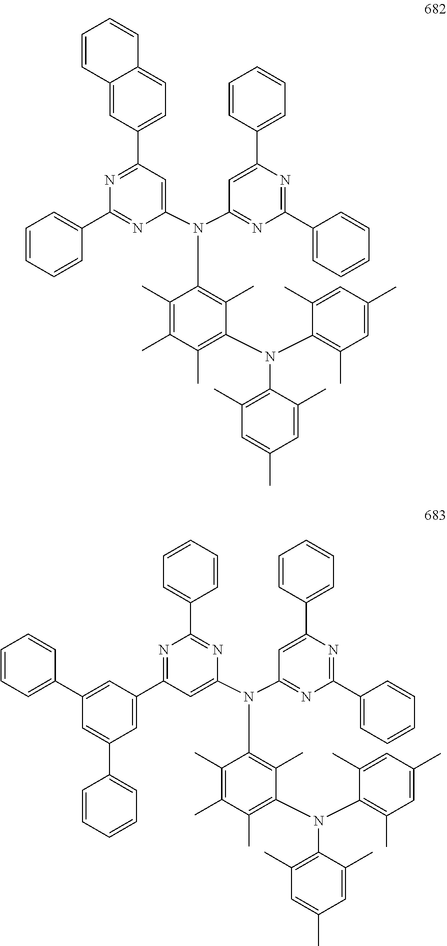 [SMILES: Cc1cc(C)c(N(c2c(C)cc(C)cc2C)c2c(C)c(C)c(C)c(N(c3cc(-c4ccccc4)nc(-c4ccccc4)n3)c3cc(-c4ccc5ccccc5c4)nc(-c4ccccc4)n3)c2C)c(C)c1 ; Cc1cc(C)c(N(c2c(C)cc(C)cc2C)c2c(C)c(C)c(C)c(N(c3cc(-c4ccccc4)nc(-c4ccccc4)n3)c3cc(-c4cc(-c5ccccc5)cc(-c5ccccc5)c4)nc(-c4ccccc4)n3)c2C)c(C)c1]

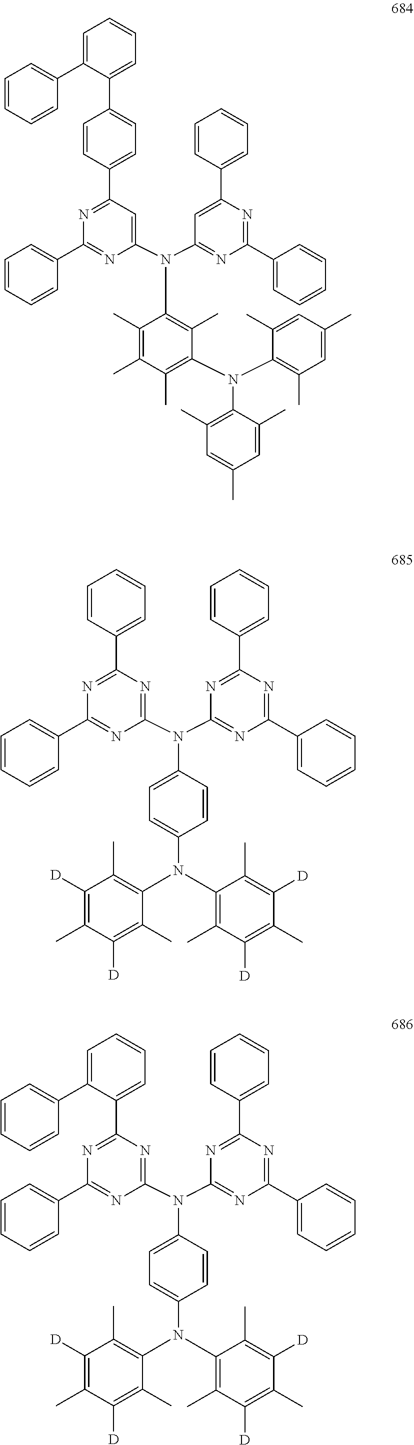 [SMILES: Cc1cc(C)c(N(c2c(C)cc(C)cc2C)c2c(C)c(C)c(C)c(N(c3cc(-c4ccccc4)nc(-c4ccccc4)n3)c3cc(-c4ccc(-c5ccccc5-c5ccccc5)cc4)nc(-c4ccccc4)n3)c2C)c(C)c1 ; [2H]c1c(C)c([2H])c(C)c(N(c2ccc(N(c3nc(-c4ccccc4)nc(-c4ccccc4)n3)c3nc(-c4ccccc4)nc(-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)c([2H])c(C)c([2H])c2C)c1C]

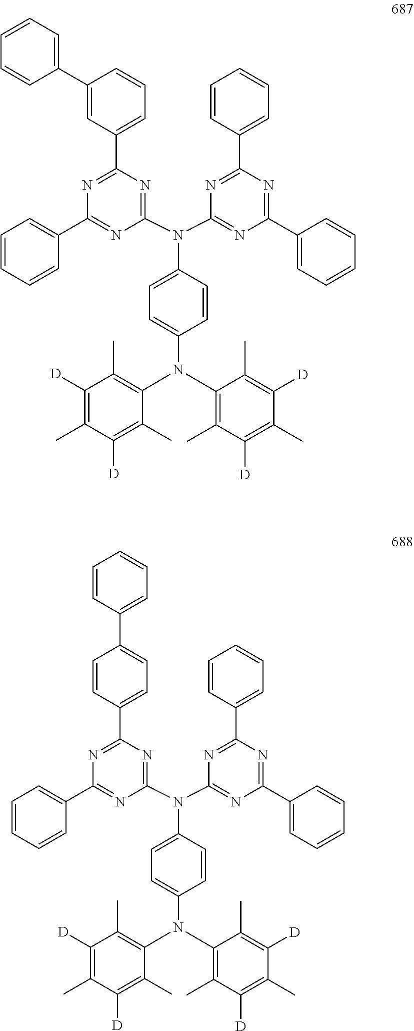 [SMILES: [2H]c1c(C)c([2H])c(C)c(N(c2ccc(N(c3nc(-c4ccccc4)nc(-c4ccccc4)n3)c3nc(-c4ccccc4)nc(-c4cccc(-c5ccccc5)c4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4ccc(-c5ccccc5)cc4)n3)cc2)c2c(C)c([2H])c(C)c([2H])c2C)c1C]

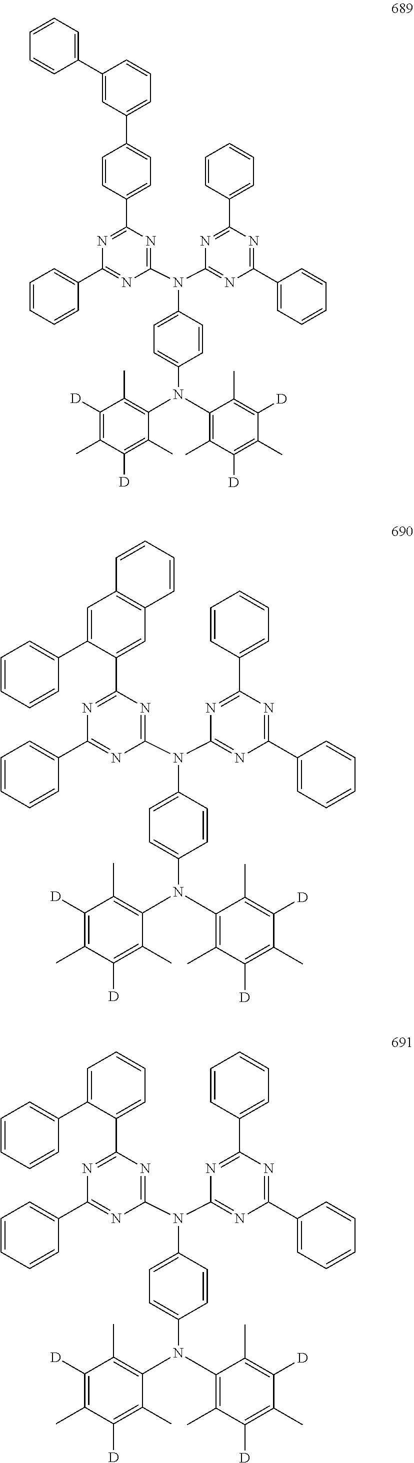 [SMILES: [2H]c1c(C)c([2H])c(C)c(N(c2ccc(N(c3nc(-c4ccccc4)nc(-c4ccccc4)n3)c3nc(-c4ccccc4)nc(-c4ccc(-c5cccc(-c6ccccc6)c5)cc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4cc5ccccc5cc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)c([2H])c(C)c([2H])c2C)c1C]

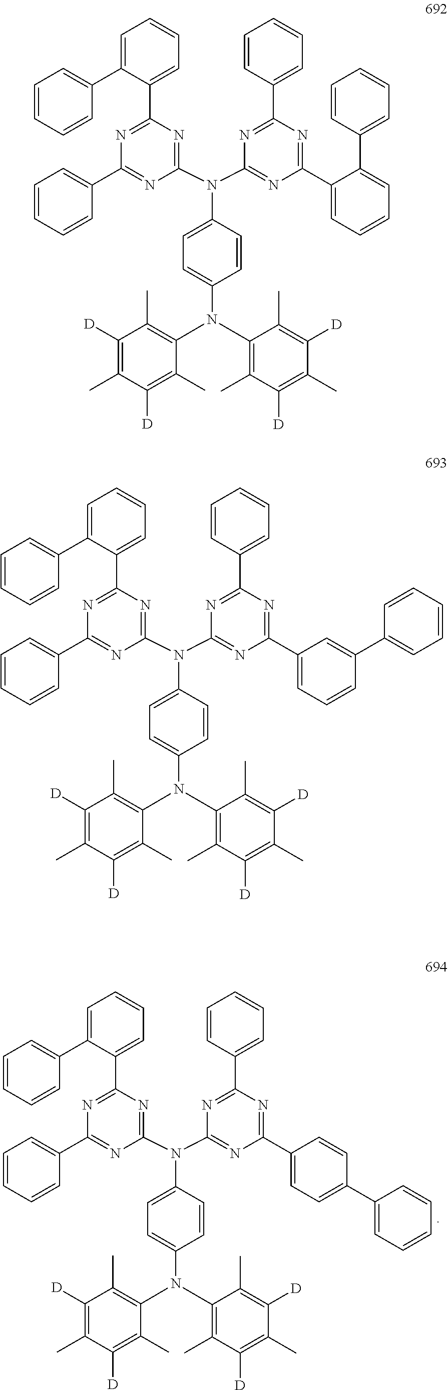 [SMILES: [2H]c1c(C)c([2H])c(C)c(N(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)c([2H])c(C)c([2H])c2C)c1C]

The amine-containing compound may be a compound having a structure represented by Formula 1 and may include two or more benzene moieties bonded to heteroatom X. In one or more embodiments, the amine-containing compound may include a group represented by Formula 2 bonded to the nitrogen of the amine group, wherein in Formula 2, Y₁ may be C(R₁₁) or N, Y₂ may be C(R₁₂) or N, Y₃ may be C(R₁₃) or N, Y₄ may be C(R₁₄) or N, Y₅ may be C(R₁₅) or N, and at least two selected from among Y₁ to Y₅ may each be N.

Accordingly, the amine-containing compound may improve color purity and driving voltage of the light-emitting device. Moreover, in the light-emitting device employing the amine-containing compound, energy may be easily transferred within the light-emitting device, resulting in the improvement of lifespan characteristics.

Synthesis methods of the amine-containing compound represented by Formula 1 may be recognizable by one of ordinary skill in the art by referring to Synthesis Examples and/or Examples provided herein.

At least one amine-containing compound represented by Formula 1 may be utilized in a light-emitting device (for example, an organic light-emitting device). Thus, one or more aspects of the disclosure is directed toward a light-emitting device including: a first electrode; a second electrode facing the first electrode; an interlayer between the first electrode and the second electrode and including an emission layer; and the amine-containing compound represented by Formula 1 as described herein.

In some embodiments, the first electrode of the light-emitting device may be an anode,

the second electrode of the light-emitting device may be a cathode,

the interlayer may further include a hole transport region between the first electrode and the emission layer and an electron transport region between the emission layer and the second electrode,

the hole transport region may include a hole injection layer, a hole transport layer, an emission auxiliary layer, an electron blocking layer, or any combination thereof, and

the electron transport region may include a hole blocking layer, an electron transport layer, an electron injection layer, an electron control layer, or any combination thereof.

In one or more embodiments, the amine-containing compound represented by Formula 1 may be included in the interlayer.

In one or more embodiments, the amine-containing compound represented by Formula 1 may be included in the electron transport region.

In one or more embodiments, the amine-containing compound represented by Formula 1 may be included in the electron transport layer.

In one or more embodiments, the amine-containing compound may be included in the electron transport layer, and the electron transport layer and the emission layer may be in direct contact with each other.

In one or more embodiments, the light-emitting device may further include a capping layer arranged outside of (e.g., on) the second electrode, and the amine-containing compound may be included in the capping layer.

In one or more embodiments, the light-emitting device may further include:

A first capping layer located outside (e.g., on) the first electrode; and/or

a second capping layer located outside (e.g., on) the second electrode,

wherein the amine-containing compound may be included in the first capping layer and/or the second capping layer.

In one or more embodiments, the emission layer may be to emit blue light.

In one or more embodiments, the dopant may be a phosphorescent dopant or a delayed fluorescence dopant.

In one or more embodiments, the host may include a first host including at least one electron donating group and a second host including at least one electron withdrawing group.

In one or more embodiments, the emission layer further include a sensitizer.

In one or more embodiments, the emission layer may include a delayed fluorescence material.

In one or more embodiments, the emission layer may be to emit blue light.

In one or more embodiments, the emission layer may further include a first host and a second host, wherein the first host may be a hole-transporting compound including at least one electron-donating group, and the second host may be an electron-transporting host including at least one electron-withdrawing group.

In one or more embodiments, the emission layer may further include a third compound, and the third compound may be a metal-containing compound.

In one or more embodiments, the third compound may serve as a sensitizer such as a phosphorescent sensitizer.

In one or more embodiments, the third compound may not emit light.

In some embodiments, the emission layer may further include at least one of an auxiliary dopant or a sensitizer.

In one or more embodiments, the auxiliary dopant and the sensitizer may each independently be an organometallic compound including Pt and a tetradentate ligand bonded to Pt, wherein the tetradentate ligand may include a carbene moiety chemically bonded to the Pt. For example, the auxiliary dopant and/or the sensitizer may include the third compound.

In one or more embodiments, the first host and the second host may serve as an exciplex host.

The term “electron-donating group” refers to any moiety having ability to donate electrons, and for example, may be a π electron-rich C₃-C₆₀ cyclic group or an amine group, but embodiments of the present disclosure are not limited thereto. The electron-donating group may refer to a cyclic group other than a π electron-deficient nitrogen-containing C₁-C₆₀ cyclic group.

The term “electron-withdrawing group” refers to any moiety having ability to withdraw electrons, and for example, may be —F, —CFH₂, —CF₂H, —CF₃, —CN, —NO₂, a π electron-deficient nitrogen-containing C₁-C₆₀ cyclic group, or any combination thereof. However, embodiments of the present disclosure are not limited thereto.

Regarding a luminescence pathway in the light-emitting device according to one or more embodiments, the first host and the second host may form an exciton (1st step), the energy of the exciton may be transferred to the third compound (2nd step), and the energy may be transferred from the third compound to the organometallic compound (3rd step).

In one or more embodiments, an amount of the third compound may be more than 0 parts by weight and less than 50 parts by weight based on the total weight of 100 parts by weight of the emission layer.

In one or more embodiments, the first host may include at least one carbazole moiety, and the second host may include at least one azine moiety.

In one or more embodiments, the first host may be represented by Formula 301-1A or 301-2A:

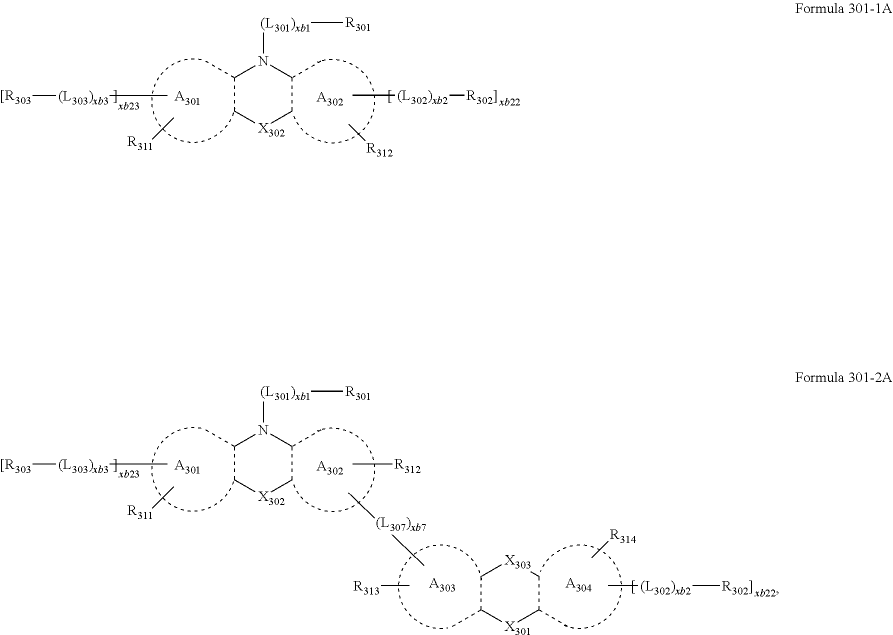

wherein, in Formulae 301-1A and 301-2A,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄ₐ], C(R₃₀₄ₐ)(R₃₀₄ₐ), or Si(R₃₀₄ₐ)(R₃₀₅),
X₃₀₂ may be a single bond, O, S, N[(L₃₀₅)_{xb5}-R₃₀₅ₐ], C(R₃₀₅ₐ)(R_{305b}), or Si(R₃₀₅ₐ)(R_{305b}),

X₃₀₃ may be a single bond, O, S, N[(L₃₀₆)_{xb6}-R₃₀₆ₐ], C(R₃₀₆ₐ)(R_{306b}), or Si(R₃₀₆ₐ)(R_{306b}),

xb22 and xb23 may each independently be an integer from 0 to 10,

L₃₀₁ to L₃₀₇ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb1 to xb7 may each independently be an integer from 0 to 5,

R₃₀₁ to R₃₀₃, R₃₀₄ₐ to R₃₀₆ₐ, R_{304b} to R_{306b}, and R₃₁₁ to R₃₁₄ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), and

Q₃₀₁ to Q₃₀₃ are each the same as described with respect to Q₁.

In one or more embodiments, the first host may be one of (e.g., one selected from among) Compounds HTH1 to HTH56, but embodiments of the present disclosure are not limited thereto:

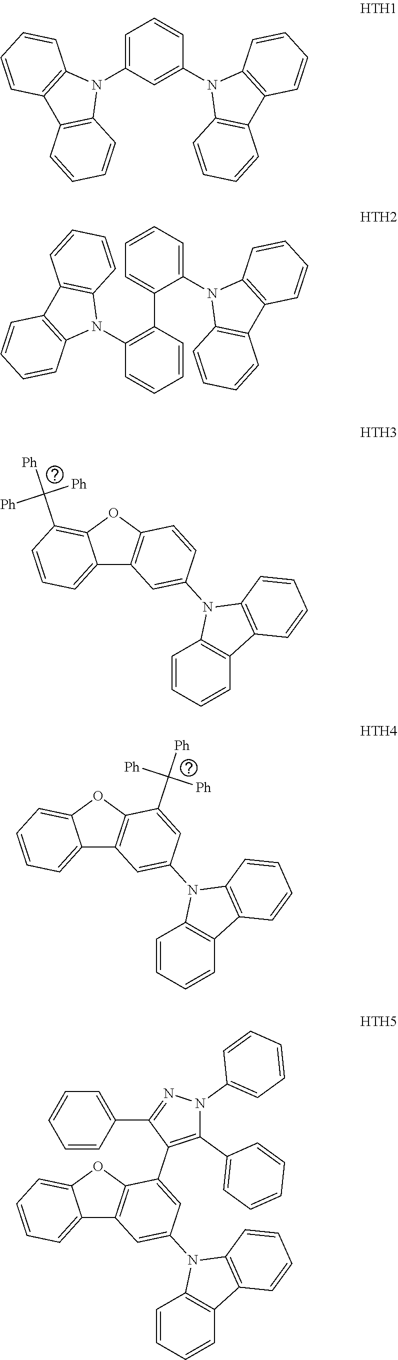

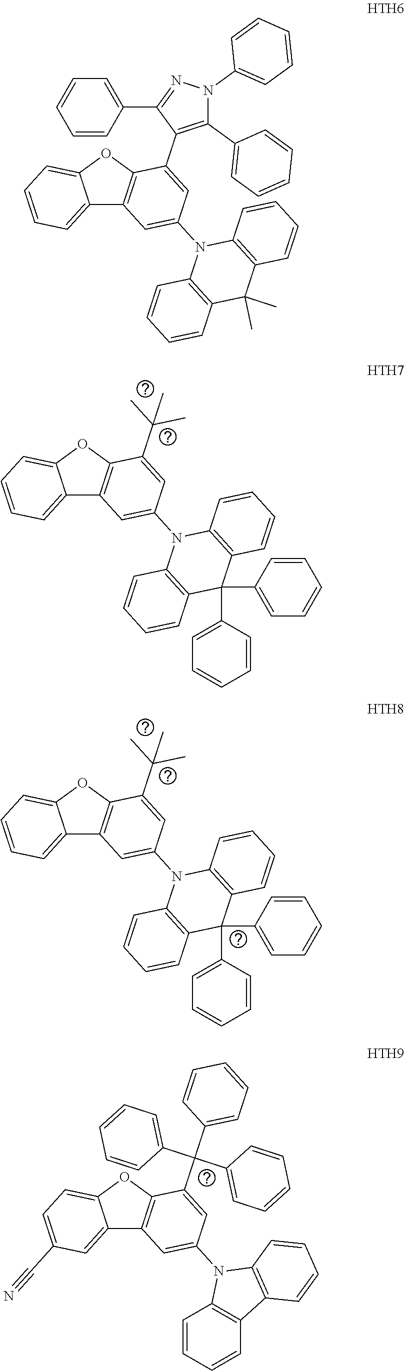

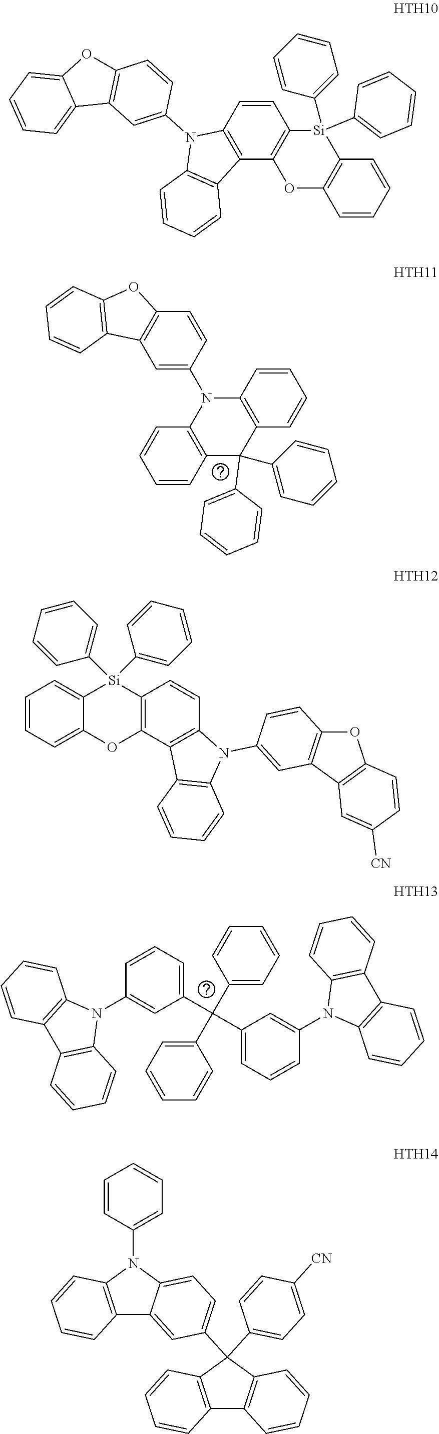 [SMILES: c1ccc([Si]2(c3ccccc3)c3ccccc3Oc3c2ccc2c3c3ccccc3n2-c2ccc3oc4ccccc4c3c2)cc1 ; c1ccc(C2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc(C(c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

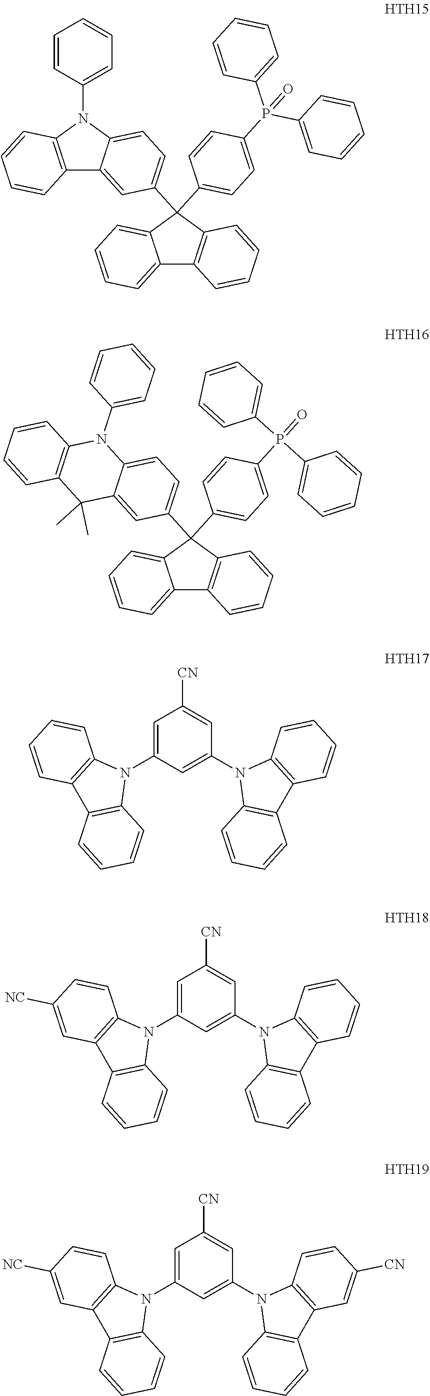 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1]

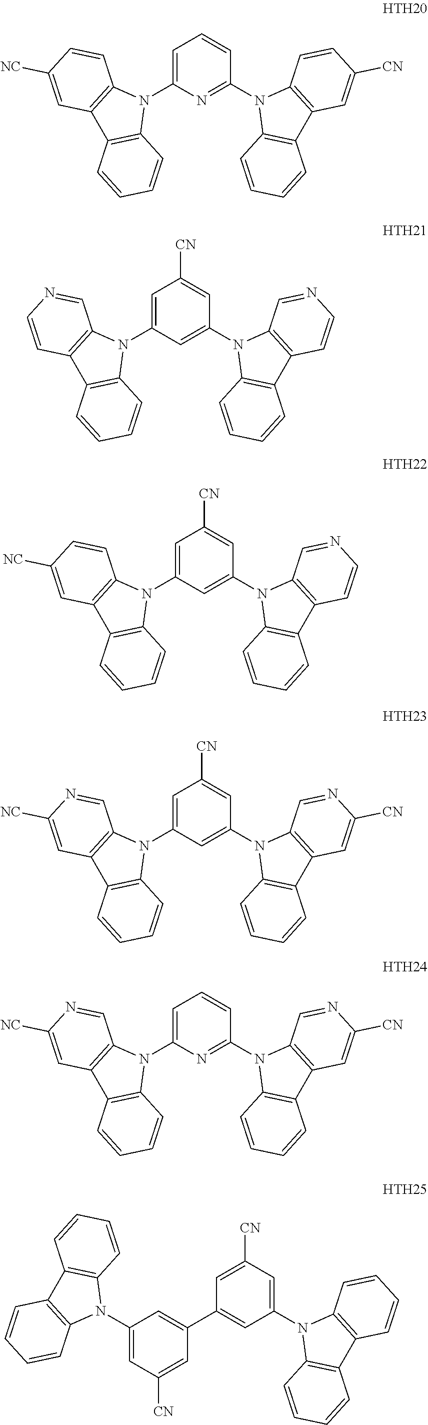 [SMILES: N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1]

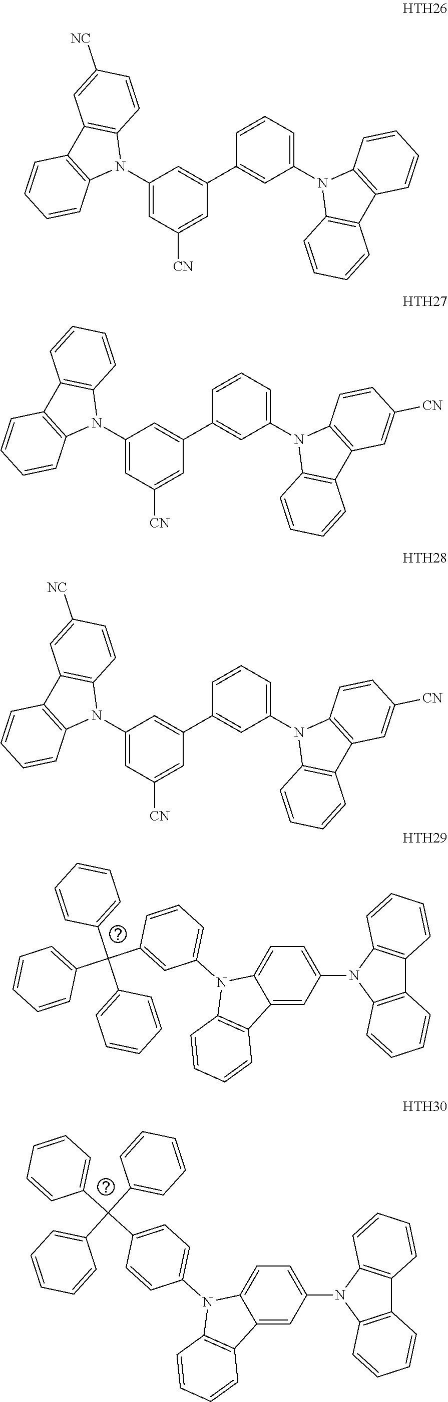 [SMILES: N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc(C(c2ccccc2)(c2ccccc2)c2cccc(-n3c4ccccc4c4cc(-n5c6ccccc6c6ccccc65)ccc43)c2)cc1 ; c1ccc(C(c2ccccc2)(c2ccccc2)c2ccc(-n3c4ccccc4c4cc(-n5c6ccccc6c6ccccc65)ccc43)cc2)cc1]

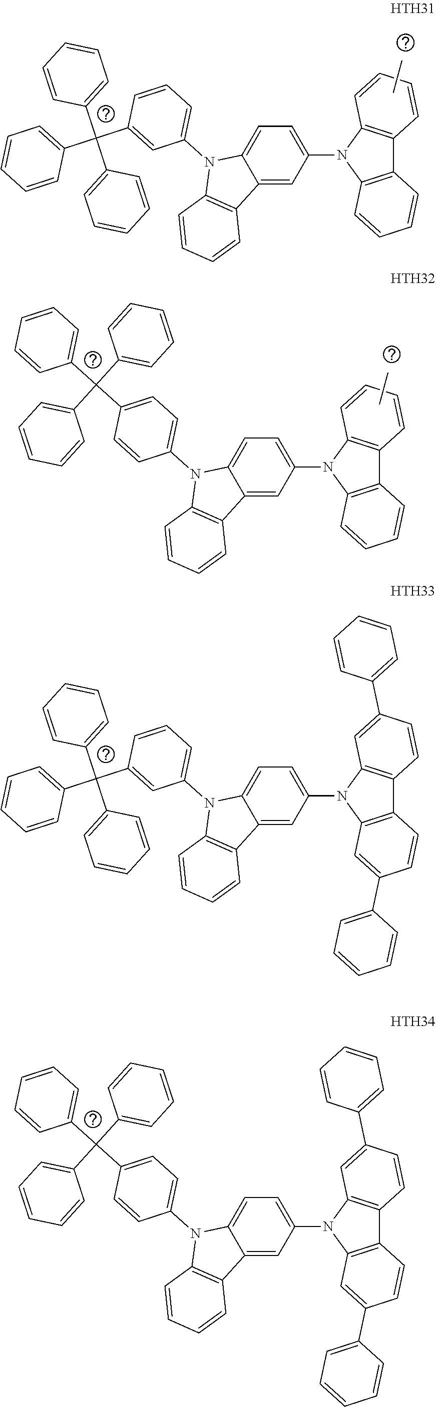

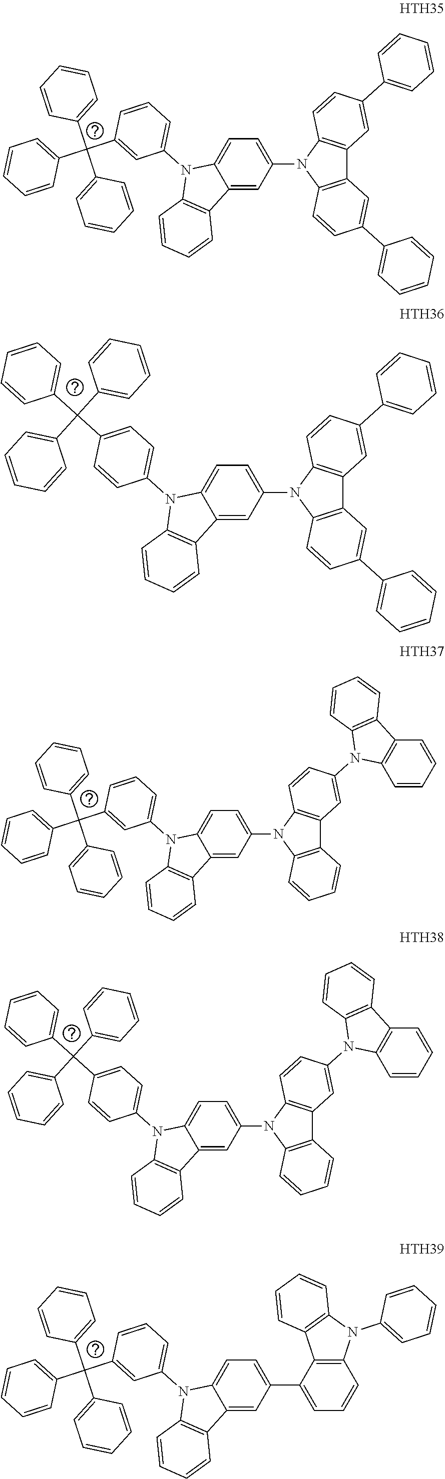

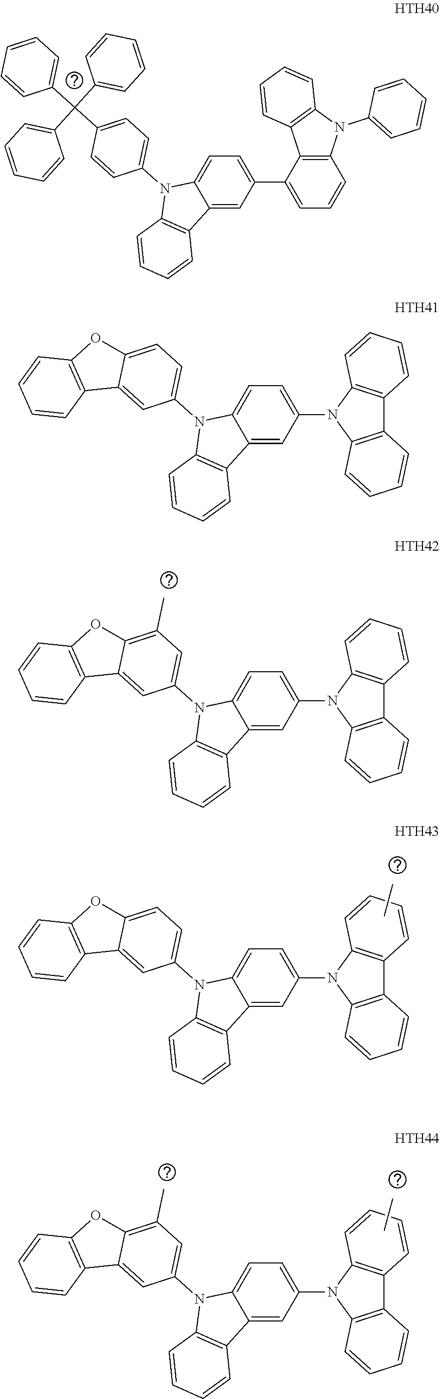

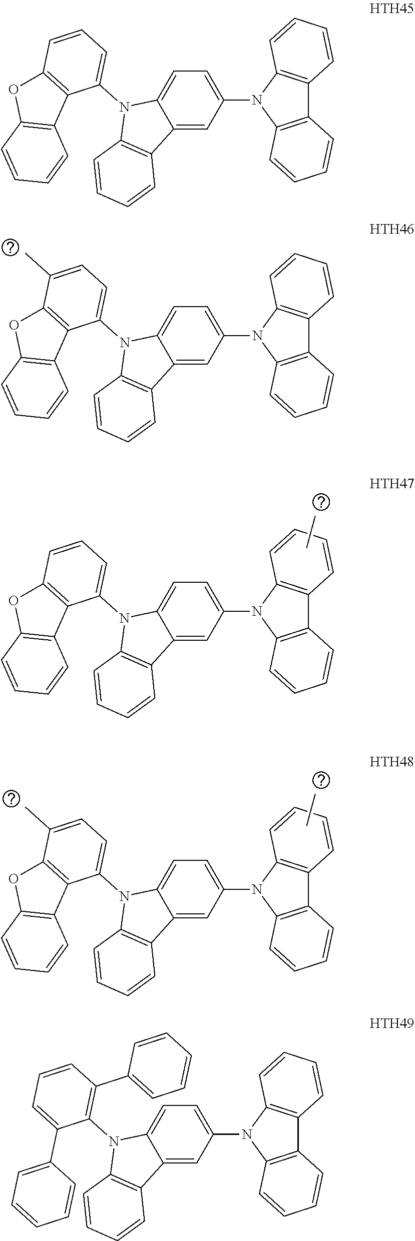

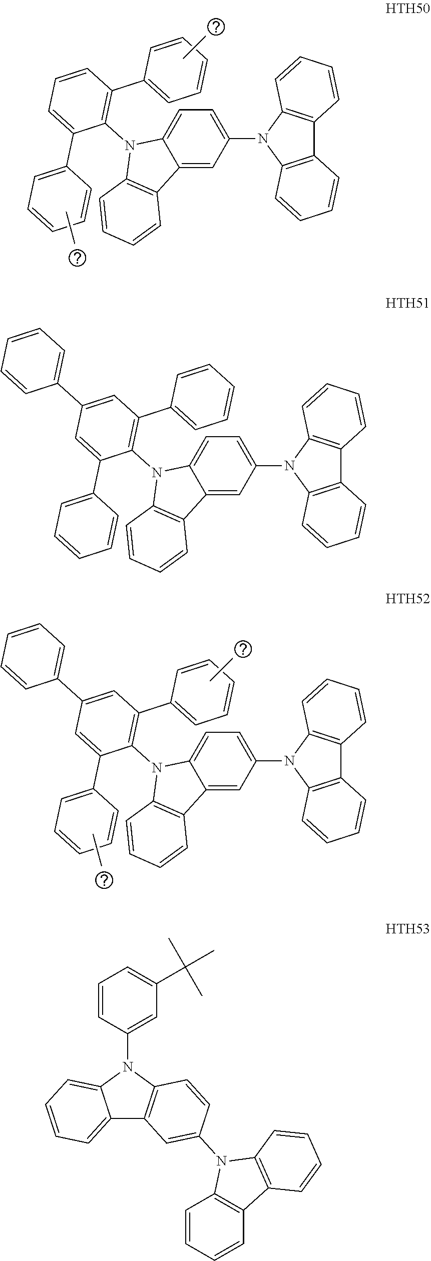

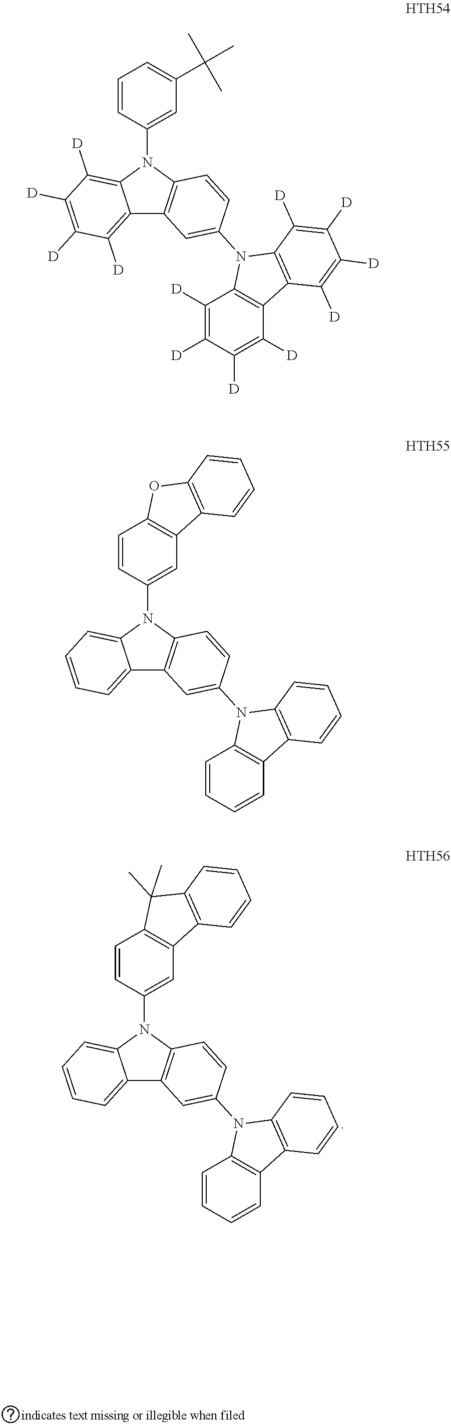 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12 ; CC1(C)c2ccccc2-c2cc(-n3c4ccccc4c4cc(-n5c6ccccc6c6ccccc65)ccc43)ccc21]

In one or more embodiments, the second host may be represented by Formula 302:

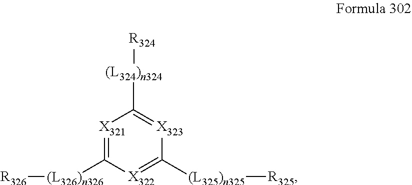

wherein, in Formula 302,

X₃₂₁ may be C(R₃₂₁) or N,

X₃₂₂ may be C(R₃₂₂) or N,

X₃₂₃ may be C(R₃₂₃) or N,

at least one selected from among X₃₂₁ to X₃₂₃ may be N,

L₃₂₄ to L₃₂₆ may each independently be a single bond, a C₃-C₆₀ carbocyclic group unsubstituted or substituted with at least one R₁₀ₐ, C₁-C₆₀ heterocyclic group unsubstituted or substituted with at least one R₁₀ₐ, *—C(Q₃₂₁)(Q₃₂₂)-*′, *—Si(Q₃₂₁)(Q₃₂₂)-*′, *—B(Q₃₂₁)-*′, or *—N(Q₃₂₁)-*′,

n324 to n326 may each independently be an integer from 1 to 5,

R₃₂₁ to R₃₂₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₂₃)(Q₃₂₄)(Q₃₂₅), —N(Q₃₂₃)(Q₃₂₄), —B(Q₃₂₃)(Q₃₂₄), —C(═O)(Q₃₂₃), —S(═O)₂(Q₃₂₃), or —P(═O)(Q₃₂₃)(Q₃₂₄),

two or more neighboring groups among Q₃₂₁ to Q₃₂₅ and R₃₂₁ to R₃₂₆ may optionally be bonded to each other to form a C₅-C₃₀ carbocyclic group unsubstituted or substituted with at least one R₁₀ₐ or a C₂-C₃₀ heterocyclic group unsubstituted or substituted with at least one R₁₀ₐ,

* and *′ each indicate a binding site to a neighboring atom,

R₁₀ₐ is the same as described herein, and

Q₃₂₁ to Q₃₂₆ are each the same as described with respect to Q₁.

In one or more embodiments, the second host may be one of (e.g., one selected from among) Compounds ETH1 to ETH86, but embodiments of the present disclosure are not limited thereto:

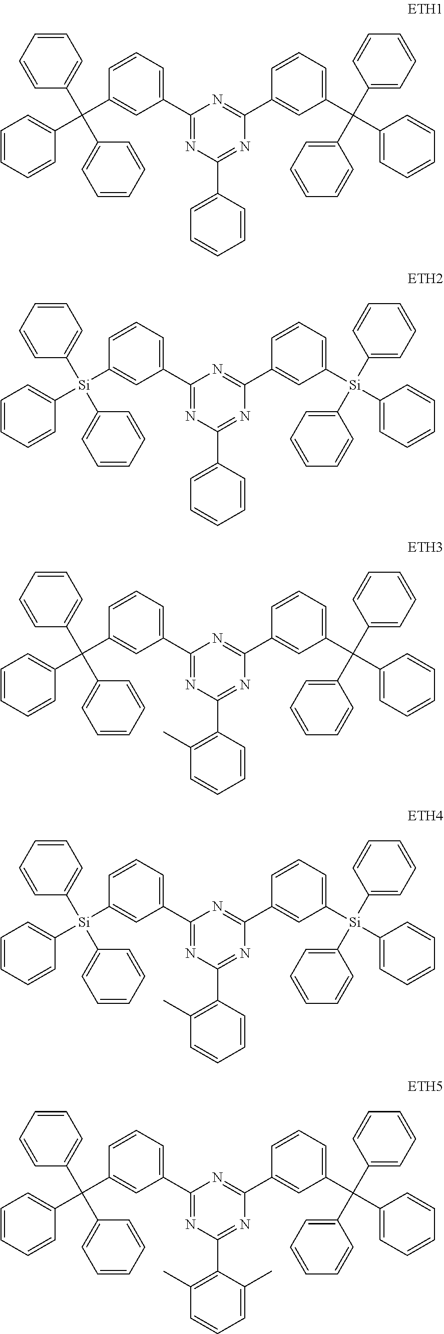

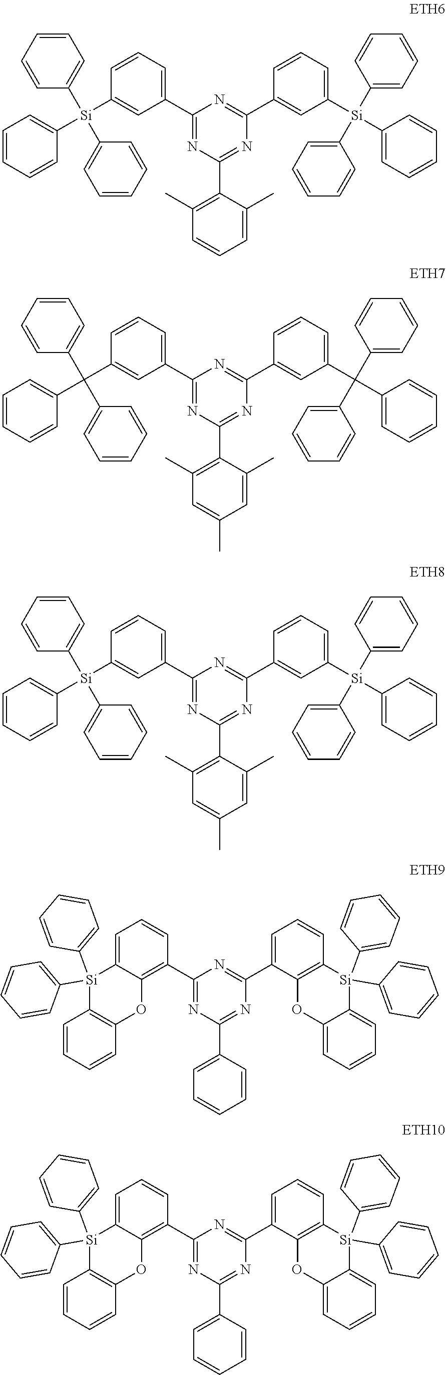

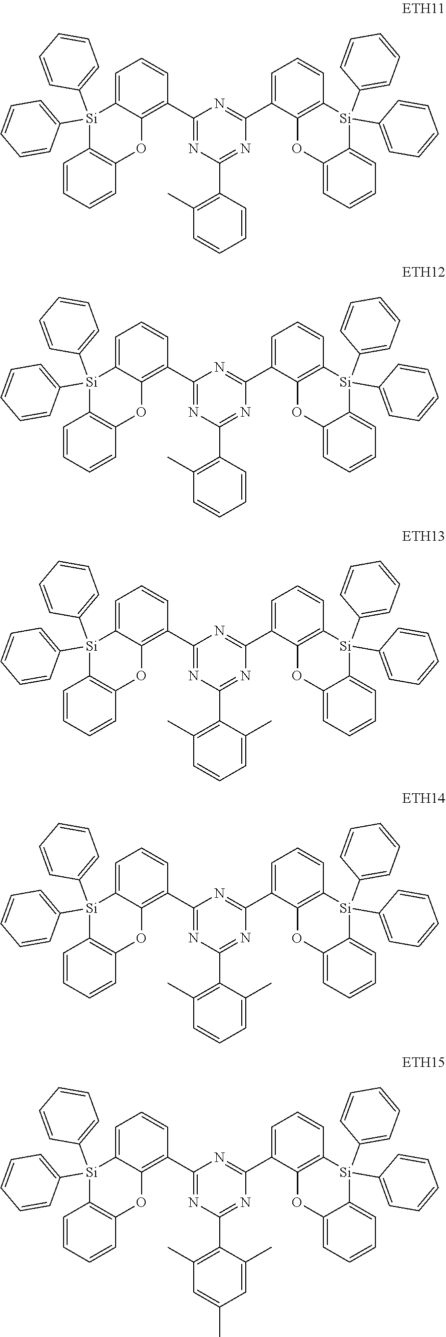

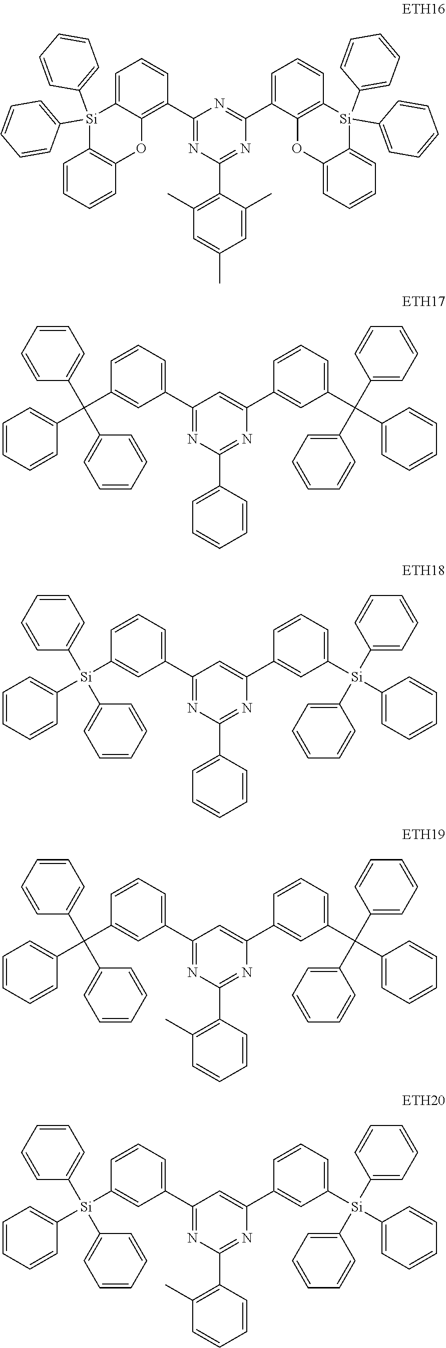

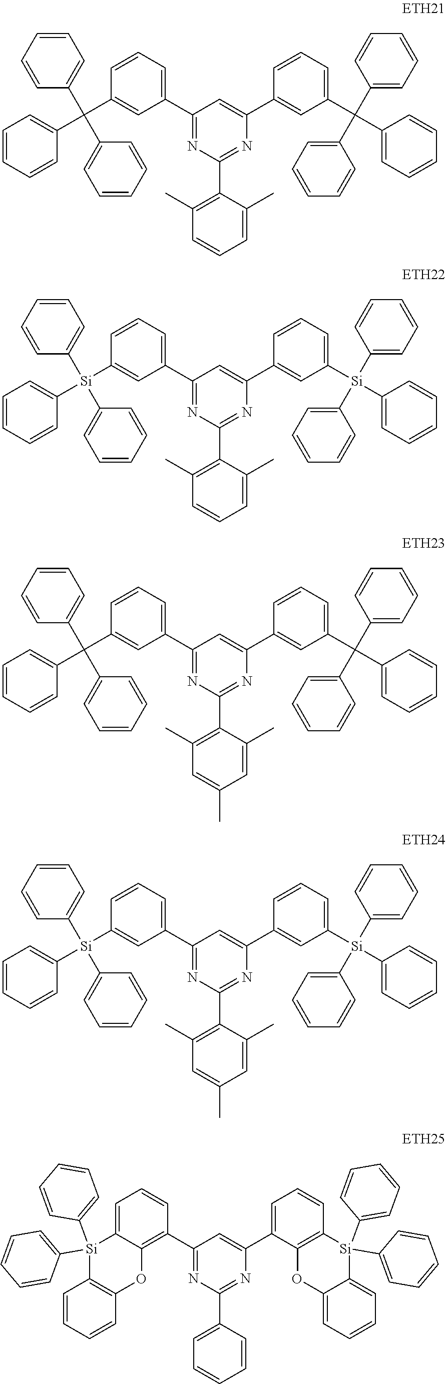

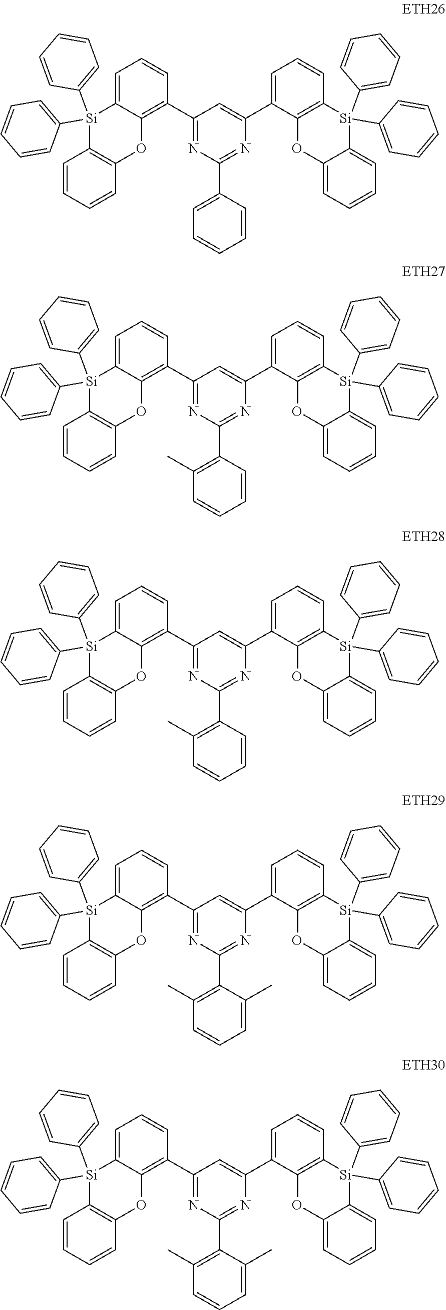

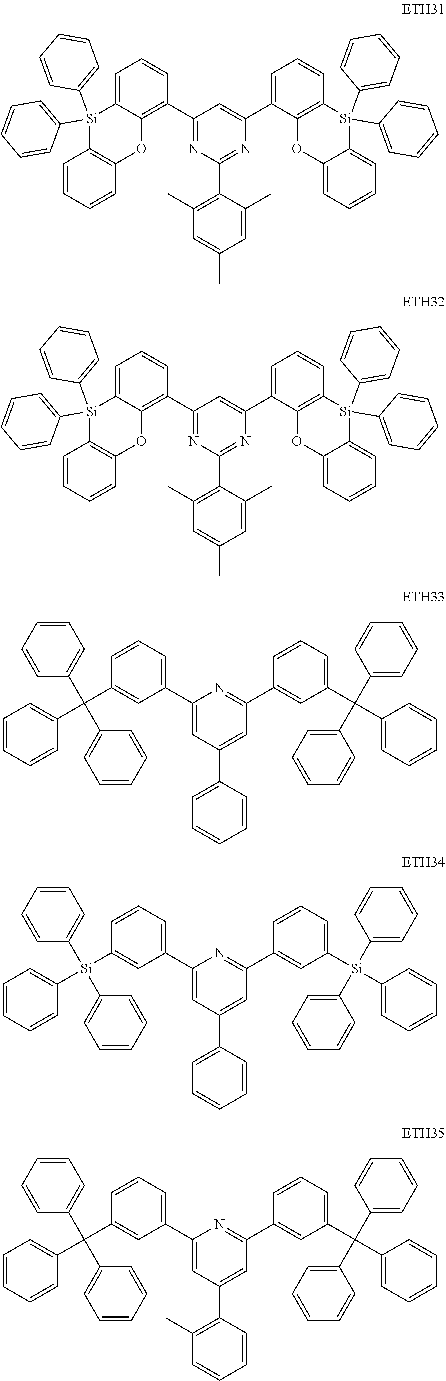

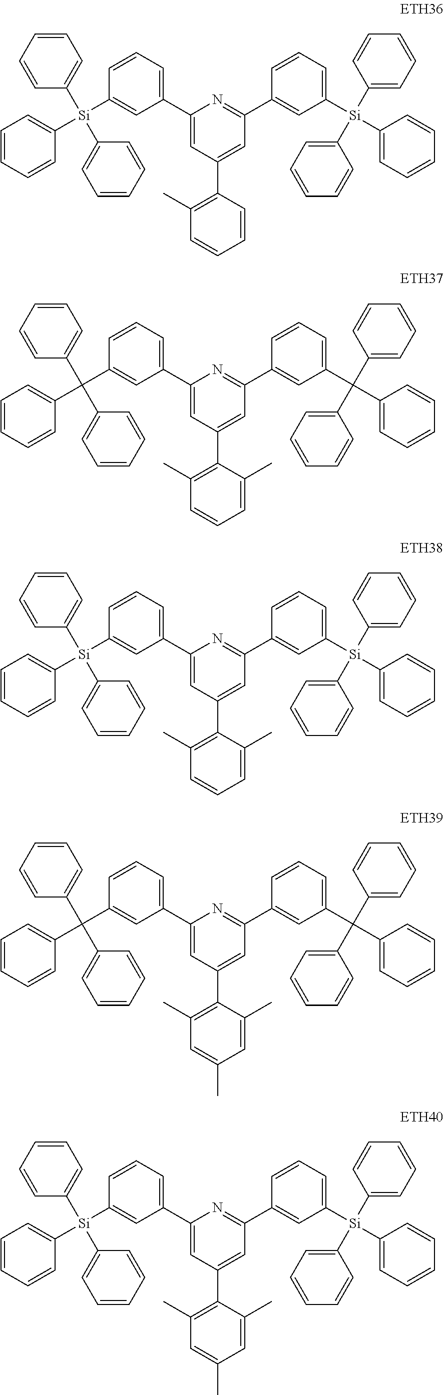

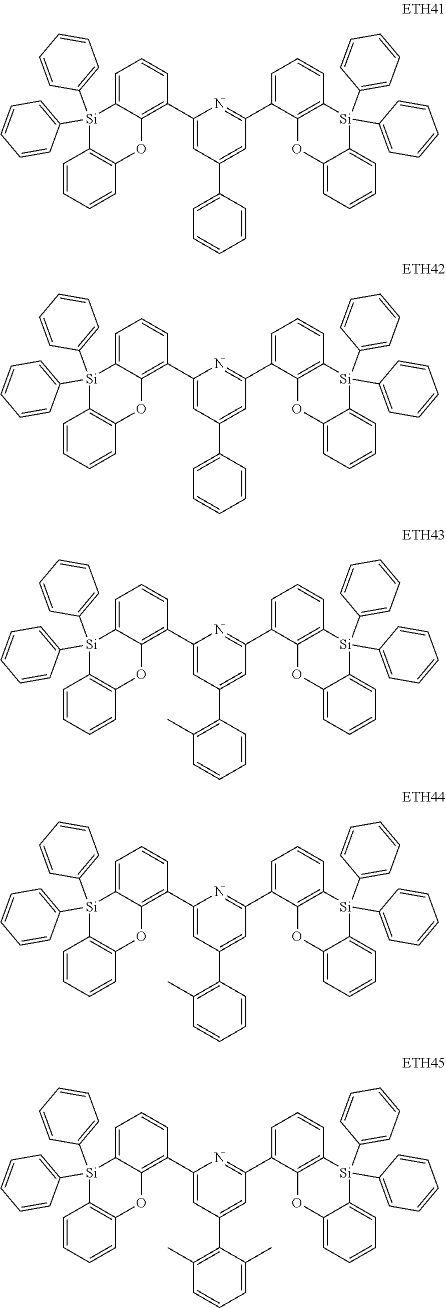

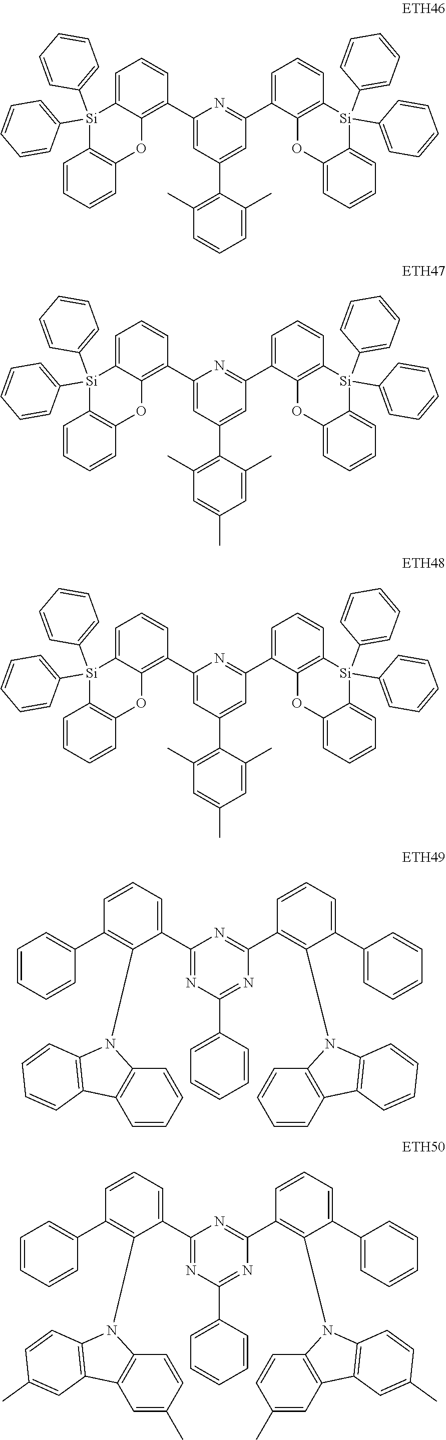

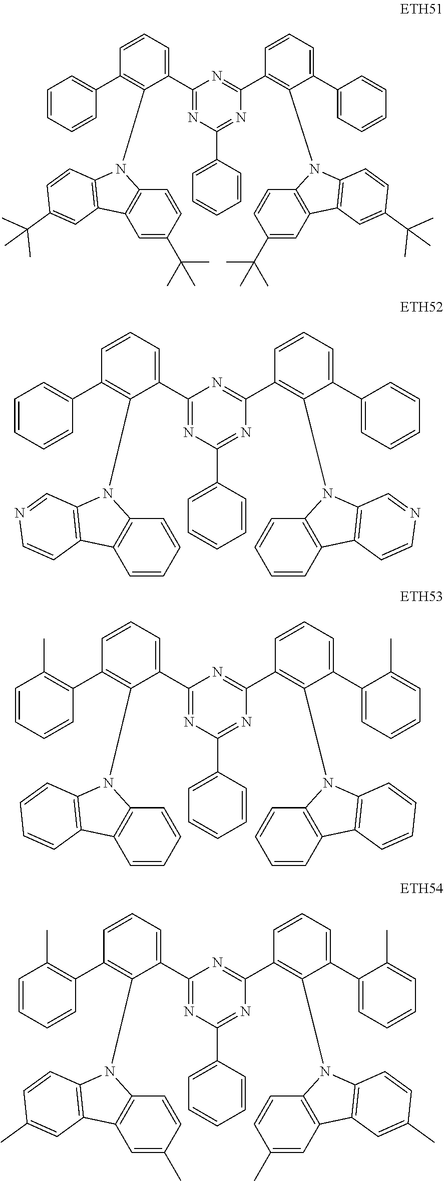

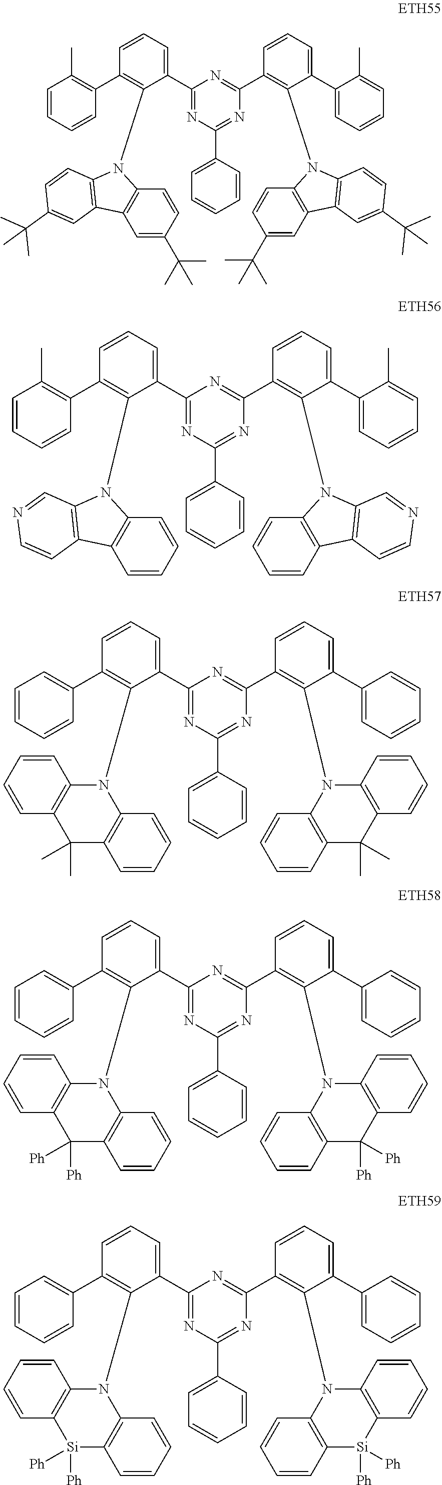

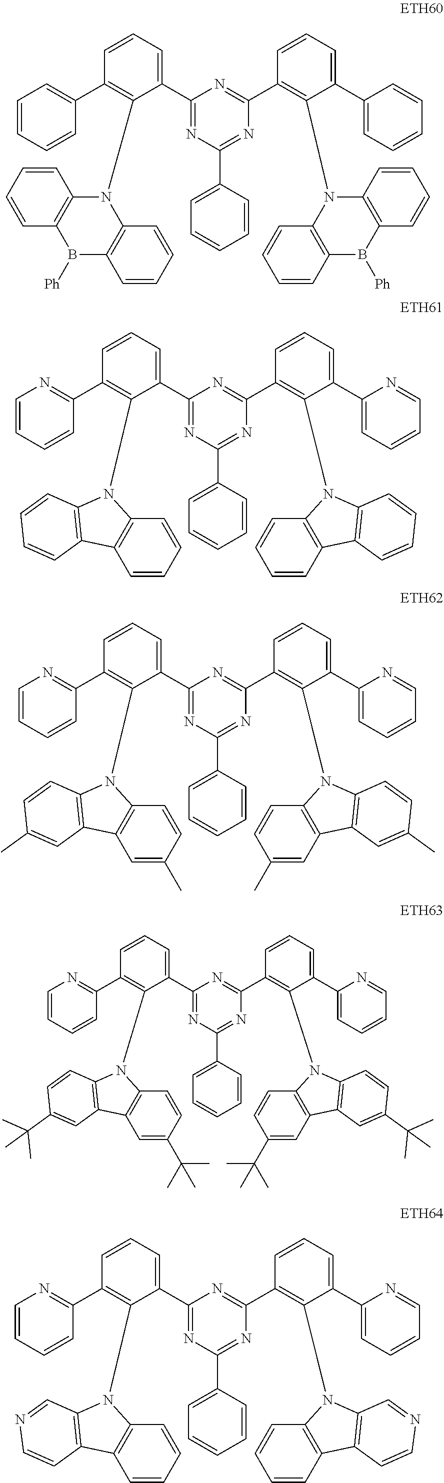

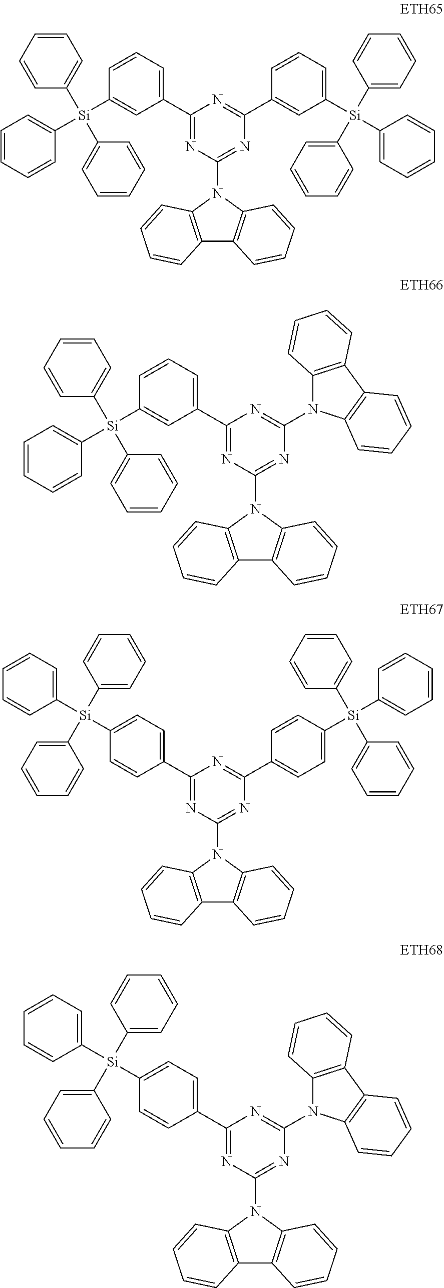 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

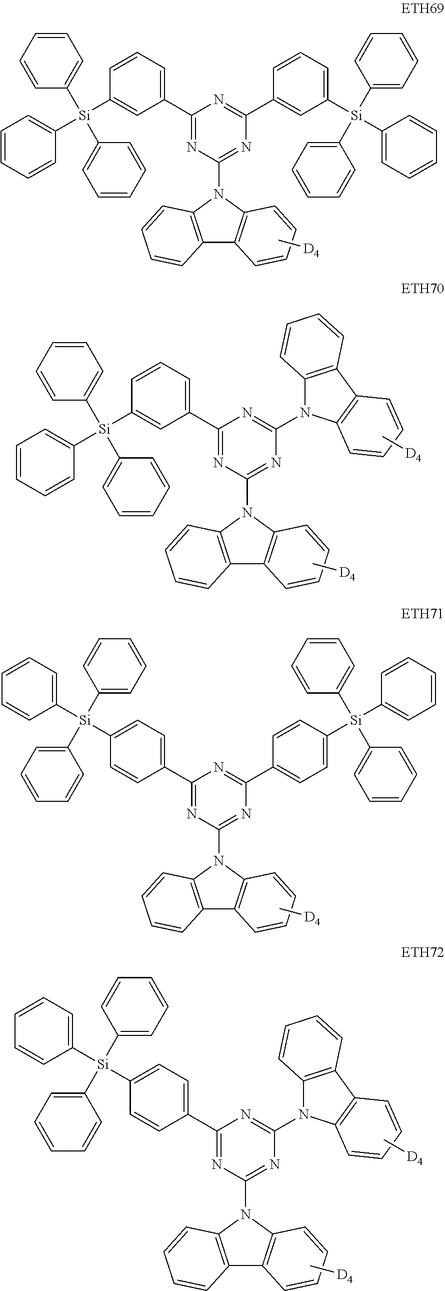

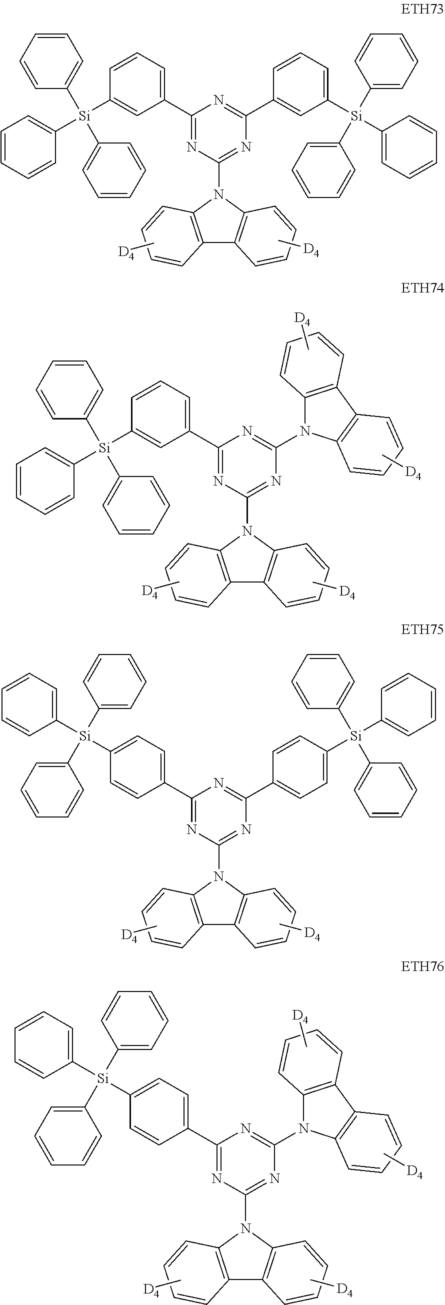

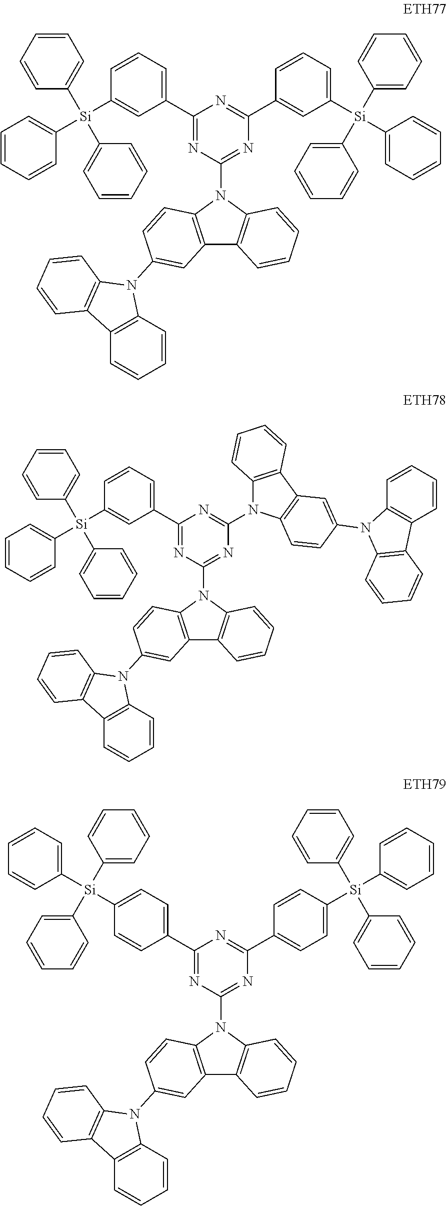 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

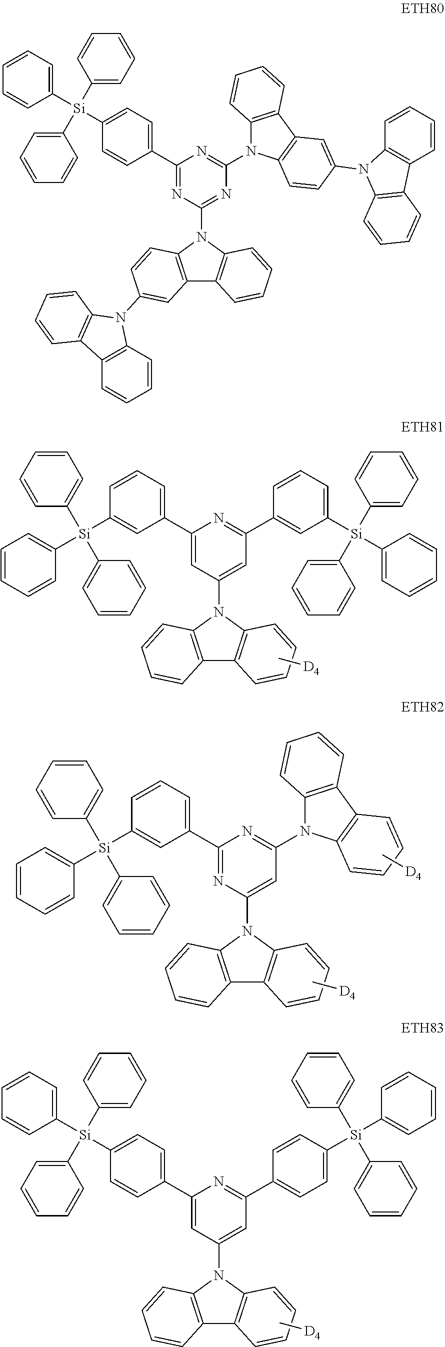

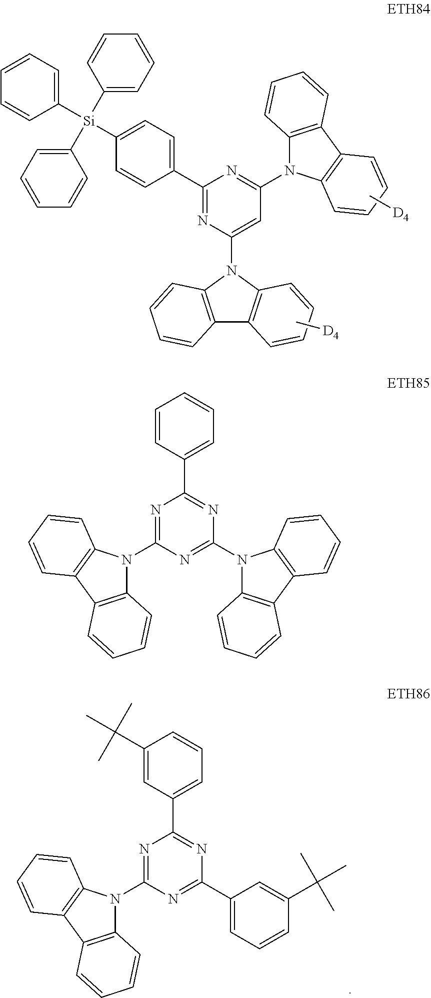

In one or more embodiments, the third compound may be represented by Formula 401 A:

M₄₀₁(L₄₀₁)ₙ₄₀₁(L₄₀₂)ₙ₄₀₂ Formula 401A

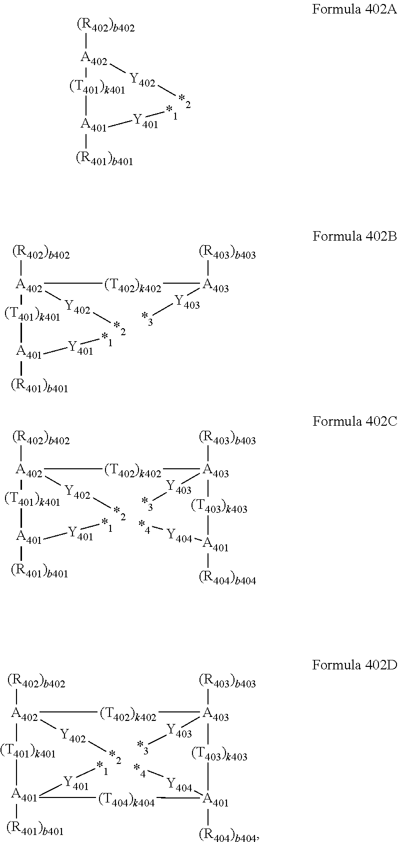

wherein, in Formulae 401A and 402A to 402D,

M₄₀₁ may be a first-row transition metal of the Periodic Table of Elements, a second-row transition metal of the Periodic Table of Elements, or a third-row transition metal of the Periodic Table of Elements,

L₄₀₁ may be a ligand represented by one selected from among Formulae 402A to 402D,

L₄₀₂ may be a monodentate ligand, a bidentate ligand, or a tridentate ligand,

n401 may be 1 or 2,

n402 may be an integer from 0 to 4,

A₄₀₁ to A₄₀₄ may each independently be a C₅-C₃₀ carbocyclic group or a C₁-C₃₀ heterocyclic group,

T₄₀₁ to T₄₀₄ may each independently be a single bond, a double bond, *—O—*′, *—S—*′, *—C(═O)—*′, *—S(═O)—*′, *—C(R₄₀₅)(R₄₀₆)—*′, *—C(R₄₀₅)═C(R₄₀₆)—*′, *—C(R₄₀₅)=*, Si(R₄₀₅)(R₄₀₆)—*′, *—B(R₄₀₅)—*′, *—N(R₄₀₅)—*′, or *—P(R₄₀₅)—*′,

k401 to k404 may each independently be 1, 2, or 3,

Y₄₀₁ to Y₄₀₄ may each independently be a single bond (e.g., a covalent bond or a coordinate bond), *—O—*′, *—S—*′, *—C(R₄₀₇)(R₄₀₈)—*′, *—Si(R₄₀₇)(R₄₀₈)—*′, *—B(R₄₀₇)—*′, *—N(R₄₀₇)—*′, or *—P(R₄₀₇)—*′,

*₁, *₂, *₃, and *₄ each indicate a binding site to M₄₀₁,

R₄₀₁ to R₄₀₈ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

R₄₀₁ to R₄₀₈ may optionally be bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,

b401 to b404 may each independently be an integer from 0 to 10,

* and *′ each indicate a binding site to a neighboring atom, and

Q₁ to Q₃ and R₁₀ₐ are each the same as described herein.

In one or more embodiments, the compound represented by Formula 401A may be a carbene complex.

The term “carbene complex” as utilized herein refers to a complex which includes a metal and a ligand bonded to the metal, wherein at least one bond between the metal and the ligand is a bond between the metal and carbon of carbene.

In one or more embodiments, the sensitizer may include the compound represented by Formula 401A.

In one or more embodiments, the third compound may include at least one selected from among Compounds PD1 to PD41, but embodiments of the present disclosure are not limited thereto:

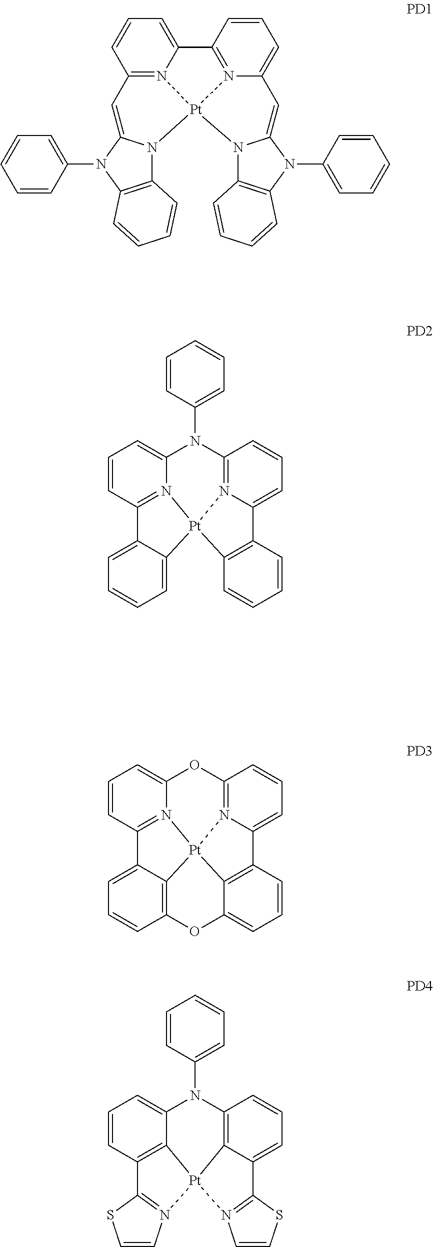

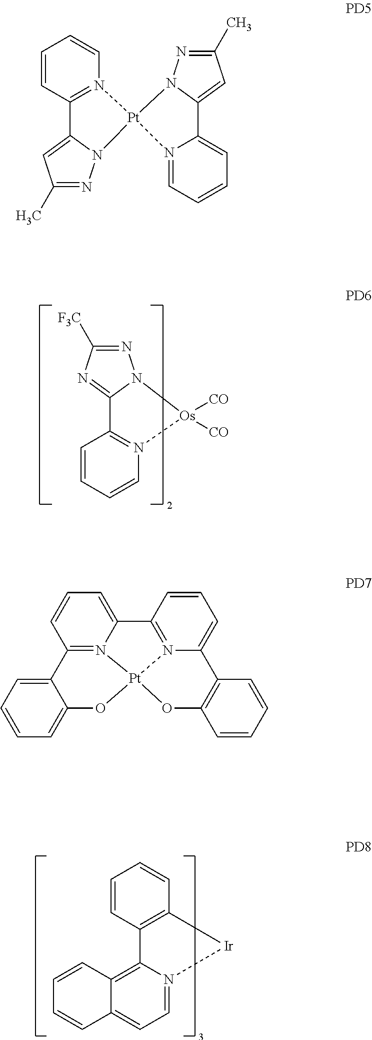

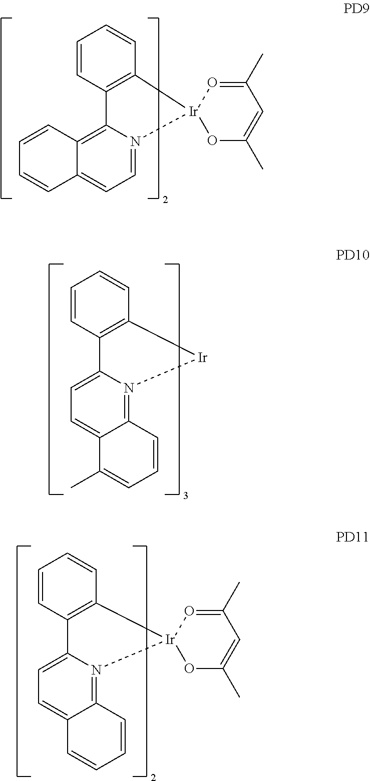

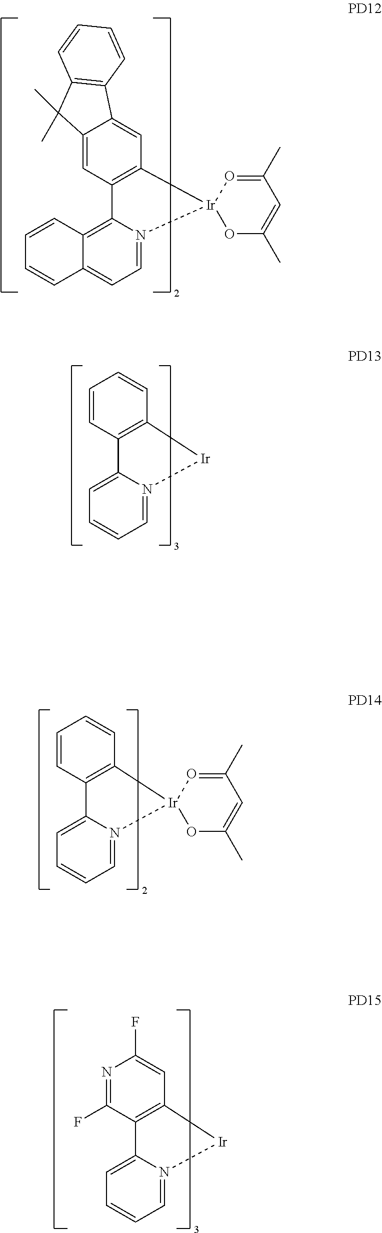

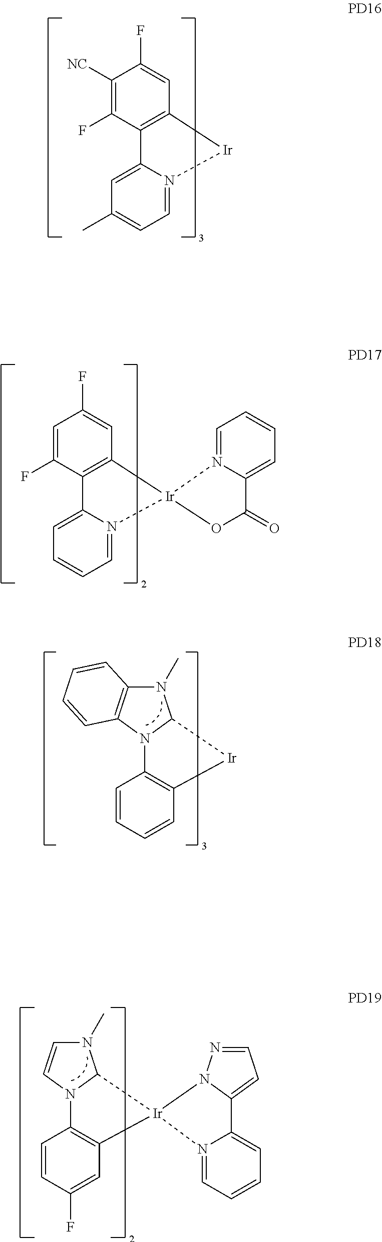

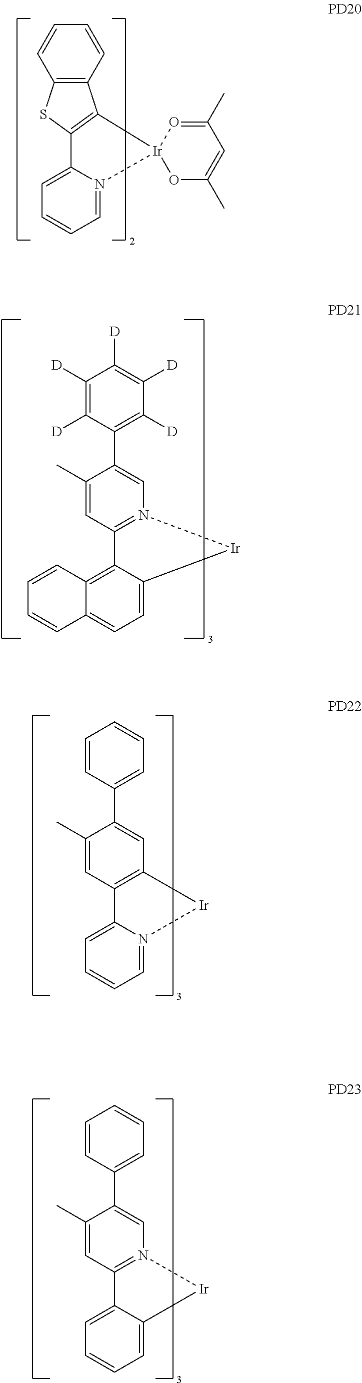

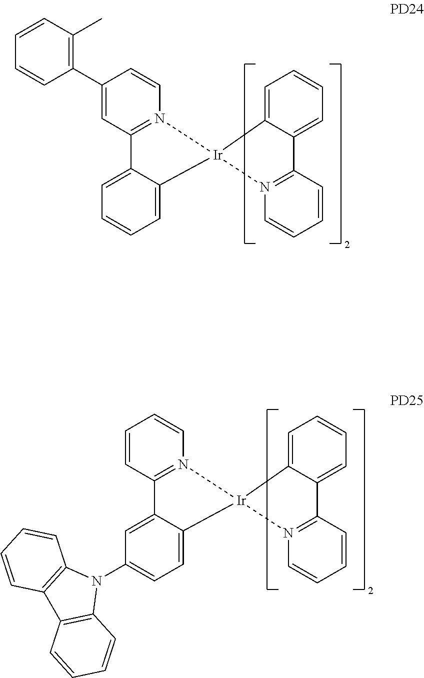

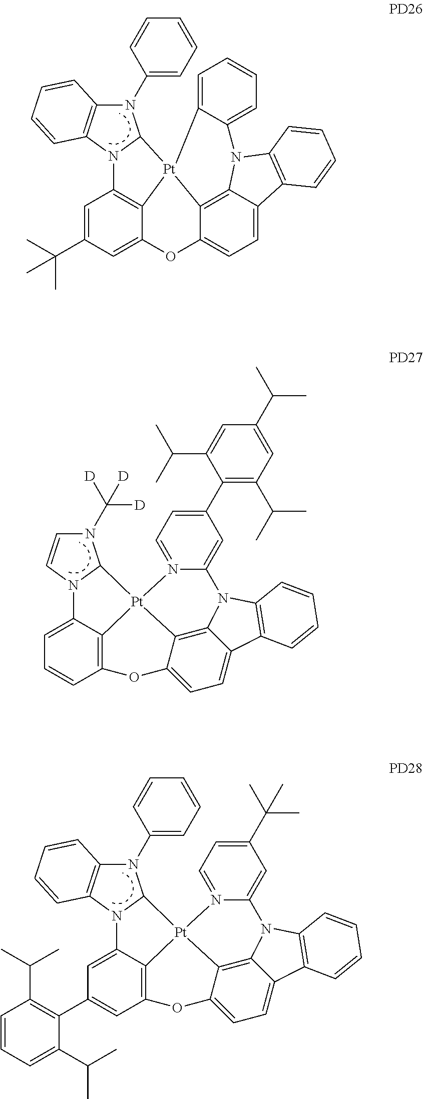

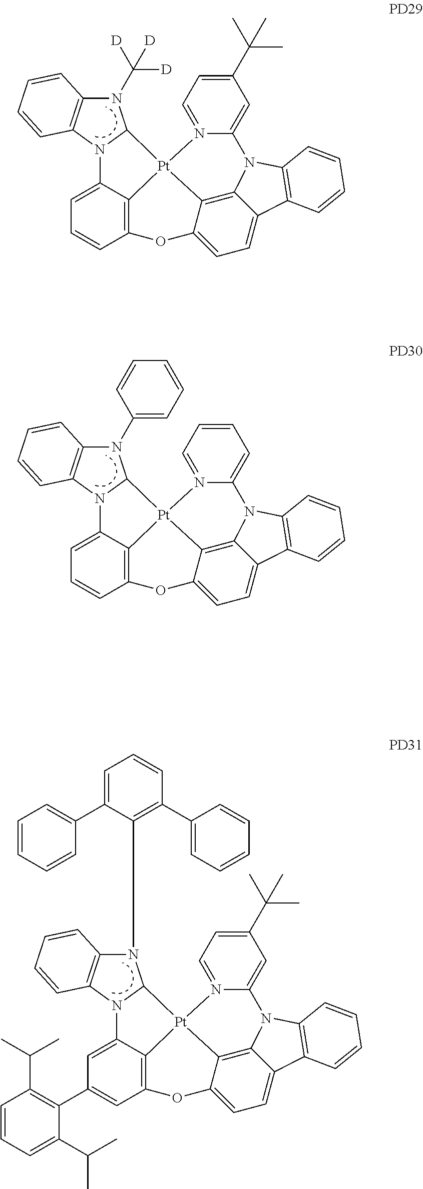

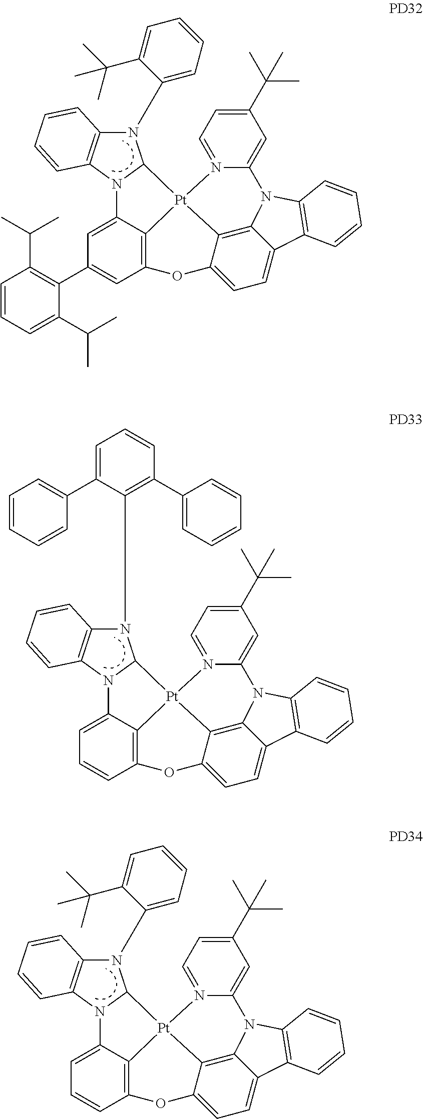

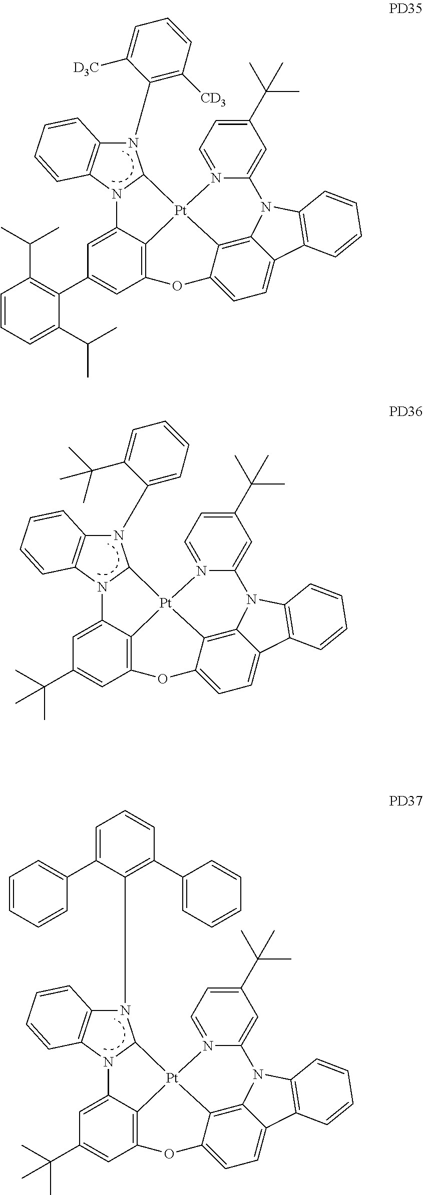

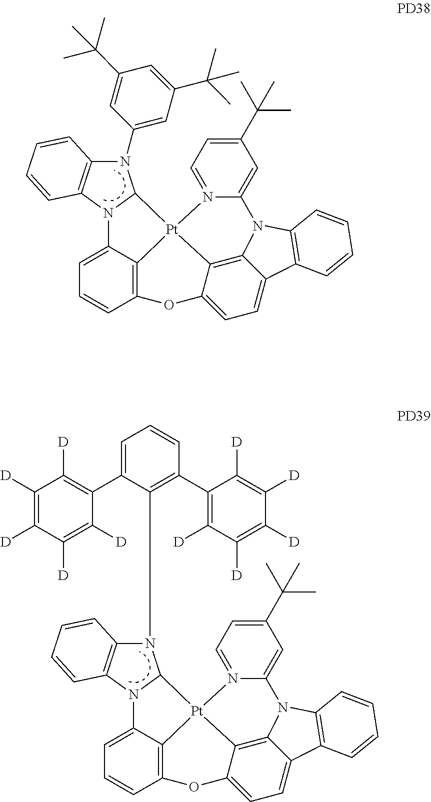

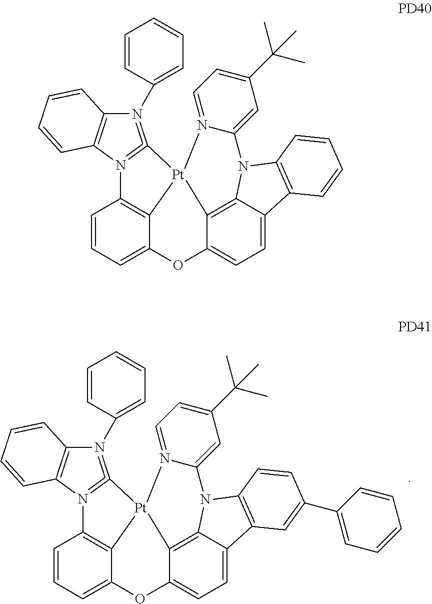

In one or more embodiments, R₃₀₁ to R₃₀₃, R₃₀₄ₐ to R₃₀₆ₐ, R_{304b} to R_{306b}, and R₃₁₁ to R₃₁₄ in Formulae 301-1A and 301-2A, R₃₂₁ to R₃₂₆ in Formula 302, and R₄₀₁ to R₄₀₈ in Formulae 401A and 402A to 402D may each independently be: hydrogen, deuterium, —F, —Cl, —Br, —I, a hydroxyl group, a cyano group, a nitro group, an amidino group, a hydrazine group, a hydrazone group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a benzoisoquinolinyl group, a phthalazinyl group, a naphthylidinyl group, a quinoxalinyl group, a benzoquinoxalinyl group, a quinazolinyl group, a cinnolinyl group, a carbazolyl group, a phenanthridinyl group, an acridinyl group, a phenanthrolinyl group, a phenazinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a thiadiazolyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a benzoisoquinolinyl group, a phthalazinyl group, a naphthylidinyl group, a quinoxalinyl group, a benzoquinoxalinyl group, a quinazolinyl group, a cinnolinyl group, a carbazolyl group, a phenanthridinyl group, an acridinyl group, a phenanthrolinyl group, a phenazinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a thiadiazolyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, —Si(Q₃₁)(Q₃₂)(Q₃₃), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and

Q₁ to Q₃ and Q₃₁ to Q₃₃ are each the same as described herein.

In one or more embodiments, R₃₀₁ to R₃₀₃, R₃₀₄ₐ to R₃₀₆ₐ, R_{304b} to R_{306b}, and R₃₁₁ to R₃₁₄ in Formulae 301-1A to 301-2A, R₃₂₁ to R₃₂₆ in Formula 302, and R₄₀₁ to R₄₀₈ in Formulae 401A and 402A to 402D may each independently be:

hydrogen, deuterium, —F, —Cl, —Br, —I, —CD₃, —CD₂H, —CDH₂, —CF₃, —CF₂H, —CFH₂, a cyano group, a nitro group, a C₁-C₆₀ alkyl group, a C₂-C₆₀ alkenyl group, a C₂—Co alkynyl group, or a C₁-C₆₀ alkoxy group;

a group represented by one of (e.g., one selected from among) Formulae 9-1 to 9-61, or a group represented by one of (e.g., one selected from among) Formulae 10-1 to 10-348; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)Q₁)(Q₂):

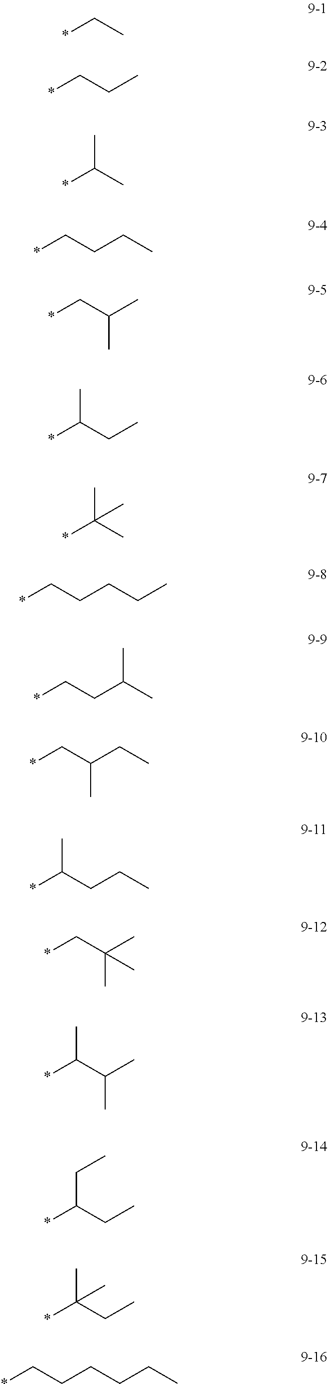

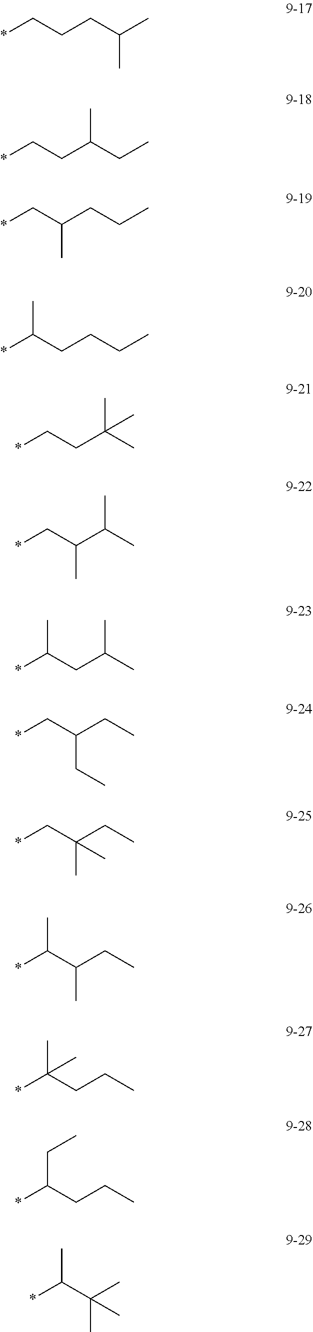

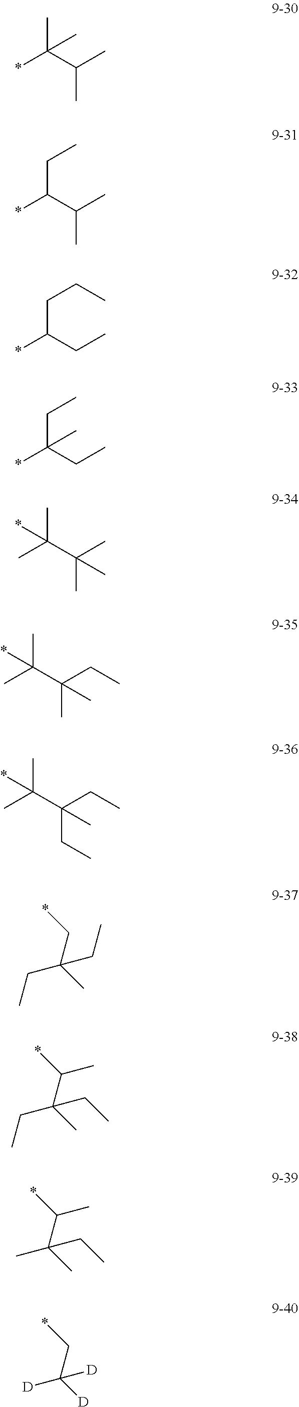

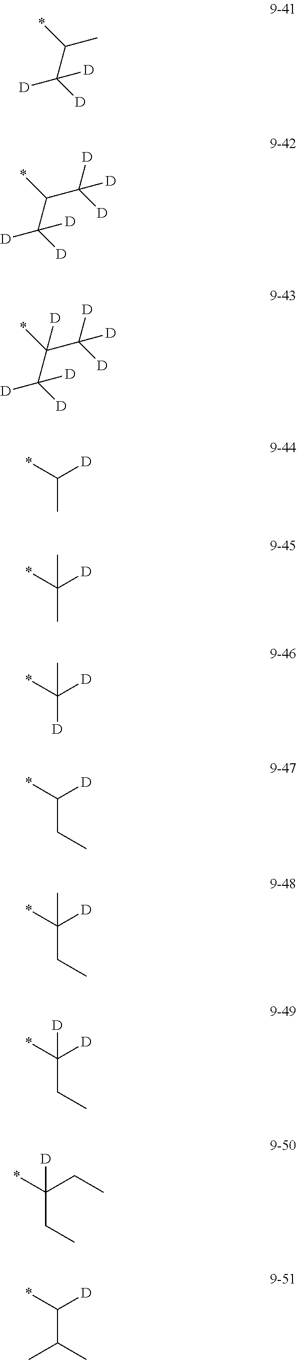

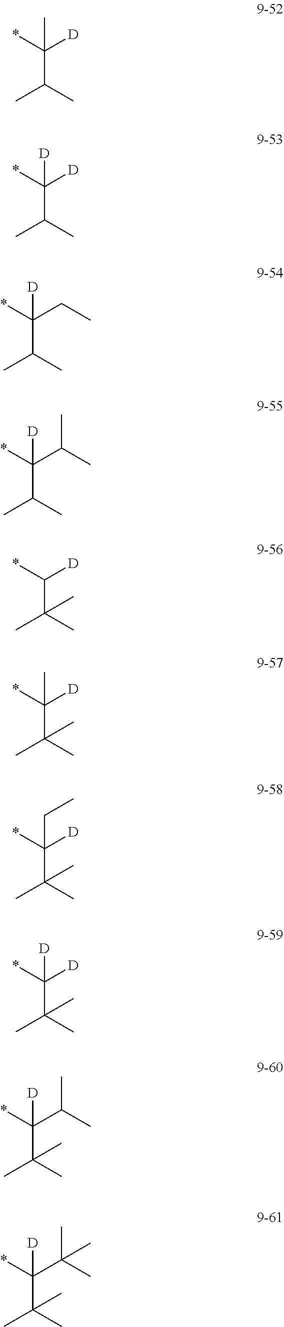

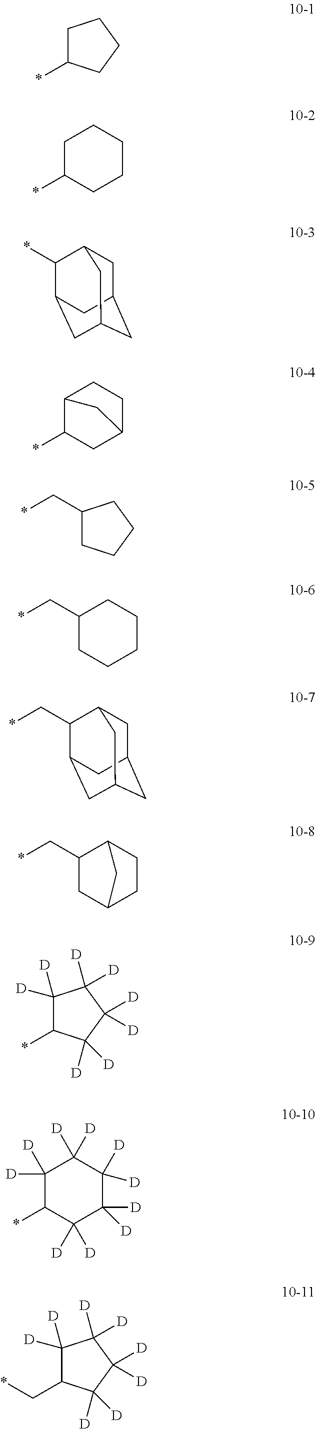

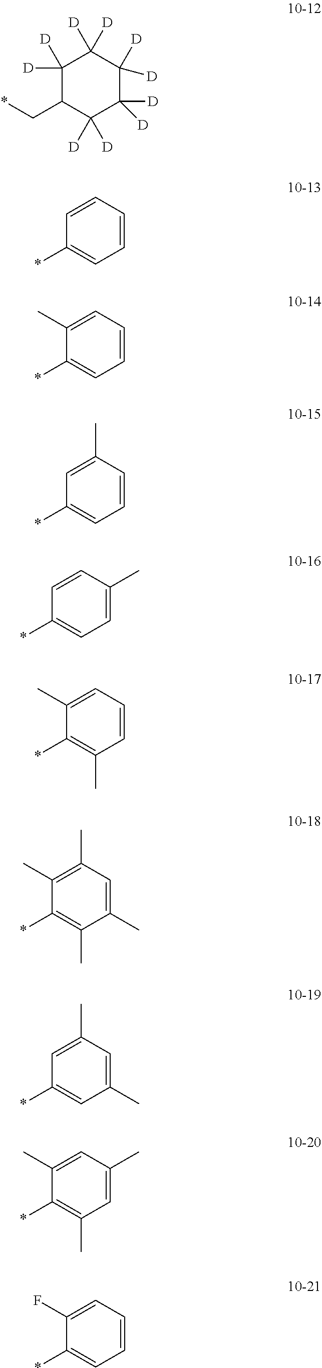

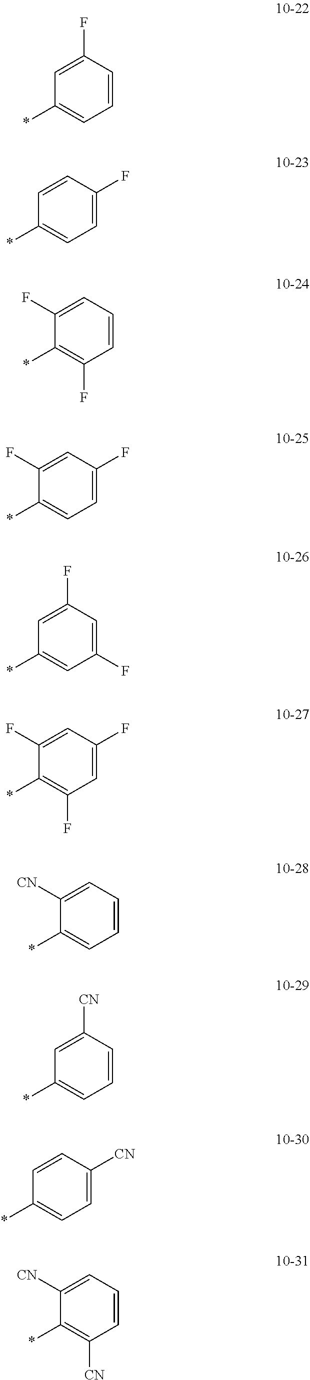

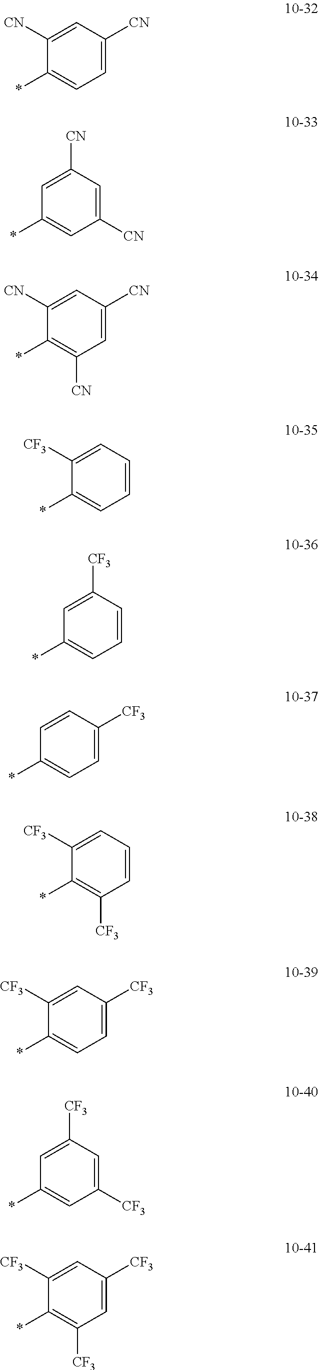

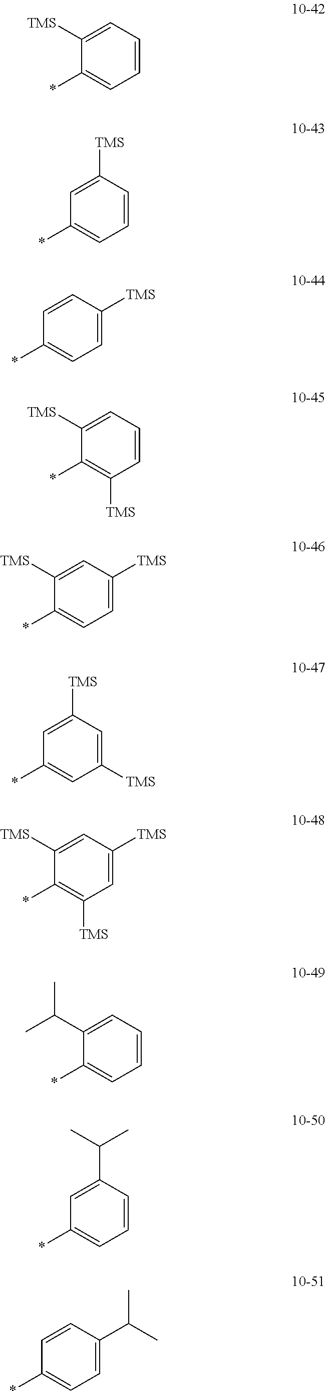

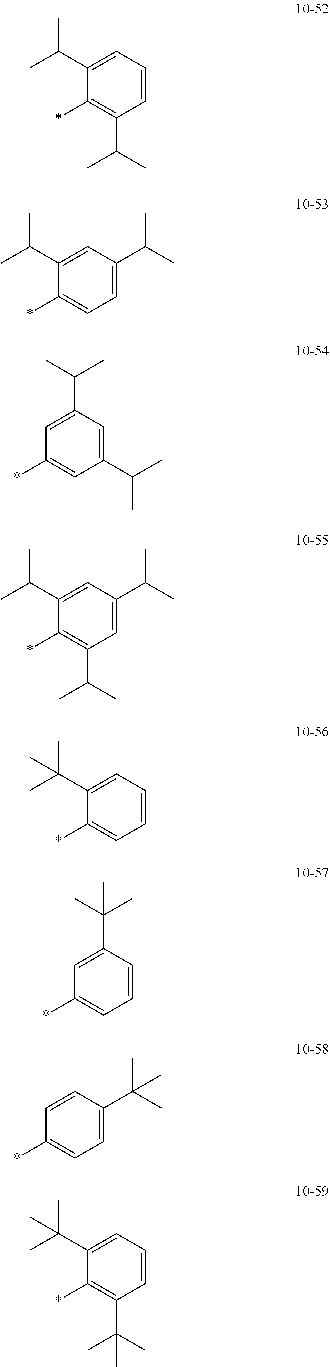

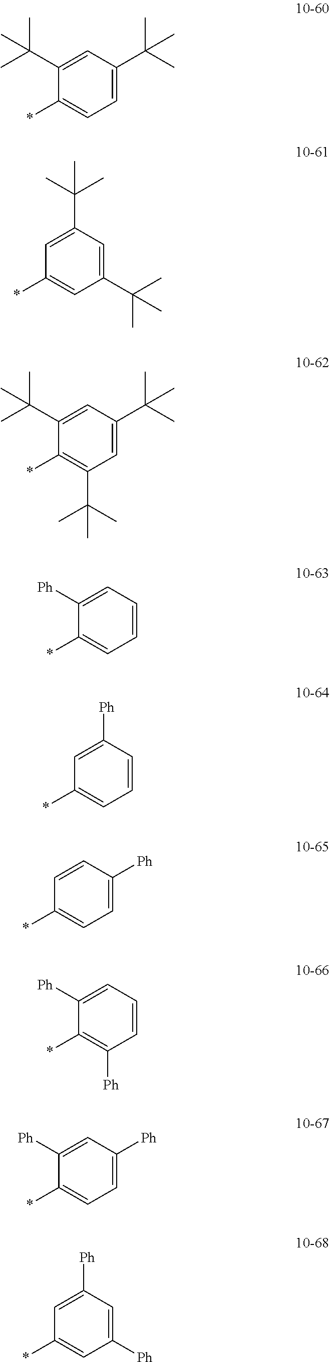

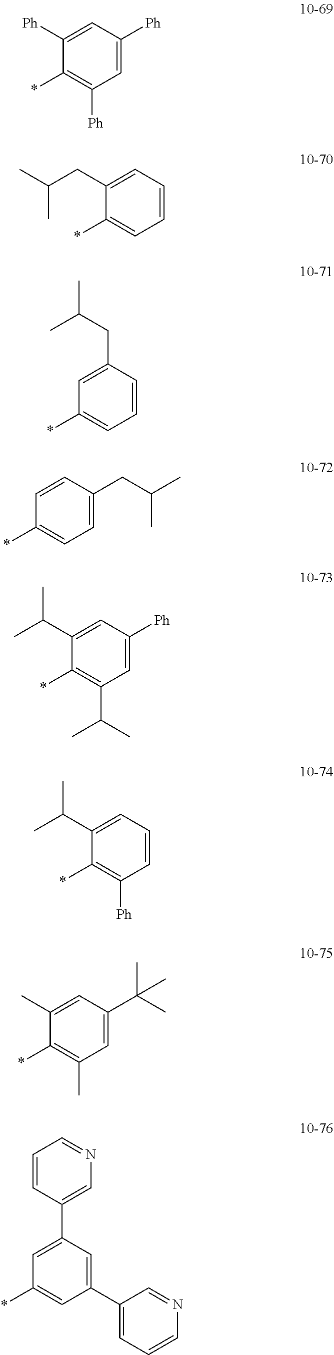

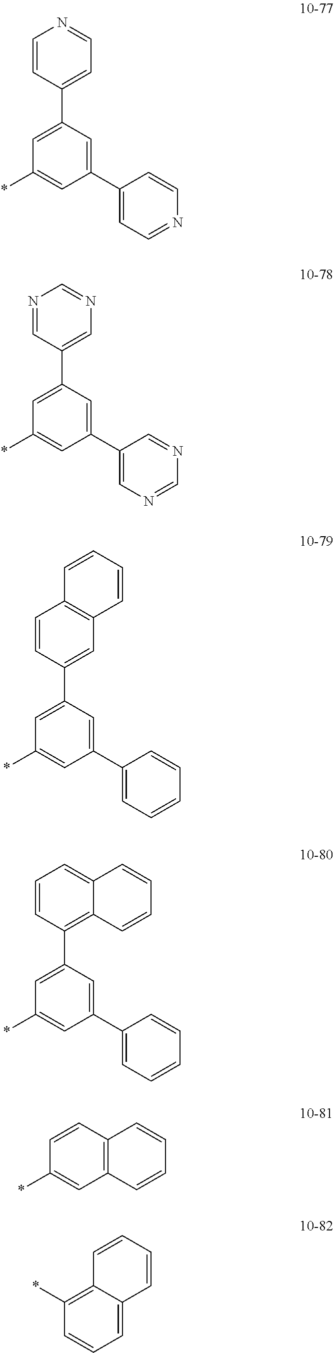

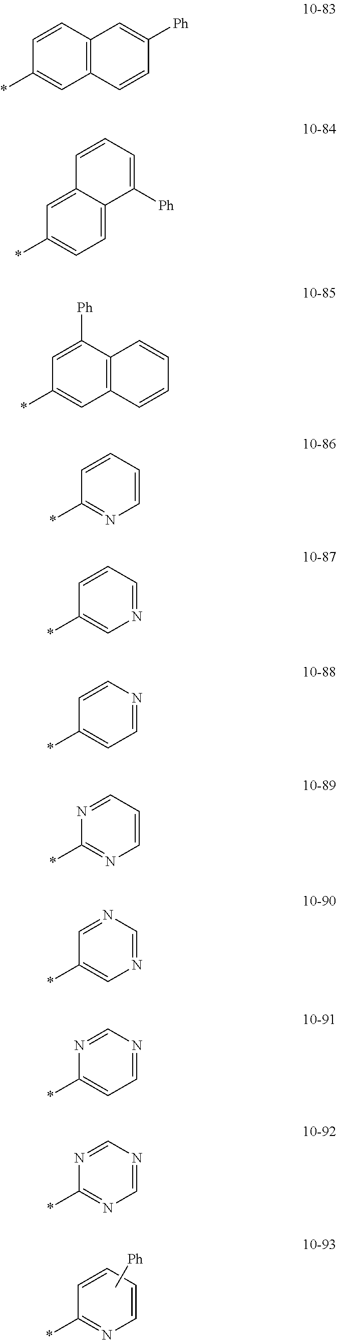

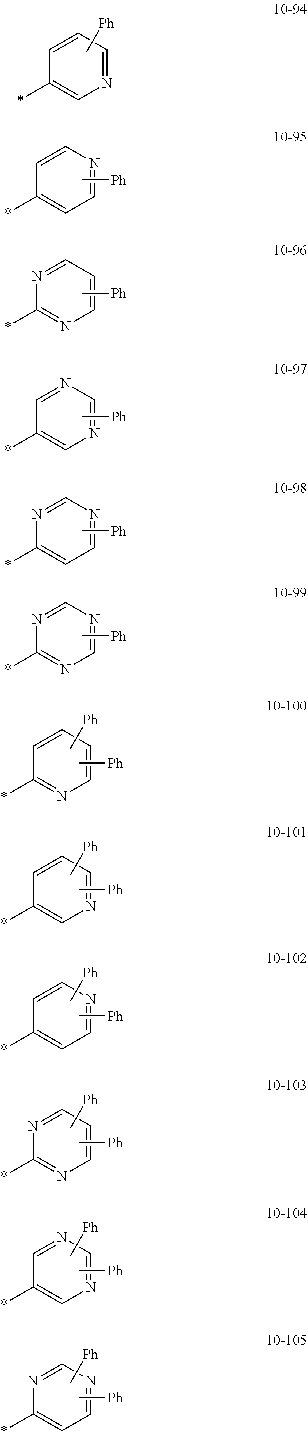

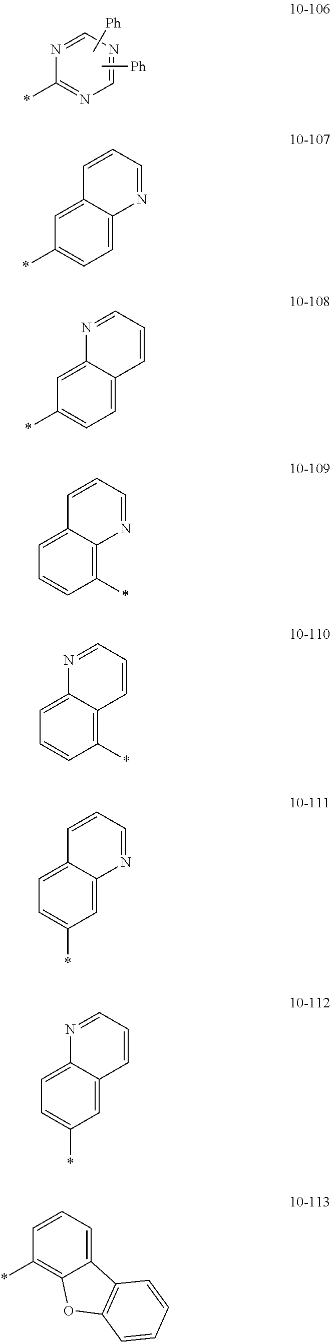

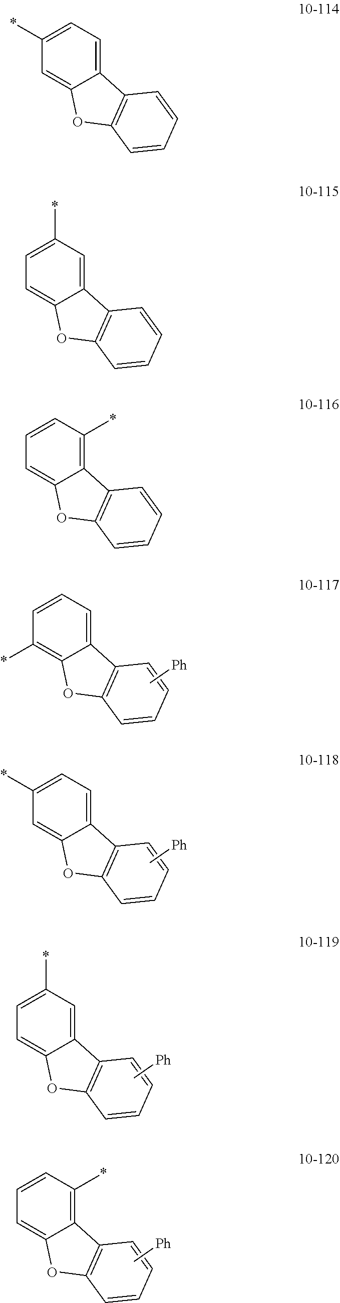

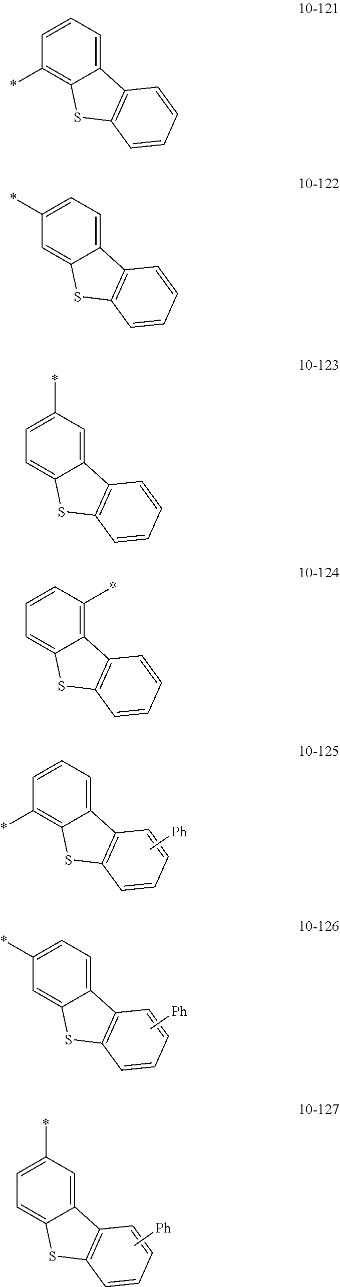

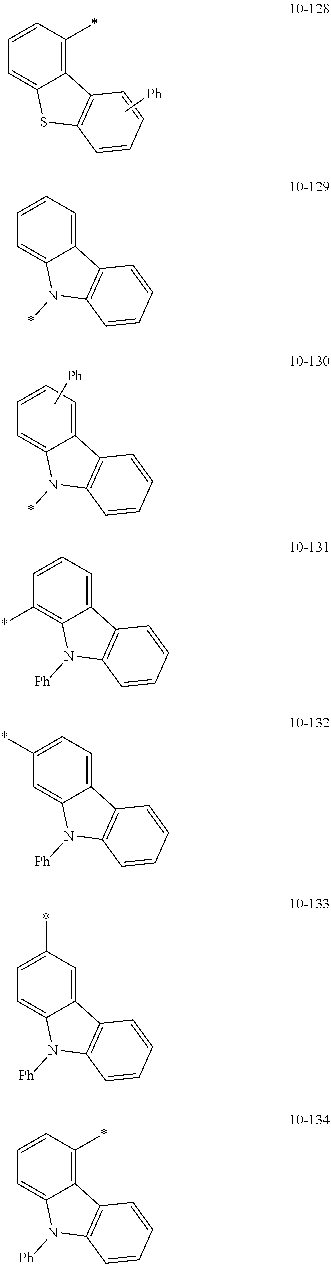

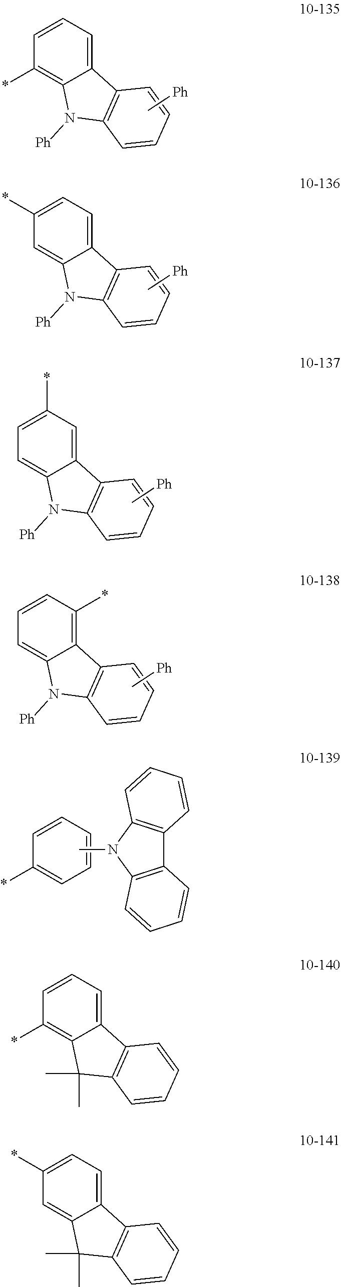

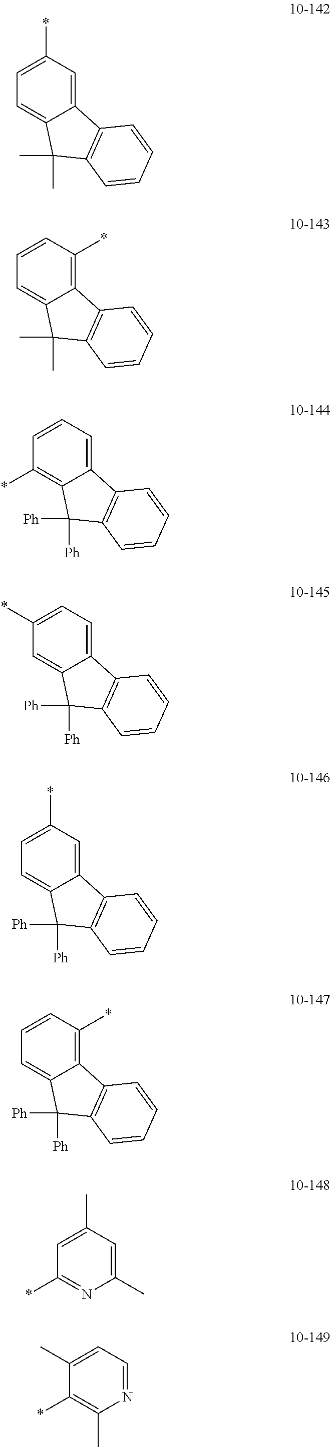

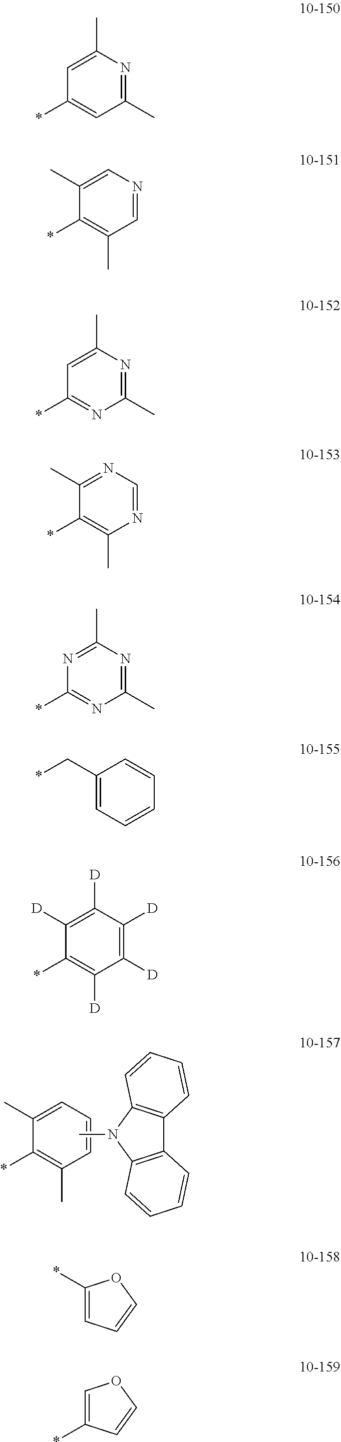

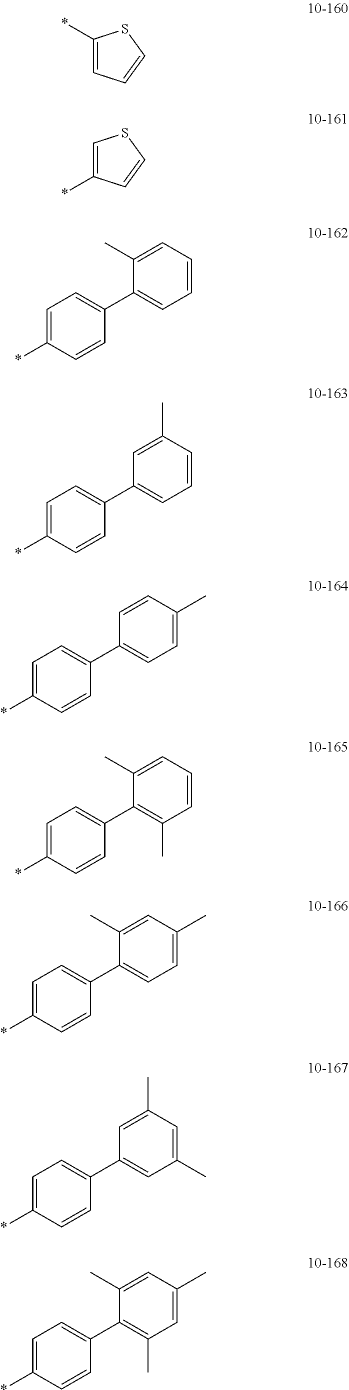

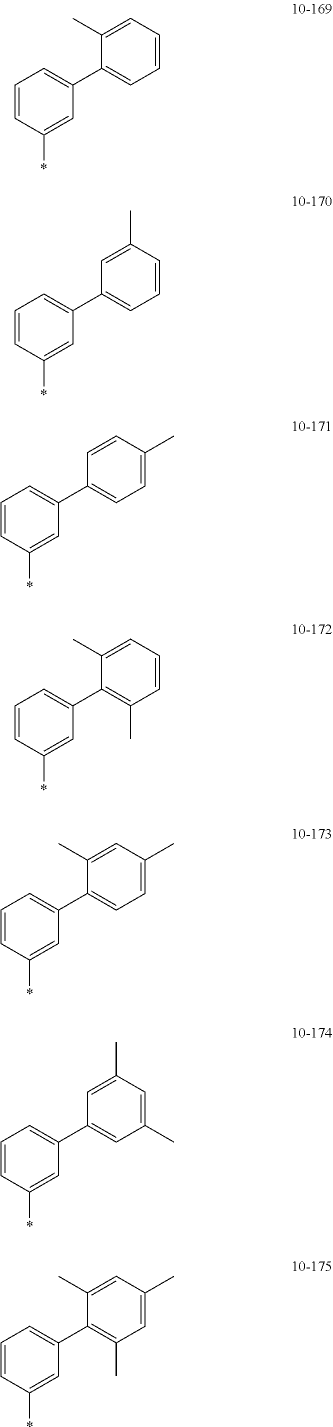

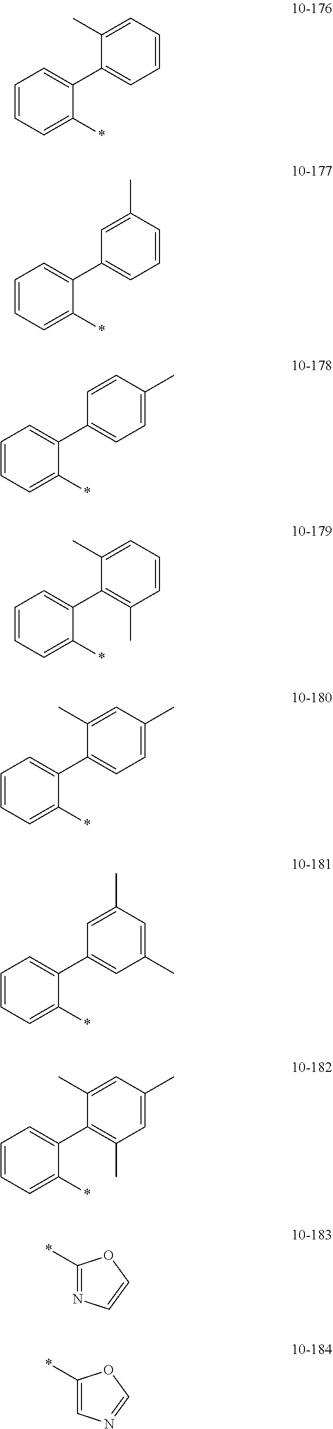

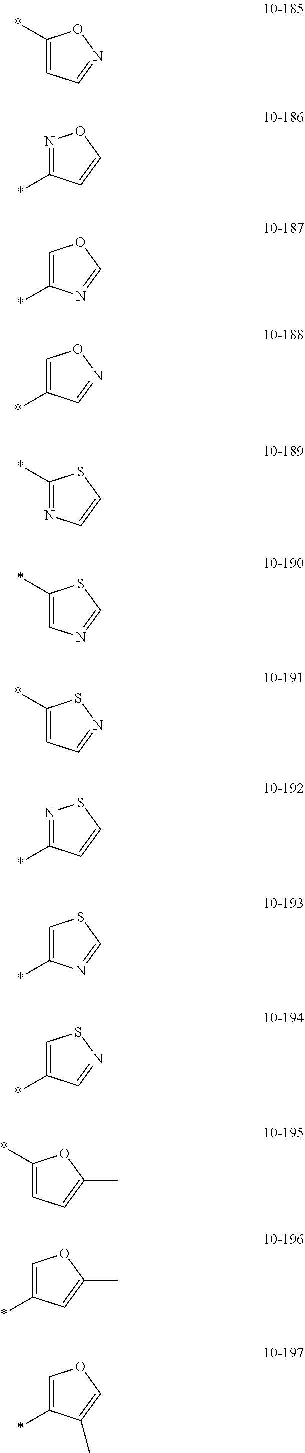

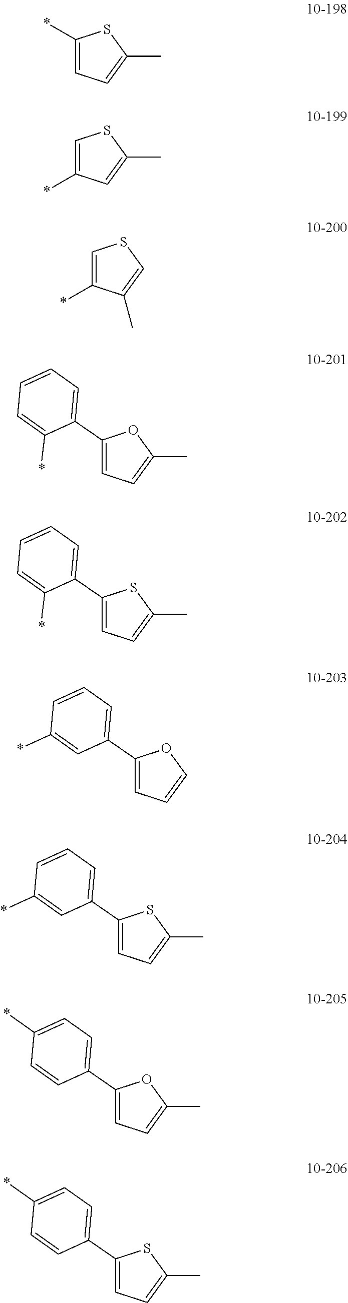

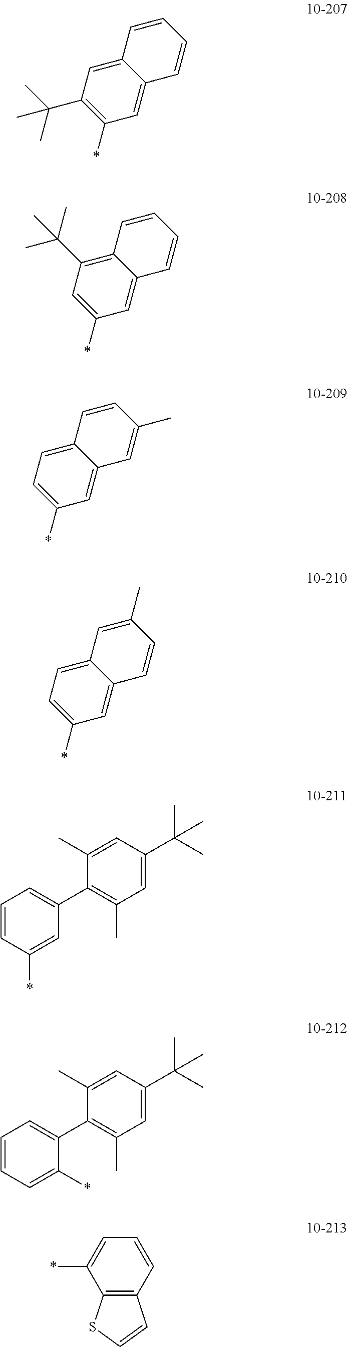

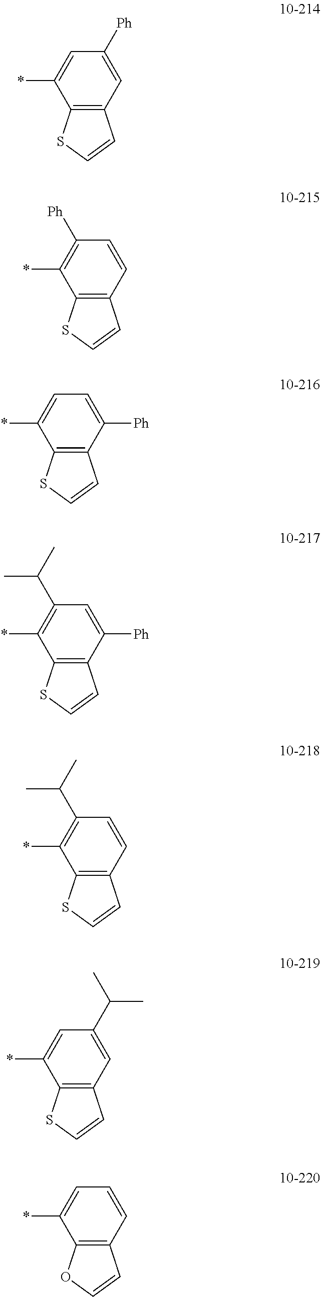

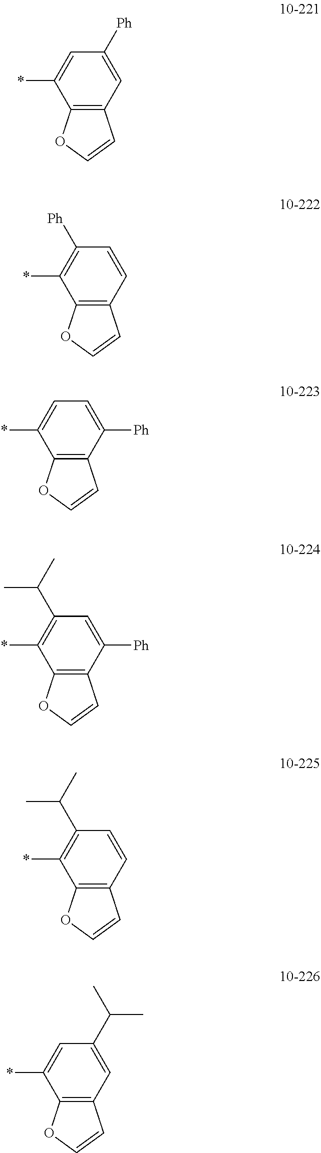

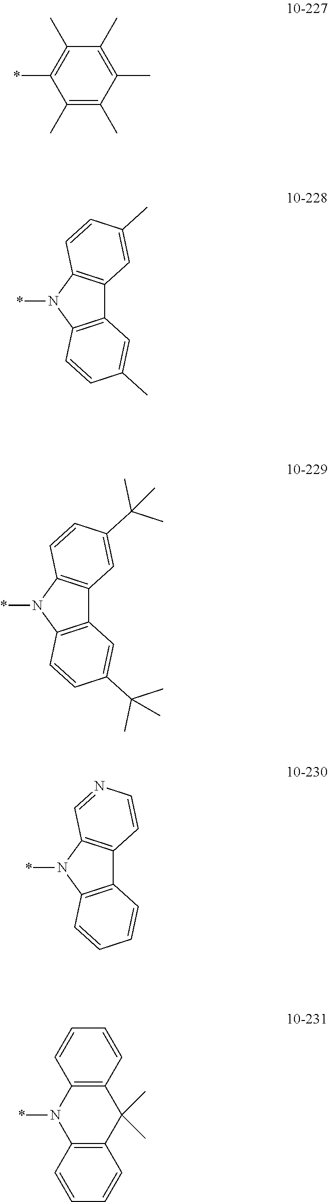

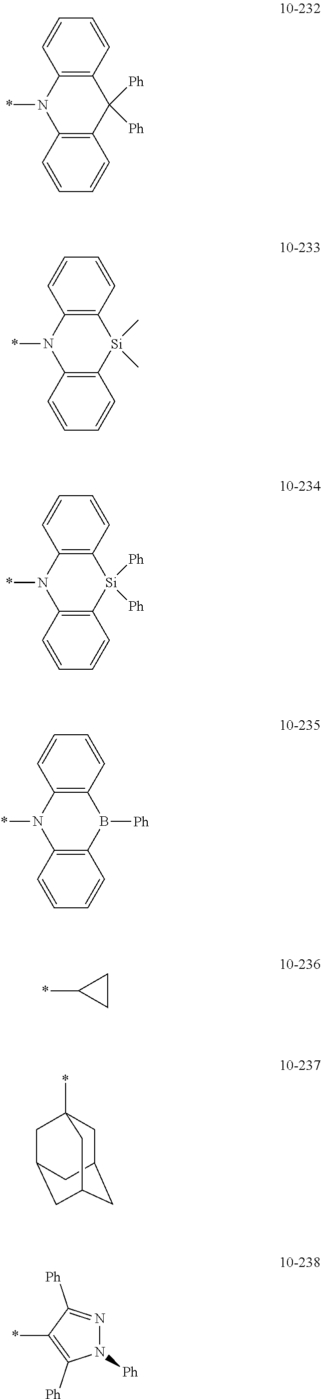

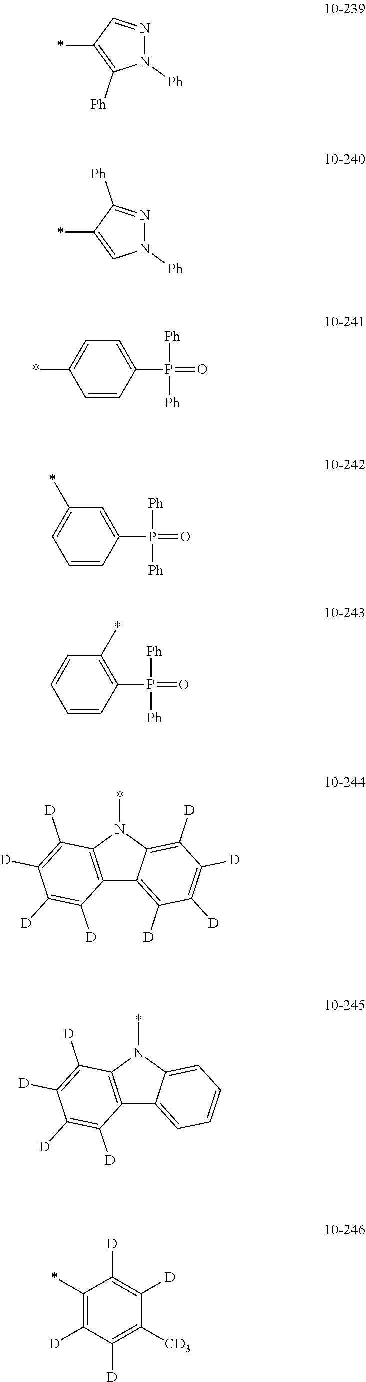

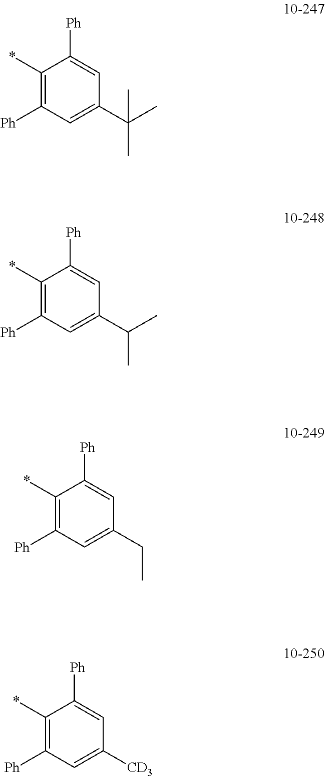

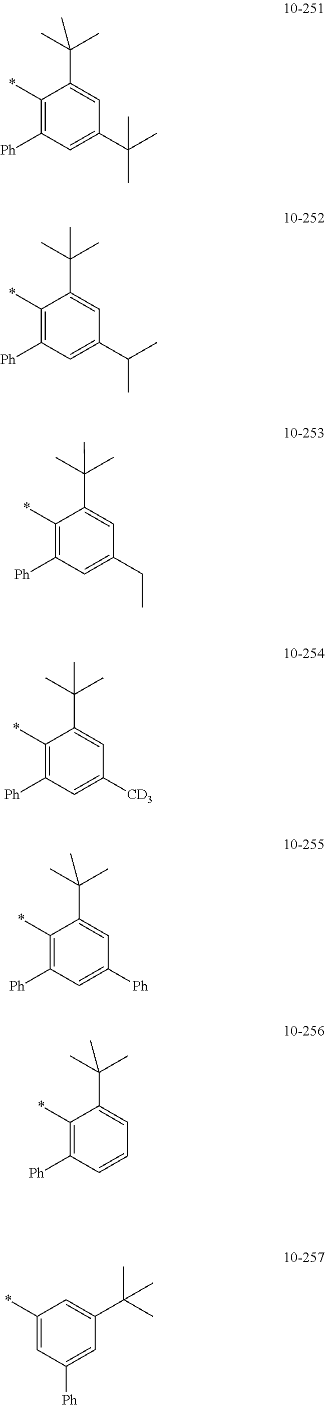

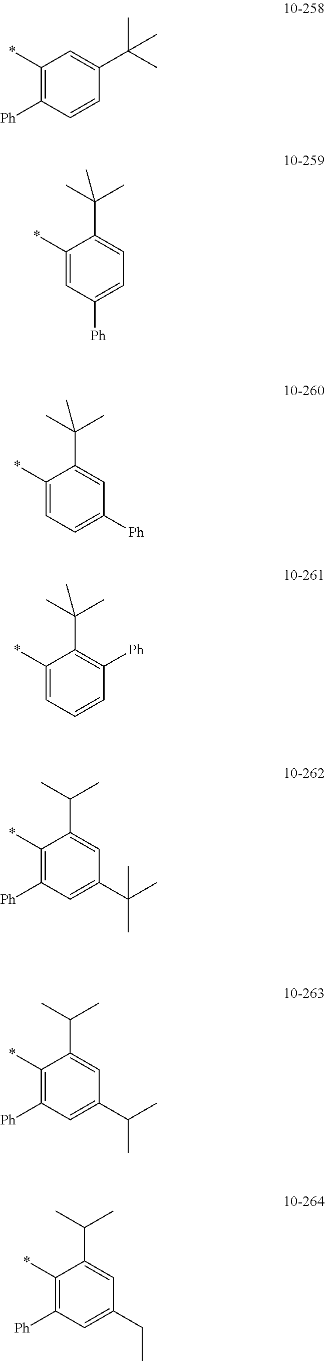

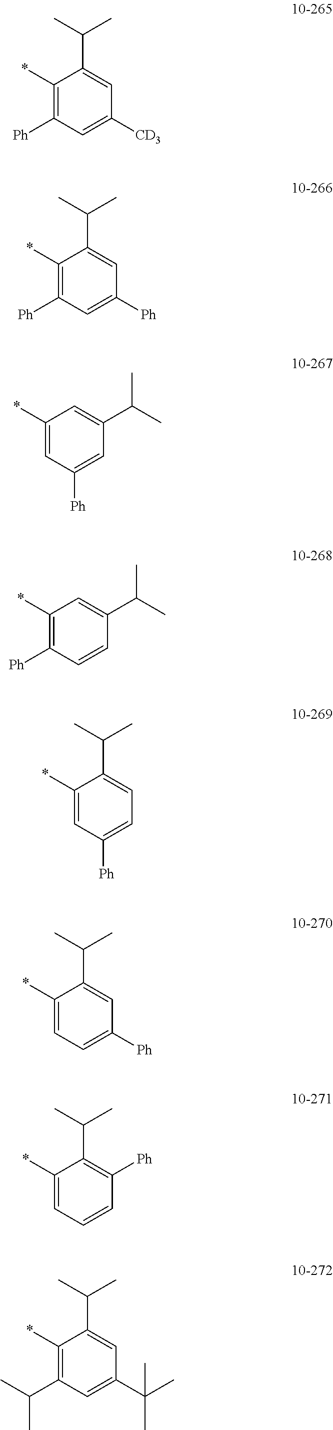

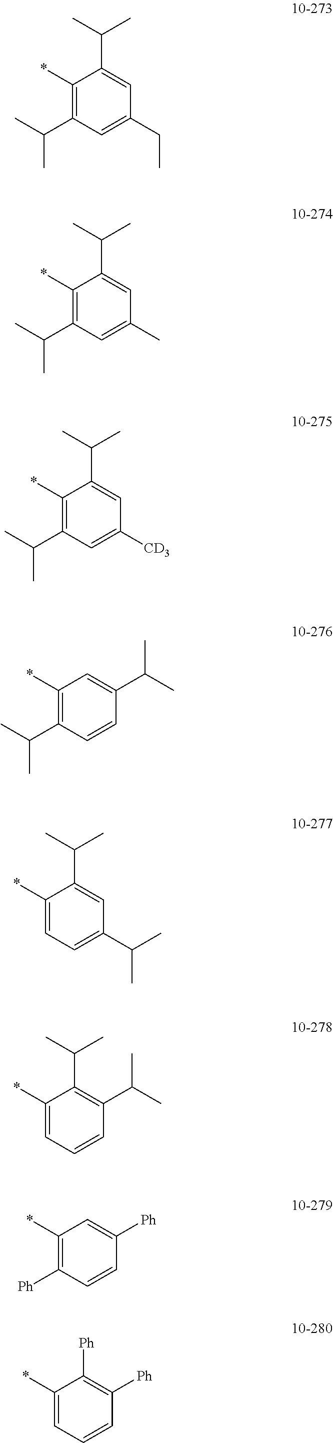

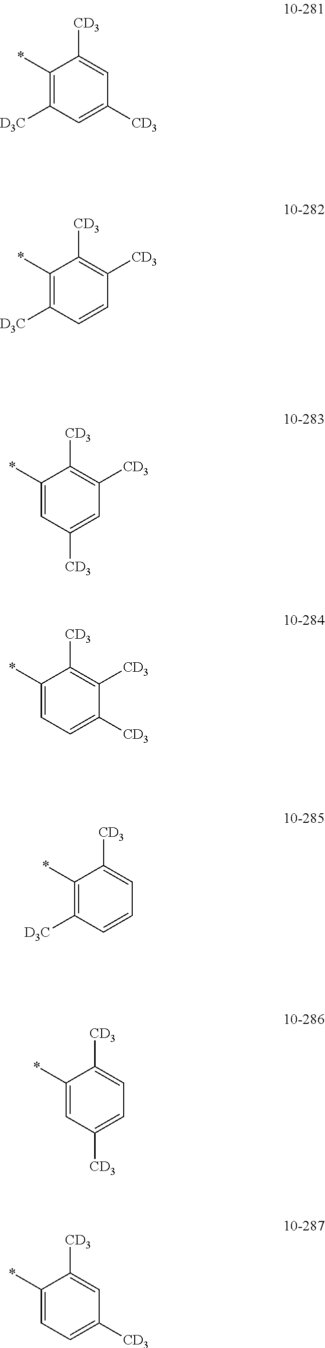

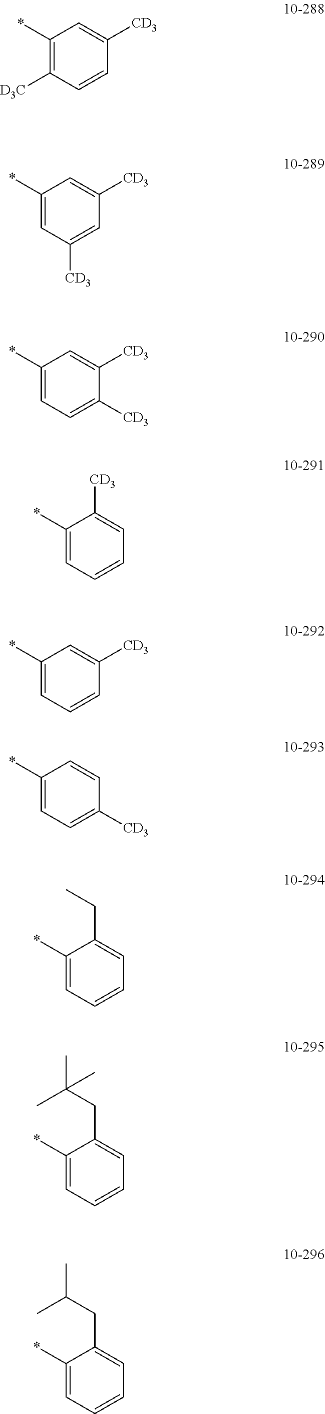

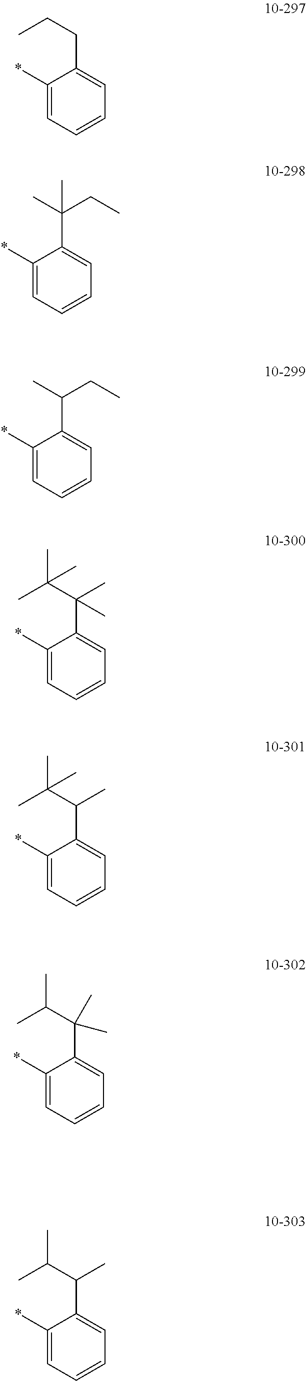

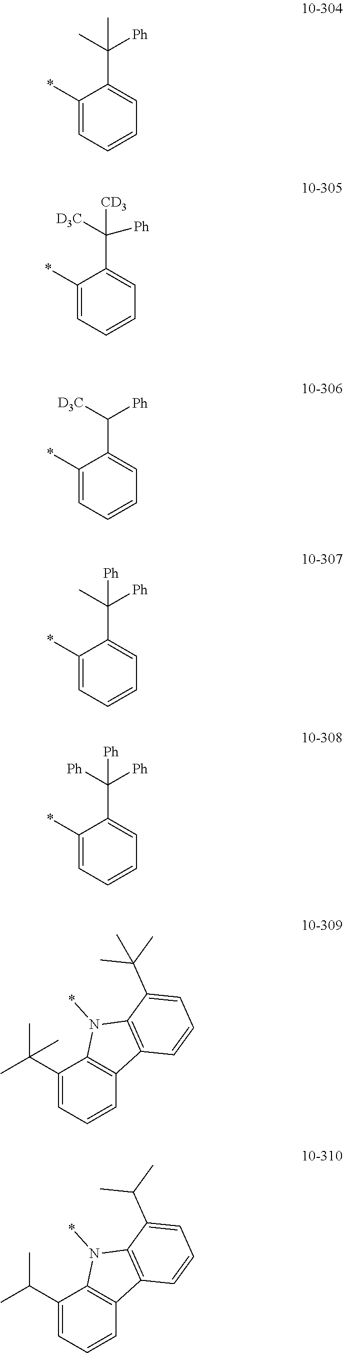

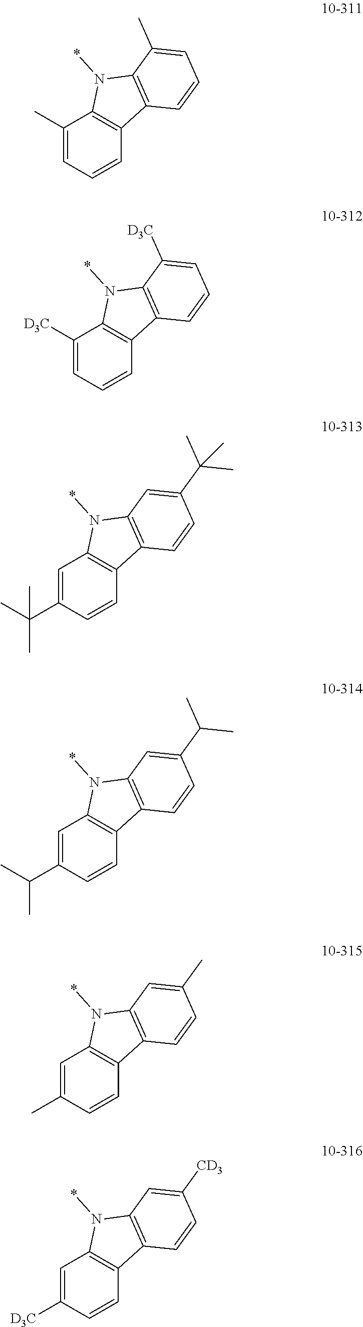

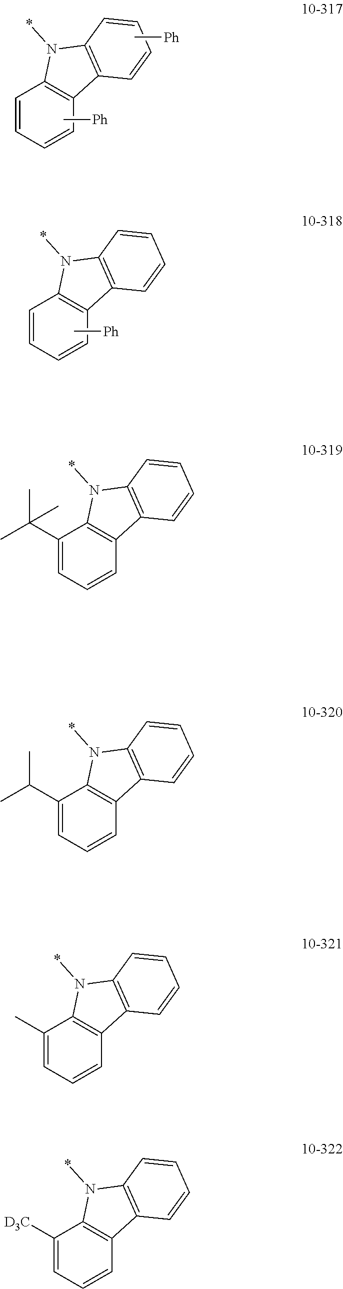

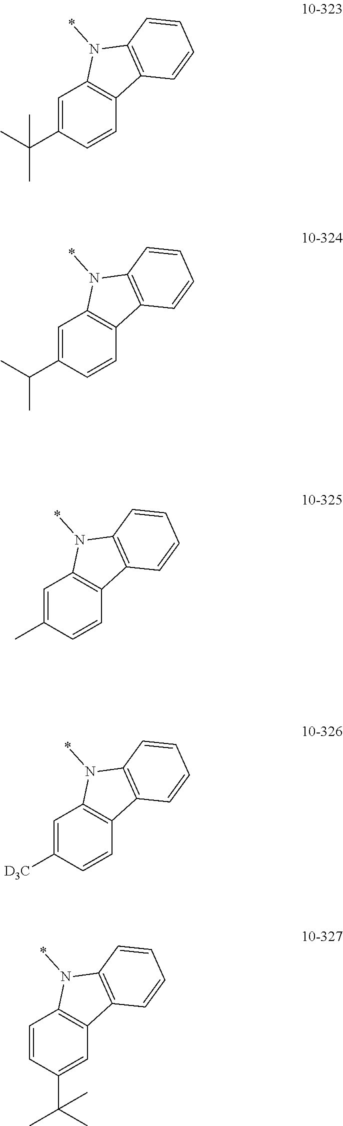

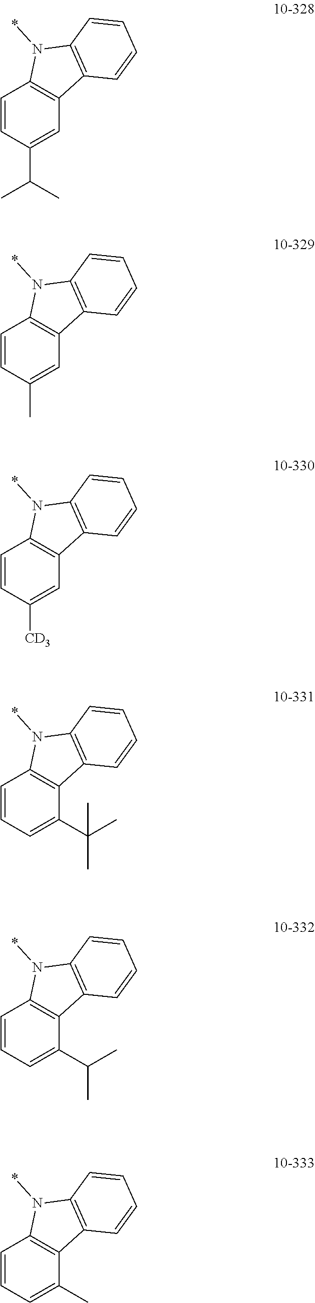

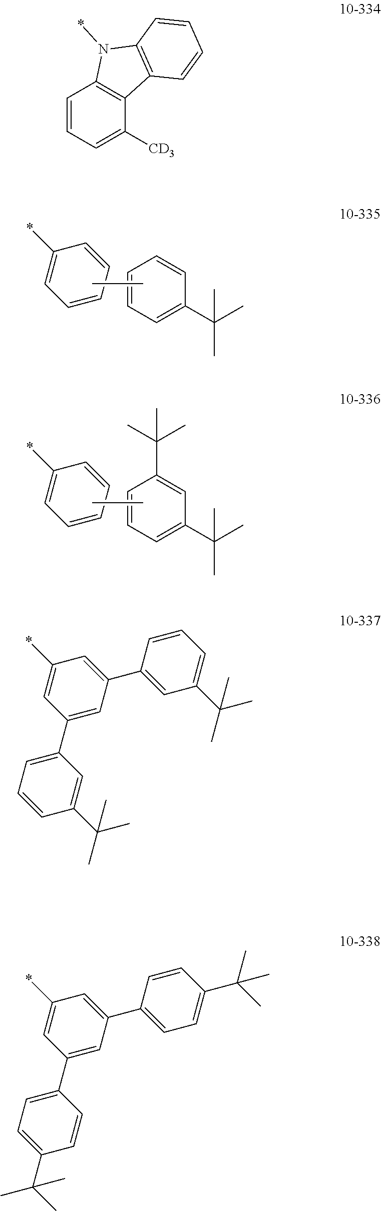

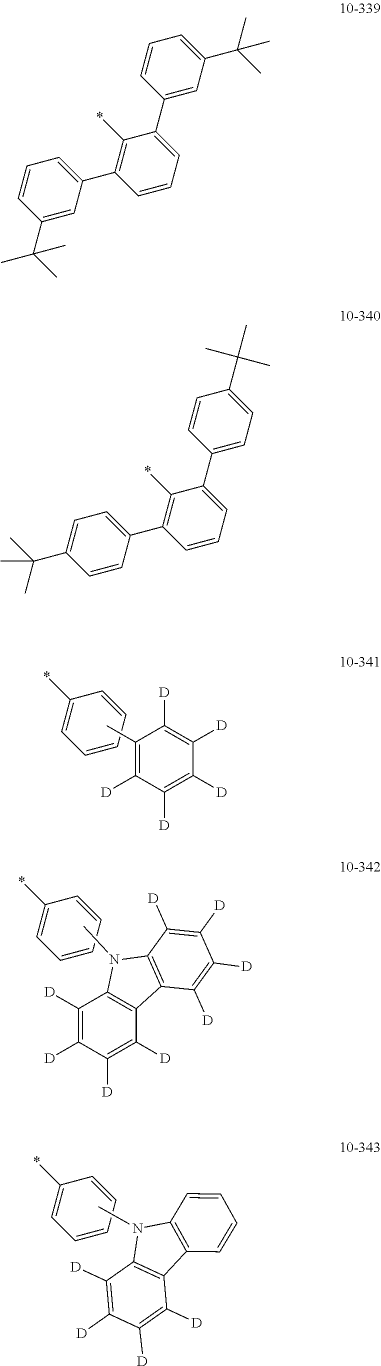

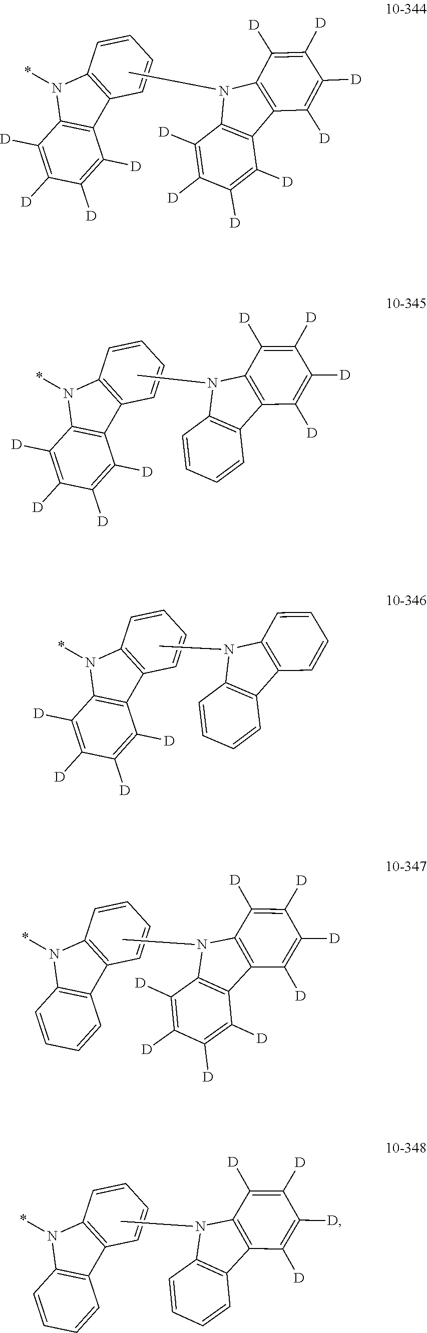

wherein, in Formulae 9-1 to 9-61 and 10-1 to 10-348, * indicates a binding site to a neighboring atom, “Ph” represents a phenyl group, and “TMS” represents a trimethylsilyl group.

Q₁ to Q₃ are each the same as described herein.

In one or more embodiments, the electron transport region of the light-emitting device may include a hole-blocking layer, and the hole-blocking layer may include a phosphine oxide-containing compound, a silicon-containing compound, or any combination thereof. In one or more embodiments, the hole blocking layer may directly contact the emission layer.

In one or more embodiments, the light-emitting device may include a capping layer located outside (e.g., on) the first electrode or outside (e.g., on) the second electrode.

In one or more embodiments, the light-emitting device may further include at least one of a first capping layer located outside (e.g., on) the first electrode or a second capping layer located outside (e.g., on) the second electrode, and at least one of the first capping layer or the second capping layer may include the amine-containing compound represented by Formula 1. More details for the first capping layer and/or second capping layer may each independently be the same as described elsewhere in the present disclosure.

In one or more embodiments, the light-emitting device may further include:

A first capping layer located outside (e.g., on) the first electrode and including the amine-containing compound represented by Formula 1;

a second capping layer located outside (e.g., on) the second electrode and including the amine-containing compound represented by Formula 1; or

the first capping layer and the second capping layer.

The wording “(interlayer and/or capping layer) includes an amine-containing compound” as utilized herein may be understood as “(interlayer and/or capping layer) may include one kind of amine-containing compound represented by Formula 1 or two or more different kinds of amine-containing compounds, each represented by Formula 1”.

For example, the interlayer and/or the capping layer may include Compound 1 only as the amine-containing compound. In this regard, Compound 1 may be present in the emission layer of the light-emitting device. In some embodiments, the interlayer may include, as the amine-containing compound, Compound 1 and Compound 2. In this regard, Compound 1 and Compound 2 may be present in substantially the same layer (for example, all of Compound 1 and Compound 2 may be present in the emission layer), or may be present in different layers (for example, Compound 1 may be present in the emission layer, and Compound 2 may be present in the electron transport region).

The term “interlayer” as utilized herein refers to a single layer and/or all layers between a first electrode and a second electrode of a light-emitting device.

one or more aspects of embodiments of present disclosure are directed toward an electronic apparatus including the light-emitting device. The electronic apparatus may further include a thin-film transistor. For example, in one or more embodiments, the electronic apparatus may further include a thin-film transistor including a source electrode and a drain electrode, wherein the first electrode of the light-emitting device may be electrically connected to the source electrode or the drain electrode of the thin-film transistor. In some embodiments, the electronic apparatus may further include a color filter, a color conversion layer, a touch screen layer, a polarizing layer, or any combination thereof. For more details on the electronic apparatus, related descriptions provided herein may be referred to.

One or more aspects of embodiments of the present disclosure are directed toward electronic equipment including the electronic apparatus, and the electronic equipment may be at least one of a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented reality display, a vehicle, a video wall including multiple displays tiled together, a theater or stadium screen, a phototherapy device, or a signboard.

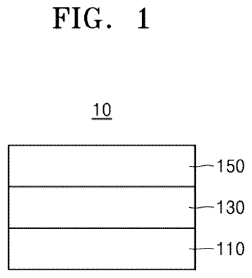

### Description of FIG. 1

FIG. 1 is a schematic cross-sectional view of a light-emitting device 10 according to one or more embodiments of the present disclosure. The light-emitting device 10 may include a first electrode 110, an interlayer 130, and a second electrode 150.

Hereinafter, the structure of the light-emitting device 10 according to one or more embodiments and a method of manufacturing the light-emitting device 10 will be described with reference to FIG. 1.

### First Electrode 110

In FIG. 1, in one or more embodiments, a substrate may be additionally provided and located under the first electrode 110 and/or on the second electrode 150. As the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (AI), aluminum-lithium (Al—Li), calcium (Ca), magnesium-indium (Mg—In), magnesium-silver (Mg—Ag), or any combination thereof.

The first electrode 110 may have a single-layered structure including (e.g., consisting of) a single layer or a multi-layered structure including a plurality of layers.

For example, in some embodiments, the first electrode 110 may have a three-layered structure of ITO/Ag/ITO.

### Interlayer 130

The interlayer 130 may be located on the first electrode 110. The interlayer 130 may include an emission layer.

In one or more embodiments, the interlayer 130 may further include a hole transport region between the first electrode 110 and the emission layer, and an electron transport region between the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer between the two or more emitting units. When the interlayer 130 includes emitting units and a charge generation layer as described above, the light-emitting device 10 may be a tandem light-emitting device.

### Hole Transport Region in Interlayer 130

The hole transport region may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The hole transport region may include a hole injection layer, a hole transport layer, an emission auxiliary layer, an electron-blocking layer, or any combination thereof.

For example, in one or more embodiments, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the constituent layers of each structure being stacked sequentially from the first electrode 110 in the stated order.

In one or more embodiments, the hole transport region may include a compound represented by Formula 201, a compound represented by Formula 202, or any combination thereof:

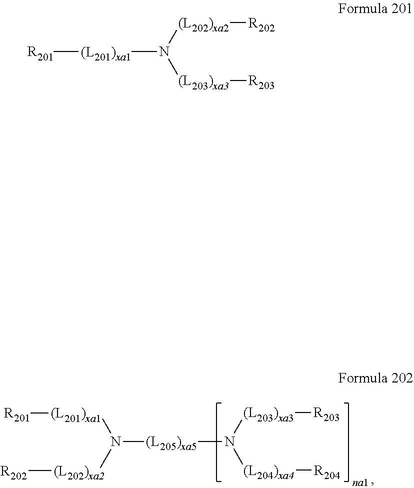

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, see Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, in some embodiments, each of Formulae 201 and 202 may include at least one selected from groups represented by Formulae CY201 to CY217:

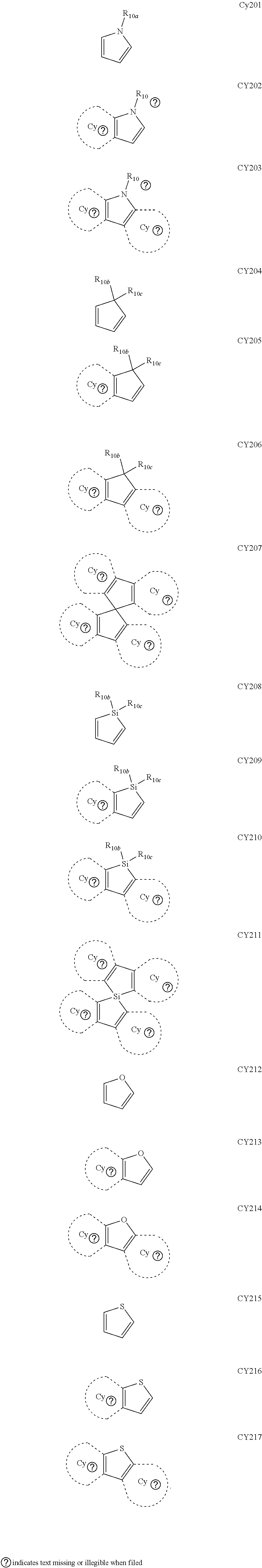

In Formulae CY201 to CY217, R_{10b} and R_{10c} may each be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include (e.g., may exclude) any group represented by one selected from Formulae CY201 to CY203.

In one or more embodiments, each of Formulae 201 and 202 may not include (e.g., may exclude) any group represented by one selected from Formulae CY201 to CY203, and may include at least one selected from the groups represented by Formulae CY204 to CY217.

In one or more embodiments, each of Formulae 201 and 202 may not include (e.g., may exclude) any group represented by one selected from Formulae CY201 to CY217. In present disclosure, “not include a or any ‘component’” “exclude a or any ‘component’”, “‘component’-free”, and/or the like refers to that the “component” not being added, selected or utilized as a component in the composition/formula/element, but, in some embodiments, the “component” of less than a suitable amount may still be included due to other impurities and/or external factors.

In one or more embodiments, the hole transport region may include at least one selected from among Compounds HT1 to HT46, 4,4′, 4″-[tris(3-methylphenyl)phenylamino]triphenylamine (m-MTDATA), 4,4′, 4″-tris(N,N-diphenylamino)triphenylamine (TDATA), 4,4′, 4″-tris[N-(2-naphthyl)-N-phenylamino]-triphenylamine (2-TNATA), N,N′-di(naphthalen-1-yl)-N,N′-diphenyl-benzidine (NPB(NPD)), β-NPB, N,N′-bis(3-methylphenyl)-N,N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′, 4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

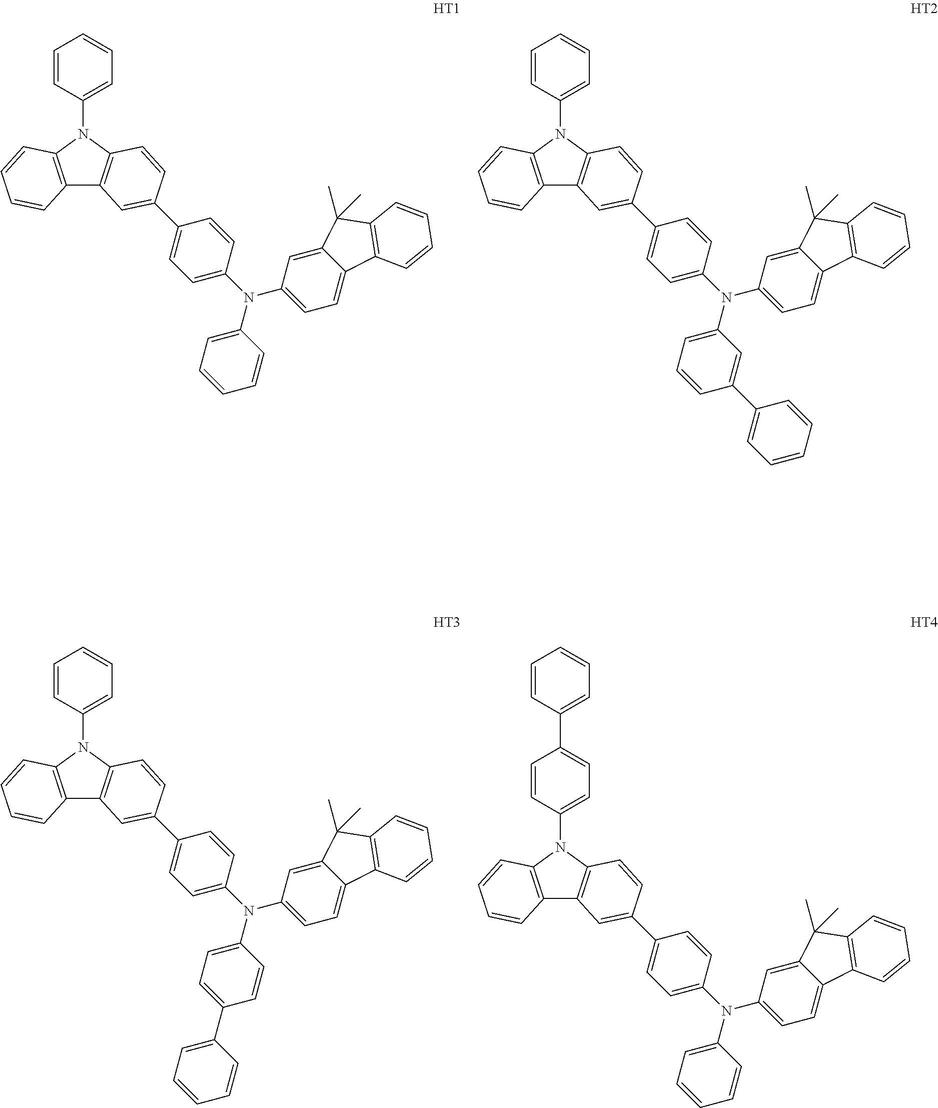 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

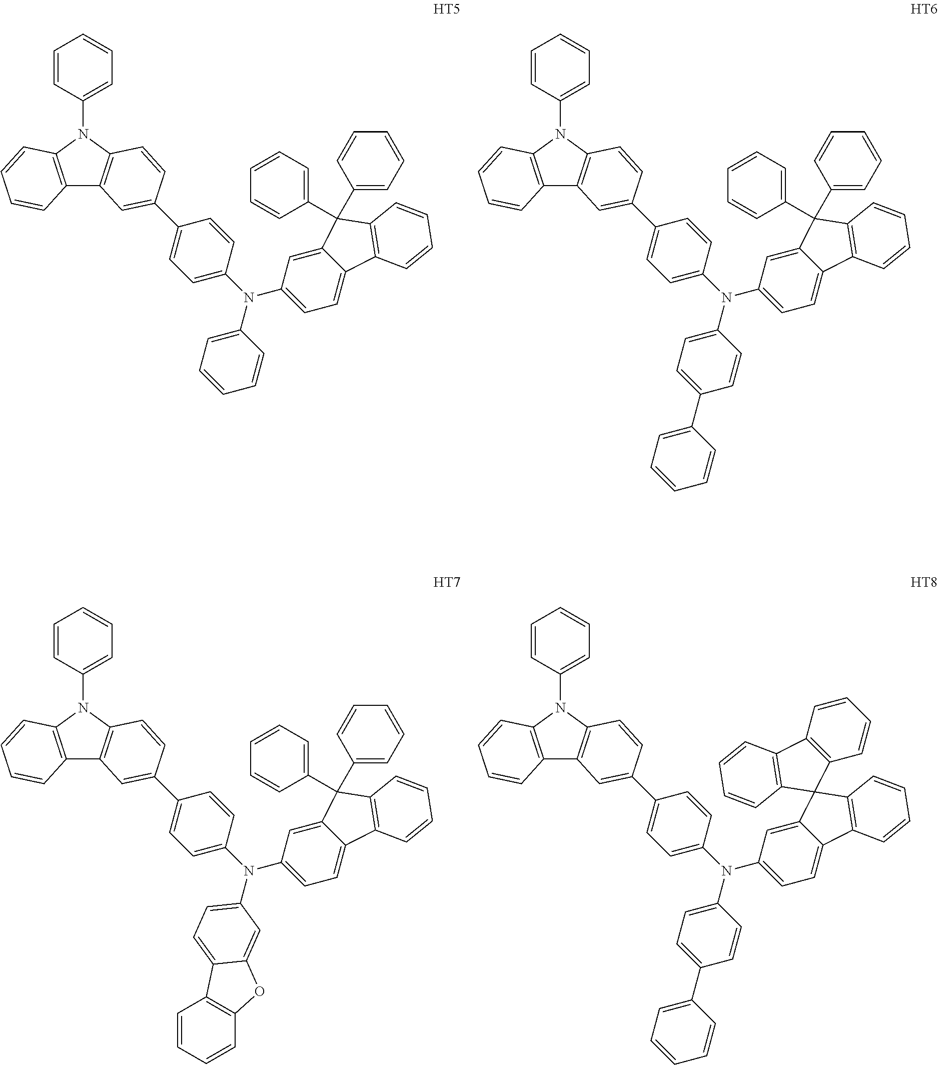 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

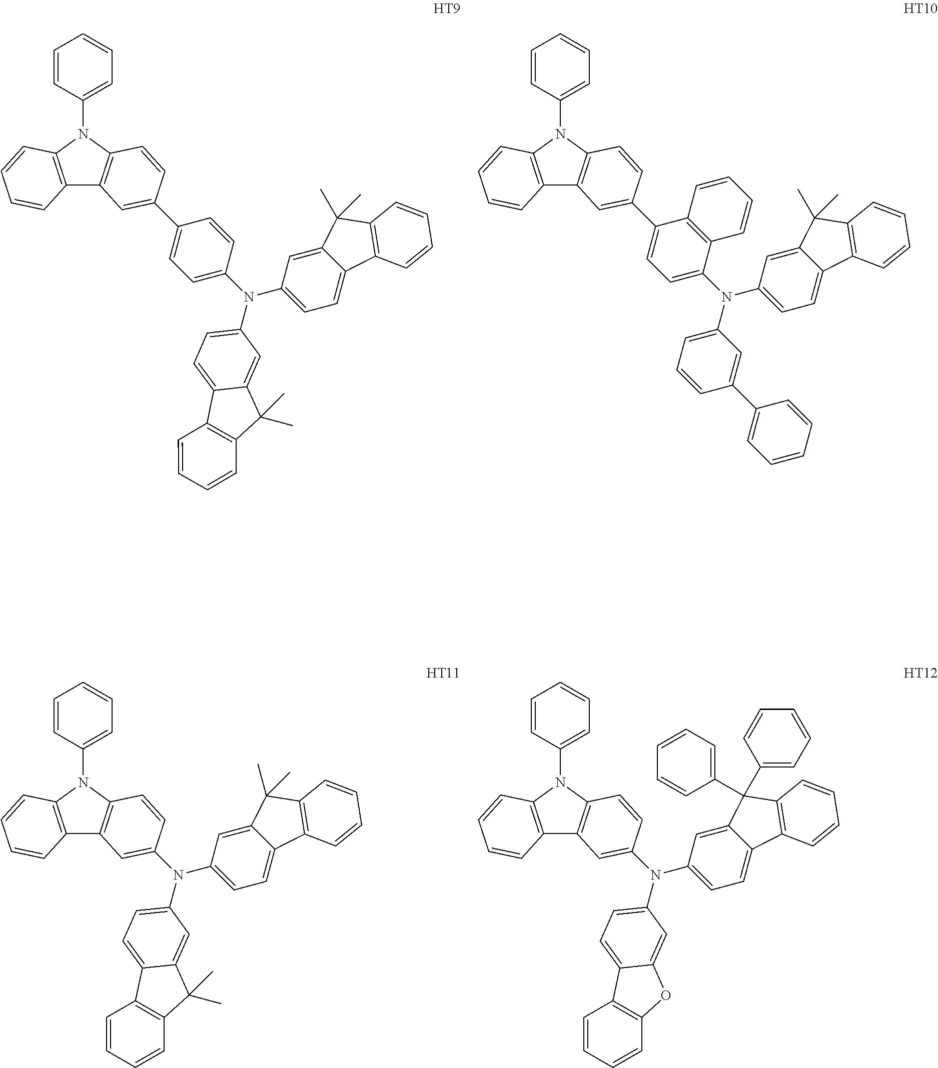 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

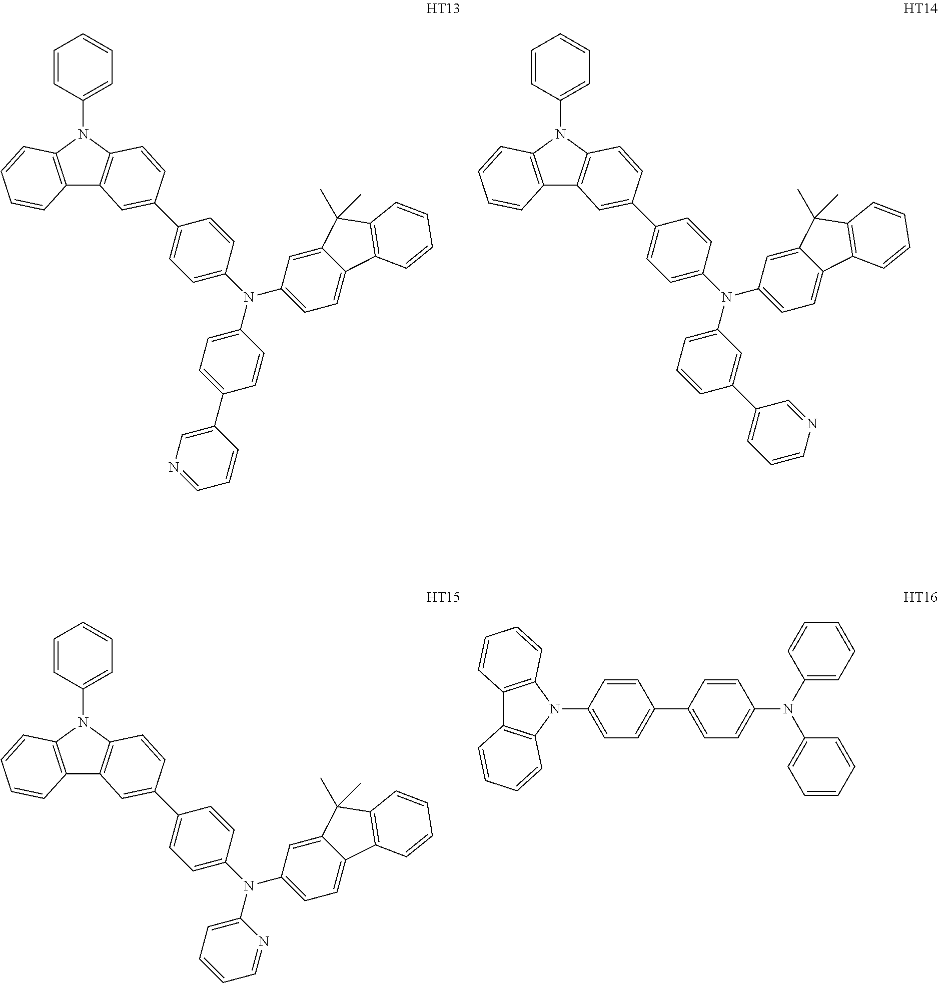 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

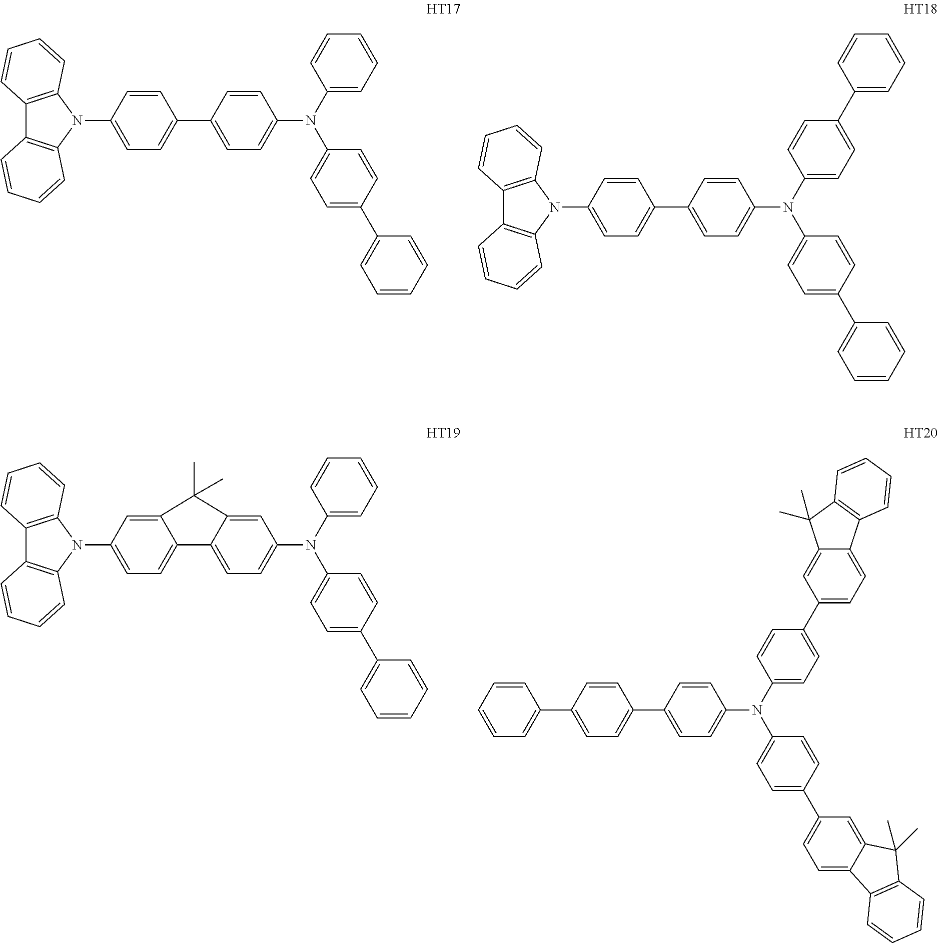 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

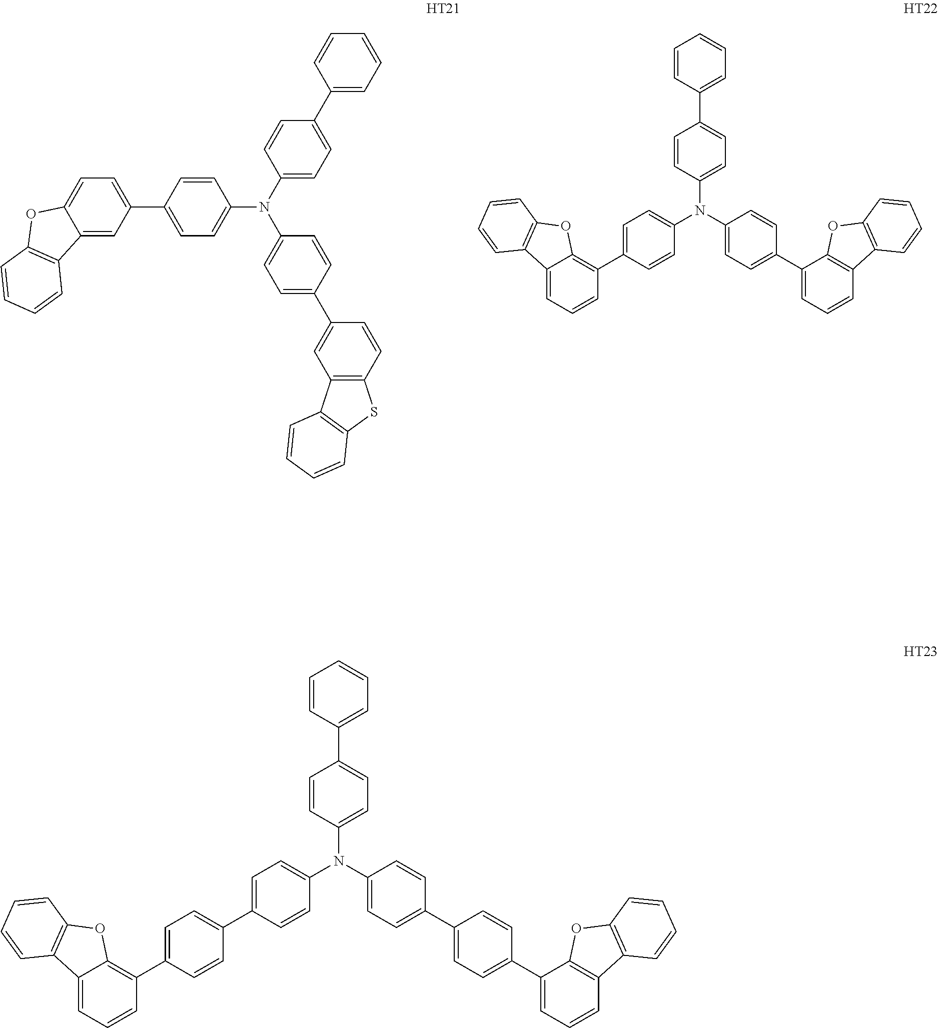 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

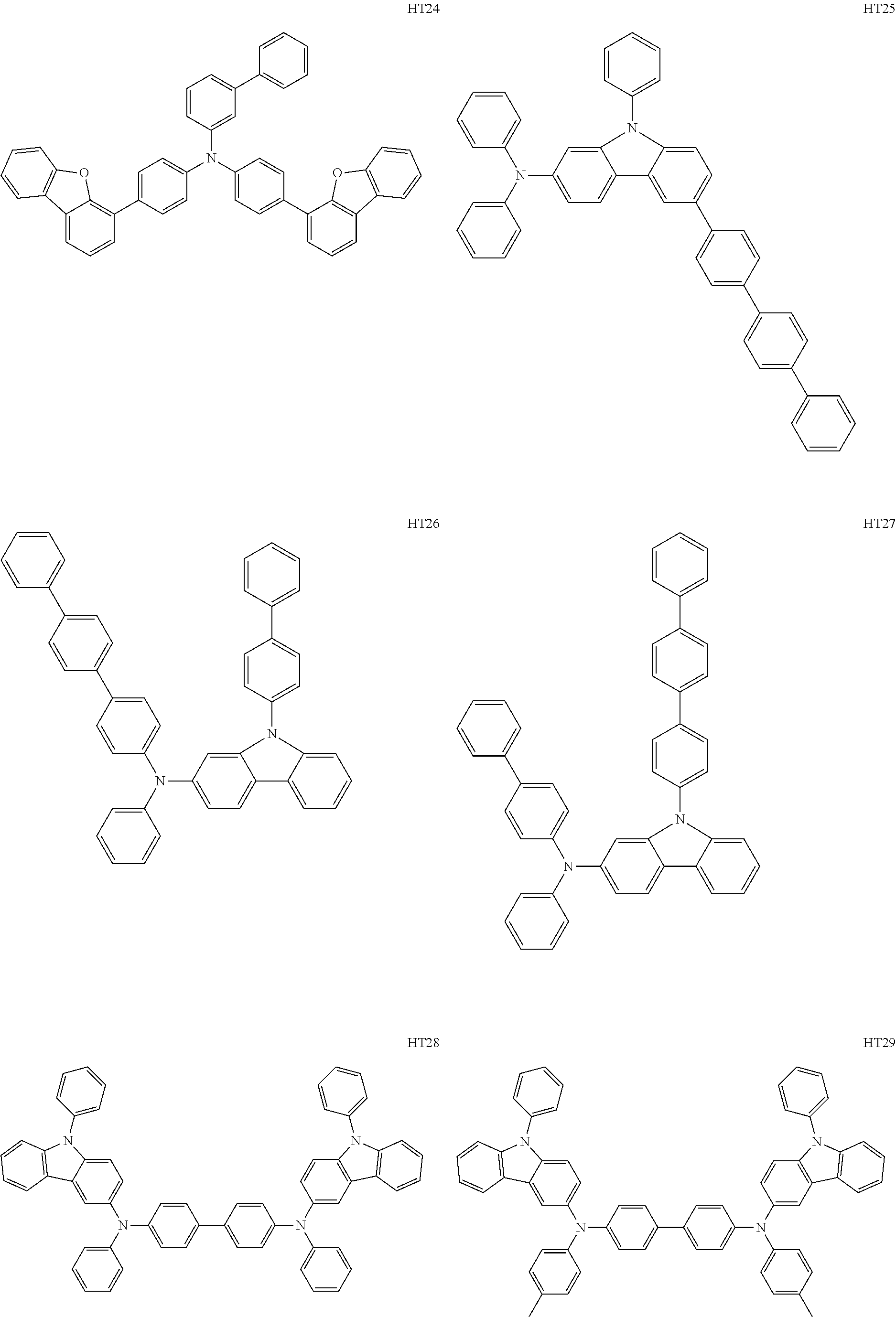

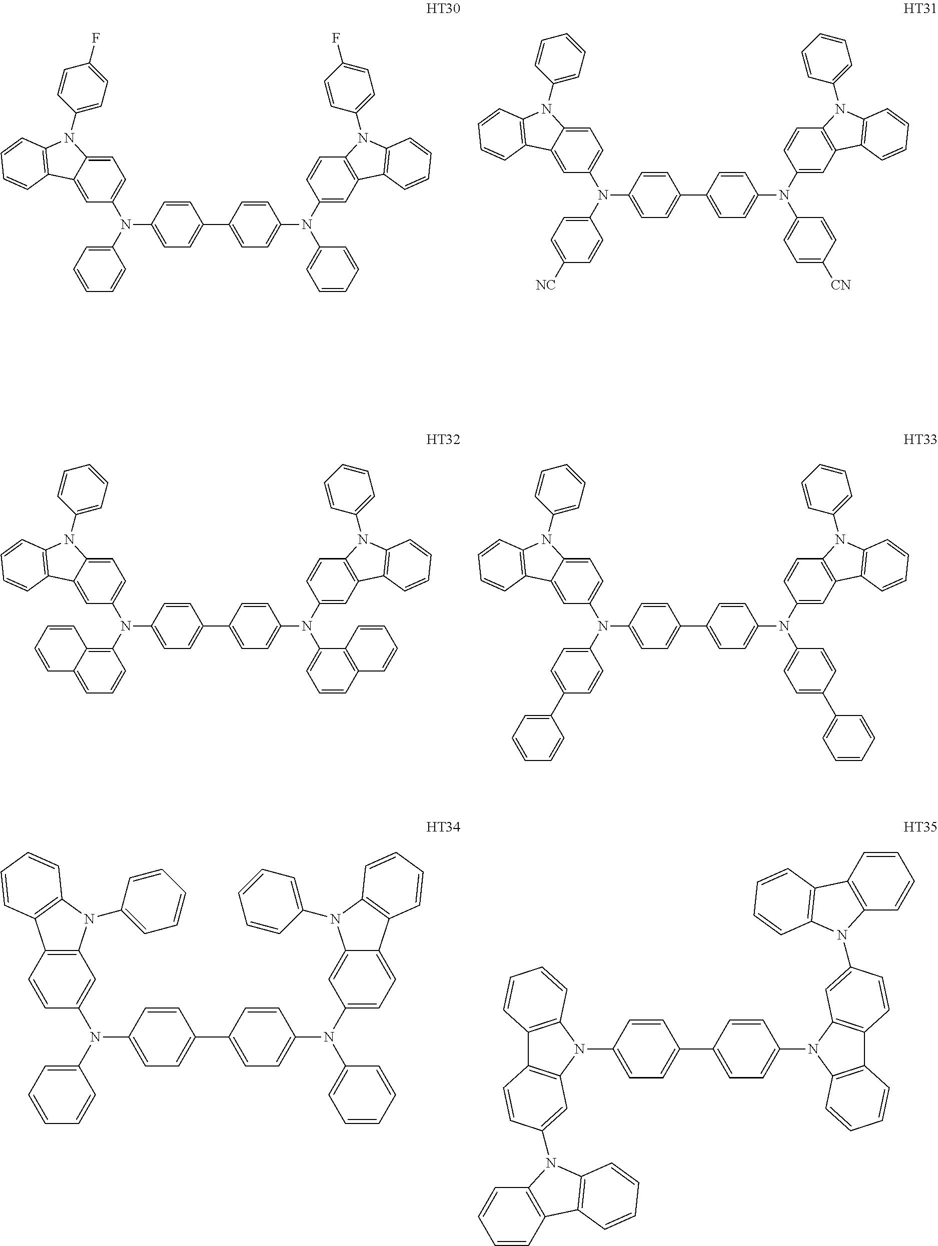

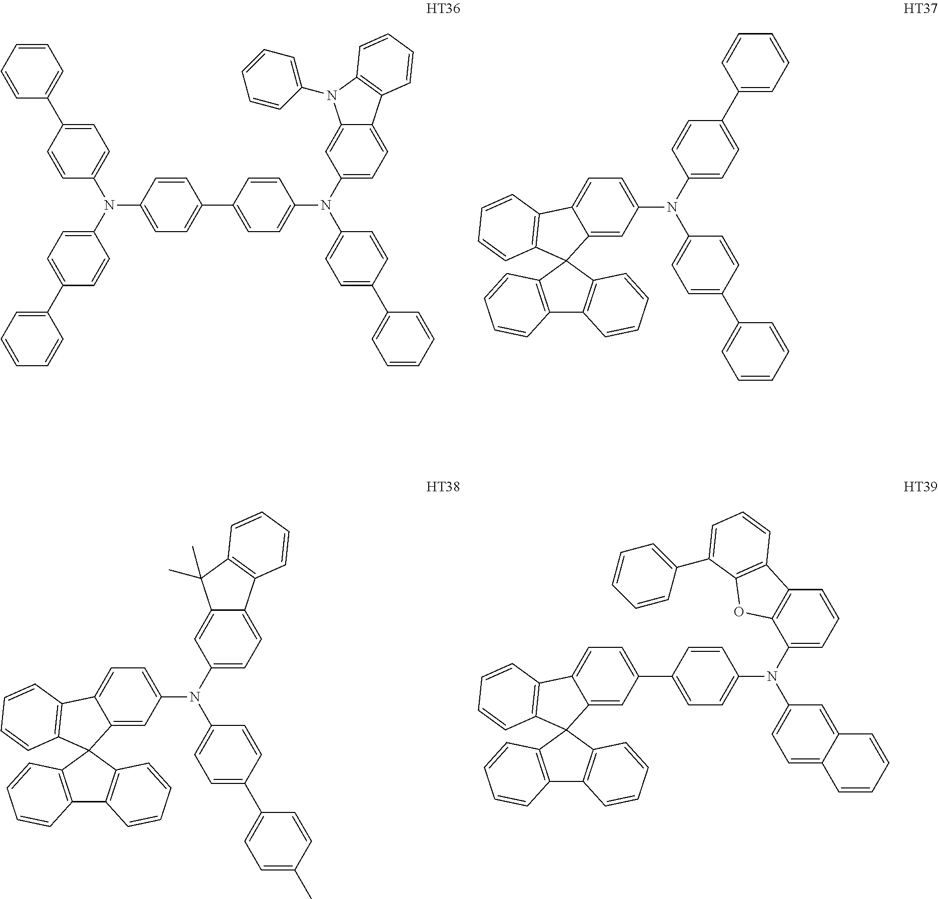 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

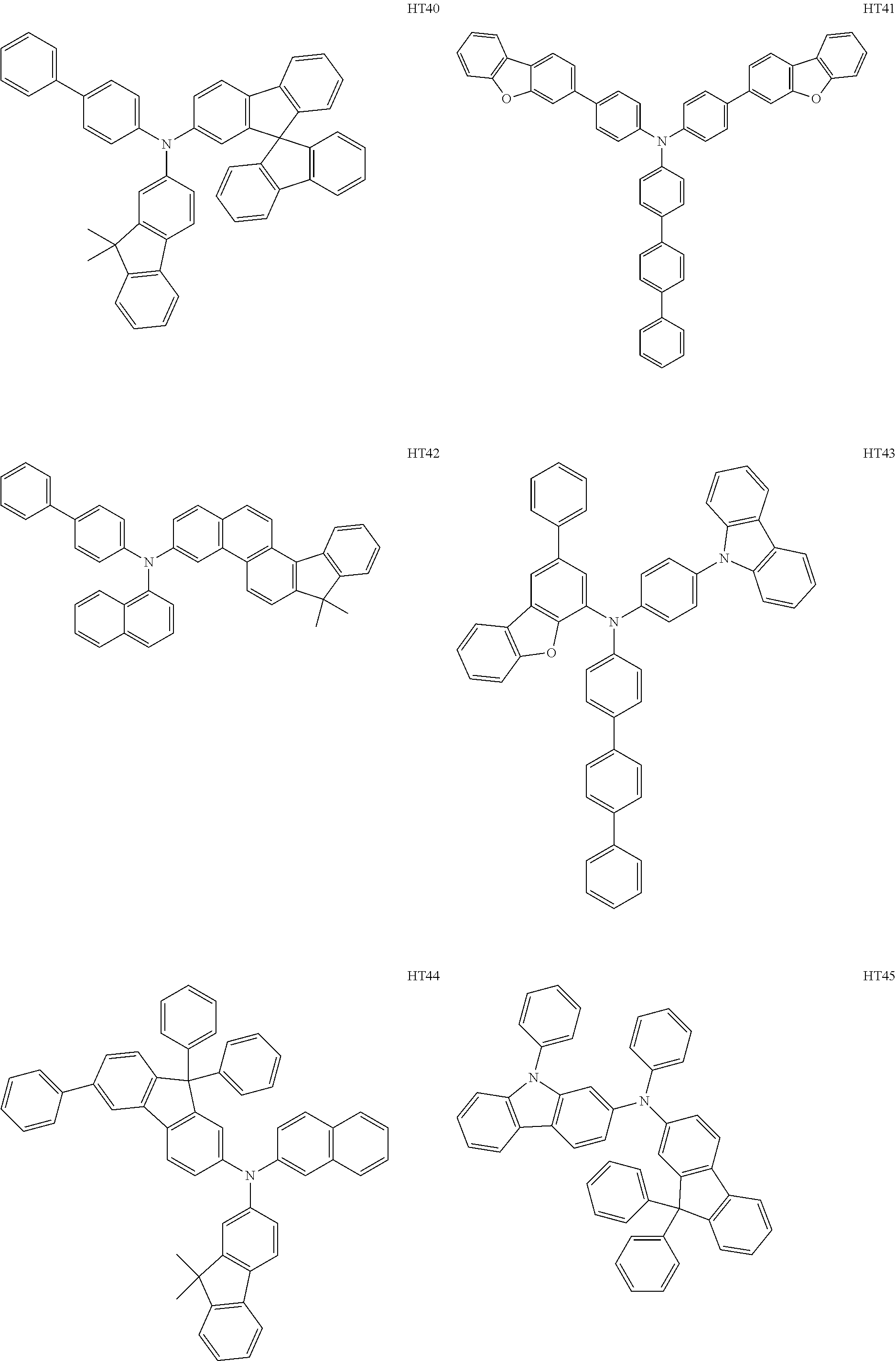

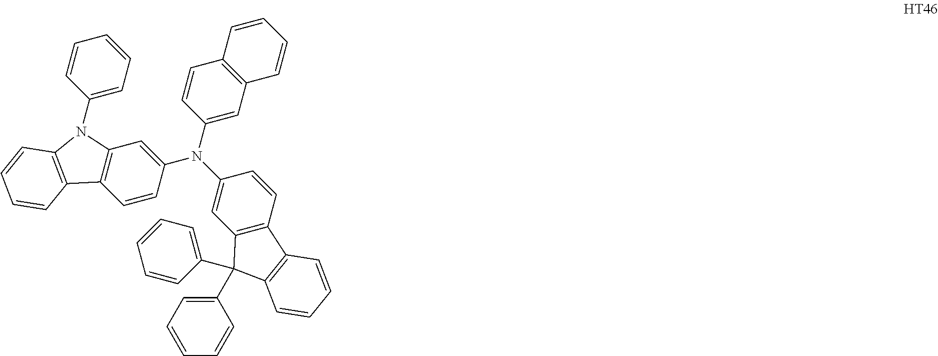 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

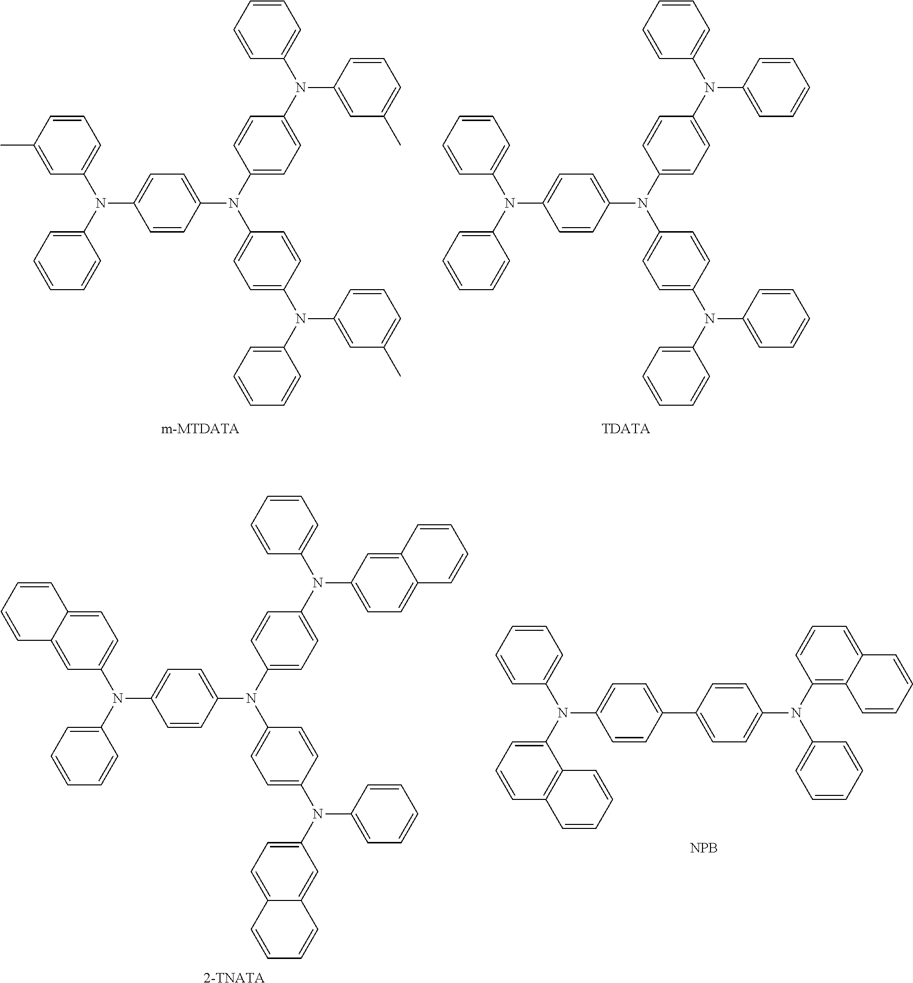

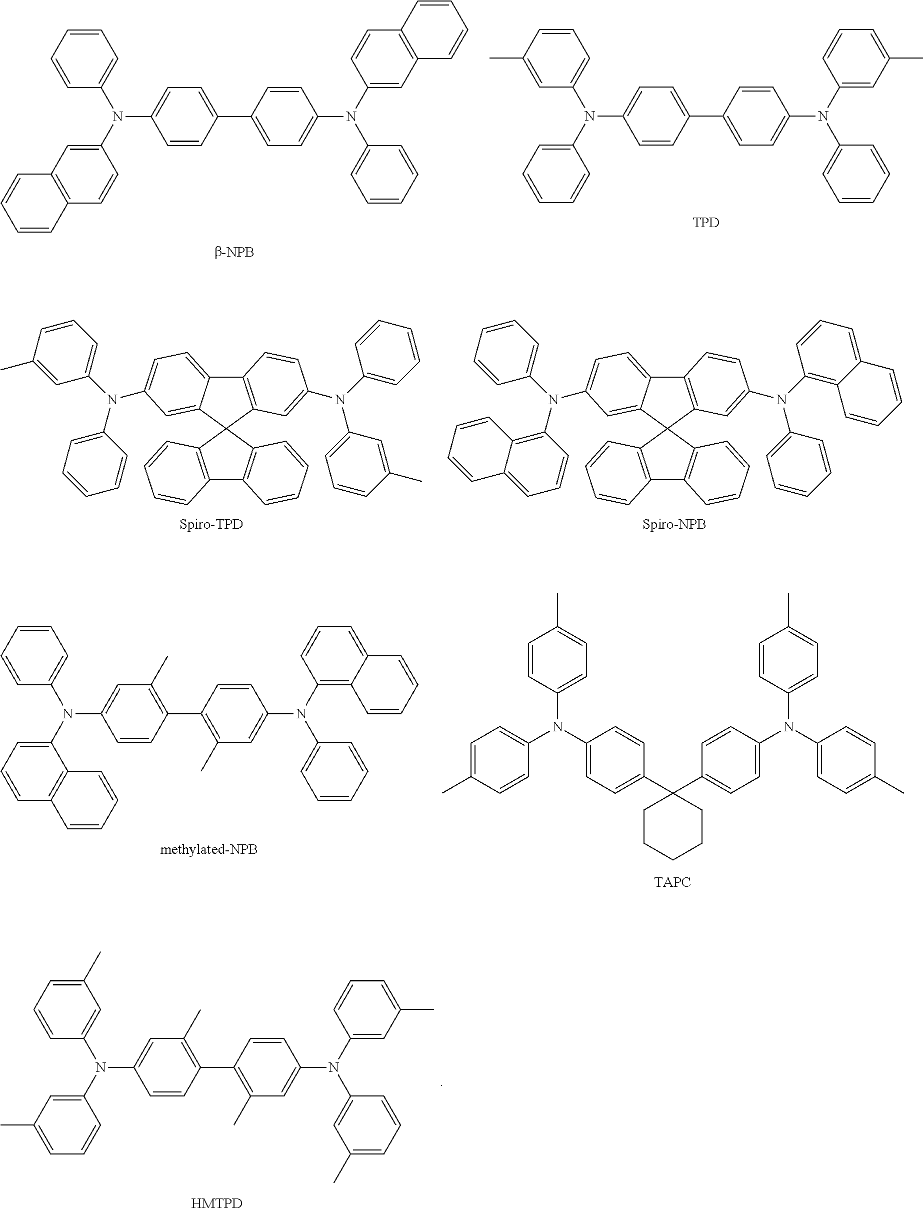

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron-blocking layer may block or reduce the leakage of electrons from the emission layer to the hole transport region. Materials that may be included in the hole transport region may be included in the emission auxiliary layer and the electron-blocking layer.

p-dopant

In one or more embodiments, the hole transport region may further include, in addition to the aforementioned materials, a charge-generation material for the improvement of conductive properties. The charge-generation material may be substantially uniformly or non-uniformly dispersed in the hole transport region (for example, in the form of a single layer including (e.g., consisting of) a charge-generation material).

The charge-generation material may be, for example, a p-dopant.

For example, in one or more embodiments, a lowest unoccupied molecular orbital (LUMO) energy level of the p-dopant may be −3.5 eV or less.

In one or more embodiments, the p-dopant may include a quinone derivative, a cyano group-containing compound, a compound including element EL1 and element EL2, or any combination thereof.

Non-limiting examples of the quinone derivative are tetracyanoquinodimethane (TCNQ), 2,3,5,6-tetrafluoro-7,7,8,8-tetracyanoquinodimethane (F4-TCNQ), etc.

Non-limiting examples of the cyano group-containing compound are dipyrazino[2,3-f: 2′, 3′-h]quinoxaline-2,3,6,7,10,11-hexacarbonitrile (HAT-CN), and a compound represented by Formula 221:

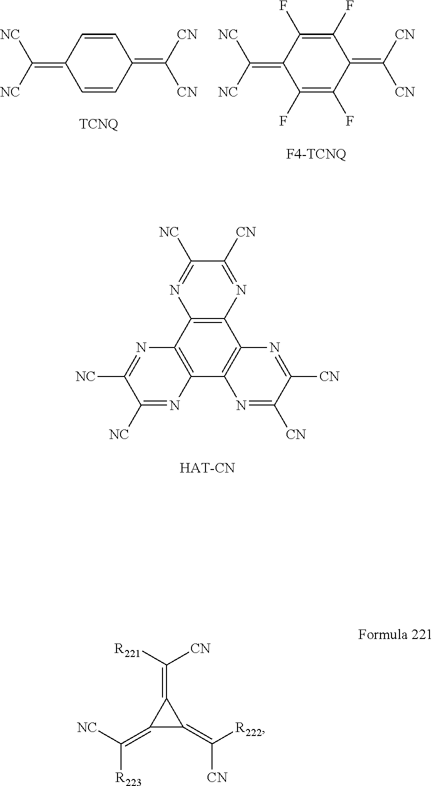

wherein, in Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from among R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Non-limiting examples of the metal may be an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Non-limiting examples of the metalloid may be silicon (Si), antimony (Sb), and/or tellurium (Te).

Non-limiting examples of the non-metal may be oxygen (O) and/or a halogen (for example, F, Cl, Br, I, etc.).

Non-limiting examples of the compound including element EL1 and element EL2 may be metal oxides, metal halides (for example, metal fluorides, metal chlorides, metal bromides, or metal iodides), metalloid halides (for example, metalloid fluorides, metalloid chlorides, metalloid bromides, or metalloid iodides), metal tellurides, or one or more combinations thereof.

Non-limiting examples of the metal oxide may be tungsten oxides (for example, WO, W₂O₃, WO₂, WO₃, W₂O₅, etc.), vanadium oxides (for example, VO, V₂O₃, VO₂, V₂O₅, etc.), molybdenum oxides (MoO, Mo₂O₃, MoO₂, MoO₃, Mo₂O₅, etc.), and/or rhenium oxides (for example, ReO₃, etc.).

Non-limiting examples of the metal halide may be alkali metal halides, alkaline earth metal halides, transition metal halides, post-transition metal halides, and/or lanthanide metal halides.

Non-limiting examples of the alkali metal halide may be LiF, NaF, KF, RbF, CsF, LiCl, NaCl, KCl, RbCI, CsCl, LiBr, NaBr, KBr, RbBr, CsBr, LiI, NaI, KI, RbI, CsI, and/or the like.

Non-limiting examples of the alkaline earth metal halide may be BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and/or BaI2.

Non-limiting examples of the transition metal halide may be titanium halides (for example, TiF₄, TiCl₄, TiBr₄, TiI4, etc.), zirconium halides (for example, ZrF₄, ZrCl₄, ZrBr₄, ZrI₄, etc.), hafnium halides (for example, HfF₄, HfCl₄, HfBr₄, HfI₄, etc.), vanadium halides (for example, VF₃, VCl₃, VBr₃, VI₃, etc.), niobium halides (for example, NbF₃, NbCl₃, NbBr₃, NbI₃, etc.), tantalum halides (for example, TaF₃, TaCl₃, TaBr₃, TaI₃, etc.), chromium halides (for example, CrF₃, CrO₃, CrBr₃, CrI₃, etc.), molybdenum halides (for example, MoF₃, MoCl₃, MoBr₃, MoI₃, etc.), tungsten halides (for example, WF₃, WCl₃, WBr₃, WI₃, etc.), manganese halides (for example, MnF₂, MnCl₂, MnBr₂, MnI₂, etc.), technetium halides (for example, TcF₂, TcCl₂, TcBr₂, TcI₂, etc.), rhenium halides (for example, ReF₂, ReCl₂, ReBr₂, ReI₂, etc.), ferrous halides (for example, FeF₂, FeCl₂, FeBr₂, FeI₂, etc.), ruthenium halides (for example, RuF₂, RuCl₂, RuBr₂, RuI₂, etc.), osmium halides (for example, OsF₂, OsCl₂, OsBr₂, OsI₂, etc.), cobalt halides (for example, CoF₂, COCl₂, CoBr₂, CoI₂, etc.), rhodium halides (for example, RhF₂, RhCl₂, RhBr₂, RhI₂, etc.), iridium halides (for example, IrF₂, IrCl₂, IrBr₂, IrI₂, etc.), nickel halides (for example, NiF₂, NiCl₂, NiBr₂, NiI₂, etc.), palladium halides (for example, PdF₂, PdCl₂, PdBr₂, PdI₂, etc.), platinum halides (for example, PtF₂, PtCl₂, PtBr₂, PtI₂, etc.), cuprous halides (for example, CuF, CuCl, CuBr, CuI, etc.), silver halides (for example, AgF, AgCl, AgBr, AgI, etc.), and/or gold halides (for example, AuF, AuCl, AuBr, AuI, etc.).

Non-limiting examples of the post-transition metal halide may be zinc halides (for example, ZnF₂, ZnCl₂, ZnBr₂, ZnI₂, etc.), indium halides (for example, InI₃, etc.), and/or tin halides (for example, SnI₂, etc.).

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide may be antimony halides (for example, SbCl₅ and/or the like) and/or the like.

Non-limiting examples of the metal telluride may be alkali metal tellurides (for example, Li₂Te, Na₂Te, K₂Te, Rb₂Te, Cs₂Te, and/or the like), alkaline earth metal tellurides (for example, BeTe, MgTe, CaTe, SrTe, BaTe, and/or the like), transition metal tellurides (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), post-transition metal tellurides (for example, ZnTe, and/or the like), lanthanide metal tellurides (for example, LaTe, CeTe, PrTe, NdTe, PmTe, EuTe, GdTe, TbTe, DyTe, HoTe, ErTe, TmTe, YbTe, LuTe, and/or the like), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers selected from a red emission layer, a green emission layer, and a blue emission layer, in which the two or more layers contact each other or are separated from each other to emit white light (e.g., combined white light). In one or more embodiments, the emission layer may include two or more materials selected from a red light-emitting material, a green light-emitting material, and a blue light-emitting material, in which the two or more materials are mixed with each other in a single layer to emit white light (e.g., combined white light).

In one or more embodiments, the emission layer may include a host and a dopant. The dopant may include a phosphorescent dopant, a fluorescent dopant, or any combination thereof.

An amount of the dopant in the emission layer may be from about 0.01 part by weight to about 15 parts by weight based on 100 parts by weight of the host.

In one or more embodiments, the emission layer may include a quantum dot.

In some embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent or suitable light-emission characteristics may be obtained without a substantial increase in driving voltage.

### Host

In one or more embodiments, the host may further include a compound represented by Formula 301:

[Ar₃₀₁]_{xb11}-[(L₃₀₁)_{xb1}-R₃₀₁]_{xb21}. Formula 301

In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ are each the same as described herein with respect to Q₁.

For example, in some embodiments, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

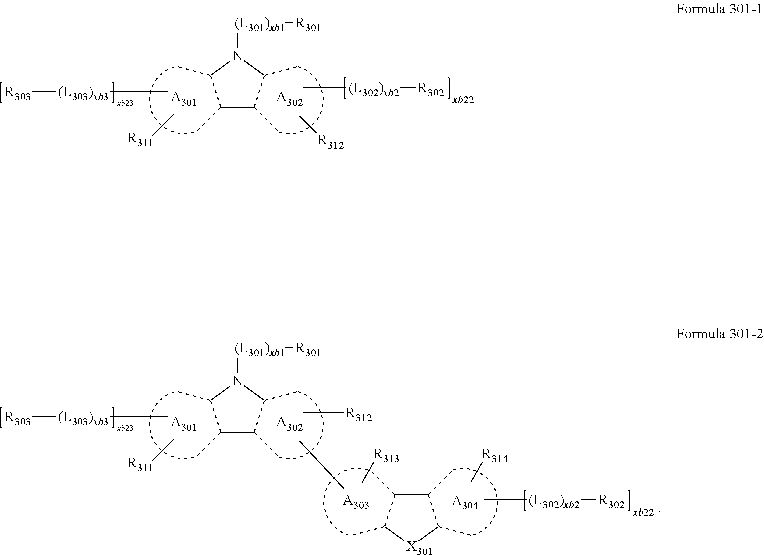

In Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each be the same as described herein,

L₃₀₂ to L₃₀₄ may each independently be the same as described herein with respect to with L₃₀₁,

xb2 to xb4 may each independently be the same as described herein with respect to xb1, and

R₃₀₂ to R₃₀₅ and R₃₁₁ to R₃₁₄ may each be the same as described herein with respect to R₃₀₁.

In one or more embodiments, the host may include an alkaline earth metal complex, a post-transition metal complex, or any combination thereof. For example, the host may include a Be complex (for example, Compound H55), an Mg complex, a Zn complex, or any combination thereof.

In one or more embodiments, the host may include: at least one selected from among Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(9H-carbazol-9-yl)benzene (mCP); 1,3,5-tri(carbazol-9-yl)benzene (TCP); or any combination thereof:

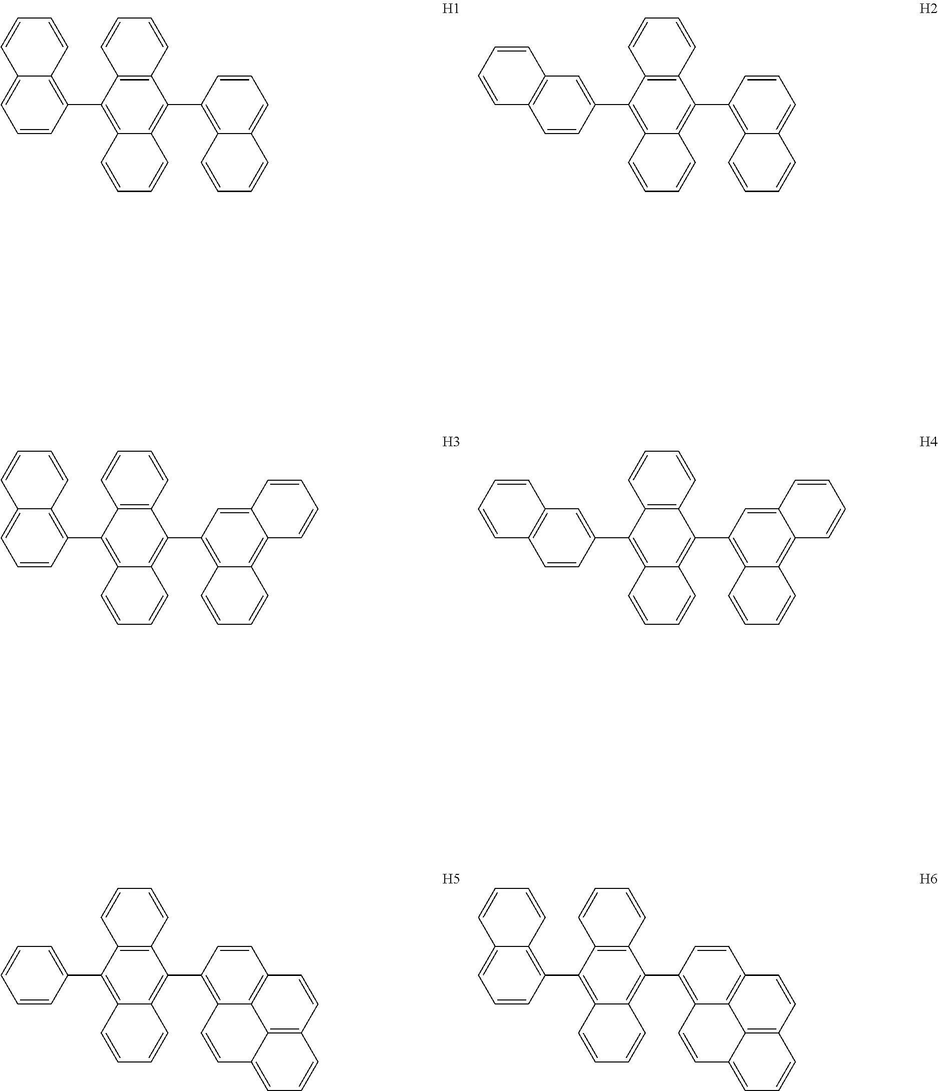

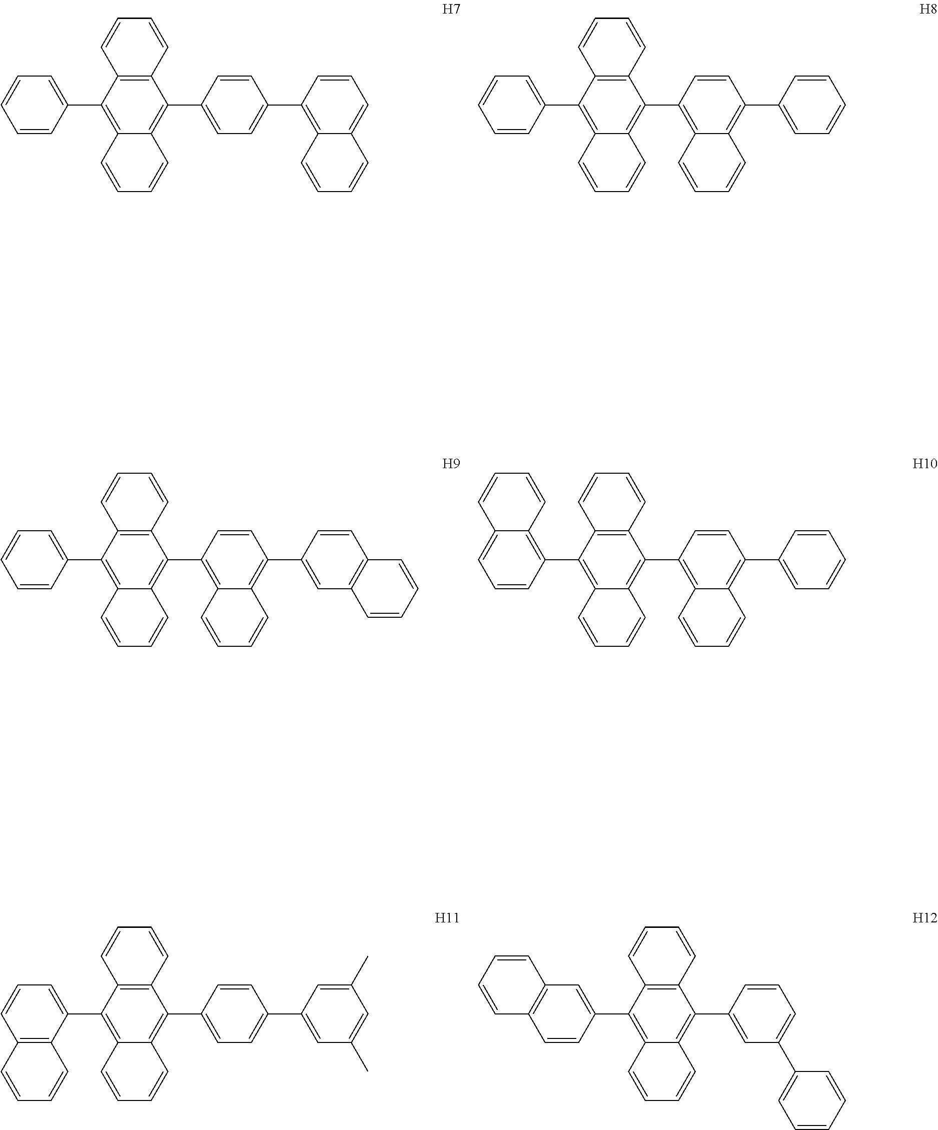

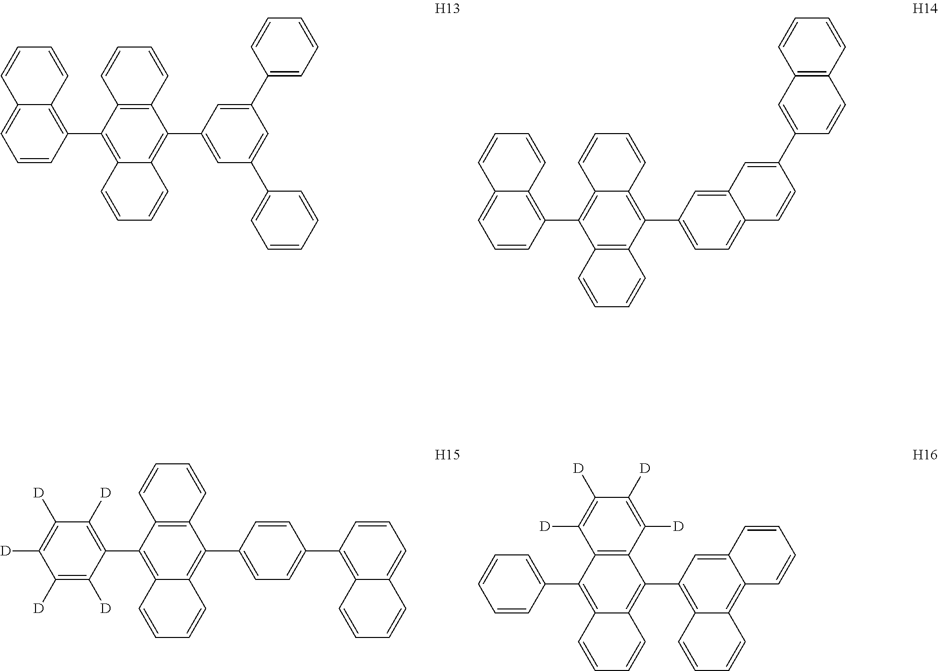

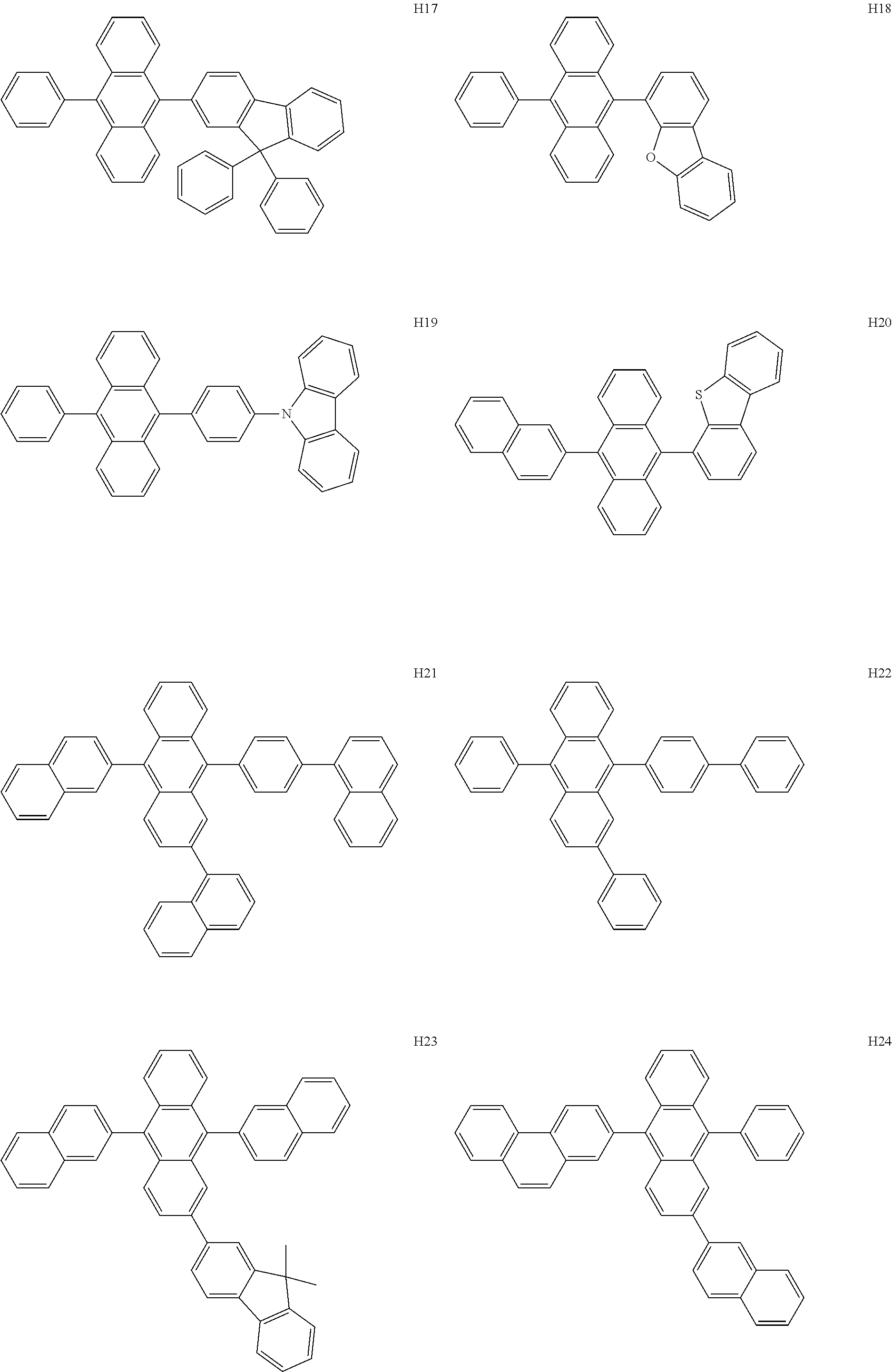

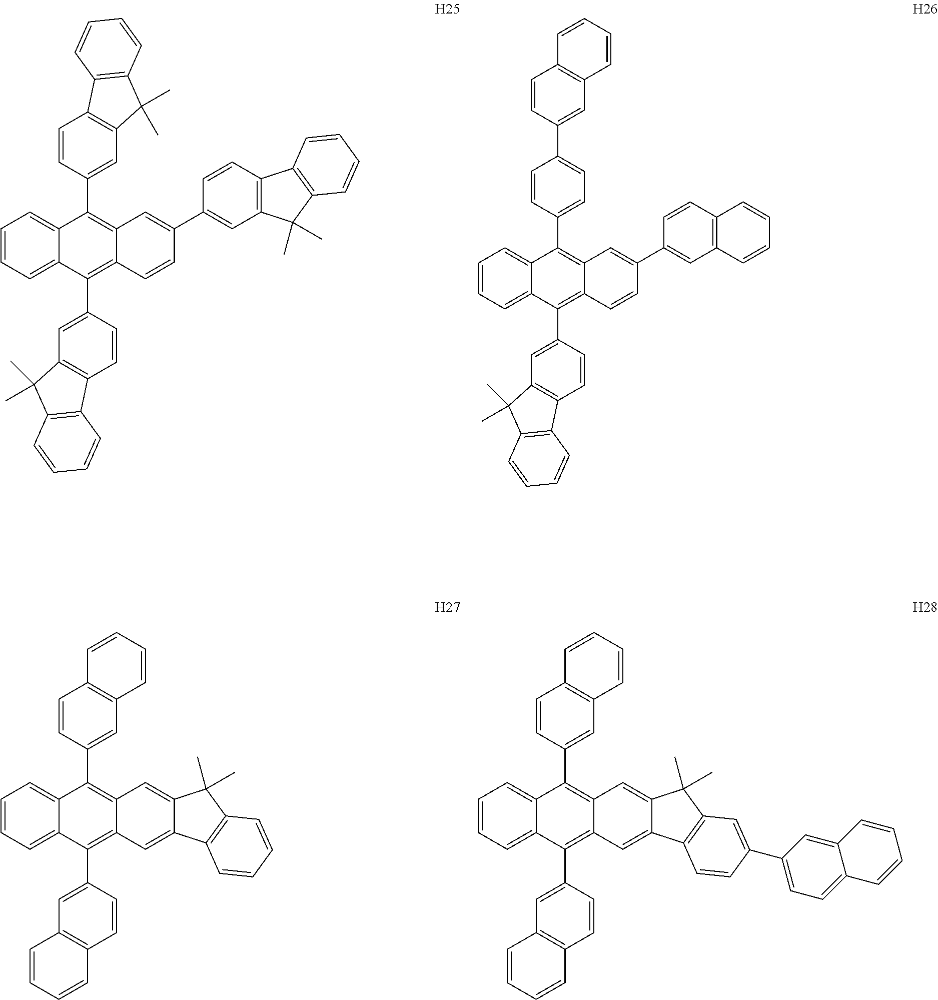

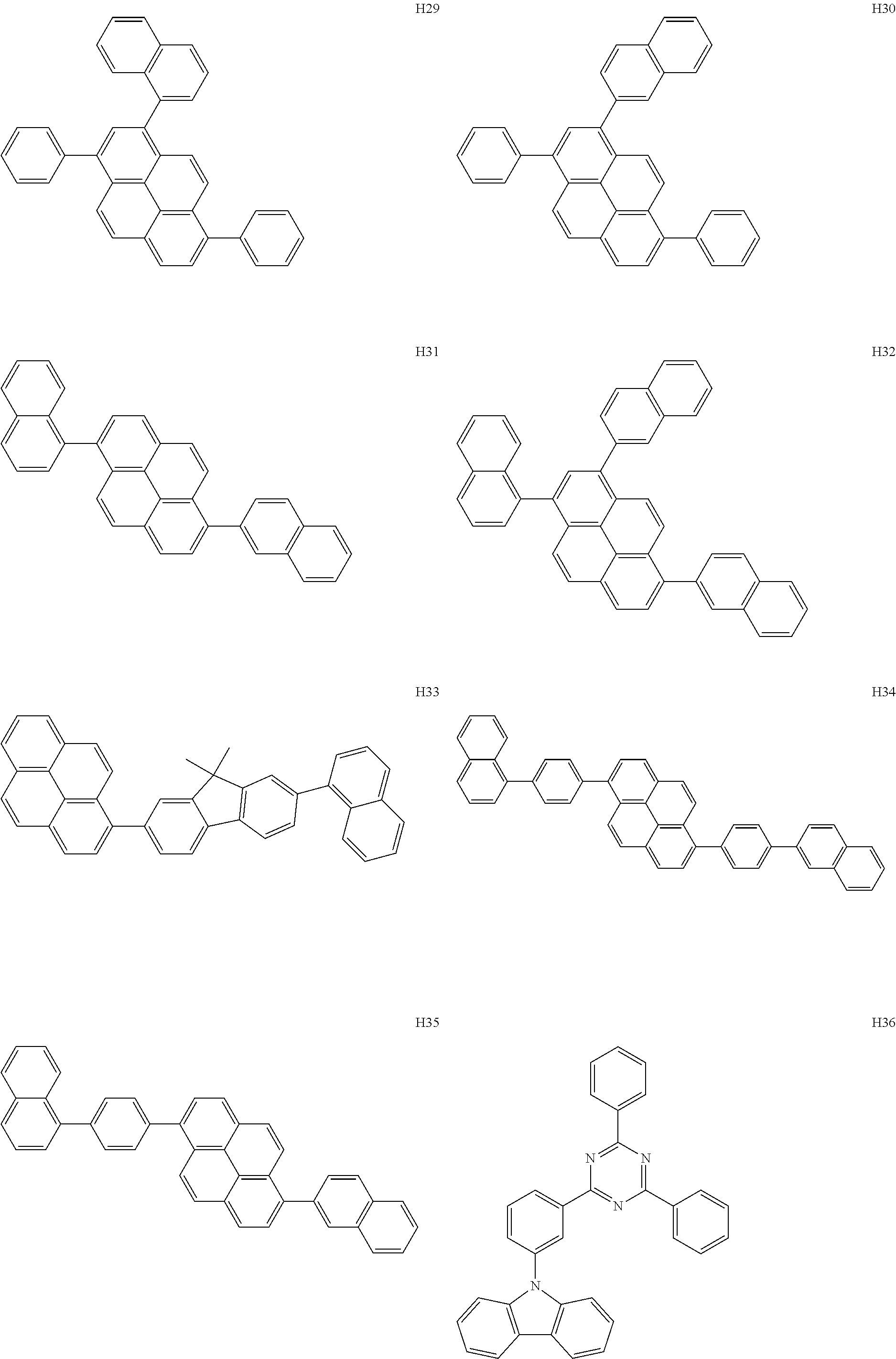

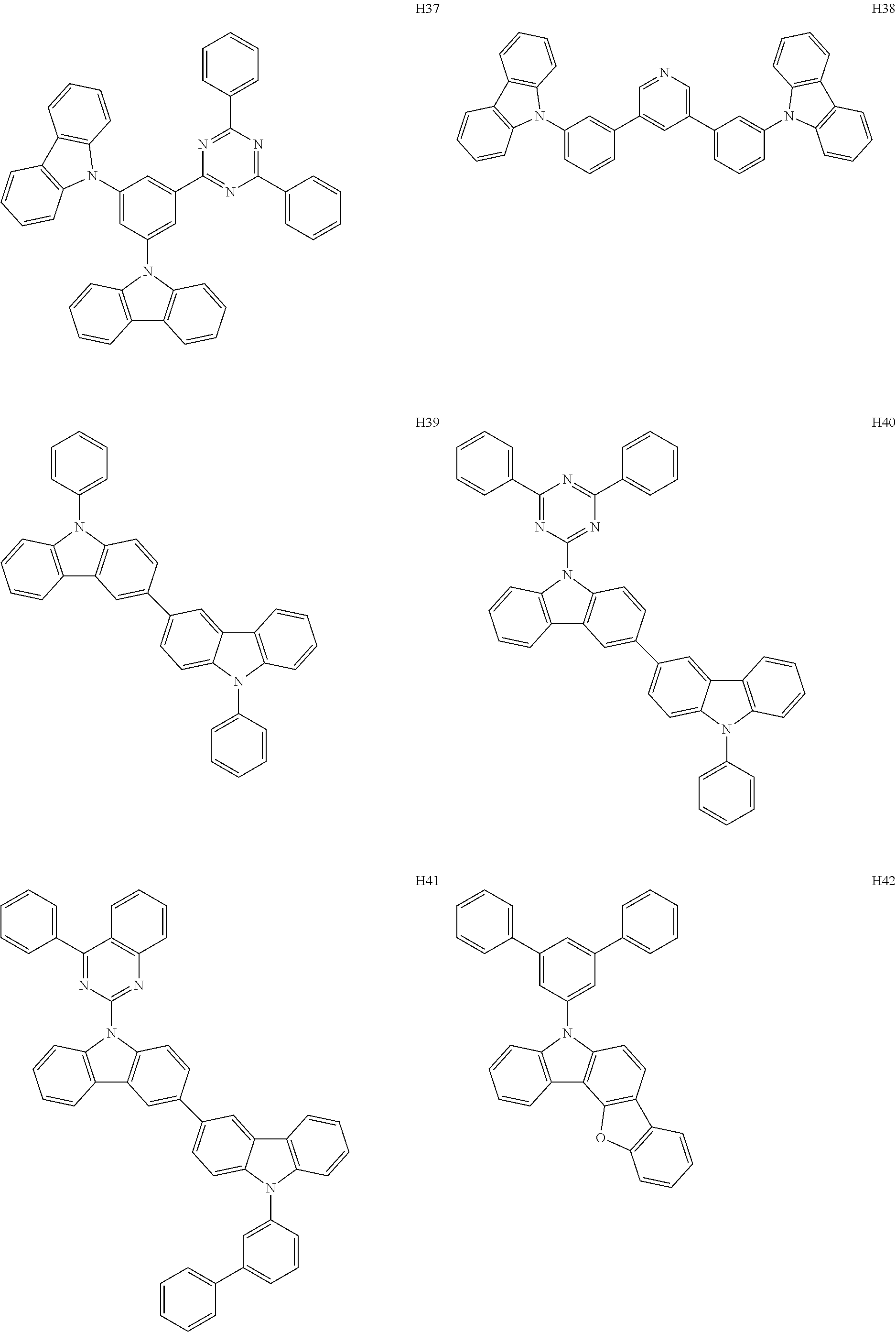 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

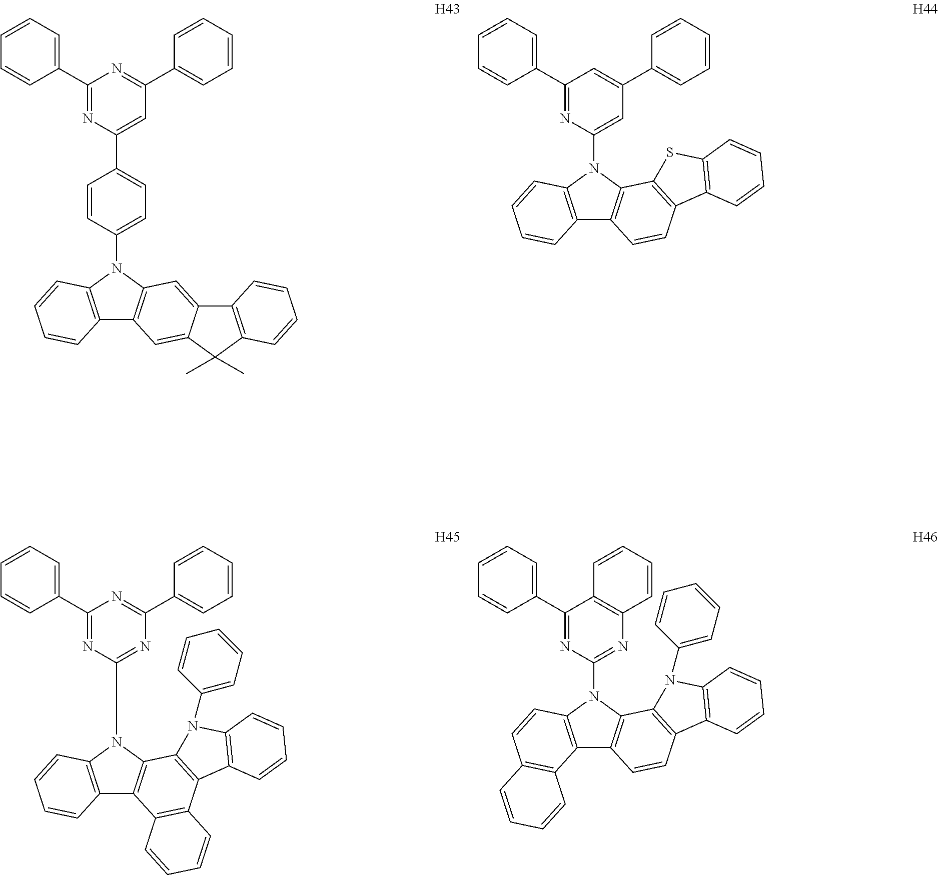 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

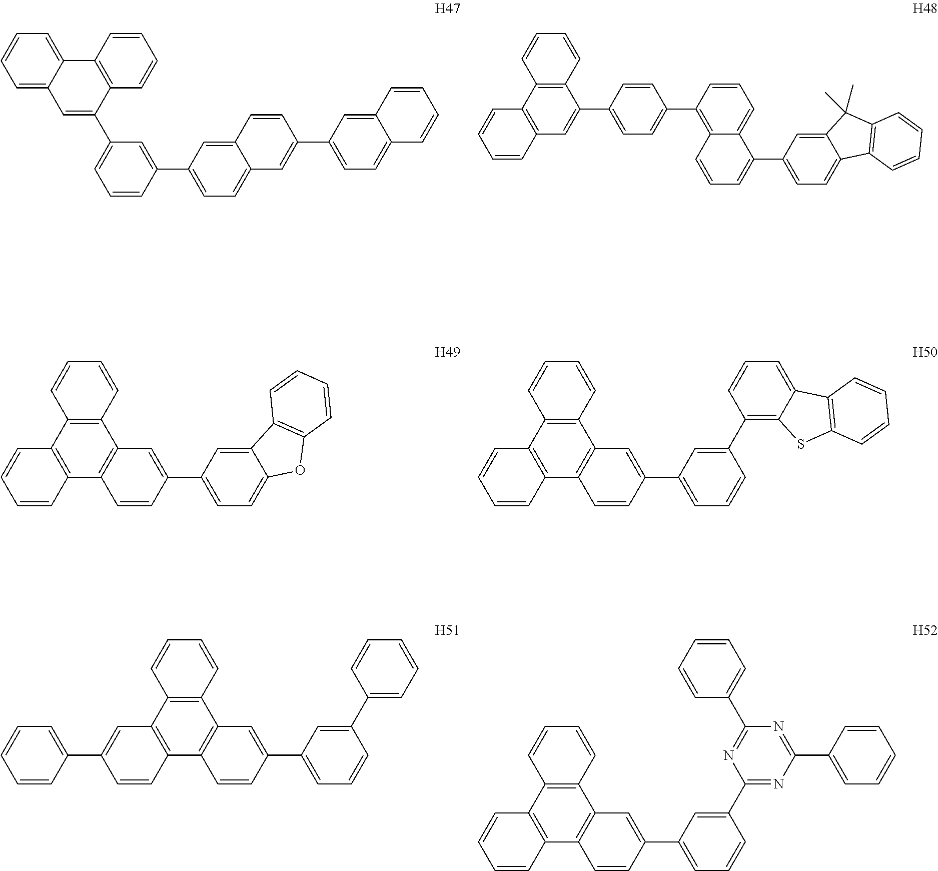

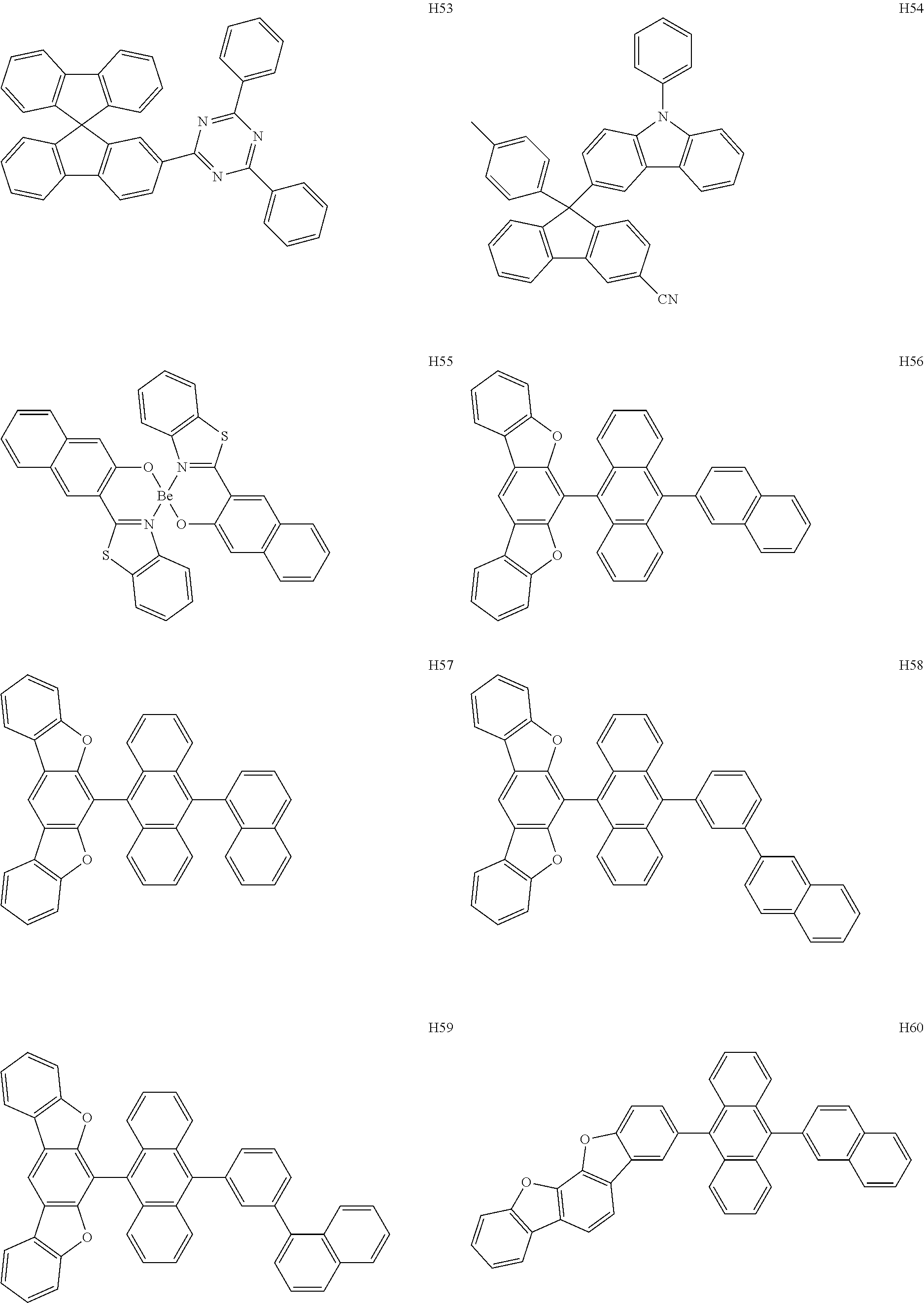

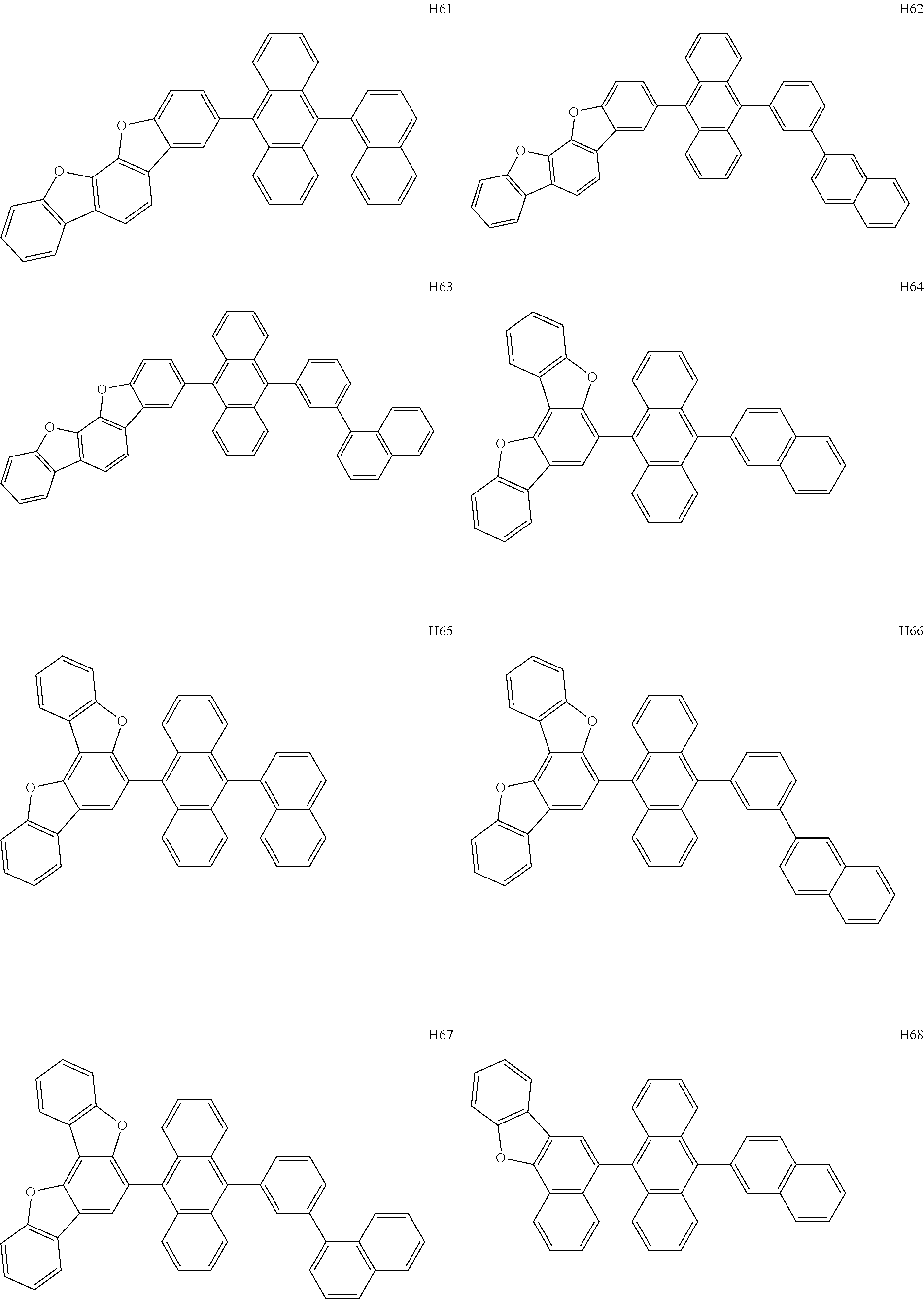

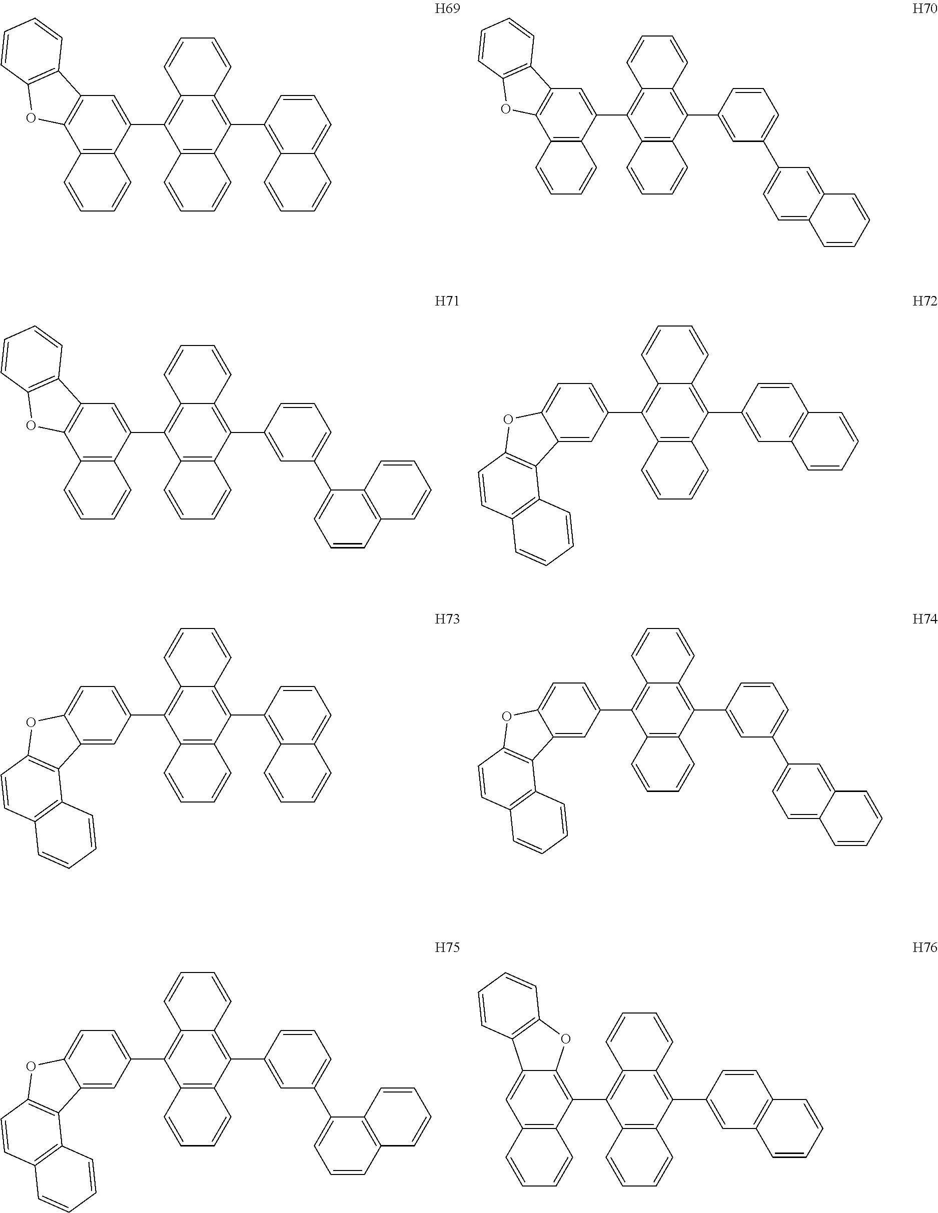

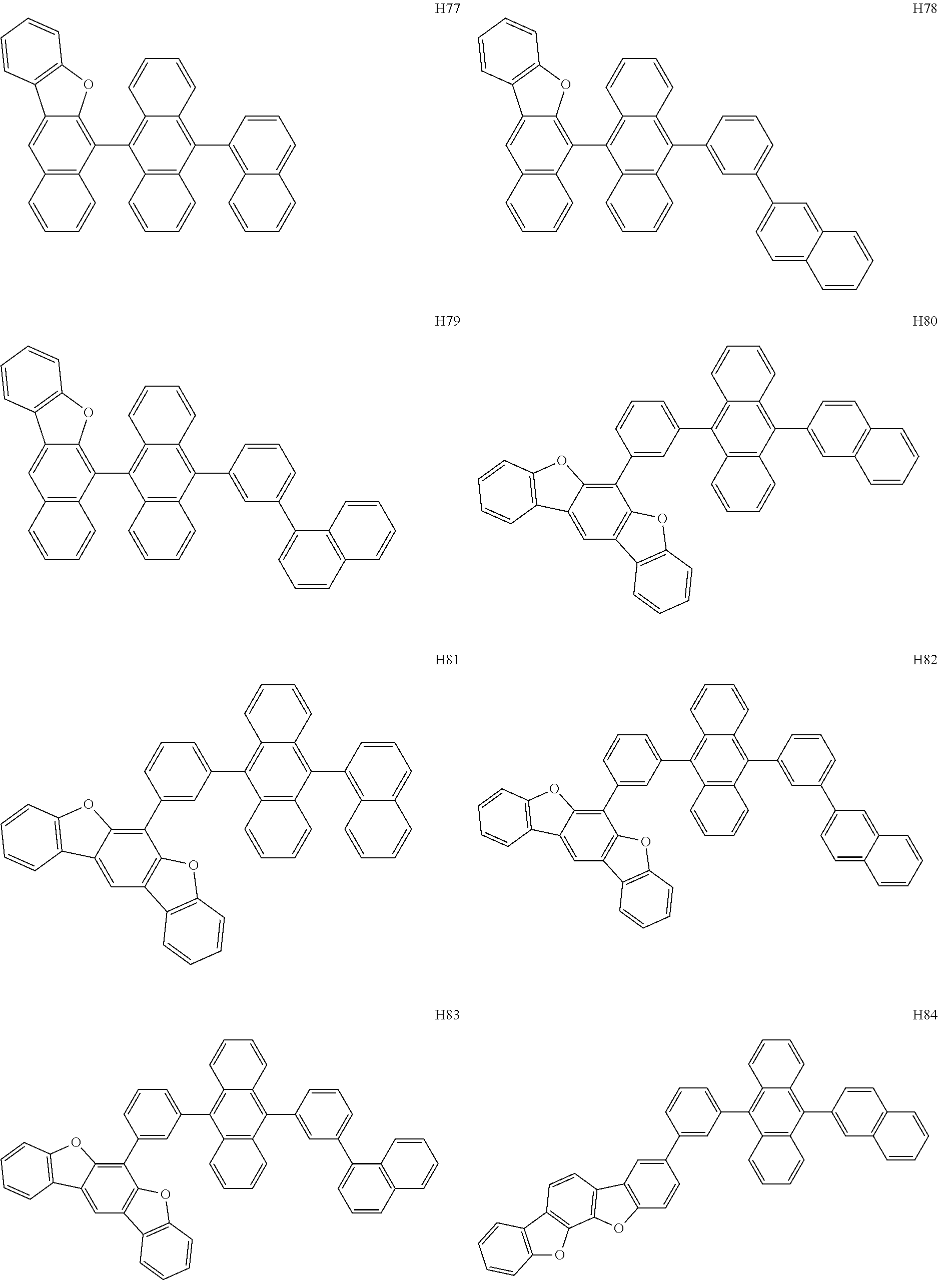

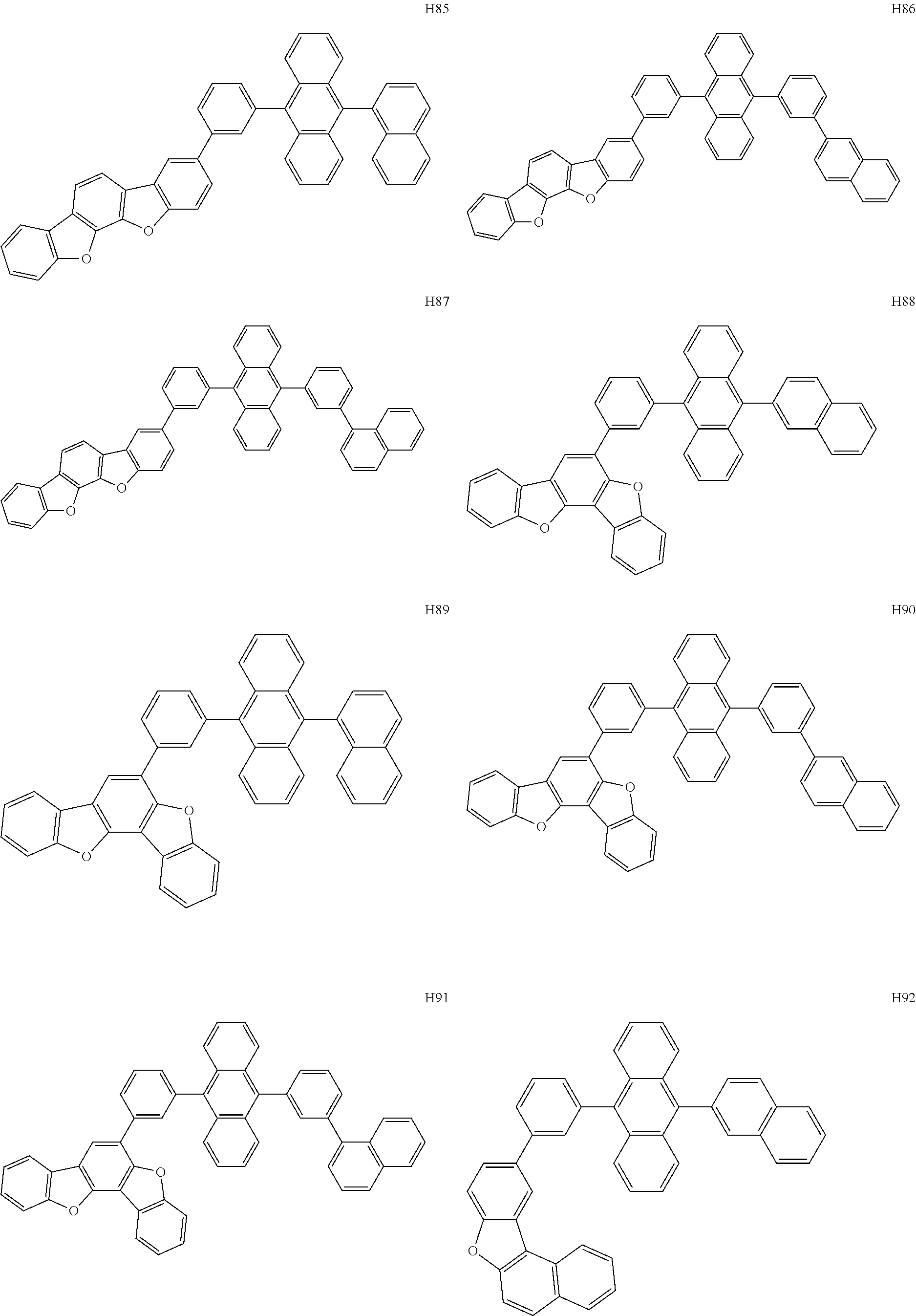

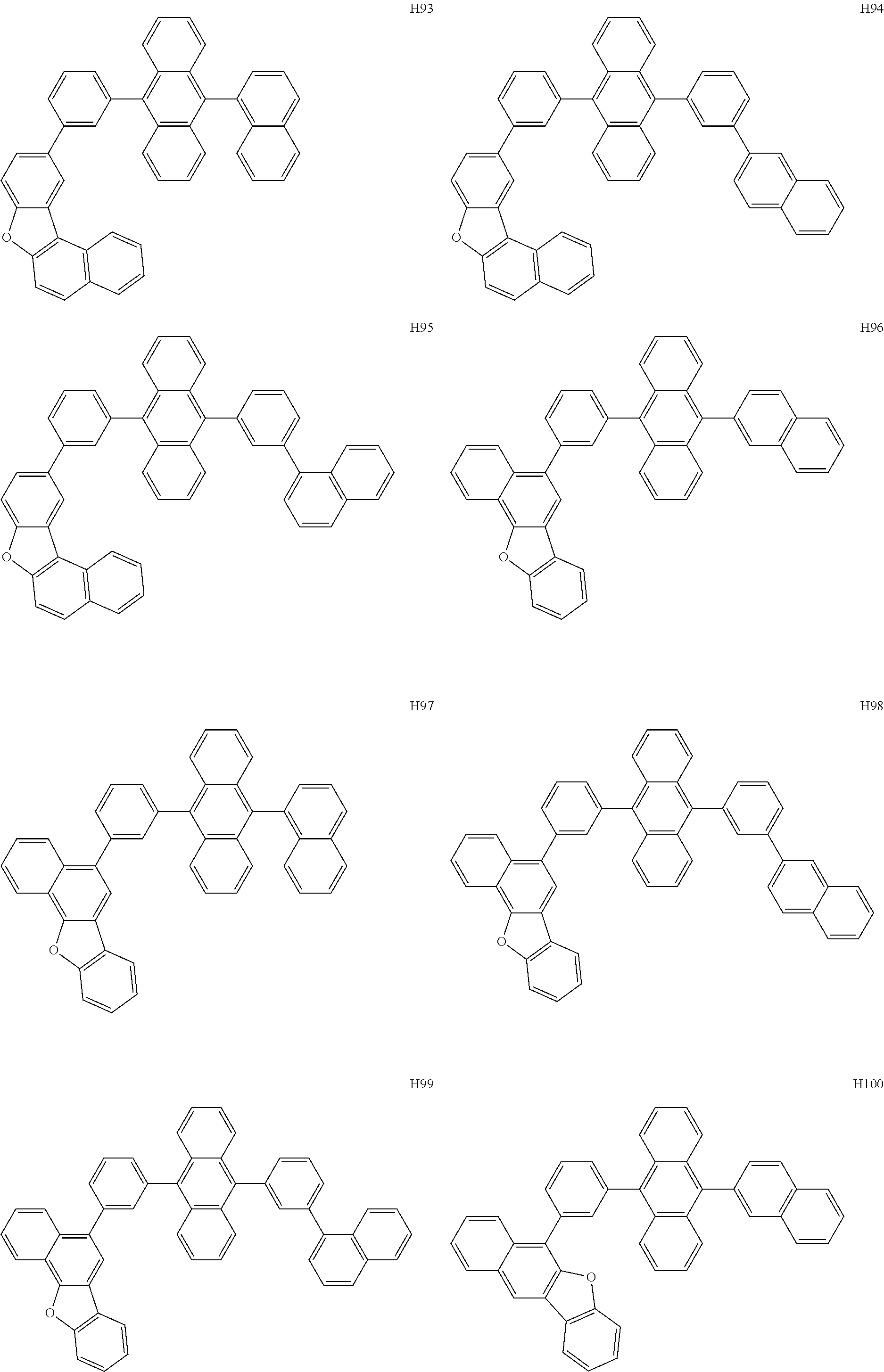

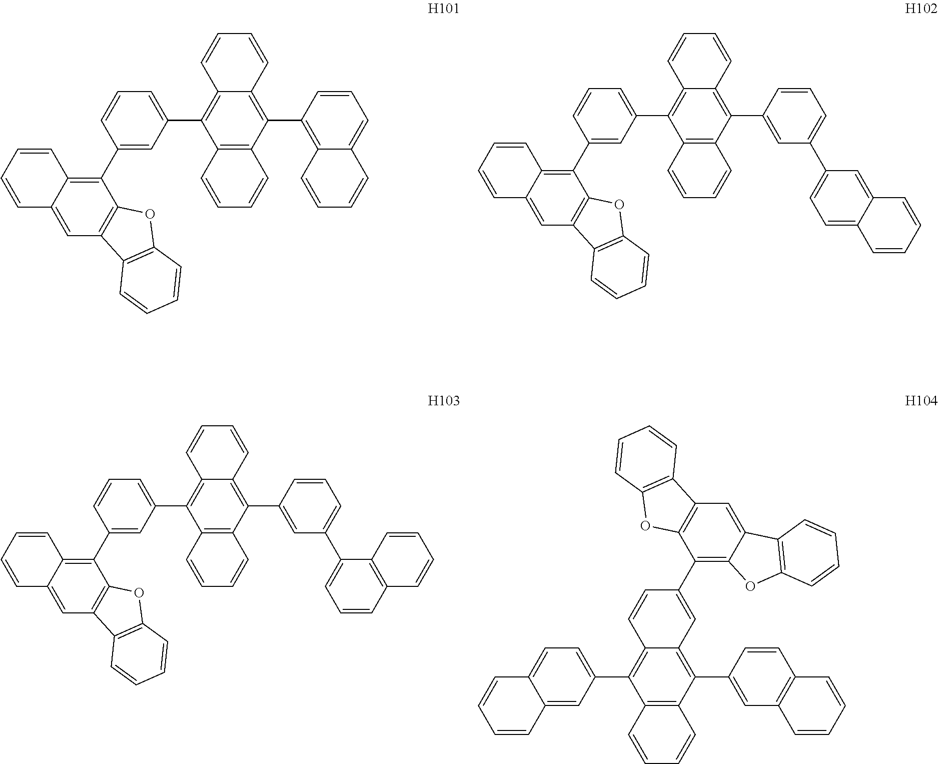 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

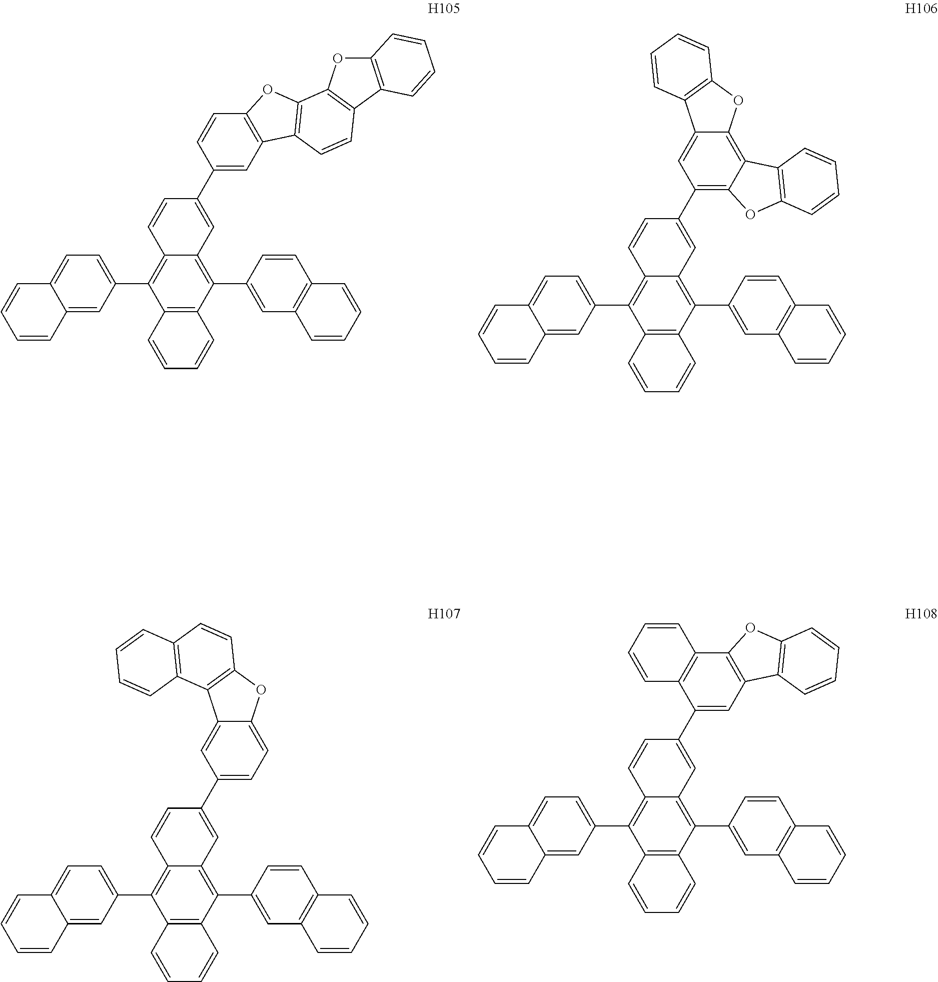 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

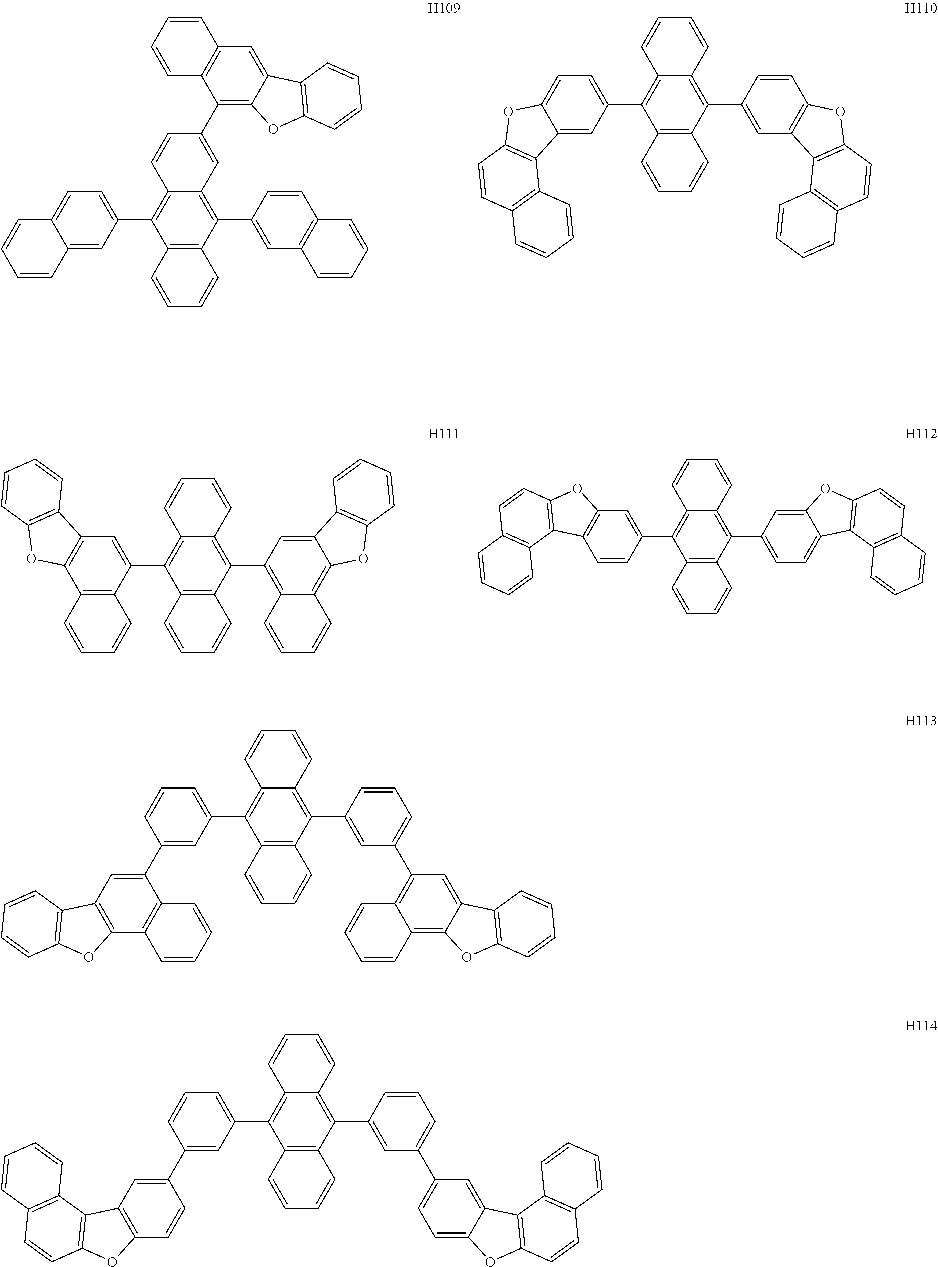

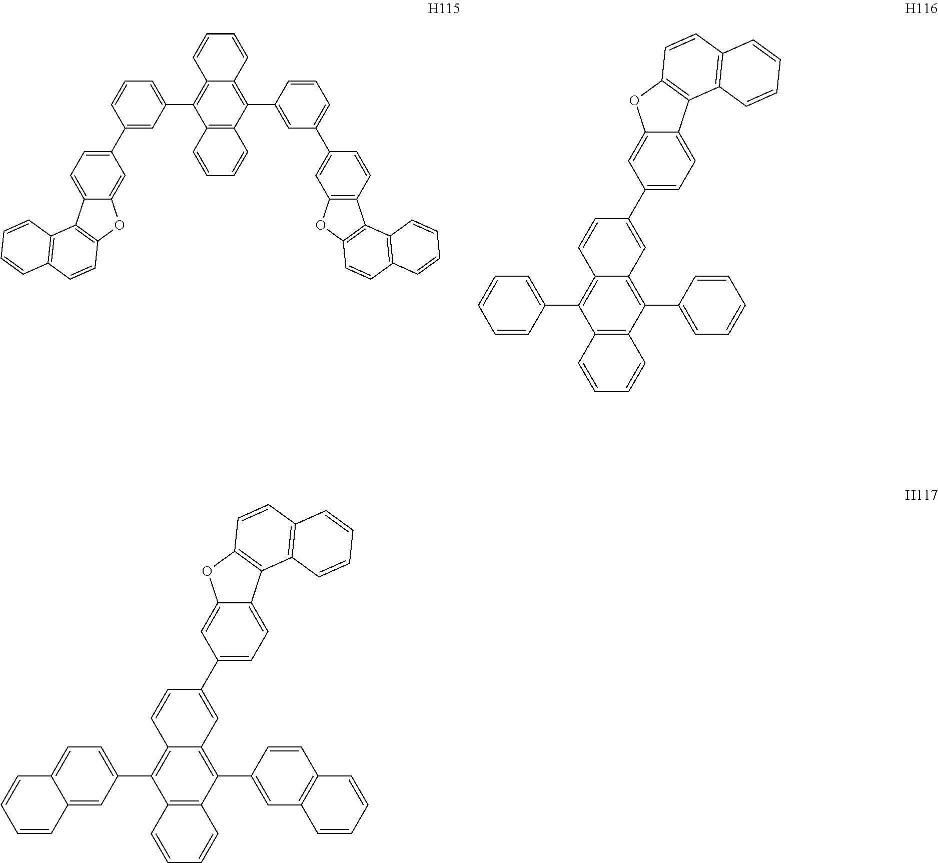 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1]

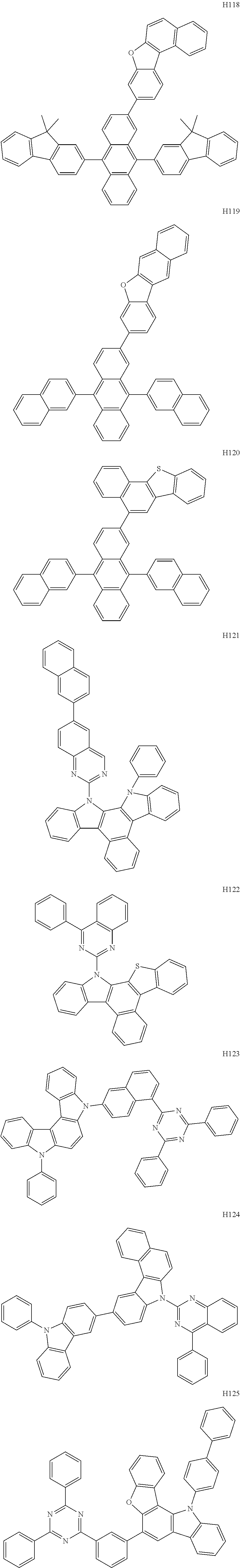

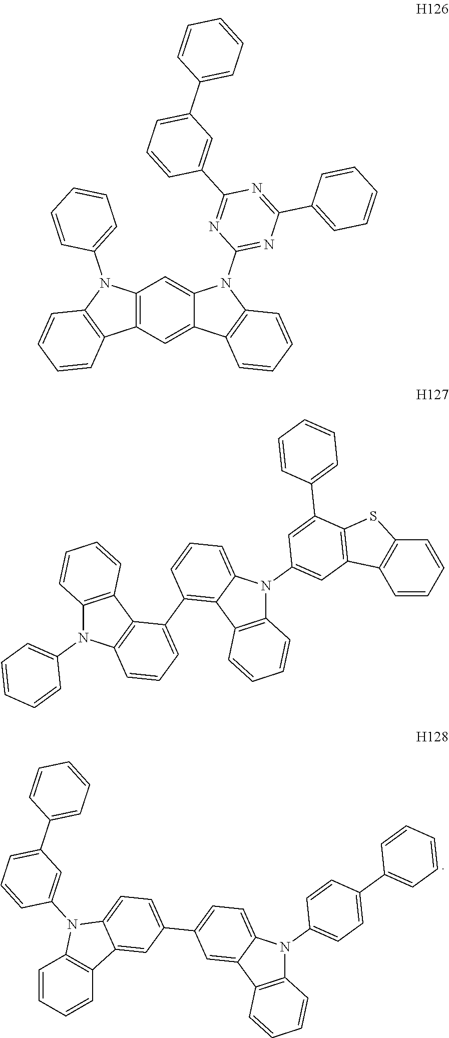 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

Phosphorescent dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, in one or more embodiments, the phosphorescent dopant may include an organometallic compound represented by Formula 401:

M(L₄₀₁)_{xc1}(L₄₀₂)_{xc2} Formula 401

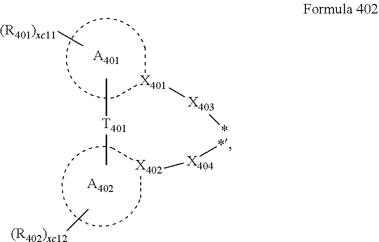

wherein, in Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is two or more, two or more of L₄₀₁(s) may be identical to or different from each other,

L₄₀₂ may be an organic ligand, and xc2 may be 0, 1, 2, 3, or 4, and when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *=C(Q₄₁₁)-*′,

X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each be the same as described herein with respect to Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each be the same as described herein with respect to Q₁,

xc11 and xc12 may each independently be an integer from 0 to 10, and

* and *′ in Formula 402 each indicates a binding site to M in Formula 401.

For example, in some embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁ (s) in two or more of L₄₀₁(s) may be optionally linked to each other via T₄₀₂, which is a linking group, and/or two ring A₄₀₂(s) may be optionally linked to each other via T₄₀₃, which is a linking group (see Compounds PD1 to PD4 and PD7). T₄₀₂ and T₄₀₃ may each be the same as described herein with respect to T₄₀₁.

L₄₀₂ in Formula 401 may be an organic ligand. For example, L₄₀₂ may include a halogen, a diketone group (for example, an acetylacetonate group), a carboxylic acid group (for example, a picolinate group), —C(═O), an isonitrile group, —CN group, a phosphorus-containing group (for example, a phosphine group, a phosphite group, etc.), or any combination thereof.

In one or more embodiments, the phosphorescent dopant may include, for example, one selected from among compounds PD1 to PD39, or any combination thereof:

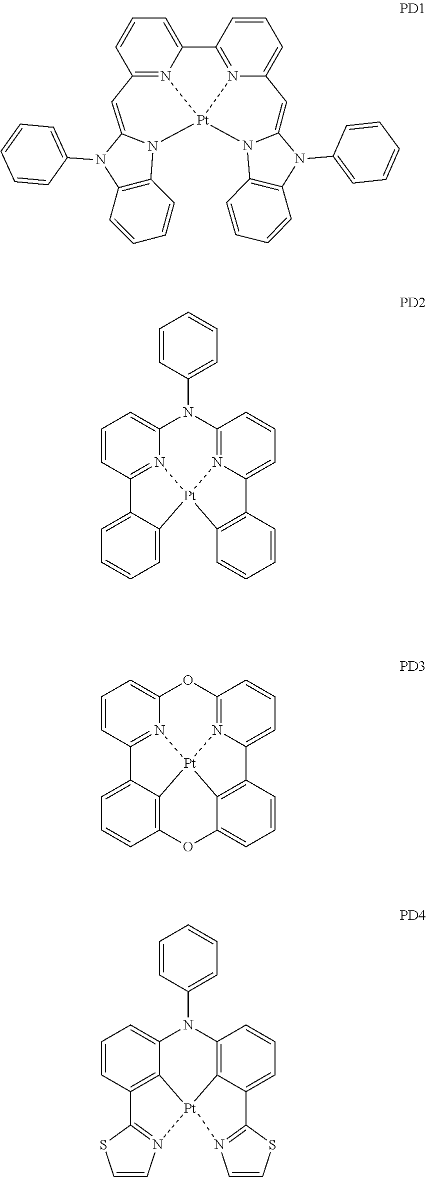

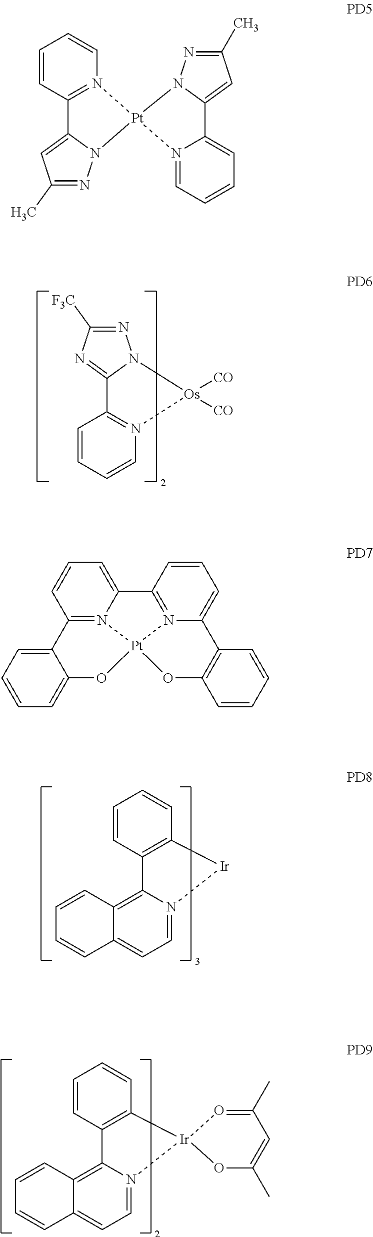

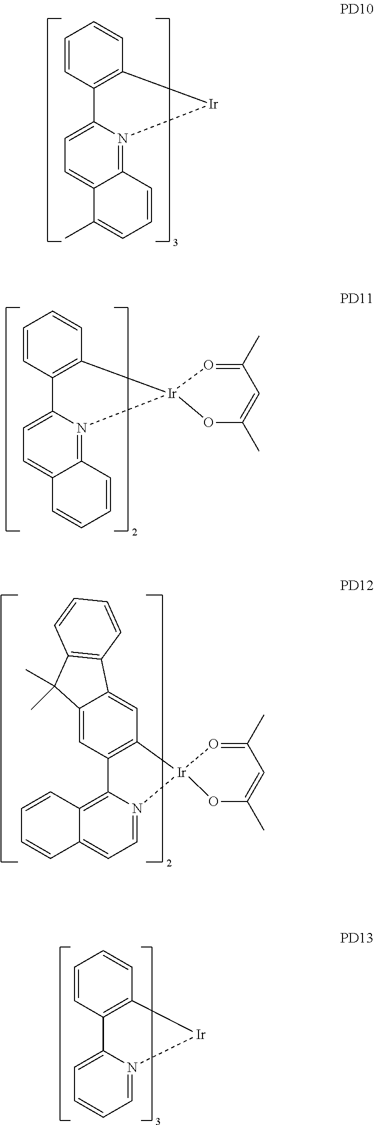

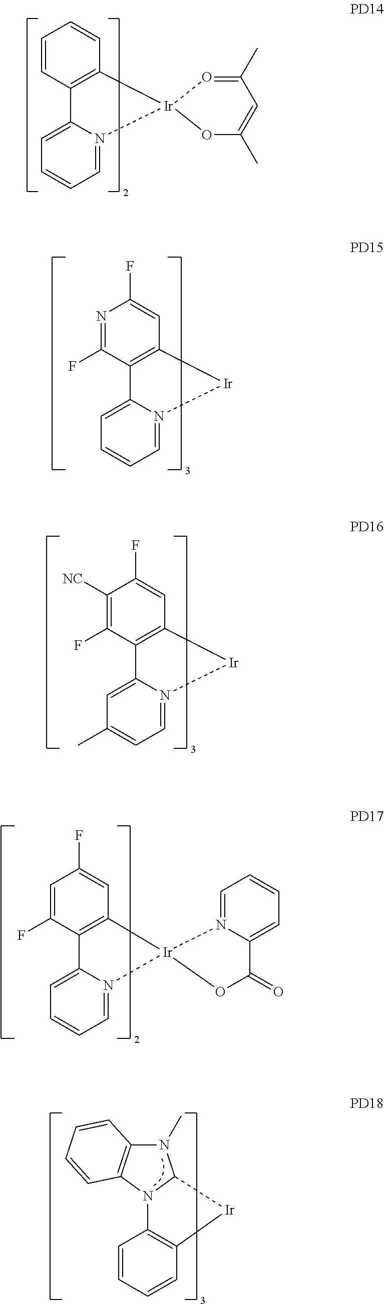

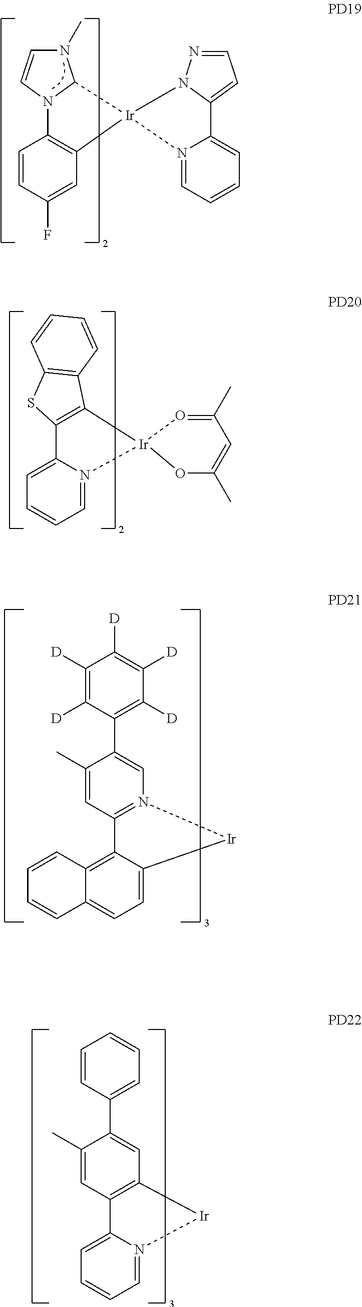

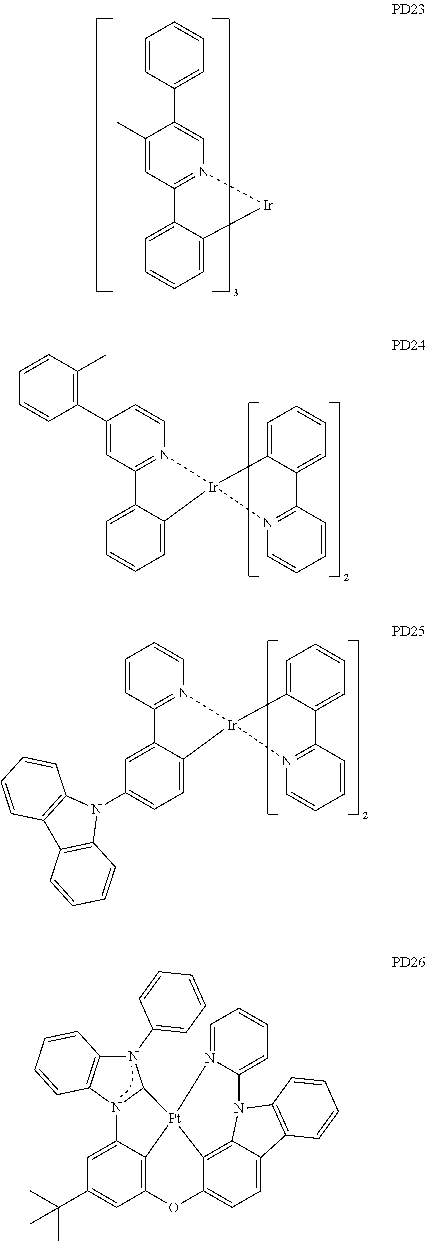

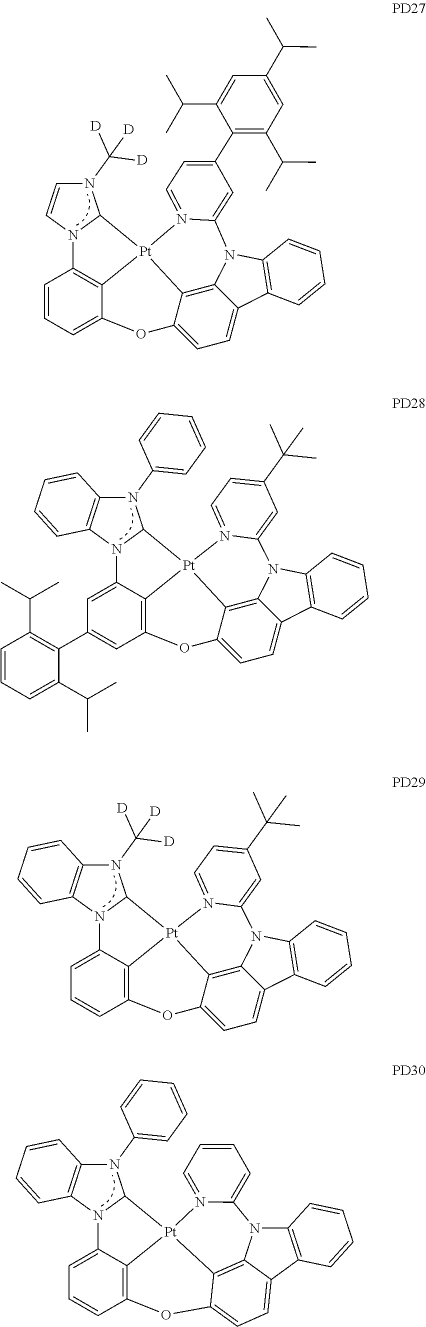

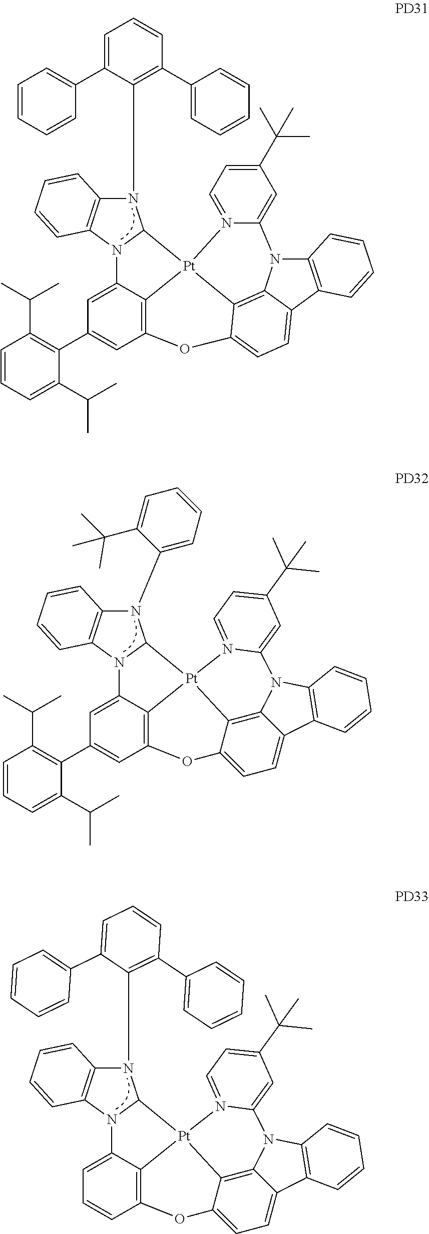

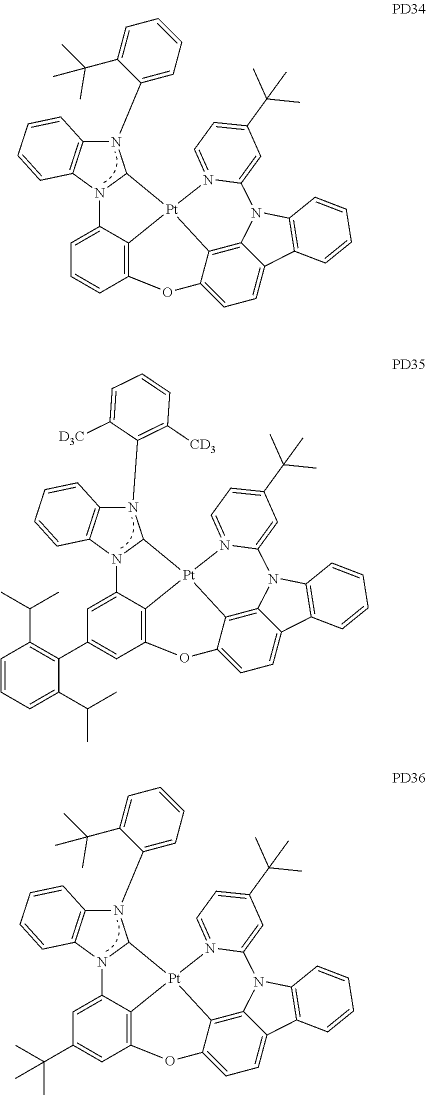

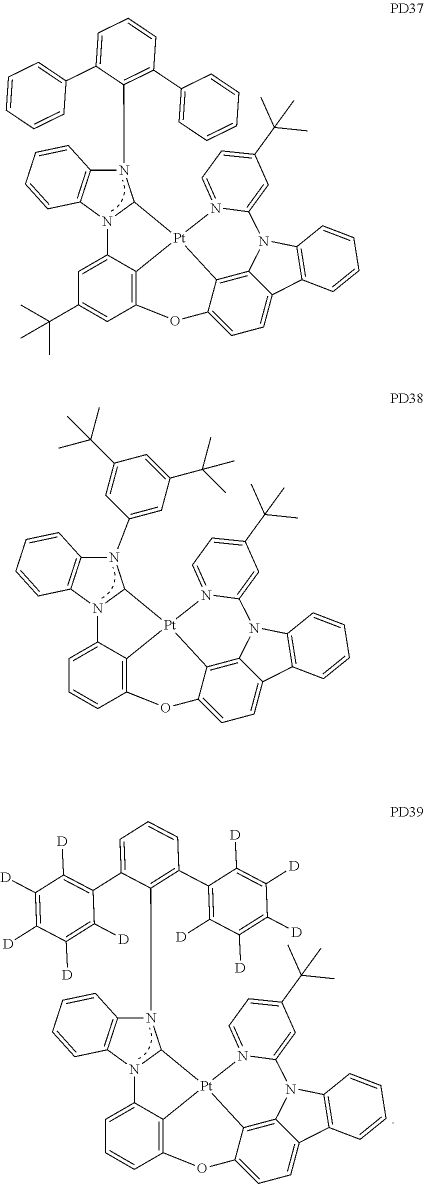

### Fluorescent Dopant

The fluorescent dopant may include an amine group-containing compound, for example, a compound represented by Formula 501, a styryl group-containing compound, or any combination thereof:

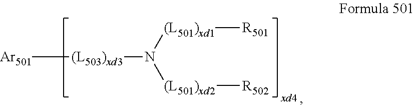

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1,2, 3,4, 5, or 6.

For example, in some embodiments, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

In one or more embodiments, the fluorescent dopant may include: at least one selected from among Compounds FD1 to FD37; 4,4′-bis(2,2-diphenylvinyl)-1,1′-biphenyl (DPVBi); 4,4′-bis[4-(N,N-diphenylamino)styryl]biphenyl (DPAVBi); or any combination thereof:

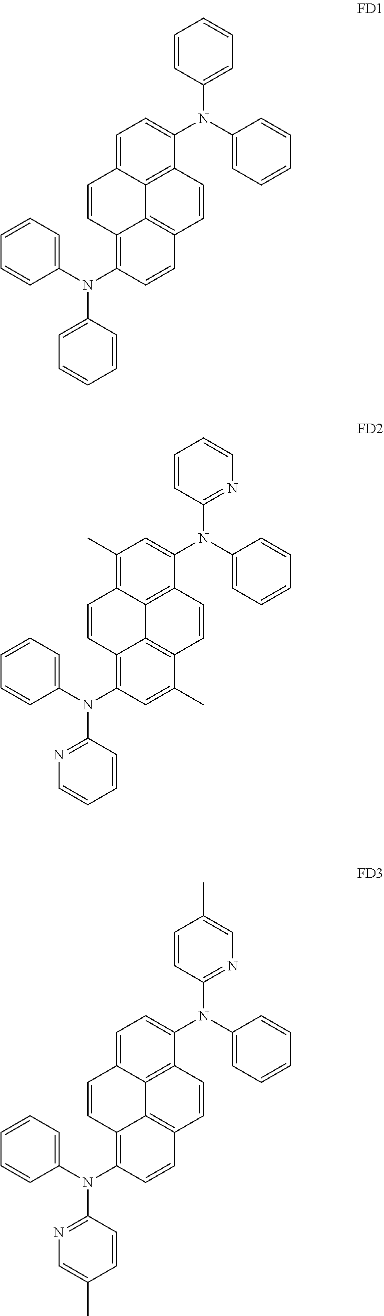 [SMILES: c1ccc(N(c2ccccc2)c2ccc3ccc4c(N(c5ccccc5)c5ccccc5)ccc5ccc2c3c54)cc1 ; Cc1cc(N(c2ccccc2)c2ccccn2)c2ccc3c(C)cc(N(c4ccccc4)c4ccccn4)c4ccc1c2c34 ; Cc1ccc(N(c2ccccc2)c2ccc3ccc4c(N(c5ccccc5)c5ccc(C)cn5)ccc5ccc2c3c54)nc1]

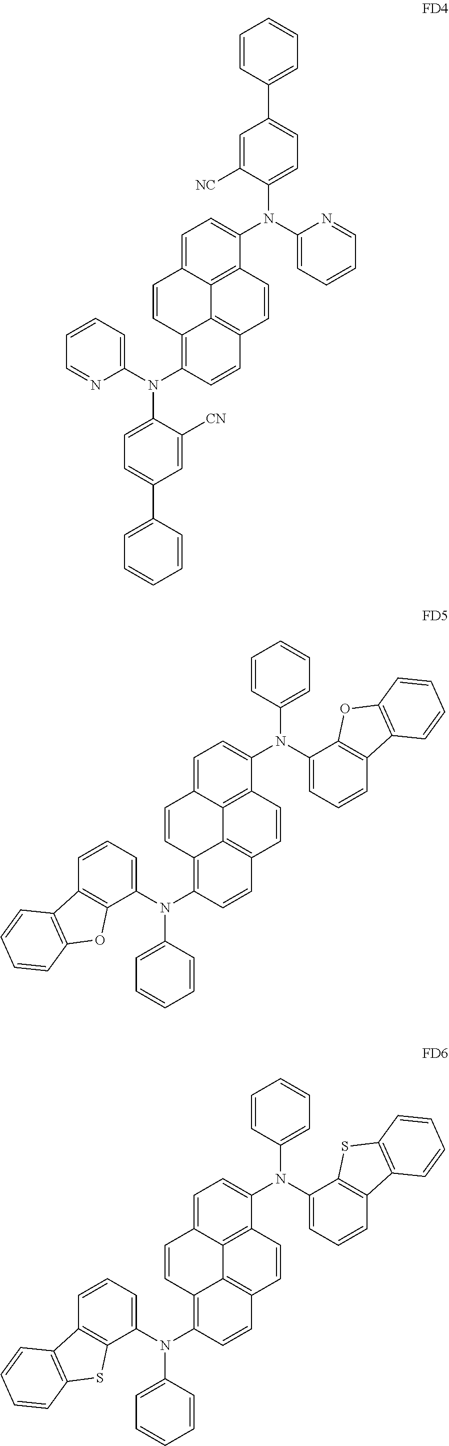 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

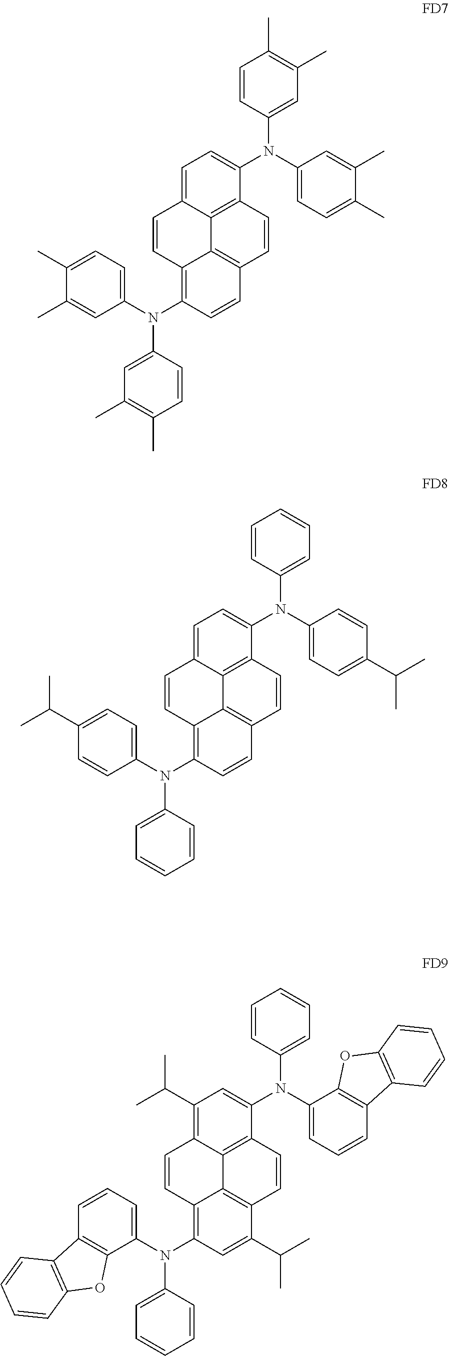

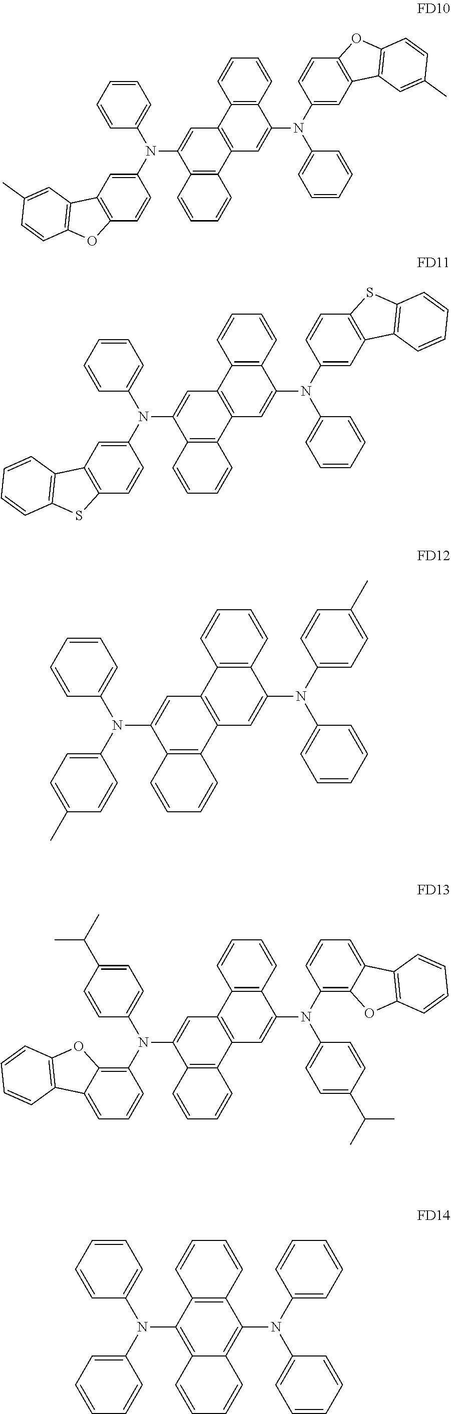

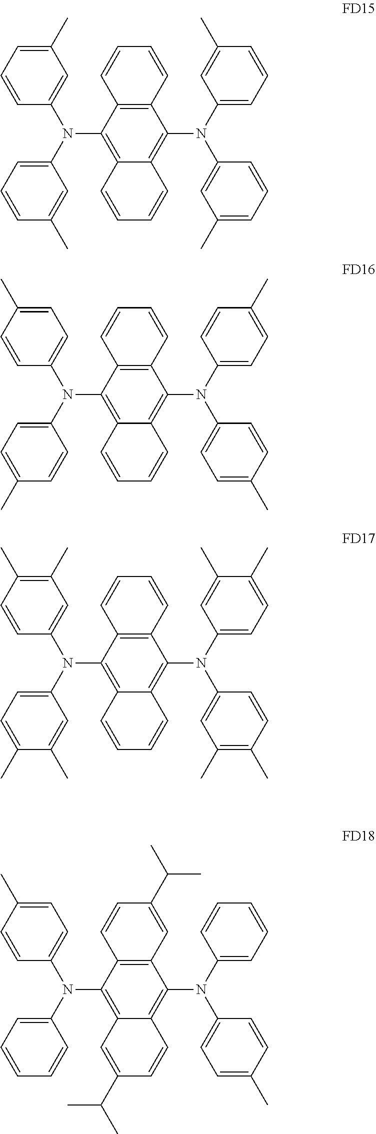

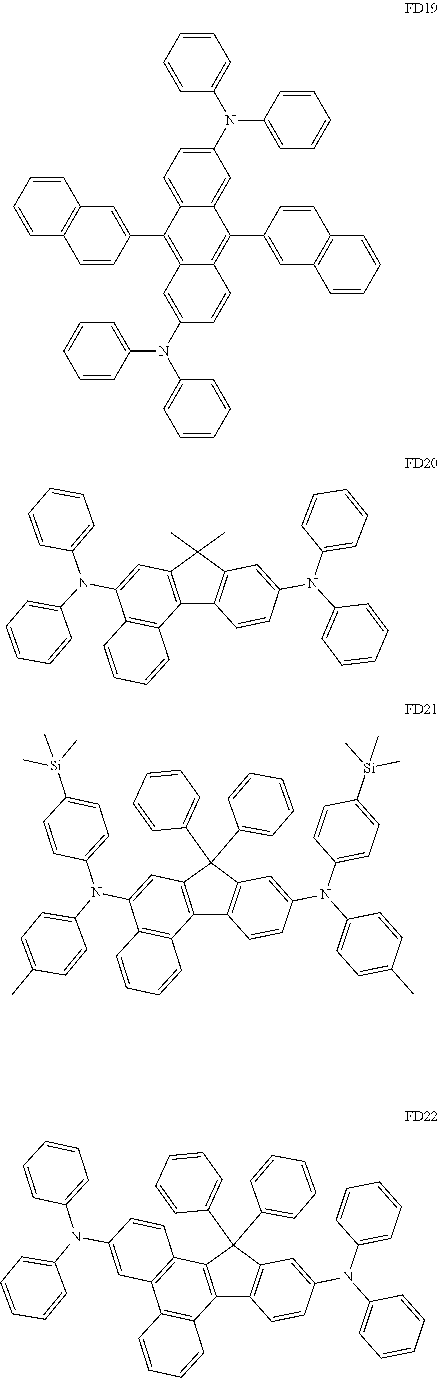

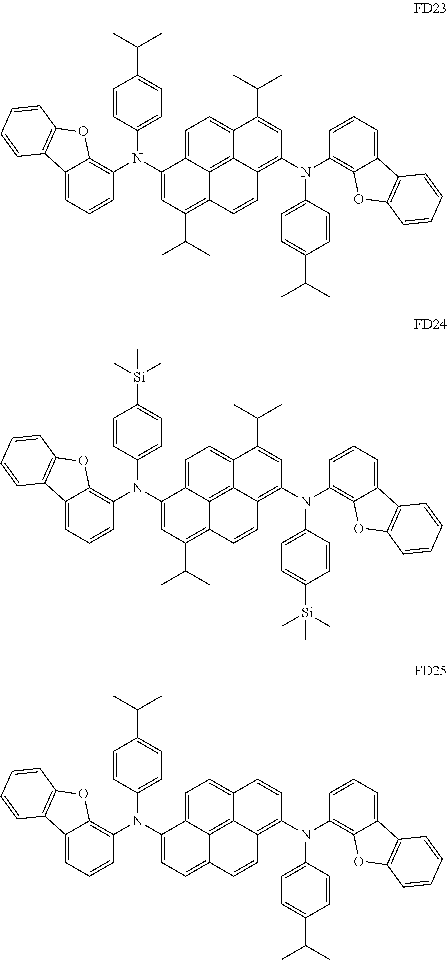 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1]

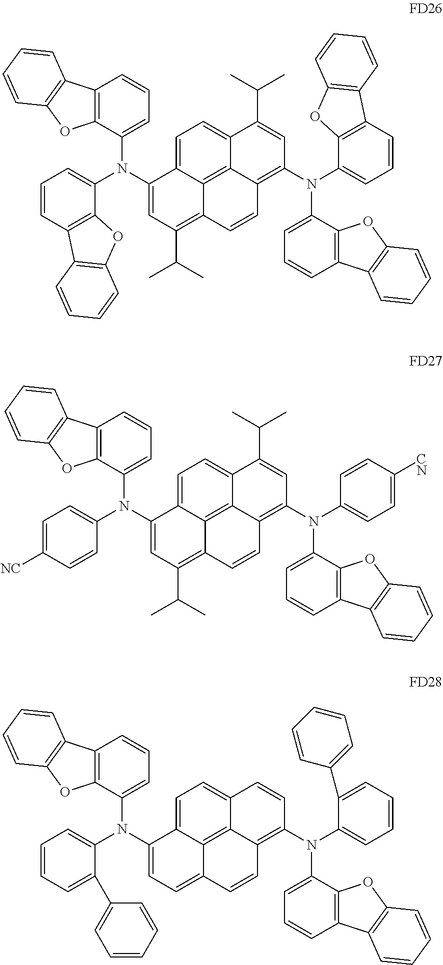 [SMILES: CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34 ; CC(C)c1cc(N(c2ccc(C#N)cc2)c2cccc3c2oc2ccccc23)c2ccc3c(C(C)C)cc(N(c4ccc(C#N)cc4)c4cccc5c4oc4ccccc45)c4ccc1c2c34 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1]

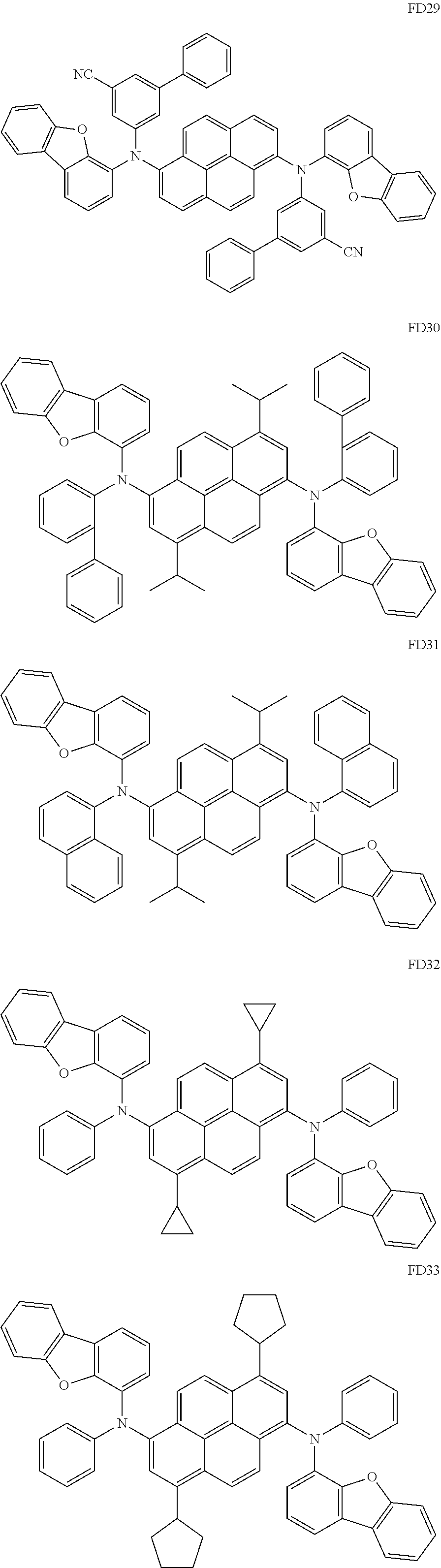

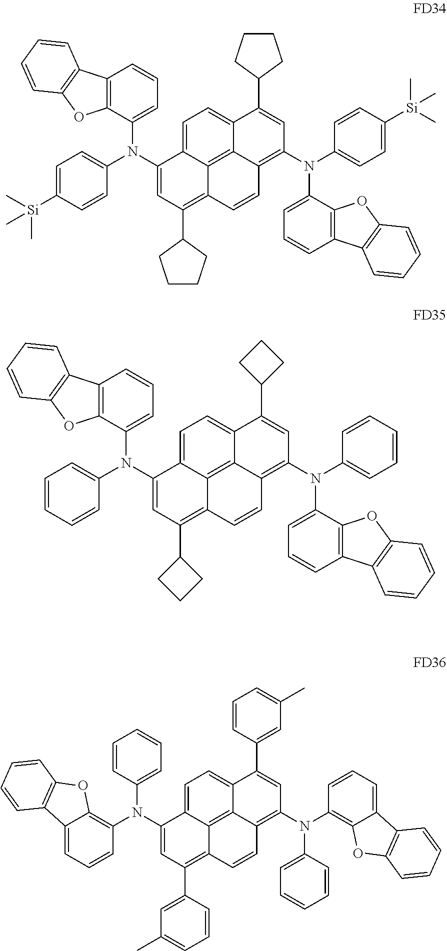

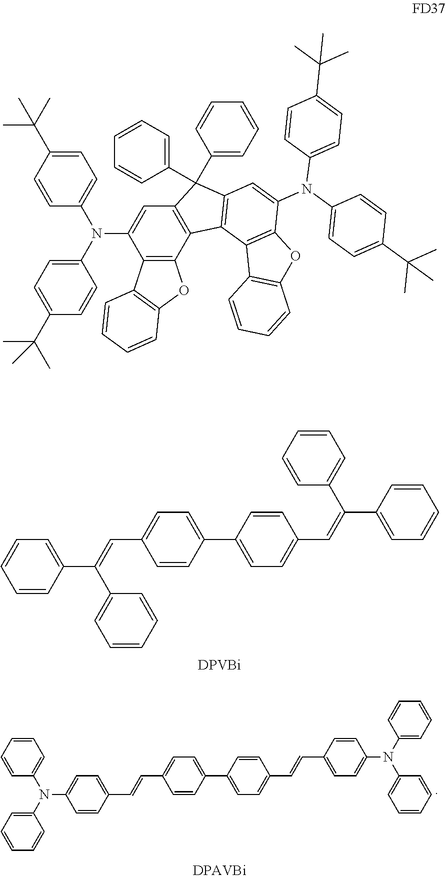

### Delayed Fluorescence Material

In one or more embodiments, the emission layer may include a delayed fluorescence material.

In the present disclosure, the delayed fluorescence material may be selected from compounds capable of emitting delayed fluorescent light based on a delayed fluorescence emission mechanism.

The delayed fluorescence material included in the emission layer may act as a host or a dopant depending on the type or kind of other materials included in the emission layer.

In one or more embodiments, a difference between a triplet energy level of the delayed fluorescence material and a singlet energy level of the delayed fluorescence material may be greater than or equal to 0 eV and less than or equal to 0.5 eV. When the difference between the triplet energy level of the delayed fluorescence material and the singlet energy level of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, in one or more embodiments, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and/or ii) a material including a C₈-C₆₀ polycyclic group in which two or more cyclic groups are condensed while sharing boron (B).

Non-limiting examples of the delayed fluorescence material may include at least one selected from among Compounds DF1 to DF14:

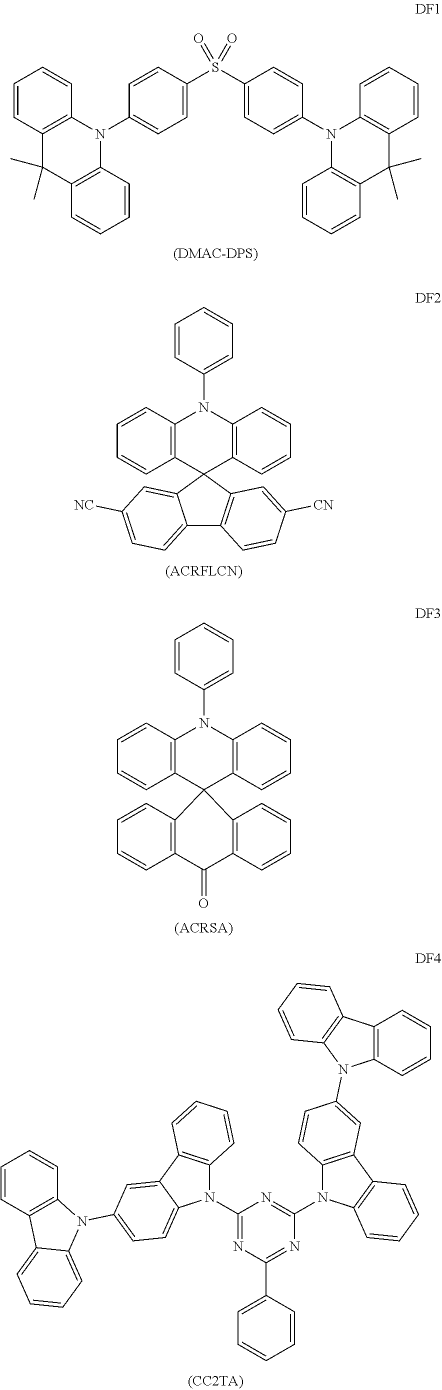

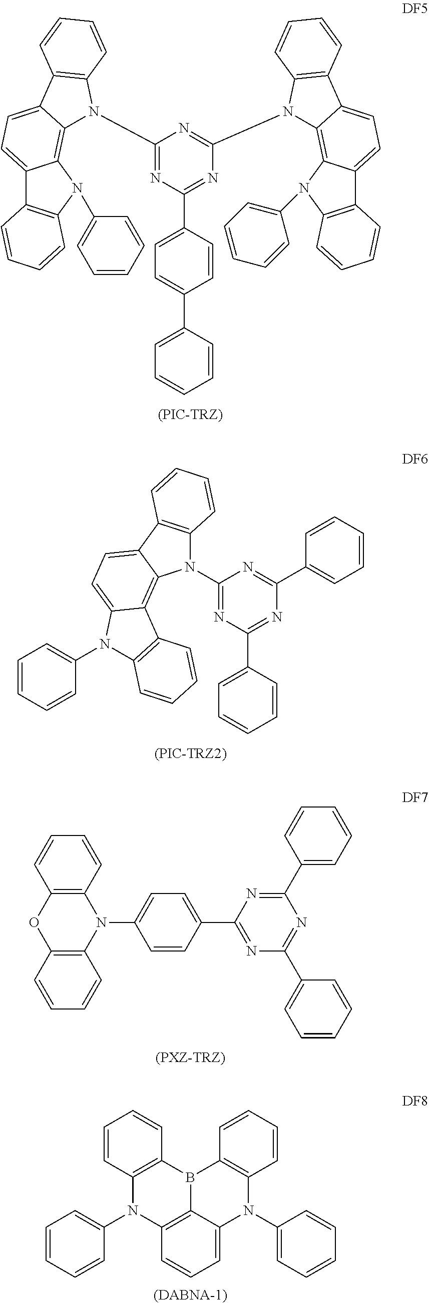

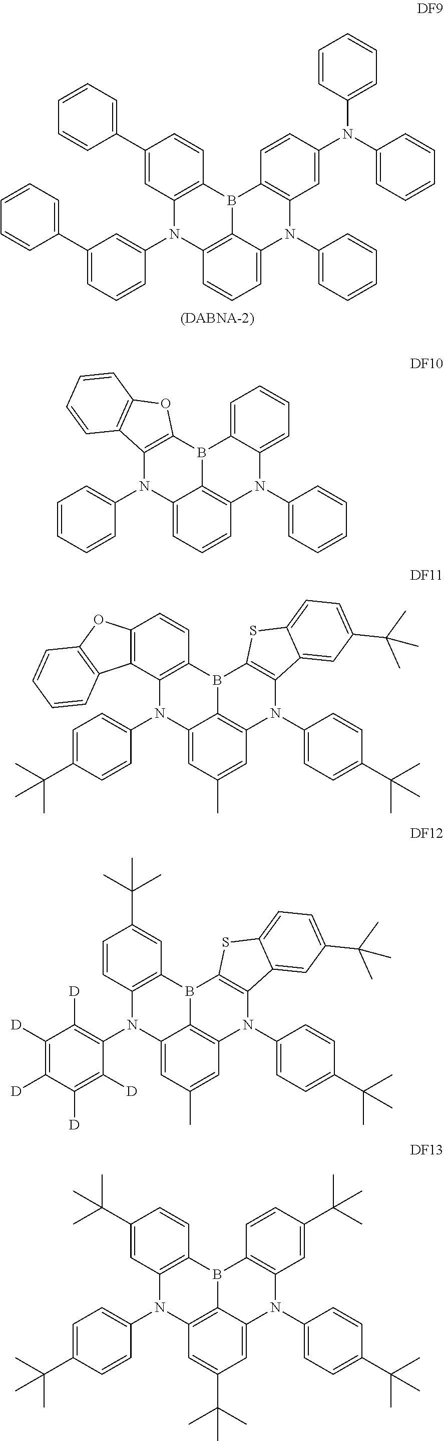

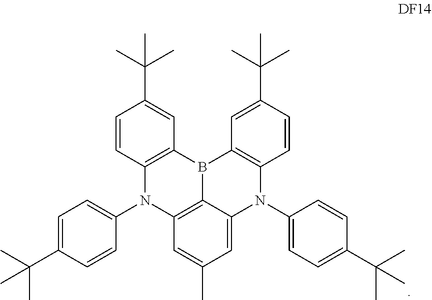 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

In one or more embodiments, the emission layer may include a quantum dot.

The term “quantum dot” as utilized herein refers to a crystal of a semiconductor compound, and may include any material capable of emitting light of one or more suitable emission wavelengths according to the size of the crystal.

A diameter of the quantum dot may be, for example, in a range of about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a wet chemical process, a metal organic chemical vapor deposition process, a molecular beam epitaxy process, or any process similar thereto.

The wet chemical process is a method including mixing a precursor material of a quantum dot with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles may be controlled or selected through a process which costs lower, and is easier than vapor deposition methods, such as metal organic chemical vapor deposition (MOCVD) or molecular beam epitaxy (MBE),

The quantum dot may include Group II-VI semiconductor compounds, Group III-V semiconductor compounds, Group III-VI semiconductor compounds, Group 1-III-VI semiconductor compounds, Group IV-VI semiconductor compounds, Group IV elements or compounds, or any combination thereof.

Non-limiting examples of the Group II-VI semiconductor compound may include (e.g., be) a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or one or more combinations thereof.

Non-limiting examples of the Group III-V semiconductor compound may include (e.g., be): a binary compound, such as GaN, GaP, GaAs, GaSb, AlN, AlP, AlAs, AISb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or one or more combinations thereof. In some embodiments, the Group III-V semiconductor compound may further include a Group II element. Non-limiting examples of the Group III-V semiconductor compound further including a Group II element may be InZnP, InGaZnP, InAlZnP, etc.

Non-limiting examples of the Group III-VI semiconductor compound may be: a binary compound, such as GaS, GaSe, Ga₂Se₃, GaTe, InS, InSe, In₂S₃, In₂Se₃, and/or InTe; a ternary compound, such as InGaS₃, and/or InGaSe₃; or one or more combinations thereof.

Non-limiting examples of the Group I-III-VI semiconductor compound may be: a ternary compound, such as AgInS, AgInS₂, CuInS, CuInS₂, CuGaO₂, AgGaO₂, and/or AgAlO₂; or one or more combinations thereof.

Non-limiting examples of the Group IV-VI semiconductor compound may be: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or one or more combinations thereof.

The Group IV element or compound may include: a single element compound, such as Si or Ge; a binary compound, such as SiC or SiGe; or any combination thereof.

Each element included in a multi-element compound such as the binary compound, the ternary compound, and the quaternary compound may be present at a substantially uniform concentration or non-substantially uniform concentration in a particle.

In some embodiments, the quantum dot may have a single structure in which the concentration of each element in the quantum dot is substantially uniform, or a core-shell dual structure. For example, a material included in the core and a material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be an oxide of metal, metalloid, or non-metal, a semiconductor compound, and any combination thereof.

Non-limiting examples of the oxide of metal, metalloid, or non-metal may be a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; and any combination thereof. Examples of the semiconductor compound may be, as described herein, a Group II-VI semiconductor compound; a Group III-V semiconductor compound; a Group III-VI semiconductor compound; a Group I-III-VI semiconductor compound; a Group IV-VI semiconductor compound; or one or more combinations thereof. For example, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission spectrum of the quantum dot may be about 45 nm or less, for example, about 40 nm or less, for example, about 30 nm or less, and within these ranges, color purity or color reproducibility of the quantum dot may be increased. In some embodiments, because the light emitted through the quantum dot is emitted in all directions, the wide viewing angle may be improved.

In one or more embodiments, the quantum dot may be in the form of a spherical particle, a pyramidal particle, a multi-arm particle, a cubic nanoparticle, a nanotube particle, a nanowire particle, a nanofiber particle, or a nanoplate particle.

Because the energy band gap of the quantum dot may be adjusted by controlling the size of the quantum dot, light having one or more suitable wavelength bands may be obtained from a quantum dot emission layer. Accordingly, by utilizing quantum dots of different sizes, a light-emitting device that emits light of one or more suitable wavelengths may be implemented. In one or more embodiments, the size of the quantum dots may be selected to enable the quantum dots to emit red, green and/or blue light. In some embodiments, the quantum dots with suitable sizes may be configured to emit white light by combination of light of one or more suitable colors.

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron-transporting region may include a buffer layer, a hole-blocking layer, an electron control layer, an electron-transporting layer, an electron injection layer, or any combination thereof.

For example, in one or more embodiments, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the constituting layers of each structure being sequentially stacked from the emission layer in the stated order.

In one or more embodiments, the electron transport region (for example, the buffer layer, the hole-blocking layer, the electron control layer, or the electron transport layer in the electron transport region) may include a metal-free compound including at least one π electron-deficient nitrogen-containing C₁-C₆₀ cyclic group.

For example, in some embodiments, the electron transport region may include a compound represented by Formula 601:

[Ar₆₀₁]ₓₑ₁₁-[(L₆₀₁)ₓₑ₁-R₆₀₁]ₓₑ₂₁, Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each be the same as described herein with respect to Q₁,

xe21 may be 1, 2, 3, 4, or 5, and

at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In some embodiments, Ar₆₀₁ in Formula 601 may be a substituted or unsubstituted anthracene group.

In some embodiments, the electron transport region may include a compound represented by Formula 601-1:

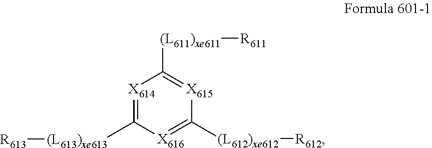

wherein, in Formula 601-1,

X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,

L₆₁₁ to L₆₁₃ may each be the same as described herein with respect to L₆₀₁,

xe611 to xe613 may each be the same as described herein with respect to xe1,

R₆₁₁ to R₆₁₃ may each be the same as described herein with respect to R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in some embodiments, xe1 and xe611 to xe613 in Formulae 601 and 601-1 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), tris(8-hydroxyquinolinato)aluminum (Alq₃), bis(2-methyl-8-quinolinolato-N1,08)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), or any combination thereof:

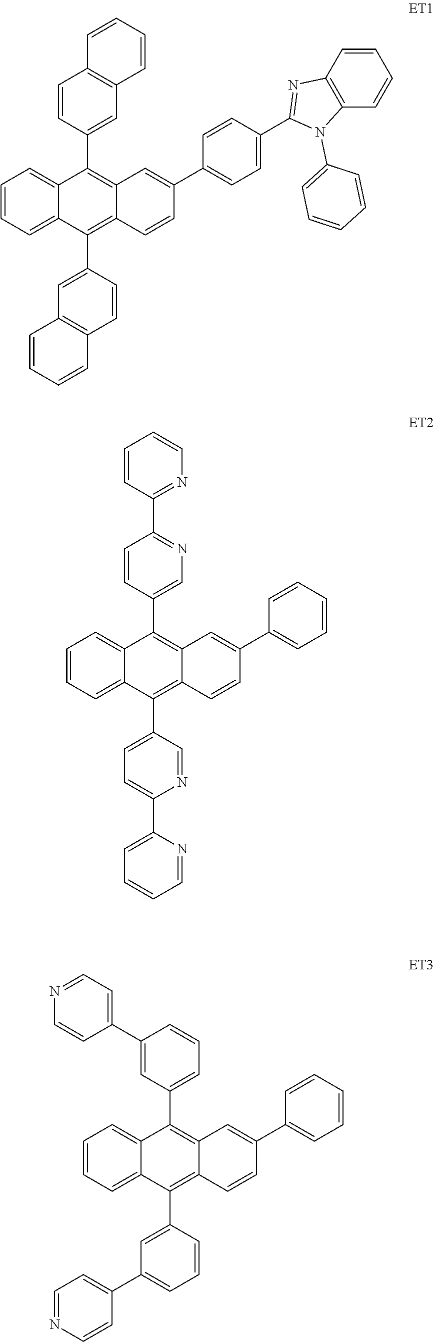

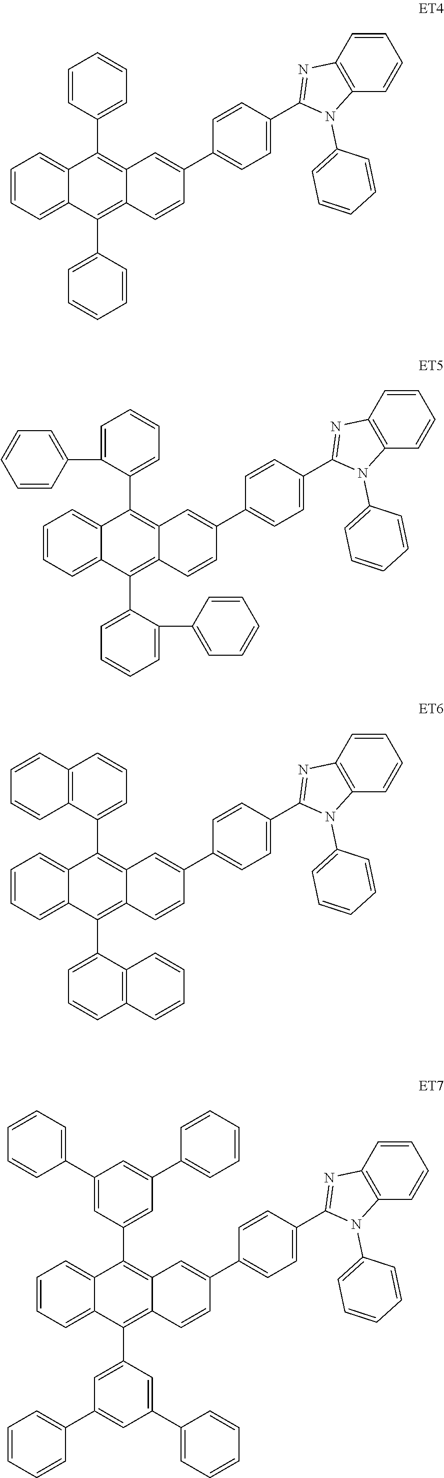 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

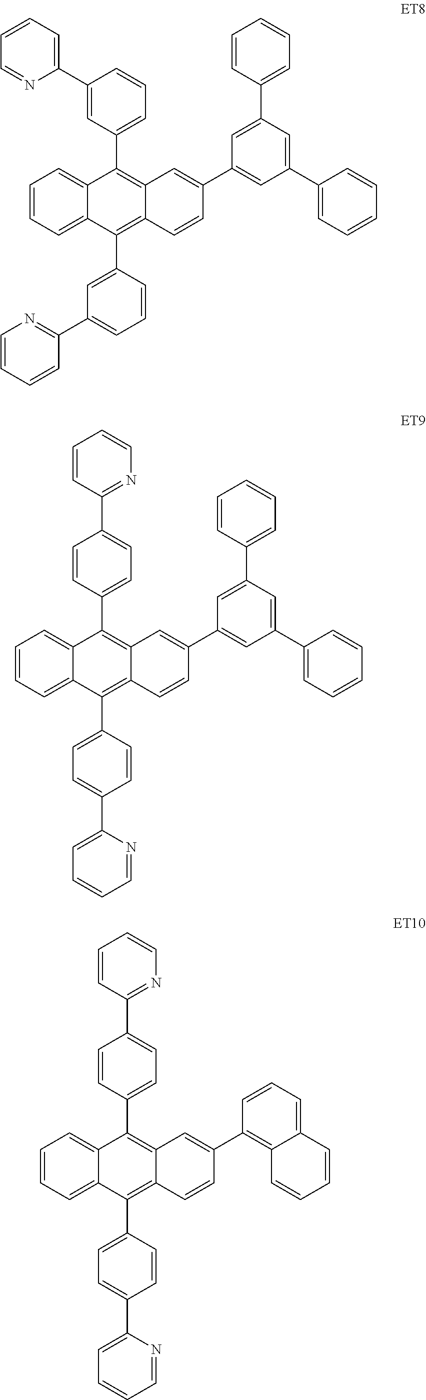

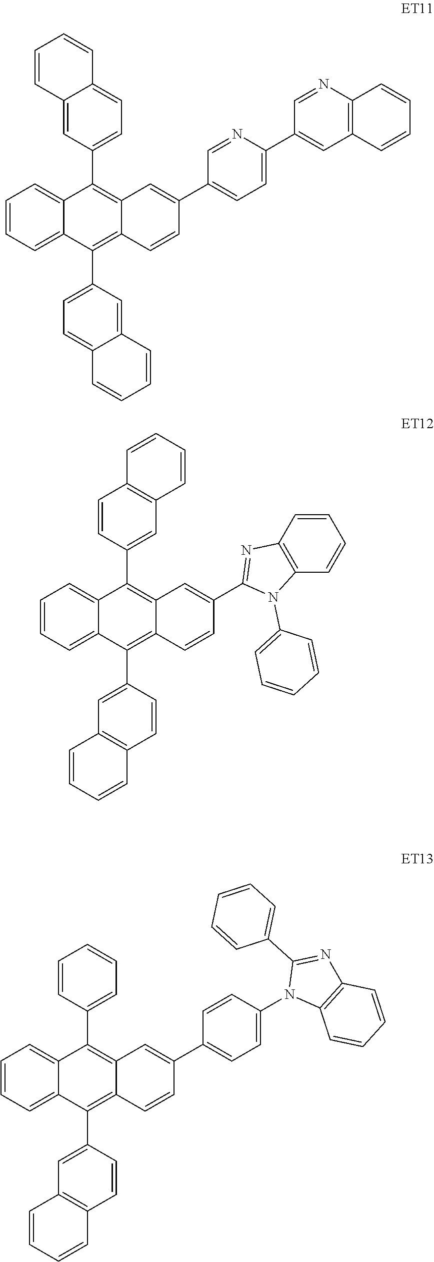 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

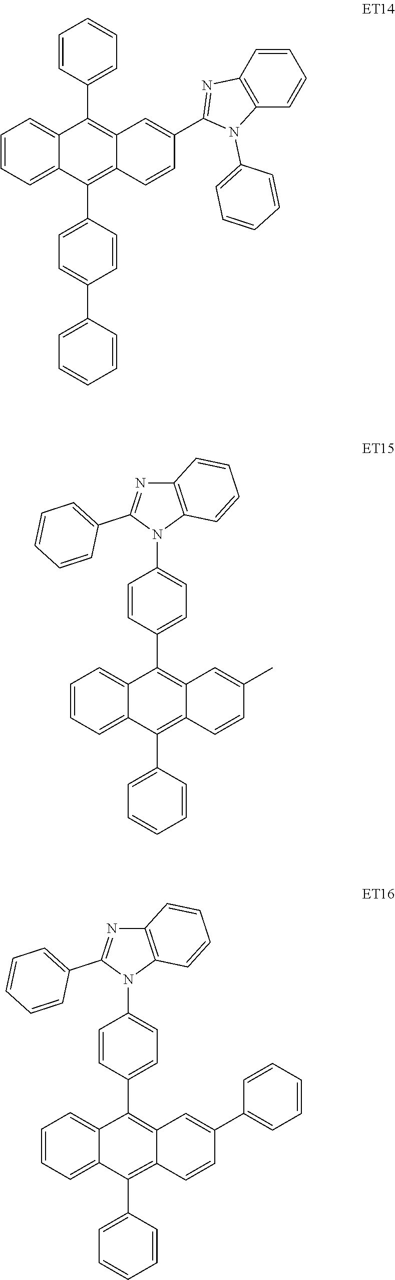 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

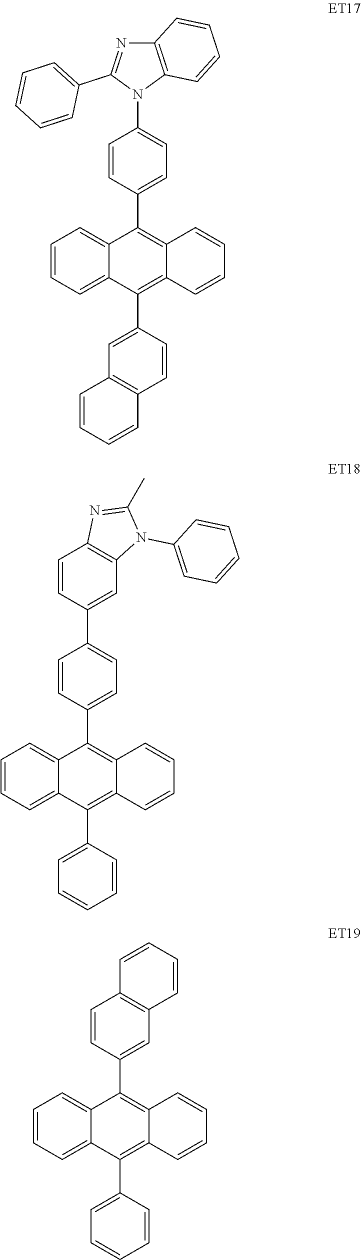

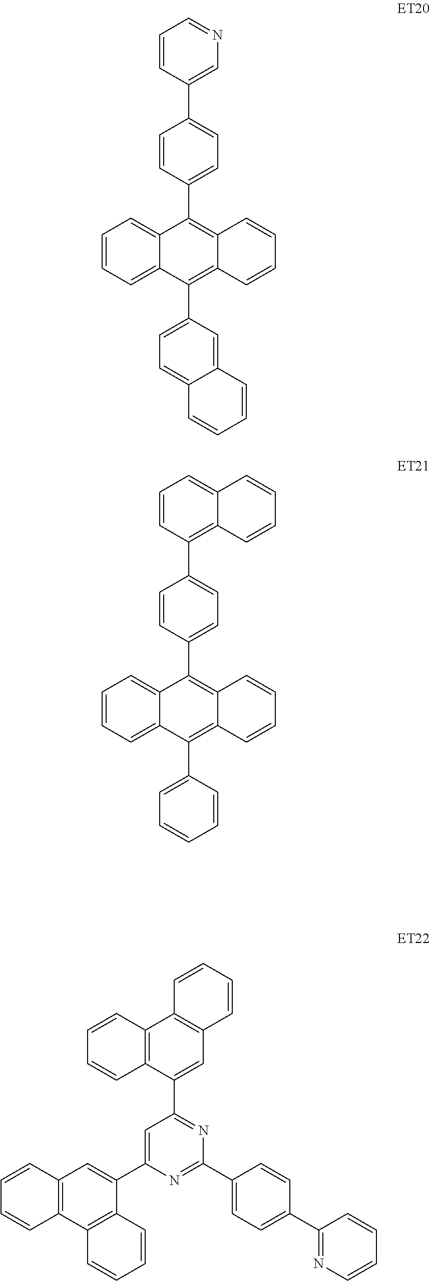

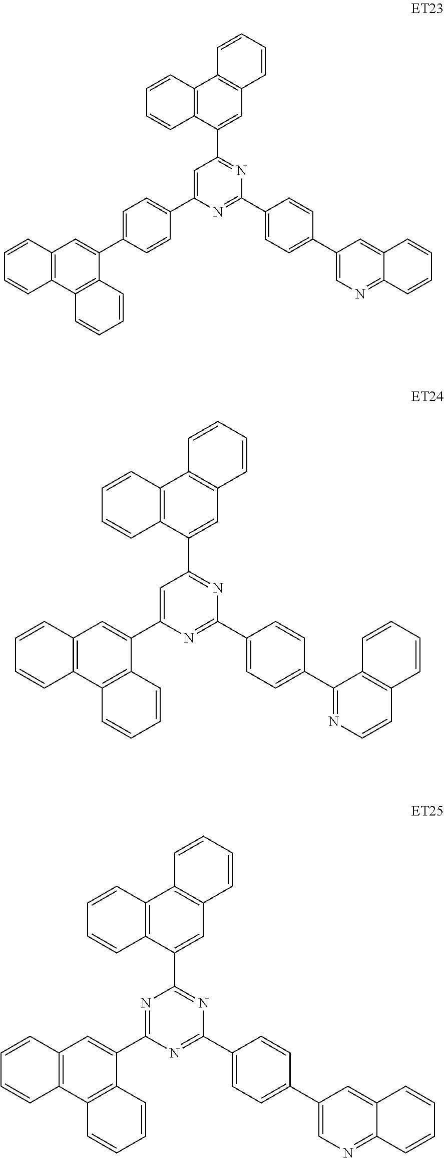 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

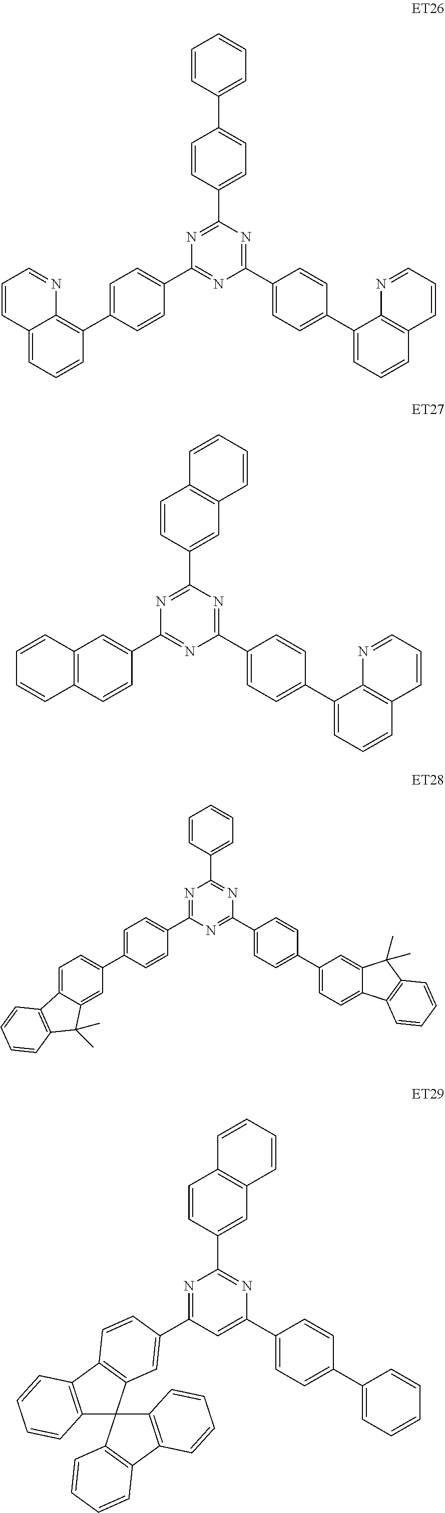 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

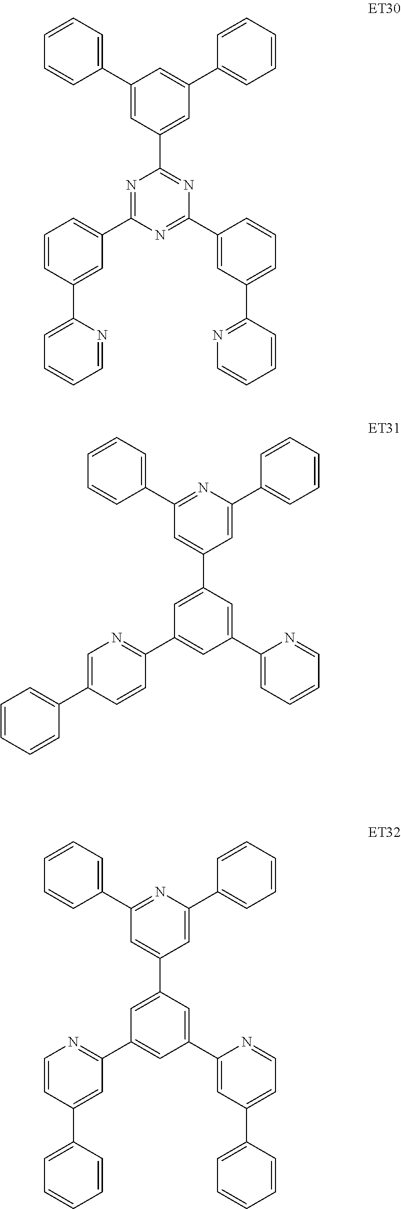 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

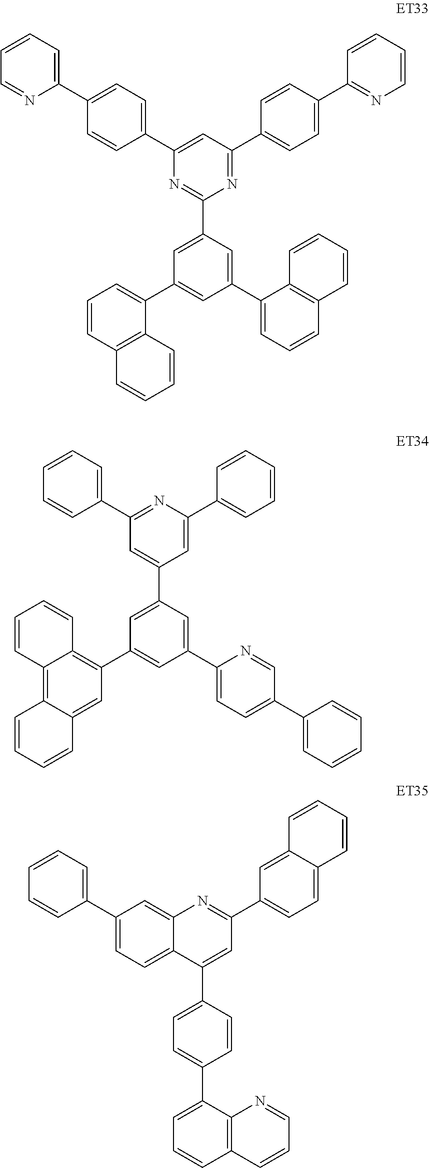

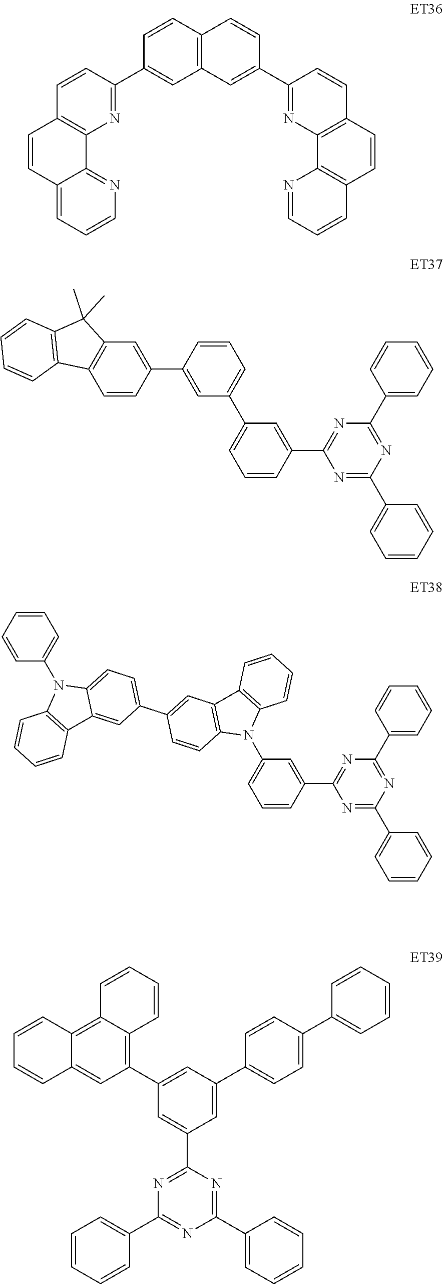 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

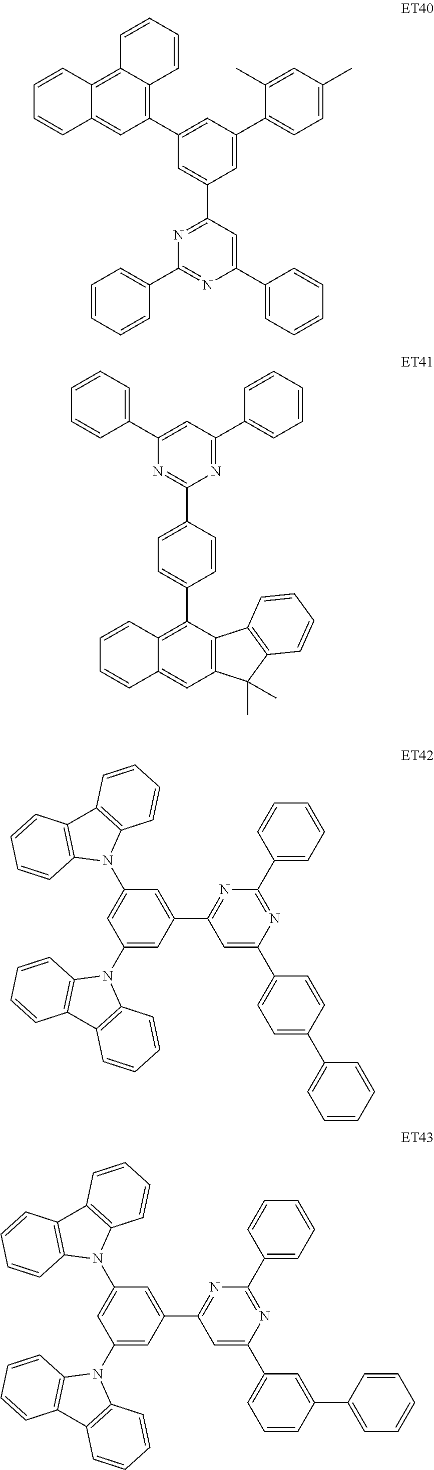

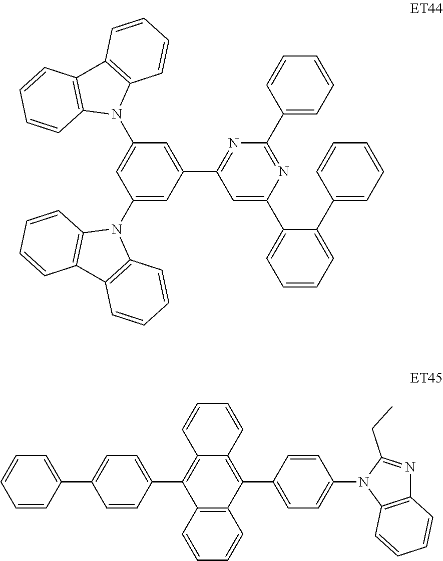 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

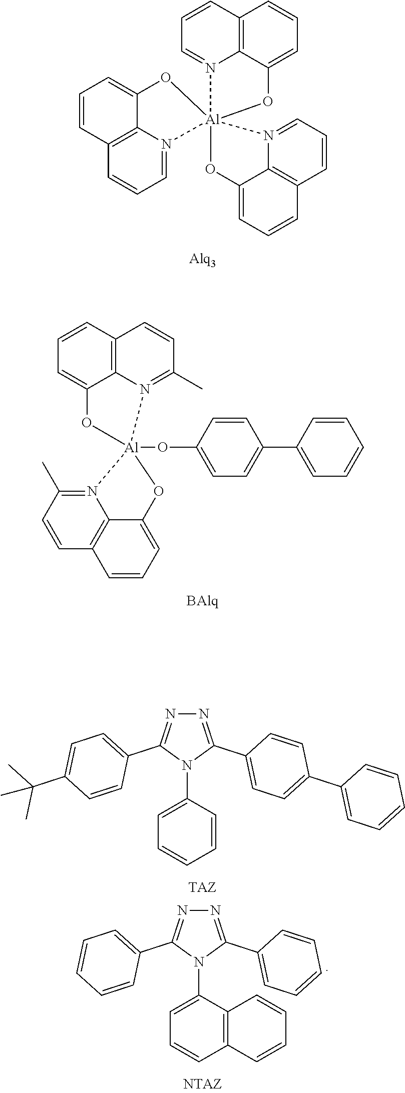

A thickness of the electron transport region may be from about 100 Å to about 5,000 Å, for example, about 160 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be from about 20 Å to about 1,000 Å, for example, about 30 Å to about 300 Å, and a thickness of the electron transport layer may be from about 100 Å to about 1,000 Å, for example, about 150 Å to about 500 Å. When the thicknesses of the buffer layer, the hole blocking layer, the electron control layer, the electron transport layer, and/or the electron transport region are within these ranges, satisfactory electron transporting characteristics may be obtained without a substantial increase in driving voltage.

In one or more embodiments, the electron transport region (for example, the electron transport layer in the electron transport region) may further include, in addition to one or more of the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, in some embodiments, the metal-containing material may include a Li complex. The Li complex may include, for example, Compound ET-D1 (LiQ) or ET-D2:

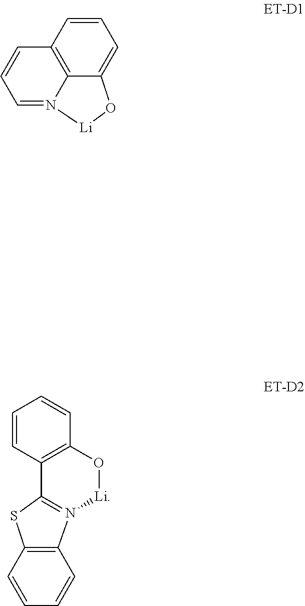

In one or more embodiments, the electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, respectively, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal tellurides. Non-limiting examples of the lanthanide metal telluride may be LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion (i.e., the respective metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, in some embodiments, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be substantially uniformly or non-uniformly dispersed in a matrix including the organic material.

A thickness of the electron injection layer may be in a range of about 1 Å to about 100 Å, or, for example, about 3 Å to about 90 Å. When the thickness of the electron injection layer is within the ranges described above, satisfactory electron injection characteristics may be obtained without a substantial increase in driving voltage.

### Second Electrode 150

The second electrode 150 may be located on the interlayer 130 having a structure as described above. The second electrode 150 may be a cathode, which is an electron injection electrode, and as a material for the second electrode 150, a metal, an alloy, an electrically conductive compound, or any combination thereof, each having a low-work function, may be utilized.

The second electrode 150 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be located outside (e.g., on) the first electrode 110, and/or a second capping layer may be located outside (e.g., on) the second electrode 150. In one or more embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In some embodiments, light generated in the emission layer of the interlayer 130 of the light-emitting device 10 may be extracted toward the outside through the first electrode 110 which is a semi-transmissive electrode or a transmissive electrode, and the first capping layer. In some embodiments, light generated in the emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may (e.g., the first capping layer and the second capping layer may each independently) include carbocyclic compounds, heterocyclic compounds, amine group-containing compounds, porphine derivatives, phthalocyanine derivatives, naphthalocyanine derivatives, alkali metal complexes, alkaline earth metal complexes, or any combination thereof. Optionally, the carbocyclic compound, the heterocyclic compound, and the amine group-containing compound may each be substituted with a substituent including O, N, S, Se, Si, F, Cl, Br, I, or any combination thereof. In one or more embodiments, at least one of the first capping layer or the second capping layer may (e.g., the first capping layer and the second capping layer may each independently) include an amine group-containing compound, e.g., the amine-containing compound of one or more embodiments of the present disclosure.

For example, in one or more embodiments, at least one of the first capping layer or the second capping layer may (e.g., the first capping layer and the second capping layer may each independently) include a compound represented by Formula 201, a compound represented by Formula 202, or any combination thereof.

In one or more embodiments, at least one of the first capping layer or the second capping layer may (e.g., the first capping layer and the second capping layer may each independently) include at least one selected from among Compounds HT28 to HT33, at least one selected from among Compounds CP1 to CP6, β-NPB, or any combination thereof:

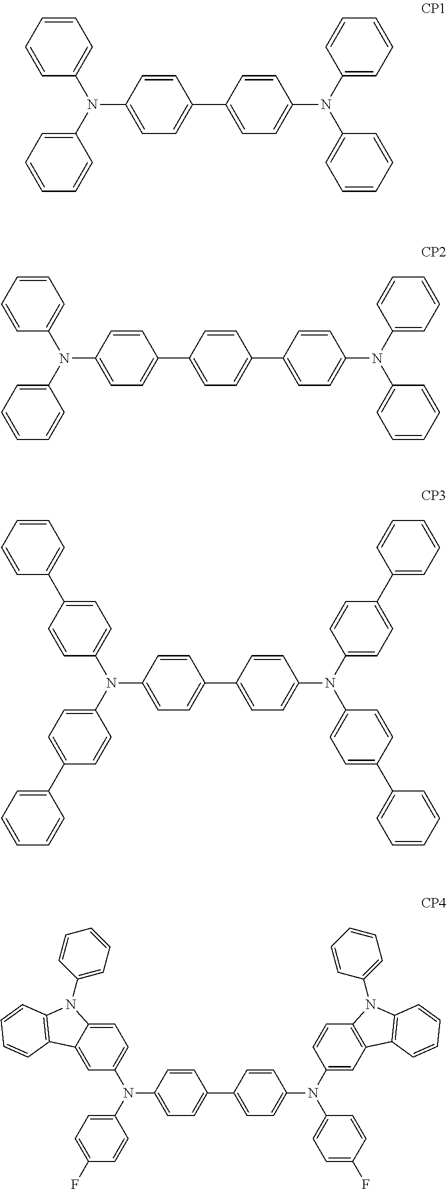 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

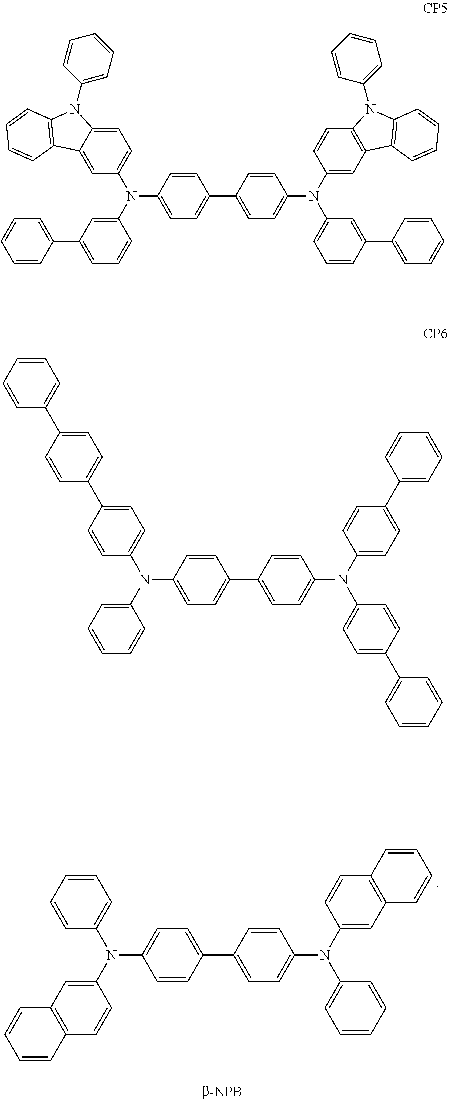 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The amine-containing compound represented by Formula 1 may be included in one or more suitable films. Accordingly, one or more aspects of embodiments of the present disclosure are directed toward a film including the amine-containing compound represented by Formula 1. The film may be, for example, an optical member (or a light control element) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, and/or the like), and/or a protective member (for example, an insulating layer, a dielectric layer, and/or the like).

### Electronic Apparatus

The light-emitting device may be included in one or more suitable electronic apparatuses. For example, the electronic apparatus including the light-emitting device may be a light-emitting apparatus, an authentication apparatus, and/or the like.

In one or more embodiments, the electronic apparatus (for example, a light-emitting apparatus) may further include, in addition to the light-emitting device, i) a color filter, ii) a color conversion layer, or iii) a color filter and a color conversion layer. The color filter and/or the color conversion layer may be located in at least one direction in which light emitted from the light-emitting device travels. For example, in some embodiments, the light emitted from the light-emitting device may be blue light or white light (e.g., combined white light). For details on the light-emitting device, related description provided above may be referred to. In one or more embodiments, the color conversion layer may include a quantum dot. The quantum dot may be, for example, a quantum dot described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or the plurality of color conversion areas) may include a first area configured to emit first color light, a second area configured to emit second color light, and/or a third area configured to emit third color light, wherein the first color light, the second color light, and/or the third color light may have different maximum emission wavelengths from one another. For example, in one or more embodiments, the first color light may be red light, the second color light may be green light, and the third color light may be blue light. For example, in one or more embodiments, the plurality of color filter areas (or the plurality of color conversion areas) may include quantum dots. In some embodiments, the first area may include a red quantum dot to emit red light, the second area may include a green quantum dot to emit green light, and the third area may not include (e.g., may exclude) any quantum dot. For details on the quantum dot, related descriptions provided herein may be referred to. The first area, the second area, and/or the third area may each include a scatter.

For example, in one or more embodiments, the light-emitting device may be to emit first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum emission wavelengths. In some embodiments, the first light may be blue light, the first-first color light may be red light, the second-first color light may be green light, and the third-first color light may be blue light.

In one or more embodiments, the electronic apparatus may further include a thin-film transistor, in addition to the light-emitting device as described above. The thin-film transistor may include a source electrode, a drain electrode, and an activation layer, wherein one selected from the source electrode and the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be located between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally located on the sealing portion, in addition to the color filter and/or the color conversion layer, according to the utilization of the electronic apparatus. Non-limiting examples of the functional layers may include a touch screen layer, a polarizing layer, and/or the like. The touch screen layer may be a pressure-sensitive touch screen layer, a capacitive touch screen layer, or an infrared touch screen layer.

The authentication apparatus may be, for example, a biometric authentication apparatus that authenticates an individual by utilizing biometric information of a living body (for example, fingertips, pupils, etc.). The authentication apparatus may further include, in addition to the light-emitting device as described above, a biometric information collector.

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device may be included in one or more suitable electronic equipment.

For example, the electronic equipment including the light-emitting device may be at least one of a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater or stadium screen, a phototherapy device, or a signboard.

The light-emitting device may have excellent or suitable effects in terms of luminescence efficiency and long lifespan, and thus the electronic equipment including the light-emitting device may have desirable characteristics, such as high luminance, high resolution, and low power consumption.

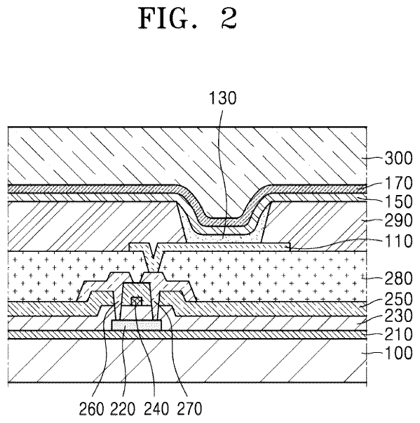

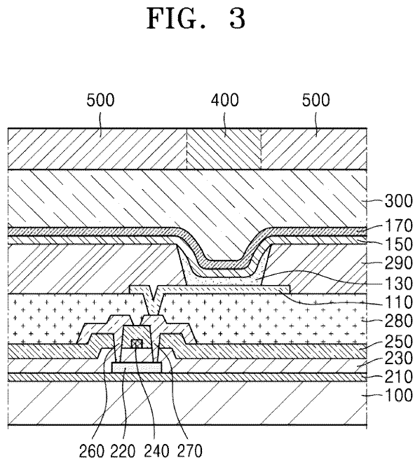

### Description of FIG. 2 and FIG. 3

FIG. 2 is a cross-sectional view showing a light-emitting apparatus according to one or more embodiments of the present disclosure.

The light-emitting apparatus of FIG. 2 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100 and may provide a flat surface on the substrate 100.

The TFT may be on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be located between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220, respectively.

The TFT is electrically connected to the light-emitting device to drive the light-emitting device, and is covered and protected by a passivation layer 280. The passivation layer 280 may include an inorganic insulating film, an organic insulating film, or any combination thereof. The light-emitting device is provided on the passivation layer 280. The light-emitting device may include a first electrode 110, an interlayer 130, and a second electrode 150.

The first electrode 110 may be on the passivation layer 280. The passivation layer 280 may be located to expose a portion of the drain electrode 270, not fully covering the drain electrode 270, and the first electrode 110 may be located to be connected to the exposed portion of the drain electrode 270.

A pixel defining layer 290 including an insulating material may be on the first electrode 110. The pixel defining layer 290 may expose a certain region of the first electrode 110, and the interlayer 130 may be formed in the exposed region of the first electrode 110. The pixel defining layer 290 may be a polyimide or polyacrylic organic film. In some embodiments, at least some layers of the interlayer 130 may extend beyond the upper portion of the pixel defining layer 290 to be located in the form of a common layer.

The second electrode 150 may be on the interlayer 130, and a capping layer 170 may be additionally formed on the second electrode 150. The capping layer 170 may be formed to cover the second electrode 150.

The encapsulation portion 300 may be on the capping layer 170. The encapsulation portion 300 may be located on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 shows a cross-sectional view showing a light-emitting apparatus according to one or more embodiments of the present disclosure.

The light-emitting apparatus of FIG. 3 is substantially the same as the light-emitting apparatus of FIG. 2, except that a light-shielding pattern 500 and a functional region 400 are additionally located on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 3 may be a tandem light-emitting device.

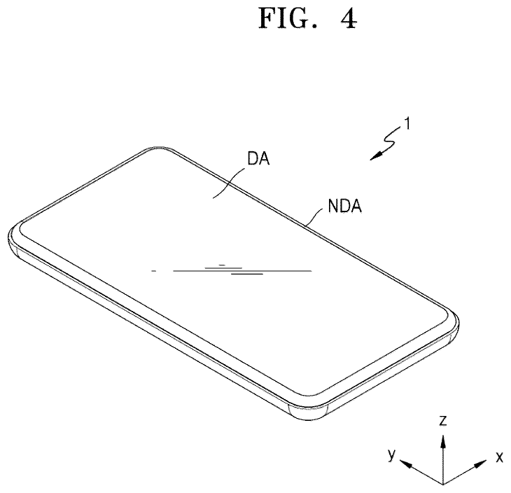

### Description of FIG. 4

FIG. 4 is a schematic perspective view of electronic equipment 1 including a light-emitting device according to one or more embodiments. The electronic equipment 1 may be, as a device apparatus that displays a moving image or still image, a portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC), as well as one or more suitable products, such as a television, a laptop, a monitor, a billboard, or an Internet of things (IOT). The electronic equipment 1 may be such a product above or a part thereof. In some embodiments, the electronic equipment 1 may be a wearable device, such as a smart watch, a watch phone, a glasses-type or kind display, or a head mounted display (HMD), or a part of the wearable device. However, embodiments of the disclosure are not limited thereto. For example, the electronic equipment 1 may be a center information display (CID) on an instrument panel and a center fascia or dashboard of a vehicle, a room mirror display instead of a side mirror of a vehicle, an entertainment display for the rear seat of a vehicle or a display placed on the back of the front seat thereof, head up display (HUD) installed in the front of a vehicle or projected on a front window glass, and/or a computer generated hologram augmented reality head up display (CGH AR HUD). FIG. 4 illustrates an embodiment in which the electronic equipment 1 is a smartphone for convenience of explanation.

The electronic equipment 1 may include a display area DA and a non-display area NDA outside the display area DA. A display device of the electronic equipment 1 may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

In the electronic equipment 1, a length in the x-axis direction and a length (e.g., a width) in the y-axis direction may be different from each other. For example, as shown in FIG. 4, in some embodiments, the length in the x-axis direction may be shorter than the length (e.g., the width) in the y-axis direction. In one or more embodiments, the length in the x-axis direction may be the same as the length (e.g., the width) in the y-axis direction. In one or more embodiments, the length in the x-axis direction may be longer than the length (e.g., the width) in the y-axis direction.

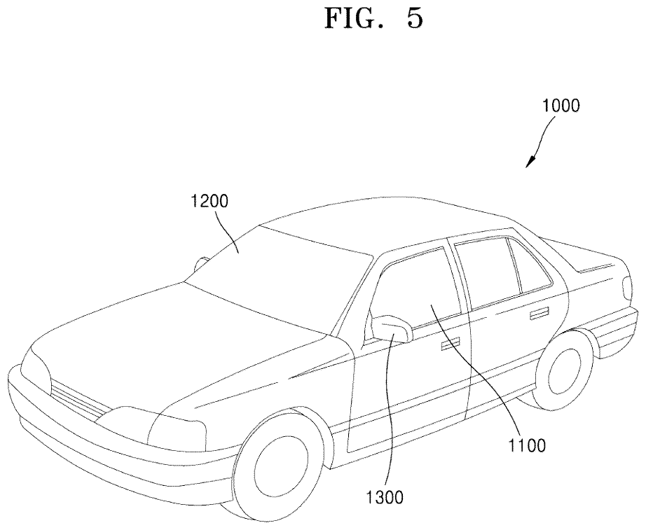

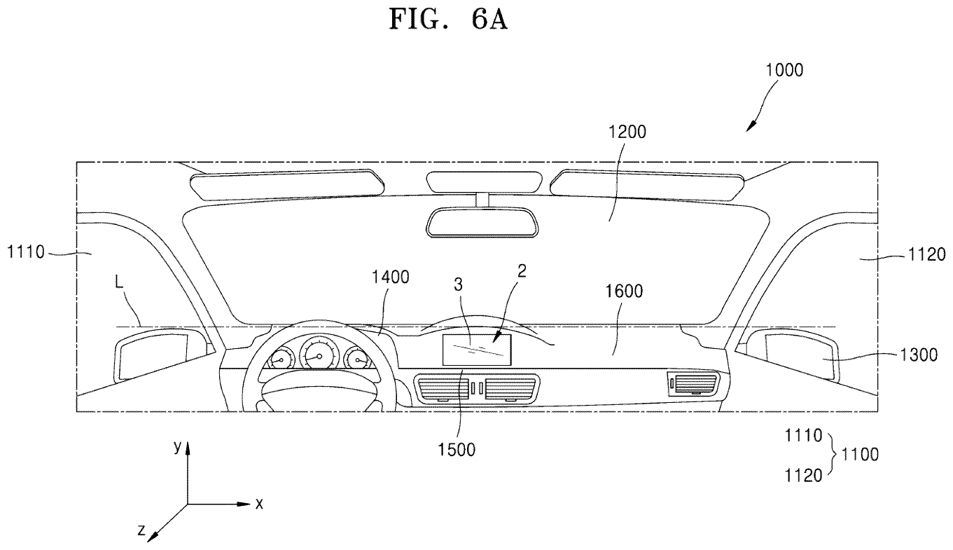

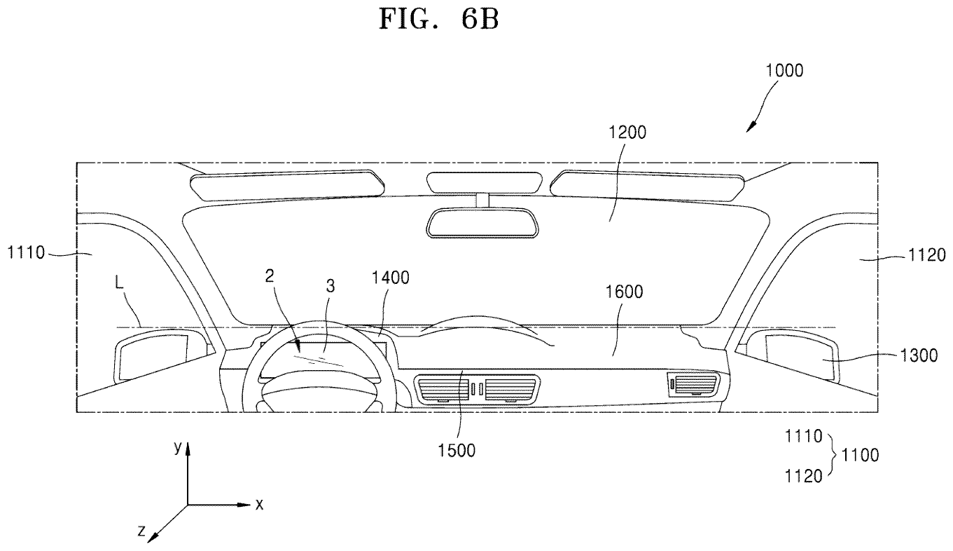

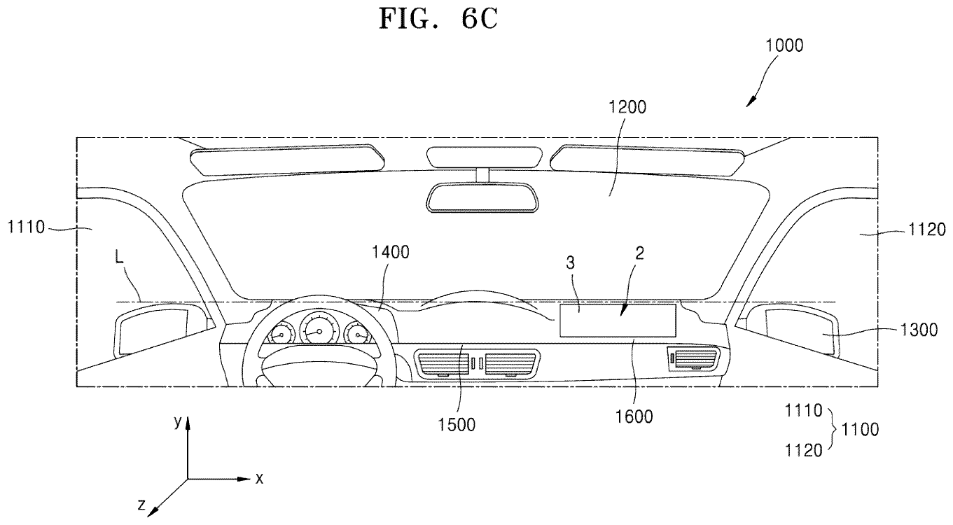

### Description of FIGS. 5 and 6A to 6C

FIG. 5 is a schematic view of an exterior of a vehicle 1000 as electronic equipment including a light-emitting device according to one or more embodiments.

FIGS. 6A to 6C are each a schematic view of an interior of the vehicle 1000 according to one or more embodiments.

Referring to FIGS. 5, 6A, 6B, and 6C, the vehicle 1000 may refer to one or more suitable apparatuses for moving an object to be transported, such as a human, an object, or an animal, from a departure point to a destination. The vehicle 1000 may include a vehicle traveling on a road or a track, a vessel moving over a sea or a river, an airplane flying in the sky utilizing the action of air, and/or the like.

In one or more embodiments, the vehicle 1000 may travel on a road or a track. The vehicle 1000 may move in a set or predetermined direction according to the rotation of at least one wheel thereof. For example, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on a side of the vehicle 1000. In some embodiments, the side window glass 1100 may be installed on a door of the vehicle 1000. A plurality of side window glasses 1100 may be provided and may face each other. In some embodiments, the side window glass 1100 may include a first side window glass 1110 and a second side window glass 1120. In some embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the x-direction or the −x-direction (the direction opposite the x-direction). For example, in some embodiments, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In some embodiments, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning light, a seat belt warning light, an odometer, a hodometer, an automatic shift selector indicator, a door open warning light, an engine oil warning light, and/or a low fuel warning light.

The center fascia 1500 may include a control panel on which a plurality of buttons for adjusting an audio device, an air conditioning device, and/or a heater of a seat are disposed. The center fascia 1500 may be arranged on one side of the cluster 1400.

The passenger seat dashboard 1600 may be spaced apart from the cluster 1400 with the center fascia 1500 arranged therebetween. In some embodiments, the cluster 1400 may be arranged to correspond to a driver seat, and the passenger seat dashboard 1600 may be disposed to correspond to a passenger seat. In some embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In some embodiments, the display device 2 may be arranged between the side window glasses 1100 facing each other. The display device 2 may be arranged on at least one of the cluster 1400, the center fascia 1500, or the passenger seat dashboard 1600.

The display device 2 may include an organic light-emitting display device, an inorganic electroluminescent (EL) display device, a quantum dot display device, and/or the like. Hereinafter, as the display device 2 according to one or more embodiments of the disclosure, an organic light-emitting display device including the light-emitting device according to the disclosure will be described as an example, but one or more suitable types (kinds) of display devices as described above may be utilized in embodiments of the disclosure.

Referring to FIG. 6A, in one or more embodiments, the display device 2 may be arranged on the center fascia 1500. In some embodiments, the display device 2 may display navigation information. In some embodiments, the display device 2 may display audio, video, or information regarding vehicle settings.

Referring to FIG. 6B, in one or more embodiments, the display device 2 may be arranged on the cluster 1400. When the display device 2 is arranged on the cluster 1400, the cluster 1400 may display driving information and/or the like through the display device 2. For example, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, in one or more embodiments, the display device 2 may be arranged on the passenger seat dashboard 1600. The display device 2 may be embedded in the passenger seat dashboard 1600 or arranged on the passenger seat dashboard 1600. In some embodiments, the display device 2 arranged on the passenger seat dashboard 1600 may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may each be formed in a certain region by utilizing one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are each formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group including (e.g., consisting of) carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group including (e.g., consisting of) one (e.g., only one) ring or a polycyclic group in which two or more rings are condensed with each other. For example, the C₁-C₆₀ heterocyclic group has 3 to 61 ring-forming atoms.

The term “cyclic group” as utilized herein may include the C₃-C₆₀ carbocyclic group, and the C₁-C₆₀ heterocyclic group.

The term “π electron-rich C₃-C₆₀ cyclic group” as utilized herein refers to a cyclic group that has three to sixty carbon atoms and does not include *—N=*′ as a ring-forming moiety, and the term “π electron-deficient nitrogen-containing C₁-C₆₀ cyclic group” as utilized herein refers to a heterocyclic group that has one to sixty carbon atoms and includes *—N=*′ as a ring-forming moiety.

For example, the C₃-C₆₀ carbocyclic group may be i) Group T1 (e.g., one or more of the groups in Group T1) or ii) a condensed cyclic group in which two or more of Group T1 (e.g., two or more of the groups in Group T1) are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) Group T2, ii) a condensed cyclic group in which two or more of Group T2 are condensed with each other, or iii) a condensed cyclic group in which at least one Group T2 (e.g., at least one of the groups in Group T2) and at least one Group T1 (e.g., at least one of the groups in Group T1) are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

Group T3 may be a furan group, a thiophene group, a 1H-pyrrole group, a silole group, or a borole group, and

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as utilized herein may refer to a group condensed to any cyclic group, a monovalent group, or a polyvalent group (for example, a divalent group, a trivalent group, a tetravalent group, etc.) according to the structure of a formula for which the corresponding term is utilized. In one or more embodiments, “a benzene group” may be a benzo group, a phenyl group, a phenylene group, and/or the like, which may be easily understand by one of ordinary skill in the art according to the structure of a formula including the “benzene group.”

Depending on context, in the present disclosure, a divalent group may refer or be a polyvalent group (e.g., trivalent, tetravalent, etc., and not just divalent) per, e.g., the structure of a formula in connection with which of the terms are utilized.

Non-limiting examples of the monovalent C₃-C₆₀ carbocyclic group and the monovalent C₁-C₆₀ heterocyclic group may be a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₃-C₆₀ carbocyclic group and the divalent C₁-C₆₀ heterocyclic group may be a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has one to sixty carbon atoms, and non-limiting examples thereof may be a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having substantially the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of a C₂-C₆₀ alkyl group, and non-limiting examples thereof may be an ethenyl group, a propenyl group, and a butenyl group. The term “C₂-C₆₀ alkenylene group” as utilized herein refers to a divalent group having substantially the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of a C₂-C₆₀ alkyl group, and non-limiting examples thereof may include an ethynyl group, a propynyl group, and/or the like. The term “C₂-C₆₀ alkynylene group” as utilized herein refers to a divalent group having substantially the same structure as the C₂-C₆₀ alkynyl group.

The term “C₁-C₆₀ alkoxy group” as utilized herein refers to a monovalent group represented by—OA₁₀₁ (wherein A₁₀₁ is a C₁-C₆₀ alkyl group), and non-limiting examples thereof may include a methoxy group, an ethoxy group, and an isopropyloxy group.

The term “C₃-C₁₀ cycloalkyl group” as utilized herein refers to a monovalent saturated hydrocarbon cyclic group having 3 to 10 carbon atoms, and non-limiting examples thereof may be a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as utilized herein refers to a divalent group having substantially the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and non-limiting examples may be a 1,2,3,4-oxatriazolidinyl group, a tetrahydrofuranyl group, and a tetrahydrothiophenyl group. The term “C₁-C₁₀ heterocycloalkylene group” as utilized herein refers to a divalent group having substantially the same structure as the C₁-C₁₀ heterocycloalkyl group.

The term C₃-C₁₀ cycloalkenyl group utilized herein refers to a monovalent cyclic group that has three to ten carbon atoms and at least one carbon-carbon double bond in the ring thereof and no aromaticity, and non-limiting examples thereof may be a cyclopentenyl group, a cyclohexenyl group, and a cycloheptenyl group. The term “C₃-C₁₀ cycloalkenylene group” as utilized herein refers to a divalent group having substantially the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one double bond in the cyclic structure thereof. Non-limiting examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having substantially the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Non-limiting examples of the C₆-C₆₀ aryl group may be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Non-limiting examples of the C₁-C₆₀ heteroaryl group may be a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure as a whole. Non-limiting examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having substantially the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure as a whole. Non-limiting examples of the monovalent non-aromatic condensed heteropolycyclic group may be a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having substantially the same structure as the monovalent non-aromatic condensed heteropolycyclic group described above.

The term “C₆-C₆₀ aryloxy group” as utilized herein indicates—OA₁₀₂ (wherein A₁₀₂ is a C₆-C₆₀ aryl group), and the term “C₆-C₆₀ arylthio group” as utilized herein indicates—SA₁₀₃ (wherein A₁₀₃ is a C₆-C₆₀ aryl group).

The term “C₇-C₆₀ arylalkyl group” utilized herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroarylalkyl group” utilized herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as utilized herein refers to:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁—Co alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂—C o heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂—Co alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Non-limiting examples of the heteroatom may be O, S, N, P, Si, B, Ge, Se, and/or one or more combinations thereof.

The term “third-row transition metal” utilized herein includes hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “Bu^{t}” as utilized herein refers to a tert-butyl group, and the term “OMe” as utilized herein refers to a methoxy group.

The term “biphenyl group” as utilized herein refers to “a phenyl group substituted with a phenyl group.” In some embodiments, the “biphenyl group” is a substituted phenyl group having a C₆-C₆₀ aryl group as a substituent.

The term “terphenyl group” as utilized herein refers to “a phenyl group substituted with a biphenyl group”. In some embodiments, the “terphenyl group” is a substituted phenyl group having, as a substituent, a C₆-C₆₀ aryl group substituted with a C₆-C₆₀ aryl group.

* and *′ as utilized herein, unless defined otherwise, each refer to a binding site to a neighboring atom in a corresponding formula or moiety.

In the present disclosure, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to one or more embodiments and light-emitting devices according to one or more embodiments will be described in more detail with reference to the following Synthesis Examples and Examples. The wording “B was utilized instead of A” utilized in describing Synthesis Examples refers to that an identical molar equivalent of B was utilized in place of A.

### EXAMPLES

### Synthesis Example 1 (Compound 30)

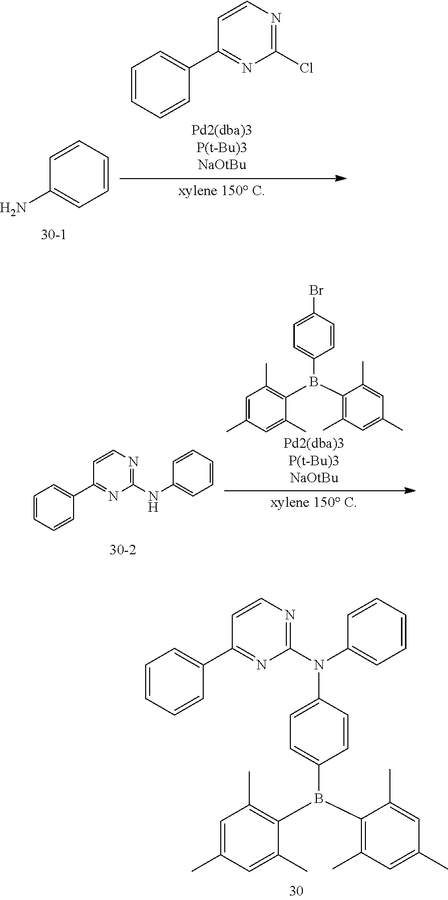

### Synthesis of Intermediate 30-2

Intermediate 30-1 (0.93 g), 2-chloro-4-phenylpyrimidine (1.91 g), tris(dibenzylideneacetone)dipalladium(0) (Pd₂(dba)₃) (0.46 g), tri-tert-butylphosphine (P(t-Bu)₃) (0.21 g), and sodium tert-butoxide (NaOtBu) (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 30-2 (1.81 g, yield: 73%).

### Synthesis of Compound 30

Intermediate 30-2 (2.47 g), (4-bromophenyl)dimesitylborane (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 30 (4.45 g, yield: 78%).

### Synthesis Example 2 (Compound 95)

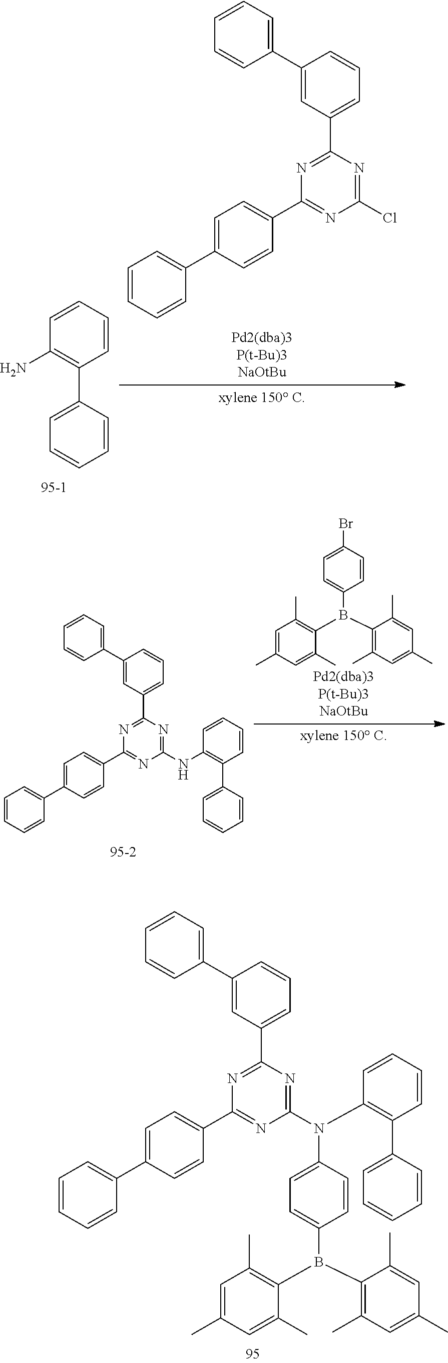

### Synthesis of Intermediate 95-2

Intermediate 95-1 (1.69 g), 2-([1,1′-biphenyl]-3-yl)-4-([1,1′-biphenyl]-4-yl)-6-chloro-1,3,5-triazine (4.19 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 95-2 (4.41 g, yield: 80%).

### Synthesis of Compound 95

Intermediate 95-2 (5.52 g), (4-bromophenyl)dimesitylborane (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 95 (6.30 g, yield: 72

### Synthesis Example 3 (Compound 179)

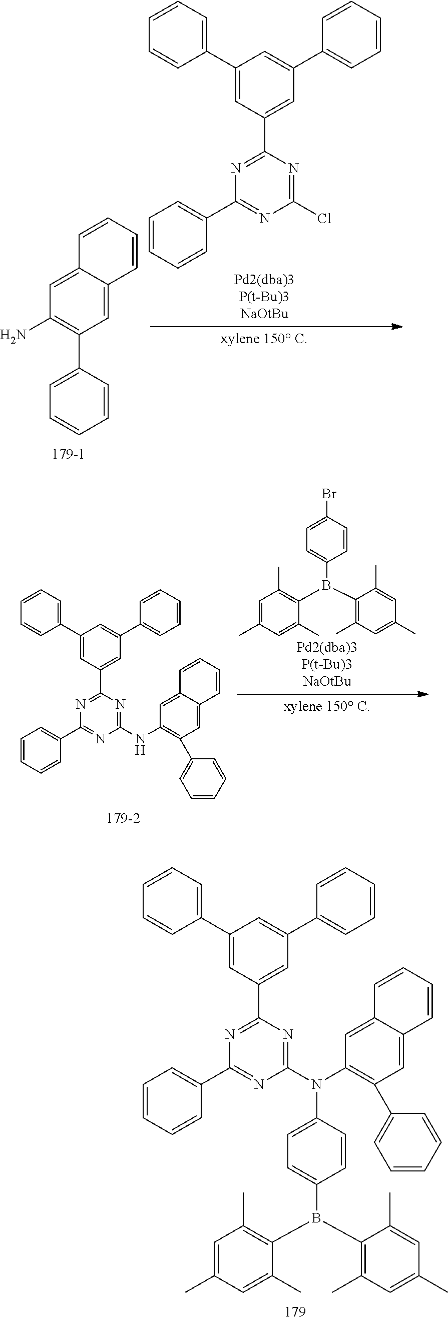

### Synthesis of Intermediate 179-2

Intermediate 179-1 (2.19 g), 2-([1,1′: 3′, 1″-terphenyl]-5′-yl)-4-chloro-6-phenyl-1,3,5-triazine (4.19 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 179-2 (4.63 g, yield: 77%).

### Synthesis of Compound 179

Intermediate 179-2 (6.02 g), (4-bromophenyl)dimesitylborane (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 179 (6.39 g, yield: 69%).

### Synthesis Example 4 (Compound 267)

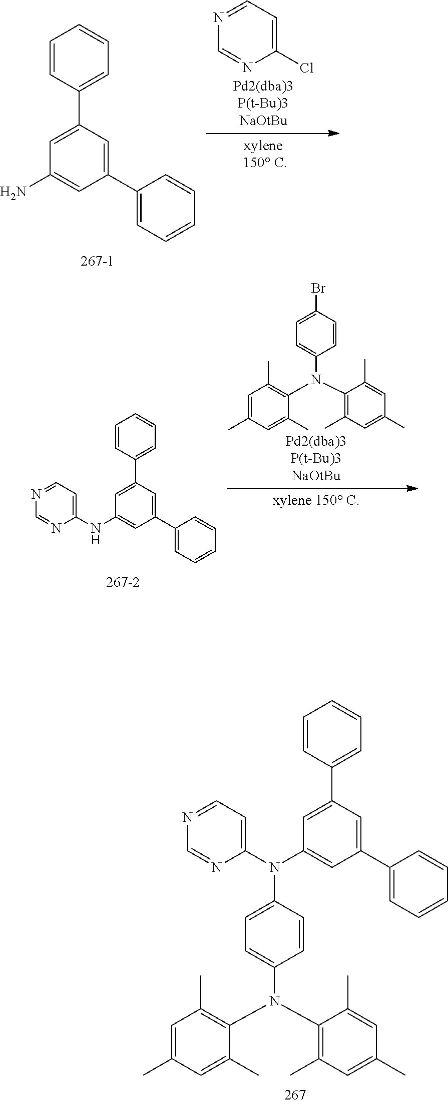

### Synthesis of Intermediate 267-2

Intermediate 267-1 (2.45 g), 4-chloropyrimidine (1.14 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 267-2 (2.68 g, yield: 83%).

### Synthesis of Compound 267

Intermediate 267-2 (3.23 g), N-(4-bromophenyl)-N-mesityl-2,4,6-trim ethylaniline (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 267 (5.52 g, yield: 85%).

### Synthesis Example 5 (Compound 305)

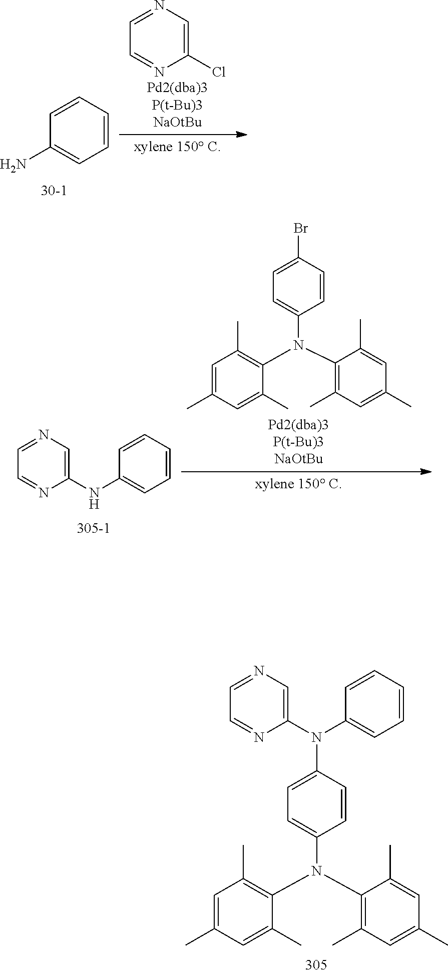

### Synthesis of Intermediate 305-1

Intermediate 30-1 (0.93 g), 2-chloropyrazine (1.14 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 305-1 (1.43 g, yield: 84%).

### Synthesis of Compound 305

Intermediate 305-1 (1.71 g), N-(4-bromophenyl)-N-mesityl-2,4,6-trimethylaniline (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 305 (3.68 g, yield: 74%).

### Synthesis Example 6 (Compound 369)

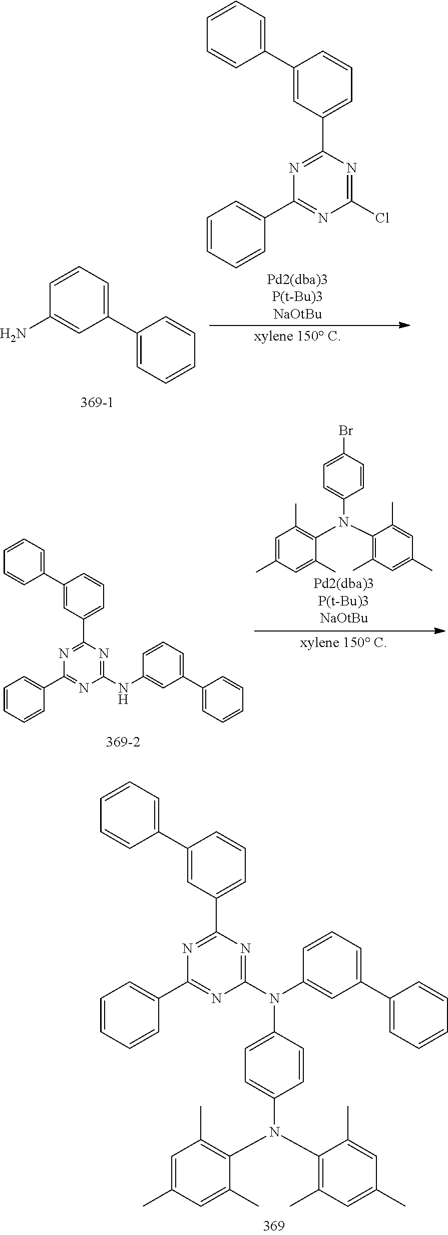

### Synthesis of Intermediate 369-2

Intermediate 369-1 (1.69 g), 2-([1,1′-biphenyl]-3-yl)-4-chloro-6-phenyl-1,3,5-triazine (3.43 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 369-2 (3.38 g, yield: 71%).

### Synthesis of Compound 369

Intermediate 369-2 (4.76 g), N-(4-bromophenyl)-N-mesityl-2,4,6-trimethylaniline (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 369 (5.62 g, yield: 70%).

### Synthesis Example 7 (Compound 462)

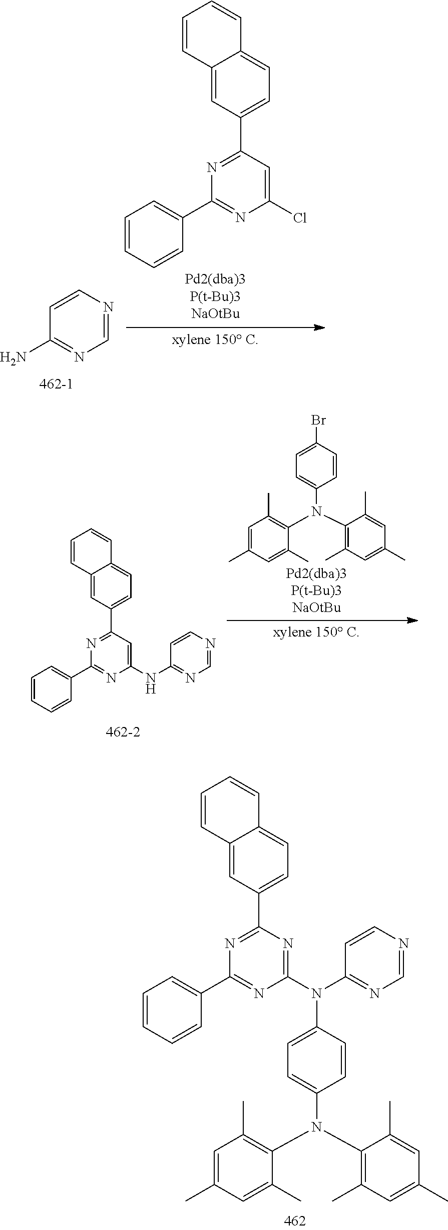

### Synthesis of Intermediate 462-2

Intermediate 462-1 (0.95 g), 4-chloro-6-(naphthalen-2-yl)-2-phenylpyrimidine (3.16 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 462-2 (3.03 g, yield: 81%).

### Synthesis of Compound 462

Intermediate 462-2 (3.75 g), N-(4-bromophenyl)-N-mesityl-2,4,6-trimethylaniline (4.05 g), Pd₂(dba)₃ (0.46 g), P(t-Bu)₃ (0.21 g), and NaOtBu (2.44 g) were dissolved in o-xylene (50 mL) and then stirred for an hour at 150° C. After the 1 reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 462 (5.54 g, yield: 79%).

### Synthesis Example 8 (Compound 521)

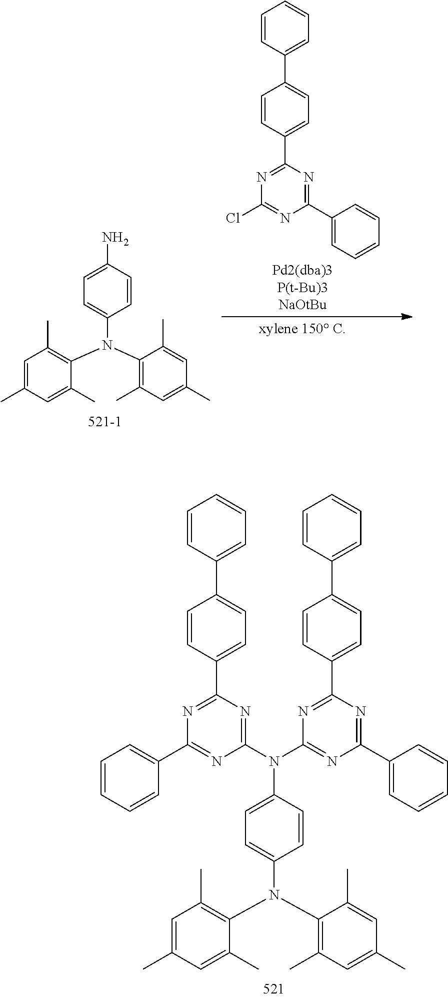

### Synthesis of Compound 521

Intermediate 521-1 (3.44 g), 2-([1,1′-biphenyl]-4-yl)-4-chloro-6-phenyl-1,3,5-triazine (6.86 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 521 (6.41 g, yield: 67%).

### Synthesis Example 9 (Compound 534)

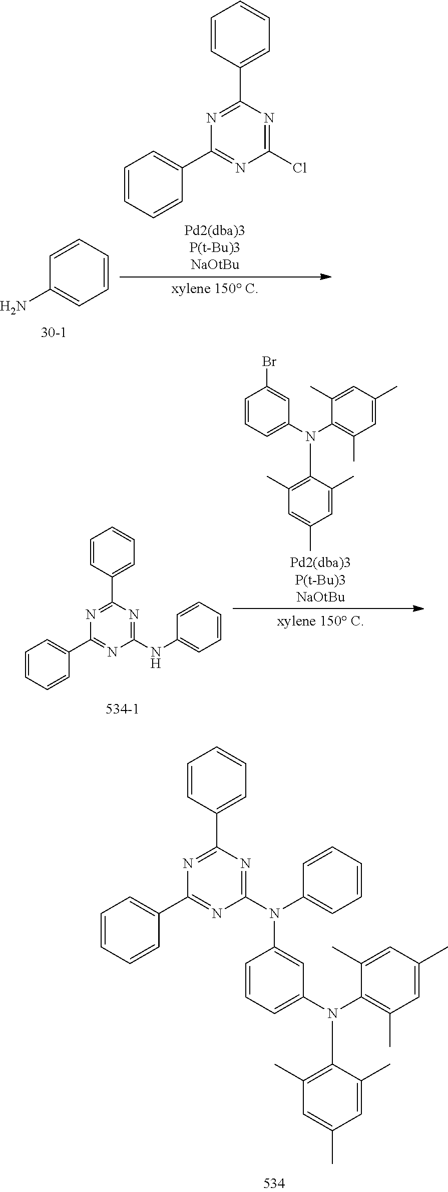 [SMILES: Clc1nc(-c2ccccc2)nc(-c2ccccc2)n1 ; Nc1ccccc1 ; c1ccc(Nc2nc(-c3ccccc3)nc(-c3ccccc3)n2)cc1 ; Cc1cc(C)c(N(c2cccc(Br)c2)c2c(C)cc(C)cc2C)c(C)c1 ; Cc1cc(C)c(N(c2cccc(N(c3ccccc3)c3nc(-c4ccccc4)nc(-c4ccccc4)n3)c2)c2c(C)cc(C)cc2C)c(C)c1]

### Synthesis of Intermediate 534-1

Intermediate 30-1 (0.93 g), 2-chloro-4,6-diphenyl-1,3,5-triazine (2.67 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Intermediate 534-1 (2.26 g, yield: 70%).

### Synthesis of Compound 534

Intermediate 534-1 (3.24 g), N-(3-bromophenyl)-N-mesityl-2,4,6-trimethylaniline (4.08 g), Pd₂(dba)₃ (0.46 g), P(t-Bu)₃ (0.21 g), and NaOtBu (2.44 g) were dissolved in o-xylene (50 mL) and then stirred for an hour at 150° C. After the reaction solution was cooled to room temperature, the reaction was terminated by utilizing water. Then, an extraction process was performed thereon three times by utilizing ethylether. An organic layer extracted therefrom was dried by utilizing anhydrous magnesium sulfate and then distilled under reduced pressure, and a residue thus obtained was separated and purified by column chromatography, thereby obtaining Compound 534 (5.27 g, yield: 81%).

For the compounds synthesized in Synthesis Examples 1 to 9, ¹H NMR and high-resolution mass (HR-MS) were measured, and the results are shown in Table 1. Synthesis methods of compounds other than the compounds of Synthesis Examples 1 to 9 may be readily recognized by those skilled in the art by referring to the synthesis paths and source materials.

TABLE 1

| | | HR-MS (m/z) | |
|---|---|---|---|
| | | [M⁺] | |
| | ¹H NMR (CDCl₃, 500 MHz) | found | calc. |
| Synthesis | 8.09(d, 1H), 7.94(d, 2H), 7.49-7.55(m, 3H), | 570.32 | 571.31 |
| Example 1 | 7.18-7.24(m, 5H), 7.00-7.08 (m, 3H), 6.97 (s, | | |
| | 4H), 2.33 (s, 12H), 2.18 (s, 6H) | | |
| Synthesis | 8.38(d, 1H), 8.10(d, 1H), 7.96(m, 2H), 7.71- | 875.44 | 876.44 |
| Example 2 | 7.75(m, 7H), 7.61 (d, 1H), 7.39-7.49 (m, 11H), | | |
| | 7.08-7.25(m, 7H), 6.97 (s, 4H), 2.33 (s, 12H), | | |
| | 2.18 (s, 6H) | | |
| Synthesis | 8.36(d, 2H), 8.04(s, 3H), 7.86(d, 1H), 7.71- | 925.45 | 926.45 |
| Example 3 | 7.77(m, 8H), 7.41-7.50 (m, 14H), 7.18 (d, 2H), | | |
| | 7.08(d, 2H), 6.97 (s, 4H), 2.33 (s, 12H), 2.18 | | |
| | (s, 6H) | | |
| Synthesis | 8.40(d, 1H), 8.34(s, 1H), 7.75(d, 4H), | 649.34 | 650.34 |
| Example 4 | 7.60(s, 1H), 7.37-7.49 (m, 8H), 7.14 (d, 4H), | | |
| | 6.77(s, 4H), 6.44 (d, 1H), 2.33 (s, 12H), 2.18 | | |
| | (s, 6H) | | |
| Synthesis | 8.35-8.40(m, 3H), 7.24(t, 2H), 7.00-7.14(m, 7H), | 497.28 | 498.28 |
| Example 5 | 6.77(s, 4H), 2.33 (s, 12H), 2.18 (s, 6H) | | |
| Synthesis | 8.36-8.38(m, 3H), 7.94(s, 1H), 7.73- | 802.40 | 803.40 |
| Example 6 | 7.75(m, 4H), 7.41-7.61(m, 11H), 7.27 (s, 1H), | | |
| | 7.14-7.17 (m, 6H), 6.77(s, 4H), 2.33 (s, 12H), | | |
| | 2.18 (s, 6H) | | |
| Synthesis | 8.48(s, 1H), 8.36-8.40(m, 4H), 7.98- | 701.35 | 702.35 |
| Example 7 | 8.06(m, 4H), 7.59-7.61(m, 2H), 7.50 (m, 3H), | | |
| | 7.14(d, 4H), 6.85(s, 1H), 6.77(s, 4H), 6.44(d, | | |
| | 1H), 2.33 (s, 12H), 2.18 (s, 6H) | | |
| Synthesis | 8.36(d, 4H), 7.96(d, 4H), 7.75(d, 4H), 7.41- | 957.45 | 958.45 |
| Example 8 | 7.50(m, 12H), 7.25 (d, 4H), 7.14(d, 4H), | | |
| | 6.77(s, 4H), 2.33 (s, 12H), 2.18 (s, 6H) | | |
| Synthesis | 8.36(d, 4H), 7.50(m, 6H), 7.24(m, 3H), 7.00- | 650.34 | 651.34 |
| Example 9 | 7.08(m, 3H), 6.83 (s, 1H), 6.74-6.77(m, 6H), | | |
| | 2.33 (s, 12H), 2.18 (s, 6H) | | |

### Example 1

As an anode, a Corning 15 Ω/cm² (1,200 Å) ITO glass substrate was cut to a size of 50 mm×50 mm×0.7 mm, sonicated with isopropyl alcohol and pure water each for 5 minutes, and then cleaned by exposure to ultraviolet rays and ozone for 30 minutes. The ITO glass substrate was then provided to a vacuum deposition apparatus.

NPD was deposited on the ITO anode formed on the glass substrate to form a hole injection layer having a thickness of 300 Å, HTL3 was deposited on the hole injection layer to form a hole transport layer having a thickness of 200 Å, and CzSi was deposited on the hole transport layer to form an emission auxiliary layer having a thickness of 100 Å.

A host (HT3 and ET2 at a weight ratio of 1:1), a dopant (t-DABNA), and a sensitizer (PS2) were co-deposited on the emission auxiliary layer at a weight ratio of 84:15:1 to form an emission layer having a thickness of 200 Å.

TSPO1 was deposited on the emission layer to form a hole blocking layer having a thickness of 200 Å, Compound 30 was deposited on the hole blocking layer to form an electron transport layer having a thickness of 300 Å, LiF was deposited on the electron transport layer to form an electron injection layer having a thickness of 10 Å, and then, Al was vacuum-deposited on the electron injection layer to form a LiF/Al electrode (cathode) having a thickness of 3,000 Å.

### Examples 2 to 9 and Comparative Examples 1 to 3

Organic light-emitting devices of Examples 2 to 9 and Comparative Examples 1 to 3 were each manufactured in substantially the same manner as in Example 1, except that components of a host, a dopant, a sensitizer, and an electron transport layer material were respectively adjusted as indicated in Table 2.

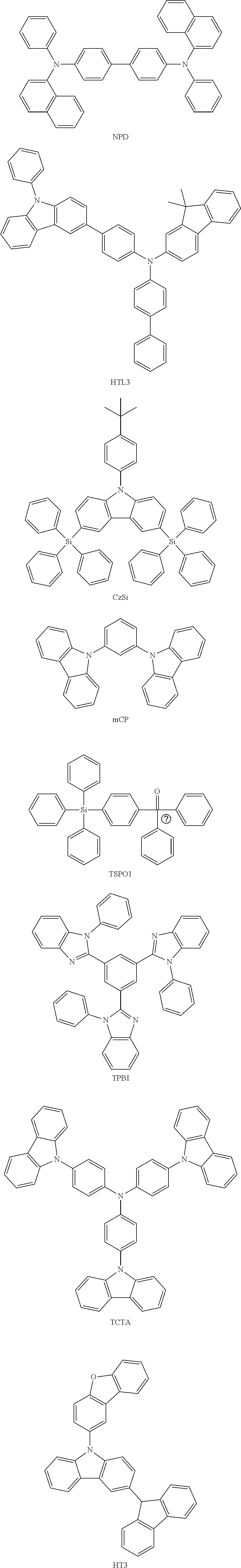

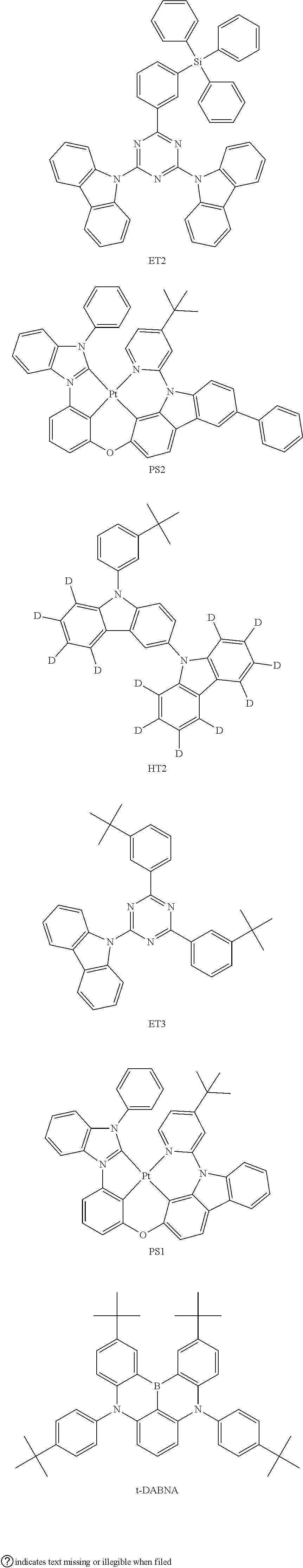

### Evaluation Example 2

To evaluate characteristics of each of the organic light-emitting devices manufactured according to Examples 1 to 9 and Comparative Examples 1 to 3, the driving voltage at a current density of 10 mA/cm² and maximum luminescence efficiency were each measured, and results are shown in Table 2. The driving voltage of each of the organic light-emitting devices was measured by utilizing a source meter (Keithley Instrument Inc., 2400 series), and the maximum luminescence efficiency of each of the organic electroluminescent devices was measured by utilizing the external quantum efficiency measurement apparatus C9920-2-12 of Hamamatsu Photonics Inc. In evaluating the maximum luminescence efficiency, the luminance/current density was measured utilizing a luminance meter that was calibrated for wavelength sensitivity, and the maximum luminescence efficiency was converted by assuming an angular luminance distribution (Lambertian) which introduced a perfect reflecting diffuser. In addition, to evaluate the device lifespan, values obtained by comparing a time taken to reach 95% of the initial luminance in Comparative Example 1 with those of Examples 1 to 9 and Comparative Examples 2 and 3 were calculated.

TABLE 2

| | Hole | Host | | | Electron | Driving | Luminescence | Relative |
|---|---|---|---|---|---|---|---|---|
| | transport | (HT:ET = | | | transport | voltage | efficiency | lifespan |
| | layer | 5:5) | Sensitizer | Dopant | layer | (V) | (cd/A) | (T₉₅) |
| Example 1 | HTL3 | HT3/ET2 | PS2 | t-DABNA | Compound 30 | 4.03 | 25.9 | 4.3 |
| Example 2 | HTL3 | HT3/ET2 | PS2 | t-DABNA | Compound 95 | 4.09 | 26.3 | 4.9 |
| Example 3 | HTL3 | HT3/ET2 | PS2 | t-DABNA | Compound 179 | 3.95 | 26.3 | 5.3 |
| Example 4 | HTL3 | HT3/ET2 | PS2 | t-DABNA | Compound 267 | 4.21 | 25.2 | 5.9 |
| Example 5 | HTL3 | HT3/ET2 | PS2 | t-DABNA | Compound 305 | 4.33 | 25.5 | 4.4 |
| Example 6 | HTL3 | HT3/ET2 | PS2 | t-DABNA | Compound 369 | 4.44 | 25.7 | 3.3 |
| Example 7 | HTL3 | HT3/ET2 | PS2 | t-DABNA | Compound 462 | 4.31 | 26.9 | 6.4 |
| Example 8 | HTL3 | HT2/ET3 | PS1 | t-DABNA | Compound 521 | 4.13 | 25.4 | 4.7 |
| Example 9 | HTL3 | HT2/ET3 | PS1 | t-DABNA | Compound 534 | 4.09 | 25.6 | 4.9 |
| Comparative | HTL3 | HT3/ET2 | PS2 | t-DABNA | Comparative | 5.6 | 8.8 | 1 |
| Example 1 | | | | | Example | | | |
| | | | | | Compound 1 | | | |
| Comparative | HTL3 | HT3/ET2 | PS2 | t-DABNA | Comparative | 4.78 | 23.1 | 2.7 |
| Example 2 | | | | | Example | | | |
| | | | | | Compound 2 | | | |
| Comparative | HTL3 | HT3/ET2 | PS2 | t-DABNA | Comparative | 4.92 | 21.1 | 2.6 |
| Example 3 | | | | | Example | | | |
| | | | | | Compound 3 | | | |

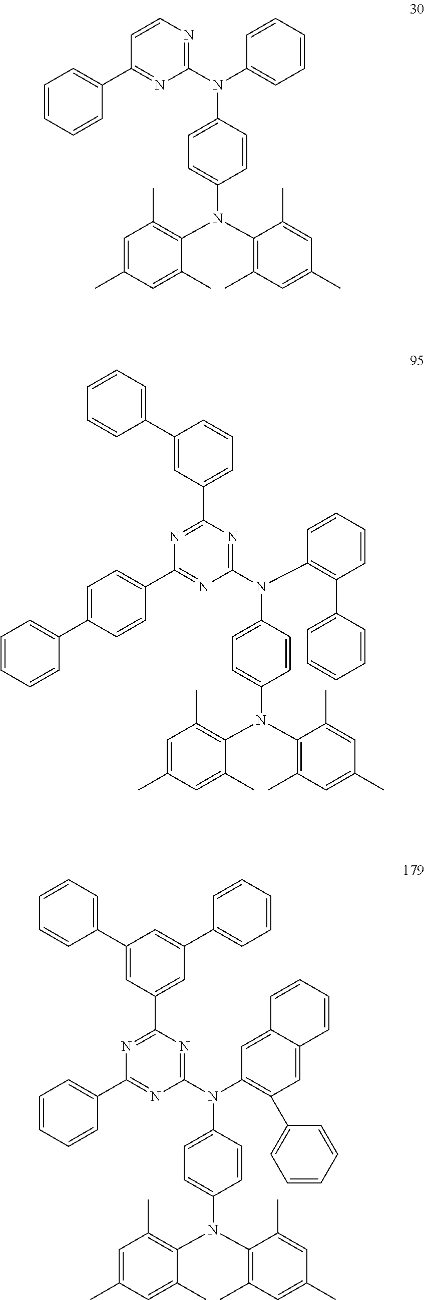 [SMILES: Cc1cc(C)c(N(c2ccc(N(c3ccccc3)c3nccc(-c4ccccc4)n3)cc2)c2c(C)cc(C)cc2C)c(C)c1 ; Cc1cc(C)c(N(c2ccc(N(c3nc(-c4ccc(-c5ccccc5)cc4)nc(-c4cccc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3cc4ccccc4cc3-c3ccccc3)cc2)c2c(C)cc(C)cc2C)c(C)c1]

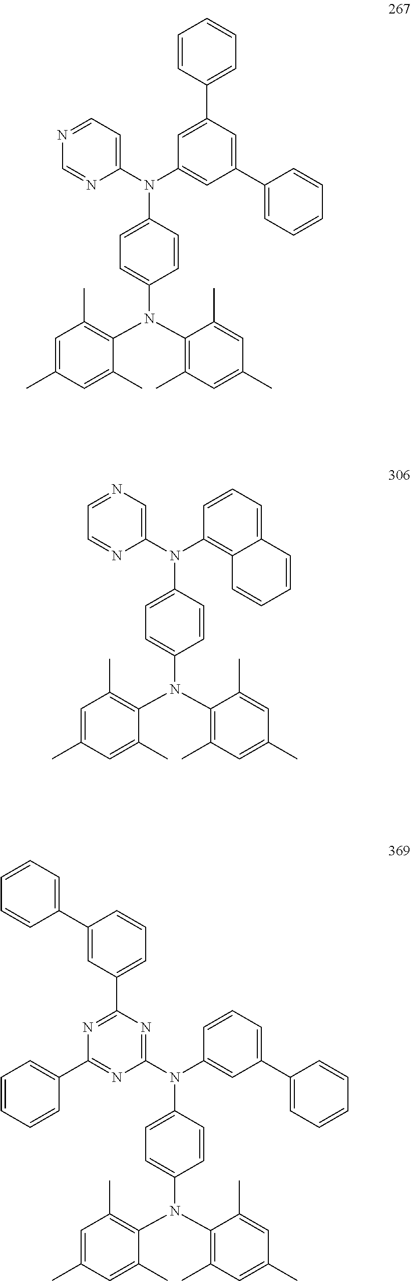

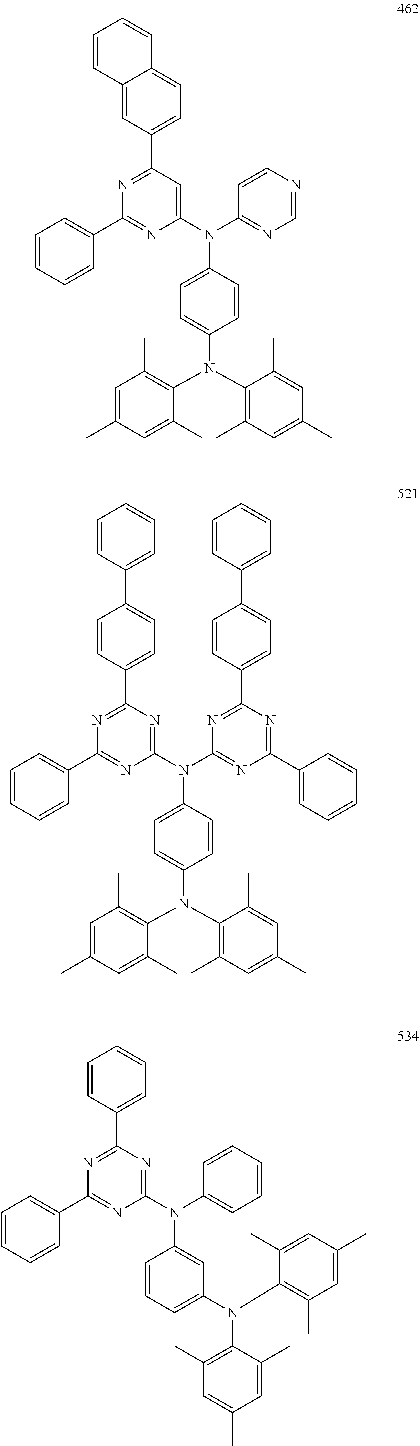 [SMILES: Cc1cc(C)c(N(c2ccc(N(c3ccncn3)c3cc(-c4ccc5ccccc5c4)nc(-c4ccccc4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccc(-c5ccccc5)cc4)n3)cc2)c2c(C)cc(C)cc2C)c(C)c1 ; Cc1cc(C)c(N(c2cccc(N(c3ccccc3)c3nc(-c4ccccc4)nc(-c4ccccc4)n3)c2)c2c(C)cc(C)cc2C)c(C)c1]

### Comparative Example Compound 1 (TPBI)

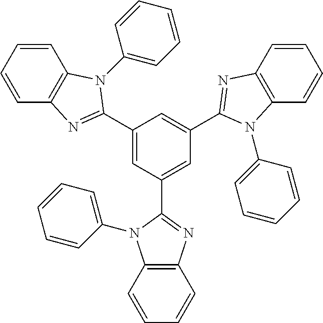 [SMILES: c1ccc(-n2c(-c3cc(-c4nc5ccccc5n4-c4ccccc4)cc(-c4nc5ccccc5n4-c4ccccc4)c3)nc3ccccc32)cc1]

### Comparative Example Compound 2

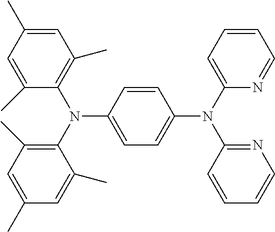 [SMILES: Cc1cc(C)c(N(c2ccc(N(c3ccccn3)c3ccccn3)cc2)c2c(C)cc(C)cc2C)c(C)c1]

### Comparative Example Compound 3

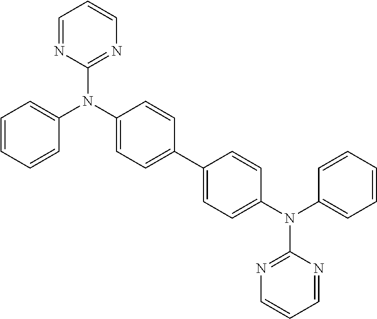 [SMILES: c1ccc(N(c2ccc(-c3ccc(N(c4ccccc4)c4ncccn4)cc3)cc2)c2ncccn2)cc1]

Thus, a light-emitting device having high efficiency and a long lifespan and a high-quality electronic apparatus including the same may be manufactured by utilizing the amine-containing compound.

In the present disclosure, it will be understood that the term “comprise(s),” “include(s),” or “have/has” specifies the presence of stated features, integers, steps, operations, elements, and/or components, but do not preclude the presence or addition of one or more other features, integers, steps, operations, elements, components, and/or groups thereof.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

The light-emitting device, the light-emitting apparatus, the display device, the electronic apparatus, the electronic device, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode;
an interlayer between the first electrode and the second electrode and comprising an emission layer; and
an amine-containing compound represented by Formula 1: 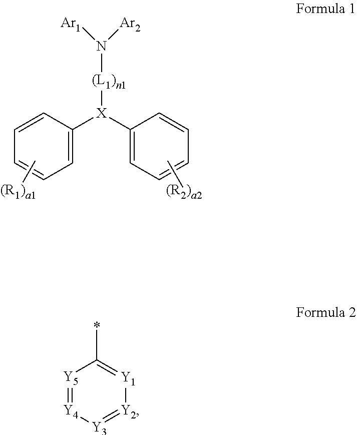
wherein, in Formulae 1 and 2,
X is B or N,
L₁ is a C₃-C₆₀ carbocyclic group or a C₁-C₆₀ heterocyclic group, each unsubstituted or substituted with at least one R₁₀ₐ,
n1 is an integer from 1 to 5,
a1 and a2 are each independently an integer from 1 to 5,
Ar₁ is a group represented by Formula 2,
Ar₂ is a C₆-C₆₀ aryl group or a C₁-C₆₀ heteroaryl group, each unsubstituted or substituted with at least one R₁₀ₐ,
Y₁ is C(R₁₁) or N, Y₂ is C(R₁₂) or N, Y₃ is C(R₁₃) or N, Y₄ is C(R₁₄) or N, Y₅ is C(R₁₅) or N,
at least two selected from among Y₁ to Y₅ are each N,
* indicates a binding site to a neighboring nitrogen atom,
R₁, R₂, and R₁₁ to R₁₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, an electron control layer, or any combination thereof.

3. The light-emitting device of claim 1, wherein the amine-containing compound represented by Formula 1 is in the interlayer.

4. The light-emitting device of claim 2, wherein the amine-containing compound represented by Formula 1 is in the electron transport region.

5. The light-emitting device of claim 2, wherein the amine-containing compound represented by Formula 1 is in the electron transport layer.

6. The light-emitting device of claim 2, further comprising a capping layer on the second electrode,
wherein the amine-containing compound is in the capping layer.

7. An electronic apparatus comprising the light-emitting device of claim 1.

8. The electronic apparatus of claim 7, further comprising a color filter, a color conversion layer, a touch screen layer, a polarizing layer, or any combination thereof.

9. An electronic equipment comprising the light-emitting device of claim 1.

10. The electronic equipment of claim 9, wherein the electronic equipment is at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, or a signboard.

11. An amine-containing compound represented by Formula 1: 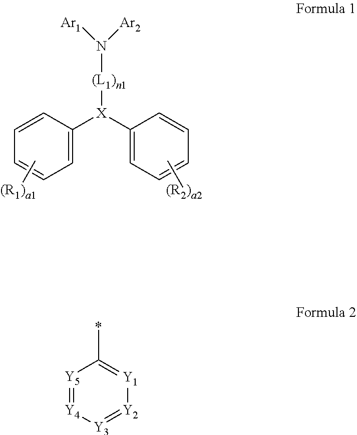
wherein, in Formulae 1 and 2,
X is B or N,
L₁ is a C₃-C₆₀ carbocyclic group or a C₁-C₆₀ heterocyclic group, each unsubstituted or substituted with at least one R₁₀ₐ,
n1 is an integer from 1 to 5,
a1 and a2 are each independently an integer from 1 to 5,
Ar₁ is a group represented by Formula 2,
Ar₂ is a C₆-C₆₀ aryl group or a C₁-C₆₀ heteroaryl group, each unsubstituted or substituted with at least one R₁₀ₐ,
Y₁ is C(R₁₁) or N, Y₂ is C(R₁₂) or N, Y₃ is C(R₁₃) or N, Y₄ is C(R₁₄) or N, Y₅ is C(R₁₅) or N,
at least two selected from among Y₁ to Y₅ are each N,
* indicates a binding site to a neighboring nitrogen atom,
R₁, R₂, and R₁₁ to R₁₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂—Co heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

12. The amine-containing compound of claim 11, wherein Ar₁ and Ar₂ are identical to each other.

13. The amine-containing compound of claim 11, wherein Ar and Ar₂ are different from each other.

14. The amine-containing compound of claim 11, wherein Formula 2 is represented by one selected from among Formulae 2-1 to 2-4: 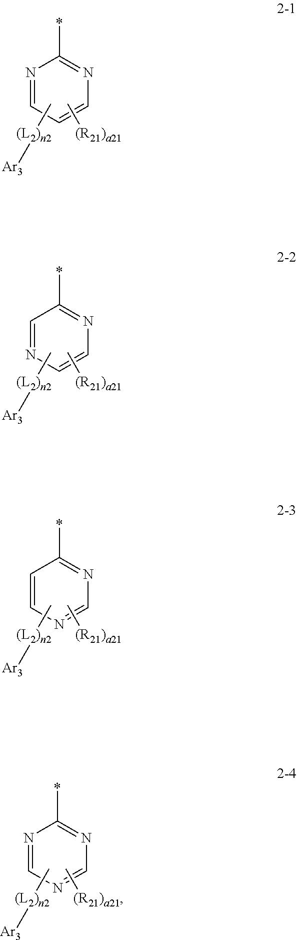
and
wherein, in Formulae 2-1 to 2-4,
L₂ is the same as defined with respect to L₁ in Formula 1,
R₂₁ is the same as defined with respect to R₁ in Formula 1,
Ar₃ is the same as defined with respect to Ar₂ in Formula 1,
a21 is an integer from 1 to 3, and
n2 is an integer from 0 to 4.

15. The amine-containing compound of claim 11, wherein L₁ is a group represented by any one selected from among Formulae 3-1 to 3-6: 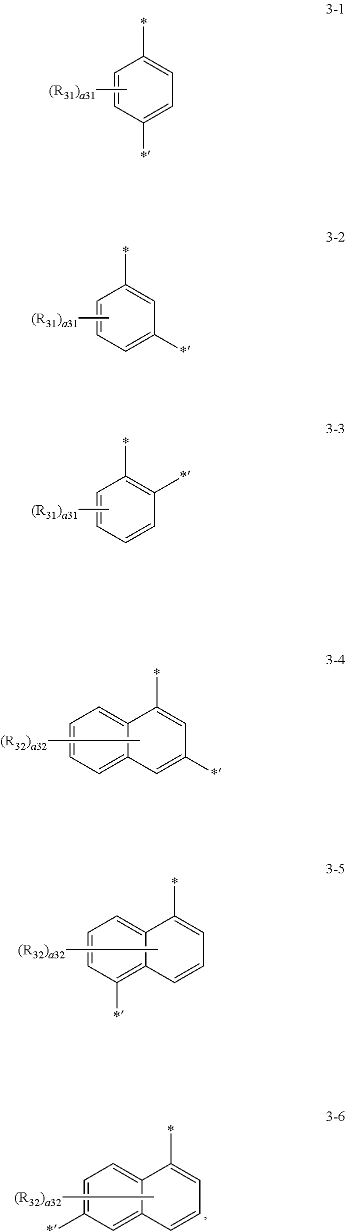
and
wherein, in Formulae 3-1 to 3-6,
R₃₁ and R₃₂ are each independently the same as defined with respect to R₁ in Formula 1,
a31 is an integer from 1 to 4, a32 is an integer from 1 to 6,
at least one of R₁ in the number of a1 is optionally linked to at least one of R₃₁ in the number of a31 or at least one of R₃₂ in the number of a32 to form a C₅-C₃₀ carbocyclic group unsubstituted or substituted with at least one R₁₀ₐ or a C₁-C₃₀ heterocyclic group unsubstituted or substituted with at least one R₁₀ₐ,
at least one of R₂ in the number of a2 is optionally linked to at least one of R₃₁ in the number of a31 or at least one of R₃₂ in the number of a32 to form a C₅-C₃₀ carbocyclic group unsubstituted or substituted with at least one R₁₀ₐ or a C₁-C₃₀ heterocyclic group unsubstituted or substituted with at least one R₁₀ₐ, and
* and *′ each indicate a binding site to a neighboring atom.

16. The amine-containing compound of claim 11, wherein the amine-containing compound satisfies any one selected from among Conditions i to iv:
Condition i)
Y₁ and Y₅ are each N, Y₂ is C(R₁₂), Yₛ is C(R₁₃), and Y₄ is C(R₁₄);
Condition ii)
Y₁ and Y₃ are each N, Y₂ is C(R₁₂), Y₄ is C(R₁₄), and Y₅ is C(R₁₅);
Condition iii)
Y₁ and Y₄ are each N, Y₂ is C(R₁₂), Yₛ is C(R₁₃), and Y₅ is C(R₁₅); and
Condition iv)
Y₁, Y₃, and Y₅ are each N, Y₂ is C(R₁₂), and Y₄ is C(R₁₄).

17. The amine-containing compound of claim 11, wherein Ar₂ is a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, or a naphthyridinyl group, each unsubstituted or substituted with at least one R₁₀ₐ.

18. The amine-containing compound of claim 11, wherein Ar₂ is a group represented by Formula 2.

19. The amine-containing compound of claim 11, wherein R₁ and R₂ are each independently:
deuterium; or
a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof.

20. The amine-containing compound of claim 11, wherein the amine-containing compound represented by Formula 1 is any one selected from among Compounds 1 to 694. 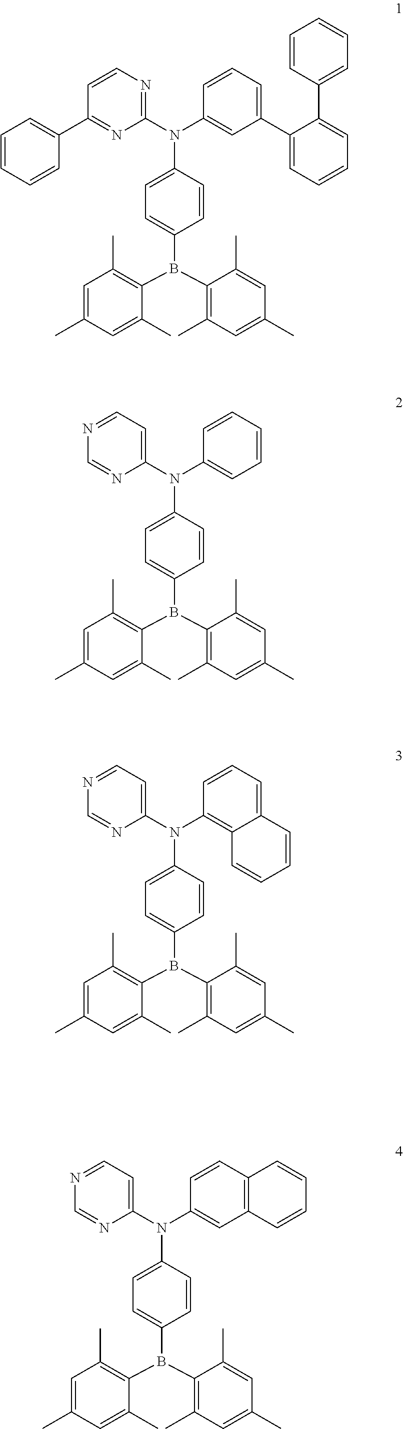 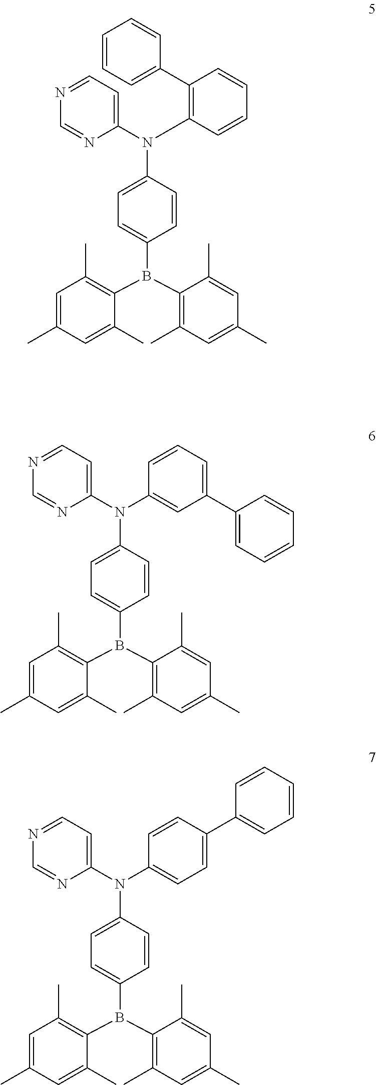 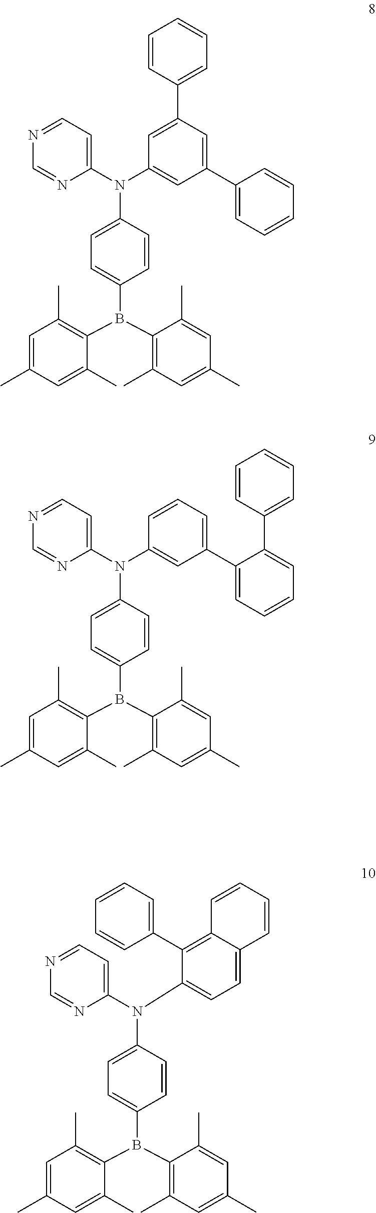 [SMILES: Cc1cc(C)c(B(c2ccc(N(c3cc(-c4ccccc4)cc(-c4ccccc4)c3)c3ccncn3)cc2)c2c(C)cc(C)cc2C)c(C)c1 ; Cc1cc(C)c(B(c2ccc(N(c3cccc(-c4ccccc4-c4ccccc4)c3)c3ccncn3)cc2)c2c(C)cc(C)cc2C)c(C)c1 ; Cc1cc(C)c(B(c2ccc(N(c3ccncn3)c3ccc4ccccc4c3-c3ccccc3)cc2)c2c(C)cc(C)cc2C)c(C)c1] 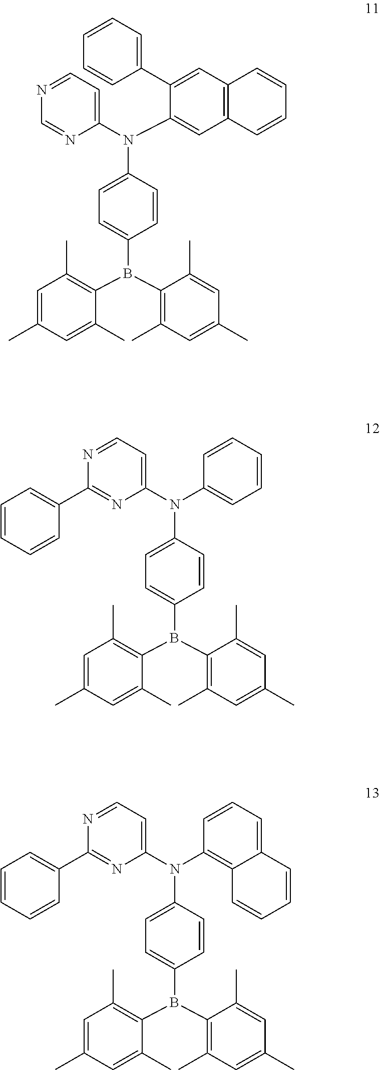 [SMILES: Cc1cc(C)c(B(c2ccc(N(c3ccncn3)c3cc4ccccc4cc3-c3ccccc3)cc2)c2c(C)cc(C)cc2C)c(C)c1 ; Cc1cc(C)c(B(c2ccc(N(c3ccccc3)c3ccnc(-c4ccccc4)n3)cc2)c2c(C)cc(C)cc2C)c(C)c1 ; Cc1cc(C)c(B(c2ccc(N(c3ccnc(-c4ccccc4)n3)c3cccc4ccccc34)cc2)c2c(C)cc(C)cc2C)c(C)c1] 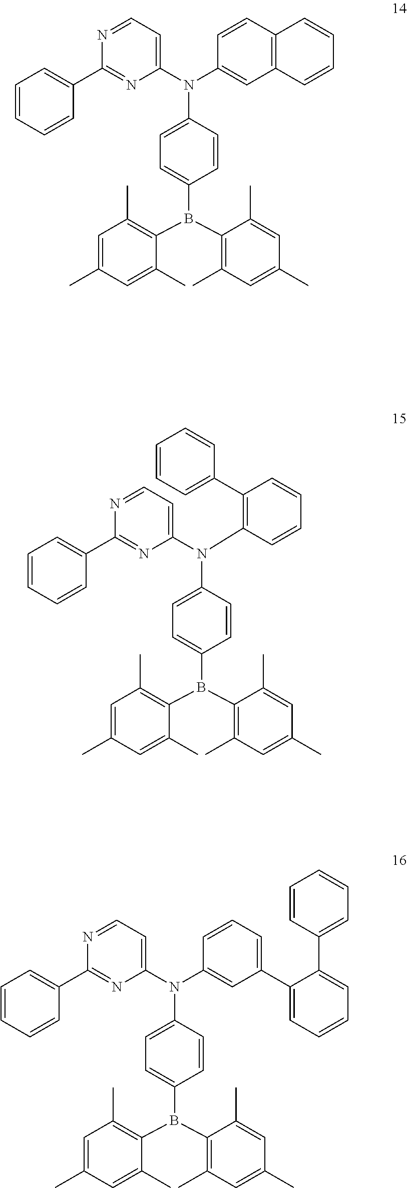 [SMILES: Cc1cc(C)c(B(c2ccc(N(c3ccc4ccccc4c3)c3ccnc(-c4ccccc4)n3)cc2)c2c(C)cc(C)cc2C)c(C)c1 ; Cc1cc(C)c(B(c2ccc(N(c3ccnc(-c4ccccc4)n3)c3ccccc3-c3ccccc3)cc2)c2c(C)cc(C)cc2C)c(C)c1 ; Cc1cc(C)c(B(c2ccc(N(c3cccc(-c4ccccc4-c4ccccc4)c3)c3ccnc(-c4ccccc4)n3)cc2)c2c(C)cc(C)cc2C)c(C)c1] 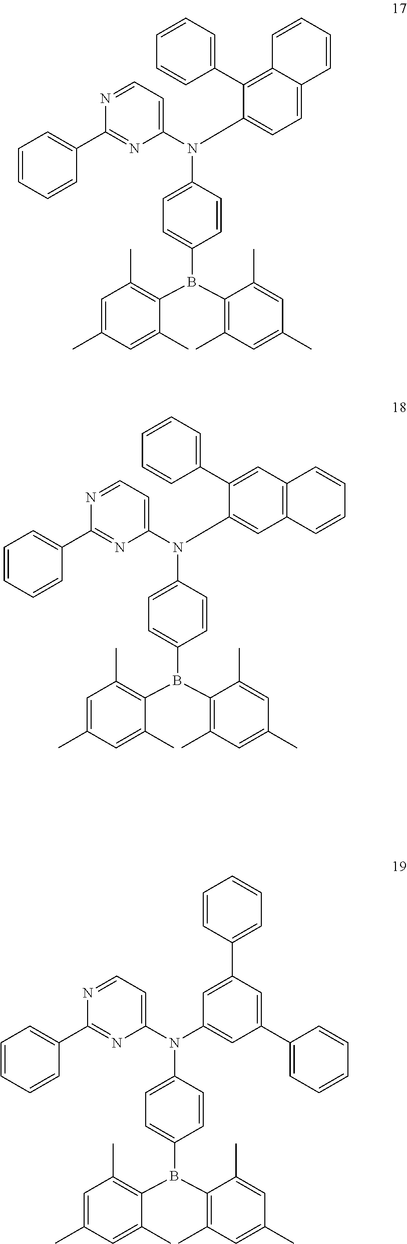 [SMILES: Cc1cc(C)c(B(c2ccc(N(c3ccnc(-c4ccccc4)n3)c3ccc4ccccc4c3-c3ccccc3)cc2)c2c(C)cc(C)cc2C)c(C)c1 ; Cc1cc(C)c(B(c2ccc(N(c3ccnc(-c4ccccc4)n3)c3cc4ccccc4cc3-c3ccccc3)cc2)c2c(C)cc(C)cc2C)c(C)c1 ; Cc1cc(C)c(B(c2ccc(N(c3cc(-c4ccccc4)cc(-c4ccccc4)c3)c3ccnc(-c4ccccc4)n3)cc2)c2c(C)cc(C)cc2C)c(C)c1] 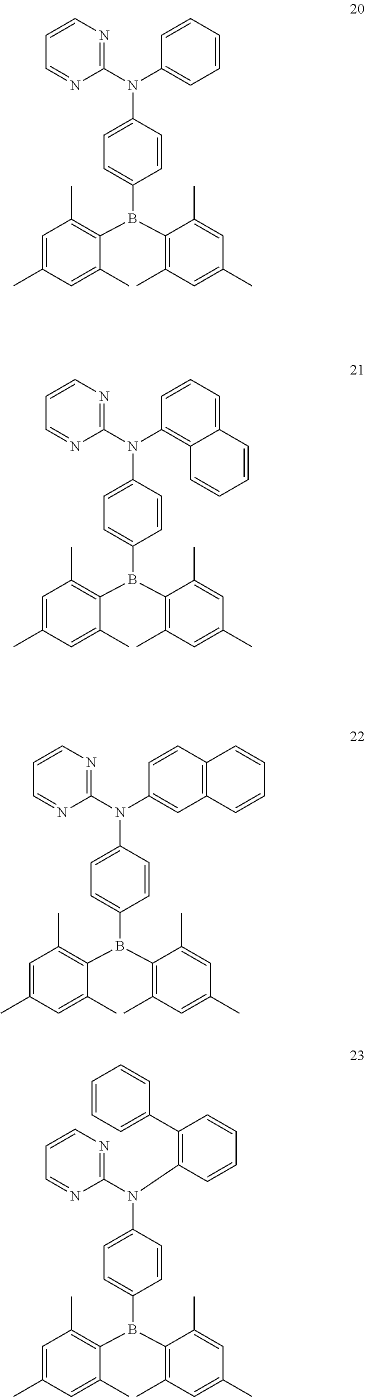 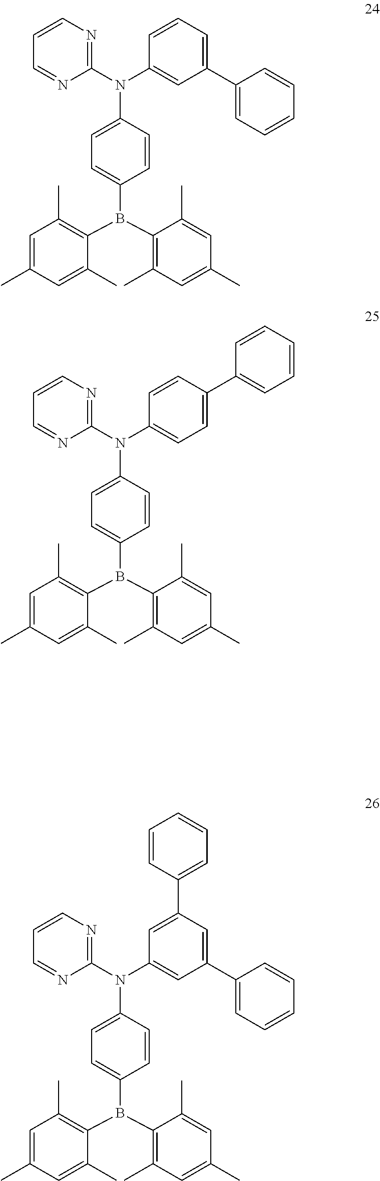 [SMILES: Cc1cc(C)c(B(c2ccc(N(c3cccc(-c4ccccc4)c3)c3ncccn3)cc2)c2c(C)cc(C)cc2C)c(C)c1 ; Cc1cc(C)c(B(c2ccc(N(c3ccc(-c4ccccc4)cc3)c3ncccn3)cc2)c2c(C)cc(C)cc2C)c(C)c1 ; Cc1cc(C)c(B(c2ccc(N(c3cc(-c4ccccc4)cc(-c4ccccc4)c3)c3ncccn3)cc2)c2c(C)cc(C)cc2C)c(C)c1] 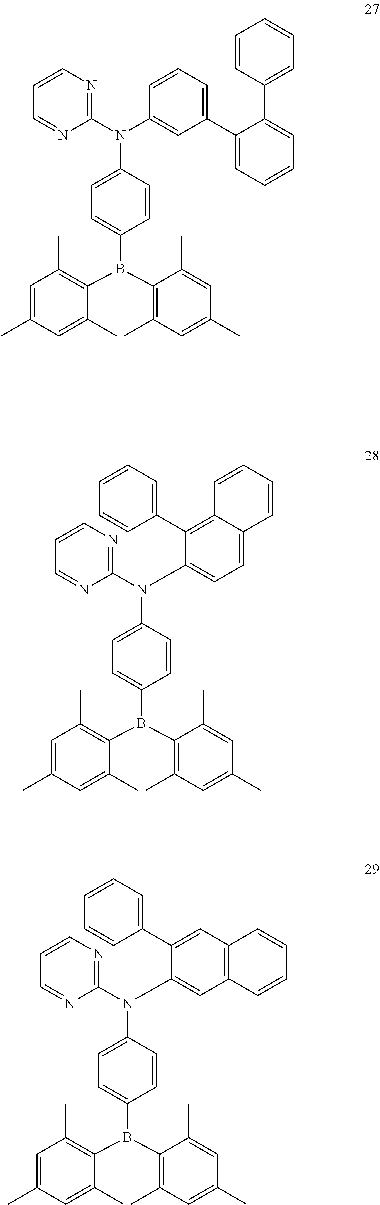 [SMILES: Cc1cc(C)c(B(c2ccc(N(c3cccc(-c4ccccc4-c4ccccc4)c3)c3ncccn3)cc2)c2c(C)cc(C)cc2C)c(C)c1 ; Cc1cc(C)c(B(c2ccc(N(c3ncccn3)c3ccc4ccccc4c3-c3ccccc3)cc2)c2c(C)cc(C)cc2C)c(C)c1 ; Cc1cc(C)c(B(c2ccc(N(c3ncccn3)c3cc4ccccc4cc3-c3ccccc3)cc2)c2c(C)cc(C)cc2C)c(C)c1] 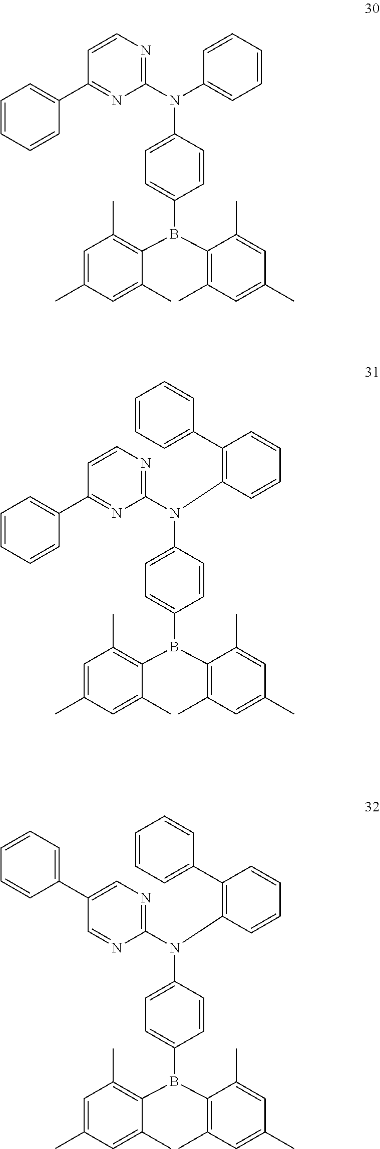 [SMILES: Cc1cc(C)c(B(c2ccc(N(c3ccccc3)c3nccc(-c4ccccc4)n3)cc2)c2c(C)cc(C)cc2C)c(C)c1 ; Cc1cc(C)c(B(c2ccc(N(c3nccc(-c4ccccc4)n3)c3ccccc3-c3ccccc3)cc2)c2c(C)cc(C)cc2C)c(C)c1 ; Cc1cc(C)c(B(c2ccc(N(c3ncc(-c4ccccc4)cn3)c3ccccc3-c3ccccc3)cc2)c2c(C)cc(C)cc2C)c(C)c1] 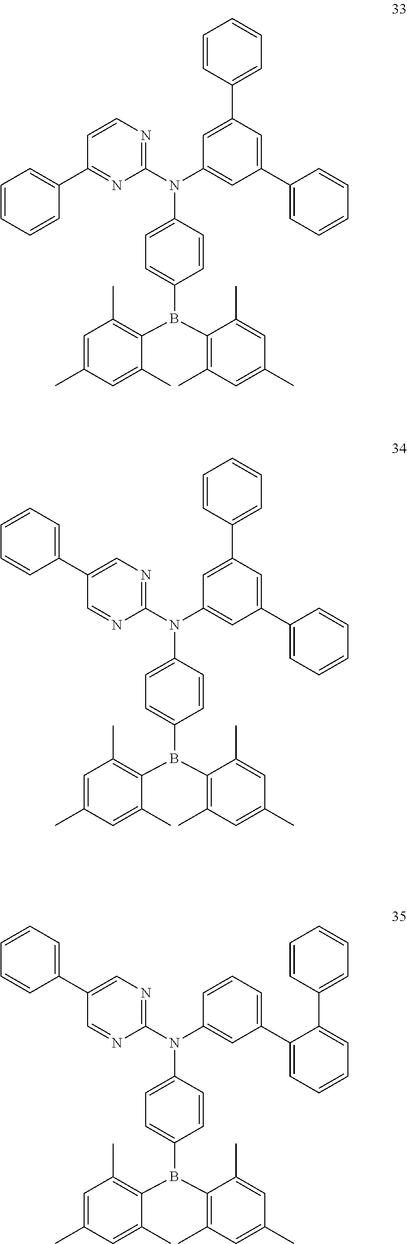 [SMILES: Cc1cc(C)c(B(c2ccc(N(c3cc(-c4ccccc4)cc(-c4ccccc4)c3)c3nccc(-c4ccccc4)n3)cc2)c2c(C)cc(C)cc2C)c(C)c1 ; Cc1cc(C)c(B(c2ccc(N(c3cc(-c4ccccc4)cc(-c4ccccc4)c3)c3ncc(-c4ccccc4)cn3)cc2)c2c(C)cc(C)cc2C)c(C)c1 ; Cc1cc(C)c(B(c2ccc(N(c3cccc(-c4ccccc4-c4ccccc4)c3)c3ncc(-c4ccccc4)cn3)cc2)c2c(C)cc(C)cc2C)c(C)c1] 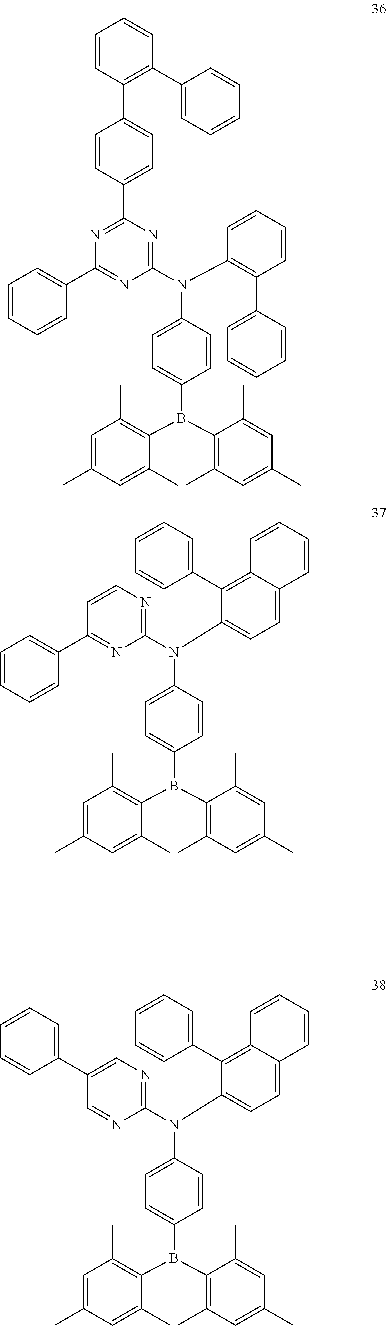 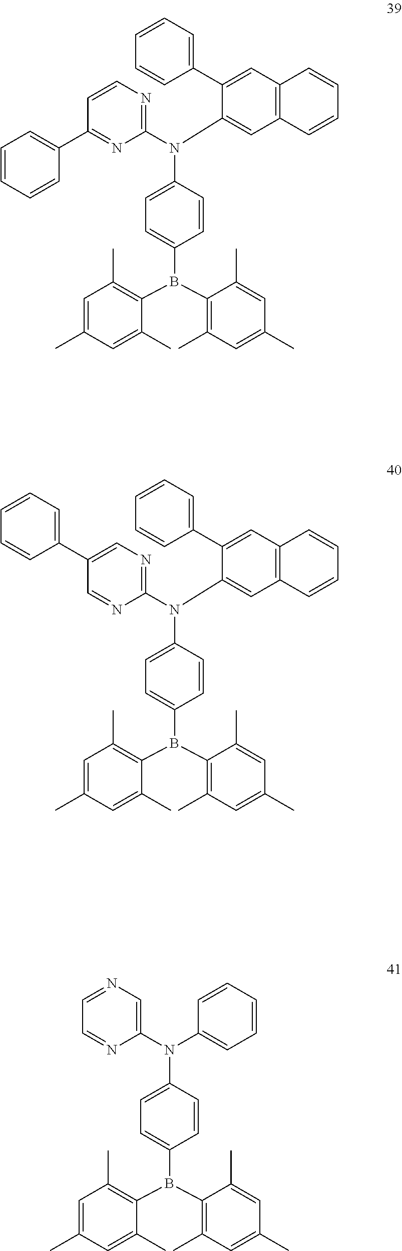 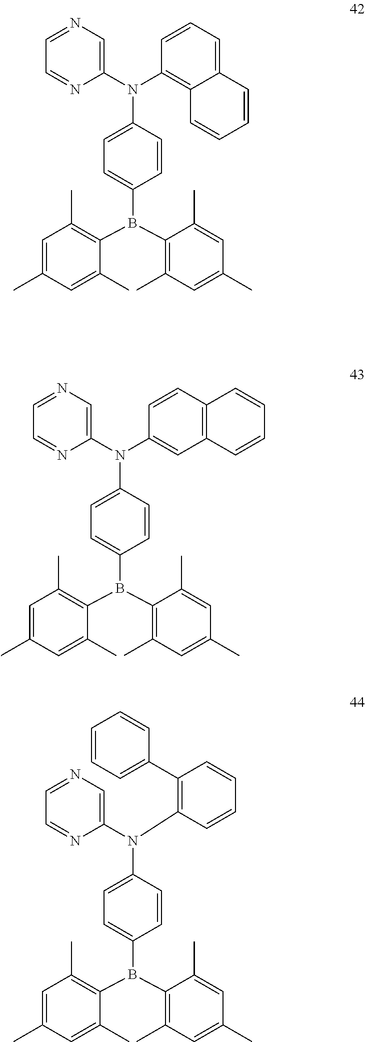 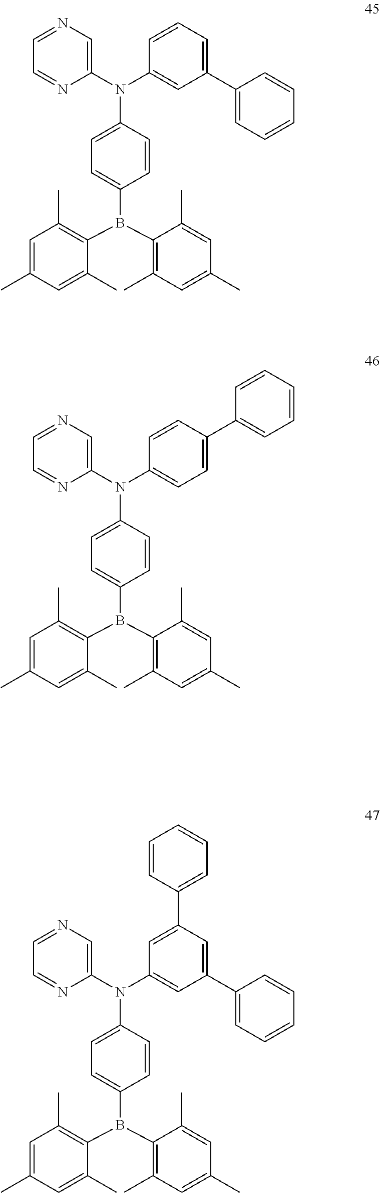 [SMILES: Cc1cc(C)c(B(c2ccc(N(c3cccc(-c4ccccc4)c3)c3cnccn3)cc2)c2c(C)cc(C)cc2C)c(C)c1 ; Cc1cc(C)c(B(c2ccc(N(c3ccc(-c4ccccc4)cc3)c3cnccn3)cc2)c2c(C)cc(C)cc2C)c(C)c1 ; Cc1cc(C)c(B(c2ccc(N(c3cc(-c4ccccc4)cc(-c4ccccc4)c3)c3cnccn3)cc2)c2c(C)cc(C)cc2C)c(C)c1] 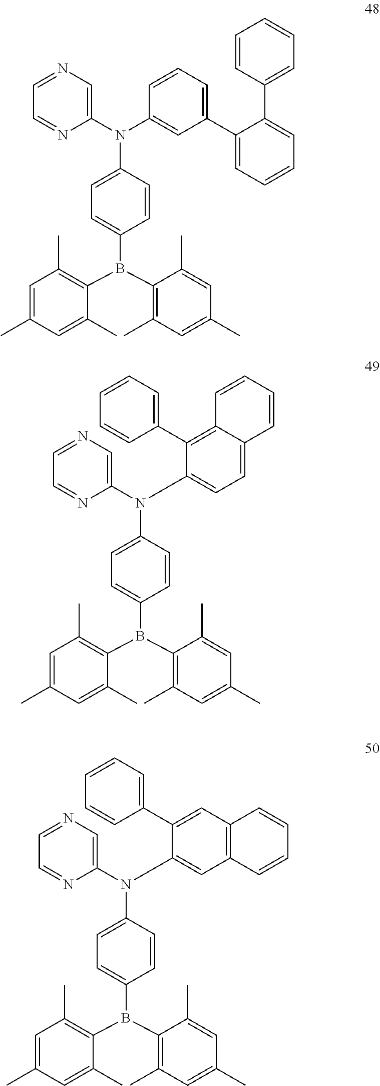 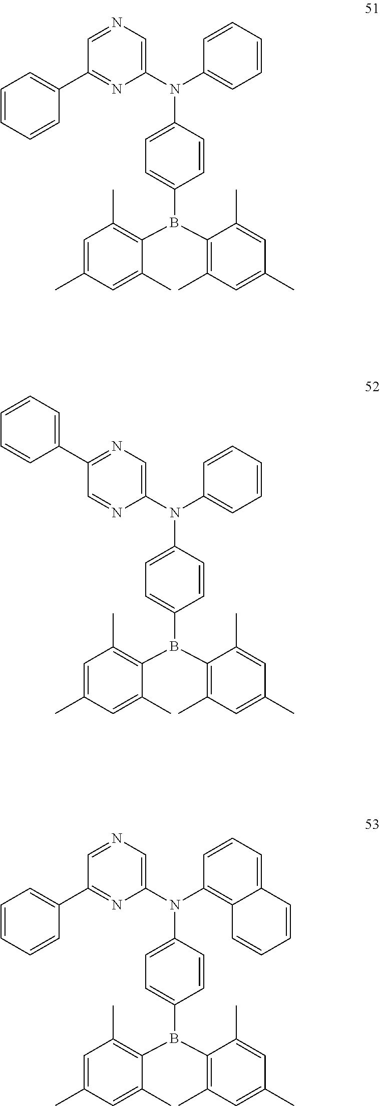 [SMILES: Cc1cc(C)c(B(c2ccc(N(c3ccccc3)c3cncc(-c4ccccc4)n3)cc2)c2c(C)cc(C)cc2C)c(C)c1 ; Cc1cc(C)c(B(c2ccc(N(c3ccccc3)c3cnc(-c4ccccc4)cn3)cc2)c2c(C)cc(C)cc2C)c(C)c1 ; Cc1cc(C)c(B(c2ccc(N(c3cncc(-c4ccccc4)n3)c3cccc4ccccc34)cc2)c2c(C)cc(C)cc2C)c(C)c1] 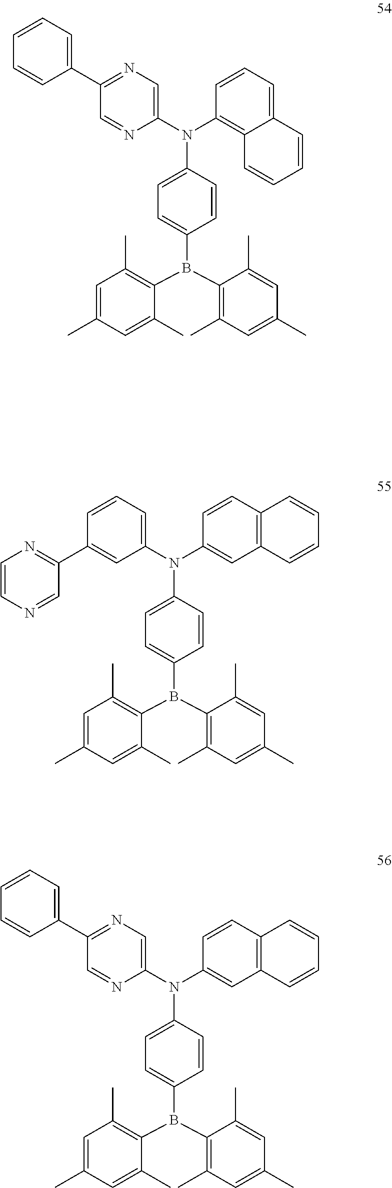 [SMILES: Cc1cc(C)c(B(c2ccc(N(c3cnc(-c4ccccc4)cn3)c3cccc4ccccc34)cc2)c2c(C)cc(C)cc2C)c(C)c1 ; Cc1cc(C)c(B(c2ccc(N(c3cccc(-c4cnccn4)c3)c3ccc4ccccc4c3)cc2)c2c(C)cc(C)cc2C)c(C)c1 ; Cc1cc(C)c(B(c2ccc(N(c3ccc4ccccc4c3)c3cnc(-c4ccccc4)cn3)cc2)c2c(C)cc(C)cc2C)c(C)c1] 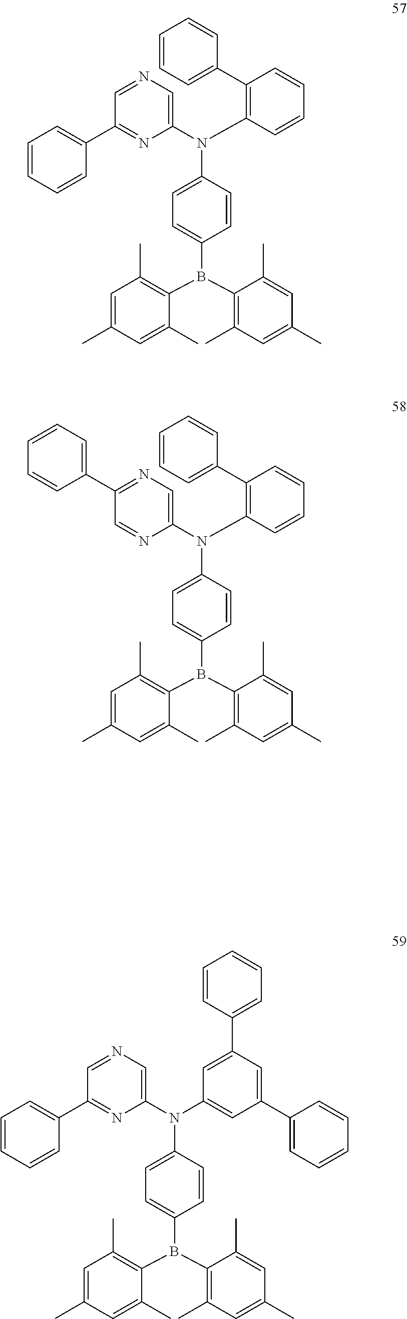 [SMILES: Cc1cc(C)c(B(c2ccc(N(c3cncc(-c4ccccc4)n3)c3ccccc3-c3ccccc3)cc2)c2c(C)cc(C)cc2C)c(C)c1 ; Cc1cc(C)c(B(c2ccc(N(c3cnc(-c4ccccc4)cn3)c3ccccc3-c3ccccc3)cc2)c2c(C)cc(C)cc2C)c(C)c1 ; Cc1cc(C)c(B(c2ccc(N(c3cc(-c4ccccc4)cc(-c4ccccc4)c3)c3cncc(-c4ccccc4)n3)cc2)c2c(C)cc(C)cc2C)c(C)c1] 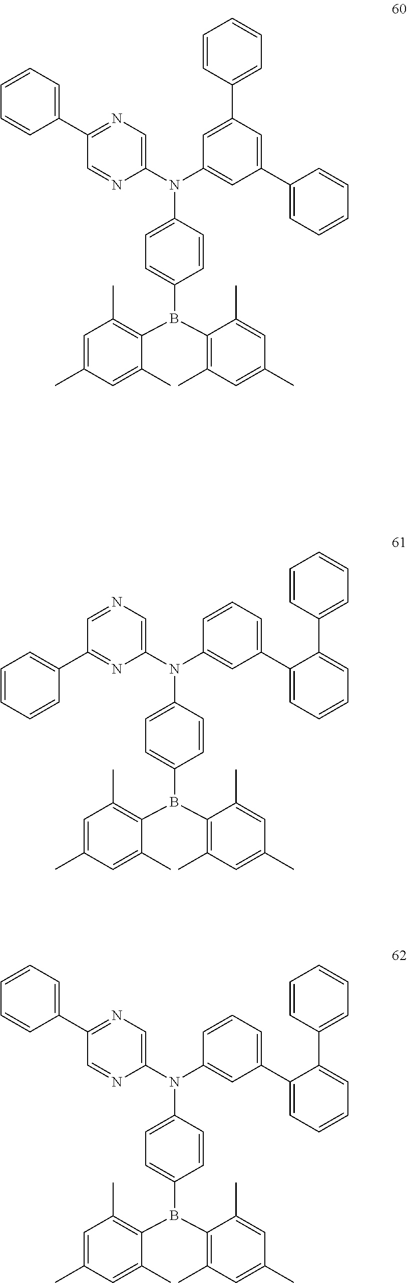 [SMILES: Cc1cc(C)c(B(c2ccc(N(c3cc(-c4ccccc4)cc(-c4ccccc4)c3)c3cnc(-c4ccccc4)cn3)cc2)c2c(C)cc(C)cc2C)c(C)c1 ; Cc1cc(C)c(B(c2ccc(N(c3cccc(-c4ccccc4-c4ccccc4)c3)c3cncc(-c4ccccc4)n3)cc2)c2c(C)cc(C)cc2C)c(C)c1 ; Cc1cc(C)c(B(c2ccc(N(c3cccc(-c4ccccc4-c4ccccc4)c3)c3cnc(-c4ccccc4)cn3)cc2)c2c(C)cc(C)cc2C)c(C)c1] 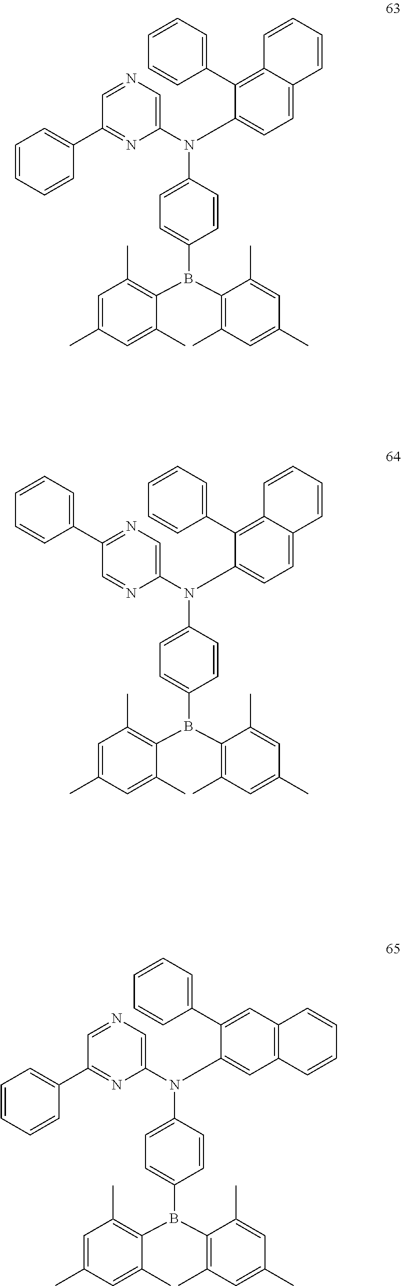 [SMILES: Cc1cc(C)c(B(c2ccc(N(c3cncc(-c4ccccc4)n3)c3ccc4ccccc4c3-c3ccccc3)cc2)c2c(C)cc(C)cc2C)c(C)c1 ; Cc1cc(C)c(B(c2ccc(N(c3cnc(-c4ccccc4)cn3)c3ccc4ccccc4c3-c3ccccc3)cc2)c2c(C)cc(C)cc2C)c(C)c1 ; Cc1cc(C)c(B(c2ccc(N(c3cncc(-c4ccccc4)n3)c3cc4ccccc4cc3-c3ccccc3)cc2)c2c(C)cc(C)cc2C)c(C)c1] 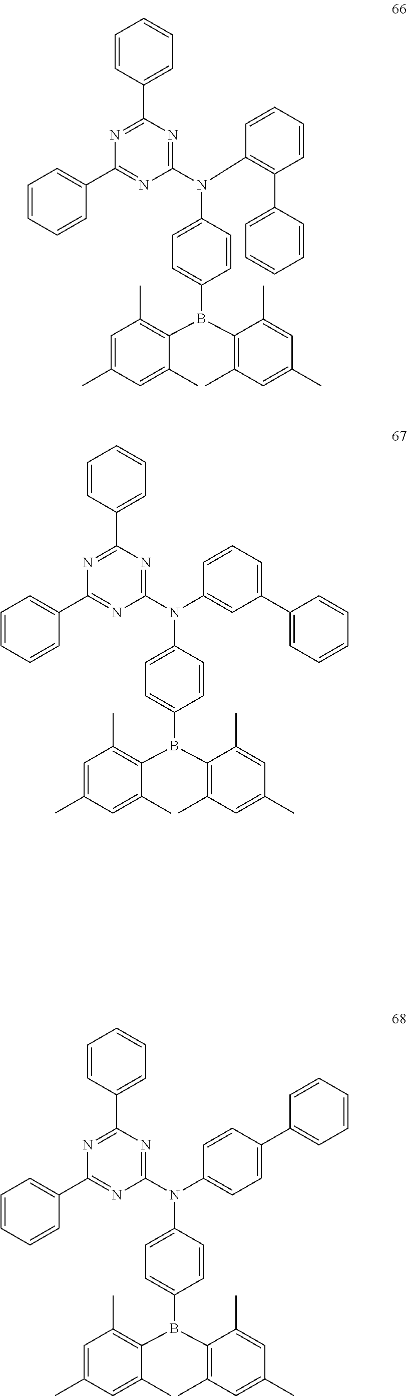 [SMILES: Cc1cc(C)c(B(c2ccc(N(c3nc(-c4ccccc4)nc(-c4ccccc4)n3)c3ccccc3-c3ccccc3)cc2)c2c(C)cc(C)cc2C)c(C)c1 ; Cc1cc(C)c(B(c2ccc(N(c3cccc(-c4ccccc4)c3)c3nc(-c4ccccc4)nc(-c4ccccc4)n3)cc2)c2c(C)cc(C)cc2C)c(C)c1 ; Cc1cc(C)c(B(c2ccc(N(c3ccc(-c4ccccc4)cc3)c3nc(-c4ccccc4)nc(-c4ccccc4)n3)cc2)c2c(C)cc(C)cc2C)c(C)c1] 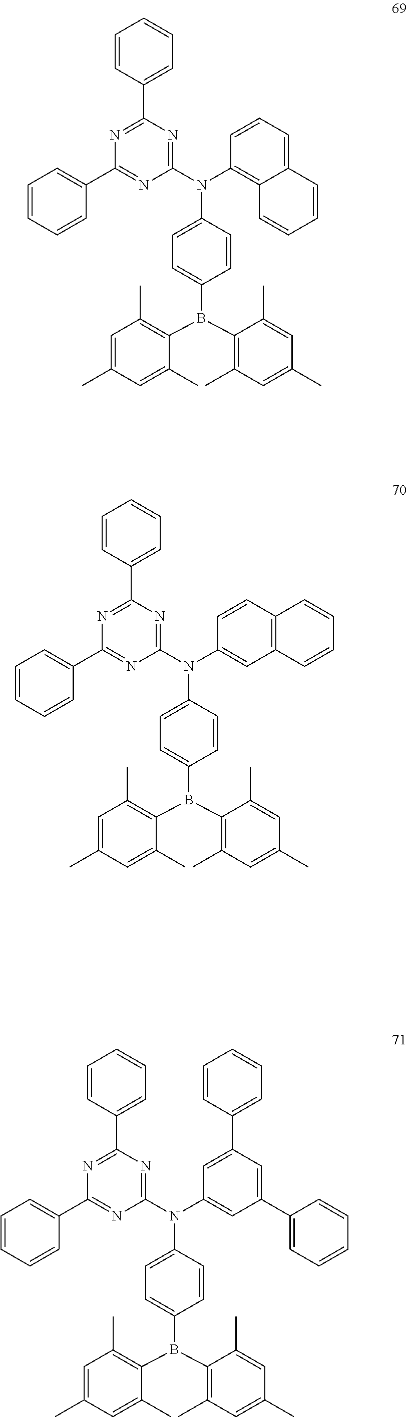 [SMILES: Cc1cc(C)c(B(c2ccc(N(c3nc(-c4ccccc4)nc(-c4ccccc4)n3)c3cccc4ccccc34)cc2)c2c(C)cc(C)cc2C)c(C)c1 ; Cc1cc(C)c(B(c2ccc(N(c3ccc4ccccc4c3)c3nc(-c4ccccc4)nc(-c4ccccc4)n3)cc2)c2c(C)cc(C)cc2C)c(C)c1 ; Cc1cc(C)c(B(c2ccc(N(c3cc(-c4ccccc4)cc(-c4ccccc4)c3)c3nc(-c4ccccc4)nc(-c4ccccc4)n3)cc2)c2c(C)cc(C)cc2C)c(C)c1] 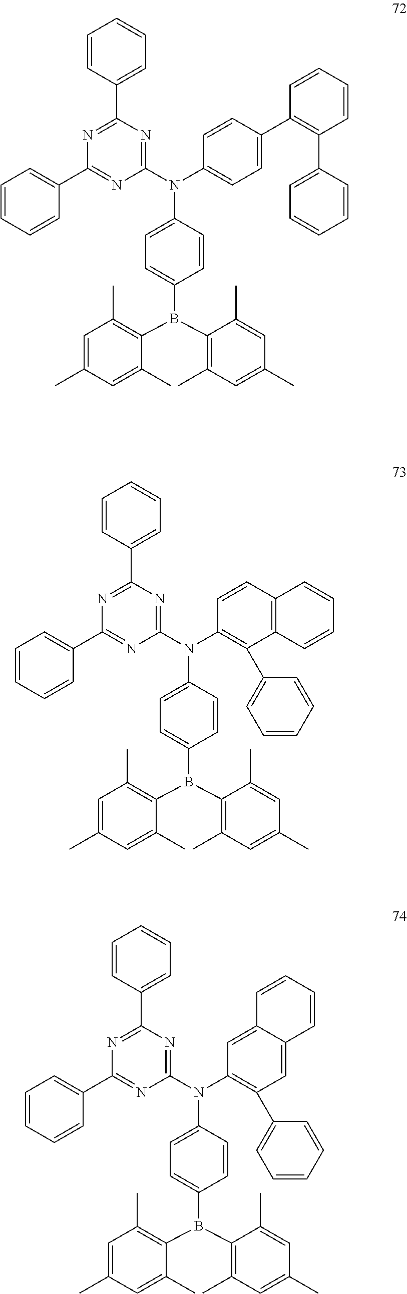 [SMILES: Cc1cc(C)c(B(c2ccc(N(c3ccc(-c4ccccc4-c4ccccc4)cc3)c3nc(-c4ccccc4)nc(-c4ccccc4)n3)cc2)c2c(C)cc(C)cc2C)c(C)c1 ; Cc1cc(C)c(B(c2ccc(N(c3nc(-c4ccccc4)nc(-c4ccccc4)n3)c3ccc4ccccc4c3-c3ccccc3)cc2)c2c(C)cc(C)cc2C)c(C)c1 ; Cc1cc(C)c(B(c2ccc(N(c3nc(-c4ccccc4)nc(-c4ccccc4)n3)c3cc4ccccc4cc3-c3ccccc3)cc2)c2c(C)cc(C)cc2C)c(C)c1] 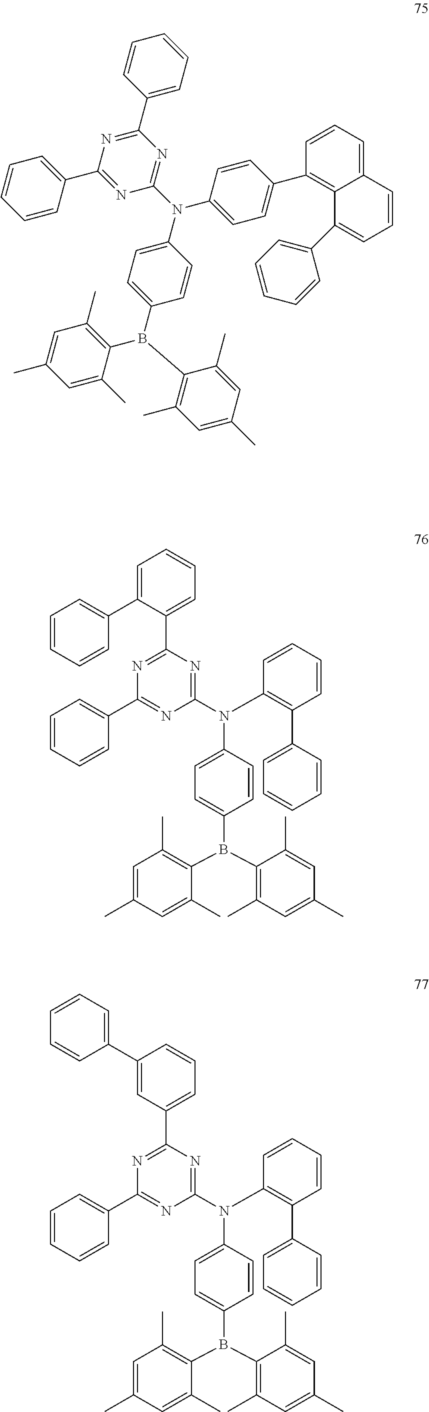 [SMILES: Cc1cc(C)c(B(c2ccc(N(c3ccc(-c4cccc5cccc(-c6ccccc6)c45)cc3)c3nc(-c4ccccc4)nc(-c4ccccc4)n3)cc2)c2c(C)cc(C)cc2C)c(C)c1 ; Cc1cc(C)c(B(c2ccc(N(c3nc(-c4ccccc4)nc(-c4ccccc4-c4ccccc4)n3)c3ccccc3-c3ccccc3)cc2)c2c(C)cc(C)cc2C)c(C)c1 ; Cc1cc(C)c(B(c2ccc(N(c3nc(-c4ccccc4)nc(-c4cccc(-c5ccccc5)c4)n3)c3ccccc3-c3ccccc3)cc2)c2c(C)cc(C)cc2C)c(C)c1] 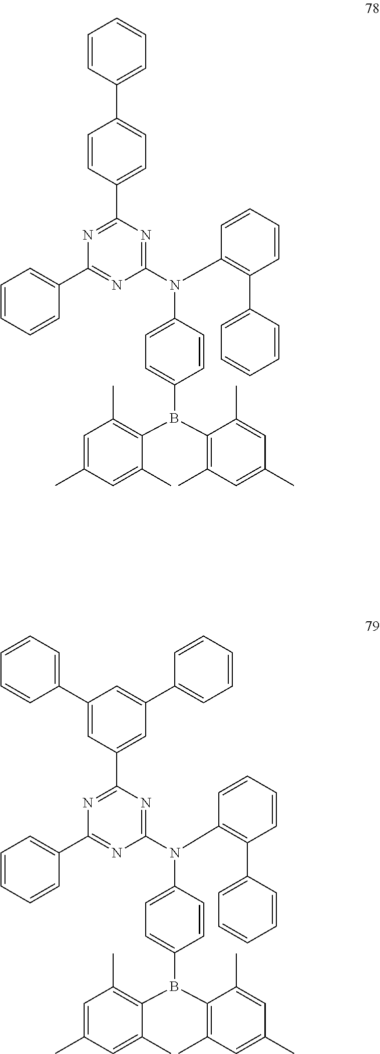 [SMILES: Cc1cc(C)c(B(c2ccc(N(c3nc(-c4ccccc4)nc(-c4ccc(-c5ccccc5)cc4)n3)c3ccccc3-c3ccccc3)cc2)c2c(C)cc(C)cc2C)c(C)c1 ; Cc1cc(C)c(B(c2ccc(N(c3nc(-c4ccccc4)nc(-c4cc(-c5ccccc5)cc(-c5ccccc5)c4)n3)c3ccccc3-c3ccccc3)cc2)c2c(C)cc(C)cc2C)c(C)c1] 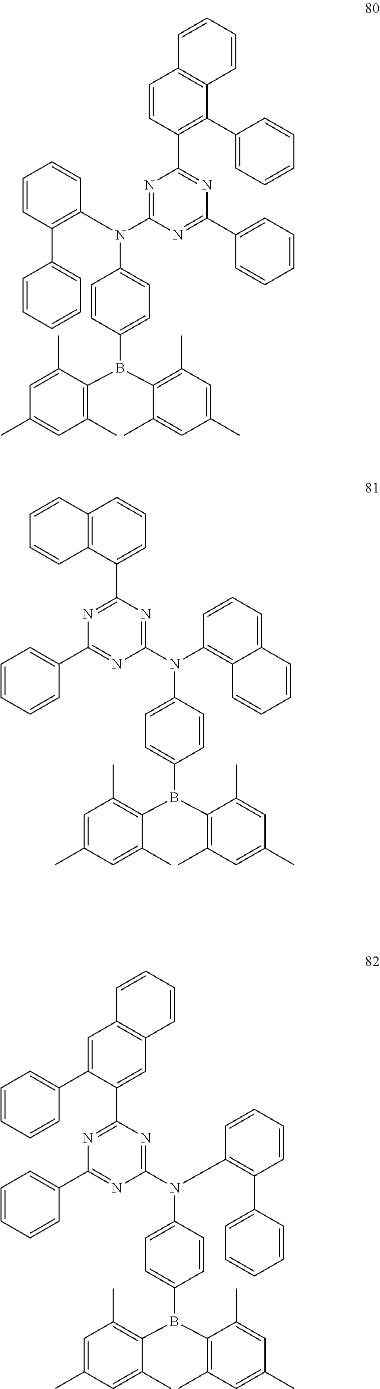 [SMILES: Cc1cc(C)c(B(c2ccc(N(c3nc(-c4ccccc4)nc(-c4ccc5ccccc5c4-c4ccccc4)n3)c3ccccc3-c3ccccc3)cc2)c2c(C)cc(C)cc2C)c(C)c1 ; Cc1cc(C)c(B(c2ccc(N(c3nc(-c4ccccc4)nc(-c4cccc5ccccc45)n3)c3cccc4ccccc34)cc2)c2c(C)cc(C)cc2C)c(C)c1 ; Cc1cc(C)c(B(c2ccc(N(c3nc(-c4ccccc4)nc(-c4cc5ccccc5cc4-c4ccccc4)n3)c3ccccc3-c3ccccc3)cc2)c2c(C)cc(C)cc2C)c(C)c1] 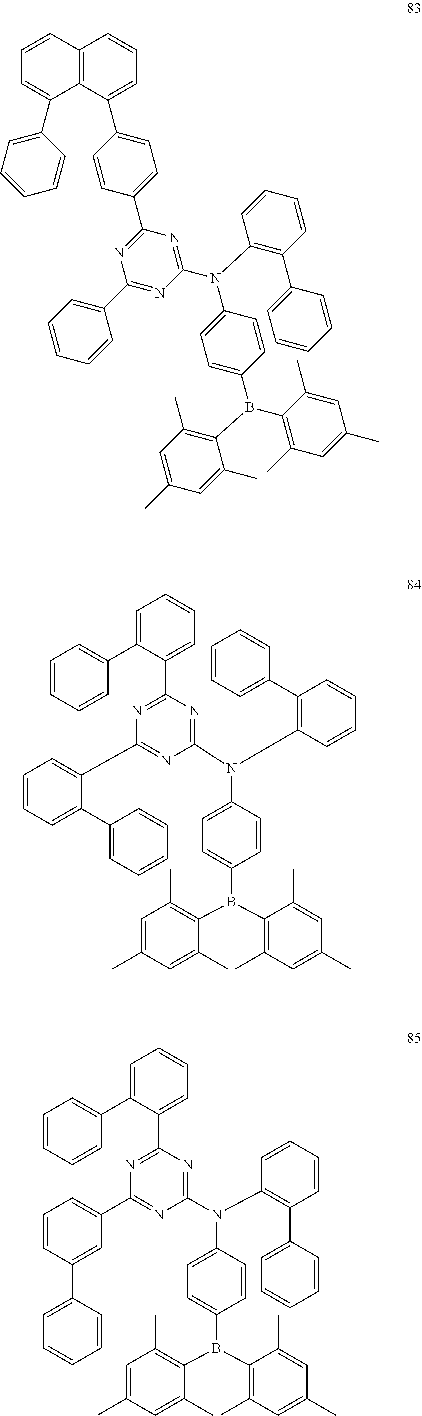 [SMILES: Cc1cc(C)c(B(c2ccc(N(c3nc(-c4ccccc4)nc(-c4ccc(-c5cccc6cccc(-c7ccccc7)c56)cc4)n3)c3ccccc3-c3ccccc3)cc2)c2c(C)cc(C)cc2C)c(C)c1 ; Cc1cc(C)c(B(c2ccc(N(c3nc(-c4ccccc4-c4ccccc4)nc(-c4ccccc4-c4ccccc4)n3)c3ccccc3-c3ccccc3)cc2)c2c(C)cc(C)cc2C)c(C)c1 ; Cc1cc(C)c(B(c2ccc(N(c3nc(-c4cccc(-c5ccccc5)c4)nc(-c4ccccc4-c4ccccc4)n3)c3ccccc3-c3ccccc3)cc2)c2c(C)cc(C)cc2C)c(C)c1] 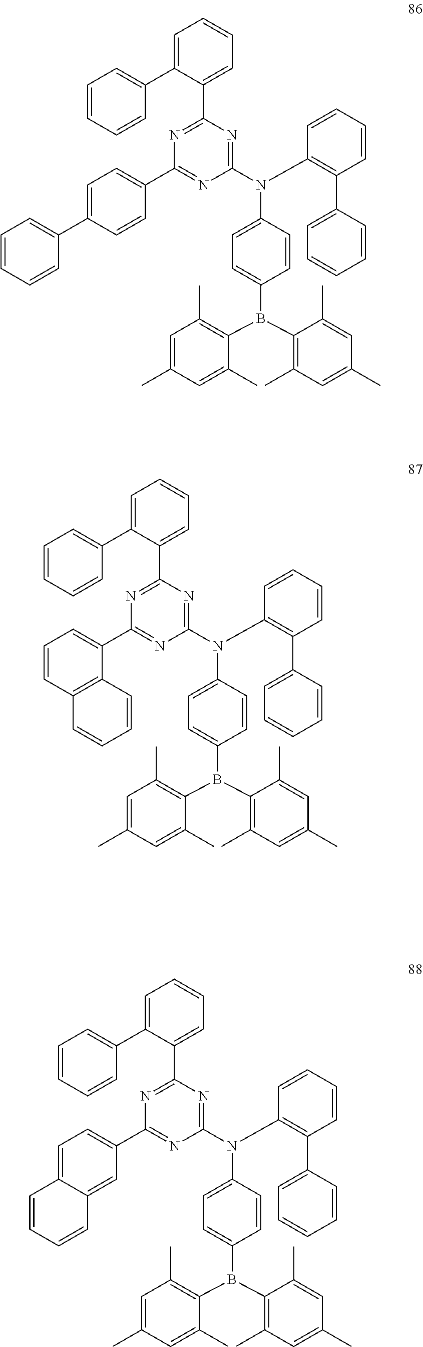 [SMILES: Cc1cc(C)c(B(c2ccc(N(c3nc(-c4ccc(-c5ccccc5)cc4)nc(-c4ccccc4-c4ccccc4)n3)c3ccccc3-c3ccccc3)cc2)c2c(C)cc(C)cc2C)c(C)c1 ; Cc1cc(C)c(B(c2ccc(N(c3nc(-c4ccccc4-c4ccccc4)nc(-c4cccc5ccccc45)n3)c3ccccc3-c3ccccc3)cc2)c2c(C)cc(C)cc2C)c(C)c1 ; Cc1cc(C)c(B(c2ccc(N(c3nc(-c4ccc5ccccc5c4)nc(-c4ccccc4-c4ccccc4)n3)c3ccccc3-c3ccccc3)cc2)c2c(C)cc(C)cc2C)c(C)c1] 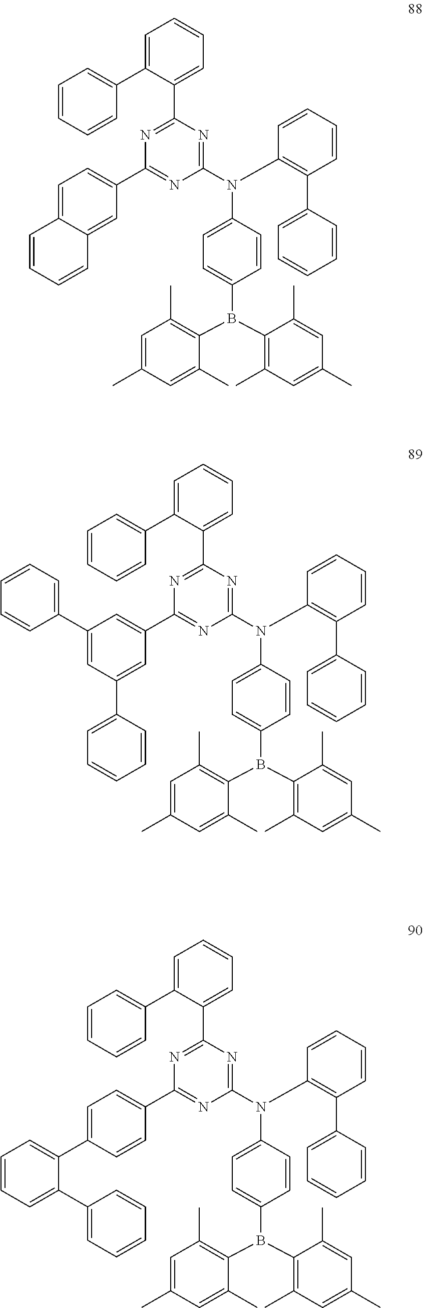 [SMILES: Cc1cc(C)c(B(c2ccc(N(c3nc(-c4ccc5ccccc5c4)nc(-c4ccccc4-c4ccccc4)n3)c3ccccc3-c3ccccc3)cc2)c2c(C)cc(C)cc2C)c(C)c1 ; Cc1cc(C)c(B(c2ccc(N(c3nc(-c4cc(-c5ccccc5)cc(-c5ccccc5)c4)nc(-c4ccccc4-c4ccccc4)n3)c3ccccc3-c3ccccc3)cc2)c2c(C)cc(C)cc2C)c(C)c1 ; Cc1cc(C)c(B(c2ccc(N(c3nc(-c4ccc(-c5ccccc5-c5ccccc5)cc4)nc(-c4ccccc4-c4ccccc4)n3)c3ccccc3-c3ccccc3)cc2)c2c(C)cc(C)cc2C)c(C)c1] 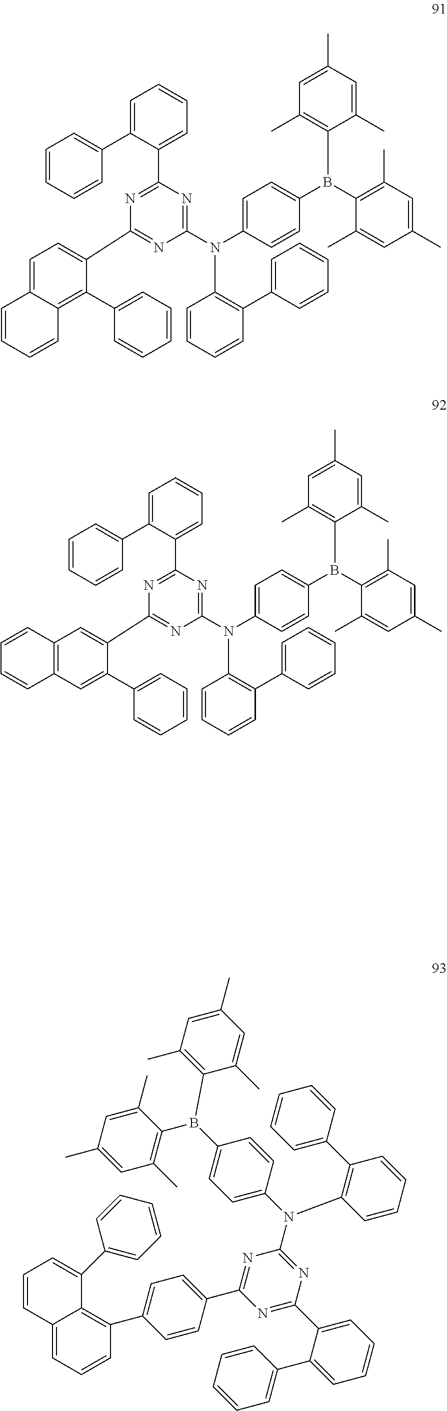 [SMILES: Cc1cc(C)c(B(c2ccc(N(c3nc(-c4ccccc4-c4ccccc4)nc(-c4ccc5ccccc5c4-c4ccccc4)n3)c3ccccc3-c3ccccc3)cc2)c2c(C)cc(C)cc2C)c(C)c1 ; Cc1cc(C)c(B(c2ccc(N(c3nc(-c4ccccc4-c4ccccc4)nc(-c4cc5ccccc5cc4-c4ccccc4)n3)c3ccccc3-c3ccccc3)cc2)c2c(C)cc(C)cc2C)c(C)c1 ; Cc1cc(C)c(B(c2ccc(N(c3nc(-c4ccc(-c5cccc6cccc(-c7ccccc7)c56)cc4)nc(-c4ccccc4-c4ccccc4)n3)c3ccccc3-c3ccccc3)cc2)c2c(C)cc(C)cc2C)c(C)c1] 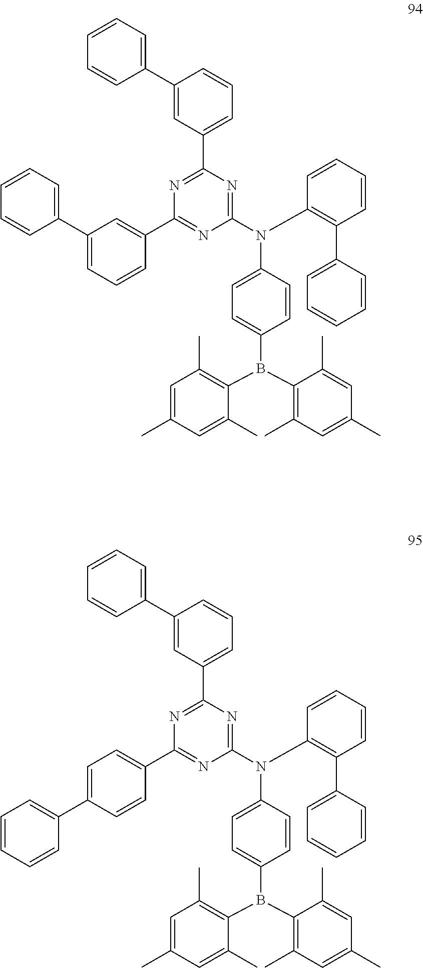 [SMILES: Cc1cc(C)c(B(c2ccc(N(c3nc(-c4cccc(-c5ccccc5)c4)nc(-c4cccc(-c5ccccc5)c4)n3)c3ccccc3-c3ccccc3)cc2)c2c(C)cc(C)cc2C)c(C)c1 ; Cc1cc(C)c(B(c2ccc(N(c3nc(-c4ccc(-c5ccccc5)cc4)nc(-c4cccc(-c5ccccc5)c4)n3)c3ccccc3-c3ccccc3)cc2)c2c(C)cc(C)cc2C)c(C)c1] 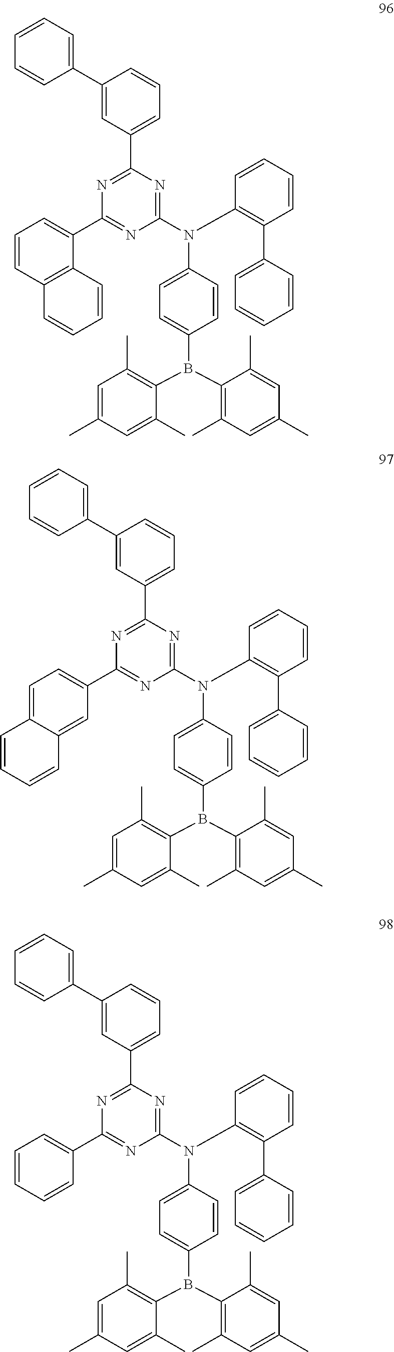 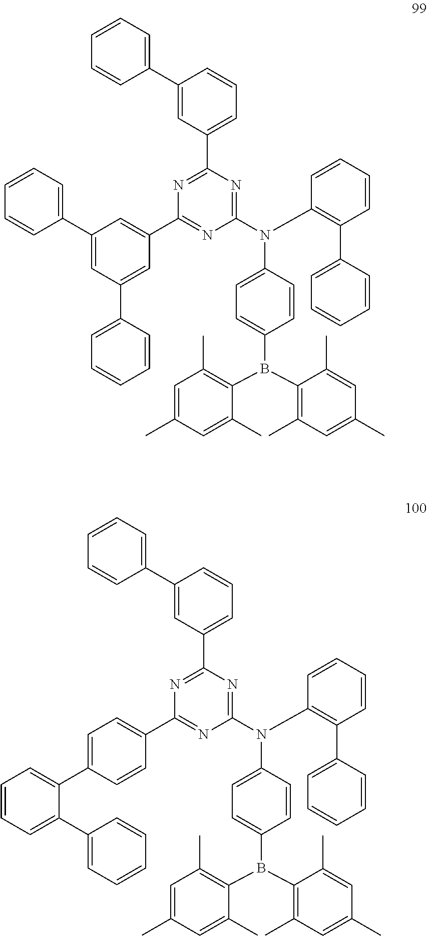 [SMILES: Cc1cc(C)c(B(c2ccc(N(c3nc(-c4cccc(-c5ccccc5)c4)nc(-c4cc(-c5ccccc5)cc(-c5ccccc5)c4)n3)c3ccccc3-c3ccccc3)cc2)c2c(C)cc(C)cc2C)c(C)c1 ; Cc1cc(C)c(B(c2ccc(N(c3nc(-c4ccc(-c5ccccc5-c5ccccc5)cc4)nc(-c4cccc(-c5ccccc5)c4)n3)c3ccccc3-c3ccccc3)cc2)c2c(C)cc(C)cc2C)c(C)c1] 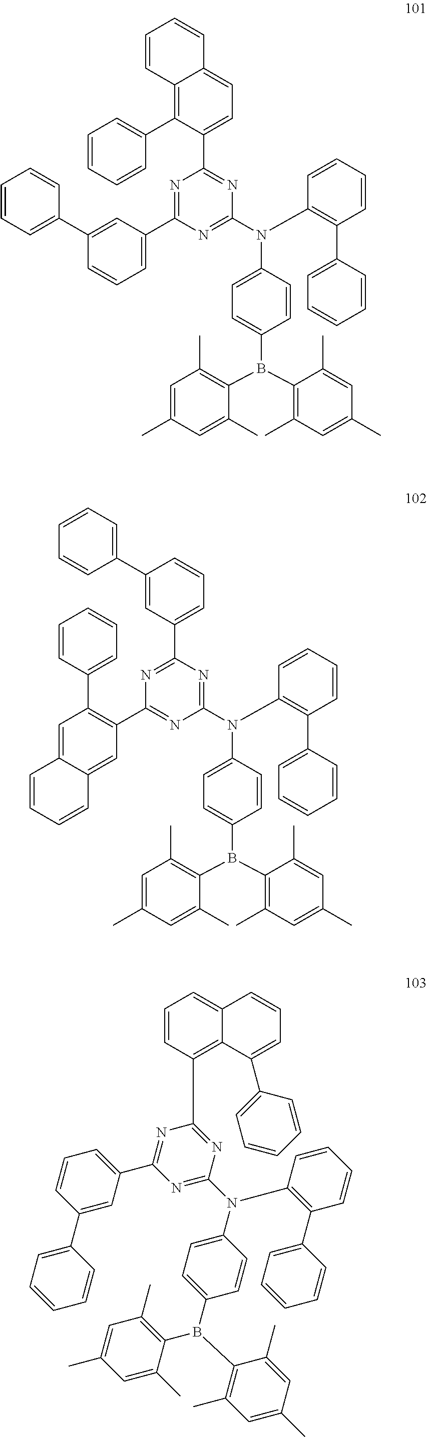 [SMILES: Cc1cc(C)c(B(c2ccc(N(c3nc(-c4cccc(-c5ccccc5)c4)nc(-c4ccc5ccccc5c4-c4ccccc4)n3)c3ccccc3-c3ccccc3)cc2)c2c(C)cc(C)cc2C)c(C)c1 ; Cc1cc(C)c(B(c2ccc(N(c3nc(-c4cccc(-c5ccccc5)c4)nc(-c4cc5ccccc5cc4-c4ccccc4)n3)c3ccccc3-c3ccccc3)cc2)c2c(C)cc(C)cc2C)c(C)c1 ; Cc1cc(C)c(B(c2ccc(N(c3nc(-c4cccc(-c5ccccc5)c4)nc(-c4cccc5cccc(-c6ccccc6)c45)n3)c3ccccc3-c3ccccc3)cc2)c2c(C)cc(C)cc2C)c(C)c1] 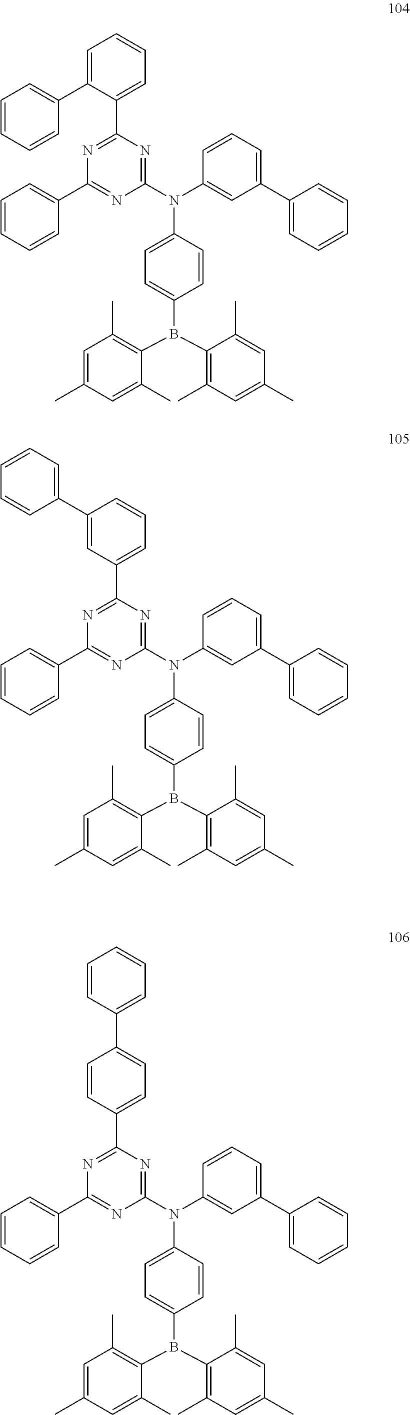 [SMILES: Cc1cc(C)c(B(c2ccc(N(c3cccc(-c4ccccc4)c3)c3nc(-c4ccccc4)nc(-c4ccccc4-c4ccccc4)n3)cc2)c2c(C)cc(C)cc2C)c(C)c1 ; Cc1cc(C)c(B(c2ccc(N(c3cccc(-c4ccccc4)c3)c3nc(-c4ccccc4)nc(-c4cccc(-c5ccccc5)c4)n3)cc2)c2c(C)cc(C)cc2C)c(C)c1 ; Cc1cc(C)c(B(c2ccc(N(c3cccc(-c4ccccc4)c3)c3nc(-c4ccccc4)nc(-c4ccc(-c5ccccc5)cc4)n3)cc2)c2c(C)cc(C)cc2C)c(C)c1] 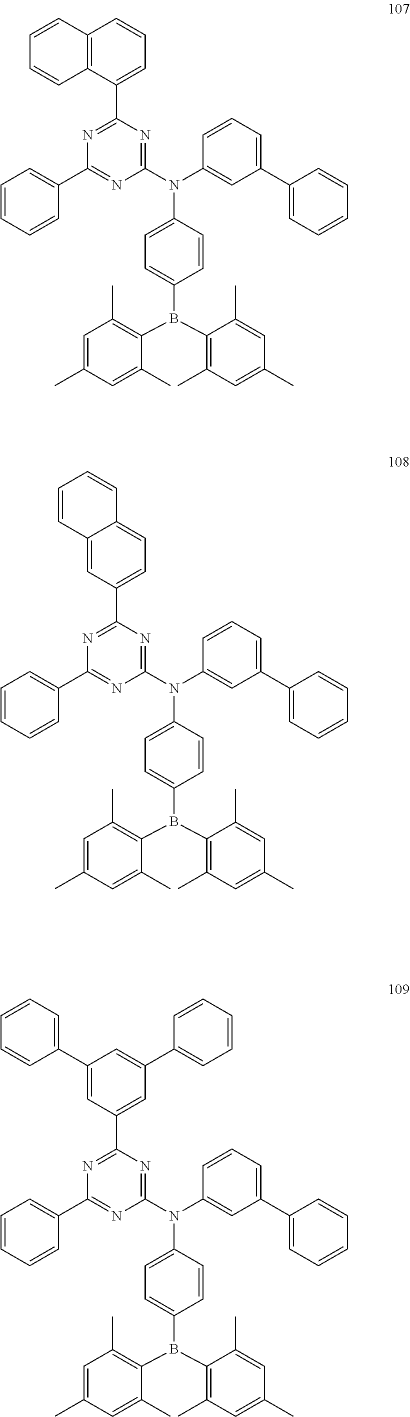 [SMILES: Cc1cc(C)c(B(c2ccc(N(c3cccc(-c4ccccc4)c3)c3nc(-c4ccccc4)nc(-c4cccc5ccccc45)n3)cc2)c2c(C)cc(C)cc2C)c(C)c1 ; Cc1cc(C)c(B(c2ccc(N(c3cccc(-c4ccccc4)c3)c3nc(-c4ccccc4)nc(-c4ccc5ccccc5c4)n3)cc2)c2c(C)cc(C)cc2C)c(C)c1 ; Cc1cc(C)c(B(c2ccc(N(c3cccc(-c4ccccc4)c3)c3nc(-c4ccccc4)nc(-c4cc(-c5ccccc5)cc(-c5ccccc5)c4)n3)cc2)c2c(C)cc(C)cc2C)c(C)c1] 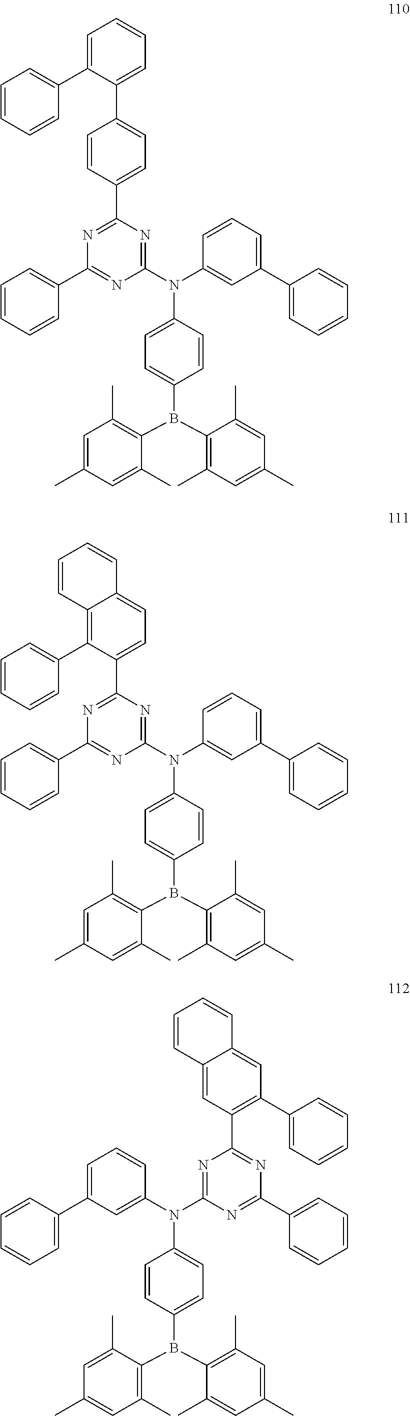 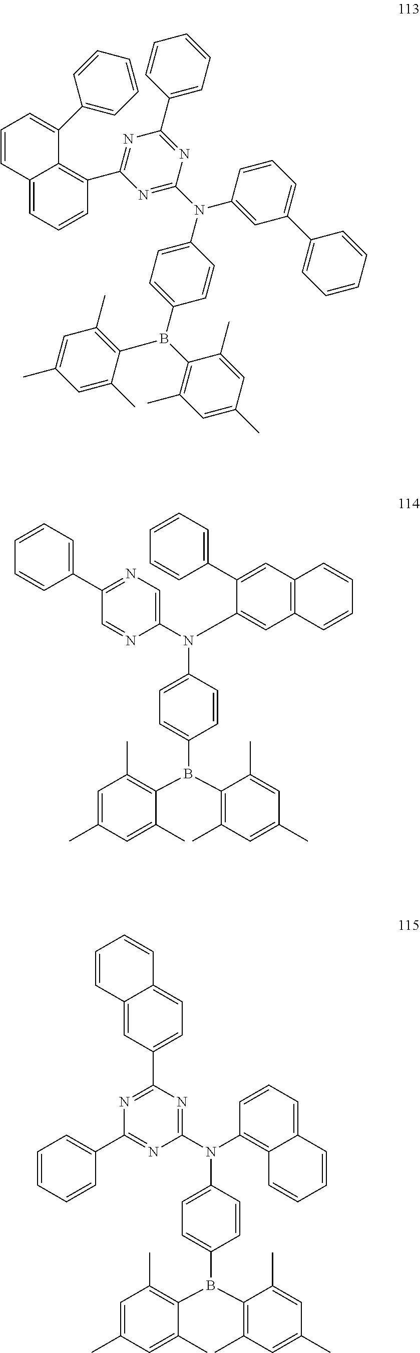 [SMILES: Cc1cc(C)c(B(c2ccc(N(c3cccc(-c4ccccc4)c3)c3nc(-c4ccccc4)nc(-c4cccc5cccc(-c6ccccc6)c45)n3)cc2)c2c(C)cc(C)cc2C)c(C)c1 ; Cc1cc(C)c(B(c2ccc(N(c3cnc(-c4ccccc4)cn3)c3cc4ccccc4cc3-c3ccccc3)cc2)c2c(C)cc(C)cc2C)c(C)c1 ; Cc1cc(C)c(B(c2ccc(N(c3nc(-c4ccccc4)nc(-c4ccc5ccccc5c4)n3)c3cccc4ccccc34)cc2)c2c(C)cc(C)cc2C)c(C)c1] 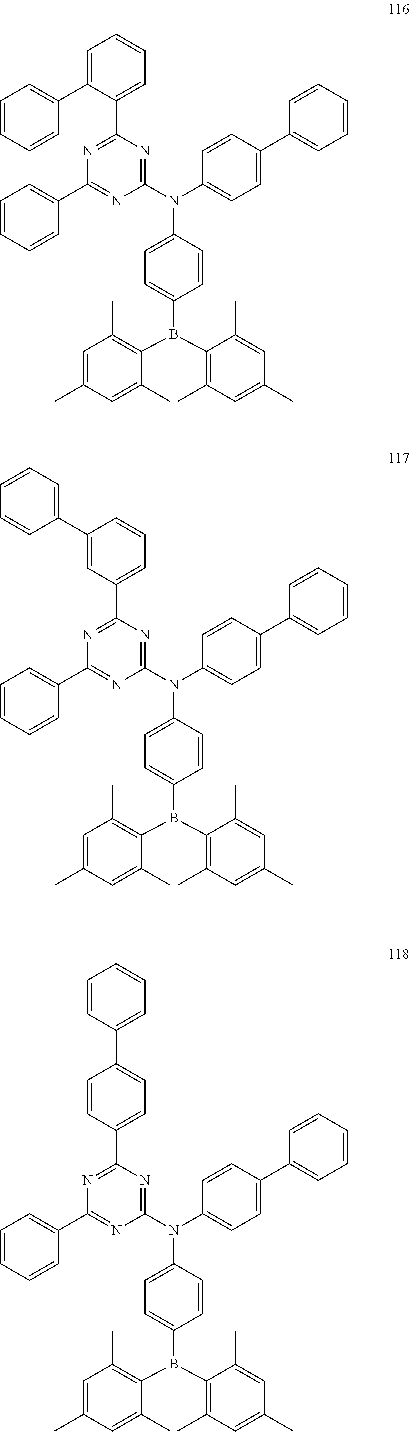 [SMILES: Cc1cc(C)c(B(c2ccc(N(c3ccc(-c4ccccc4)cc3)c3nc(-c4ccccc4)nc(-c4ccccc4-c4ccccc4)n3)cc2)c2c(C)cc(C)cc2C)c(C)c1 ; Cc1cc(C)c(B(c2ccc(N(c3ccc(-c4ccccc4)cc3)c3nc(-c4ccccc4)nc(-c4cccc(-c5ccccc5)c4)n3)cc2)c2c(C)cc(C)cc2C)c(C)c1 ; Cc1cc(C)c(B(c2ccc(N(c3ccc(-c4ccccc4)cc3)c3nc(-c4ccccc4)nc(-c4ccc(-c5ccccc5)cc4)n3)cc2)c2c(C)cc(C)cc2C)c(C)c1] 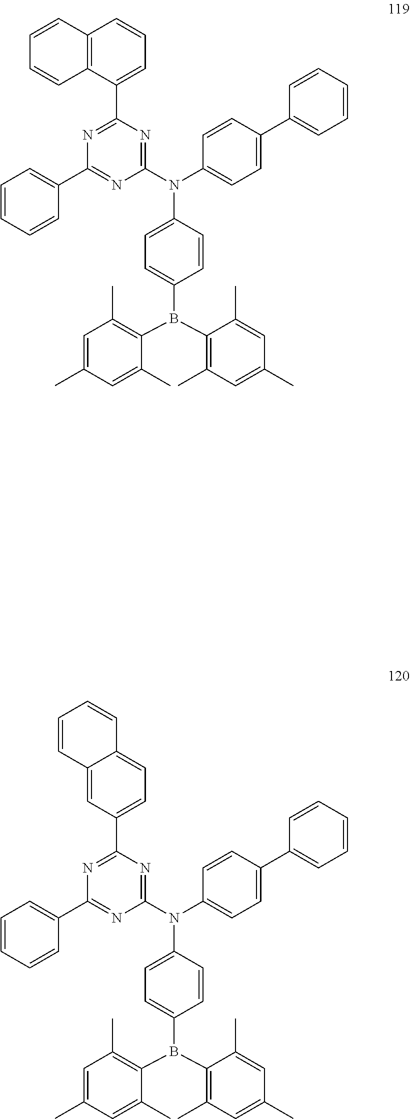 [SMILES: Cc1cc(C)c(B(c2ccc(N(c3ccc(-c4ccccc4)cc3)c3nc(-c4ccccc4)nc(-c4cccc5ccccc45)n3)cc2)c2c(C)cc(C)cc2C)c(C)c1 ; Cc1cc(C)c(B(c2ccc(N(c3ccc(-c4ccccc4)cc3)c3nc(-c4ccccc4)nc(-c4ccc5ccccc5c4)n3)cc2)c2c(C)cc(C)cc2C)c(C)c1] 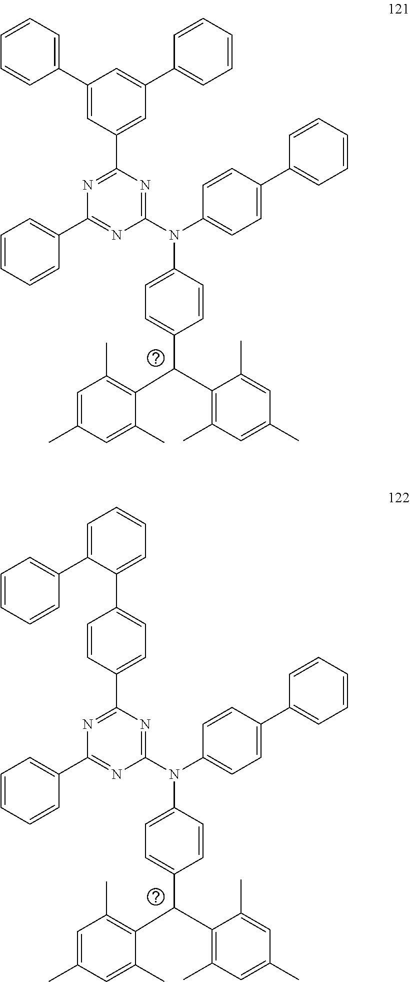 [SMILES: Cc1cc(C)c(C(c2ccc(N(c3ccc(-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc(-c4ccccc4)cc3)c3nc(-c4ccccc4)nc(-c4ccc(-c5ccccc5-c5ccccc5)cc4)n3)cc2)c2c(C)cc(C)cc2C)c(C)c1] 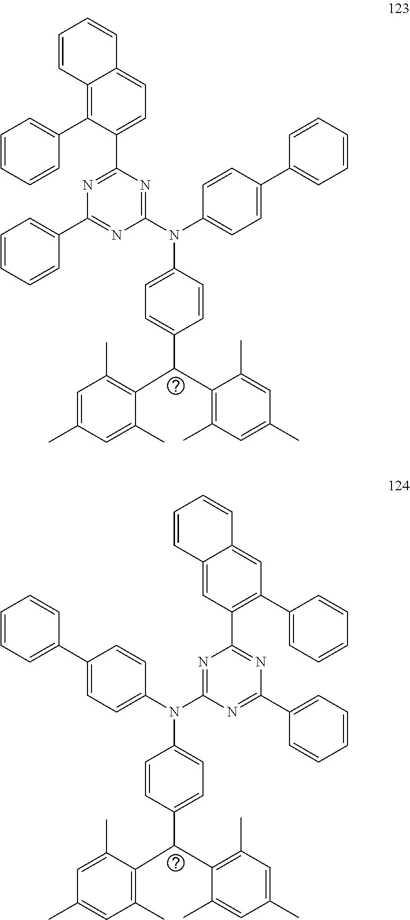 [SMILES: Cc1cc(C)c(C(c2ccc(N(c3ccc(-c4ccccc4)cc3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc(-c4ccccc4)cc3)c3nc(-c4ccccc4)nc(-c4cc5ccccc5cc4-c4ccccc4)n3)cc2)c2c(C)cc(C)cc2C)c(C)c1] 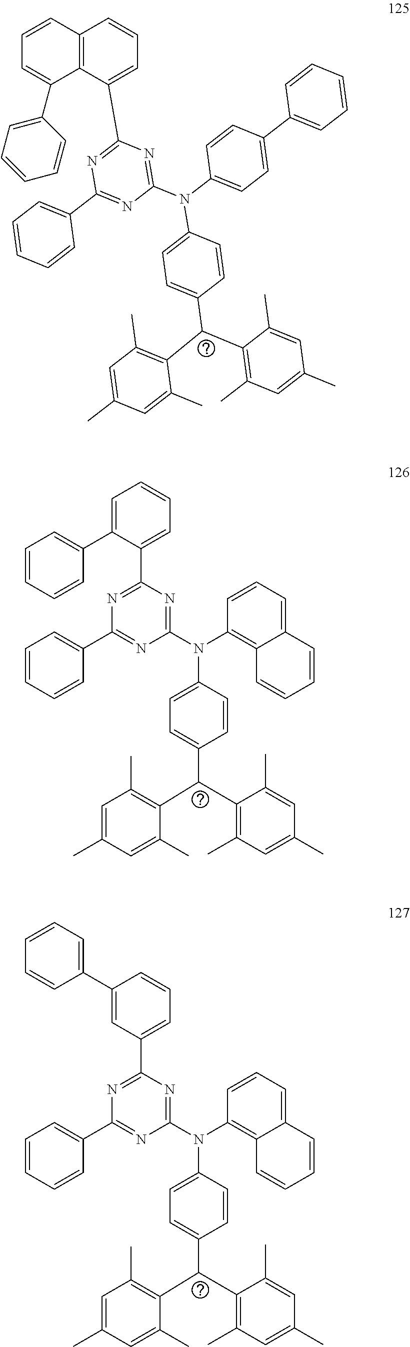 [SMILES: Cc1cc(C)c(C(c2ccc(N(c3ccc(-c4ccccc4)cc3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3cccc4ccccc34)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3cccc4ccccc34)cc2)c2c(C)cc(C)cc2C)c(C)c1] 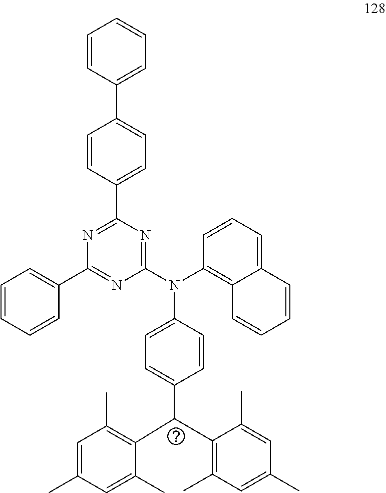 [SMILES: Cc1cc(C)c(C(c2ccc(N(c3nc(-c4ccccc4)nc(-c4ccc(-c5ccccc5)cc4)n3)c3cccc4ccccc34)cc2)c2c(C)cc(C)cc2C)c(C)c1] 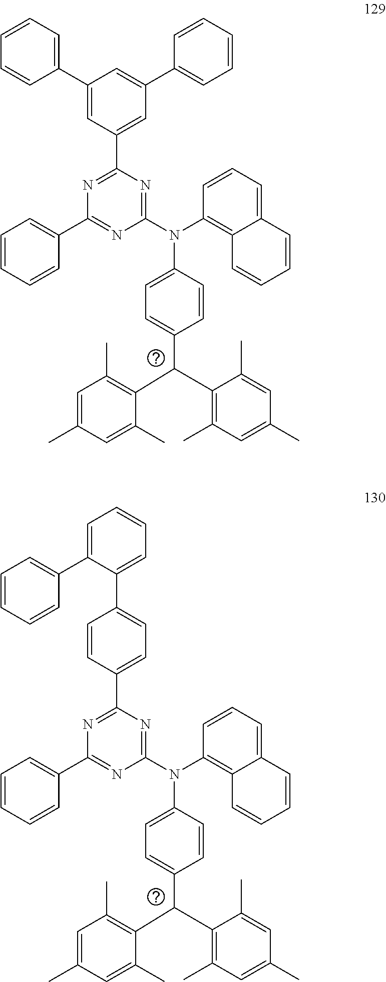 [SMILES: Cc1cc(C)c(C(c2ccc(N(c3nc(-c4ccccc4)nc(-c4cc(-c5ccccc5)cc(-c5ccccc5)c4)n3)c3cccc4ccccc34)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3cccc4ccccc34)cc2)c2c(C)cc(C)cc2C)c(C)c1] 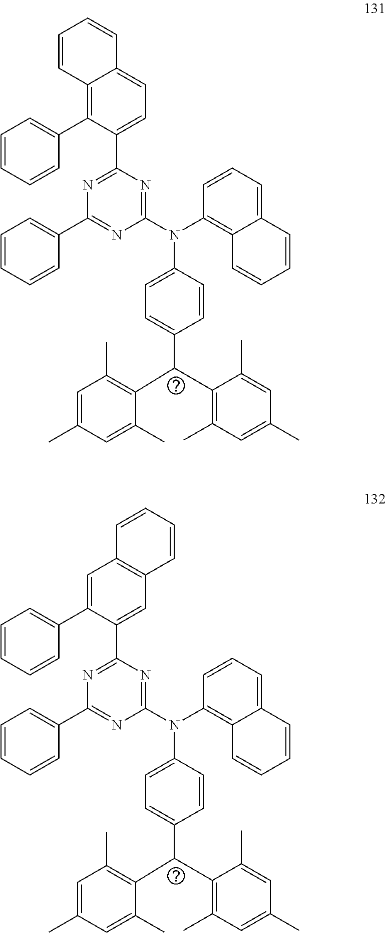 [SMILES: Cc1cc(C)c(C(c2ccc(N(c3nc(-c4ccccc4)nc(-c4ccc5ccccc5c4-c4ccccc4)n3)c3cccc4ccccc34)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3cccc4ccccc34)cc2)c2c(C)cc(C)cc2C)c(C)c1] 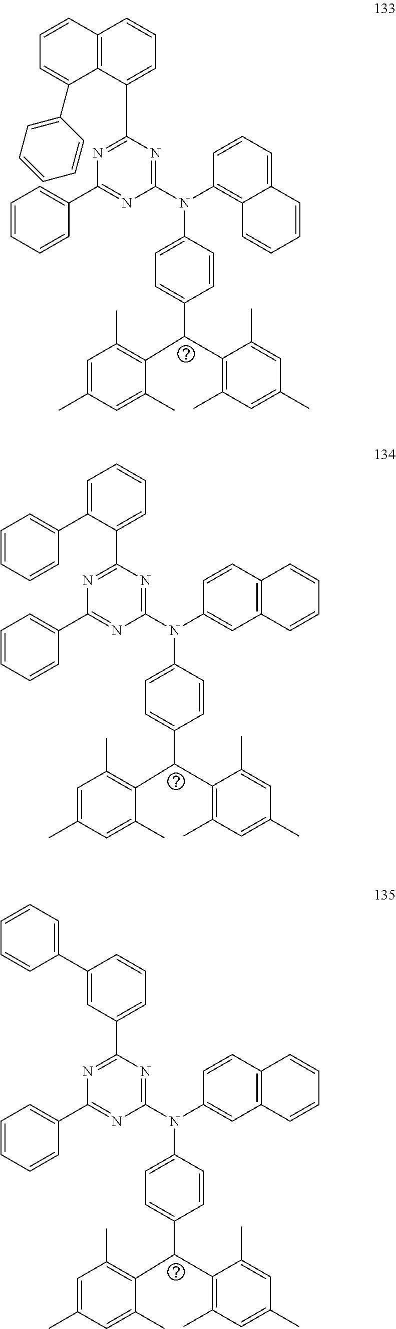 [SMILES: Cc1cc(C)c(C(c2ccc(N(c3nc(-c4ccccc4)nc(-c4cccc5cccc(-c6ccccc6)c45)n3)c3cccc4ccccc34)cc2)c2c(C)cc(C)cc2C)c(C)c1 ; Cc1cc(C)c(C(c2ccc(N(c3ccc4ccccc4c3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cccc(-c5ccccc5)c4)n3)cc2)c2c(C)cc(C)cc2C)c(C)c1] 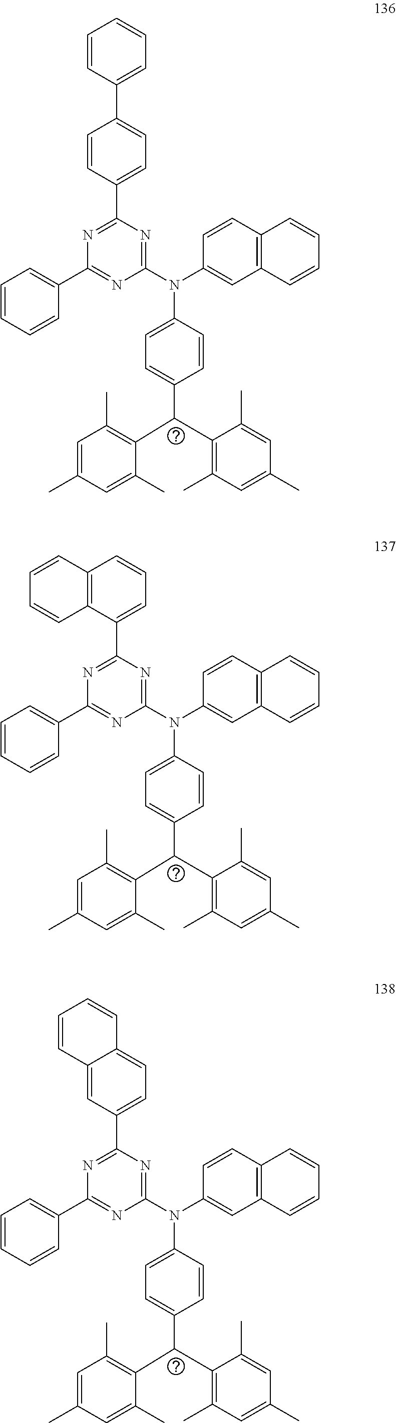 [SMILES: Cc1cc(C)c(C(c2ccc(N(c3ccc4ccccc4c3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cccc5ccccc45)n3)cc2)c2c(C)cc(C)cc2C)c(C)c1 ; Cc1cc(C)c(C(c2ccc(N(c3ccc4ccccc4c3)c3nc(-c4ccccc4)nc(-c4ccc5ccccc5c4)n3)cc2)c2c(C)cc(C)cc2C)c(C)c1] 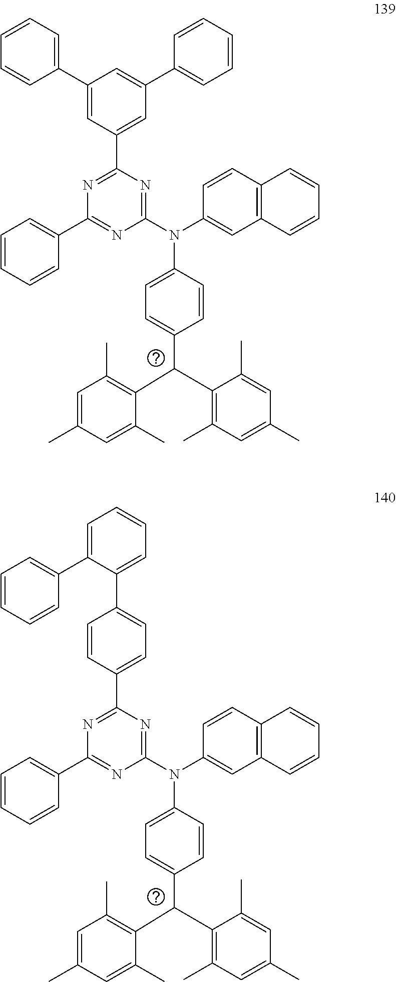 [SMILES: Cc1cc(C)c(C(c2ccc(N(c3ccc4ccccc4c3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc4ccccc4c3)c3nc(-c4ccccc4)nc(-c4ccc(-c5ccccc5-c5ccccc5)cc4)n3)cc2)c2c(C)cc(C)cc2C)c(C)c1] 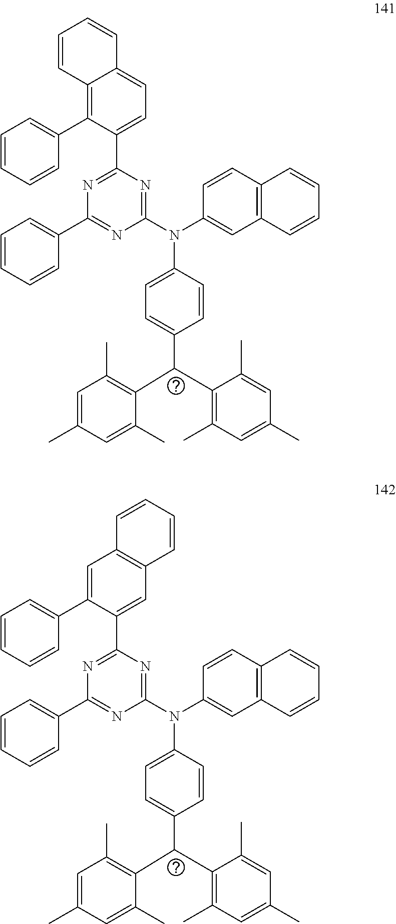 [SMILES: Cc1cc(C)c(C(c2ccc(N(c3ccc4ccccc4c3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc4ccccc4c3)c3nc(-c4ccccc4)nc(-c4cc5ccccc5cc4-c4ccccc4)n3)cc2)c2c(C)cc(C)cc2C)c(C)c1] 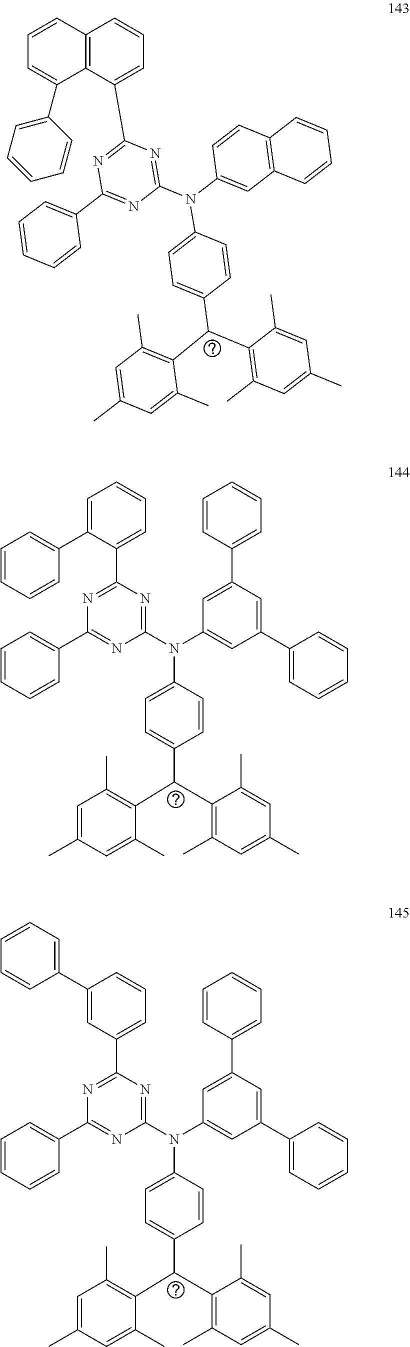 [SMILES: Cc1cc(C)c(C(c2ccc(N(c3ccc4ccccc4c3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4cccc(-c5ccccc5)c4)n3)cc2)c2c(C)cc(C)cc2C)c(C)c1] 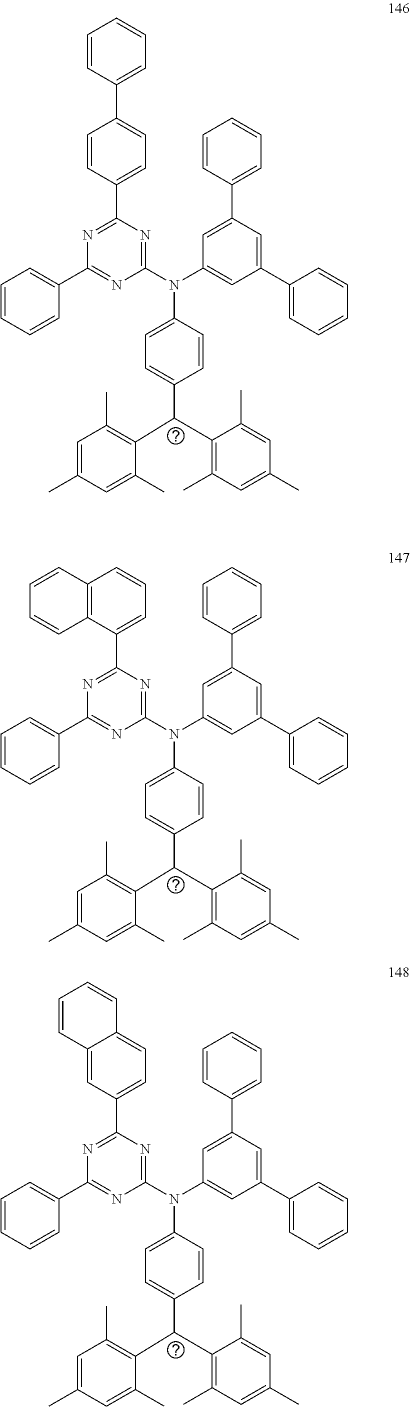 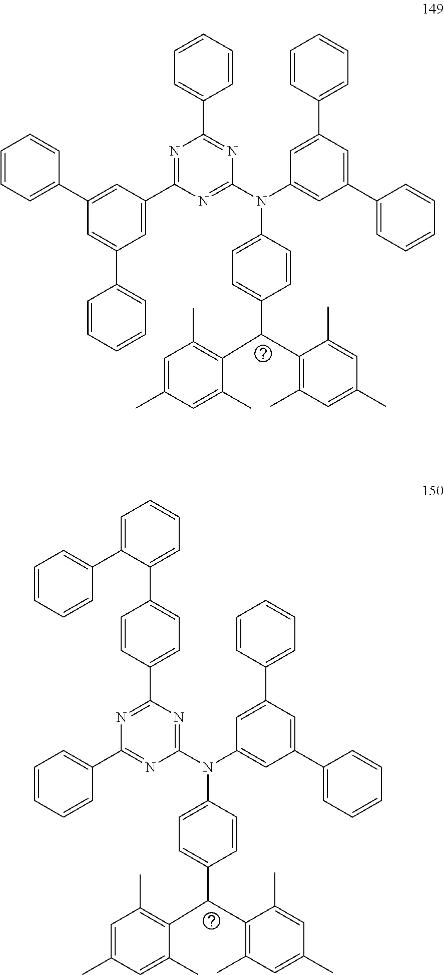 [SMILES: Cc1cc(C)c(C(c2ccc(N(c3cc(-c4ccccc4)cc(-c4ccccc4)c3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4ccc(-c5ccccc5-c5ccccc5)cc4)n3)cc2)c2c(C)cc(C)cc2C)c(C)c1] 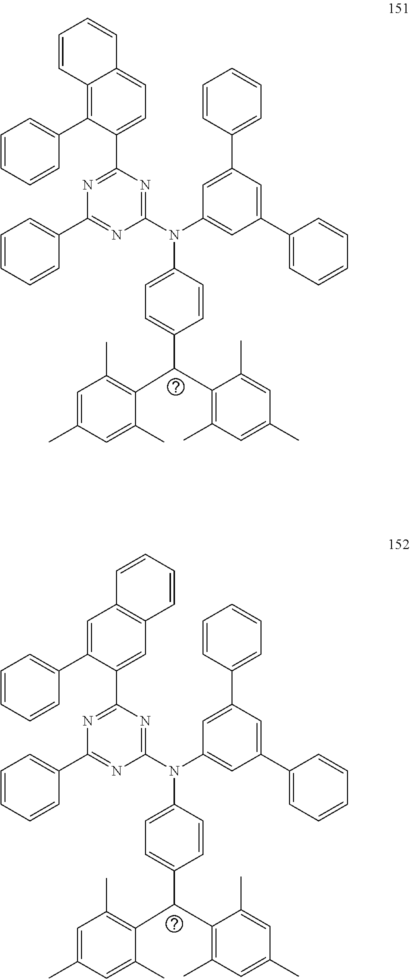 [SMILES: Cc1cc(C)c(C(c2ccc(N(c3cc(-c4ccccc4)cc(-c4ccccc4)c3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3cc(-c4ccccc4)cc(-c4ccccc4)c3)c3nc(-c4ccccc4)nc(-c4cc5ccccc5cc4-c4ccccc4)n3)cc2)c2c(C)cc(C)cc2C)c(C)c1] 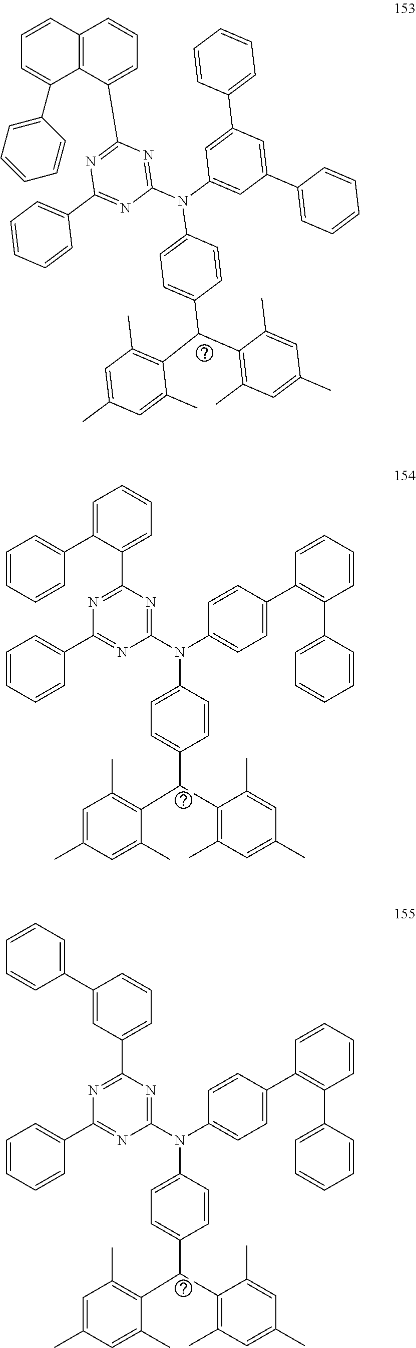 [SMILES: Cc1cc(C)c(C(c2ccc(N(c3cc(-c4ccccc4)cc(-c4ccccc4)c3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cccc(-c5ccccc5)c4)n3)cc2)c2c(C)cc(C)cc2C)c(C)c1] 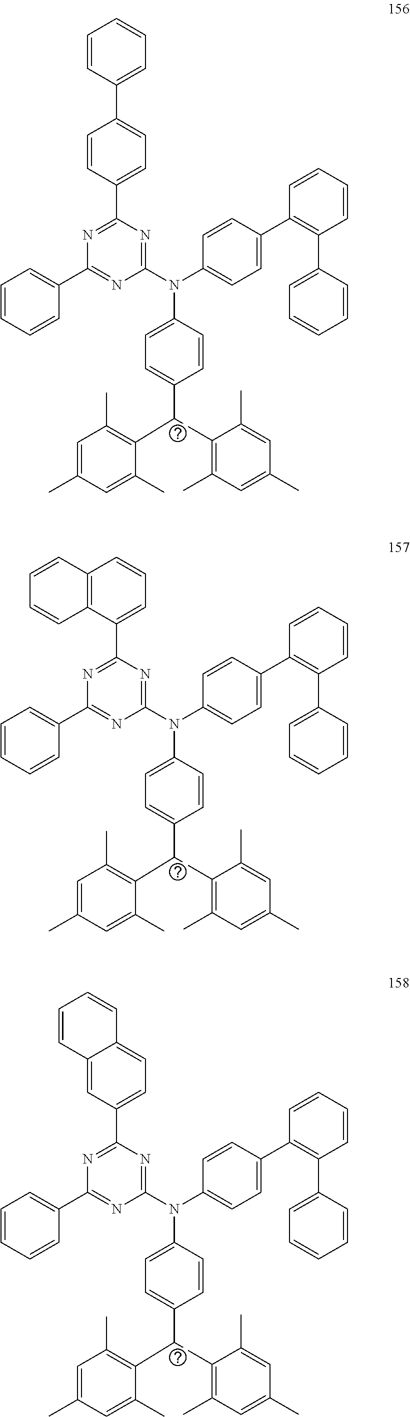 [SMILES: Cc1cc(C)c(C(c2ccc(N(c3ccc(-c4ccccc4-c4ccccc4)cc3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cccc5ccccc45)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccc5ccccc5c4)n3)cc2)c2c(C)cc(C)cc2C)c(C)c1] 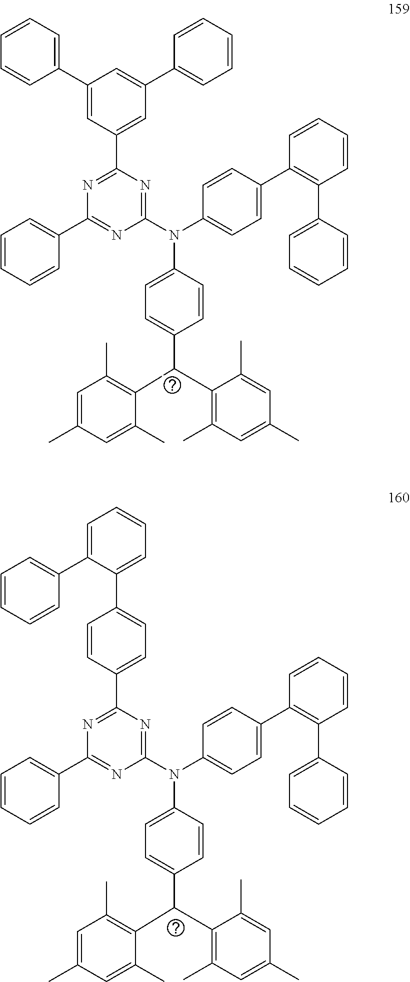 [SMILES: Cc1cc(C)c(C(c2ccc(N(c3ccc(-c4ccccc4-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4ccc(-c5ccccc5-c5ccccc5)cc4)n3)cc2)c2c(C)cc(C)cc2C)c(C)c1] 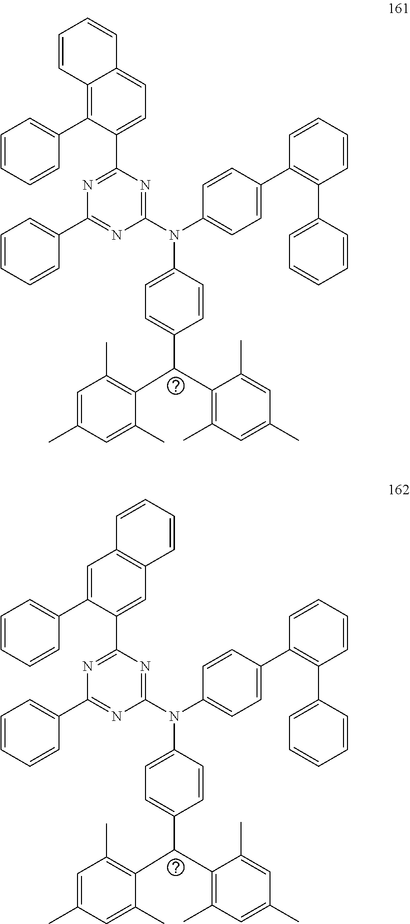 [SMILES: Cc1cc(C)c(C(c2ccc(N(c3ccc(-c4ccccc4-c4ccccc4)cc3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3ccc(-c4ccccc4-c4ccccc4)cc3)c3nc(-c4ccccc4)nc(-c4cc5ccccc5cc4-c4ccccc4)n3)cc2)c2c(C)cc(C)cc2C)c(C)c1] 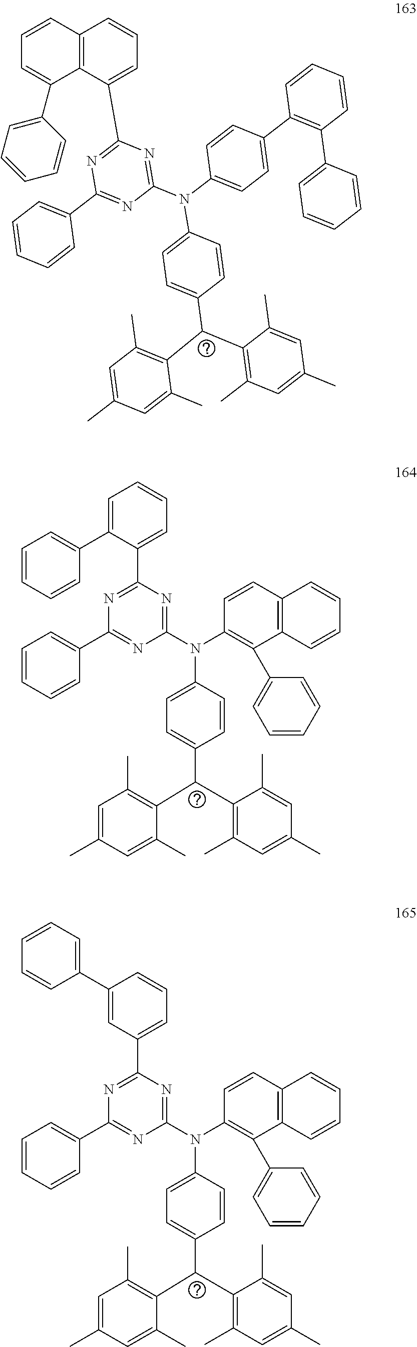 [SMILES: Cc1cc(C)c(C(c2ccc(N(c3ccc(-c4ccccc4-c4ccccc4)cc3)c3nc(-c4ccccc4)nc(-c4cccc5cccc(-c6ccccc6)c45)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3ccc4ccccc4c3-c3ccccc3)cc2)c2c(C)cc(C)cc2C)c(C)c1] 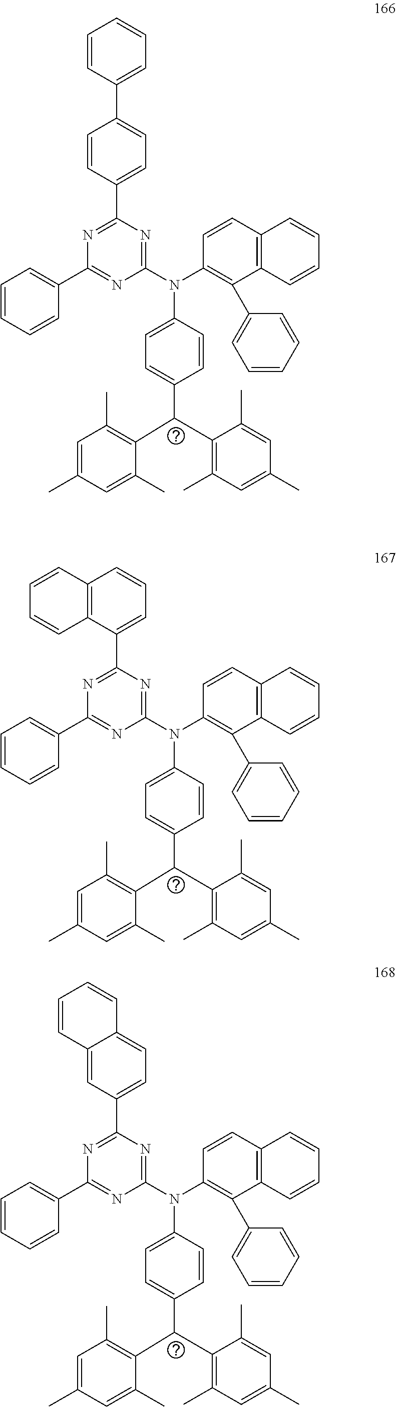 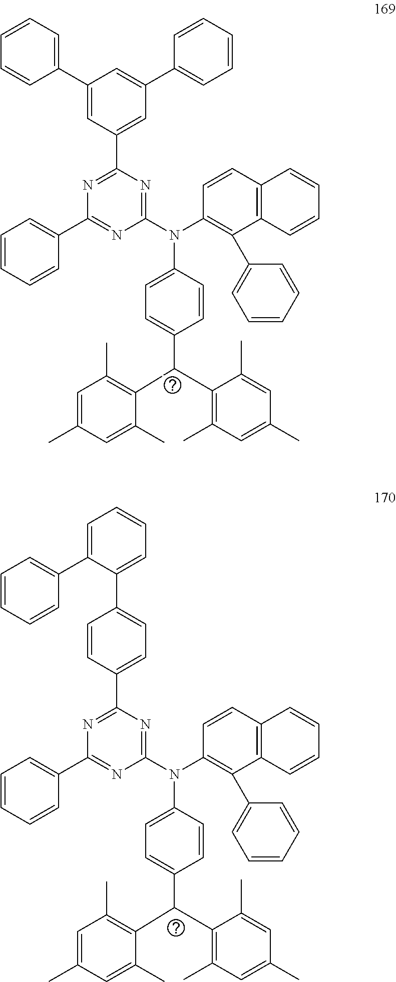 [SMILES: Cc1cc(C)c(C(c2ccc(N(c3nc(-c4ccccc4)nc(-c4cc(-c5ccccc5)cc(-c5ccccc5)c4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3ccc4ccccc4c3-c3ccccc3)cc2)c2c(C)cc(C)cc2C)c(C)c1] 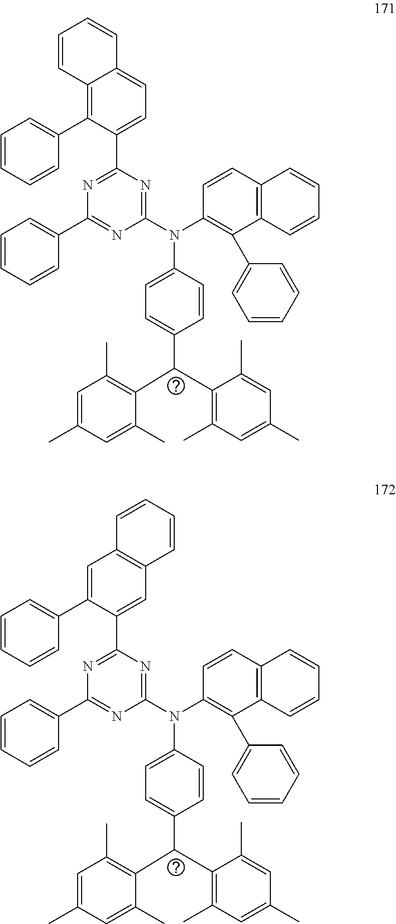 [SMILES: Cc1cc(C)c(C(c2ccc(N(c3nc(-c4ccccc4)nc(-c4ccc5ccccc5c4-c4ccccc4)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3ccc4ccccc4c3-c3ccccc3)cc2)c2c(C)cc(C)cc2C)c(C)c1] 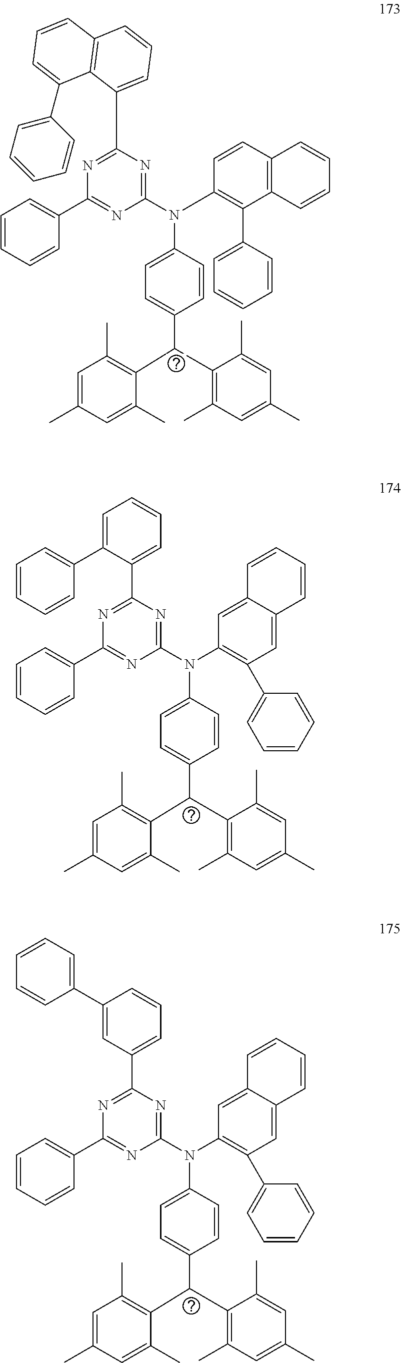 [SMILES: Cc1cc(C)c(C(c2ccc(N(c3nc(-c4ccccc4)nc(-c4cccc5cccc(-c6ccccc6)c45)n3)c3ccc4ccccc4c3-c3ccccc3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3cc4ccccc4cc3-c3ccccc3)cc2)c2c(C)cc(C)cc2C)c(C)c1] 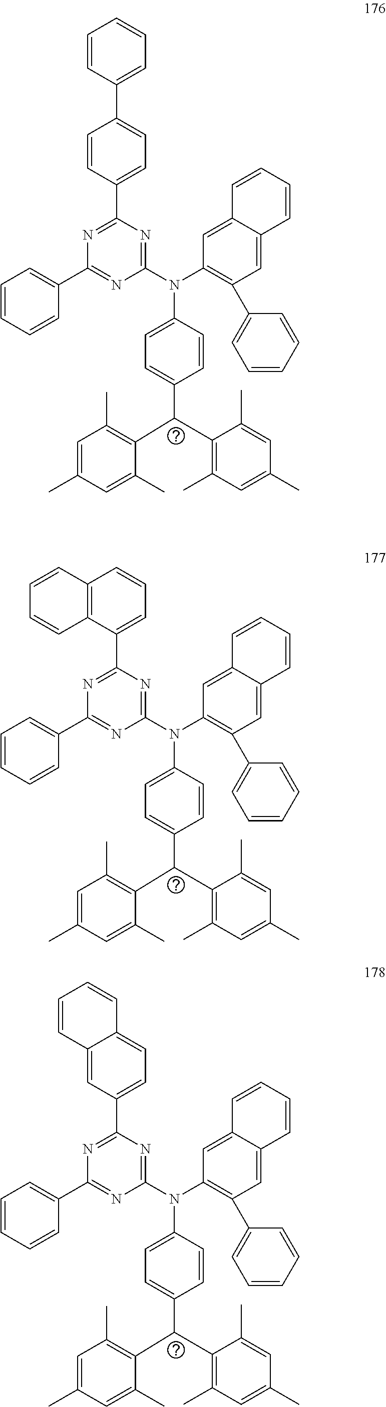 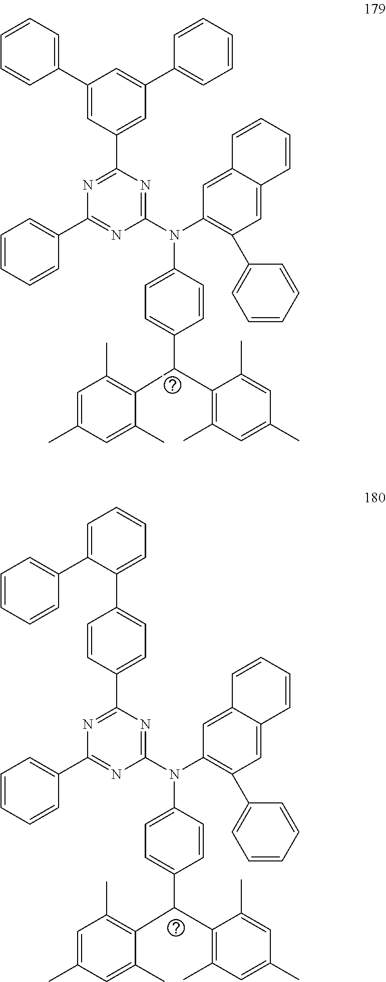 [SMILES: Cc1cc(C)c(C(c2ccc(N(c3nc(-c4ccccc4)nc(-c4cc(-c5ccccc5)cc(-c5ccccc5)c4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4ccc(-c5ccccc5-c5ccccc5)cc4)n3)c3cc4ccccc4cc3-c3ccccc3)cc2)c2c(C)cc(C)cc2C)c(C)c1] 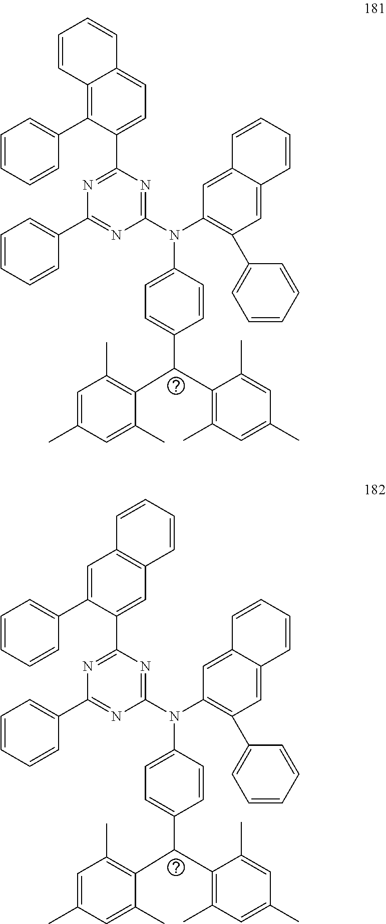 [SMILES: Cc1cc(C)c(C(c2ccc(N(c3nc(-c4ccccc4)nc(-c4ccc5ccccc5c4-c4ccccc4)n3)c3cc4ccccc4cc3-c3ccccc3)cc2)c2c(C)cc(C)cc2C)c(C)c1 ; Cc1cc(C)c(C(c2ccc(N(c3nc(-c4ccccc4)nc(-c4cc5ccccc5cc4-c4ccccc4)n3)c3cc4ccccc4cc3-c3ccccc3)cc2)c2c(C)cc(C)cc2C)c(C)c1] 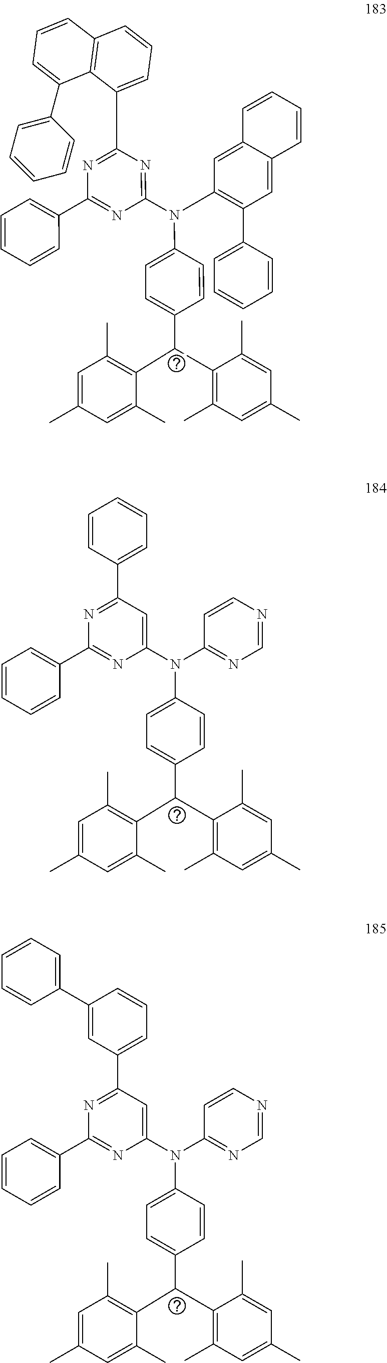 [SMILES: Cc1cc(C)c(C(c2ccc(N(c3nc(-c4ccccc4)nc(-c4cccc5cccc(-c6ccccc6)c45)n3)c3cc4ccccc4cc3-c3ccccc3)cc2)c2c(C)cc(C)cc2C)c(C)c1 ; Cc1cc(C)c(C(c2ccc(N(c3ccncn3)c3cc(-c4ccccc4)nc(-c4ccccc4)n3)cc2)c2c(C)cc(C)cc2C)c(C)c1 ; Cc1cc(C)c(C(c2ccc(N(c3ccncn3)c3cc(-c4cccc(-c5ccccc5)c4)nc(-c4ccccc4)n3)cc2)c2c(C)cc(C)cc2C)c(C)c1] 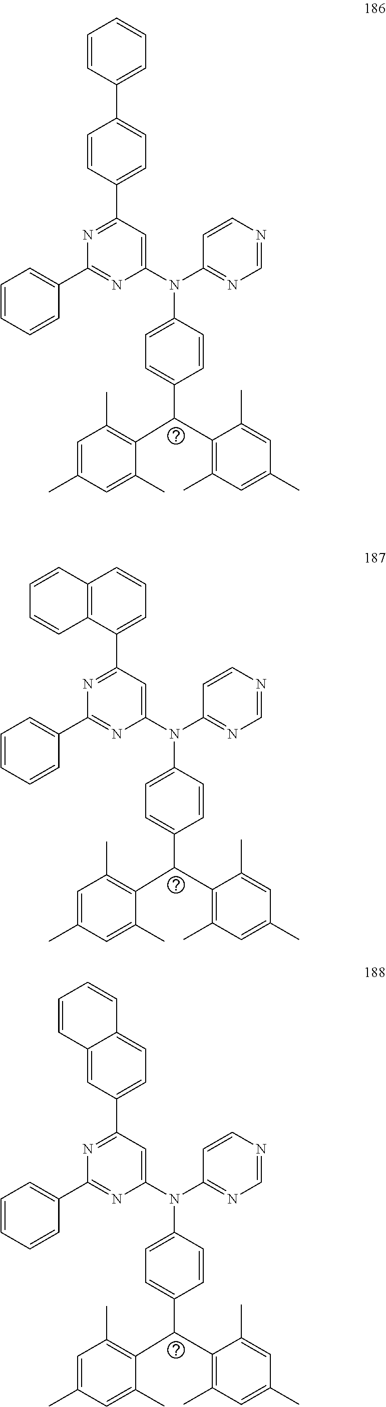 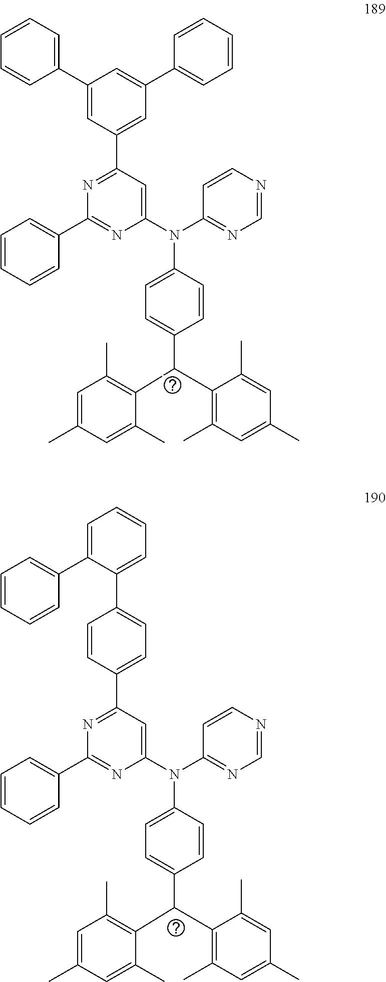 [SMILES: Cc1cc(C)c(C(c2ccc(N(c3ccncn3)c3cc(-c4cc(-c5ccccc5)cc(-c5ccccc5)c4)nc(-c4ccccc4)n3)cc2)c2c(C)cc(C)cc2C)c(C)c1 ; Cc1cc(C)c(C(c2ccc(N(c3ccncn3)c3cc(-c4ccc(-c5ccccc5-c5ccccc5)cc4)nc(-c4ccccc4)n3)cc2)c2c(C)cc(C)cc2C)c(C)c1] 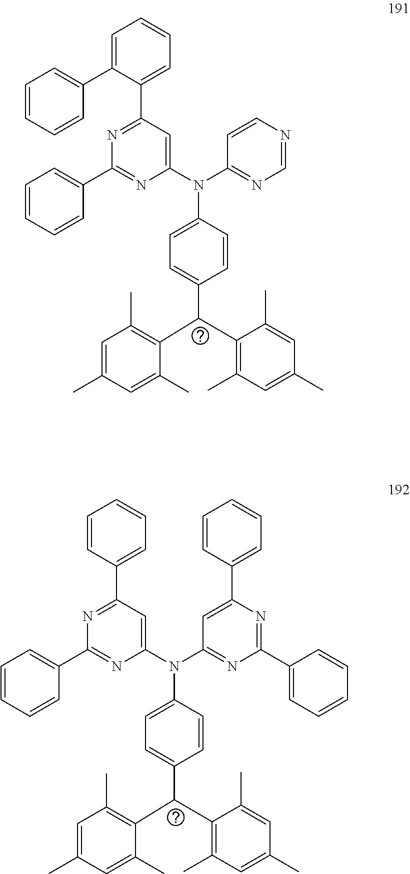 [SMILES: Cc1cc(C)c(C(c2ccc(N(c3ccncn3)c3cc(-c4ccccc4-c4ccccc4)nc(-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccccc4)nc(-c4ccccc4)n3)cc2)c2c(C)cc(C)cc2C)c(C)c1] 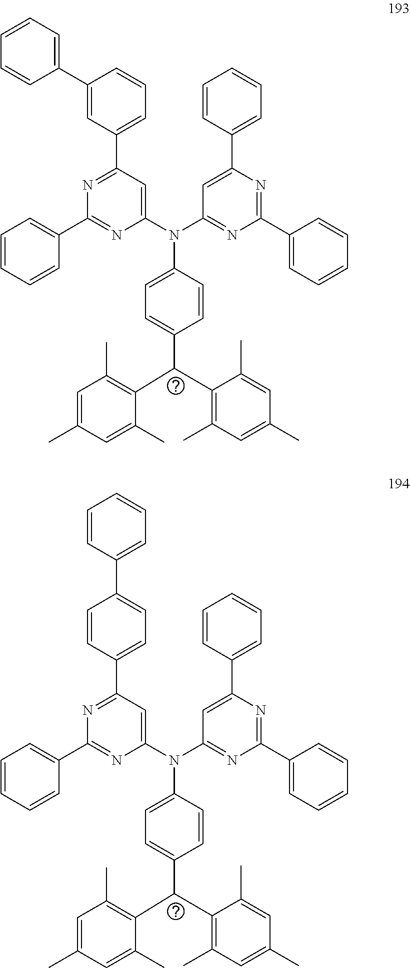 [SMILES: Cc1cc(C)c(C(c2ccc(N(c3cc(-c4ccccc4)nc(-c4ccccc4)n3)c3cc(-c4cccc(-c5ccccc5)c4)nc(-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccc(-c5ccccc5)cc4)nc(-c4ccccc4)n3)cc2)c2c(C)cc(C)cc2C)c(C)c1] 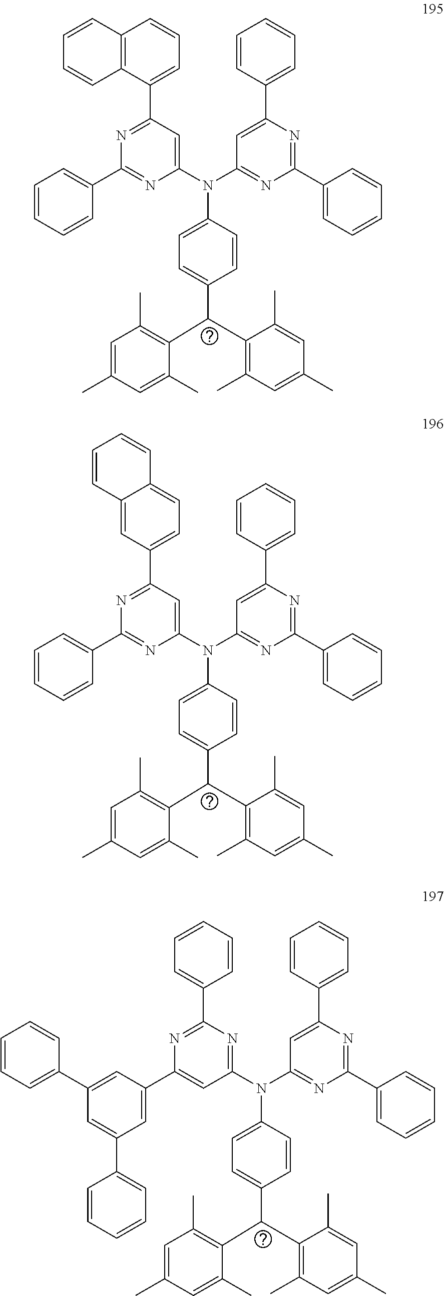 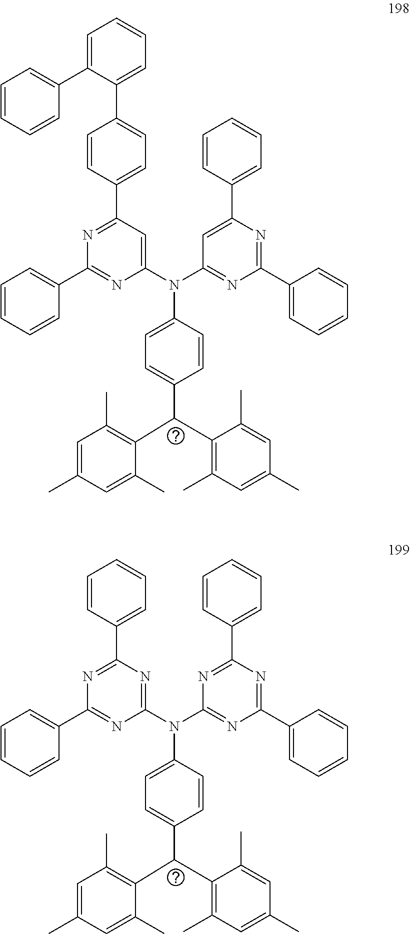 [SMILES: Cc1cc(C)c(C(c2ccc(N(c3cc(-c4ccccc4)nc(-c4ccccc4)n3)c3cc(-c4ccc(-c5ccccc5-c5ccccc5)cc4)nc(-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccccc4)n3)cc2)c2c(C)cc(C)cc2C)c(C)c1] 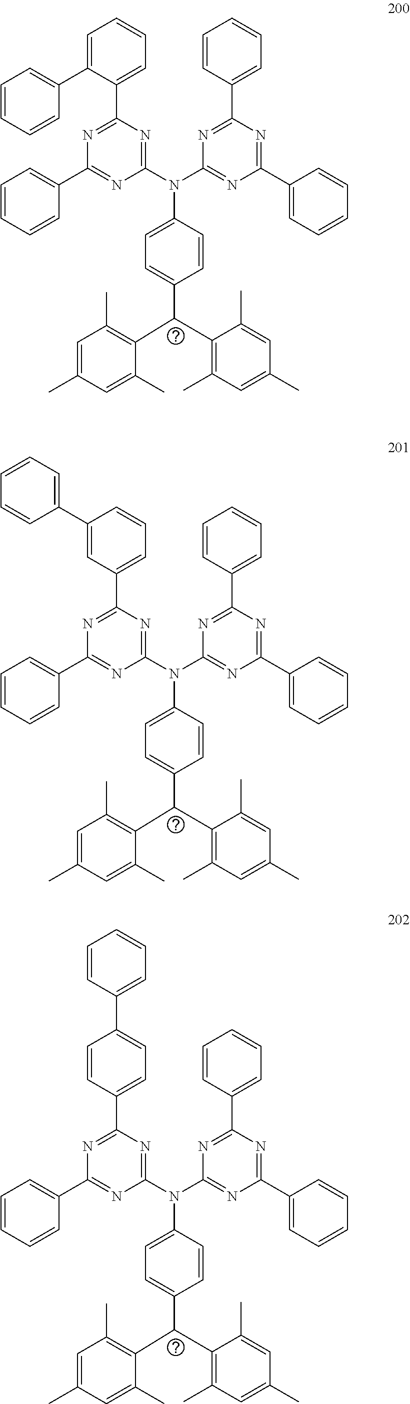 [SMILES: Cc1cc(C)c(C(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc(-c5ccccc5)cc4)n3)cc2)c2c(C)cc(C)cc2C)c(C)c1] 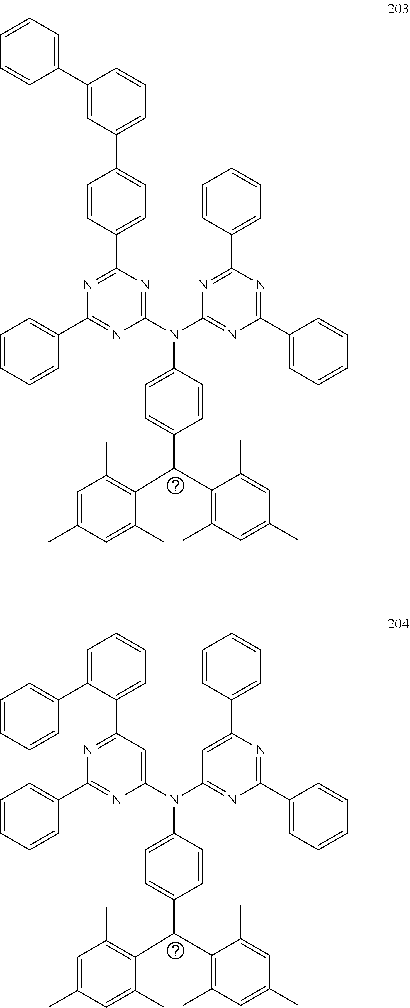 [SMILES: Cc1cc(C)c(C(c2ccc(N(c3nc(-c4ccccc4)nc(-c4ccccc4)n3)c3nc(-c4ccccc4)nc(-c4ccc(-c5cccc(-c6ccccc6)c5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3cc(-c4ccccc4-c4ccccc4)nc(-c4ccccc4)n3)cc2)c2c(C)cc(C)cc2C)c(C)c1] 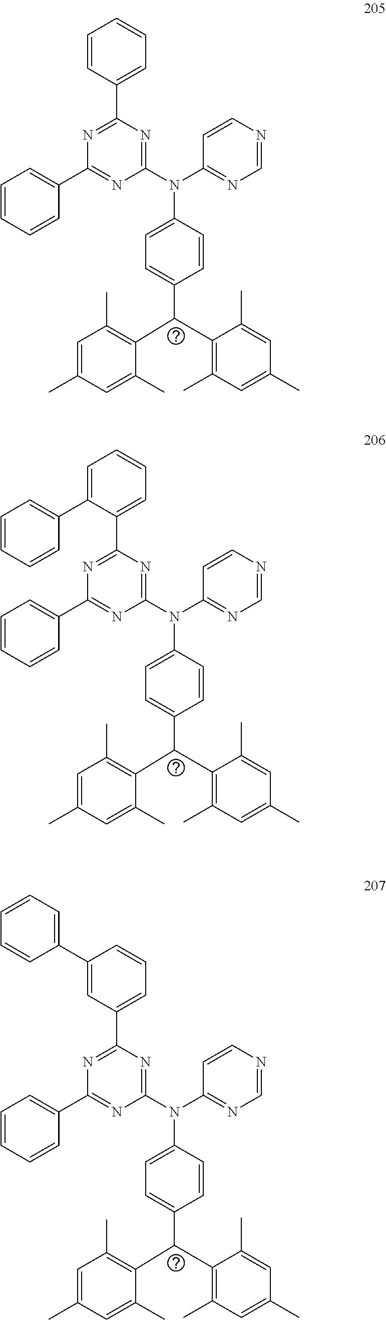 [SMILES: Cc1cc(C)c(C(c2ccc(N(c3ccncn3)c3nc(-c4ccccc4)nc(-c4ccccc4)n3)cc2)c2c(C)cc(C)cc2C)c(C)c1 ; Cc1cc(C)c(C(c2ccc(N(c3ccncn3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3ccncn3)c3nc(-c4ccccc4)nc(-c4cccc(-c5ccccc5)c4)n3)cc2)c2c(C)cc(C)cc2C)c(C)c1] 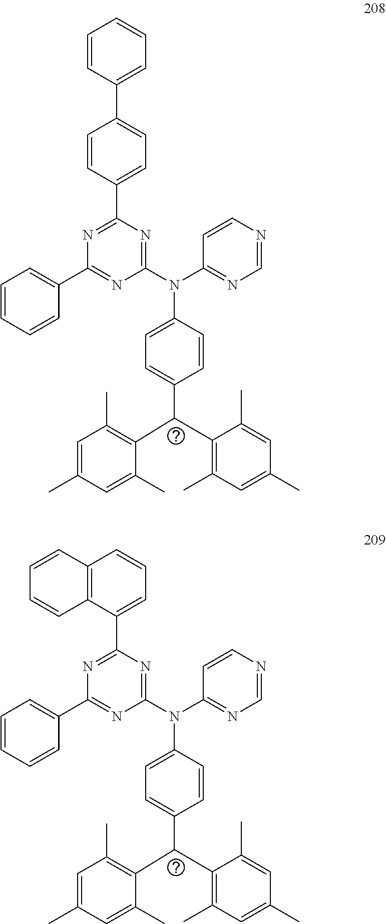 [SMILES: Cc1cc(C)c(C(c2ccc(N(c3ccncn3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3ccncn3)c3nc(-c4ccccc4)nc(-c4cccc5ccccc45)n3)cc2)c2c(C)cc(C)cc2C)c(C)c1] 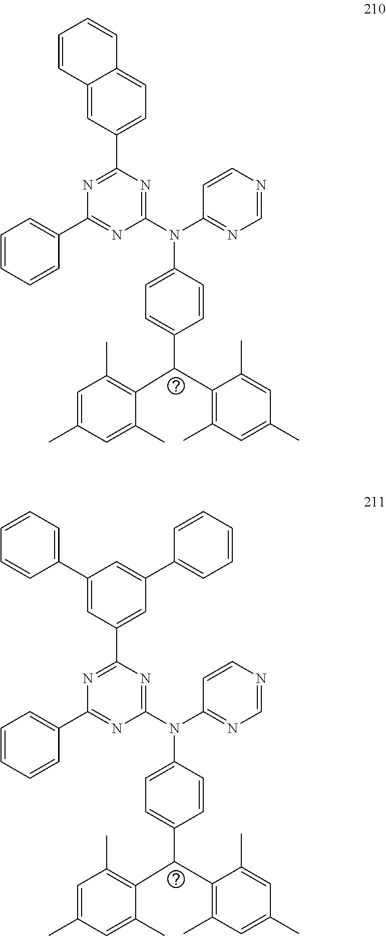 [SMILES: Cc1cc(C)c(C(c2ccc(N(c3ccncn3)c3nc(-c4ccccc4)nc(-c4ccc5ccccc5c4)n3)cc2)c2c(C)cc(C)cc2C)c(C)c1 ; Cc1cc(C)c(C(c2ccc(N(c3ccncn3)c3nc(-c4ccccc4)nc(-c4cc(-c5ccccc5)cc(-c5ccccc5)c4)n3)cc2)c2c(C)cc(C)cc2C)c(C)c1] 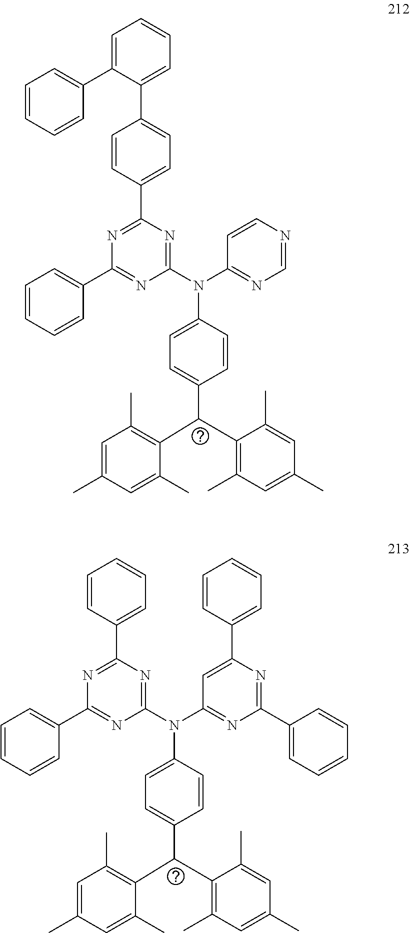 [SMILES: Cc1cc(C)c(C(c2ccc(N(c3ccncn3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccccc4)n3)cc2)c2c(C)cc(C)cc2C)c(C)c1] 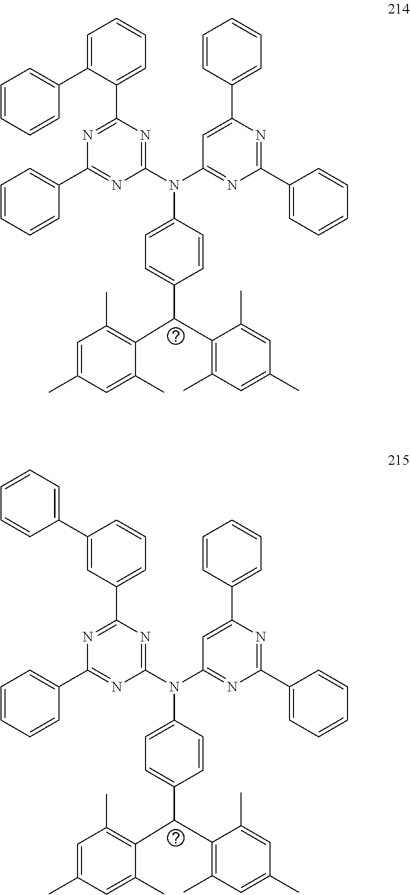 [SMILES: Cc1cc(C)c(C(c2ccc(N(c3cc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1] 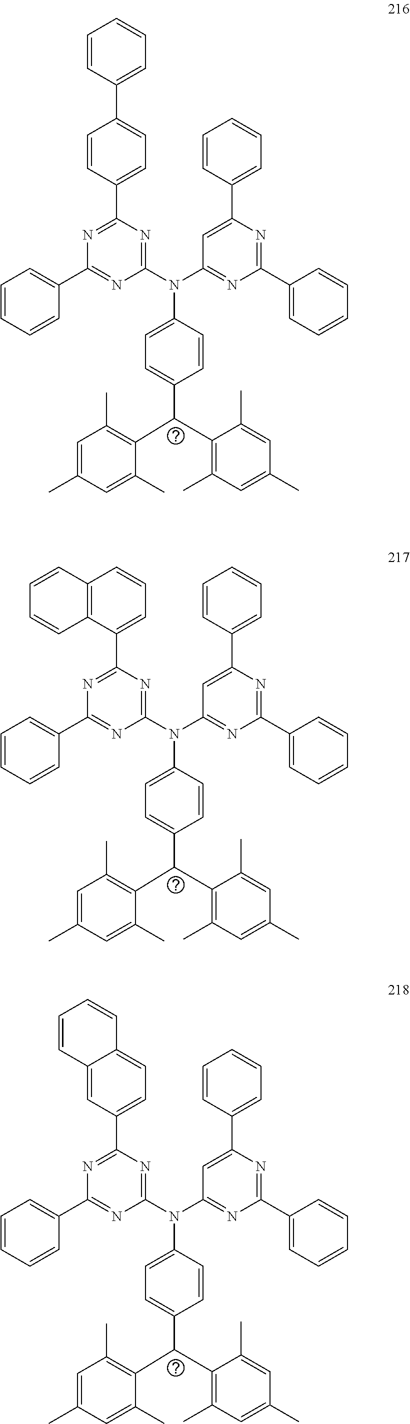 [SMILES: Cc1cc(C)c(C(c2ccc(N(c3cc(-c4ccccc4)nc(-c4ccccc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccc5ccccc5c4)n3)cc2)c2c(C)cc(C)cc2C)c(C)c1] 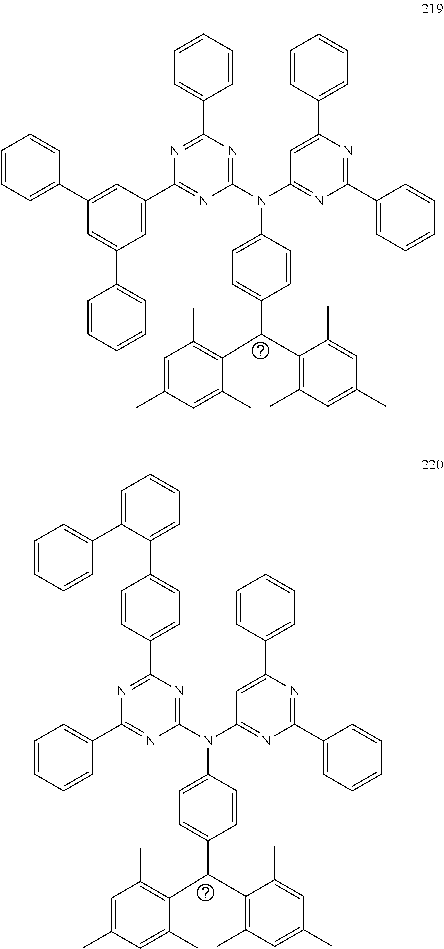 [SMILES: Cc1cc(C)c(C(c2ccc(N(c3cc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3cc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1] 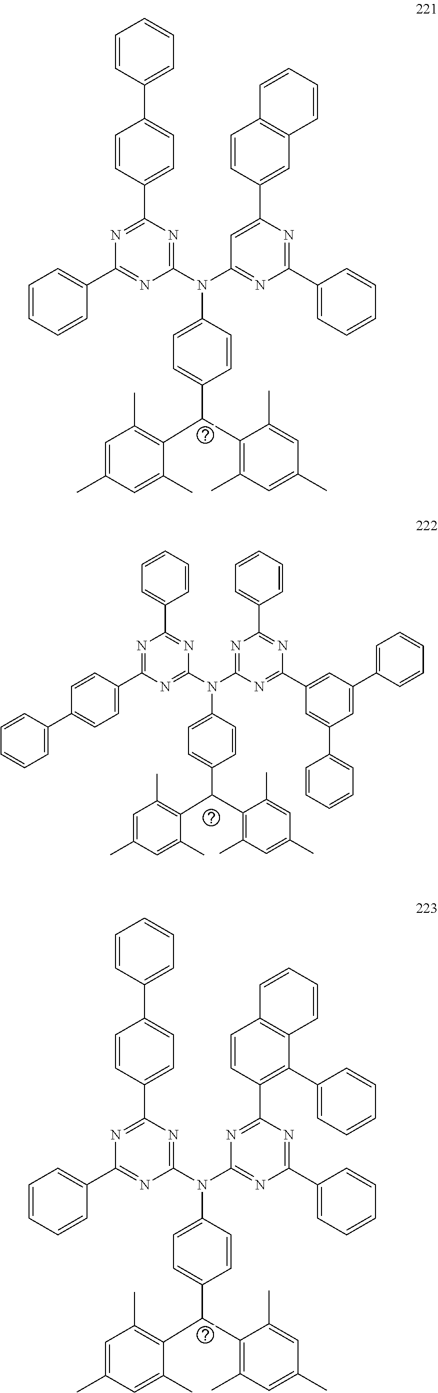 [SMILES: Cc1cc(C)c(C(c2ccc(N(c3cc(-c4ccc5ccccc5c4)nc(-c4ccccc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4ccc5ccccc5c4-c4ccccc4)n3)cc2)c2c(C)cc(C)cc2C)c(C)c1] 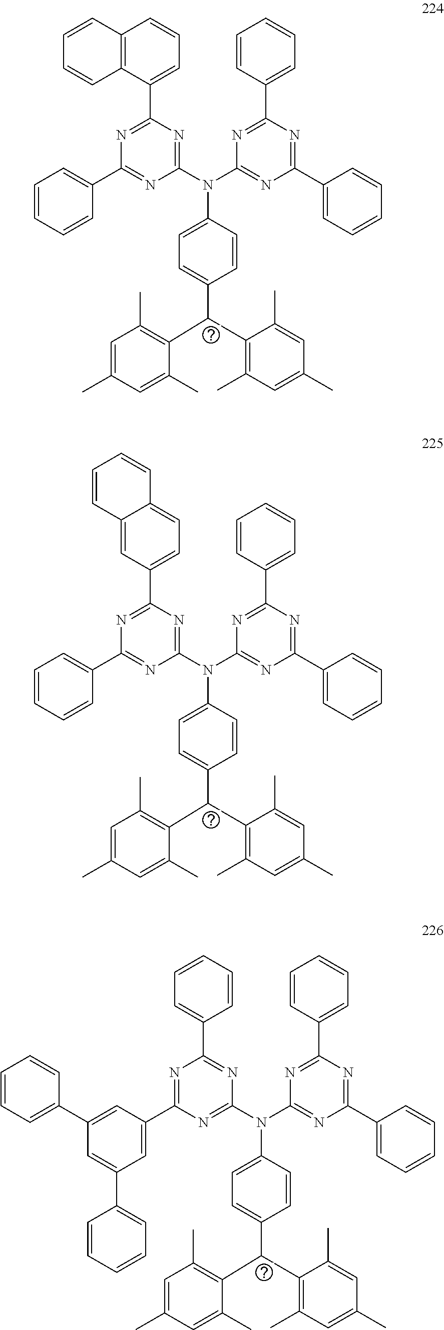 [SMILES: Cc1cc(C)c(C(c2ccc(N(c3nc(-c4ccccc4)nc(-c4ccccc4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc5ccccc5c4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1] 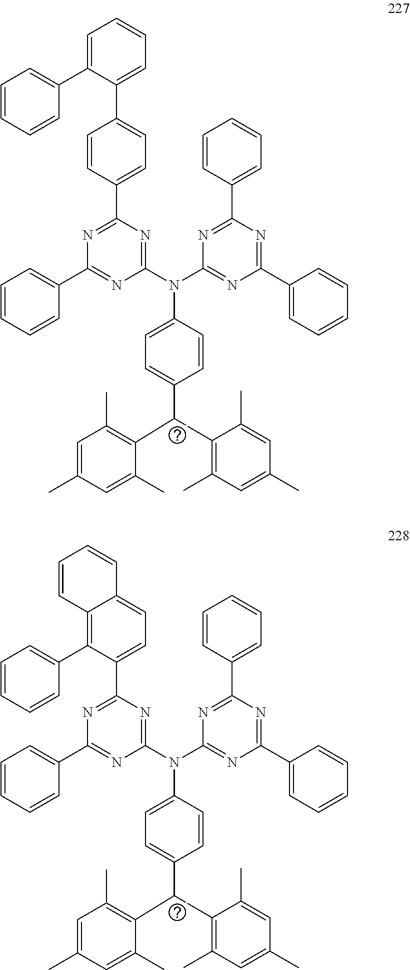 [SMILES: Cc1cc(C)c(C(c2ccc(N(c3nc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccc5ccccc5c4-c4ccccc4)n3)cc2)c2c(C)cc(C)cc2C)c(C)c1] 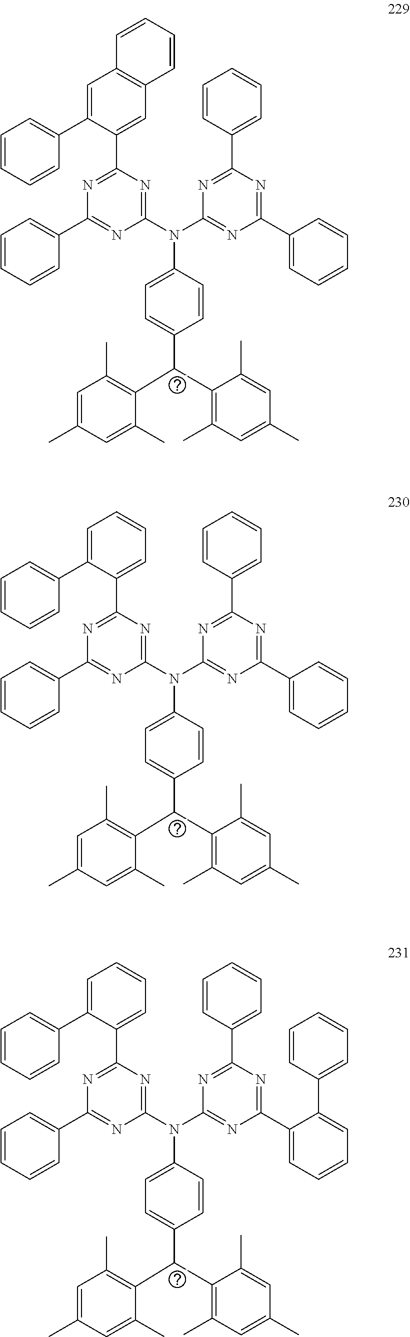 [SMILES: Cc1cc(C)c(C(c2ccc(N(c3nc(-c4ccccc4)nc(-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)cc(C)cc2C)c(C)c1] 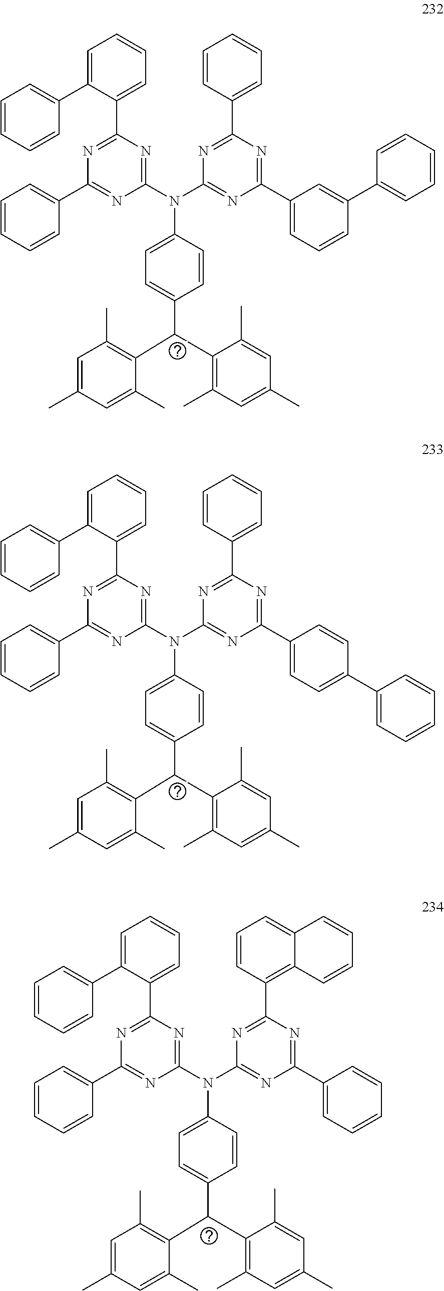 [SMILES: Cc1cc(C)c(C(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4cccc5ccccc45)n3)cc2)c2c(C)cc(C)cc2C)c(C)c1] 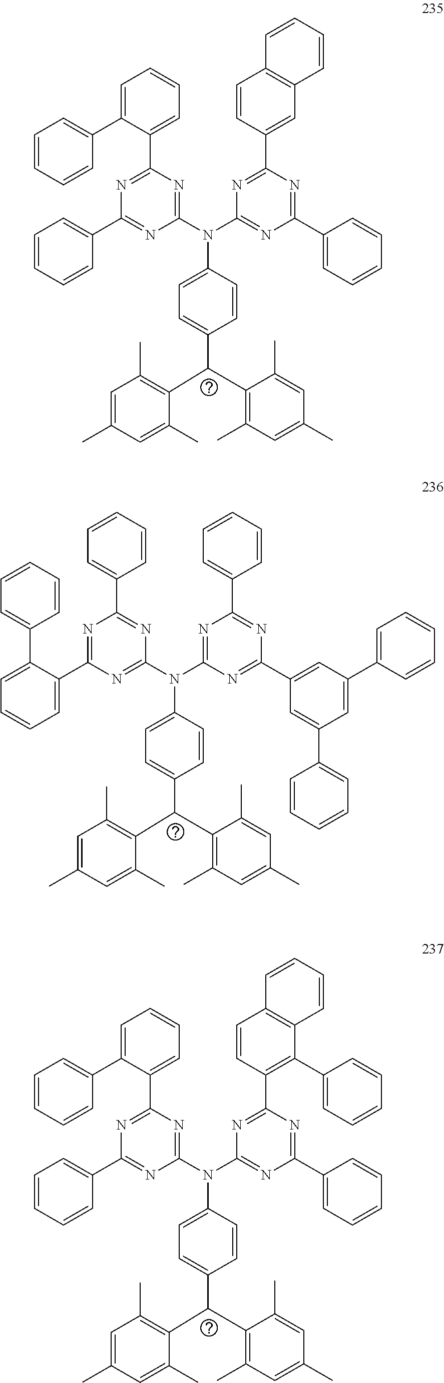 [SMILES: Cc1cc(C)c(C(c2ccc(N(c3nc(-c4ccccc4)nc(-c4ccc5ccccc5c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(C(c2ccc(N(c3nc(-c4ccccc4)nc(-c4ccccc4-c4ccccc4)n3)c3nc(-c4ccccc4)nc(-c4ccc5ccccc5c4-c4ccccc4)n3)cc2)c2c(C)cc(C)cc2C)c(C)c1] 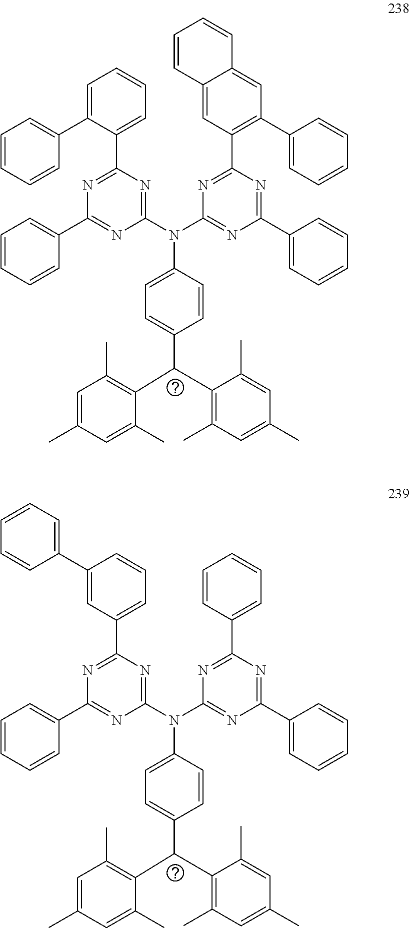 [SMILES: Cc1cc(C)c(C(c2ccc(N(c3nc(-c4ccccc4)nc(-c4ccccc4-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1] 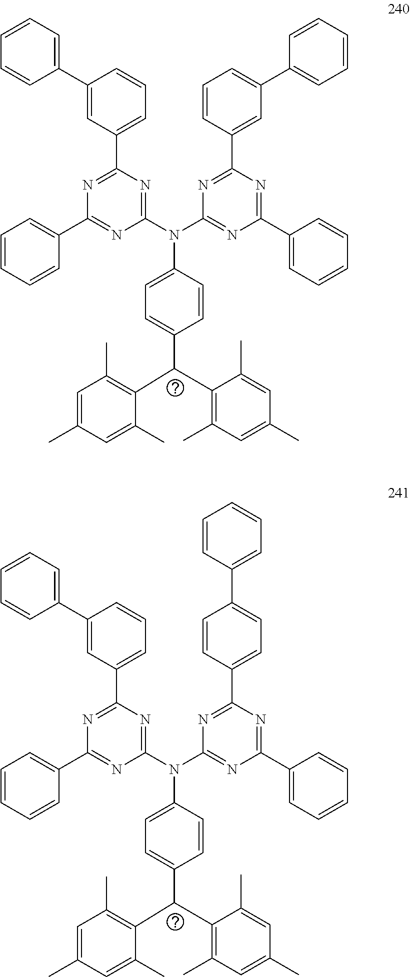 [SMILES: Cc1cc(C)c(C(c2ccc(N(c3nc(-c4ccccc4)nc(-c4cccc(-c5ccccc5)c4)n3)c3nc(-c4ccccc4)nc(-c4cccc(-c5ccccc5)c4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cccc(-c5ccccc5)c4)n3)cc2)c2c(C)cc(C)cc2C)c(C)c1] 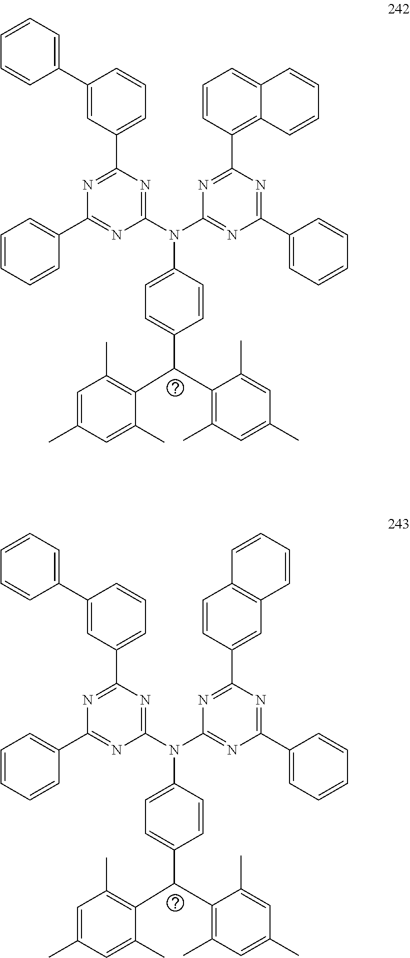 [SMILES: Cc1cc(C)c(C(c2ccc(N(c3nc(-c4ccccc4)nc(-c4cccc(-c5ccccc5)c4)n3)c3nc(-c4ccccc4)nc(-c4cccc5ccccc45)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4ccc5ccccc5c4)n3)cc2)c2c(C)cc(C)cc2C)c(C)c1] 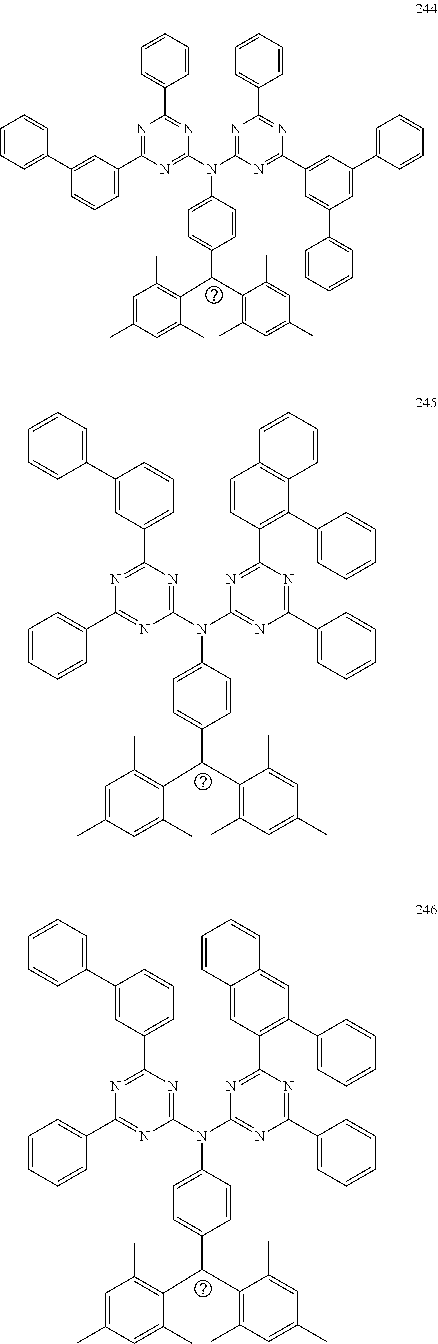 [SMILES: Cc1cc(C)c(C(c2ccc(N(c3nc(-c4ccccc4)nc(-c4cccc(-c5ccccc5)c4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4cccc(-c5ccccc5)c4)n3)c3nc(-c4ccccc4)nc(-c4cc5ccccc5cc4-c4ccccc4)n3)cc2)c2c(C)cc(C)cc2C)c(C)c1] 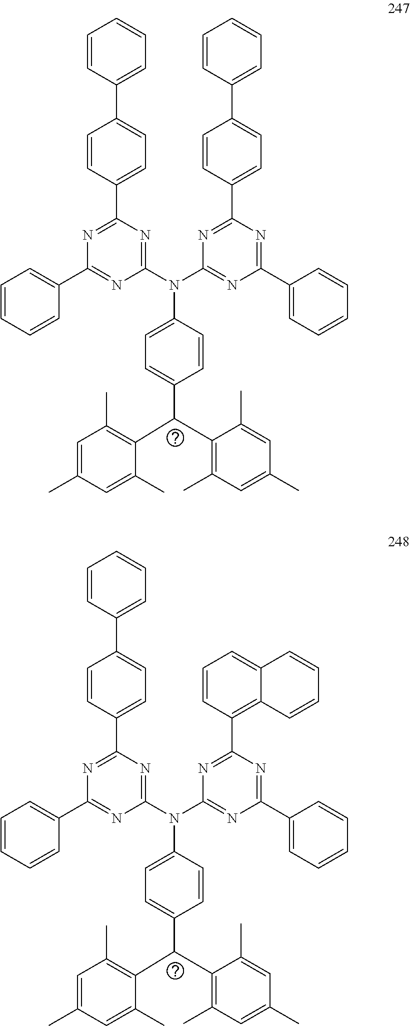 [SMILES: Cc1cc(C)c(C(c2ccc(N(c3nc(-c4ccccc4)nc(-c4ccc(-c5ccccc5)cc4)n3)c3nc(-c4ccccc4)nc(-c4ccc(-c5ccccc5)cc4)n3)cc2)c2c(C)cc(C)cc2C)c(C)c1 ; Cc1cc(C)c(C(c2ccc(N(c3nc(-c4ccccc4)nc(-c4ccc(-c5ccccc5)cc4)n3)c3nc(-c4ccccc4)nc(-c4cccc5ccccc45)n3)cc2)c2c(C)cc(C)cc2C)c(C)c1] 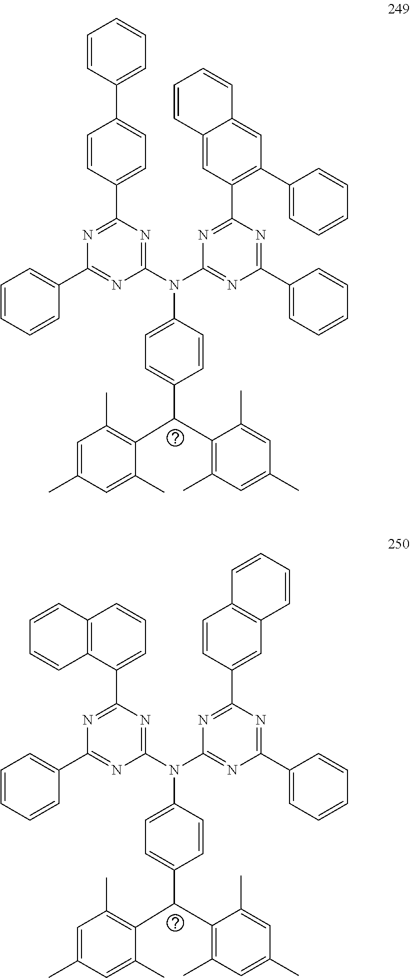 [SMILES: Cc1cc(C)c(C(c2ccc(N(c3nc(-c4ccccc4)nc(-c4ccc(-c5ccccc5)cc4)n3)c3nc(-c4ccccc4)nc(-c4cc5ccccc5cc4-c4ccccc4)n3)cc2)c2c(C)cc(C)cc2C)c(C)c1 ; Cc1cc(C)c(C(c2ccc(N(c3nc(-c4ccccc4)nc(-c4ccc5ccccc5c4)n3)c3nc(-c4ccccc4)nc(-c4cccc5ccccc45)n3)cc2)c2c(C)cc(C)cc2C)c(C)c1] 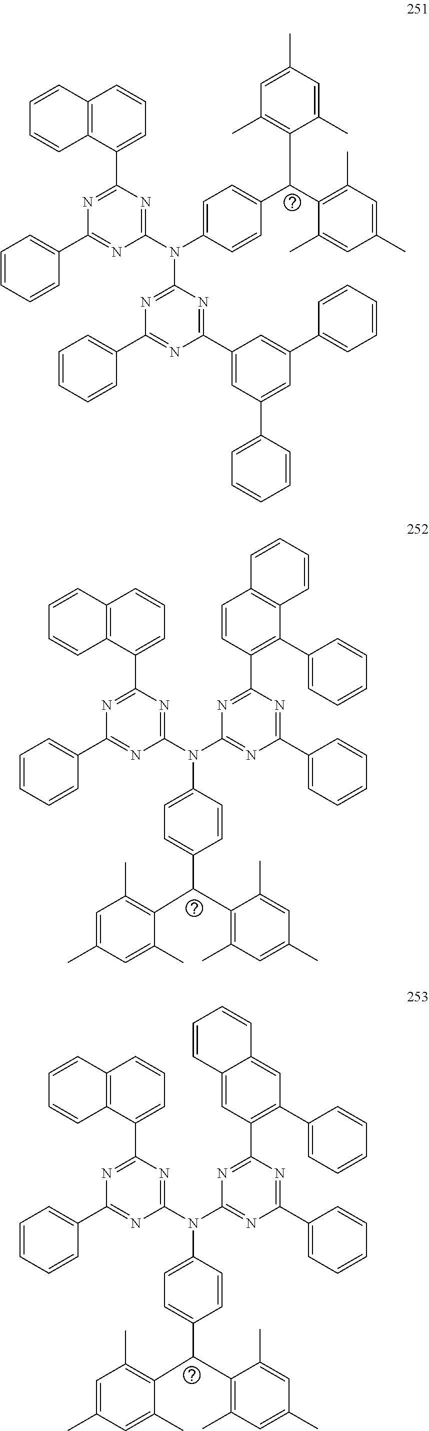 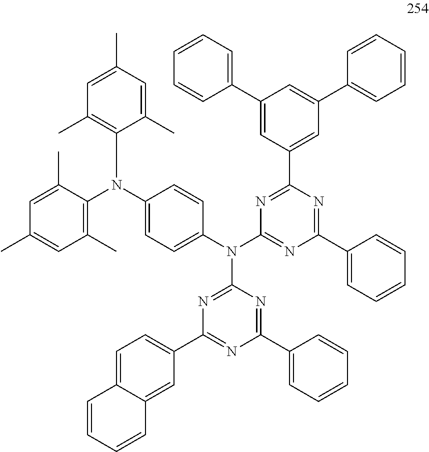 [SMILES: Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4)n3)cc2)c2c(C)cc(C)cc2C)c(C)c1] 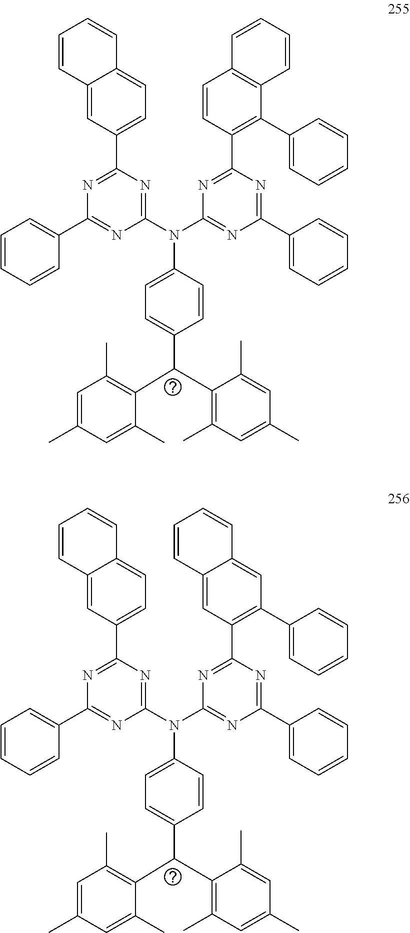 [SMILES: Cc1cc(C)c(C(c2ccc(N(c3nc(-c4ccccc4)nc(-c4ccc5ccccc5c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4ccc5ccccc5c4)n3)c3nc(-c4ccccc4)nc(-c4cc5ccccc5cc4-c4ccccc4)n3)cc2)c2c(C)cc(C)cc2C)c(C)c1] 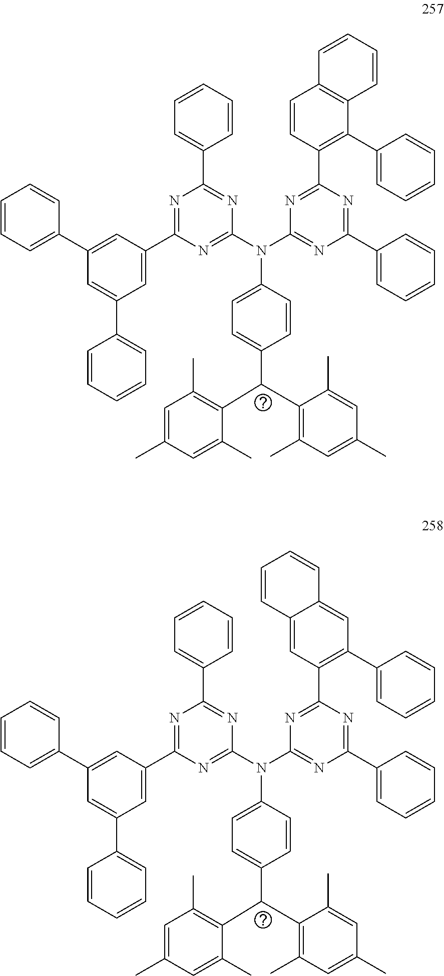 [SMILES: Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4-c4ccccc4)n3)cc2)c2c(C)cc(C)cc2C)c(C)c1 ; Cc1cc(C)c(C(c2ccc(N(c3nc(-c4ccccc4)nc(-c4cc(-c5ccccc5)cc(-c5ccccc5)c4)n3)c3nc(-c4ccccc4)nc(-c4cc5ccccc5cc4-c4ccccc4)n3)cc2)c2c(C)cc(C)cc2C)c(C)c1] 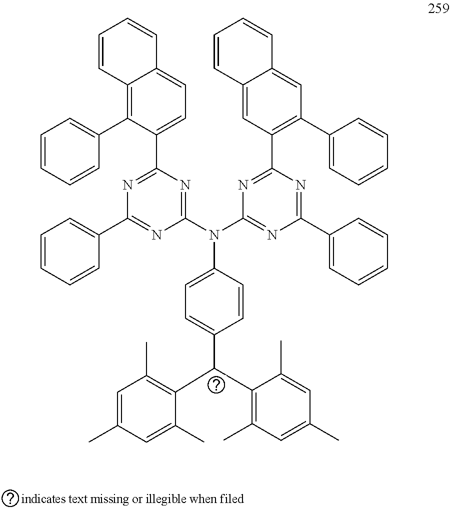 [SMILES: Cc1cc(C)c(C(c2ccc(N(c3nc(-c4ccccc4)nc(-c4cc5ccccc5cc4-c4ccccc4)n3)c3nc(-c4ccccc4)nc(-c4ccc5ccccc5c4-c4ccccc4)n3)cc2)c2c(C)cc(C)cc2C)c(C)c1] 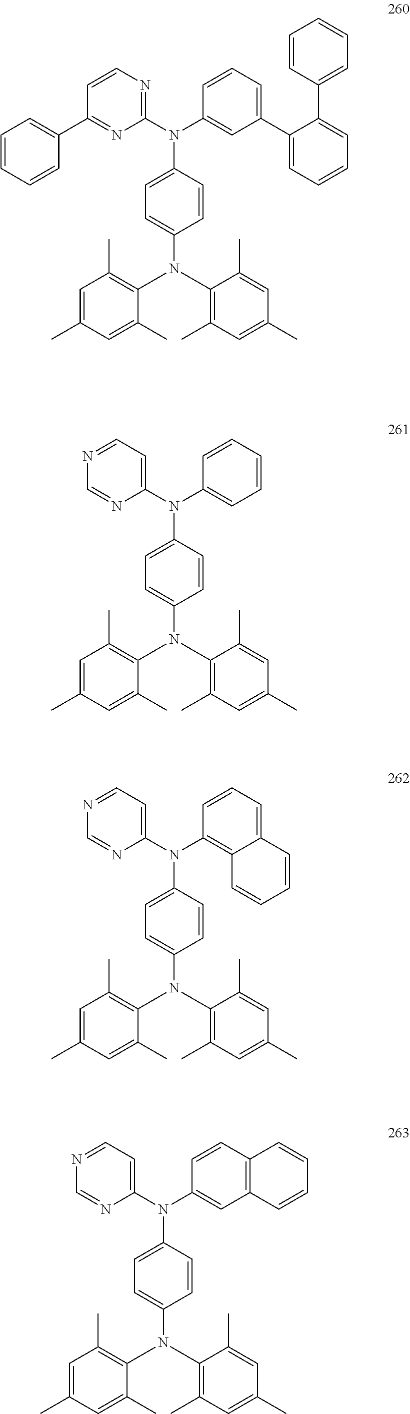 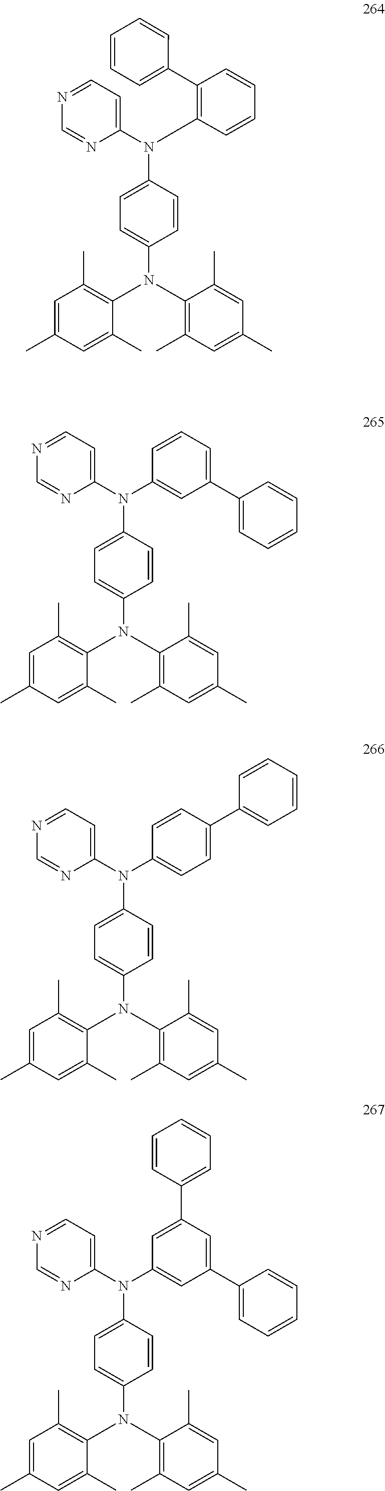 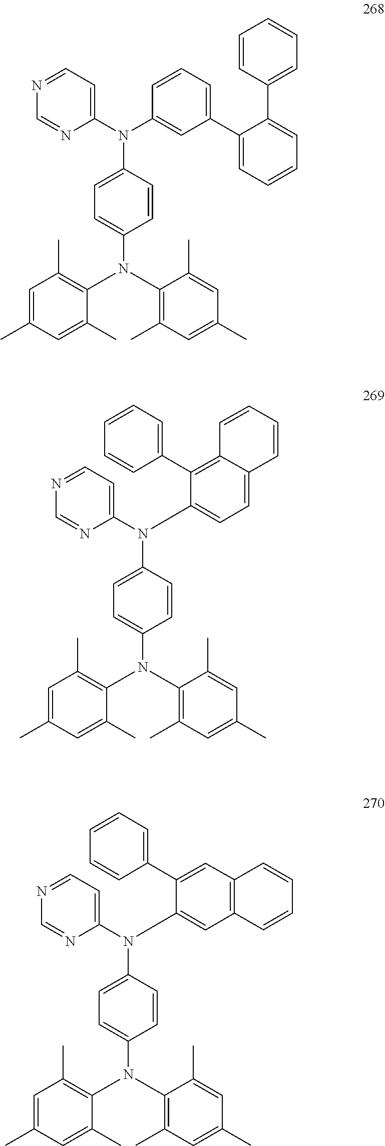 [SMILES: Cc1cc(C)c(N(c2ccc(N(c3cccc(-c4ccccc4-c4ccccc4)c3)c3ccncn3)cc2)c2c(C)cc(C)cc2C)c(C)c1 ; Cc1cc(C)c(N(c2ccc(N(c3ccncn3)c3ccc4ccccc4c3-c3ccccc3)cc2)c2c(C)cc(C)cc2C)c(C)c1 ; Cc1cc(C)c(N(c2ccc(N(c3ccncn3)c3cc4ccccc4cc3-c3ccccc3)cc2)c2c(C)cc(C)cc2C)c(C)c1] 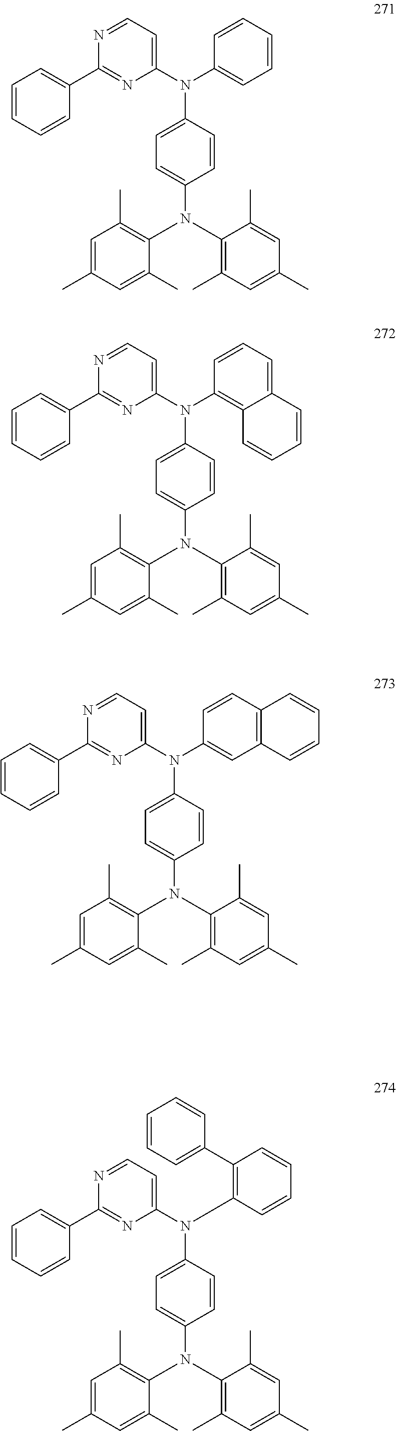 [SMILES: Cc1cc(C)c(N(c2ccc(N(c3ccccc3)c3ccnc(-c4ccccc4)n3)cc2)c2c(C)cc(C)cc2C)c(C)c1 ; Cc1cc(C)c(N(c2ccc(N(c3ccnc(-c4ccccc4)n3)c3cccc4ccccc34)cc2)c2c(C)cc(C)cc2C)c(C)c1 ; Cc1cc(C)c(N(c2ccc(N(c3ccc4ccccc4c3)c3ccnc(-c4ccccc4)n3)cc2)c2c(C)cc(C)cc2C)c(C)c1 ; Cc1cc(C)c(N(c2ccc(N(c3ccnc(-c4ccccc4)n3)c3ccccc3-c3ccccc3)cc2)c2c(C)cc(C)cc2C)c(C)c1] 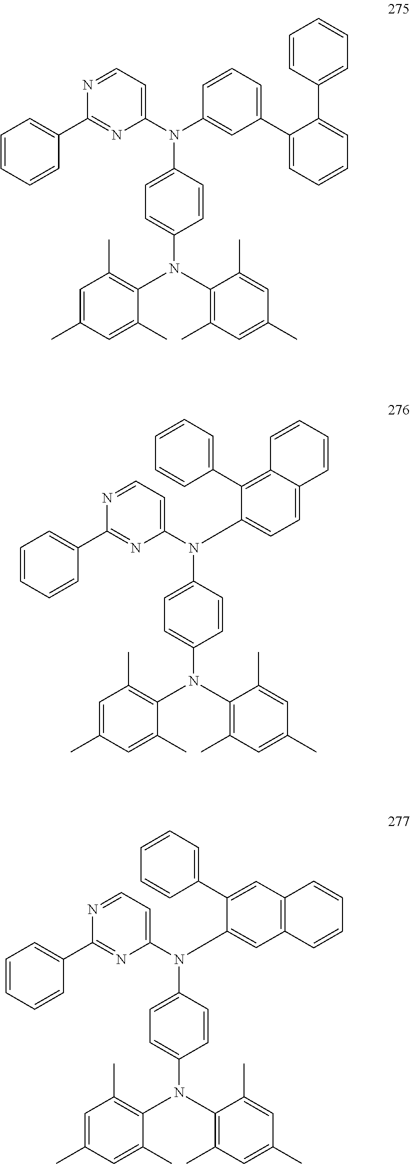 [SMILES: Cc1cc(C)c(N(c2ccc(N(c3cccc(-c4ccccc4-c4ccccc4)c3)c3ccnc(-c4ccccc4)n3)cc2)c2c(C)cc(C)cc2C)c(C)c1 ; Cc1cc(C)c(N(c2ccc(N(c3ccnc(-c4ccccc4)n3)c3ccc4ccccc4c3-c3ccccc3)cc2)c2c(C)cc(C)cc2C)c(C)c1 ; Cc1cc(C)c(N(c2ccc(N(c3ccnc(-c4ccccc4)n3)c3cc4ccccc4cc3-c3ccccc3)cc2)c2c(C)cc(C)cc2C)c(C)c1] 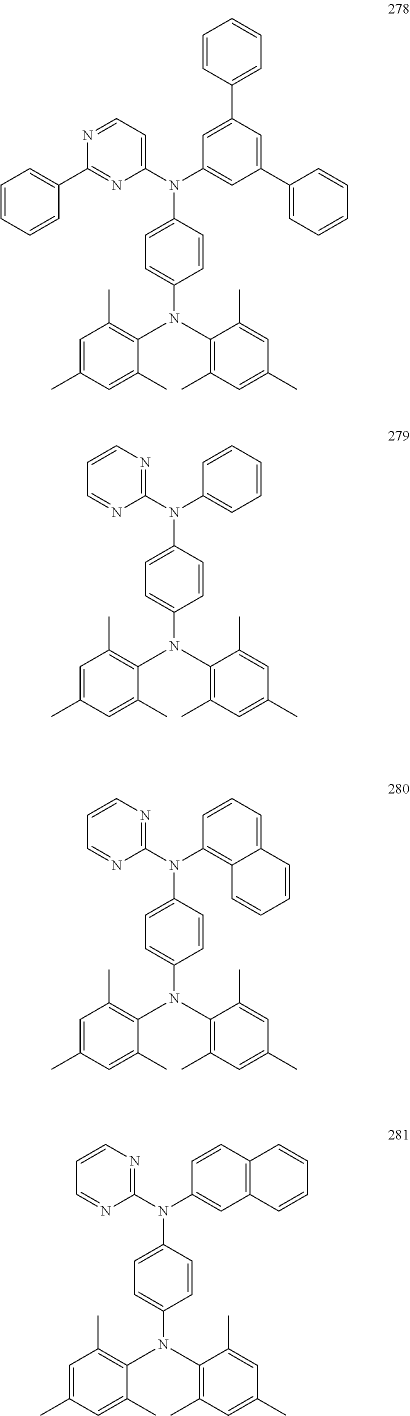 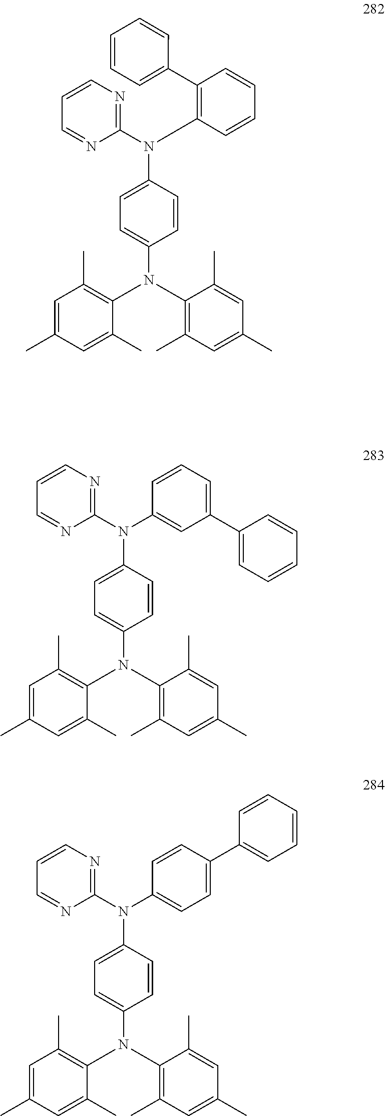 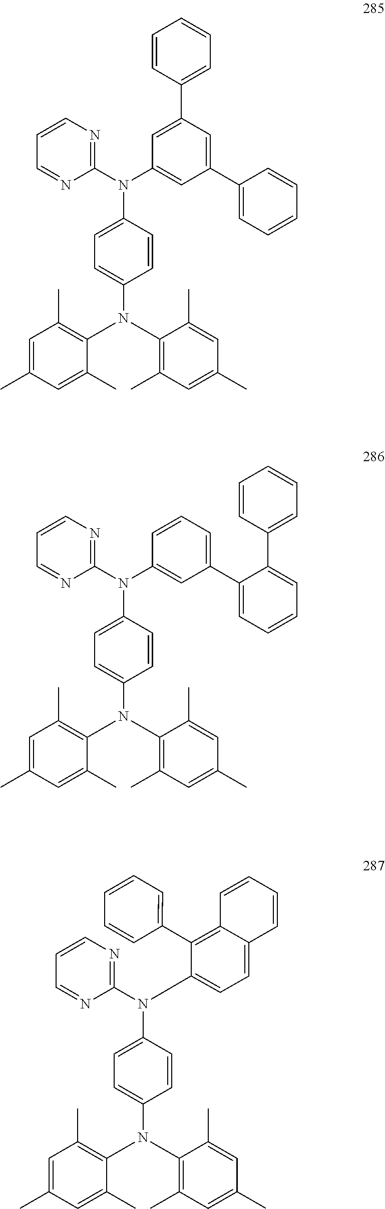 [SMILES: Cc1cc(C)c(N(c2ccc(N(c3cc(-c4ccccc4)cc(-c4ccccc4)c3)c3ncccn3)cc2)c2c(C)cc(C)cc2C)c(C)c1 ; Cc1cc(C)c(N(c2ccc(N(c3cccc(-c4ccccc4-c4ccccc4)c3)c3ncccn3)cc2)c2c(C)cc(C)cc2C)c(C)c1 ; Cc1cc(C)c(N(c2ccc(N(c3ncccn3)c3ccc4ccccc4c3-c3ccccc3)cc2)c2c(C)cc(C)cc2C)c(C)c1] 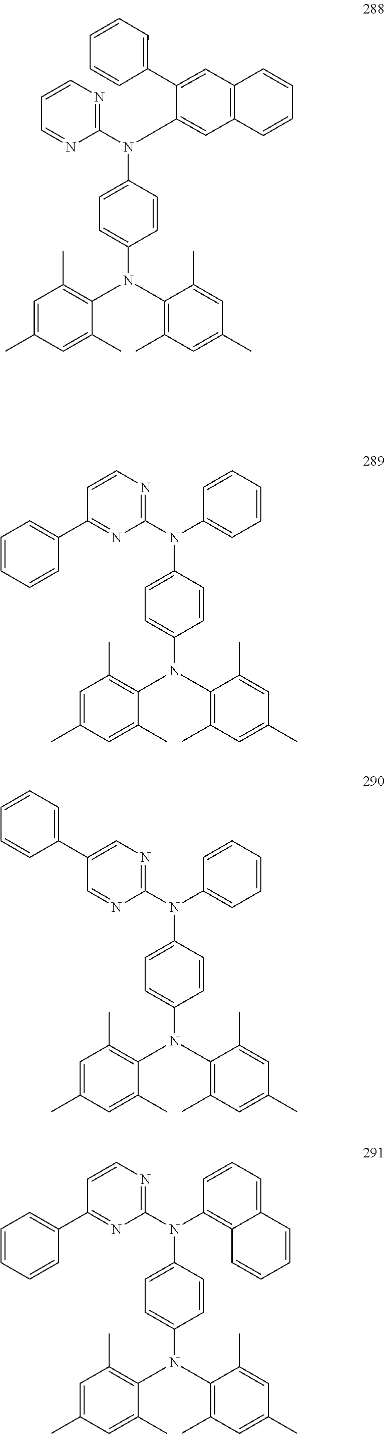 [SMILES: Cc1cc(C)c(N(c2ccc(N(c3ncccn3)c3cc4ccccc4cc3-c3ccccc3)cc2)c2c(C)cc(C)cc2C)c(C)c1 ; Cc1cc(C)c(N(c2ccc(N(c3ccccc3)c3nccc(-c4ccccc4)n3)cc2)c2c(C)cc(C)cc2C)c(C)c1 ; Cc1cc(C)c(N(c2ccc(N(c3ccccc3)c3ncc(-c4ccccc4)cn3)cc2)c2c(C)cc(C)cc2C)c(C)c1 ; Cc1cc(C)c(N(c2ccc(N(c3nccc(-c4ccccc4)n3)c3cccc4ccccc34)cc2)c2c(C)cc(C)cc2C)c(C)c1] 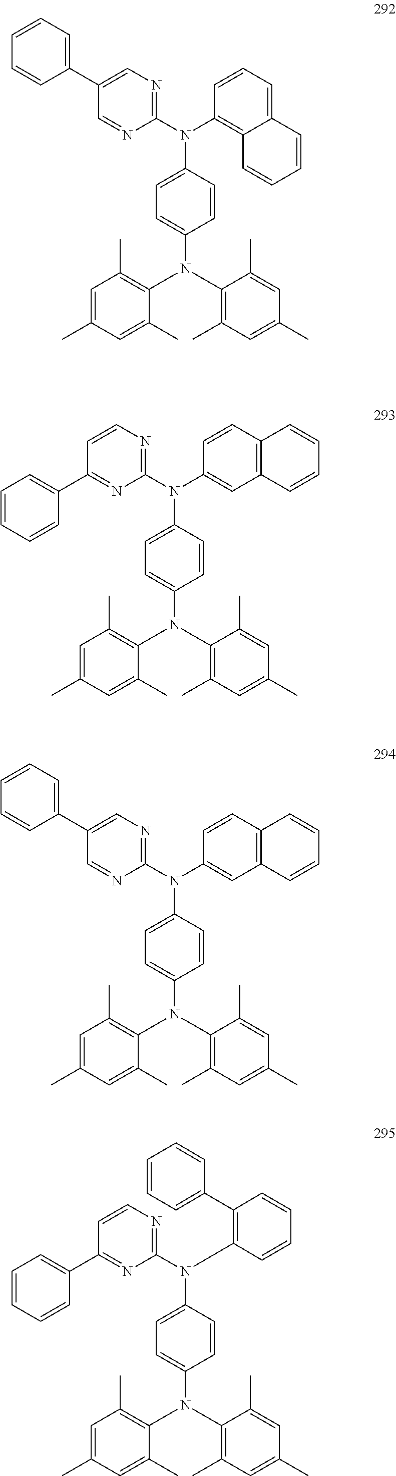 [SMILES: Cc1cc(C)c(N(c2ccc(N(c3ncc(-c4ccccc4)cn3)c3cccc4ccccc34)cc2)c2c(C)cc(C)cc2C)c(C)c1 ; Cc1cc(C)c(N(c2ccc(N(c3ccc4ccccc4c3)c3nccc(-c4ccccc4)n3)cc2)c2c(C)cc(C)cc2C)c(C)c1 ; Cc1cc(C)c(N(c2ccc(N(c3ccc4ccccc4c3)c3ncc(-c4ccccc4)cn3)cc2)c2c(C)cc(C)cc2C)c(C)c1 ; Cc1cc(C)c(N(c2ccc(N(c3nccc(-c4ccccc4)n3)c3ccccc3-c3ccccc3)cc2)c2c(C)cc(C)cc2C)c(C)c1] 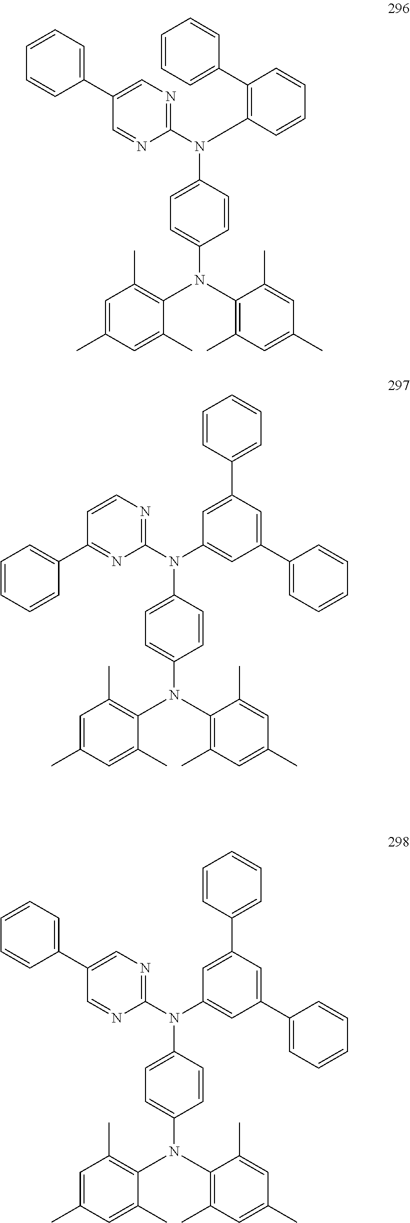 [SMILES: Cc1cc(C)c(N(c2ccc(N(c3ncc(-c4ccccc4)cn3)c3ccccc3-c3ccccc3)cc2)c2c(C)cc(C)cc2C)c(C)c1 ; Cc1cc(C)c(N(c2ccc(N(c3cc(-c4ccccc4)cc(-c4ccccc4)c3)c3nccc(-c4ccccc4)n3)cc2)c2c(C)cc(C)cc2C)c(C)c1 ; Cc1cc(C)c(N(c2ccc(N(c3cc(-c4ccccc4)cc(-c4ccccc4)c3)c3ncc(-c4ccccc4)cn3)cc2)c2c(C)cc(C)cc2C)c(C)c1] 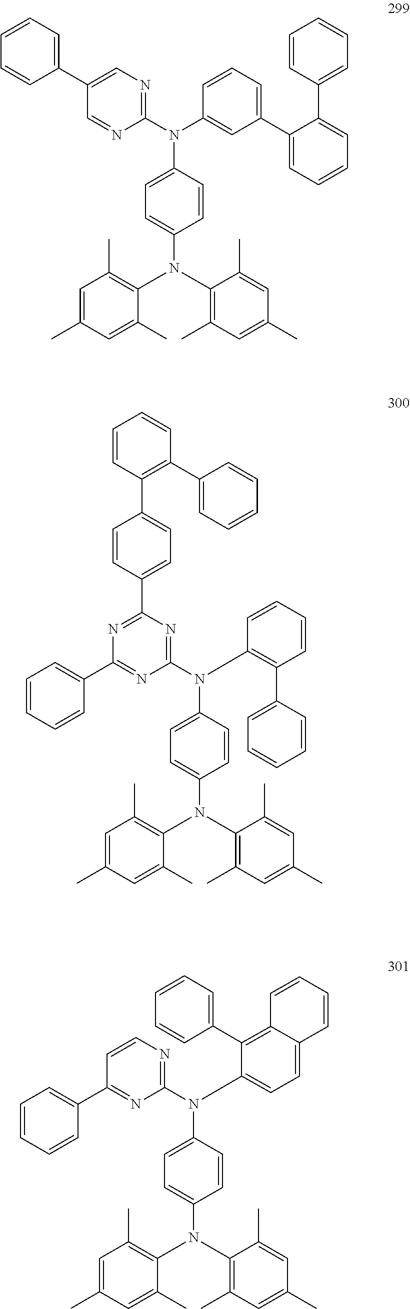 [SMILES: Cc1cc(C)c(N(c2ccc(N(c3cccc(-c4ccccc4-c4ccccc4)c3)c3ncc(-c4ccccc4)cn3)cc2)c2c(C)cc(C)cc2C)c(C)c1 ; Cc1cc(C)c(N(c2ccc(N(c3nc(-c4ccccc4)nc(-c4ccc(-c5ccccc5-c5ccccc5)cc4)n3)c3ccccc3-c3ccccc3)cc2)c2c(C)cc(C)cc2C)c(C)c1 ; Cc1cc(C)c(N(c2ccc(N(c3nccc(-c4ccccc4)n3)c3ccc4ccccc4c3-c3ccccc3)cc2)c2c(C)cc(C)cc2C)c(C)c1] 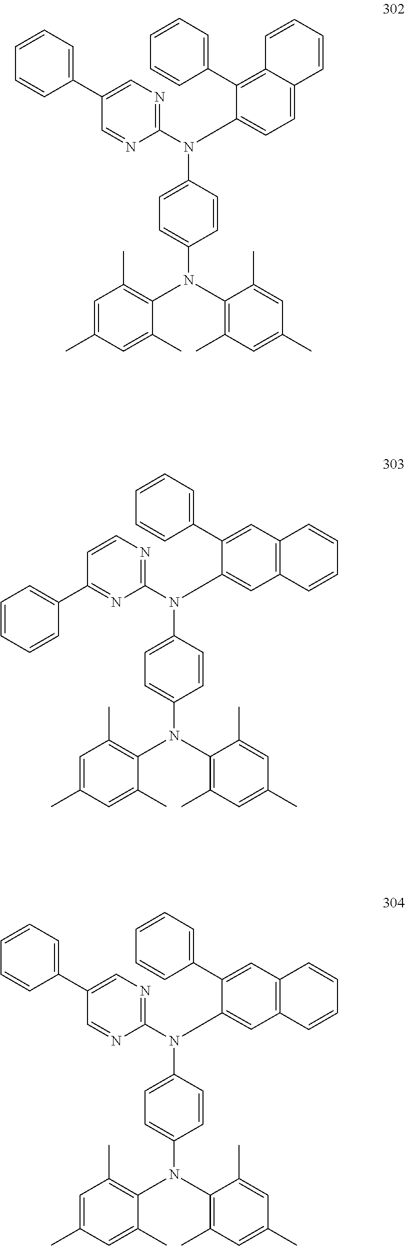 [SMILES: Cc1cc(C)c(N(c2ccc(N(c3ncc(-c4ccccc4)cn3)c3ccc4ccccc4c3-c3ccccc3)cc2)c2c(C)cc(C)cc2C)c(C)c1 ; Cc1cc(C)c(N(c2ccc(N(c3nccc(-c4ccccc4)n3)c3cc4ccccc4cc3-c3ccccc3)cc2)c2c(C)cc(C)cc2C)c(C)c1 ; Cc1cc(C)c(N(c2ccc(N(c3ncc(-c4ccccc4)cn3)c3cc4ccccc4cc3-c3ccccc3)cc2)c2c(C)cc(C)cc2C)c(C)c1] 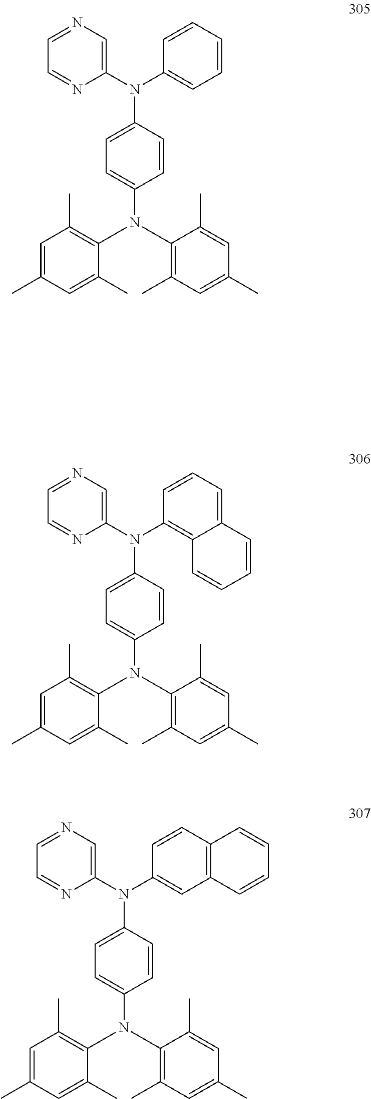 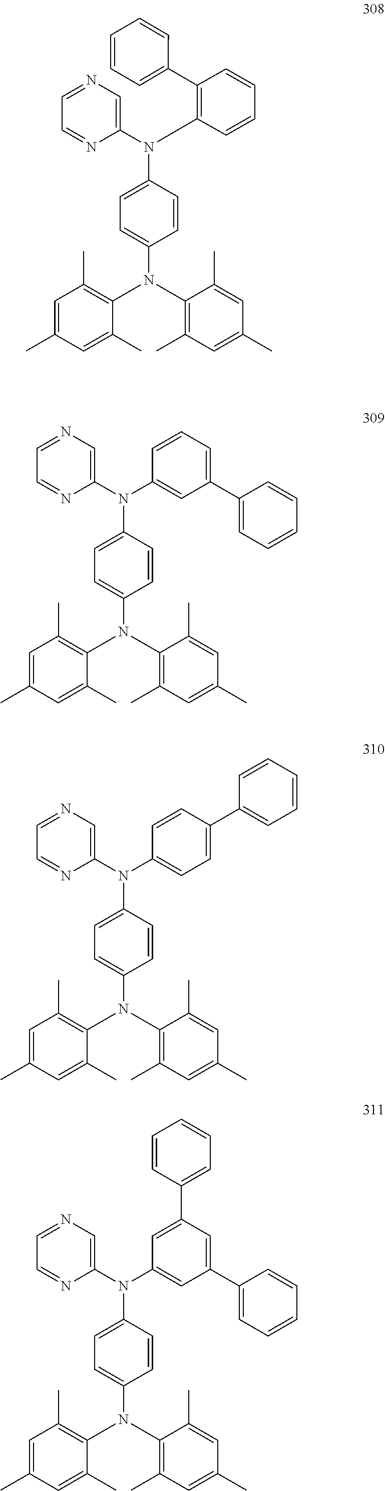 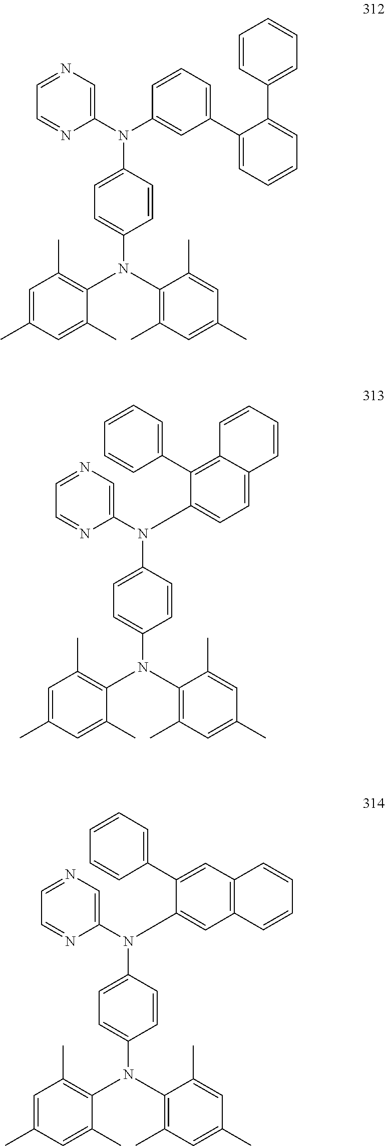 [SMILES: Cc1cc(C)c(N(c2ccc(N(c3cccc(-c4ccccc4-c4ccccc4)c3)c3cnccn3)cc2)c2c(C)cc(C)cc2C)c(C)c1 ; Cc1cc(C)c(N(c2ccc(N(c3cnccn3)c3ccc4ccccc4c3-c3ccccc3)cc2)c2c(C)cc(C)cc2C)c(C)c1 ; Cc1cc(C)c(N(c2ccc(N(c3cnccn3)c3cc4ccccc4cc3-c3ccccc3)cc2)c2c(C)cc(C)cc2C)c(C)c1] 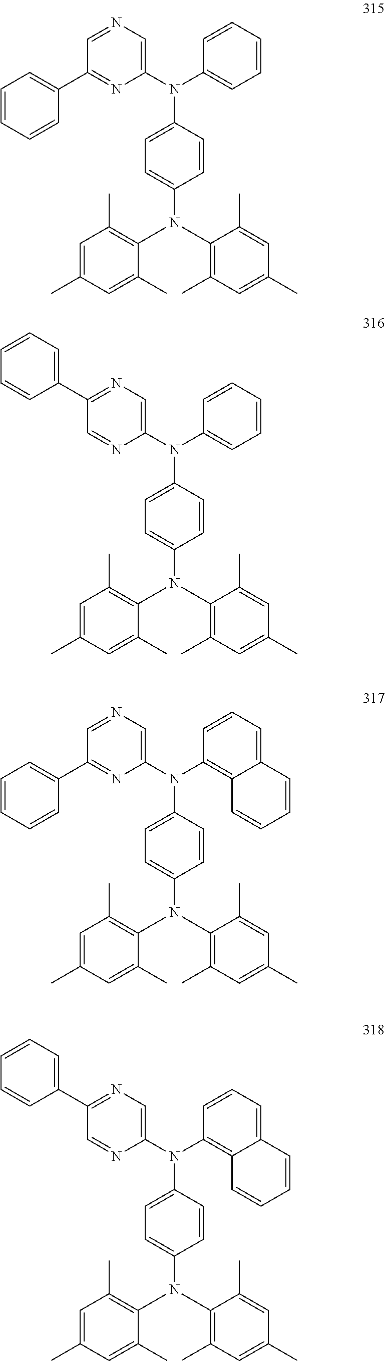 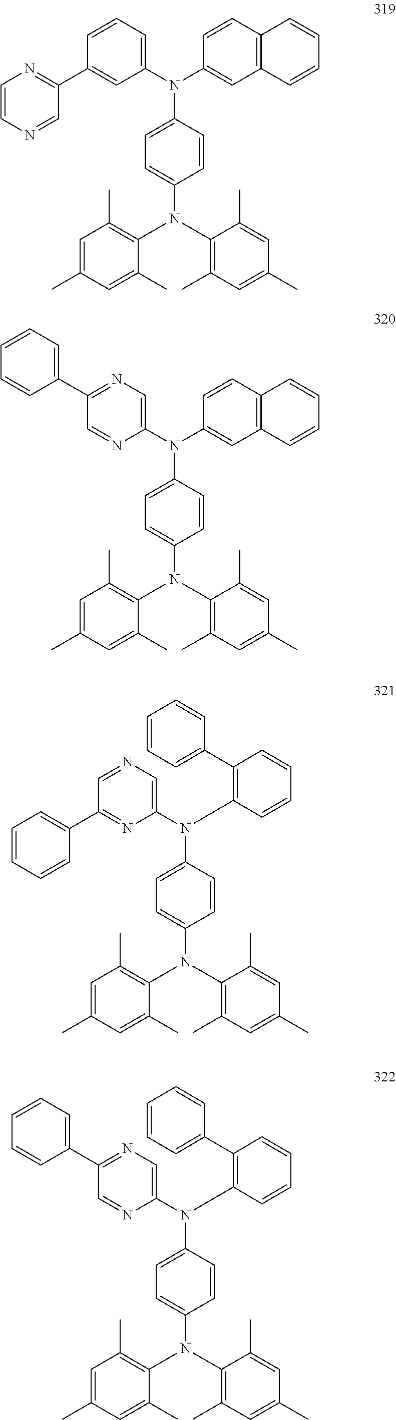 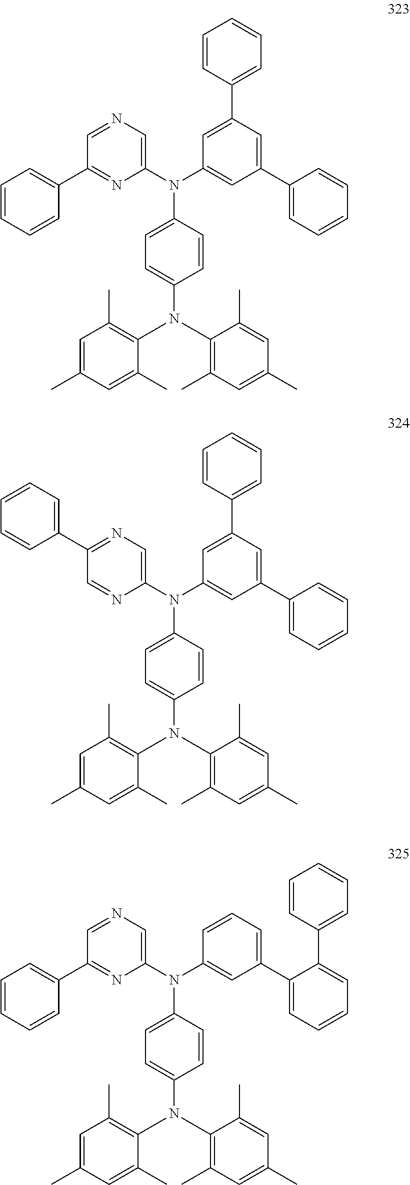 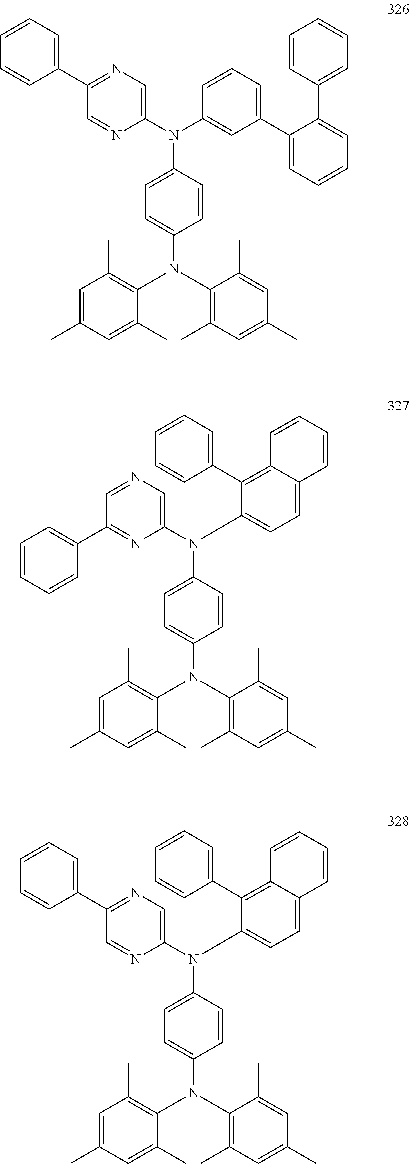 [SMILES: Cc1cc(C)c(N(c2ccc(N(c3cccc(-c4ccccc4-c4ccccc4)c3)c3cnc(-c4ccccc4)cn3)cc2)c2c(C)cc(C)cc2C)c(C)c1 ; Cc1cc(C)c(N(c2ccc(N(c3cncc(-c4ccccc4)n3)c3ccc4ccccc4c3-c3ccccc3)cc2)c2c(C)cc(C)cc2C)c(C)c1 ; Cc1cc(C)c(N(c2ccc(N(c3cnc(-c4ccccc4)cn3)c3ccc4ccccc4c3-c3ccccc3)cc2)c2c(C)cc(C)cc2C)c(C)c1] 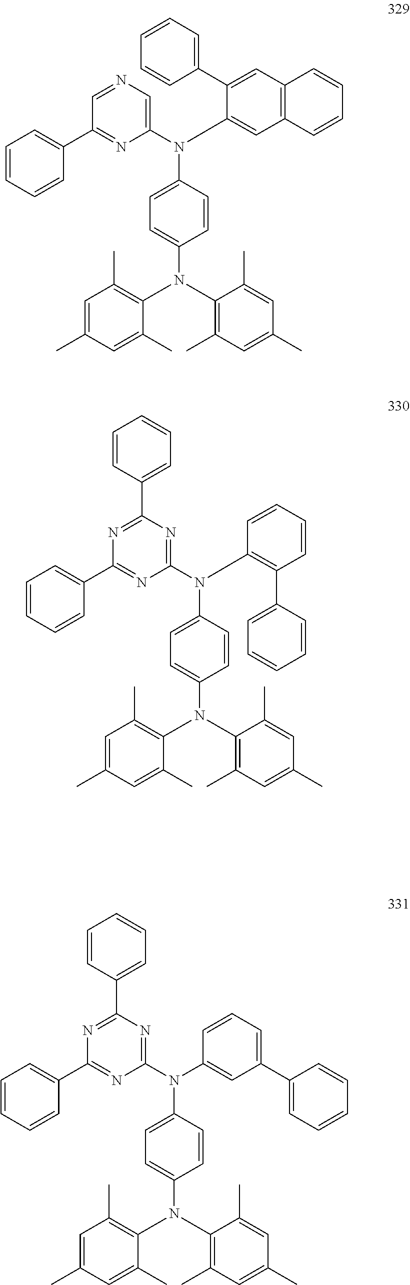 [SMILES: Cc1cc(C)c(N(c2ccc(N(c3cncc(-c4ccccc4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4ccccc4)n3)c3ccccc3-c3ccccc3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4ccccc4)n3)cc2)c2c(C)cc(C)cc2C)c(C)c1] 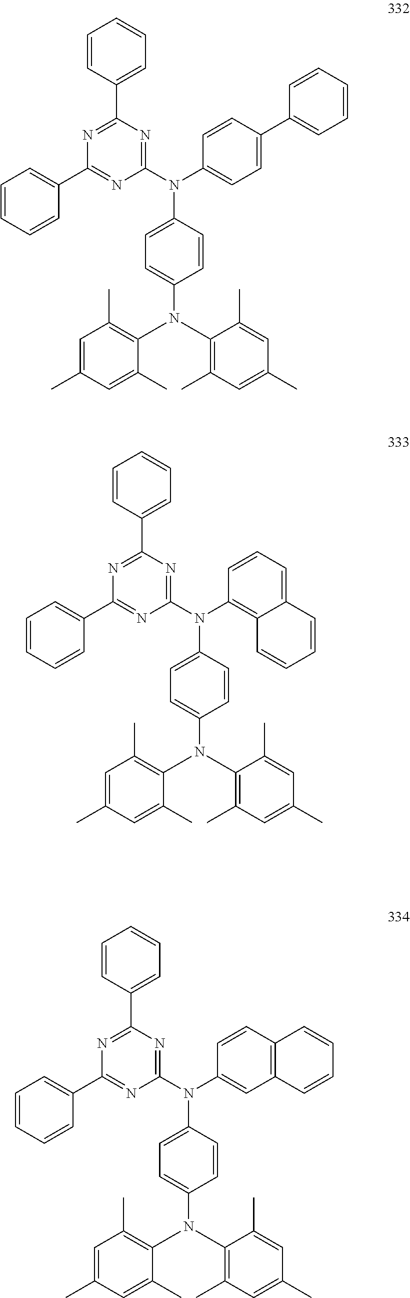 [SMILES: Cc1cc(C)c(N(c2ccc(N(c3ccc(-c4ccccc4)cc3)c3nc(-c4ccccc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3cccc4ccccc34)cc2)c2c(C)cc(C)cc2C)c(C)c1 ; Cc1cc(C)c(N(c2ccc(N(c3ccc4ccccc4c3)c3nc(-c4ccccc4)nc(-c4ccccc4)n3)cc2)c2c(C)cc(C)cc2C)c(C)c1] 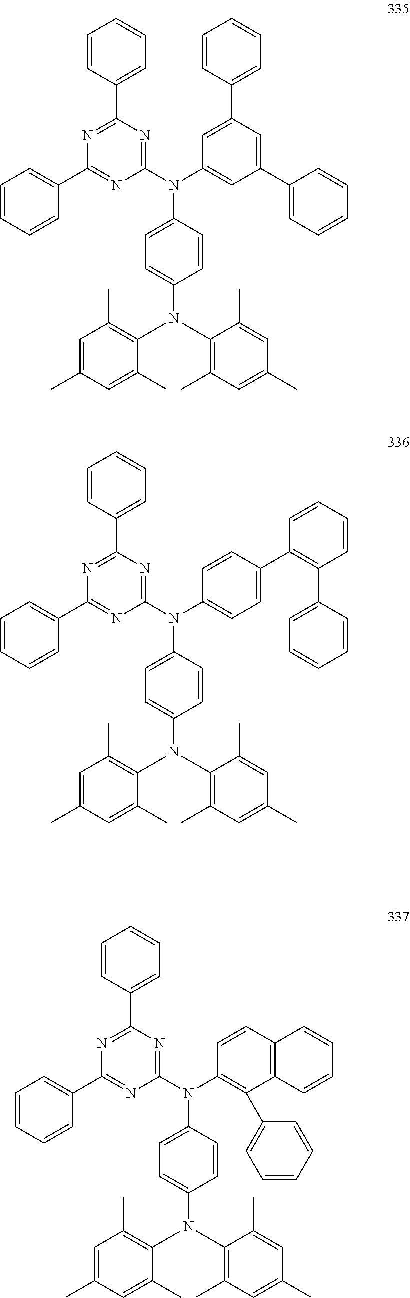 [SMILES: Cc1cc(C)c(N(c2ccc(N(c3cc(-c4ccccc4)cc(-c4ccccc4)c3)c3nc(-c4ccccc4)nc(-c4ccccc4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3ccc4ccccc4c3-c3ccccc3)cc2)c2c(C)cc(C)cc2C)c(C)c1] 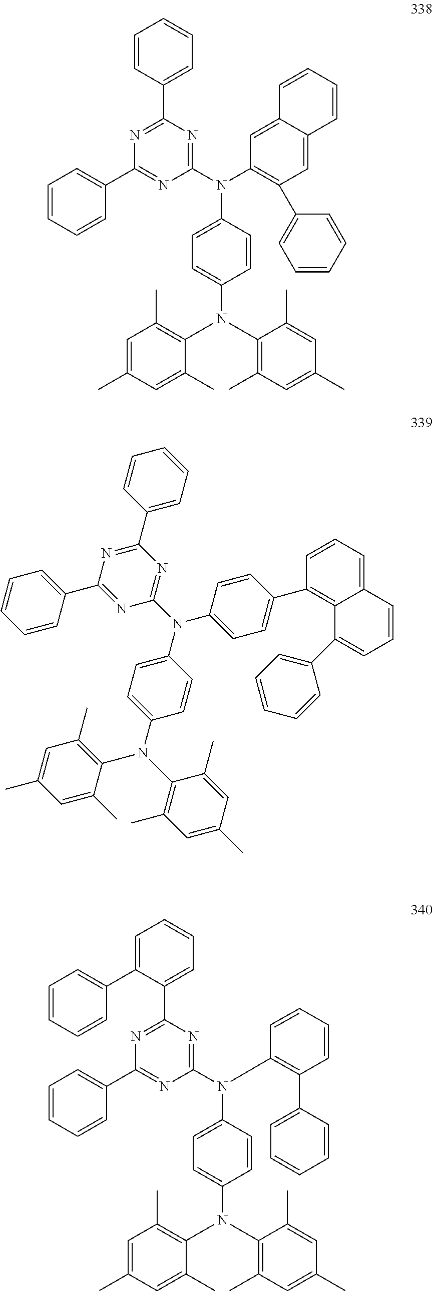 [SMILES: Cc1cc(C)c(N(c2ccc(N(c3nc(-c4ccccc4)nc(-c4ccccc4)n3)c3cc4ccccc4cc3-c3ccccc3)cc2)c2c(C)cc(C)cc2C)c(C)c1 ; Cc1cc(C)c(N(c2ccc(N(c3ccc(-c4cccc5cccc(-c6ccccc6)c45)cc3)c3nc(-c4ccccc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4-c4ccccc4)n3)c3ccccc3-c3ccccc3)cc2)c2c(C)cc(C)cc2C)c(C)c1] 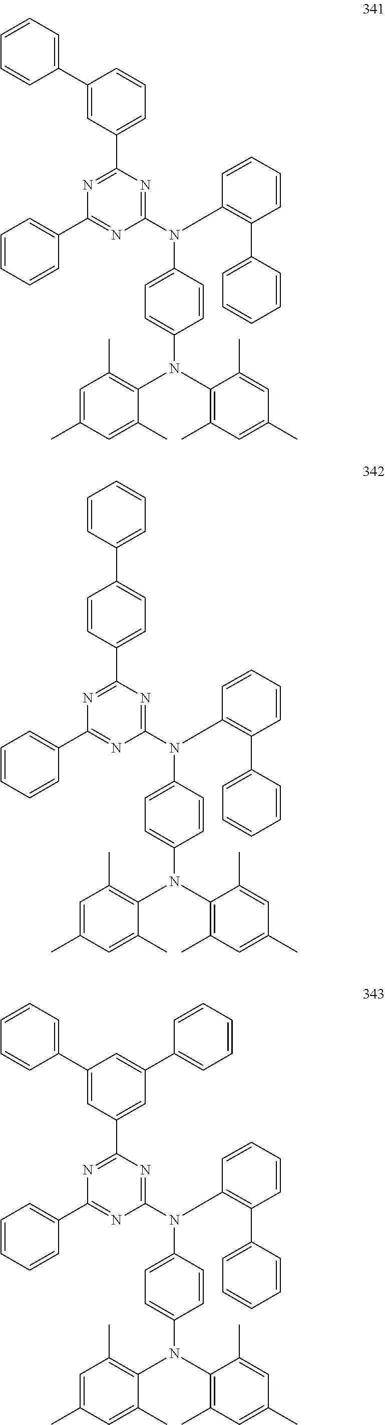 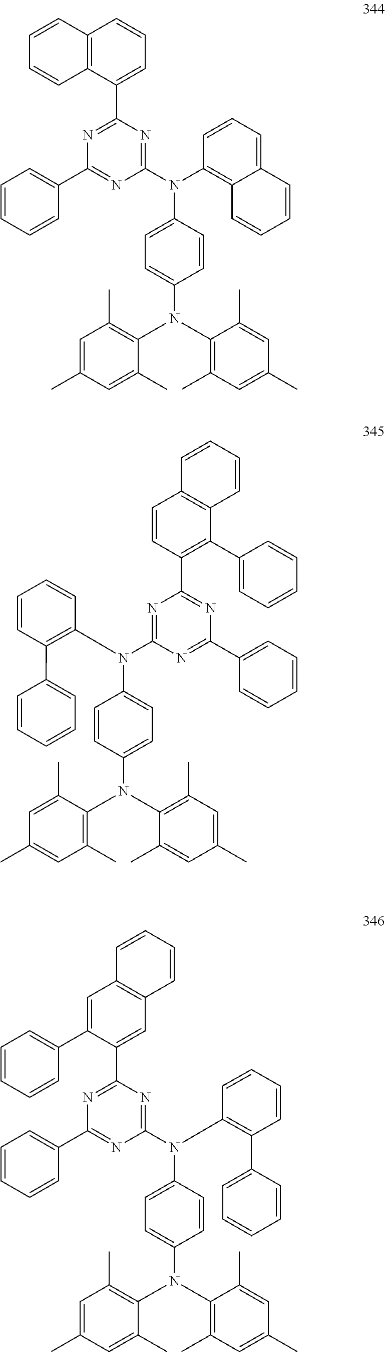 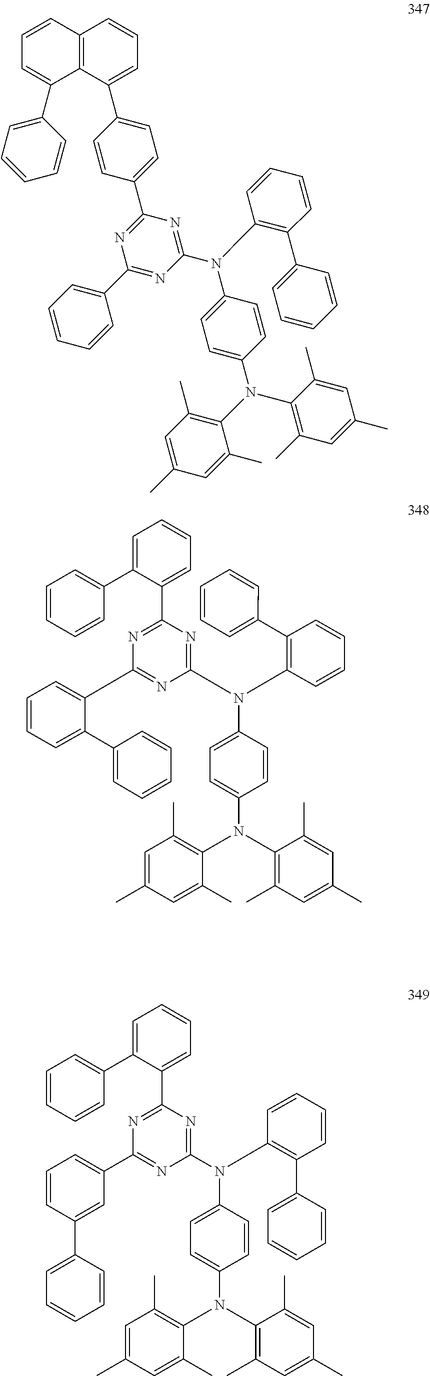 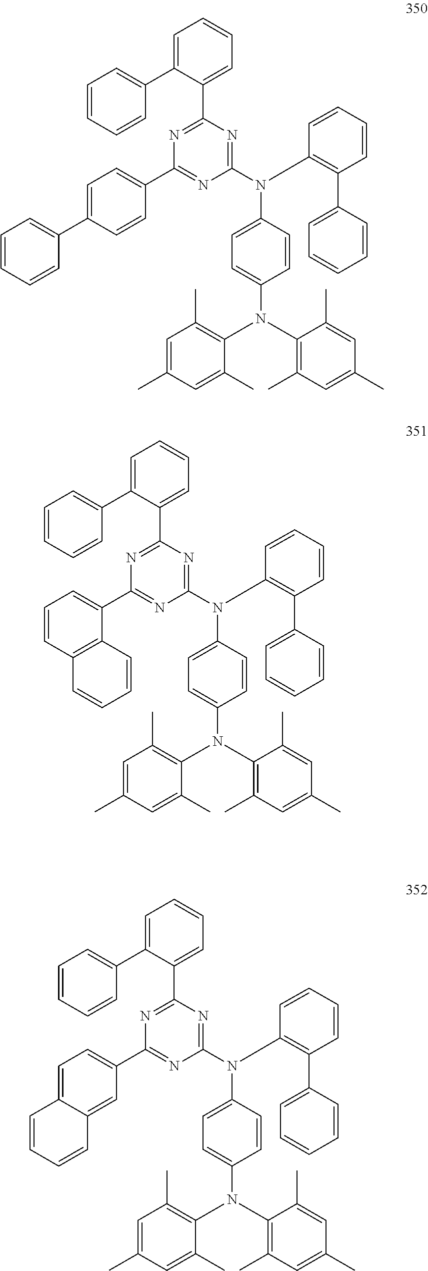 [SMILES: Cc1cc(C)c(N(c2ccc(N(c3nc(-c4ccc(-c5ccccc5)cc4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4ccccc4-c4ccccc4)nc(-c4cccc5ccccc45)n3)c3ccccc3-c3ccccc3)cc2)c2c(C)cc(C)cc2C)c(C)c1 ; Cc1cc(C)c(N(c2ccc(N(c3nc(-c4ccc5ccccc5c4)nc(-c4ccccc4-c4ccccc4)n3)c3ccccc3-c3ccccc3)cc2)c2c(C)cc(C)cc2C)c(C)c1] 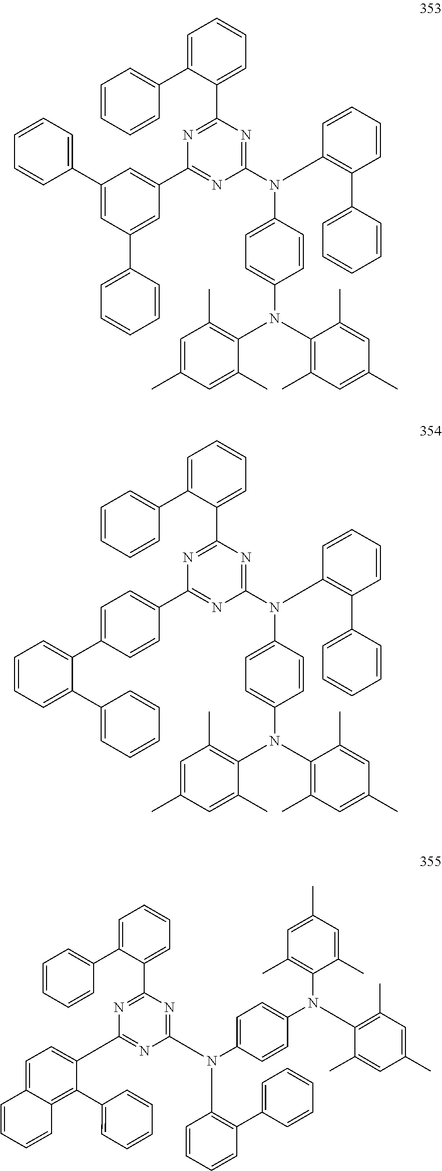 [SMILES: Cc1cc(C)c(N(c2ccc(N(c3nc(-c4cc(-c5ccccc5)cc(-c5ccccc5)c4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4ccc(-c5ccccc5-c5ccccc5)cc4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4ccccc4-c4ccccc4)nc(-c4ccc5ccccc5c4-c4ccccc4)n3)c3ccccc3-c3ccccc3)cc2)c2c(C)cc(C)cc2C)c(C)c1] 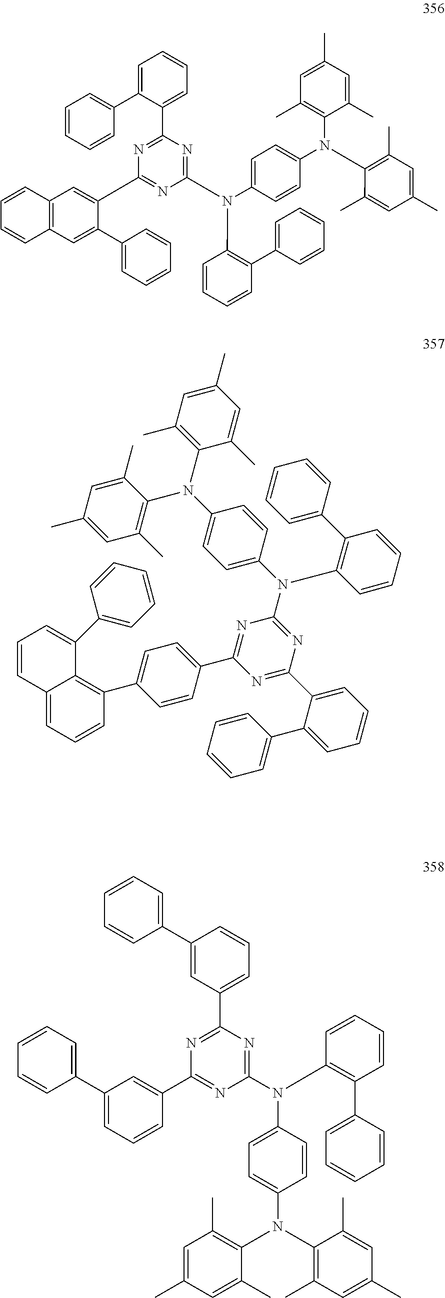 [SMILES: Cc1cc(C)c(N(c2ccc(N(c3nc(-c4ccccc4-c4ccccc4)nc(-c4cc5ccccc5cc4-c4ccccc4)n3)c3ccccc3-c3ccccc3)cc2)c2c(C)cc(C)cc2C)c(C)c1 ; Cc1cc(C)c(N(c2ccc(N(c3nc(-c4ccc(-c5cccc6cccc(-c7ccccc7)c56)cc4)nc(-c4ccccc4-c4ccccc4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cccc(-c5ccccc5)c4)n3)c3ccccc3-c3ccccc3)cc2)c2c(C)cc(C)cc2C)c(C)c1] 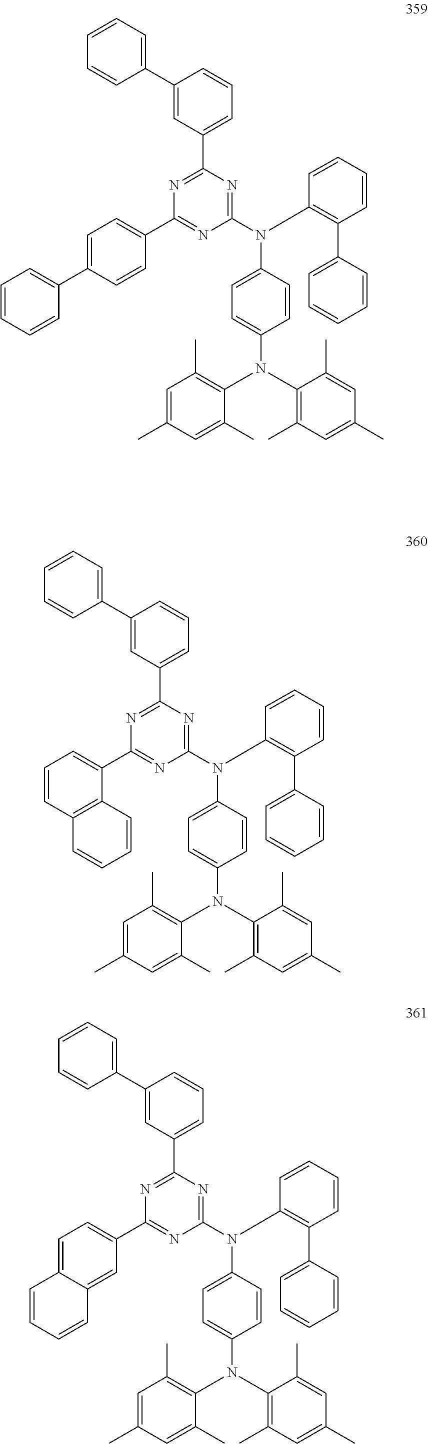 [SMILES: Cc1cc(C)c(N(c2ccc(N(c3nc(-c4ccc(-c5ccccc5)cc4)nc(-c4cccc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cccc5ccccc45)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4ccc5ccccc5c4)n3)c3ccccc3-c3ccccc3)cc2)c2c(C)cc(C)cc2C)c(C)c1] 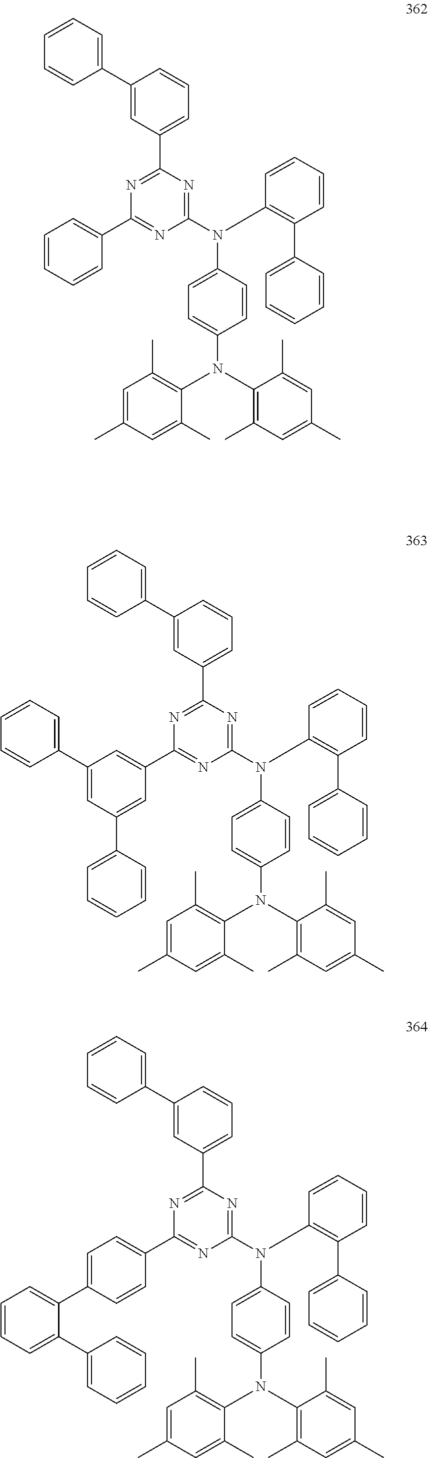 [SMILES: Cc1cc(C)c(N(c2ccc(N(c3nc(-c4ccccc4)nc(-c4cccc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cc(-c5ccccc5)cc(-c5ccccc5)c4)n3)c3ccccc3-c3ccccc3)cc2)c2c(C)cc(C)cc2C)c(C)c1 ; Cc1cc(C)c(N(c2ccc(N(c3nc(-c4ccc(-c5ccccc5-c5ccccc5)cc4)nc(-c4cccc(-c5ccccc5)c4)n3)c3ccccc3-c3ccccc3)cc2)c2c(C)cc(C)cc2C)c(C)c1] 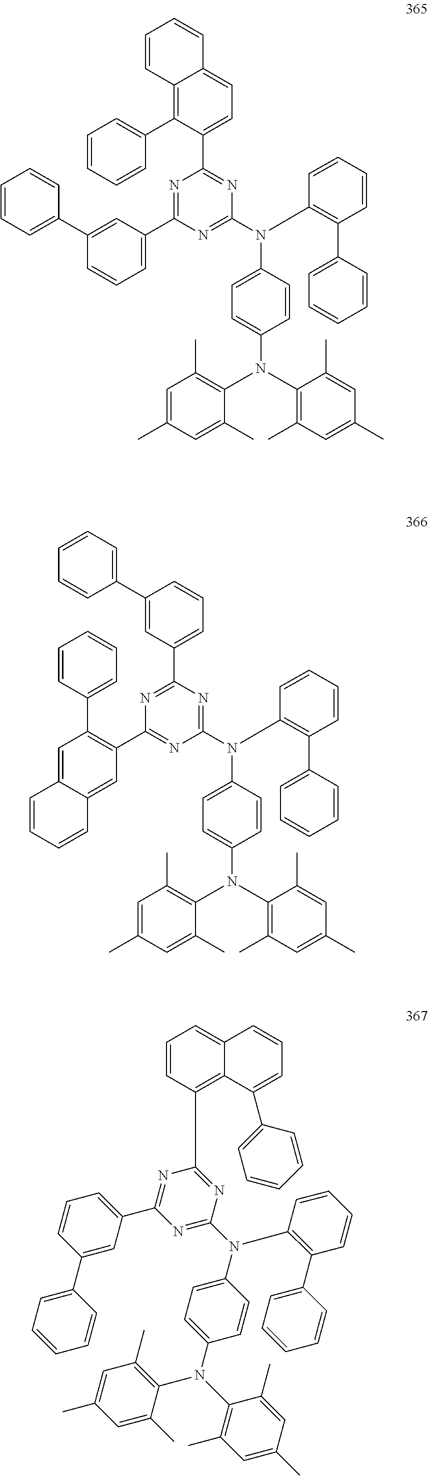 [SMILES: Cc1cc(C)c(N(c2ccc(N(c3nc(-c4cccc(-c5ccccc5)c4)nc(-c4ccc5ccccc5c4-c4ccccc4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cc5ccccc5cc4-c4ccccc4)n3)c3ccccc3-c3ccccc3)cc2)c2c(C)cc(C)cc2C)c(C)c1 ; Cc1cc(C)c(N(c2ccc(N(c3nc(-c4cccc(-c5ccccc5)c4)nc(-c4cccc5cccc(-c6ccccc6)c45)n3)c3ccccc3-c3ccccc3)cc2)c2c(C)cc(C)cc2C)c(C)c1] 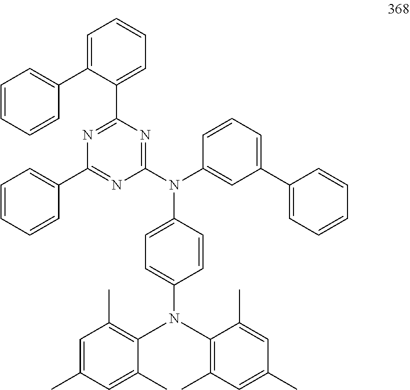 [SMILES: Cc1cc(C)c(N(c2ccc(N(c3cccc(-c4ccccc4)c3)c3nc(-c4ccccc4)nc(-c4ccccc4-c4ccccc4)n3)cc2)c2c(C)cc(C)cc2C)c(C)c1] 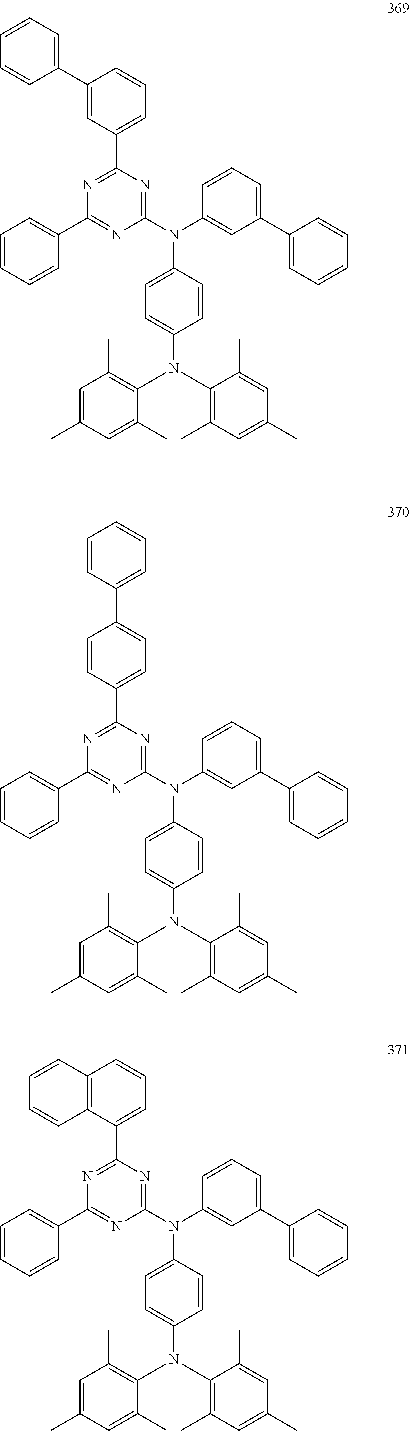 [SMILES: Cc1cc(C)c(N(c2ccc(N(c3cccc(-c4ccccc4)c3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4ccc(-c5ccccc5)cc4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4cccc5ccccc45)n3)cc2)c2c(C)cc(C)cc2C)c(C)c1] 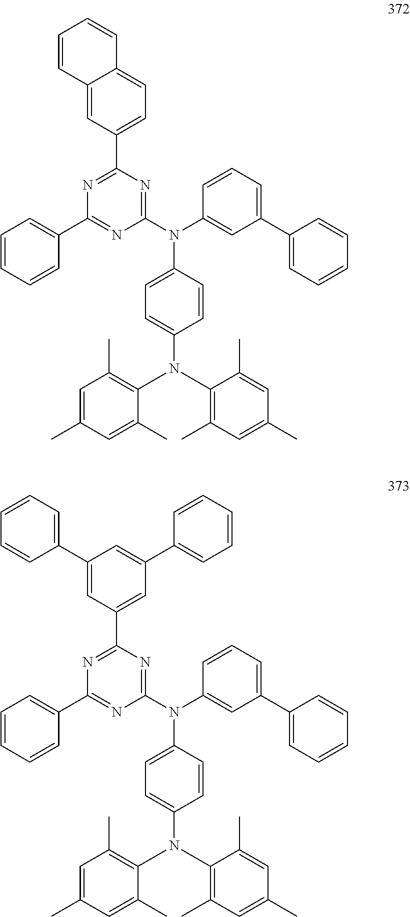 [SMILES: Cc1cc(C)c(N(c2ccc(N(c3cccc(-c4ccccc4)c3)c3nc(-c4ccccc4)nc(-c4ccc5ccccc5c4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4cc(-c5ccccc5)cc(-c5ccccc5)c4)n3)cc2)c2c(C)cc(C)cc2C)c(C)c1] 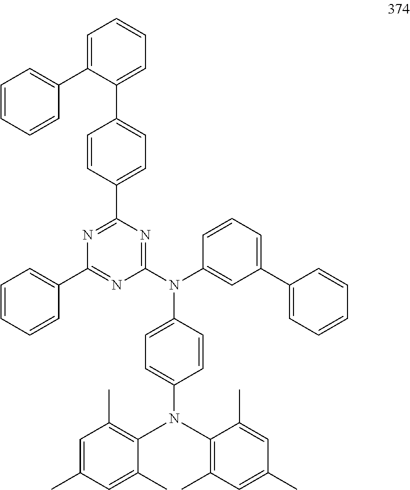 [SMILES: Cc1cc(C)c(N(c2ccc(N(c3cccc(-c4ccccc4)c3)c3nc(-c4ccccc4)nc(-c4ccc(-c5ccccc5-c5ccccc5)cc4)n3)cc2)c2c(C)cc(C)cc2C)c(C)c1] 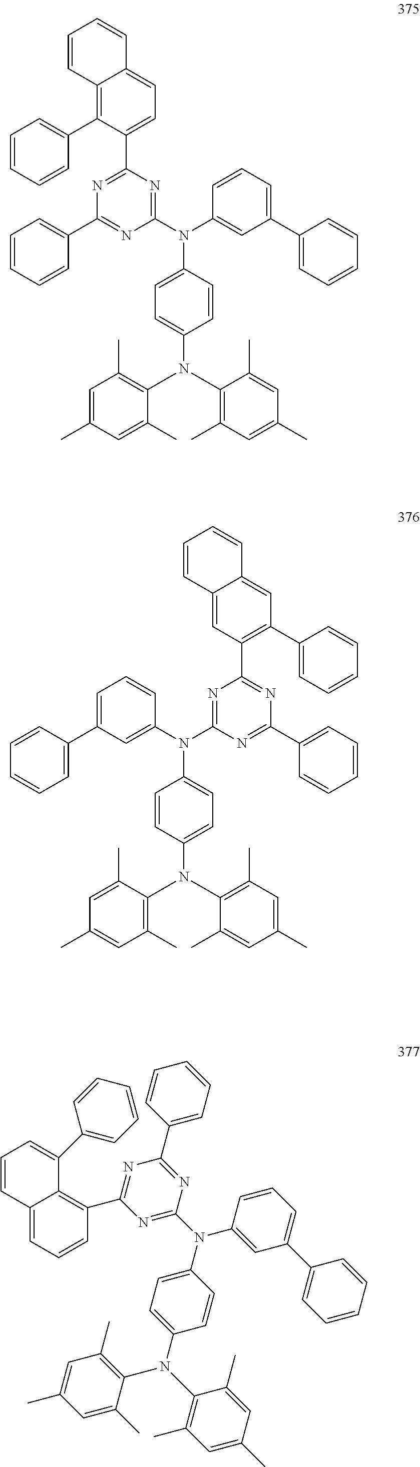 [SMILES: Cc1cc(C)c(N(c2ccc(N(c3cccc(-c4ccccc4)c3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3cccc(-c4ccccc4)c3)c3nc(-c4ccccc4)nc(-c4cccc5cccc(-c6ccccc6)c45)n3)cc2)c2c(C)cc(C)cc2C)c(C)c1] 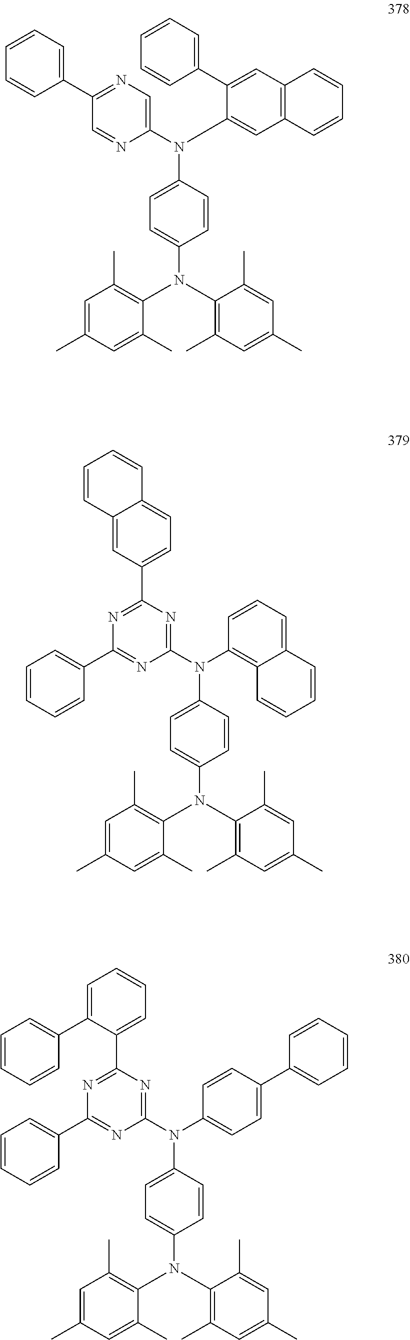 [SMILES: Cc1cc(C)c(N(c2ccc(N(c3cnc(-c4ccccc4)cn3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4ccc5ccccc5c4)n3)c3cccc4ccccc34)cc2)c2c(C)cc(C)cc2C)c(C)c1 ; Cc1cc(C)c(N(c2ccc(N(c3ccc(-c4ccccc4)cc3)c3nc(-c4ccccc4)nc(-c4ccccc4-c4ccccc4)n3)cc2)c2c(C)cc(C)cc2C)c(C)c1] 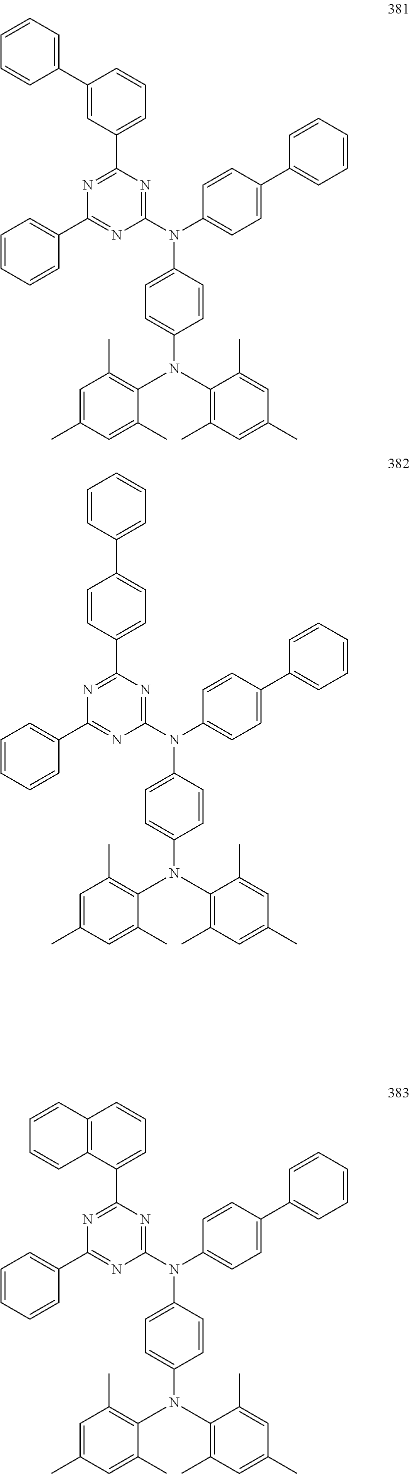 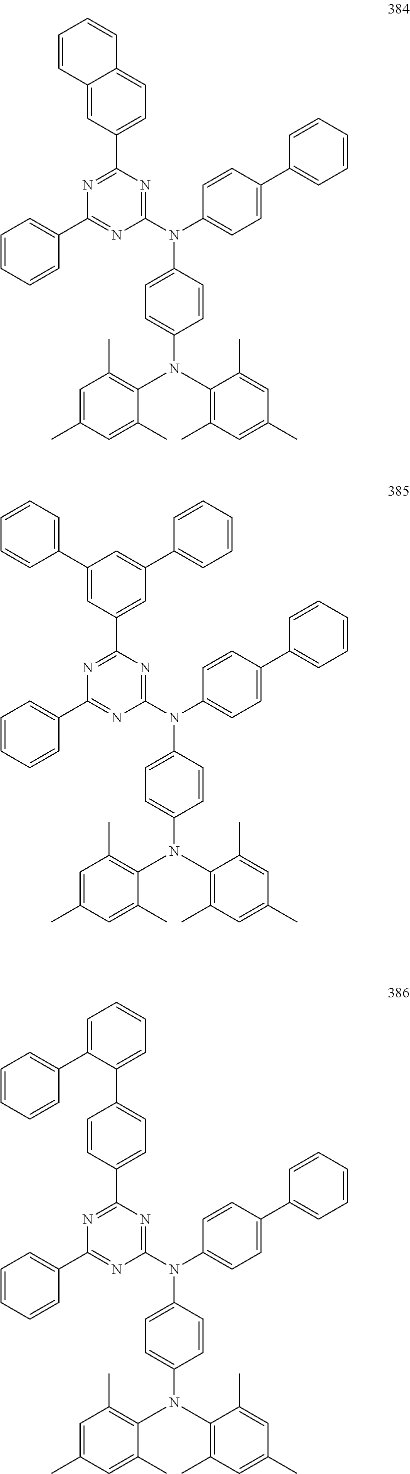 [SMILES: Cc1cc(C)c(N(c2ccc(N(c3ccc(-c4ccccc4)cc3)c3nc(-c4ccccc4)nc(-c4ccc5ccccc5c4)n3)cc2)c2c(C)cc(C)cc2C)c(C)c1 ; Cc1cc(C)c(N(c2ccc(N(c3ccc(-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccc(-c4ccccc4)cc3)c3nc(-c4ccccc4)nc(-c4ccc(-c5ccccc5-c5ccccc5)cc4)n3)cc2)c2c(C)cc(C)cc2C)c(C)c1] 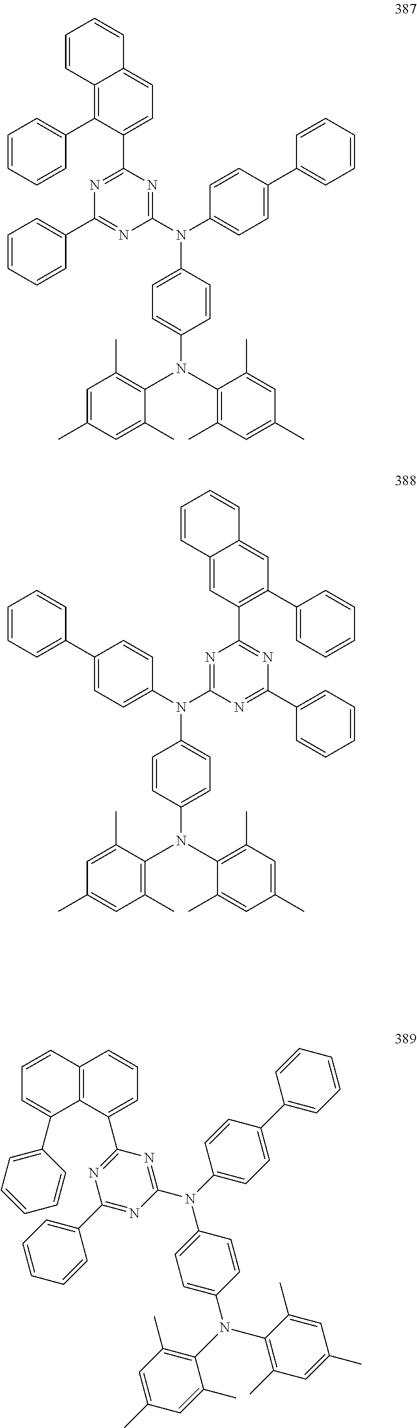 [SMILES: Cc1cc(C)c(N(c2ccc(N(c3ccc(-c4ccccc4)cc3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3ccc(-c4ccccc4)cc3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3ccc(-c4ccccc4)cc3)c3nc(-c4ccccc4)nc(-c4cccc5cccc(-c6ccccc6)c45)n3)cc2)c2c(C)cc(C)cc2C)c(C)c1] 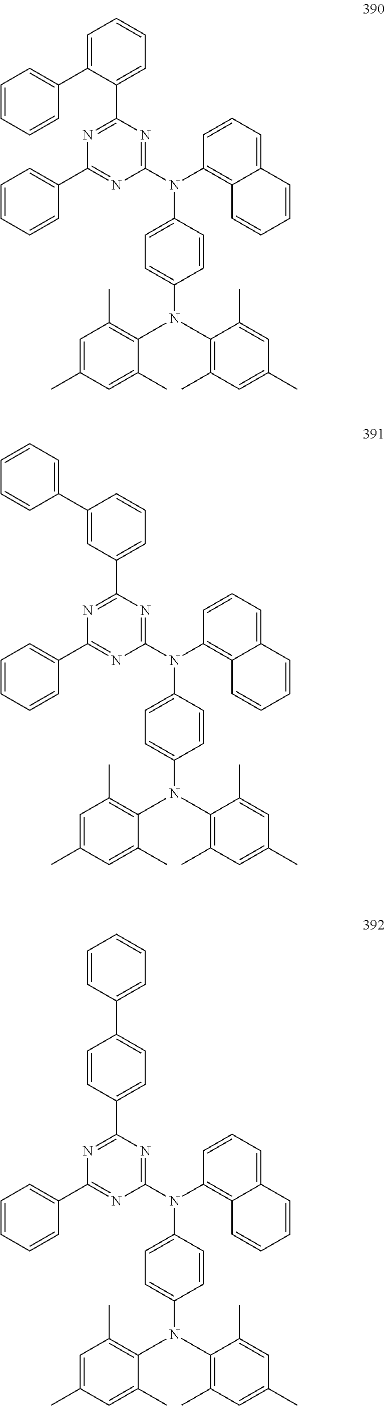 [SMILES: Cc1cc(C)c(N(c2ccc(N(c3nc(-c4ccccc4)nc(-c4ccccc4-c4ccccc4)n3)c3cccc4ccccc34)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3cccc4ccccc34)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3cccc4ccccc34)cc2)c2c(C)cc(C)cc2C)c(C)c1] 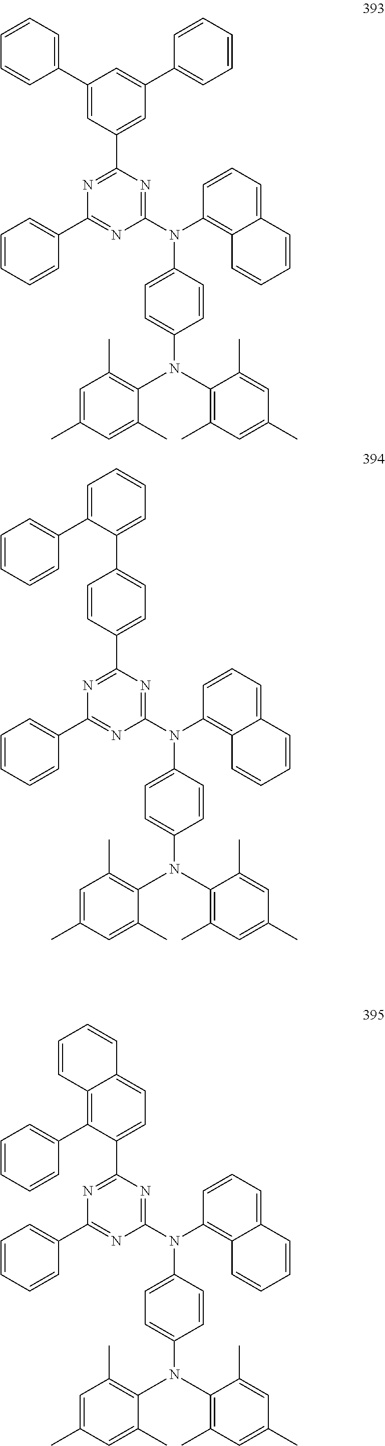 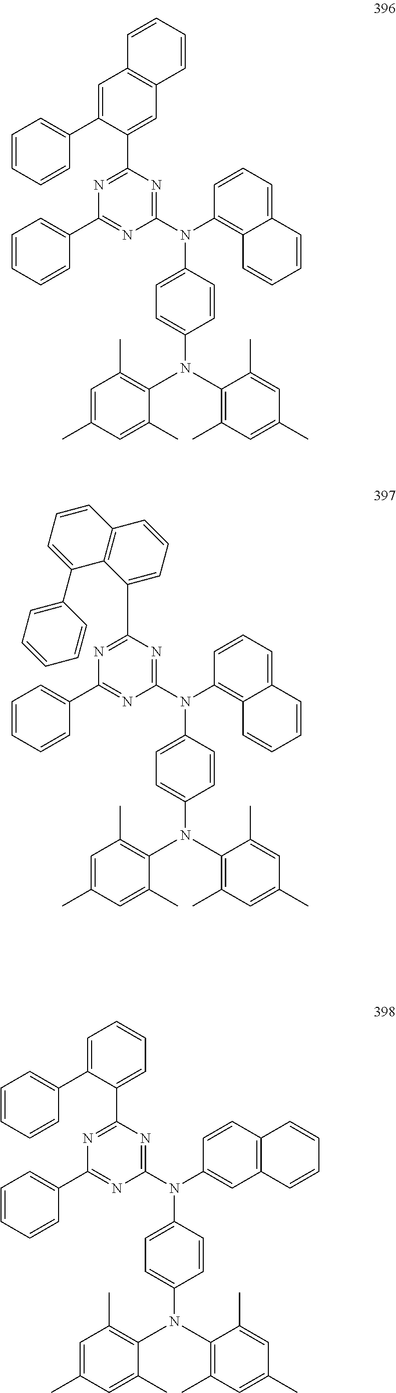 [SMILES: Cc1cc(C)c(N(c2ccc(N(c3nc(-c4ccccc4)nc(-c4cc5ccccc5cc4-c4ccccc4)n3)c3cccc4ccccc34)cc2)c2c(C)cc(C)cc2C)c(C)c1 ; Cc1cc(C)c(N(c2ccc(N(c3nc(-c4ccccc4)nc(-c4cccc5cccc(-c6ccccc6)c45)n3)c3cccc4ccccc34)cc2)c2c(C)cc(C)cc2C)c(C)c1 ; Cc1cc(C)c(N(c2ccc(N(c3ccc4ccccc4c3)c3nc(-c4ccccc4)nc(-c4ccccc4-c4ccccc4)n3)cc2)c2c(C)cc(C)cc2C)c(C)c1] 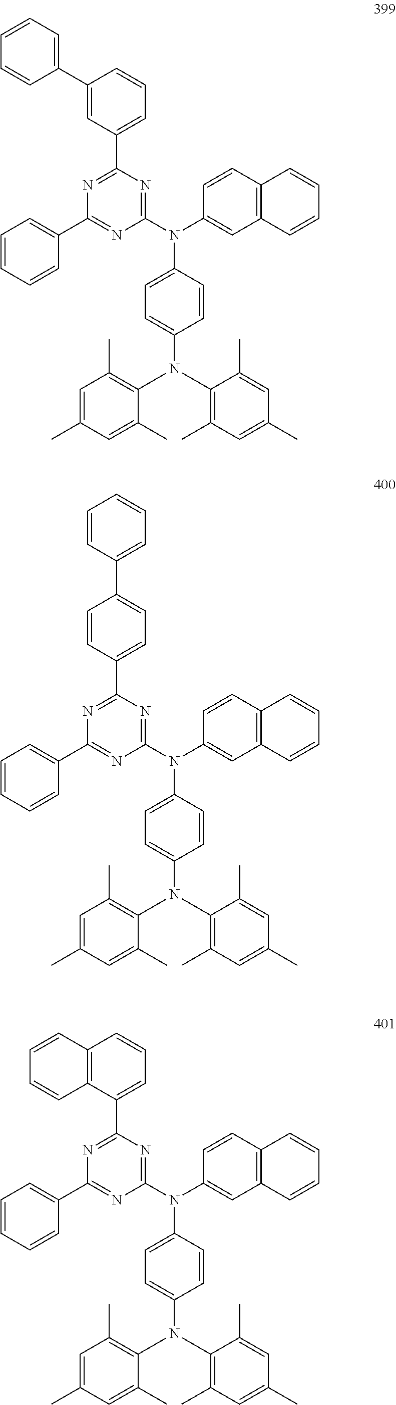 [SMILES: Cc1cc(C)c(N(c2ccc(N(c3ccc4ccccc4c3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3ccc4ccccc4c3)c3nc(-c4ccccc4)nc(-c4ccc(-c5ccccc5)cc4)n3)cc2)c2c(C)cc(C)cc2C)c(C)c1 ; Cc1cc(C)c(N(c2ccc(N(c3ccc4ccccc4c3)c3nc(-c4ccccc4)nc(-c4cccc5ccccc45)n3)cc2)c2c(C)cc(C)cc2C)c(C)c1] 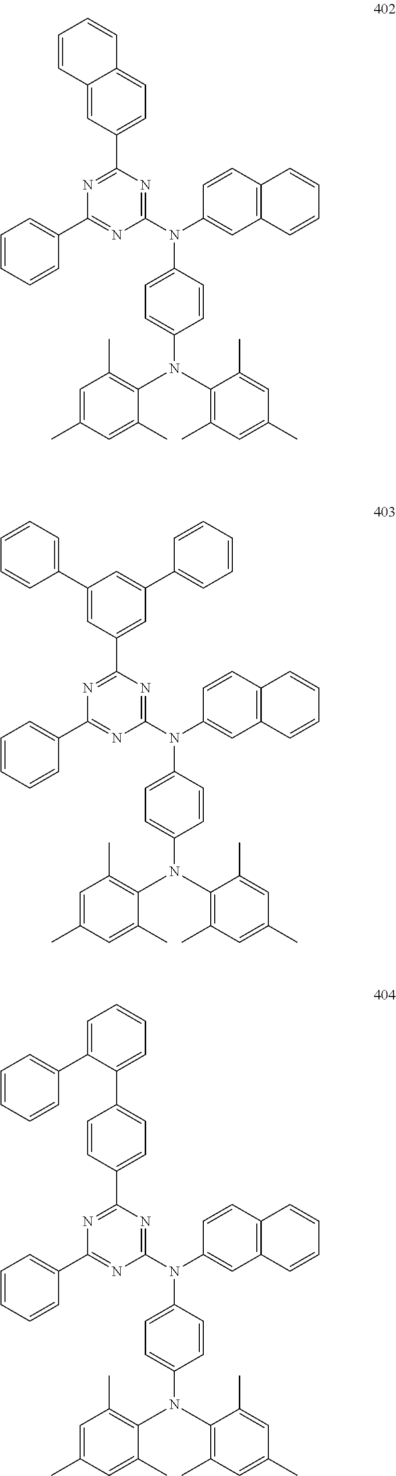 [SMILES: Cc1cc(C)c(N(c2ccc(N(c3ccc4ccccc4c3)c3nc(-c4ccccc4)nc(-c4ccc5ccccc5c4)n3)cc2)c2c(C)cc(C)cc2C)c(C)c1 ; Cc1cc(C)c(N(c2ccc(N(c3ccc4ccccc4c3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccc4ccccc4c3)c3nc(-c4ccccc4)nc(-c4ccc(-c5ccccc5-c5ccccc5)cc4)n3)cc2)c2c(C)cc(C)cc2C)c(C)c1] 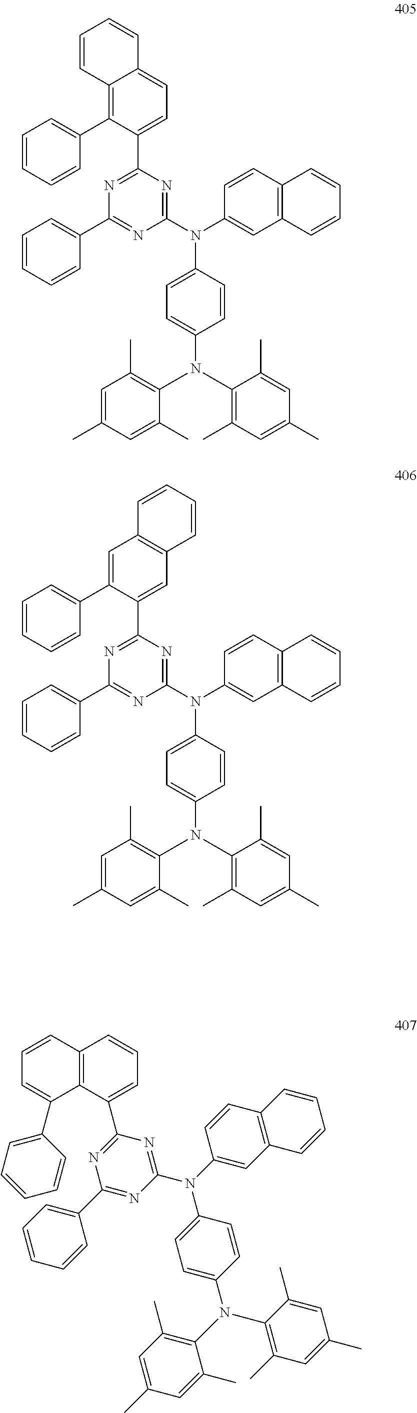 [SMILES: Cc1cc(C)c(N(c2ccc(N(c3ccc4ccccc4c3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3ccc4ccccc4c3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3ccc4ccccc4c3)c3nc(-c4ccccc4)nc(-c4cccc5cccc(-c6ccccc6)c45)n3)cc2)c2c(C)cc(C)cc2C)c(C)c1] 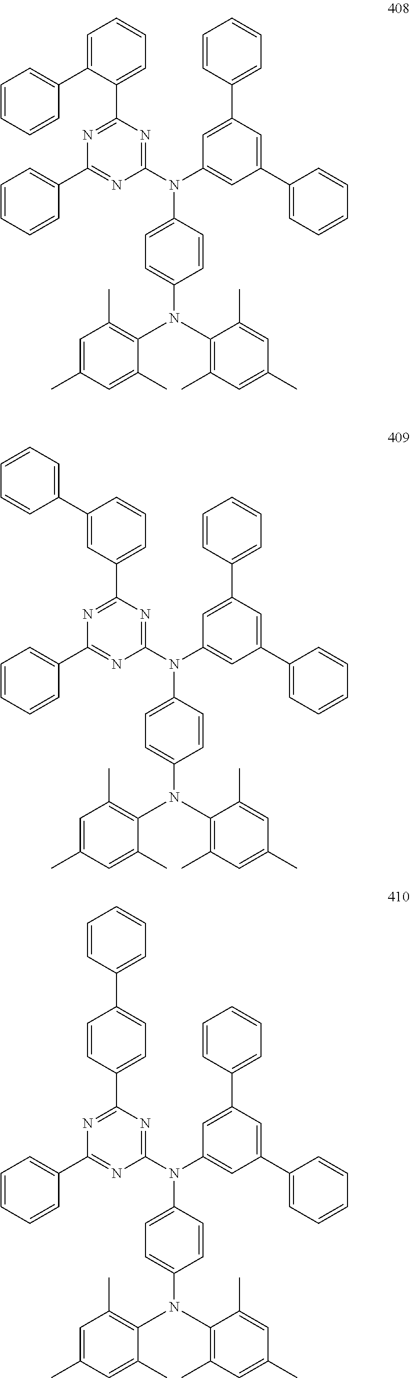 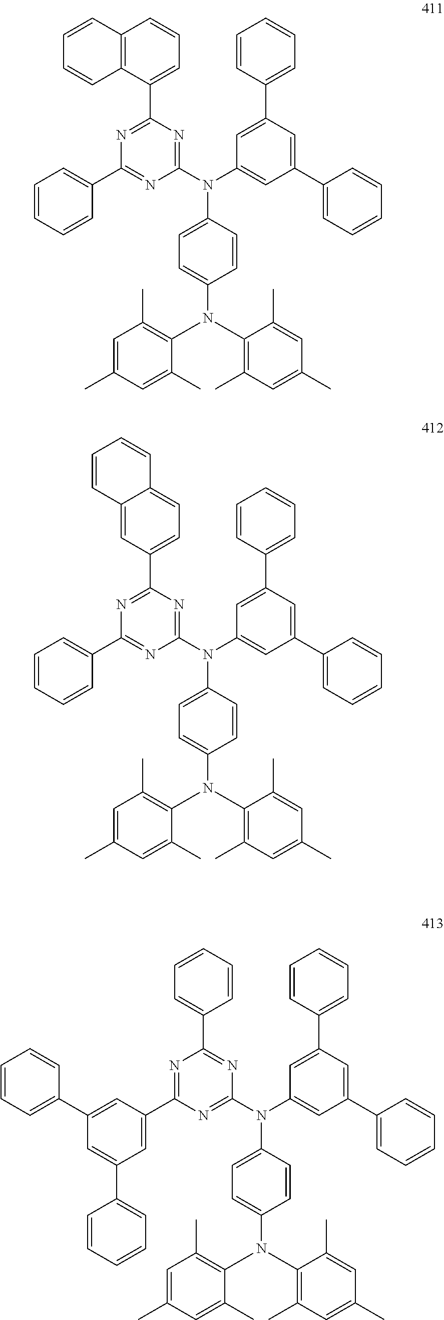 [SMILES: Cc1cc(C)c(N(c2ccc(N(c3cc(-c4ccccc4)cc(-c4ccccc4)c3)c3nc(-c4ccccc4)nc(-c4cccc5ccccc45)n3)cc2)c2c(C)cc(C)cc2C)c(C)c1 ; Cc1cc(C)c(N(c2ccc(N(c3cc(-c4ccccc4)cc(-c4ccccc4)c3)c3nc(-c4ccccc4)nc(-c4ccc5ccccc5c4)n3)cc2)c2c(C)cc(C)cc2C)c(C)c1 ; Cc1cc(C)c(N(c2ccc(N(c3cc(-c4ccccc4)cc(-c4ccccc4)c3)c3nc(-c4ccccc4)nc(-c4cc(-c5ccccc5)cc(-c5ccccc5)c4)n3)cc2)c2c(C)cc(C)cc2C)c(C)c1] 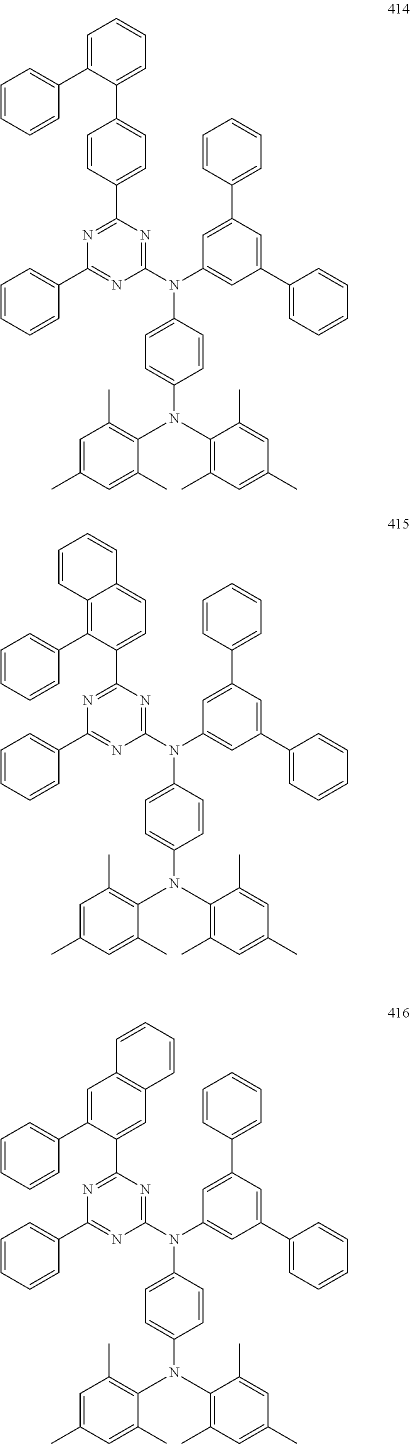 [SMILES: Cc1cc(C)c(N(c2ccc(N(c3cc(-c4ccccc4)cc(-c4ccccc4)c3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(N(c2ccc(N(c3cc(-c4ccccc4)cc(-c4ccccc4)c3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3cc(-c4ccccc4)cc(-c4ccccc4)c3)c3nc(-c4ccccc4)nc(-c4cc5ccccc5cc4-c4ccccc4)n3)cc2)c2c(C)cc(C)cc2C)c(C)c1] 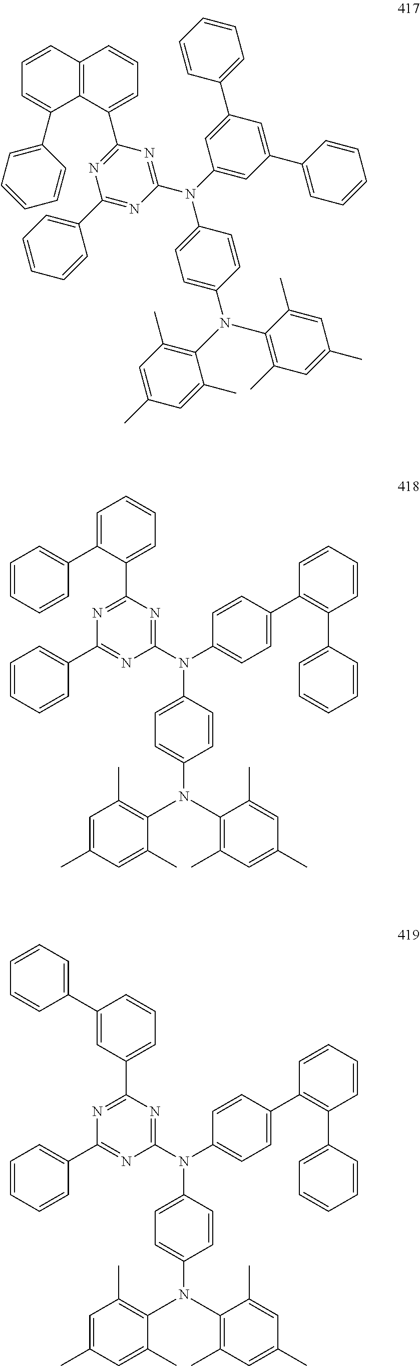 [SMILES: Cc1cc(C)c(N(c2ccc(N(c3cc(-c4ccccc4)cc(-c4ccccc4)c3)c3nc(-c4ccccc4)nc(-c4cccc5cccc(-c6ccccc6)c45)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4cccc(-c5ccccc5)c4)n3)cc2)c2c(C)cc(C)cc2C)c(C)c1] 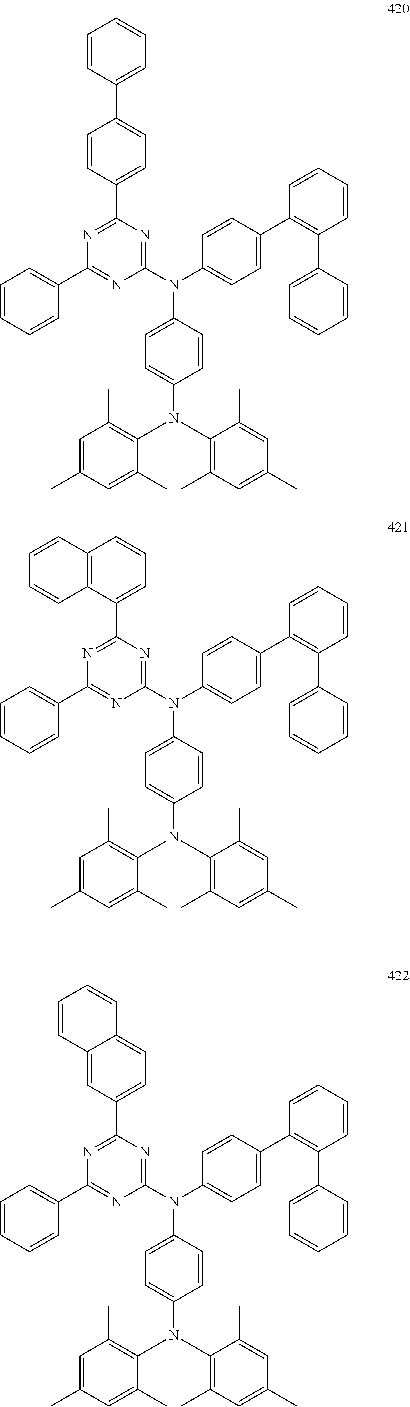 [SMILES: Cc1cc(C)c(N(c2ccc(N(c3ccc(-c4ccccc4-c4ccccc4)cc3)c3nc(-c4ccccc4)nc(-c4ccc(-c5ccccc5)cc4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4cccc5ccccc45)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4ccc5ccccc5c4)n3)cc2)c2c(C)cc(C)cc2C)c(C)c1] 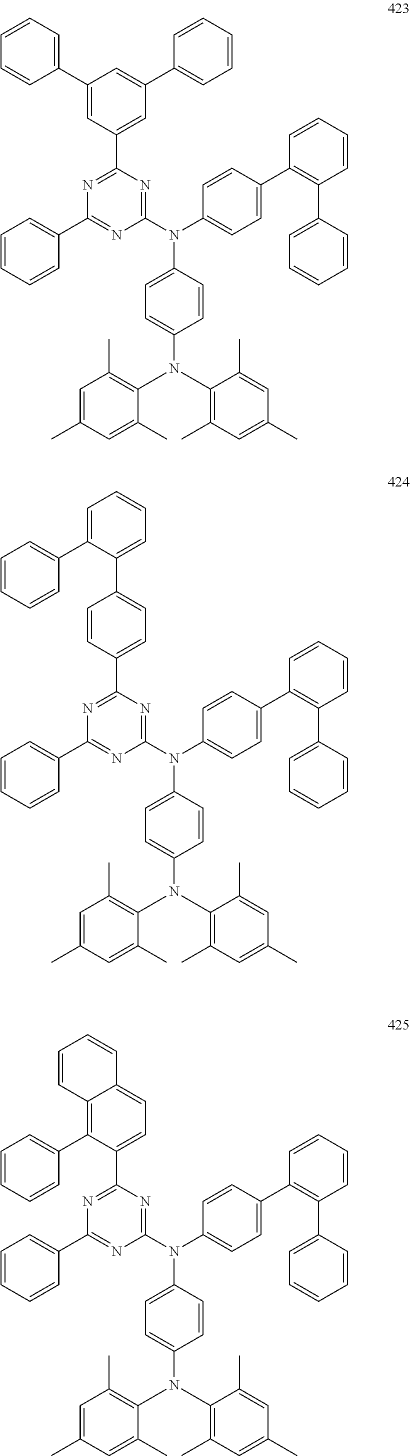 [SMILES: Cc1cc(C)c(N(c2ccc(N(c3ccc(-c4ccccc4-c4ccccc4)cc3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4ccc(-c5ccccc5-c5ccccc5)cc4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4ccc5ccccc5c4-c4ccccc4)n3)cc2)c2c(C)cc(C)cc2C)c(C)c1] 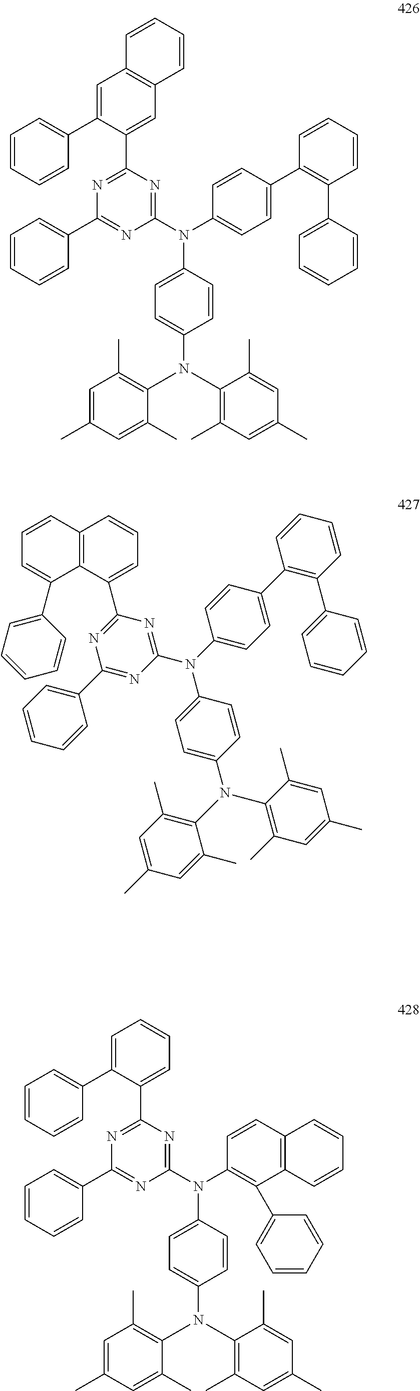 [SMILES: Cc1cc(C)c(N(c2ccc(N(c3ccc(-c4ccccc4-c4ccccc4)cc3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3ccc(-c4ccccc4-c4ccccc4)cc3)c3nc(-c4ccccc4)nc(-c4cccc5cccc(-c6ccccc6)c45)n3)cc2)c2c(C)cc(C)cc2C)c(C)c1 ; Cc1cc(C)c(N(c2ccc(N(c3nc(-c4ccccc4)nc(-c4ccccc4-c4ccccc4)n3)c3ccc4ccccc4c3-c3ccccc3)cc2)c2c(C)cc(C)cc2C)c(C)c1] 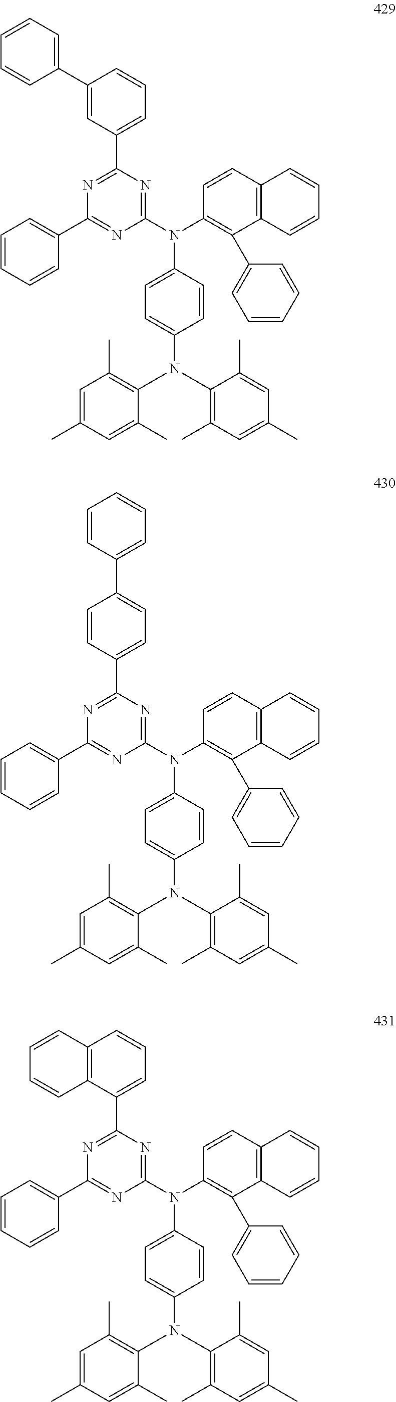 [SMILES: Cc1cc(C)c(N(c2ccc(N(c3nc(-c4ccccc4)nc(-c4cccc(-c5ccccc5)c4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4cccc5ccccc45)n3)c3ccc4ccccc4c3-c3ccccc3)cc2)c2c(C)cc(C)cc2C)c(C)c1] 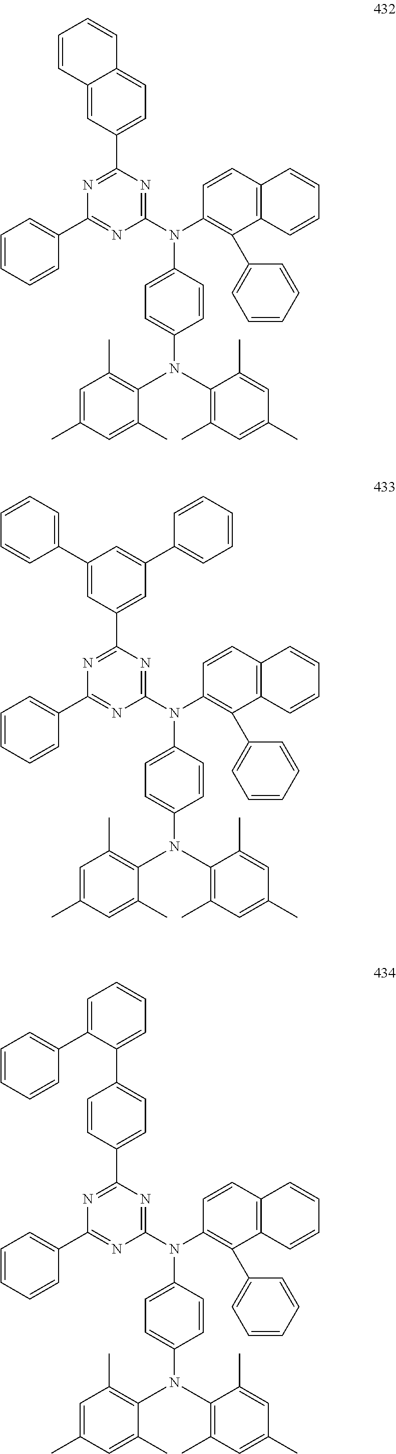 [SMILES: Cc1cc(C)c(N(c2ccc(N(c3nc(-c4ccccc4)nc(-c4ccc5ccccc5c4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4ccc(-c5ccccc5-c5ccccc5)cc4)n3)c3ccc4ccccc4c3-c3ccccc3)cc2)c2c(C)cc(C)cc2C)c(C)c1] 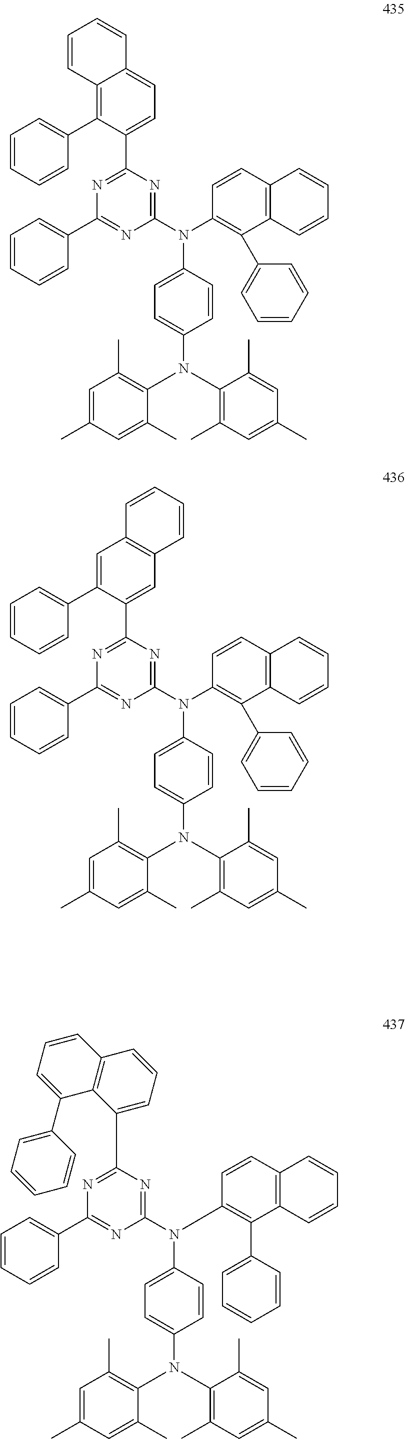 [SMILES: Cc1cc(C)c(N(c2ccc(N(c3nc(-c4ccccc4)nc(-c4ccc5ccccc5c4-c4ccccc4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4cc5ccccc5cc4-c4ccccc4)n3)c3ccc4ccccc4c3-c3ccccc3)cc2)c2c(C)cc(C)cc2C)c(C)c1 ; Cc1cc(C)c(N(c2ccc(N(c3nc(-c4ccccc4)nc(-c4cccc5cccc(-c6ccccc6)c45)n3)c3ccc4ccccc4c3-c3ccccc3)cc2)c2c(C)cc(C)cc2C)c(C)c1] 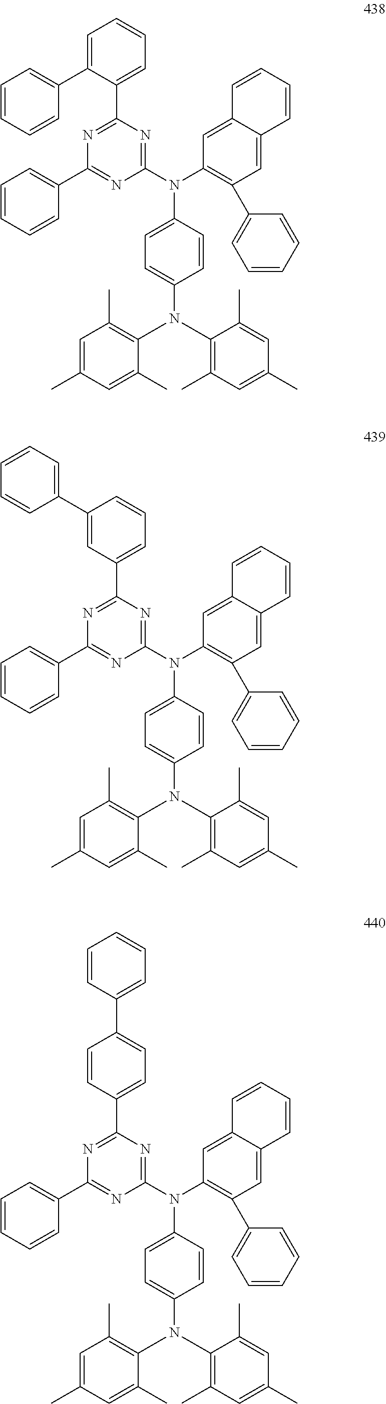 [SMILES: Cc1cc(C)c(N(c2ccc(N(c3nc(-c4ccccc4)nc(-c4ccccc4-c4ccccc4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3cc4ccccc4cc3-c3ccccc3)cc2)c2c(C)cc(C)cc2C)c(C)c1] 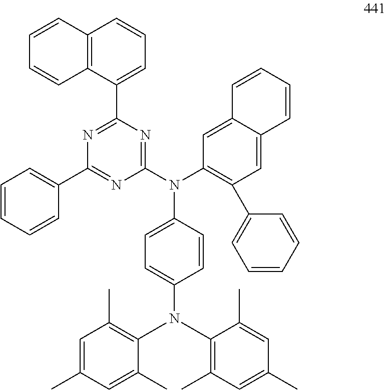 [SMILES: Cc1cc(C)c(N(c2ccc(N(c3nc(-c4ccccc4)nc(-c4cccc5ccccc45)n3)c3cc4ccccc4cc3-c3ccccc3)cc2)c2c(C)cc(C)cc2C)c(C)c1] 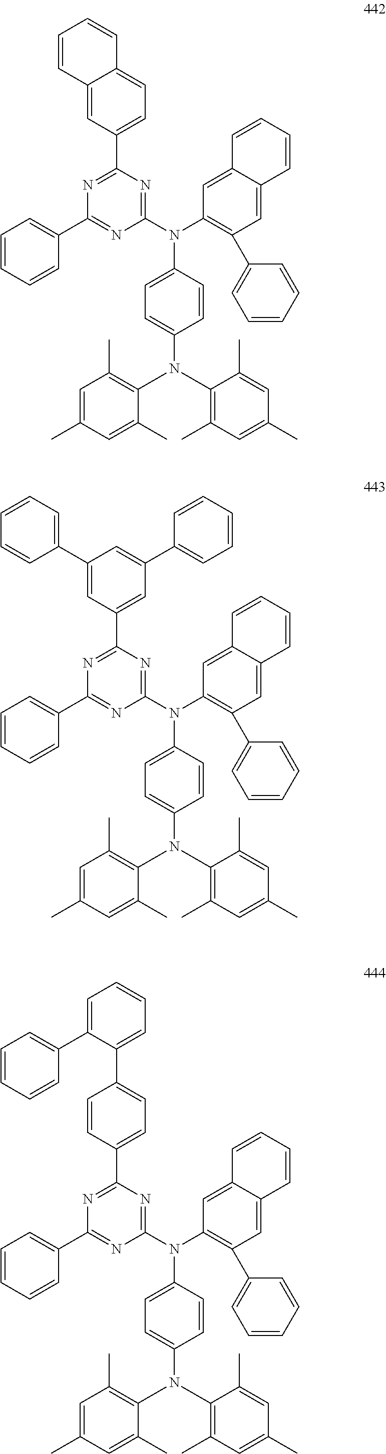 [SMILES: Cc1cc(C)c(N(c2ccc(N(c3nc(-c4ccccc4)nc(-c4ccc5ccccc5c4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4ccc(-c5ccccc5-c5ccccc5)cc4)n3)c3cc4ccccc4cc3-c3ccccc3)cc2)c2c(C)cc(C)cc2C)c(C)c1] 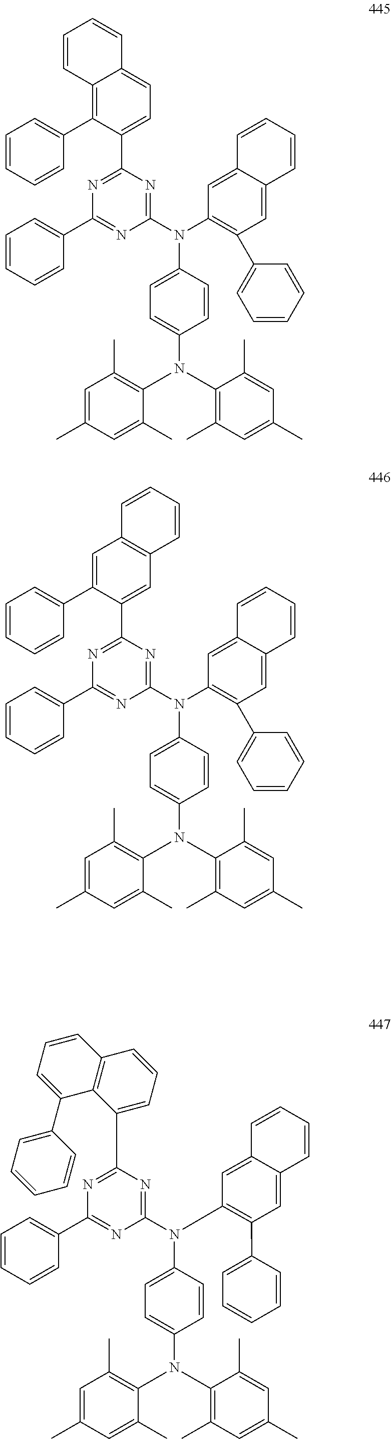 [SMILES: Cc1cc(C)c(N(c2ccc(N(c3nc(-c4ccccc4)nc(-c4ccc5ccccc5c4-c4ccccc4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4cc5ccccc5cc4-c4ccccc4)n3)c3cc4ccccc4cc3-c3ccccc3)cc2)c2c(C)cc(C)cc2C)c(C)c1 ; Cc1cc(C)c(N(c2ccc(N(c3nc(-c4ccccc4)nc(-c4cccc5cccc(-c6ccccc6)c45)n3)c3cc4ccccc4cc3-c3ccccc3)cc2)c2c(C)cc(C)cc2C)c(C)c1] 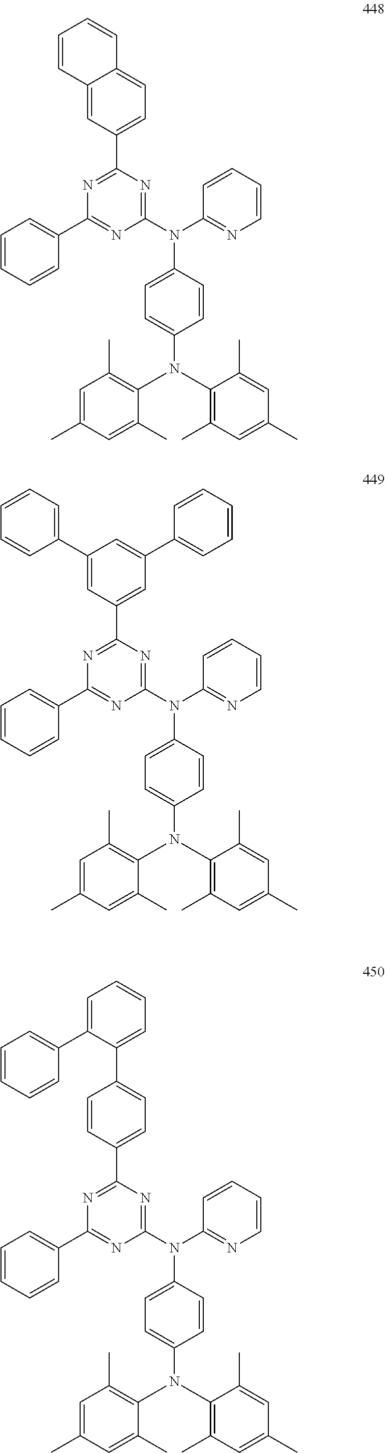 [SMILES: Cc1cc(C)c(N(c2ccc(N(c3ccccn3)c3nc(-c4ccccc4)nc(-c4ccc5ccccc5c4)n3)cc2)c2c(C)cc(C)cc2C)c(C)c1 ; Cc1cc(C)c(N(c2ccc(N(c3ccccn3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccccn3)c3nc(-c4ccccc4)nc(-c4ccc(-c5ccccc5-c5ccccc5)cc4)n3)cc2)c2c(C)cc(C)cc2C)c(C)c1] 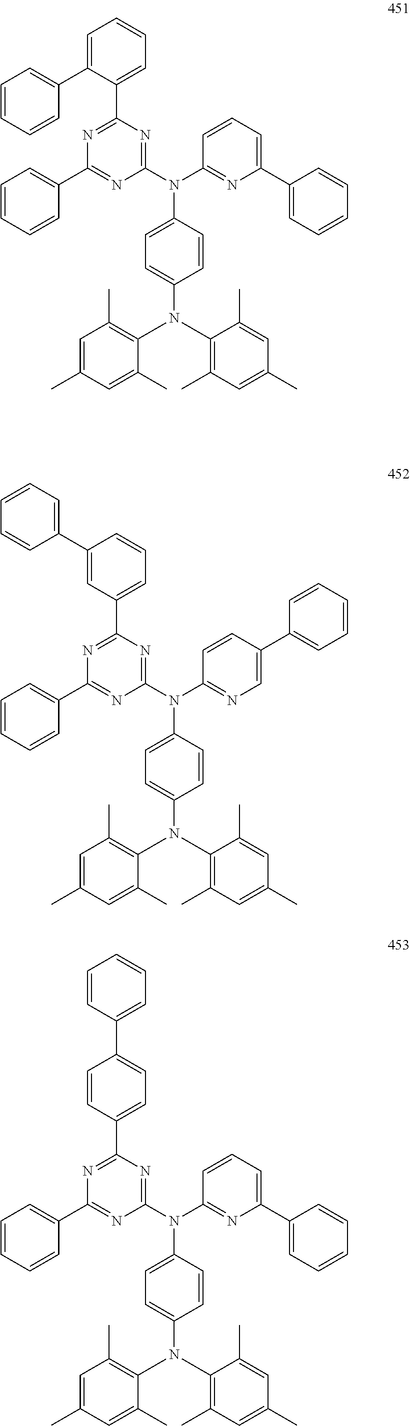 [SMILES: Cc1cc(C)c(N(c2ccc(N(c3cccc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3ccc(-c4ccccc4)cn3)c3nc(-c4ccccc4)nc(-c4cccc(-c5ccccc5)c4)n3)cc2)c2c(C)cc(C)cc2C)c(C)c1 ; Cc1cc(C)c(N(c2ccc(N(c3cccc(-c4ccccc4)n3)c3nc(-c4ccccc4)nc(-c4ccc(-c5ccccc5)cc4)n3)cc2)c2c(C)cc(C)cc2C)c(C)c1] 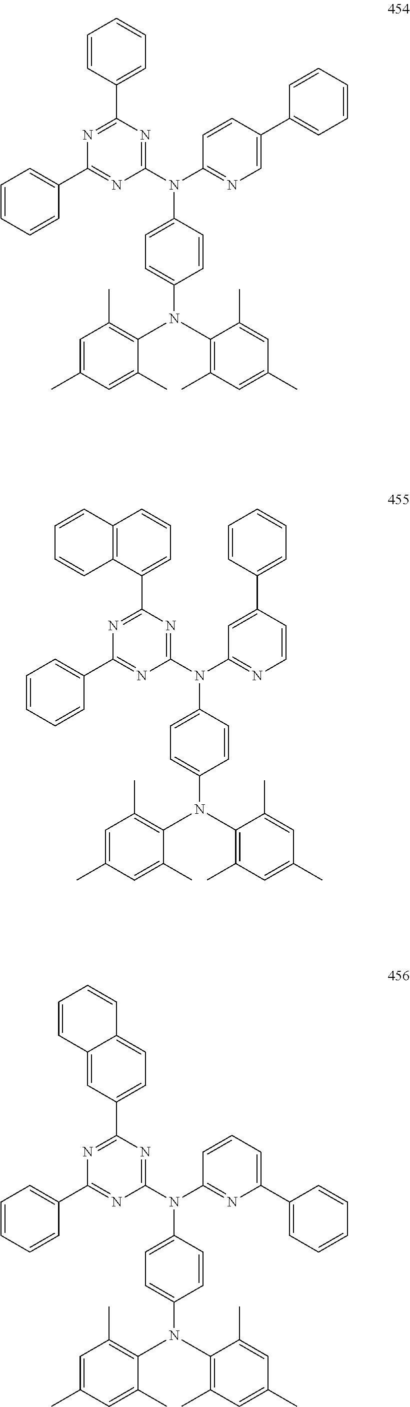 [SMILES: Cc1cc(C)c(N(c2ccc(N(c3ccc(-c4ccccc4)cn3)c3nc(-c4ccccc4)nc(-c4ccccc4)n3)cc2)c2c(C)cc(C)cc2C)c(C)c1 ; Cc1cc(C)c(N(c2ccc(N(c3cc(-c4ccccc4)ccn3)c3nc(-c4ccccc4)nc(-c4cccc5ccccc45)n3)cc2)c2c(C)cc(C)cc2C)c(C)c1 ; Cc1cc(C)c(N(c2ccc(N(c3cccc(-c4ccccc4)n3)c3nc(-c4ccccc4)nc(-c4ccc5ccccc5c4)n3)cc2)c2c(C)cc(C)cc2C)c(C)c1] 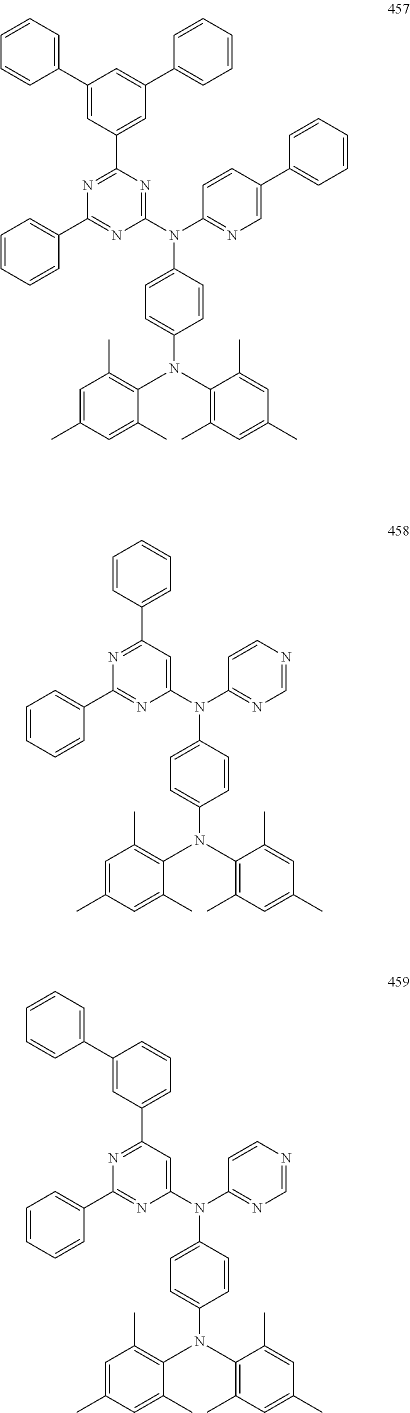 [SMILES: Cc1cc(C)c(N(c2ccc(N(c3ccc(-c4ccccc4)cn3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccncn3)c3cc(-c4ccccc4)nc(-c4ccccc4)n3)cc2)c2c(C)cc(C)cc2C)c(C)c1 ; Cc1cc(C)c(N(c2ccc(N(c3ccncn3)c3cc(-c4cccc(-c5ccccc5)c4)nc(-c4ccccc4)n3)cc2)c2c(C)cc(C)cc2C)c(C)c1] 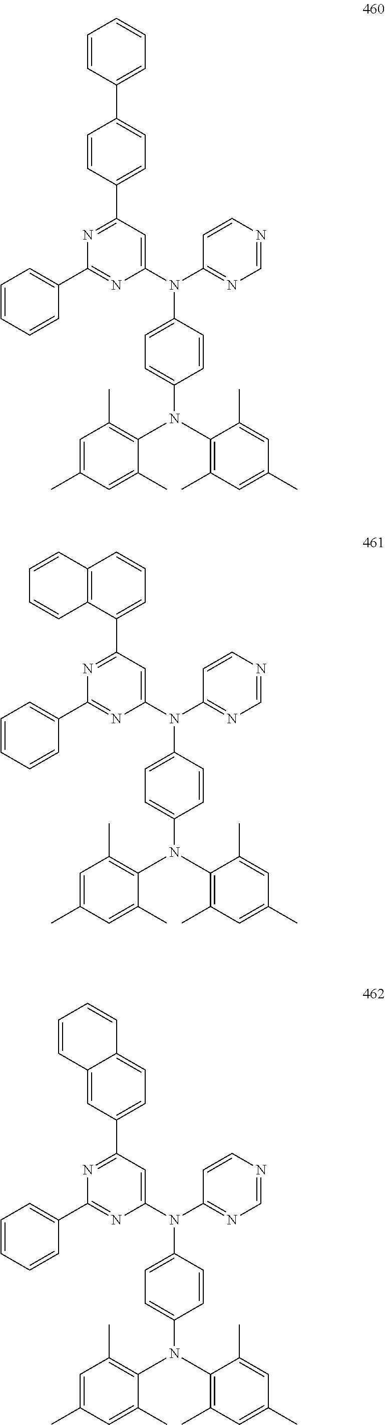 [SMILES: Cc1cc(C)c(N(c2ccc(N(c3ccncn3)c3cc(-c4ccc(-c5ccccc5)cc4)nc(-c4ccccc4)n3)cc2)c2c(C)cc(C)cc2C)c(C)c1 ; Cc1cc(C)c(N(c2ccc(N(c3ccncn3)c3cc(-c4cccc5ccccc45)nc(-c4ccccc4)n3)cc2)c2c(C)cc(C)cc2C)c(C)c1 ; Cc1cc(C)c(N(c2ccc(N(c3ccncn3)c3cc(-c4ccc5ccccc5c4)nc(-c4ccccc4)n3)cc2)c2c(C)cc(C)cc2C)c(C)c1] 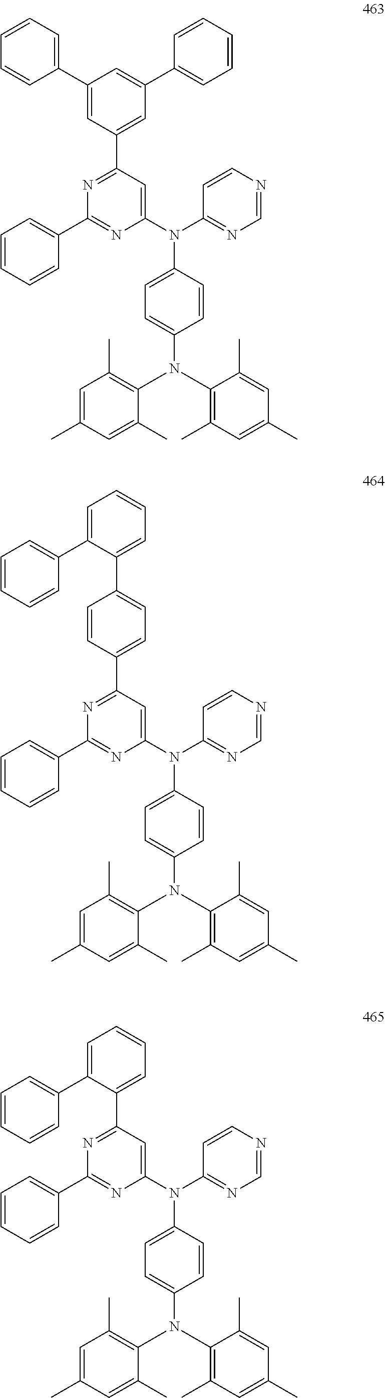 [SMILES: Cc1cc(C)c(N(c2ccc(N(c3ccncn3)c3cc(-c4cc(-c5ccccc5)cc(-c5ccccc5)c4)nc(-c4ccccc4)n3)cc2)c2c(C)cc(C)cc2C)c(C)c1 ; Cc1cc(C)c(N(c2ccc(N(c3ccncn3)c3cc(-c4ccc(-c5ccccc5-c5ccccc5)cc4)nc(-c4ccccc4)n3)cc2)c2c(C)cc(C)cc2C)c(C)c1 ; Cc1cc(C)c(N(c2ccc(N(c3ccncn3)c3cc(-c4ccccc4-c4ccccc4)nc(-c4ccccc4)n3)cc2)c2c(C)cc(C)cc2C)c(C)c1] 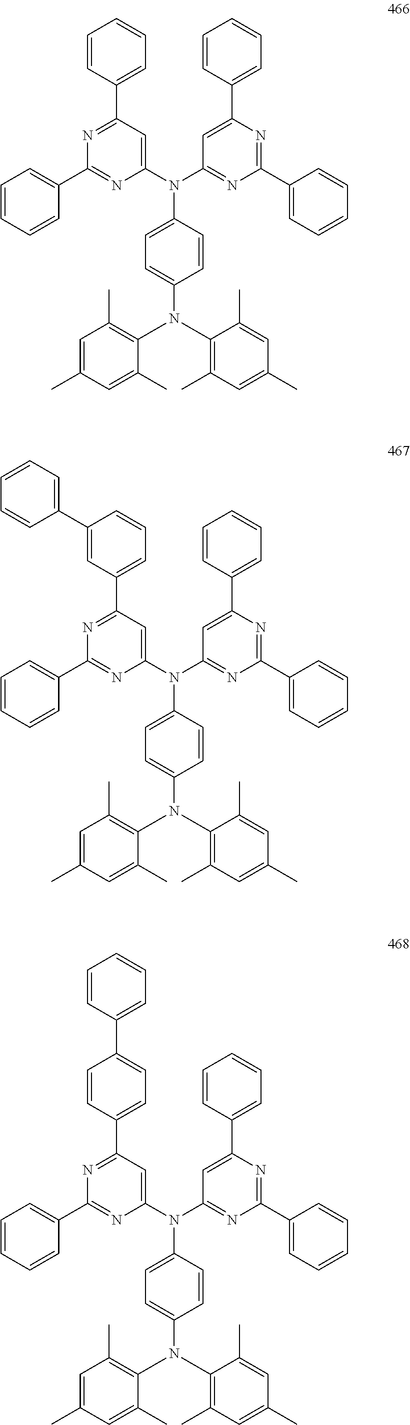 [SMILES: Cc1cc(C)c(N(c2ccc(N(c3cc(-c4ccccc4)nc(-c4ccccc4)n3)c3cc(-c4ccccc4)nc(-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3cc(-c4cccc(-c5ccccc5)c4)nc(-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3cc(-c4ccc(-c5ccccc5)cc4)nc(-c4ccccc4)n3)cc2)c2c(C)cc(C)cc2C)c(C)c1] 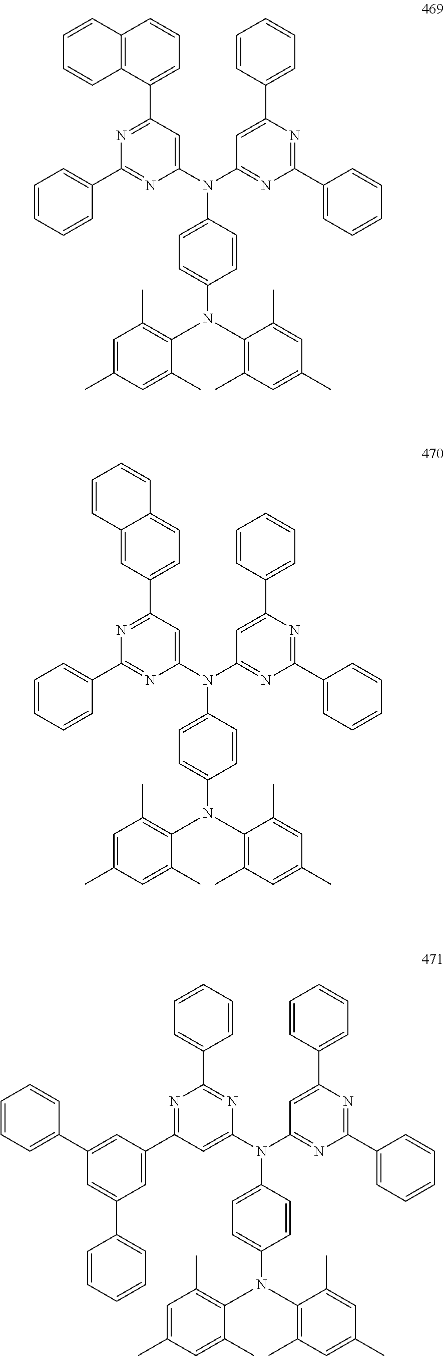 [SMILES: Cc1cc(C)c(N(c2ccc(N(c3cc(-c4ccccc4)nc(-c4ccccc4)n3)c3cc(-c4cccc5ccccc45)nc(-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3cc(-c4ccc5ccccc5c4)nc(-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3cc(-c4cc(-c5ccccc5)cc(-c5ccccc5)c4)nc(-c4ccccc4)n3)cc2)c2c(C)cc(C)cc2C)c(C)c1] 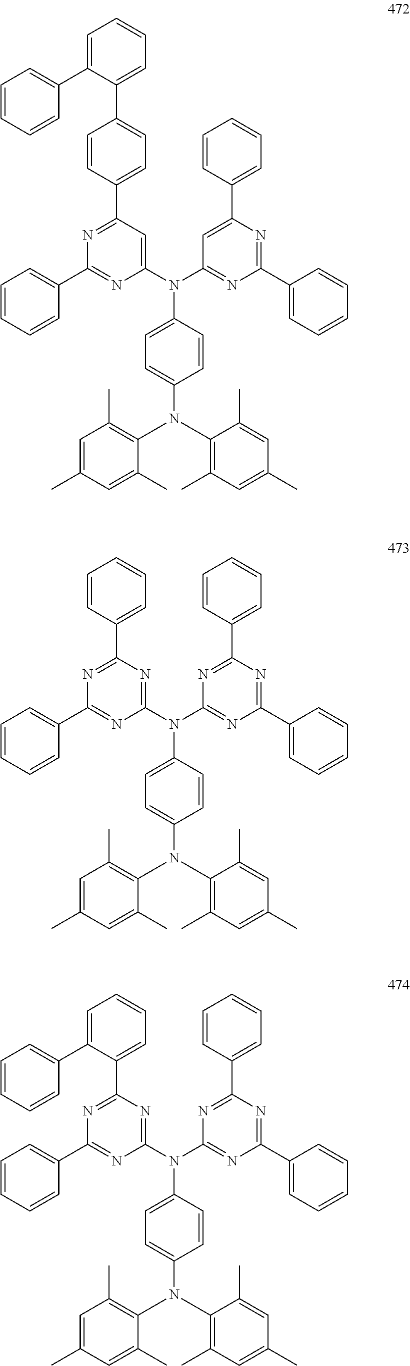 [SMILES: Cc1cc(C)c(N(c2ccc(N(c3cc(-c4ccccc4)nc(-c4ccccc4)n3)c3cc(-c4ccc(-c5ccccc5-c5ccccc5)cc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1] 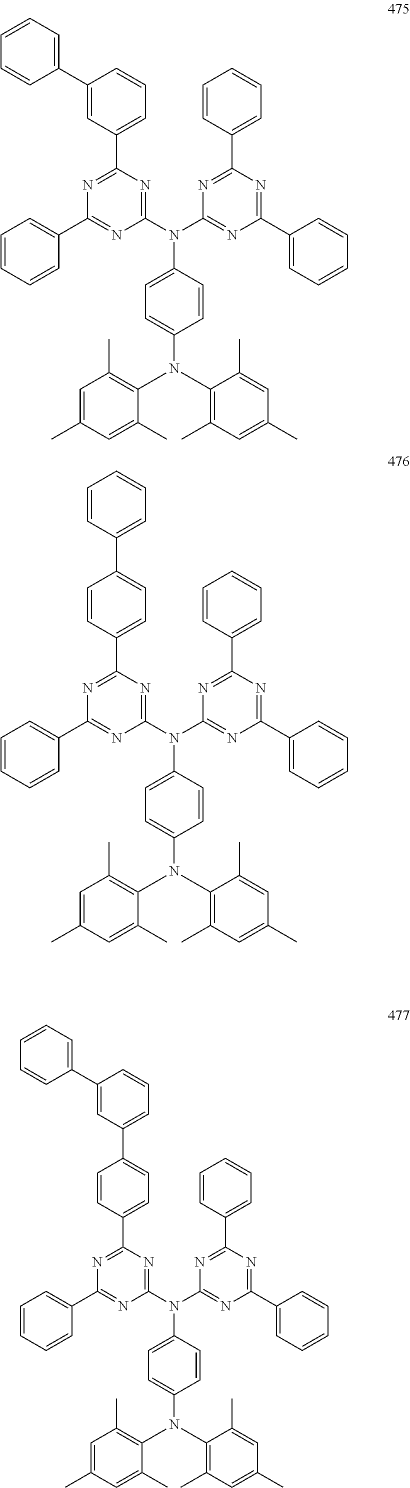 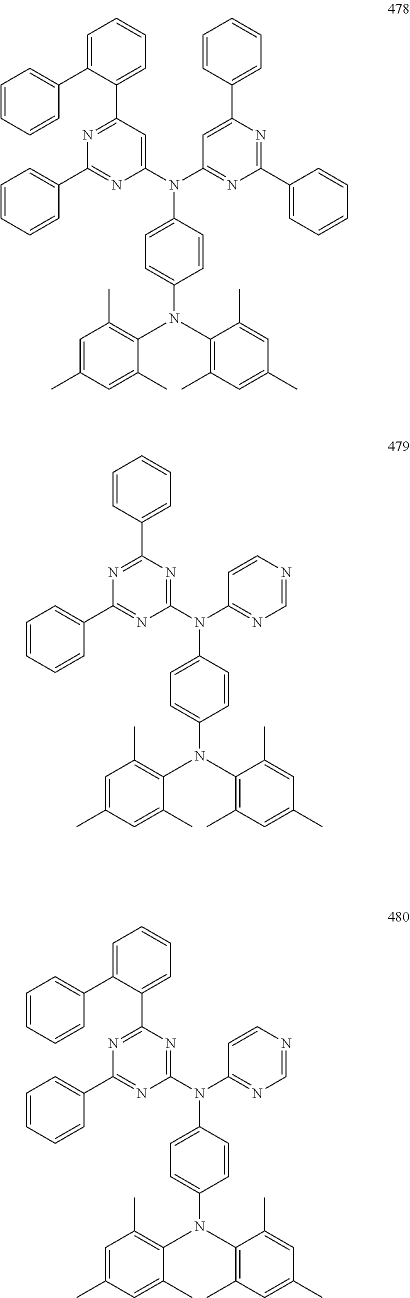 [SMILES: Cc1cc(C)c(N(c2ccc(N(c3cc(-c4ccccc4)nc(-c4ccccc4)n3)c3cc(-c4ccccc4-c4ccccc4)nc(-c4ccccc4)n3)cc2)c2c(C)cc(C)cc2C)c(C)c1 ; Cc1cc(C)c(N(c2ccc(N(c3ccncn3)c3nc(-c4ccccc4)nc(-c4ccccc4)n3)cc2)c2c(C)cc(C)cc2C)c(C)c1 ; Cc1cc(C)c(N(c2ccc(N(c3ccncn3)c3nc(-c4ccccc4)nc(-c4ccccc4-c4ccccc4)n3)cc2)c2c(C)cc(C)cc2C)c(C)c1] 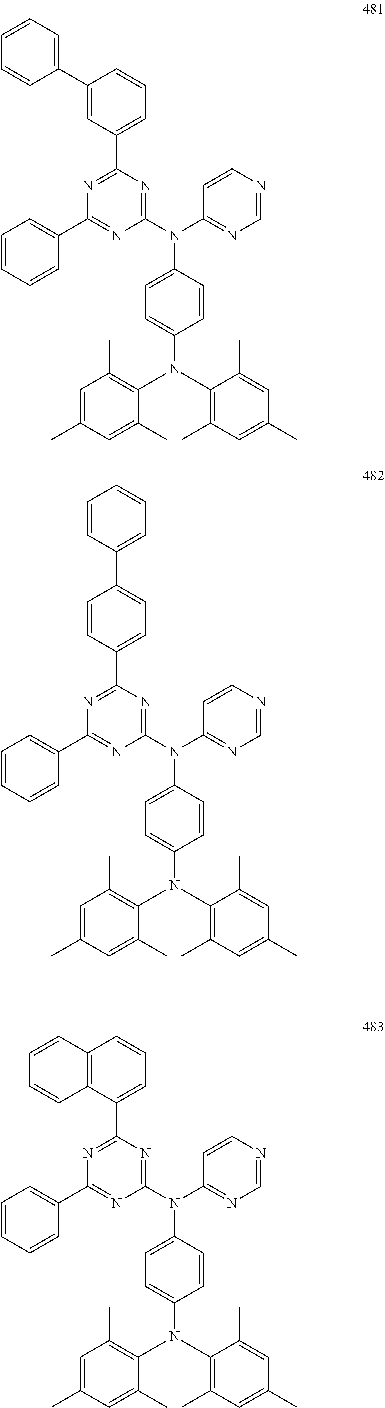 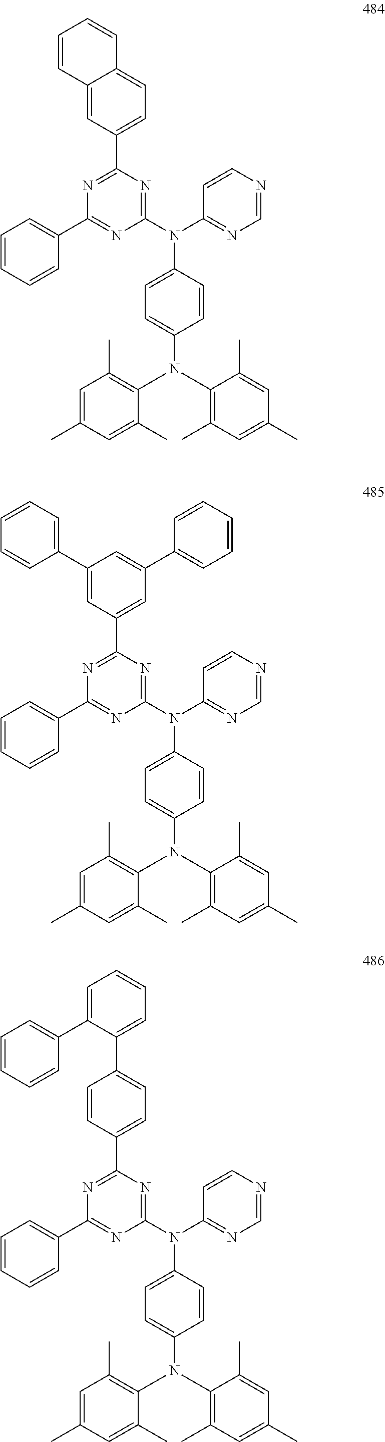 [SMILES: Cc1cc(C)c(N(c2ccc(N(c3ccncn3)c3nc(-c4ccccc4)nc(-c4ccc5ccccc5c4)n3)cc2)c2c(C)cc(C)cc2C)c(C)c1 ; Cc1cc(C)c(N(c2ccc(N(c3ccncn3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3ccncn3)c3nc(-c4ccccc4)nc(-c4ccc(-c5ccccc5-c5ccccc5)cc4)n3)cc2)c2c(C)cc(C)cc2C)c(C)c1] 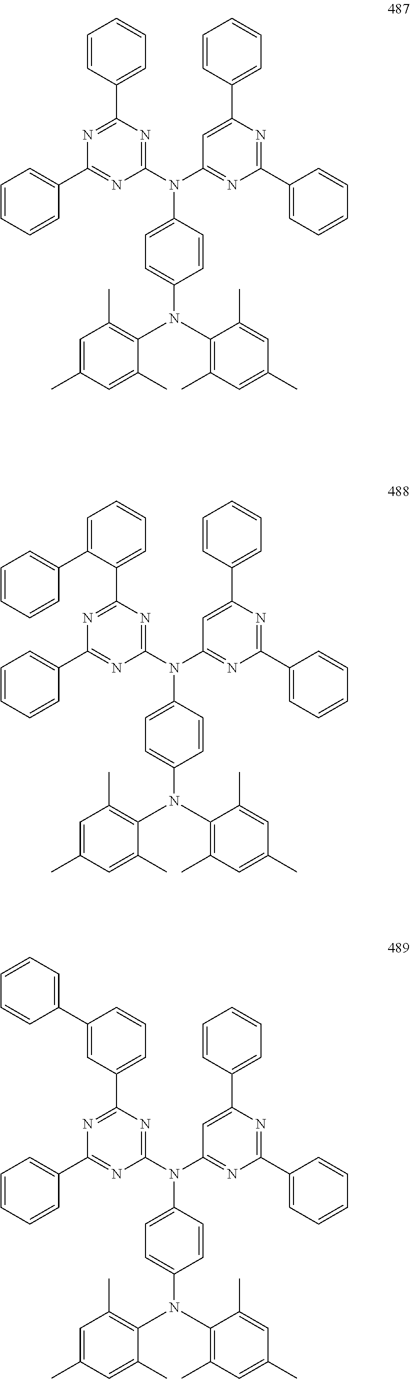 [SMILES: Cc1cc(C)c(N(c2ccc(N(c3cc(-c4ccccc4)nc(-c4ccccc4)n3)c3nc(-c4ccccc4)nc(-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3cc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1] 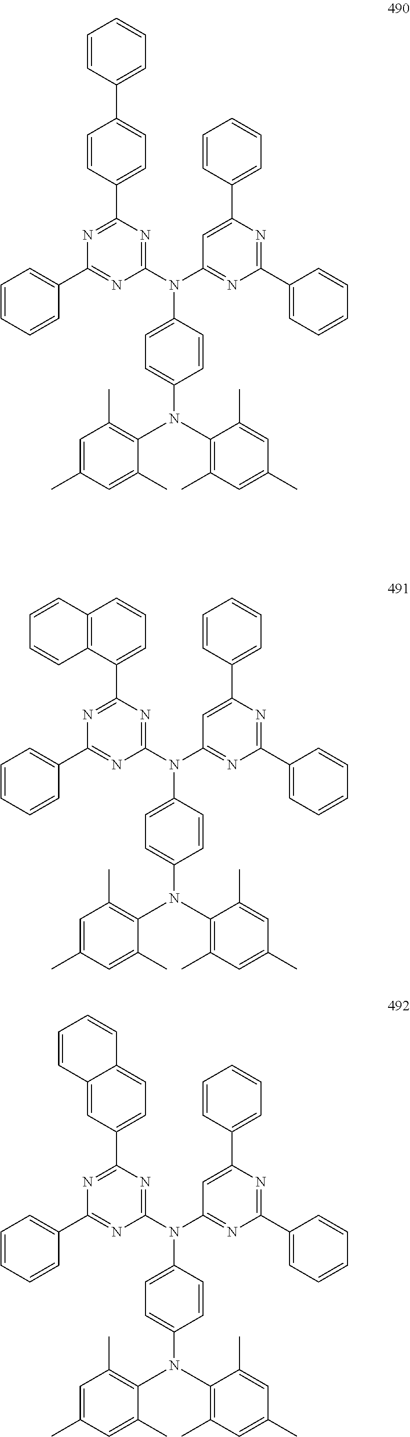 [SMILES: Cc1cc(C)c(N(c2ccc(N(c3cc(-c4ccccc4)nc(-c4ccccc4)n3)c3nc(-c4ccccc4)nc(-c4ccc(-c5ccccc5)cc4)n3)cc2)c2c(C)cc(C)cc2C)c(C)c1 ; Cc1cc(C)c(N(c2ccc(N(c3cc(-c4ccccc4)nc(-c4ccccc4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3cc(-c4ccccc4)nc(-c4ccccc4)n3)c3nc(-c4ccccc4)nc(-c4ccc5ccccc5c4)n3)cc2)c2c(C)cc(C)cc2C)c(C)c1] 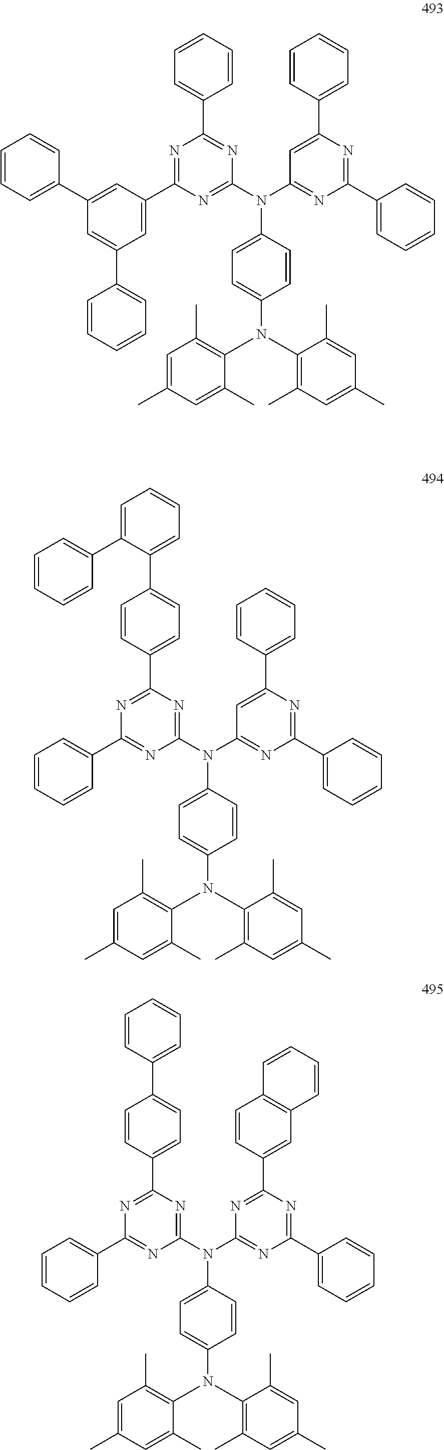 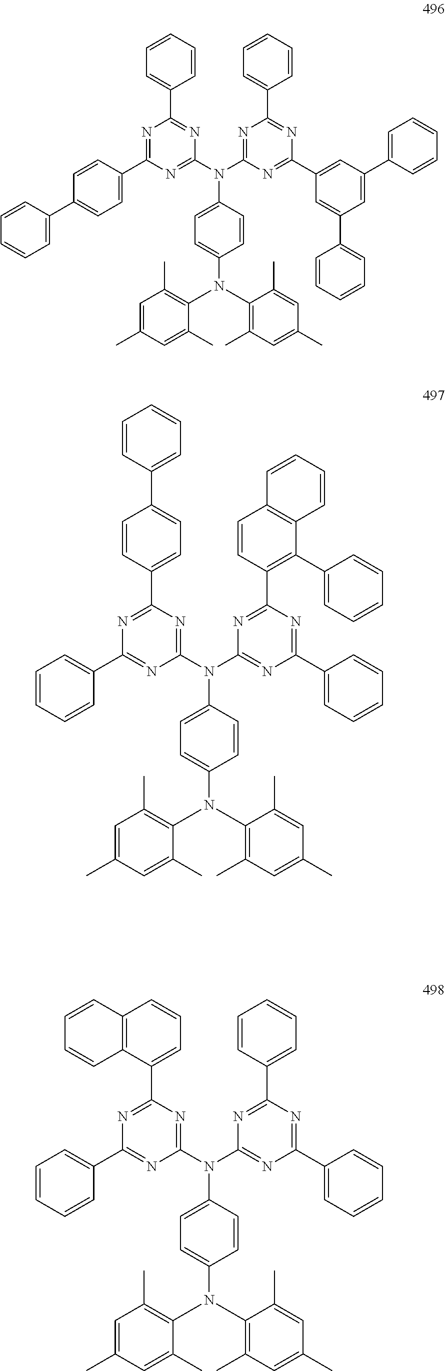 [SMILES: Cc1cc(C)c(N(c2ccc(N(c3nc(-c4ccccc4)nc(-c4ccc(-c5ccccc5)cc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cccc5ccccc45)n3)cc2)c2c(C)cc(C)cc2C)c(C)c1] 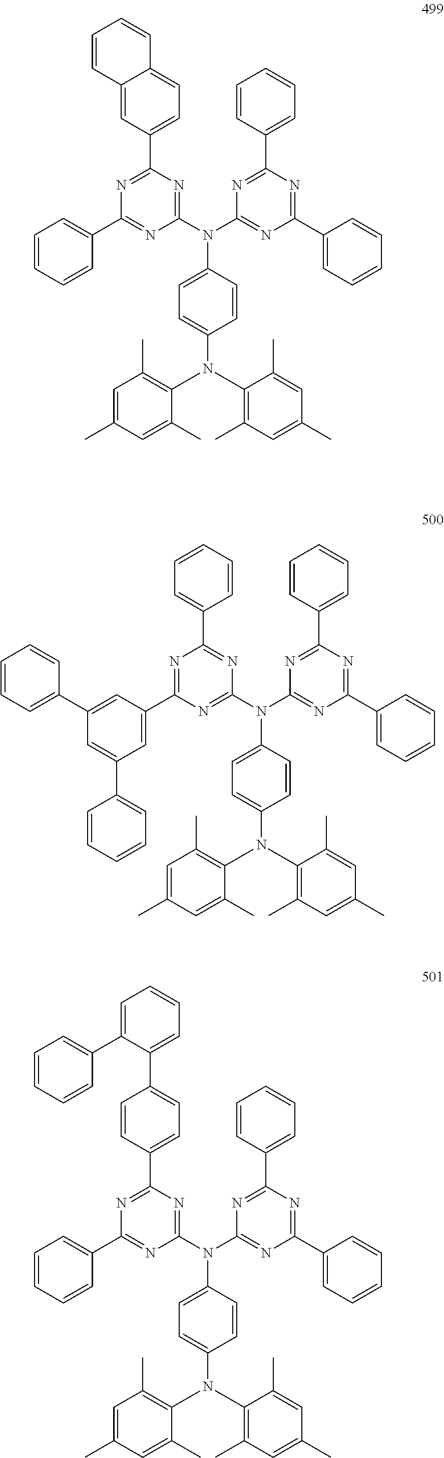 [SMILES: Cc1cc(C)c(N(c2ccc(N(c3nc(-c4ccccc4)nc(-c4ccccc4)n3)c3nc(-c4ccccc4)nc(-c4ccc5ccccc5c4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccc(-c5ccccc5-c5ccccc5)cc4)n3)cc2)c2c(C)cc(C)cc2C)c(C)c1] 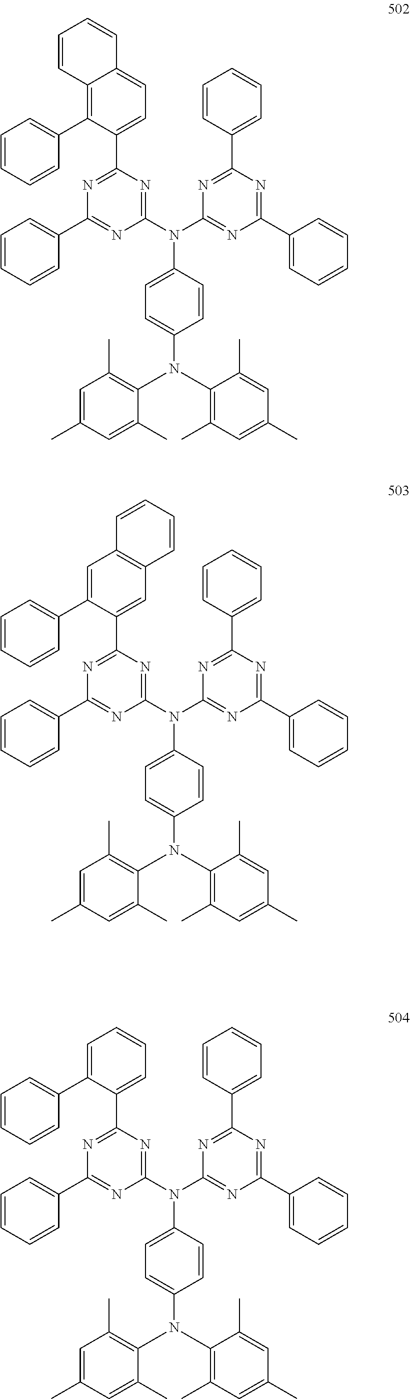 [SMILES: Cc1cc(C)c(N(c2ccc(N(c3nc(-c4ccccc4)nc(-c4ccccc4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)cc(C)cc2C)c(C)c1] 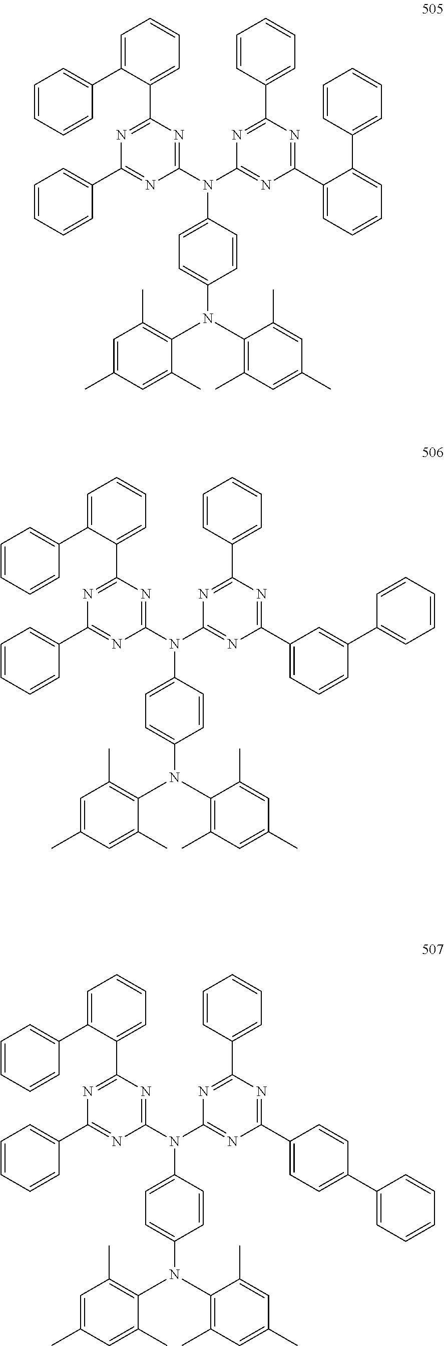 [SMILES: Cc1cc(C)c(N(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)cc(C)cc2C)c(C)c1] 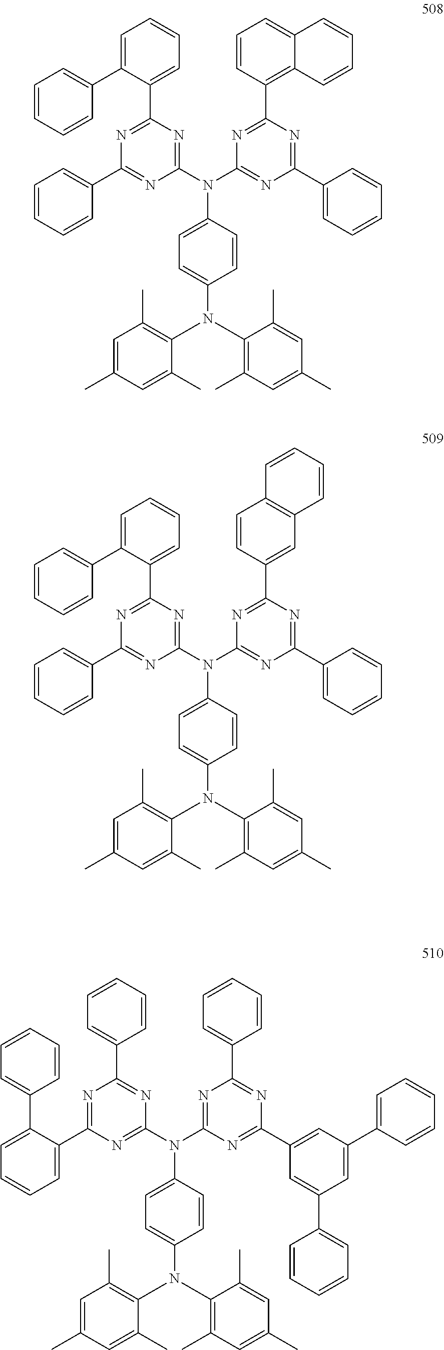 [SMILES: Cc1cc(C)c(N(c2ccc(N(c3nc(-c4ccccc4)nc(-c4ccccc4-c4ccccc4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4ccccc4-c4ccccc4)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccccc4-c4ccccc4)n3)cc2)c2c(C)cc(C)cc2C)c(C)c1] 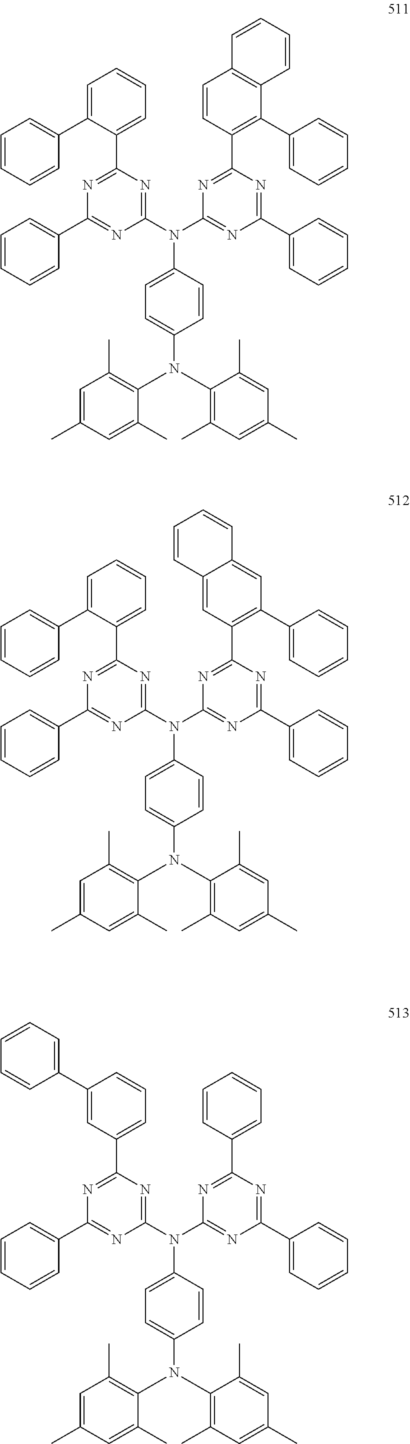 [SMILES: Cc1cc(C)c(N(c2ccc(N(c3nc(-c4ccccc4)nc(-c4ccccc4-c4ccccc4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccccc4-c4ccccc4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cccc(-c5ccccc5)c4)n3)cc2)c2c(C)cc(C)cc2C)c(C)c1] 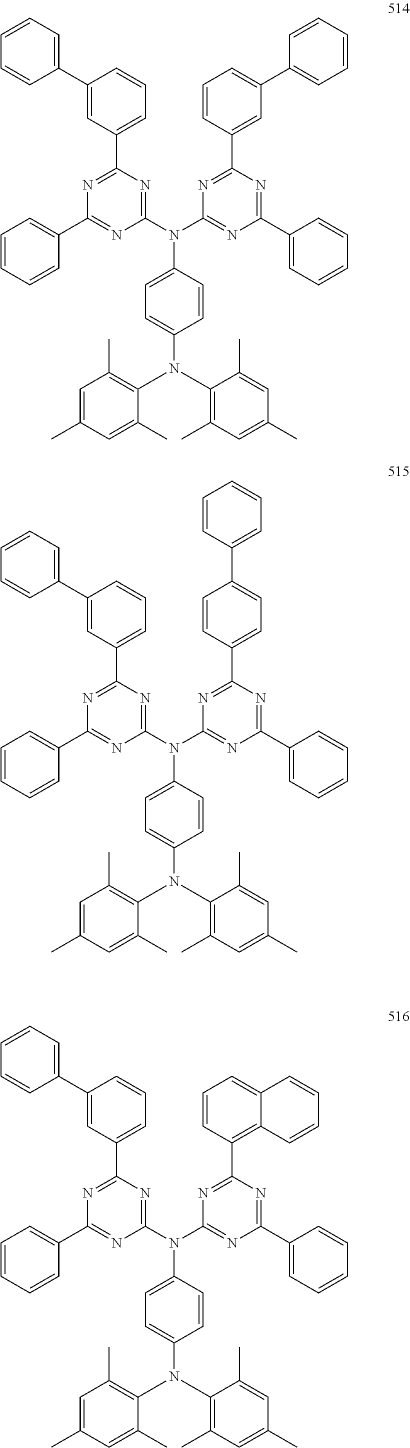 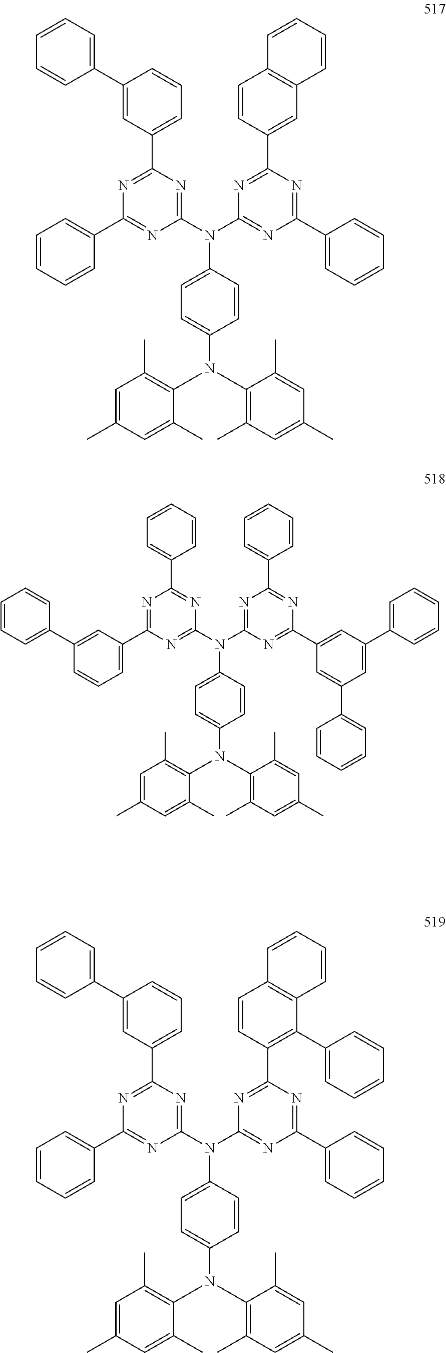 [SMILES: Cc1cc(C)c(N(c2ccc(N(c3nc(-c4ccccc4)nc(-c4cccc(-c5ccccc5)c4)n3)c3nc(-c4ccccc4)nc(-c4ccc5ccccc5c4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4cc(-c5ccccc5)cc(-c5ccccc5)c4)n3)cc2)c2c(C)cc(C)cc2C)c(C)c1 ; Cc1cc(C)c(N(c2ccc(N(c3nc(-c4ccccc4)nc(-c4cccc(-c5ccccc5)c4)n3)c3nc(-c4ccccc4)nc(-c4ccc5ccccc5c4-c4ccccc4)n3)cc2)c2c(C)cc(C)cc2C)c(C)c1] 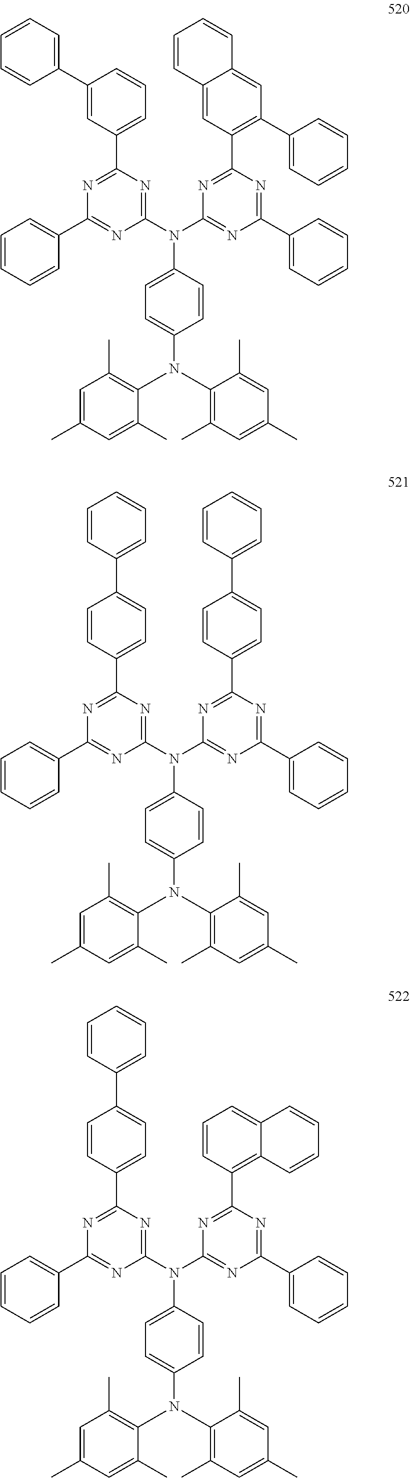 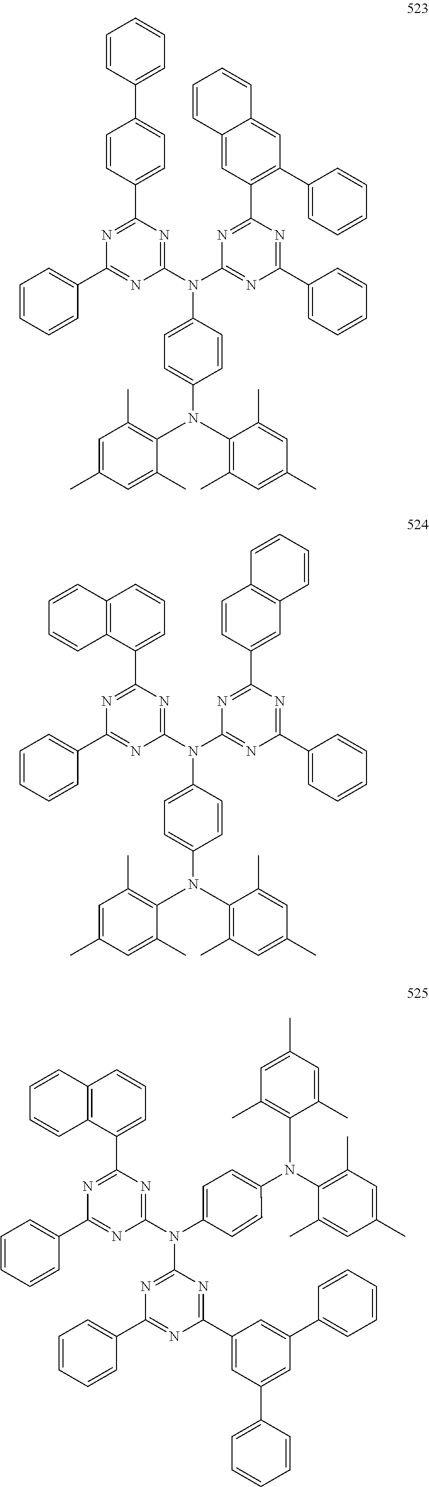 [SMILES: Cc1cc(C)c(N(c2ccc(N(c3nc(-c4ccccc4)nc(-c4ccc(-c5ccccc5)cc4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4cccc5ccccc45)n3)cc2)c2c(C)cc(C)cc2C)c(C)c1] 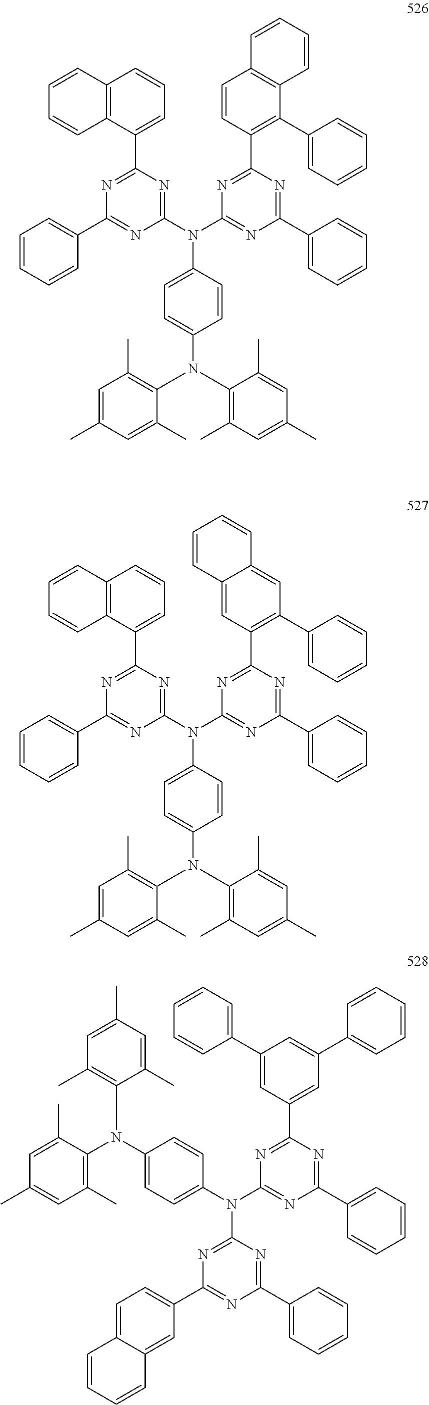 [SMILES: Cc1cc(C)c(N(c2ccc(N(c3nc(-c4ccccc4)nc(-c4ccc5ccccc5c4-c4ccccc4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3nc(-c4ccccc4)nc(-c4cc5ccccc5cc4-c4ccccc4)n3)c3nc(-c4ccccc4)nc(-c4cccc5ccccc45)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4)n3)cc2)c2c(C)cc(C)cc2C)c(C)c1] 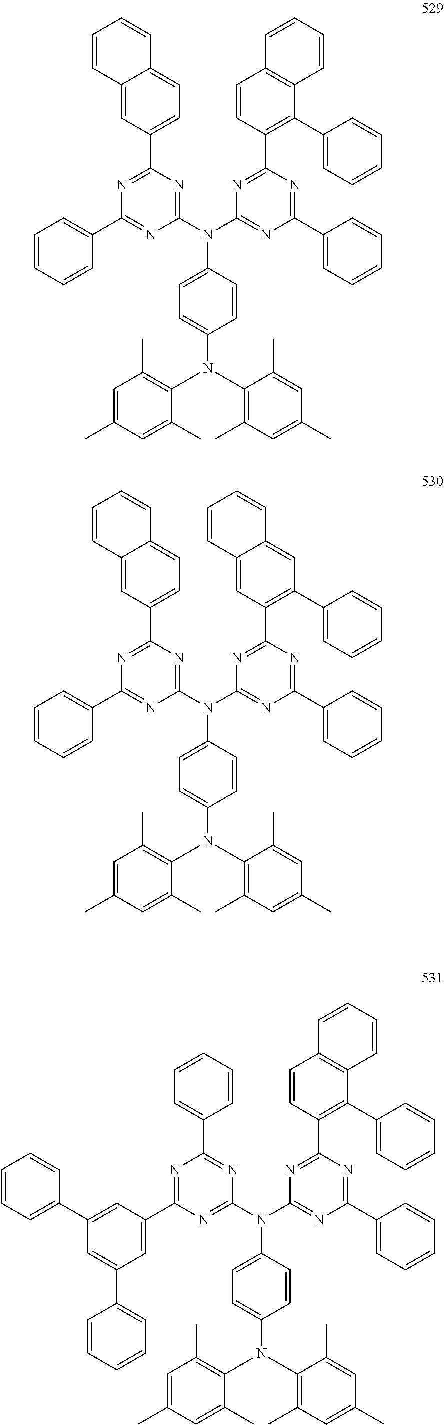 [SMILES: Cc1cc(C)c(N(c2ccc(N(c3nc(-c4ccccc4)nc(-c4ccc5ccccc5c4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2ccc(N(c3nc(-c4ccccc4)nc(-c4ccc5ccccc5c4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4ccc5ccccc5c4-c4ccccc4)n3)cc2)c2c(C)cc(C)cc2C)c(C)c1] 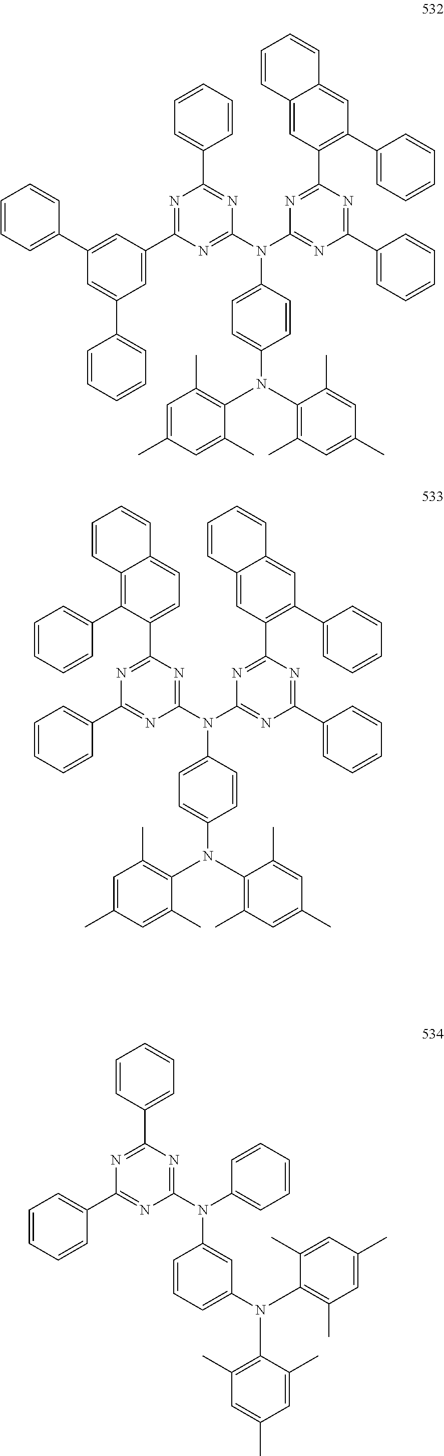 [SMILES: Cc1cc(C)c(N(c2ccc(N(c3nc(-c4ccccc4)nc(-c4cc(-c5ccccc5)cc(-c5ccccc5)c4)n3)c3nc(-c4ccccc4)nc(-c4cc5ccccc5cc4-c4ccccc4)n3)cc2)c2c(C)cc(C)cc2C)c(C)c1 ; Cc1cc(C)c(N(c2ccc(N(c3nc(-c4ccccc4)nc(-c4cc5ccccc5cc4-c4ccccc4)n3)c3nc(-c4ccccc4)nc(-c4ccc5ccccc5c4-c4ccccc4)n3)cc2)c2c(C)cc(C)cc2C)c(C)c1 ; Cc1cc(C)c(N(c2cccc(N(c3ccccc3)c3nc(-c4ccccc4)nc(-c4ccccc4)n3)c2)c2c(C)cc(C)cc2C)c(C)c1] 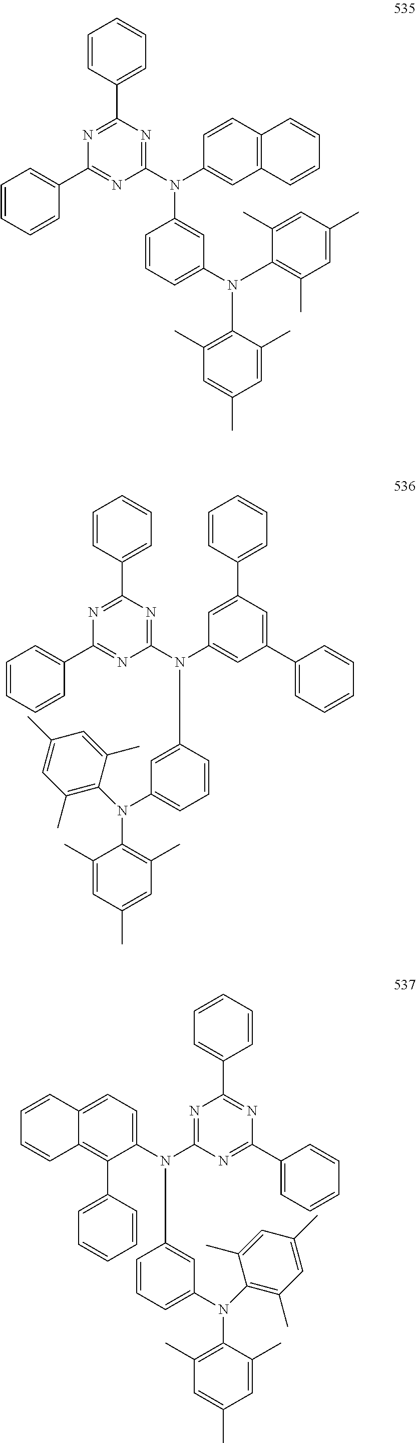 [SMILES: Cc1cc(C)c(N(c2cccc(N(c3ccc4ccccc4c3)c3nc(-c4ccccc4)nc(-c4ccccc4)n3)c2)c2c(C)cc(C)cc2C)c(C)c1 ; Cc1cc(C)c(N(c2cccc(N(c3cc(-c4ccccc4)cc(-c4ccccc4)c3)c3nc(-c4ccccc4)nc(-c4ccccc4)n3)c2)c2c(C)cc(C)cc2C)c(C)c1 ; Cc1cc(C)c(N(c2cccc(N(c3nc(-c4ccccc4)nc(-c4ccccc4)n3)c3ccc4ccccc4c3-c3ccccc3)c2)c2c(C)cc(C)cc2C)c(C)c1] 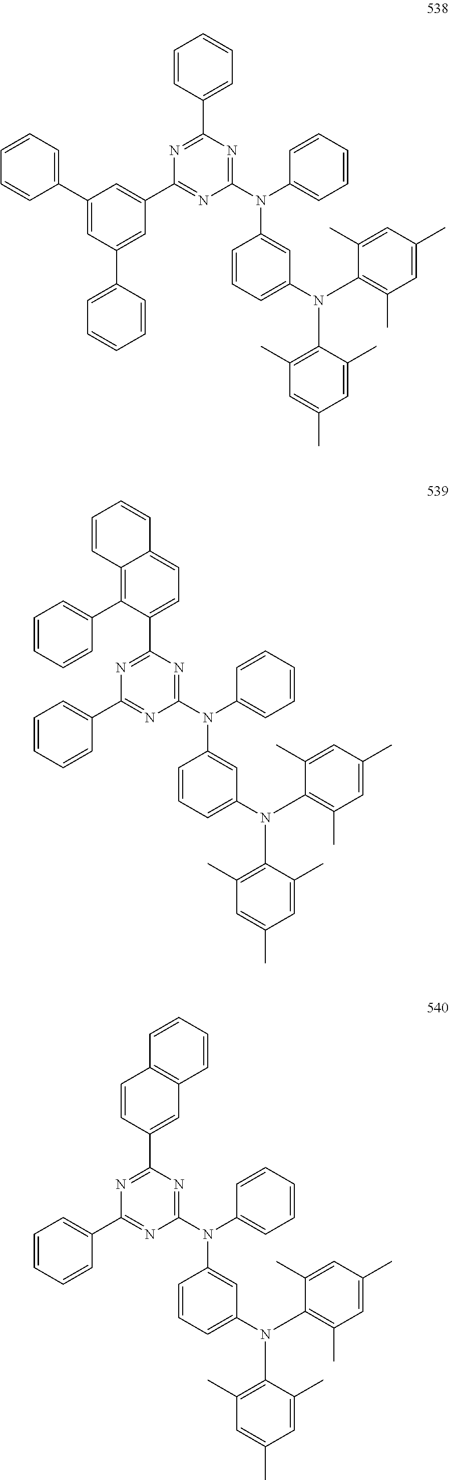 [SMILES: Cc1cc(C)c(N(c2cccc(N(c3ccccc3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4-c4ccccc4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4)n3)c2)c2c(C)cc(C)cc2C)c(C)c1] 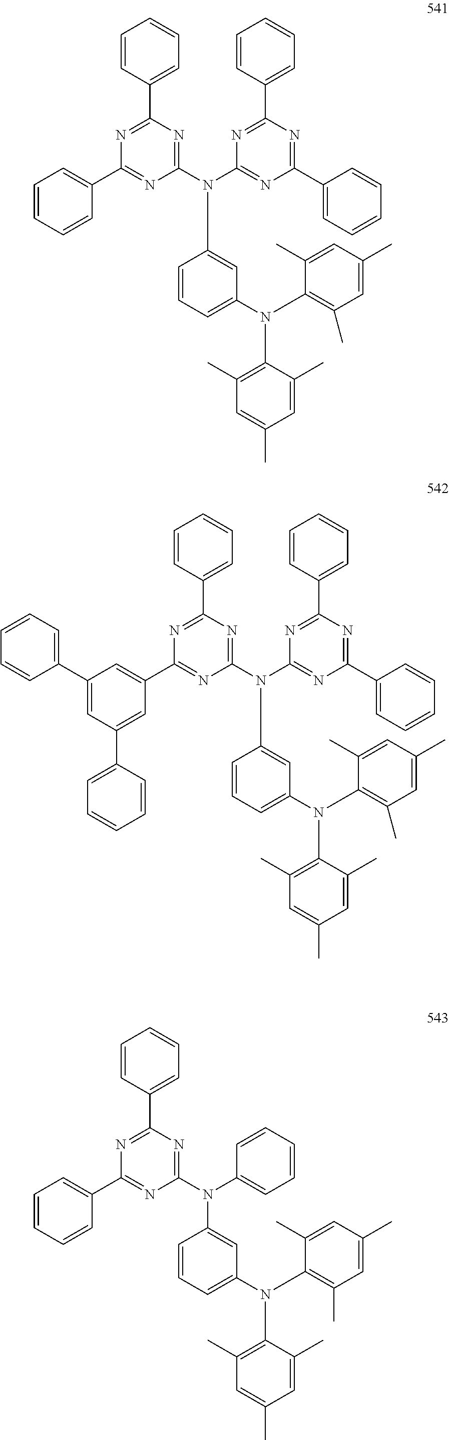 [SMILES: Cc1cc(C)c(N(c2cccc(N(c3nc(-c4ccccc4)nc(-c4ccccc4)n3)c3nc(-c4ccccc4)nc(-c4ccccc4)n3)c2)c2c(C)cc(C)cc2C)c(C)c1 ; Cc1cc(C)c(N(c2cccc(N(c3nc(-c4ccccc4)nc(-c4ccccc4)n3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2cccc(N(c3ccccc3)c3nc(-c4ccccc4)nc(-c4ccccc4)n3)c2)c2c(C)cc(C)cc2C)c(C)c1] 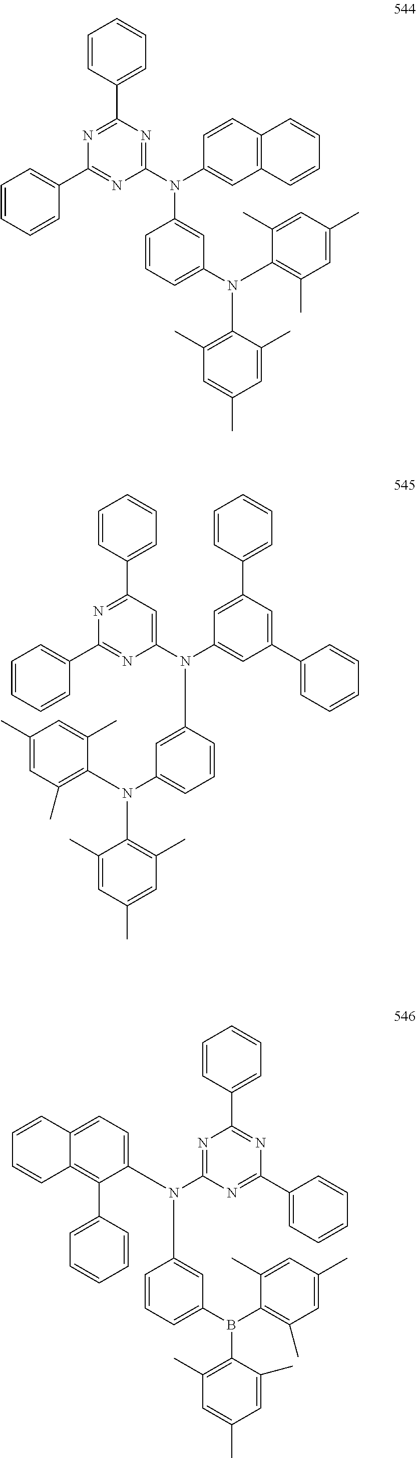 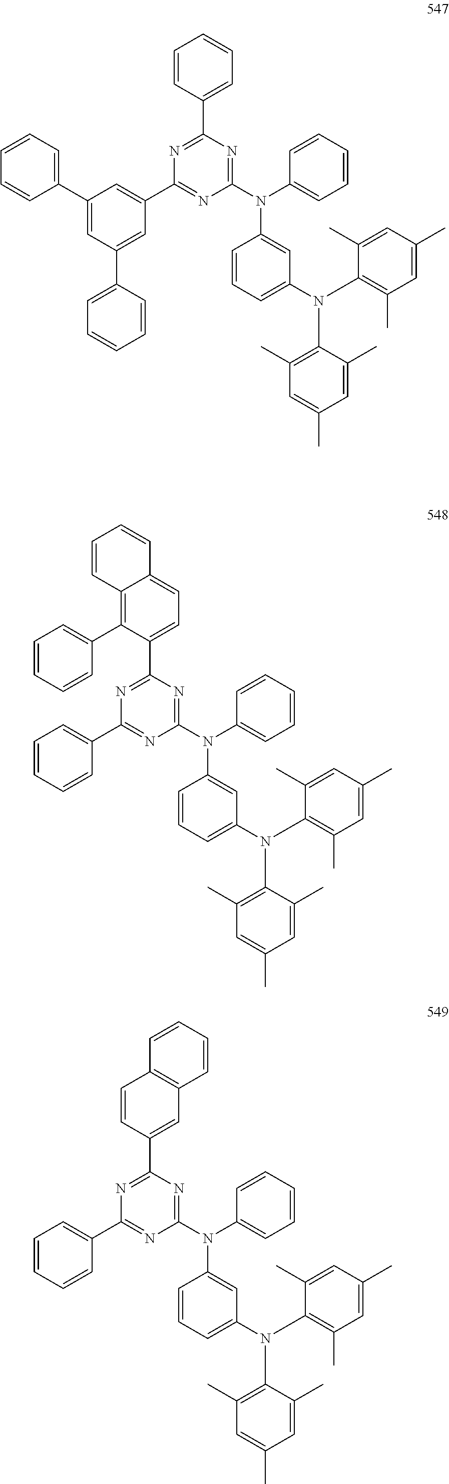 [SMILES: Cc1cc(C)c(N(c2cccc(N(c3ccccc3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4-c4ccccc4)n3)c2)c2c(C)cc(C)cc2C)c(C)c1 ; Cc1cc(C)c(N(c2cccc(N(c3ccccc3)c3nc(-c4ccccc4)nc(-c4ccc5ccccc5c4)n3)c2)c2c(C)cc(C)cc2C)c(C)c1] 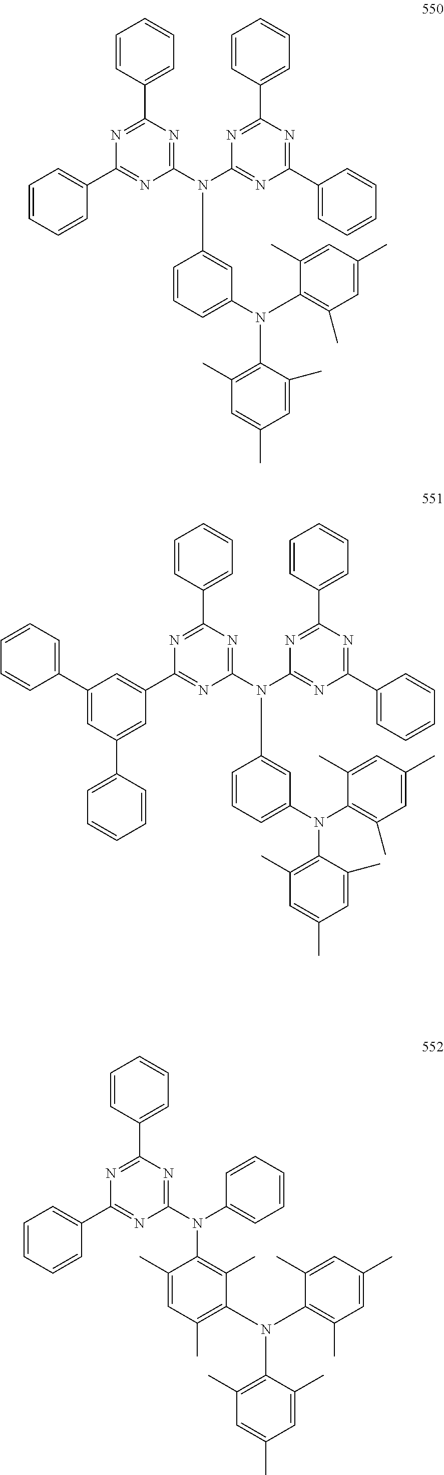 [SMILES: Cc1cc(C)c(N(c2cccc(N(c3nc(-c4ccccc4)nc(-c4ccccc4)n3)c3nc(-c4ccccc4)nc(-c4ccccc4)n3)c2)c2c(C)cc(C)cc2C)c(C)c1 ; Cc1cc(C)c(N(c2cccc(N(c3nc(-c4ccccc4)nc(-c4ccccc4)n3)c3nc(-c4ccccc4)nc(-c4cc(-c5ccccc5)cc(-c5ccccc5)c4)n3)c2)c2c(C)cc(C)cc2C)c(C)c1 ; Cc1cc(C)c(N(c2c(C)cc(C)cc2C)c2c(C)cc(C)c(N(c3ccccc3)c3nc(-c4ccccc4)nc(-c4ccccc4)n3)c2C)c(C)c1] 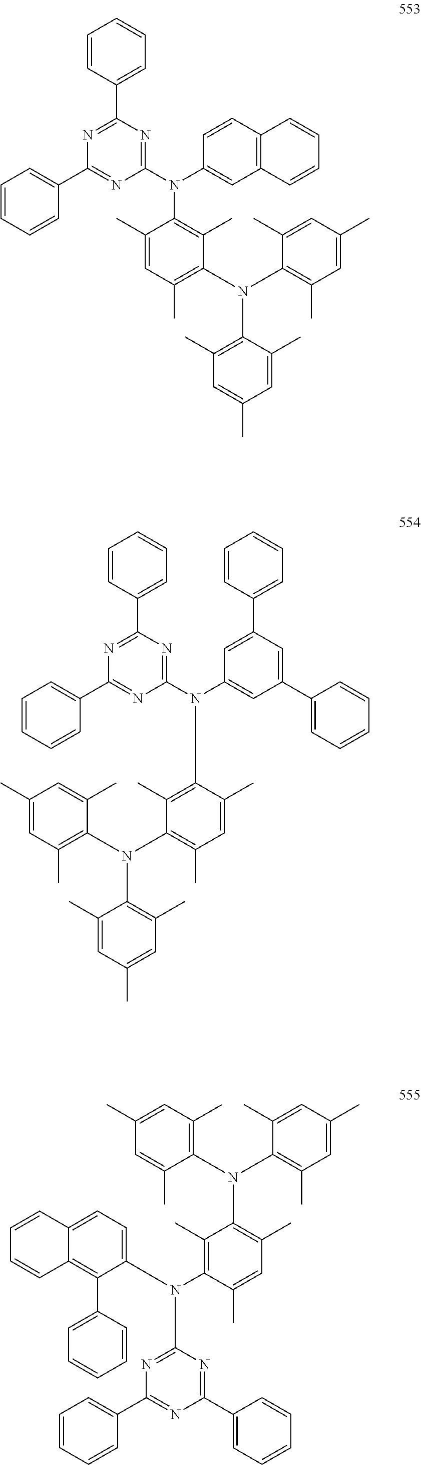 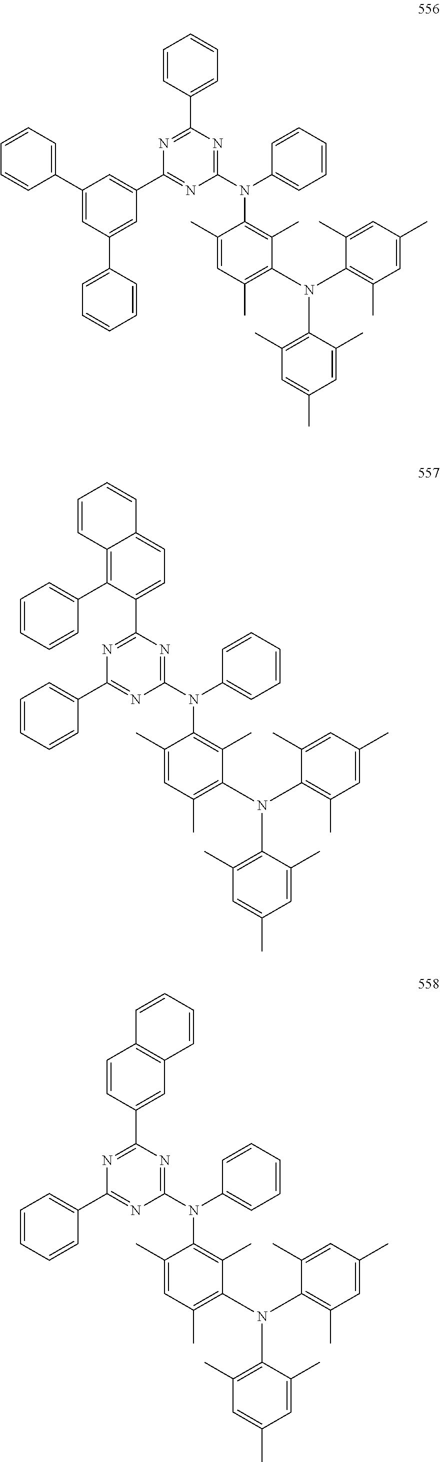 [SMILES: Cc1cc(C)c(N(c2c(C)cc(C)cc2C)c2c(C)cc(C)c(N(c3ccccc3)c3nc(-c4ccccc4)nc(-c4cc(-c5ccccc5)cc(-c5ccccc5)c4)n3)c2C)c(C)c1 ; Cc1cc(C)c(N(c2c(C)cc(C)cc2C)c2c(C)cc(C)c(N(c3ccccc3)c3nc(-c4ccccc4)nc(-c4ccc5ccccc5c4-c4ccccc4)n3)c2C)c(C)c1 ; Cc1cc(C)c(N(c2c(C)cc(C)cc2C)c2c(C)cc(C)c(N(c3ccccc3)c3nc(-c4ccccc4)nc(-c4ccc5ccccc5c4)n3)c2C)c(C)c1] 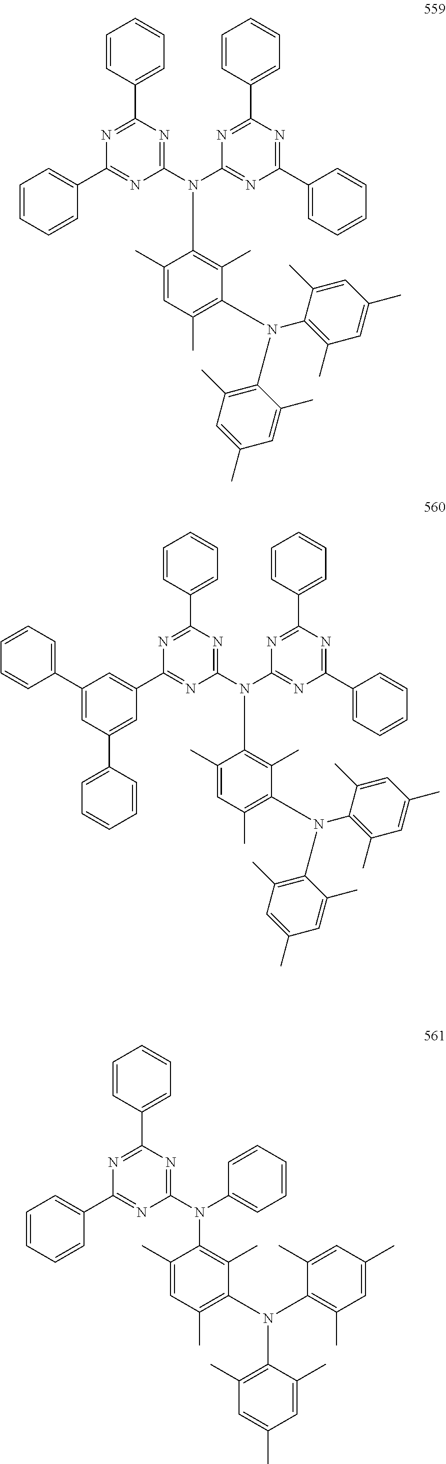 [SMILES: Cc1cc(C)c(N(c2c(C)cc(C)cc2C)c2c(C)cc(C)c(N(c3nc(-c4ccccc4)nc(-c4ccccc4)n3)c3nc(-c4ccccc4)nc(-c4ccccc4)n3)c2C)c(C)c1 ; Cc1cc(C)c(N(c2c(C)cc(C)cc2C)c2c(C)cc(C)c(N(c3nc(-c4ccccc4)nc(-c4ccccc4)n3)c3nc(-c4ccccc4)nc(-c4cc(-c5ccccc5)cc(-c5ccccc5)c4)n3)c2C)c(C)c1 ; Cc1cc(C)c(N(c2c(C)cc(C)cc2C)c2c(C)cc(C)c(N(c3ccccc3)c3nc(-c4ccccc4)nc(-c4ccccc4)n3)c2C)c(C)c1] 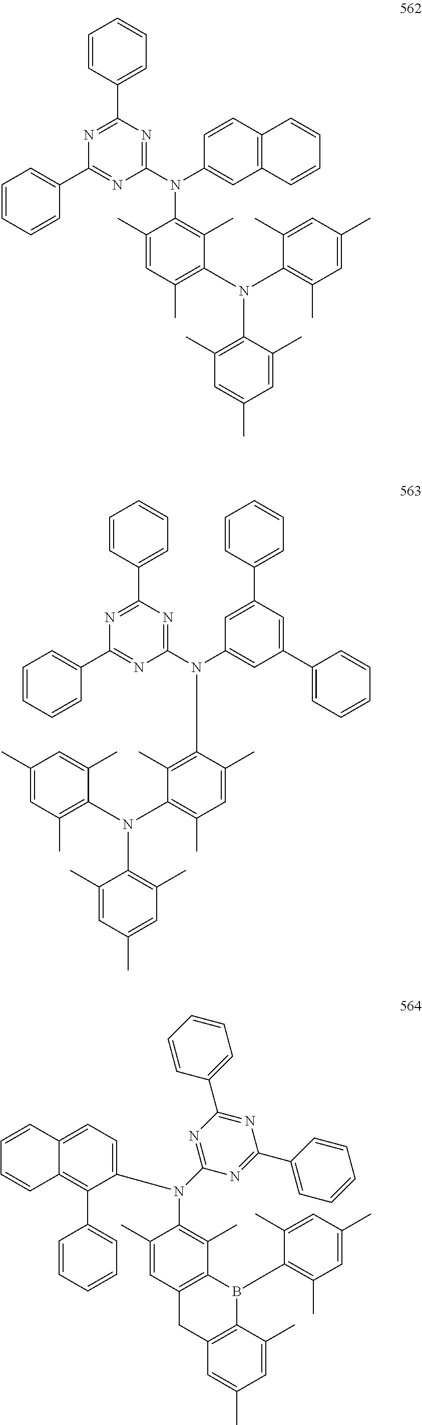 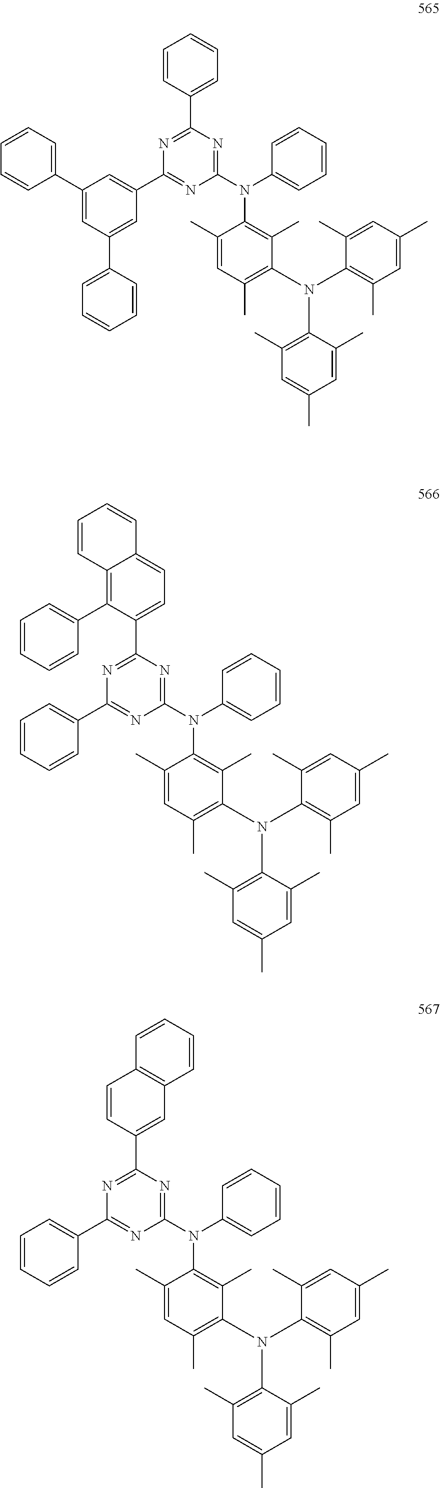 [SMILES: Cc1cc(C)c(N(c2c(C)cc(C)cc2C)c2c(C)cc(C)c(N(c3ccccc3)c3nc(-c4ccccc4)nc(-c4cc(-c5ccccc5)cc(-c5ccccc5)c4)n3)c2C)c(C)c1 ; Cc1cc(C)c(N(c2c(C)cc(C)cc2C)c2c(C)cc(C)c(N(c3ccccc3)c3nc(-c4ccccc4)nc(-c4ccc5ccccc5c4-c4ccccc4)n3)c2C)c(C)c1 ; Cc1cc(C)c(N(c2c(C)cc(C)cc2C)c2c(C)cc(C)c(N(c3ccccc3)c3nc(-c4ccccc4)nc(-c4ccc5ccccc5c4)n3)c2C)c(C)c1] 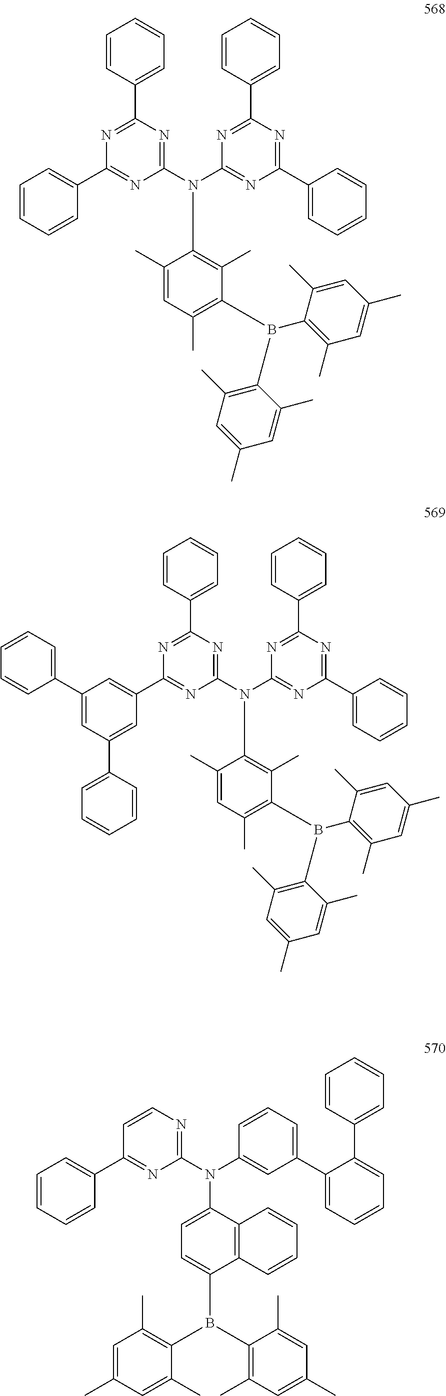 [SMILES: Cc1cc(C)c(B(c2c(C)cc(C)cc2C)c2c(C)cc(C)c(N(c3nc(-c4ccccc4)nc(-c4ccccc4)n3)c3nc(-c4ccccc4)nc(-c4ccccc4)n3)c2C)c(C)c1 ; Cc1cc(C)c(B(c2c(C)cc(C)cc2C)c2c(C)cc(C)c(N(c3nc(-c4ccccc4)nc(-c4ccccc4)n3)c3nc(-c4ccccc4)nc(-c4cc(-c5ccccc5)cc(-c5ccccc5)c4)n3)c2C)c(C)c1 ; Cc1cc(C)c(B(c2c(C)cc(C)cc2C)c2ccc(N(c3cccc(-c4ccccc4-c4ccccc4)c3)c3nccc(-c4ccccc4)n3)c3ccccc23)c(C)c1] 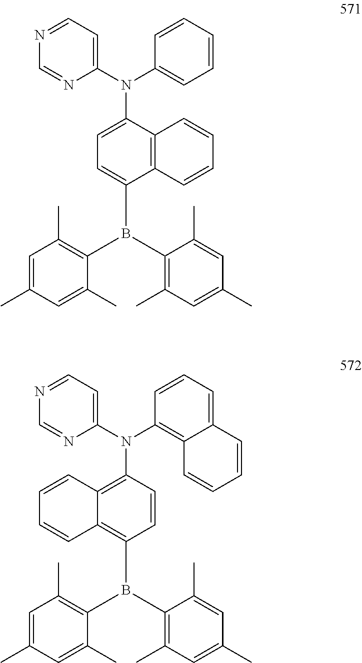 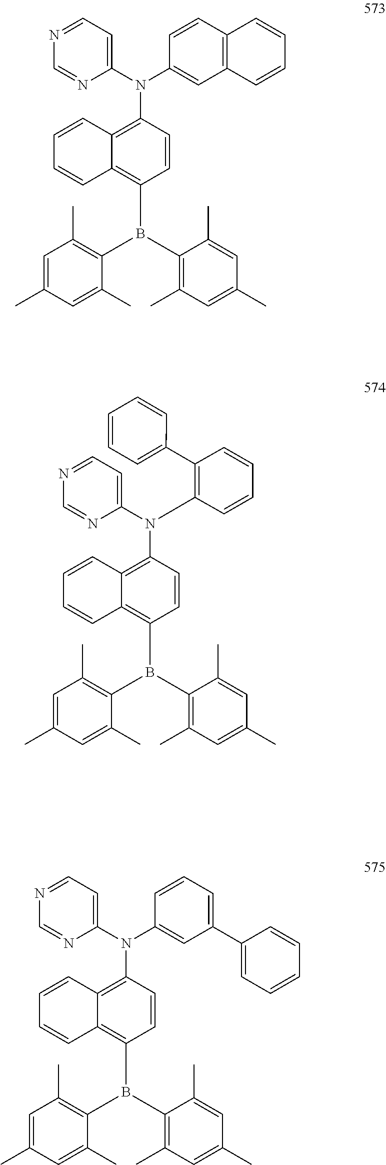 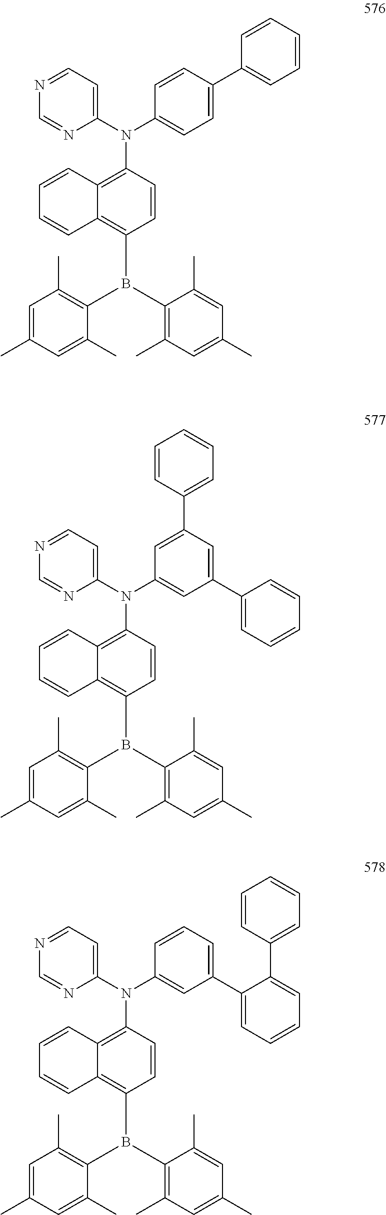 [SMILES: Cc1cc(C)c(B(c2c(C)cc(C)cc2C)c2ccc(N(c3ccc(-c4ccccc4)cc3)c3ccncn3)c3ccccc23)c(C)c1 ; Cc1cc(C)c(B(c2c(C)cc(C)cc2C)c2ccc(N(c3cc(-c4ccccc4)cc(-c4ccccc4)c3)c3ccncn3)c3ccccc23)c(C)c1 ; Cc1cc(C)c(B(c2c(C)cc(C)cc2C)c2ccc(N(c3cccc(-c4ccccc4-c4ccccc4)c3)c3ccncn3)c3ccccc23)c(C)c1] 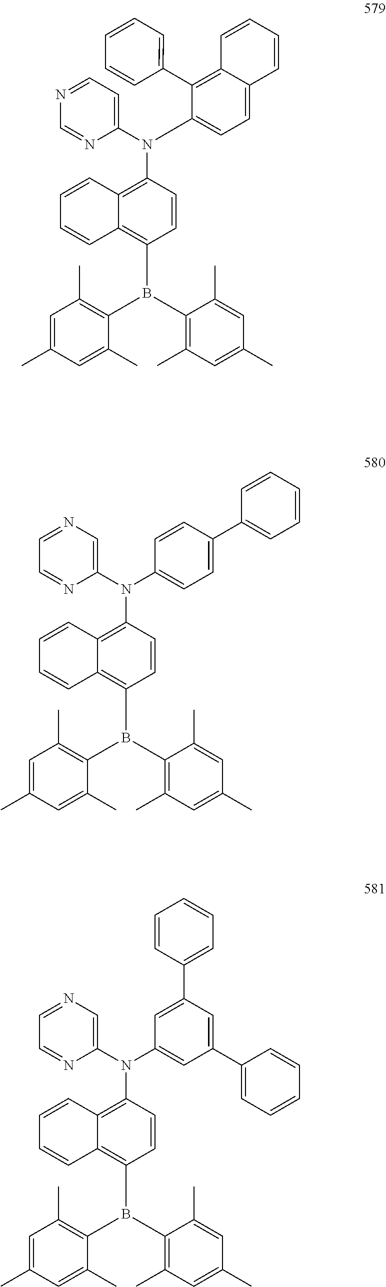 [SMILES: Cc1cc(C)c(B(c2c(C)cc(C)cc2C)c2ccc(N(c3ccncn3)c3ccc4ccccc4c3-c3ccccc3)c3ccccc23)c(C)c1 ; Cc1cc(C)c(B(c2c(C)cc(C)cc2C)c2ccc(N(c3ccc(-c4ccccc4)cc3)c3cnccn3)c3ccccc23)c(C)c1 ; Cc1cc(C)c(B(c2c(C)cc(C)cc2C)c2ccc(N(c3cc(-c4ccccc4)cc(-c4ccccc4)c3)c3cnccn3)c3ccccc23)c(C)c1] 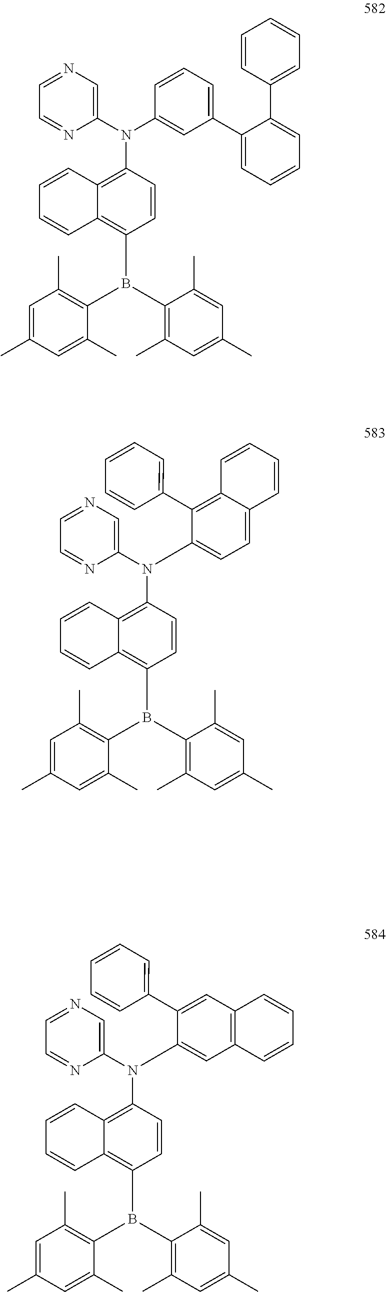 [SMILES: Cc1cc(C)c(B(c2c(C)cc(C)cc2C)c2ccc(N(c3cccc(-c4ccccc4-c4ccccc4)c3)c3cnccn3)c3ccccc23)c(C)c1 ; Cc1cc(C)c(B(c2c(C)cc(C)cc2C)c2ccc(N(c3cnccn3)c3ccc4ccccc4c3-c3ccccc3)c3ccccc23)c(C)c1 ; Cc1cc(C)c(B(c2c(C)cc(C)cc2C)c2ccc(N(c3cnccn3)c3cc4ccccc4cc3-c3ccccc3)c3ccccc23)c(C)c1] 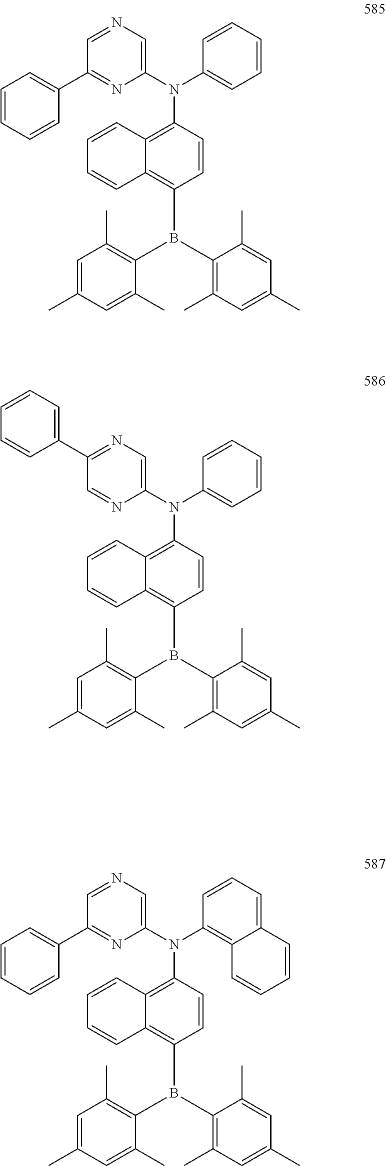 [SMILES: Cc1cc(C)c(B(c2c(C)cc(C)cc2C)c2ccc(N(c3ccccc3)c3cncc(-c4ccccc4)n3)c3ccccc23)c(C)c1 ; Cc1cc(C)c(B(c2c(C)cc(C)cc2C)c2ccc(N(c3ccccc3)c3cnc(-c4ccccc4)cn3)c3ccccc23)c(C)c1 ; Cc1cc(C)c(B(c2c(C)cc(C)cc2C)c2ccc(N(c3cncc(-c4ccccc4)n3)c3cccc4ccccc34)c3ccccc23)c(C)c1] 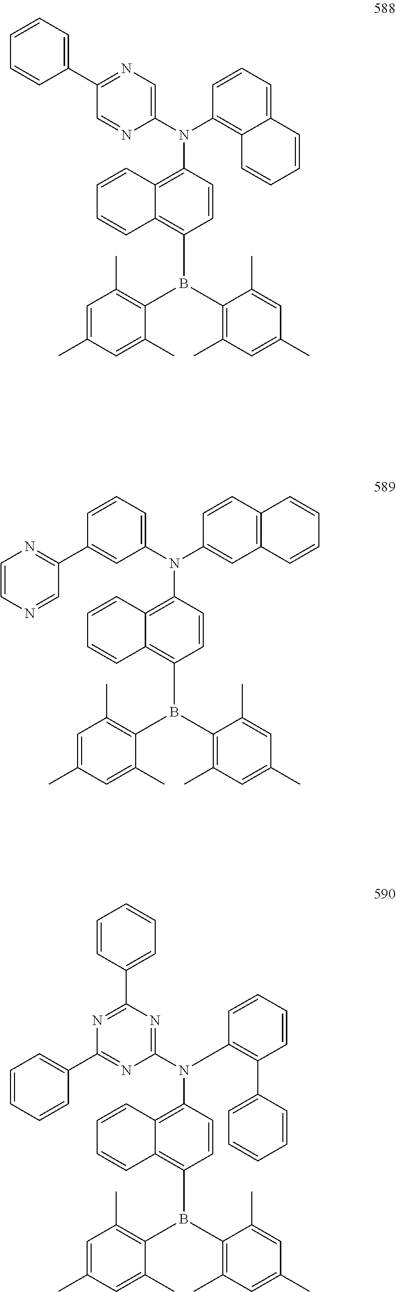 [SMILES: Cc1cc(C)c(B(c2c(C)cc(C)cc2C)c2ccc(N(c3cnc(-c4ccccc4)cn3)c3cccc4ccccc34)c3ccccc23)c(C)c1 ; Cc1cc(C)c(B(c2c(C)cc(C)cc2C)c2ccc(N(c3cccc(-c4cnccn4)c3)c3ccc4ccccc4c3)c3ccccc23)c(C)c1 ; Cc1cc(C)c(B(c2c(C)cc(C)cc2C)c2ccc(N(c3nc(-c4ccccc4)nc(-c4ccccc4)n3)c3ccccc3-c3ccccc3)c3ccccc23)c(C)c1] 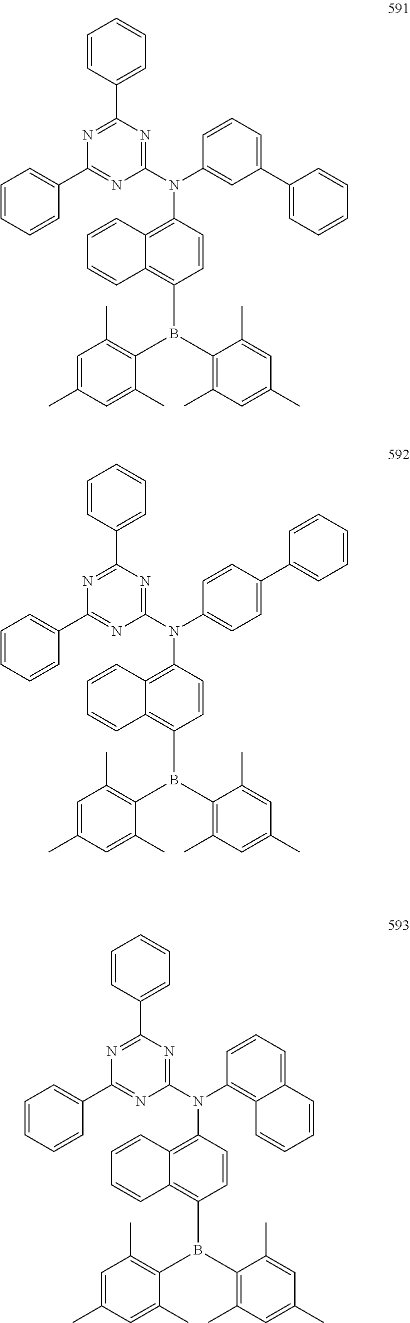 [SMILES: Cc1cc(C)c(B(c2c(C)cc(C)cc2C)c2ccc(N(c3cccc(-c4ccccc4)c3)c3nc(-c4ccccc4)nc(-c4ccccc4)n3)c3ccccc23)c(C)c1 ; Cc1cc(C)c(B(c2c(C)cc(C)cc2C)c2ccc(N(c3ccc(-c4ccccc4)cc3)c3nc(-c4ccccc4)nc(-c4ccccc4)n3)c3ccccc23)c(C)c1 ; Cc1cc(C)c(B(c2c(C)cc(C)cc2C)c2ccc(N(c3nc(-c4ccccc4)nc(-c4ccccc4)n3)c3cccc4ccccc34)c3ccccc23)c(C)c1] 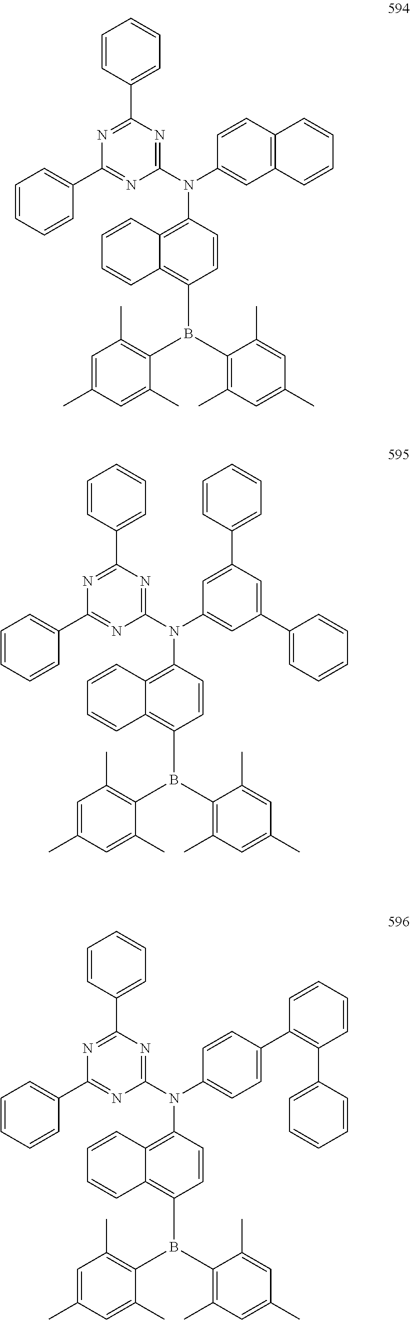 [SMILES: Cc1cc(C)c(B(c2c(C)cc(C)cc2C)c2ccc(N(c3ccc4ccccc4c3)c3nc(-c4ccccc4)nc(-c4ccccc4)n3)c3ccccc23)c(C)c1 ; Cc1cc(C)c(B(c2c(C)cc(C)cc2C)c2ccc(N(c3cc(-c4ccccc4)cc(-c4ccccc4)c3)c3nc(-c4ccccc4)nc(-c4ccccc4)n3)c3ccccc23)c(C)c1 ; Cc1cc(C)c(B(c2c(C)cc(C)cc2C)c2ccc(N(c3ccc(-c4ccccc4-c4ccccc4)cc3)c3nc(-c4ccccc4)nc(-c4ccccc4)n3)c3ccccc23)c(C)c1] 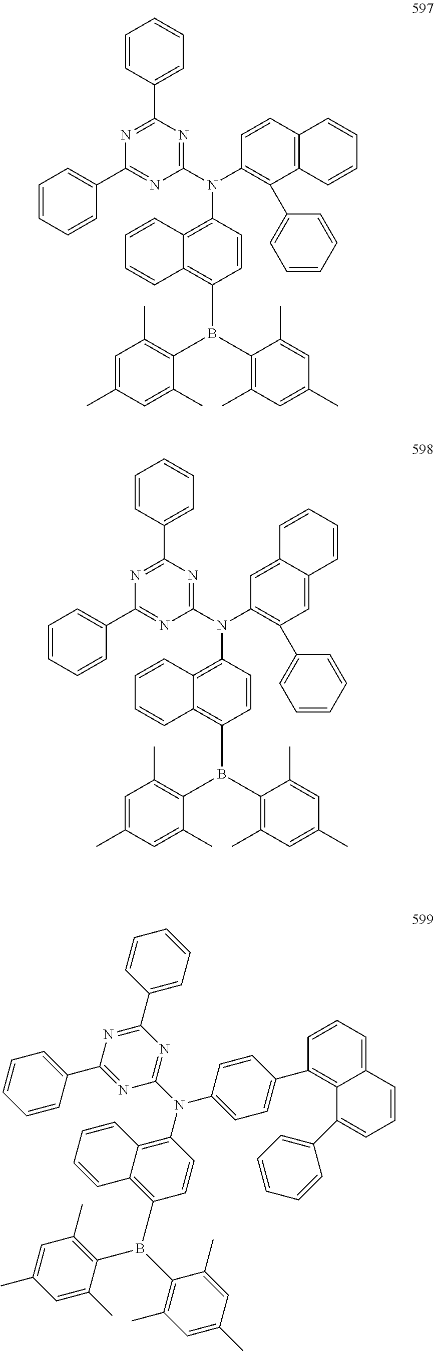 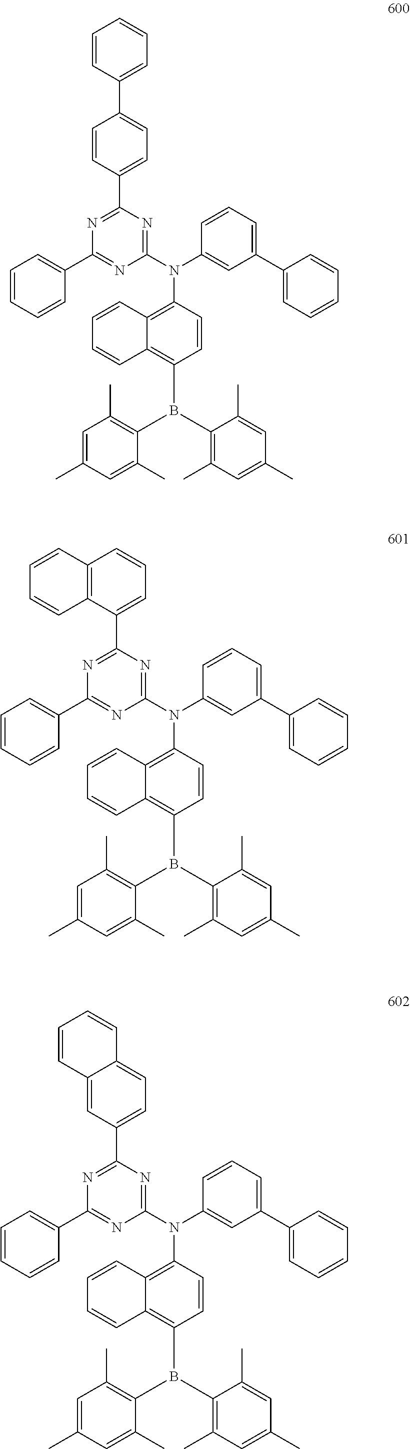 [SMILES: Cc1cc(C)c(B(c2c(C)cc(C)cc2C)c2ccc(N(c3cccc(-c4ccccc4)c3)c3nc(-c4ccccc4)nc(-c4ccc(-c5ccccc5)cc4)n3)c3ccccc23)c(C)c1 ; Cc1cc(C)c(B(c2c(C)cc(C)cc2C)c2ccc(N(c3cccc(-c4ccccc4)c3)c3nc(-c4ccccc4)nc(-c4cccc5ccccc45)n3)c3ccccc23)c(C)c1 ; Cc1cc(C)c(B(c2c(C)cc(C)cc2C)c2ccc(N(c3cccc(-c4ccccc4)c3)c3nc(-c4ccccc4)nc(-c4ccc5ccccc5c4)n3)c3ccccc23)c(C)c1] 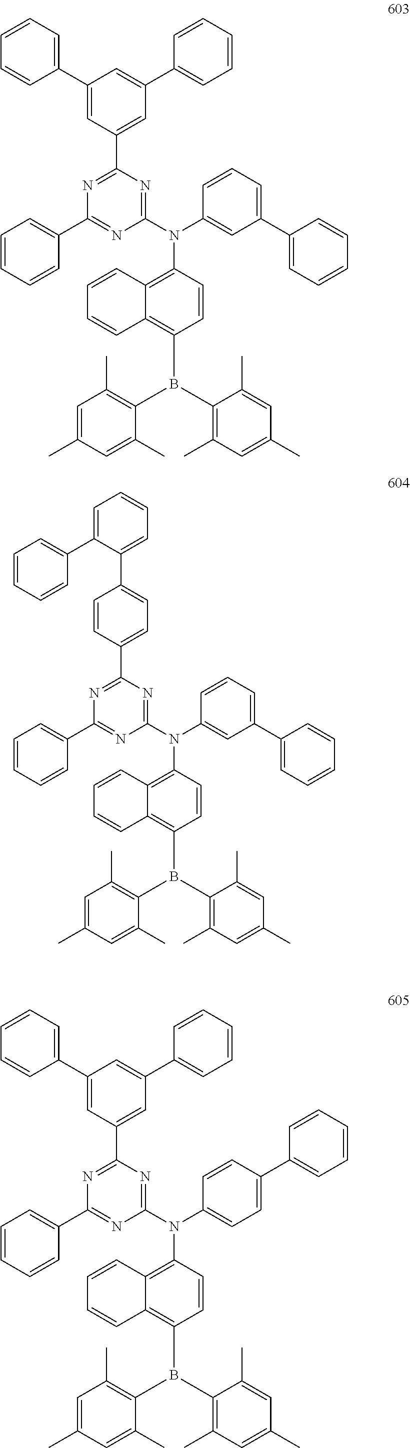 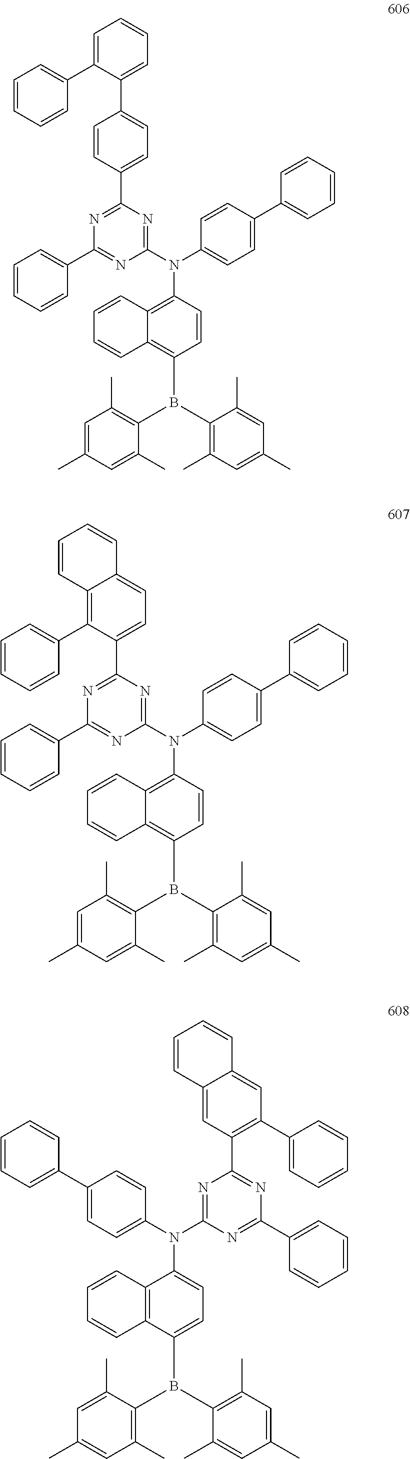 [SMILES: Cc1cc(C)c(B(c2c(C)cc(C)cc2C)c2ccc(N(c3ccc(-c4ccccc4)cc3)c3nc(-c4ccccc4)nc(-c4ccc(-c5ccccc5-c5ccccc5)cc4)n3)c3ccccc23)c(C)c1 ; Cc1cc(C)c(B(c2c(C)cc(C)cc2C)c2ccc(N(c3ccc(-c4ccccc4)cc3)c3nc(-c4ccccc4)nc(-c4ccc5ccccc5c4-c4ccccc4)n3)c3ccccc23)c(C)c1 ; Cc1cc(C)c(B(c2c(C)cc(C)cc2C)c2ccc(N(c3ccc(-c4ccccc4)cc3)c3nc(-c4ccccc4)nc(-c4cc5ccccc5cc4-c4ccccc4)n3)c3ccccc23)c(C)c1] 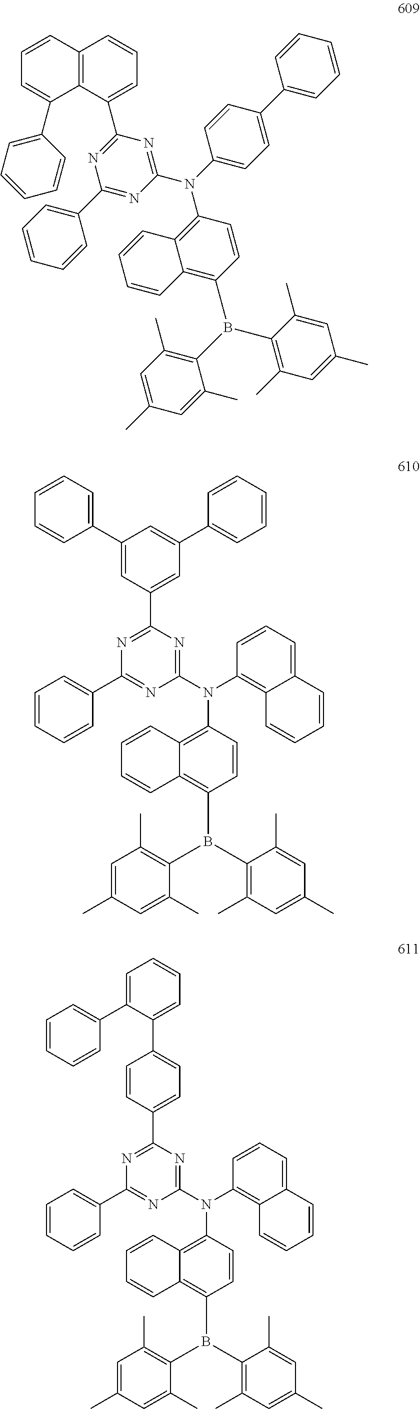 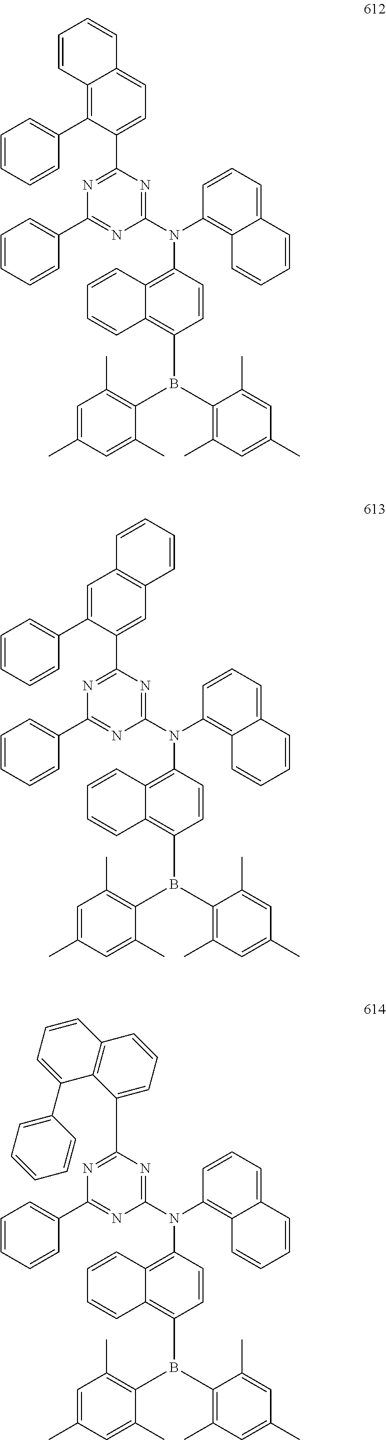 [SMILES: Cc1cc(C)c(B(c2c(C)cc(C)cc2C)c2ccc(N(c3nc(-c4ccccc4)nc(-c4ccc5ccccc5c4-c4ccccc4)n3)c3cccc4ccccc34)c3ccccc23)c(C)c1 ; Cc1cc(C)c(B(c2c(C)cc(C)cc2C)c2ccc(N(c3nc(-c4ccccc4)nc(-c4cc5ccccc5cc4-c4ccccc4)n3)c3cccc4ccccc34)c3ccccc23)c(C)c1 ; Cc1cc(C)c(B(c2c(C)cc(C)cc2C)c2ccc(N(c3nc(-c4ccccc4)nc(-c4cccc5cccc(-c6ccccc6)c45)n3)c3cccc4ccccc34)c3ccccc23)c(C)c1] 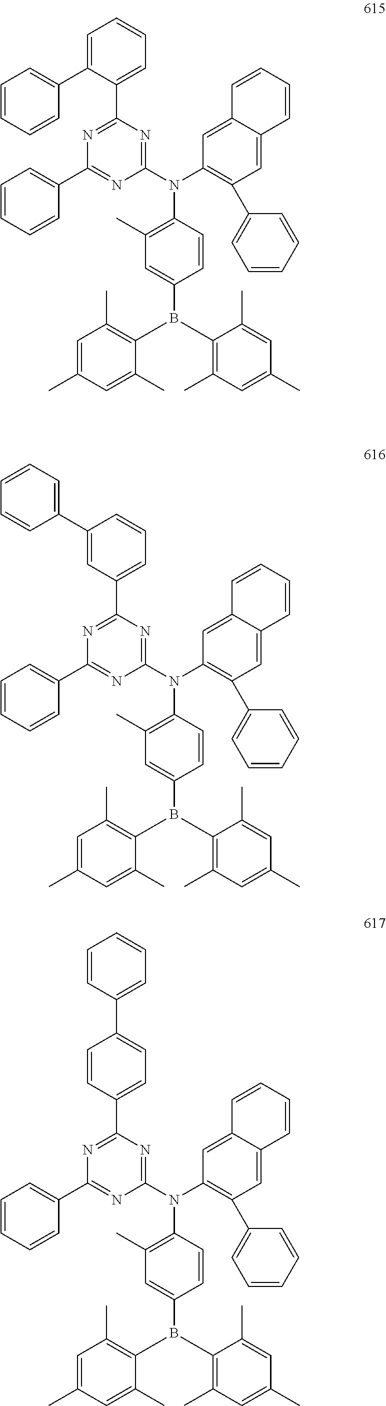 [SMILES: Cc1cc(C)c(B(c2ccc(N(c3nc(-c4ccccc4)nc(-c4ccccc4-c4ccccc4)n3)c3cc4ccccc4cc3-c3ccccc3)c(C)c2)c2c(C)cc(C)cc2C)c(C)c1 ; Cc1cc(C)c(B(c2ccc(N(c3nc(-c4ccccc4)nc(-c4cccc(-c5ccccc5)c4)n3)c3cc4ccccc4cc3-c3ccccc3)c(C)c2)c2c(C)cc(C)cc2C)c(C)c1 ; Cc1cc(C)c(B(c2ccc(N(c3nc(-c4ccccc4)nc(-c4ccc(-c5ccccc5)cc4)n3)c3cc4ccccc4cc3-c3ccccc3)c(C)c2)c2c(C)cc(C)cc2C)c(C)c1] 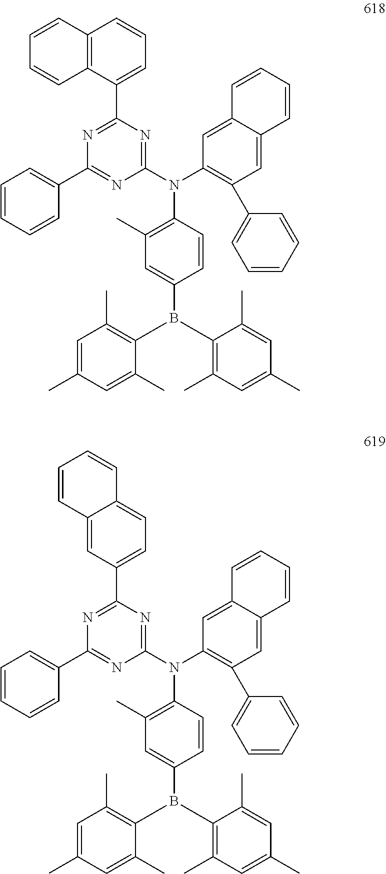 [SMILES: Cc1cc(C)c(B(c2ccc(N(c3nc(-c4ccccc4)nc(-c4cccc5ccccc45)n3)c3cc4ccccc4cc3-c3ccccc3)c(C)c2)c2c(C)cc(C)cc2C)c(C)c1 ; Cc1cc(C)c(B(c2ccc(N(c3nc(-c4ccccc4)nc(-c4ccc5ccccc5c4)n3)c3cc4ccccc4cc3-c3ccccc3)c(C)c2)c2c(C)cc(C)cc2C)c(C)c1] 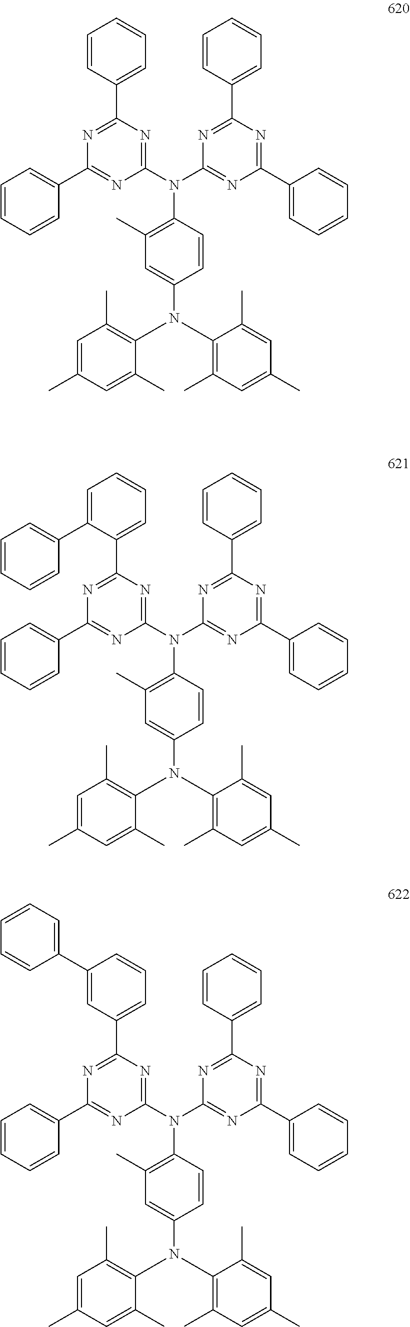 [SMILES: Cc1cc(C)c(N(c2ccc(N(c3nc(-c4ccccc4)nc(-c4ccccc4)n3)c3nc(-c4ccccc4)nc(-c4ccccc4)n3)c(C)c2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccccc4-c4ccccc4)n3)c(C)c2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4cccc(-c5ccccc5)c4)n3)c(C)c2)c2c(C)cc(C)cc2C)c(C)c1] 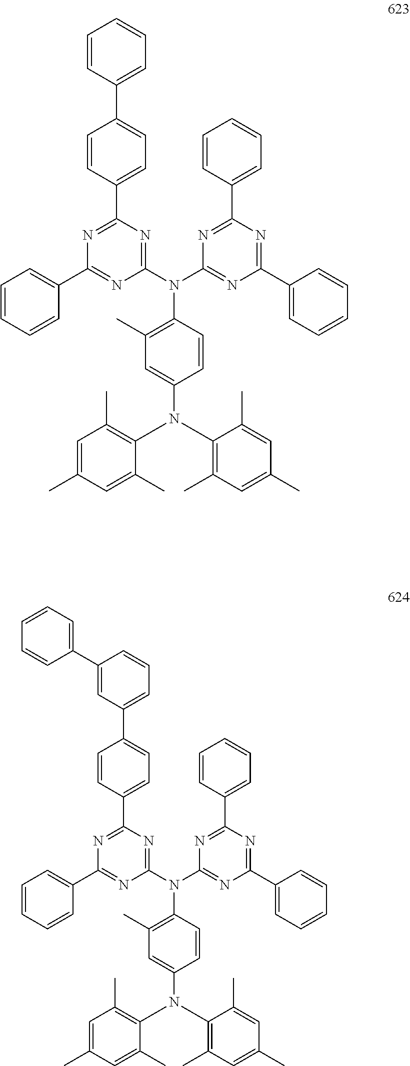 [SMILES: Cc1cc(C)c(N(c2ccc(N(c3nc(-c4ccccc4)nc(-c4ccccc4)n3)c3nc(-c4ccccc4)nc(-c4ccc(-c5ccccc5)cc4)n3)c(C)c2)c2c(C)cc(C)cc2C)c(C)c1 ; Cc1cc(C)c(N(c2ccc(N(c3nc(-c4ccccc4)nc(-c4ccccc4)n3)c3nc(-c4ccccc4)nc(-c4ccc(-c5cccc(-c6ccccc6)c5)cc4)n3)c(C)c2)c2c(C)cc(C)cc2C)c(C)c1] 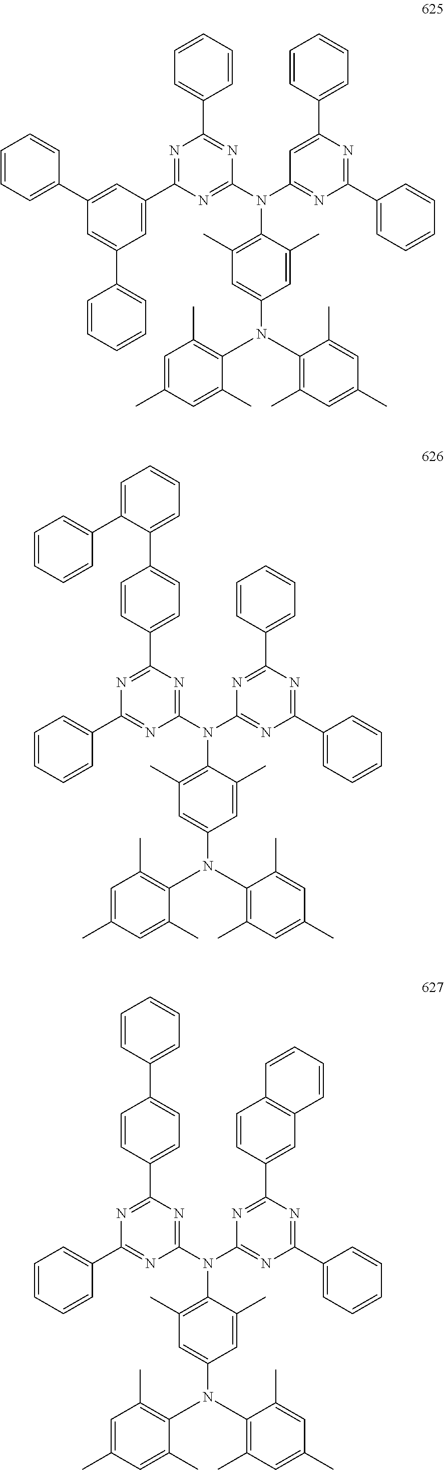 [SMILES: Cc1cc(C)c(N(c2cc(C)c(N(c3cc(-c4ccccc4)nc(-c4ccccc4)n3)c3nc(-c4ccccc4)nc(-c4cc(-c5ccccc5)cc(-c5ccccc5)c4)n3)c(C)c2)c2c(C)cc(C)cc2C)c(C)c1 ; Cc1cc(C)c(N(c2cc(C)c(N(c3nc(-c4ccccc4)nc(-c4ccccc4)n3)c3nc(-c4ccccc4)nc(-c4ccc(-c5ccccc5-c5ccccc5)cc4)n3)c(C)c2)c2c(C)cc(C)cc2C)c(C)c1 ; Cc1cc(C)c(N(c2cc(C)c(N(c3nc(-c4ccccc4)nc(-c4ccc(-c5ccccc5)cc4)n3)c3nc(-c4ccccc4)nc(-c4ccc5ccccc5c4)n3)c(C)c2)c2c(C)cc(C)cc2C)c(C)c1] 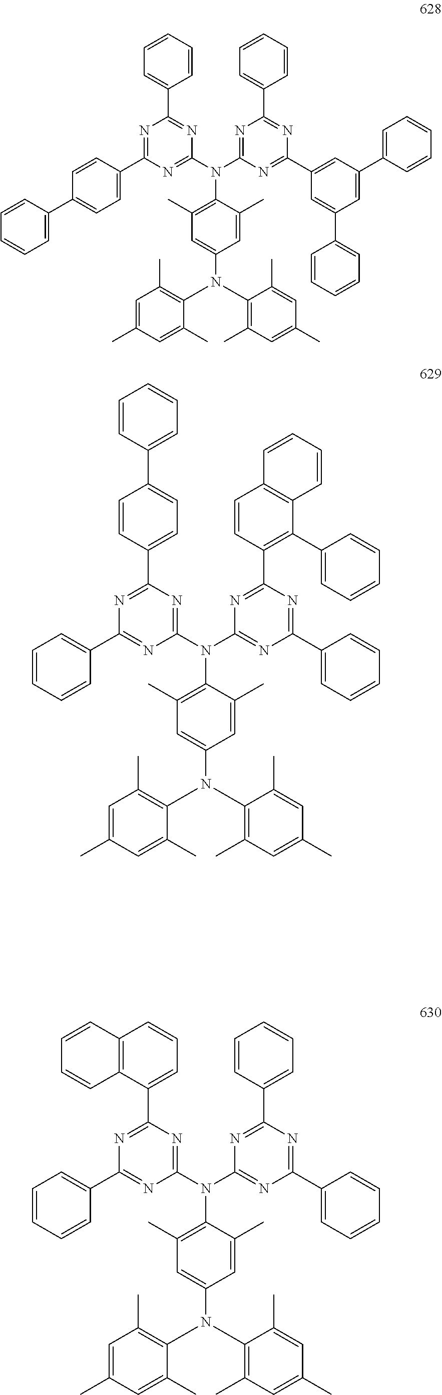 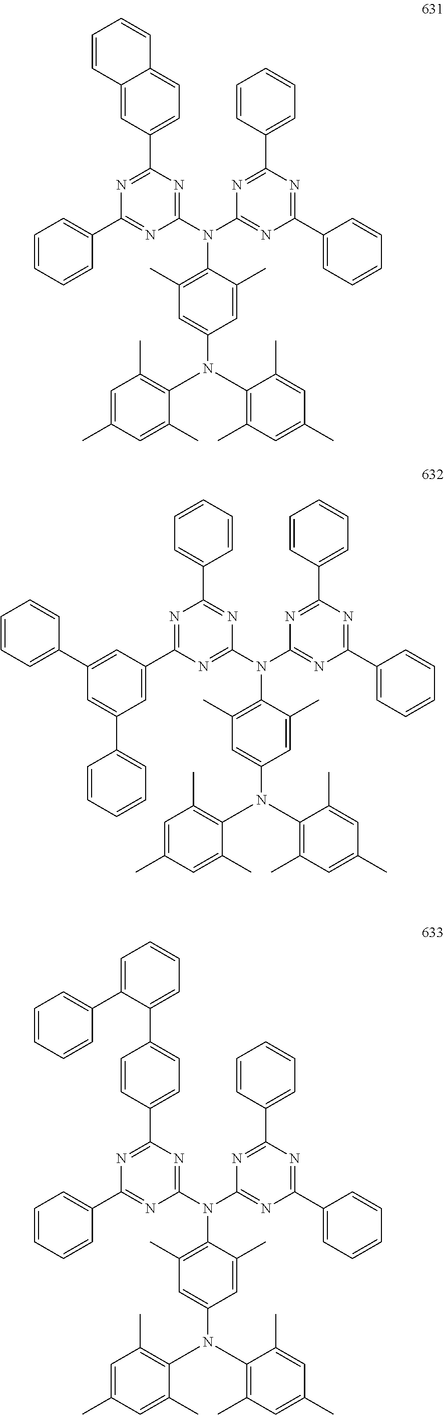 [SMILES: Cc1cc(C)c(N(c2cc(C)c(N(c3nc(-c4ccccc4)nc(-c4ccccc4)n3)c3nc(-c4ccccc4)nc(-c4ccc5ccccc5c4)n3)c(C)c2)c2c(C)cc(C)cc2C)c(C)c1 ; Cc1cc(C)c(N(c2cc(C)c(N(c3nc(-c4ccccc4)nc(-c4ccccc4)n3)c3nc(-c4ccccc4)nc(-c4cc(-c5ccccc5)cc(-c5ccccc5)c4)n3)c(C)c2)c2c(C)cc(C)cc2C)c(C)c1 ; Cc1cc(C)c(N(c2cc(C)c(N(c3nc(-c4ccccc4)nc(-c4ccccc4)n3)c3nc(-c4ccccc4)nc(-c4ccc(-c5ccccc5-c5ccccc5)cc4)n3)c(C)c2)c2c(C)cc(C)cc2C)c(C)c1] 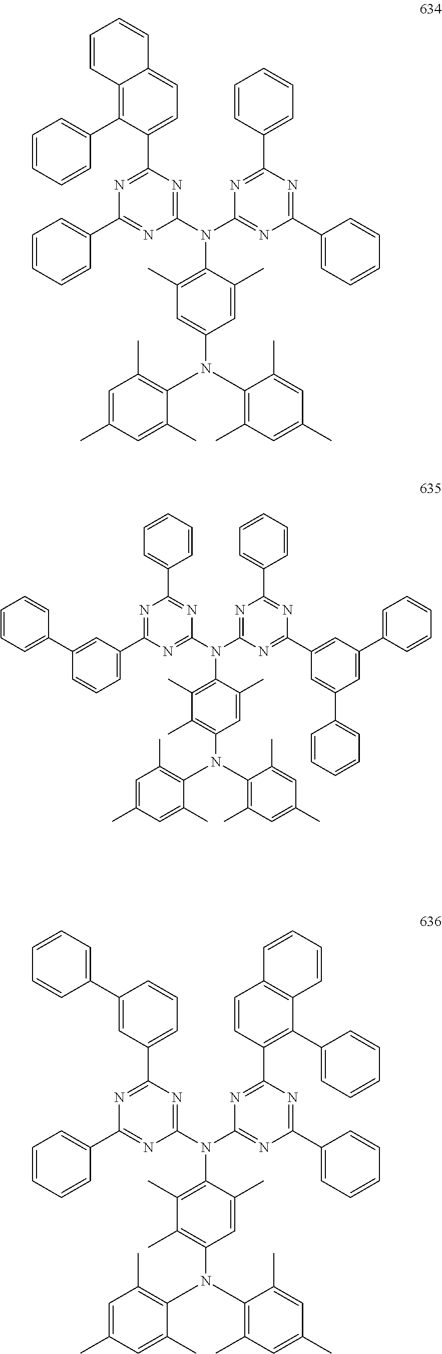 [SMILES: Cc1cc(C)c(N(c2cc(C)c(N(c3nc(-c4ccccc4)nc(-c4ccccc4)n3)c3nc(-c4ccccc4)nc(-c4ccc5ccccc5c4-c4ccccc4)n3)c(C)c2)c2c(C)cc(C)cc2C)c(C)c1 ; Cc1cc(C)c(N(c2cc(C)c(N(c3nc(-c4ccccc4)nc(-c4cccc(-c5ccccc5)c4)n3)c3nc(-c4ccccc4)nc(-c4cc(-c5ccccc5)cc(-c5ccccc5)c4)n3)c(C)c2C)c2c(C)cc(C)cc2C)c(C)c1 ; Cc1cc(C)c(N(c2cc(C)c(N(c3nc(-c4ccccc4)nc(-c4cccc(-c5ccccc5)c4)n3)c3nc(-c4ccccc4)nc(-c4ccc5ccccc5c4-c4ccccc4)n3)c(C)c2C)c2c(C)cc(C)cc2C)c(C)c1] 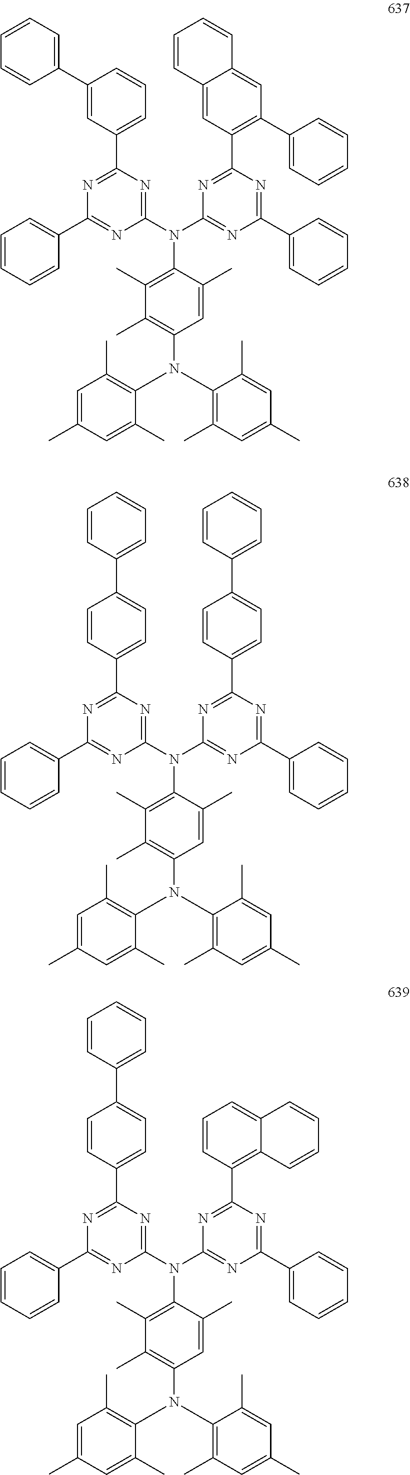 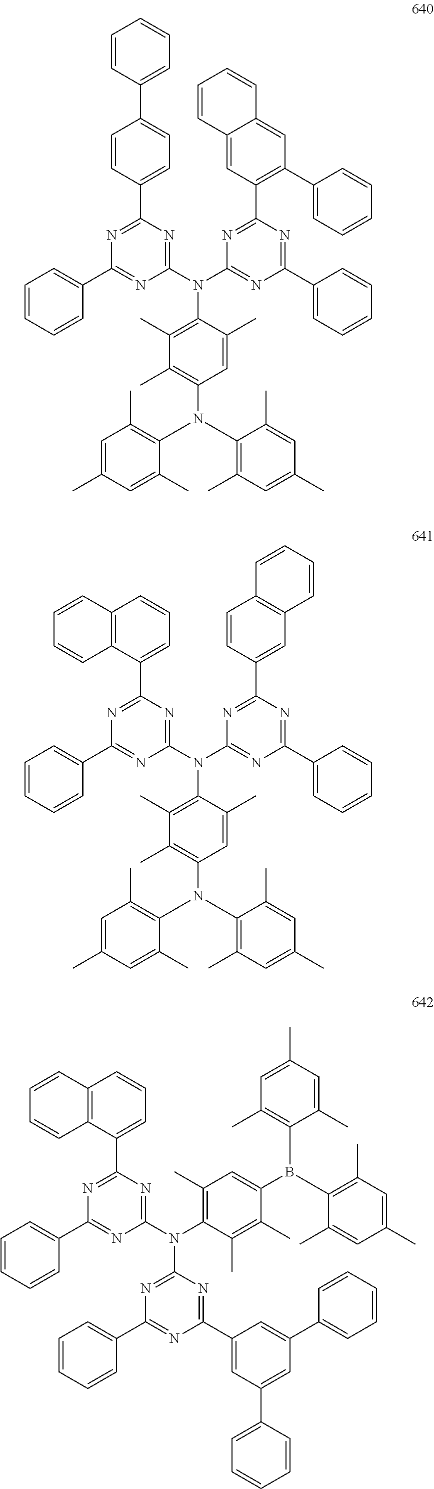 [SMILES: Cc1cc(C)c(N(c2cc(C)c(N(c3nc(-c4ccccc4)nc(-c4ccc(-c5ccccc5)cc4)n3)c3nc(-c4ccccc4)nc(-c4cc5ccccc5cc4-c4ccccc4)n3)c(C)c2C)c2c(C)cc(C)cc2C)c(C)c1 ; Cc1cc(C)c(N(c2cc(C)c(N(c3nc(-c4ccccc4)nc(-c4ccc5ccccc5c4)n3)c3nc(-c4ccccc4)nc(-c4cccc5ccccc45)n3)c(C)c2C)c2c(C)cc(C)cc2C)c(C)c1 ; Cc1cc(C)c(B(c2cc(C)c(N(c3nc(-c4ccccc4)nc(-c4cc(-c5ccccc5)cc(-c5ccccc5)c4)n3)c3nc(-c4ccccc4)nc(-c4cccc5ccccc45)n3)c(C)c2C)c2c(C)cc(C)cc2C)c(C)c1] 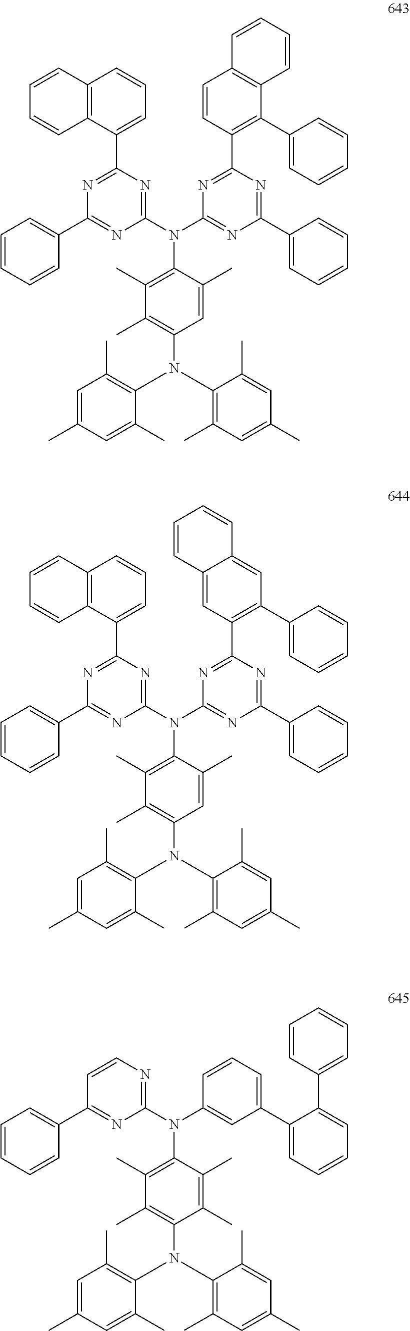 [SMILES: Cc1cc(C)c(N(c2cc(C)c(N(c3nc(-c4ccccc4)nc(-c4ccc5ccccc5c4-c4ccccc4)n3)c3nc(-c4ccccc4)nc(-c4cccc5ccccc45)n3)c(C)c2C)c2c(C)cc(C)cc2C)c(C)c1 ; Cc1cc(C)c(N(c2cc(C)c(N(c3nc(-c4ccccc4)nc(-c4cc5ccccc5cc4-c4ccccc4)n3)c3nc(-c4ccccc4)nc(-c4cccc5ccccc45)n3)c(C)c2C)c2c(C)cc(C)cc2C)c(C)c1 ; Cc1cc(C)c(N(c2c(C)cc(C)cc2C)c2c(C)c(C)c(N(c3cccc(-c4ccccc4-c4ccccc4)c3)c3nccc(-c4ccccc4)n3)c(C)c2C)c(C)c1] 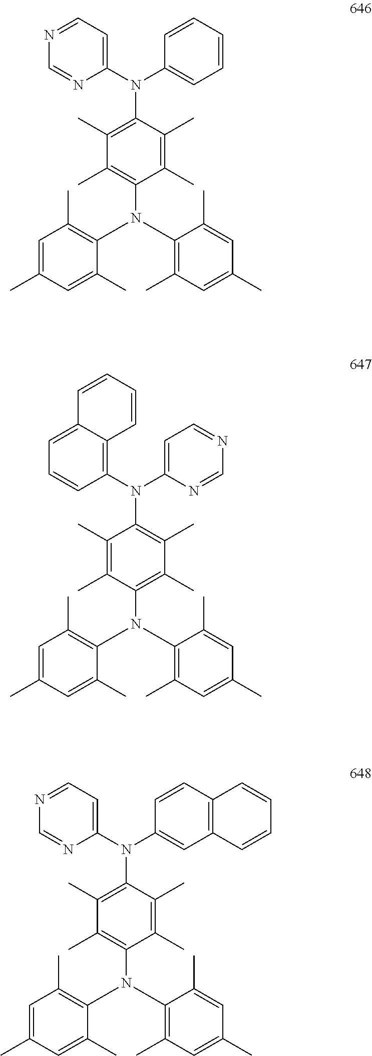 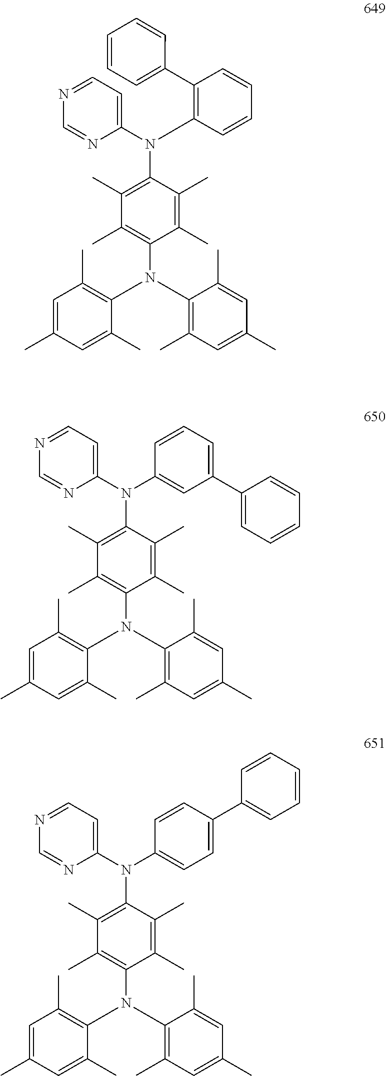 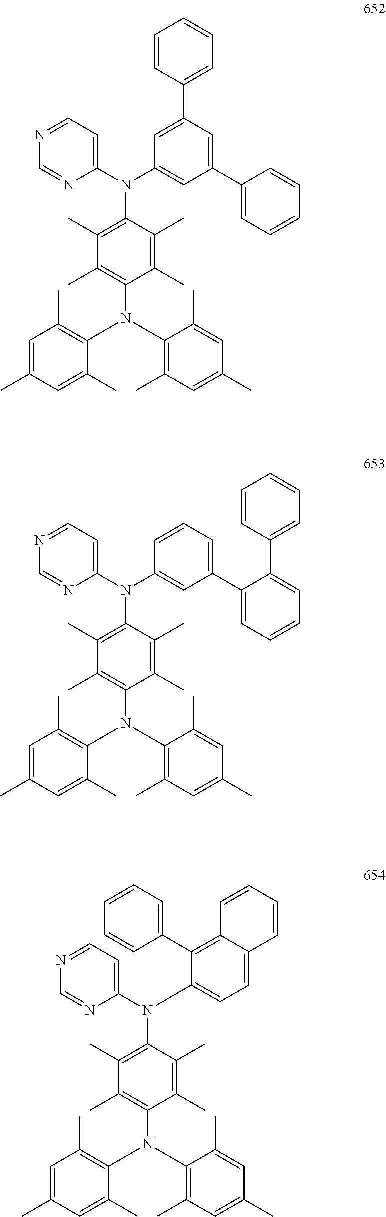 [SMILES: Cc1cc(C)c(N(c2c(C)cc(C)cc2C)c2c(C)c(C)c(N(c3cc(-c4ccccc4)cc(-c4ccccc4)c3)c3ccncn3)c(C)c2C)c(C)c1 ; Cc1cc(C)c(N(c2c(C)cc(C)cc2C)c2c(C)c(C)c(N(c3cccc(-c4ccccc4-c4ccccc4)c3)c3ccncn3)c(C)c2C)c(C)c1 ; Cc1cc(C)c(N(c2c(C)cc(C)cc2C)c2c(C)c(C)c(N(c3ccncn3)c3ccc4ccccc4c3-c3ccccc3)c(C)c2C)c(C)c1] 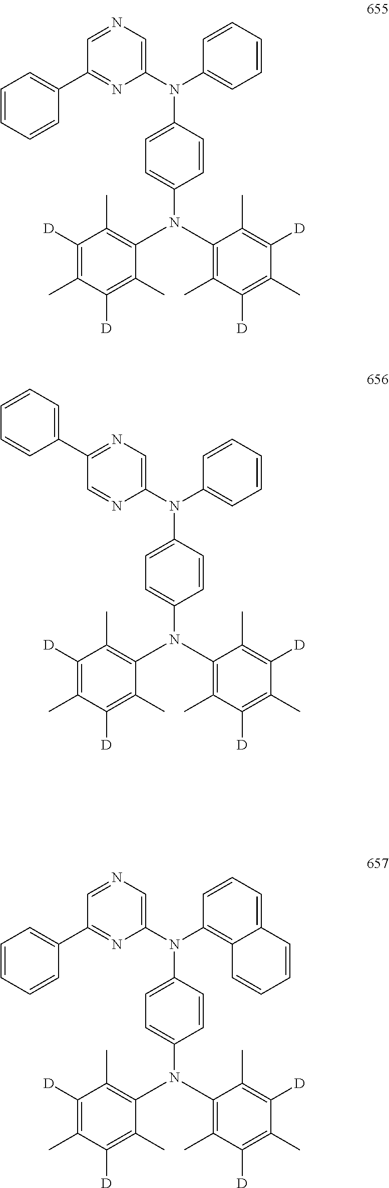 [SMILES: [2H]c1c(C)c([2H])c(C)c(N(c2ccc(N(c3ccccc3)c3cncc(-c4ccccc4)n3)cc2)c2c(C)c([2H])c(C)c([2H])c2C)c1C ; [2H]c1c(C)c([2H])c(C)c(N(c2ccc(N(c3ccccc3)c3cnc(-c4ccccc4)cn3)cc2)c2c(C)c([2H])c(C)c([2H])c2C)c1C ; [2H]c1c(C)c([2H])c(C)c(N(c2ccc(N(c3cncc(-c4ccccc4)n3)c3cccc4ccccc34)cc2)c2c(C)c([2H])c(C)c([2H])c2C)c1C] 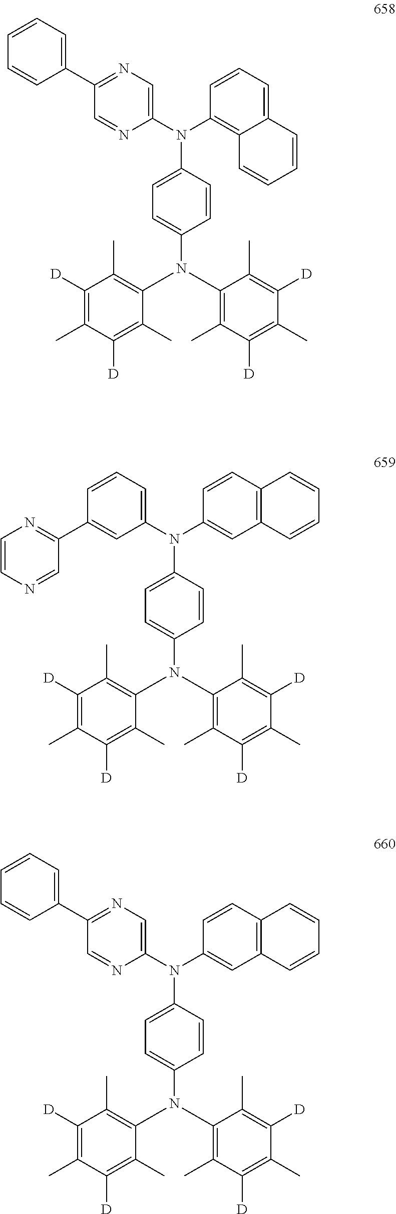 [SMILES: [2H]c1c(C)c([2H])c(C)c(N(c2ccc(N(c3cnc(-c4ccccc4)cn3)c3cccc4ccccc34)cc2)c2c(C)c([2H])c(C)c([2H])c2C)c1C ; [2H]c1c(C)c([2H])c(C)c(N(c2ccc(N(c3cccc(-c4cnccn4)c3)c3ccc4ccccc4c3)cc2)c2c(C)c([2H])c(C)c([2H])c2C)c1C ; [2H]c1c(C)c([2H])c(C)c(N(c2ccc(N(c3ccc4ccccc4c3)c3cnc(-c4ccccc4)cn3)cc2)c2c(C)c([2H])c(C)c([2H])c2C)c1C] 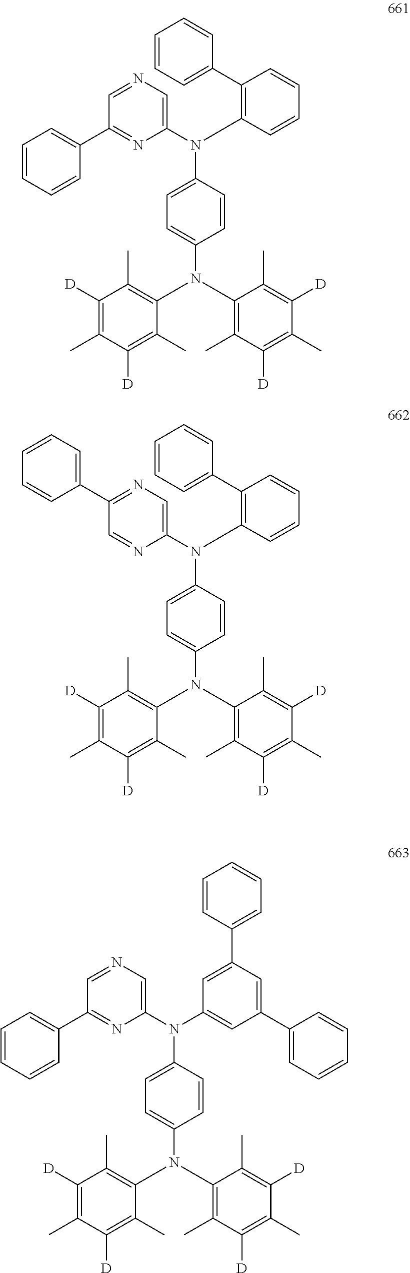 [SMILES: [2H]c1c(C)c([2H])c(C)c(N(c2ccc(N(c3cncc(-c4ccccc4)n3)c3ccccc3-c3ccccc3)cc2)c2c(C)c([2H])c(C)c([2H])c2C)c1C ; [2H]c1c(C)c([2H])c(C)c(N(c2ccc(N(c3cnc(-c4ccccc4)cn3)c3ccccc3-c3ccccc3)cc2)c2c(C)c([2H])c(C)c([2H])c2C)c1C ; [2H]c1c(C)c([2H])c(C)c(N(c2ccc(N(c3cc(-c4ccccc4)cc(-c4ccccc4)c3)c3cncc(-c4ccccc4)n3)cc2)c2c(C)c([2H])c(C)c([2H])c2C)c1C] 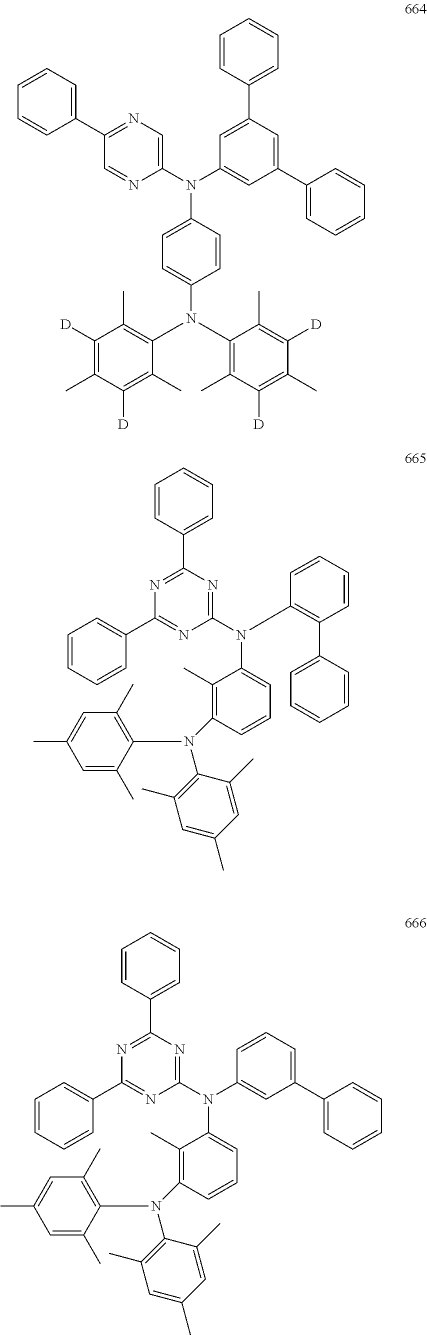 [SMILES: [2H]c1c(C)c([2H])c(C)c(N(c2ccc(N(c3cc(-c4ccccc4)cc(-c4ccccc4)c3)c3cnc(-c4ccccc4)cn3)cc2)c2c(C)c([2H])c(C)c([2H])c2C)c1C ; Cc1cc(C)c(N(c2cccc(N(c3nc(-c4ccccc4)nc(-c4ccccc4)n3)c3ccccc3-c3ccccc3)c2C)c2c(C)cc(C)cc2C)c(C)c1 ; Cc1cc(C)c(N(c2cccc(N(c3cccc(-c4ccccc4)c3)c3nc(-c4ccccc4)nc(-c4ccccc4)n3)c2C)c2c(C)cc(C)cc2C)c(C)c1] 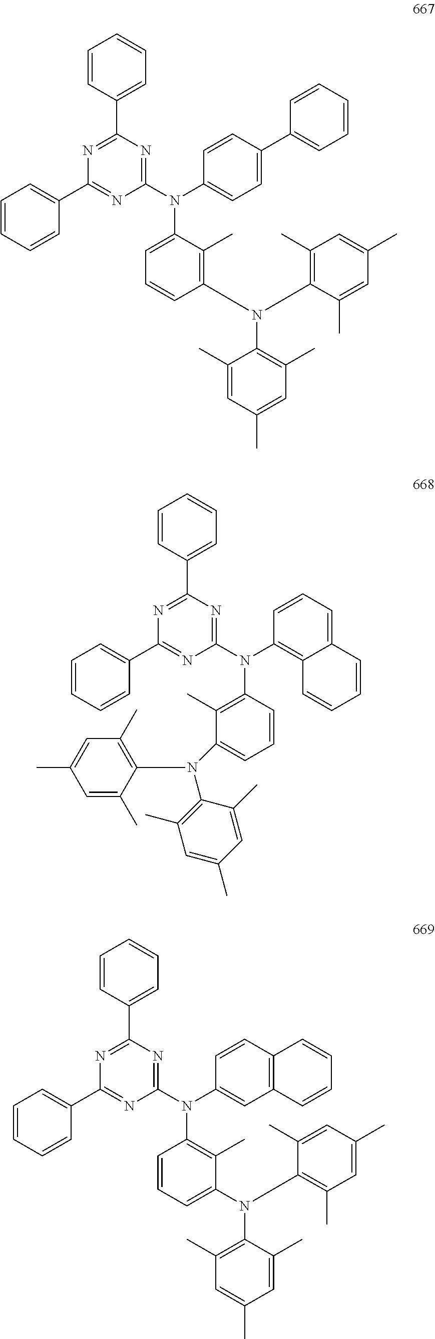 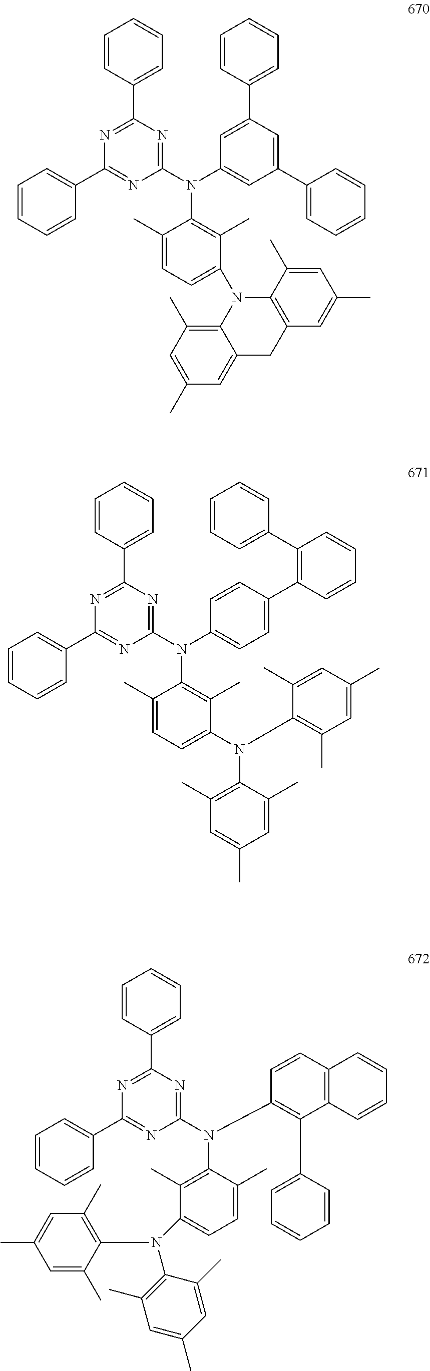 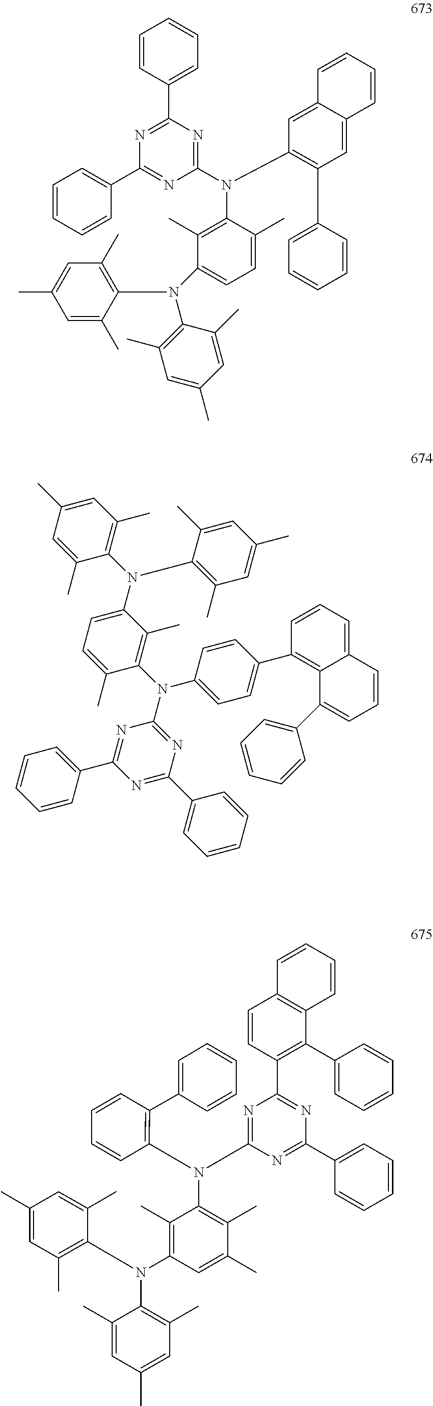 [SMILES: Cc1cc(C)c(N(c2ccc(C)c(N(c3nc(-c4ccccc4)nc(-c4ccccc4)n3)c3cc4ccccc4cc3-c3ccccc3)c2C)c2c(C)cc(C)cc2C)c(C)c1 ; Cc1cc(C)c(N(c2ccc(C)c(N(c3ccc(-c4cccc5cccc(-c6ccccc6)c45)cc3)c3nc(-c4ccccc4)nc(-c4ccccc4)n3)c2C)c2c(C)cc(C)cc2C)c(C)c1 ; Cc1cc(C)c(N(c2cc(C)c(C)c(N(c3nc(-c4ccccc4)nc(-c4ccc5ccccc5c4-c4ccccc4)n3)c3ccccc3-c3ccccc3)c2C)c2c(C)cc(C)cc2C)c(C)c1] 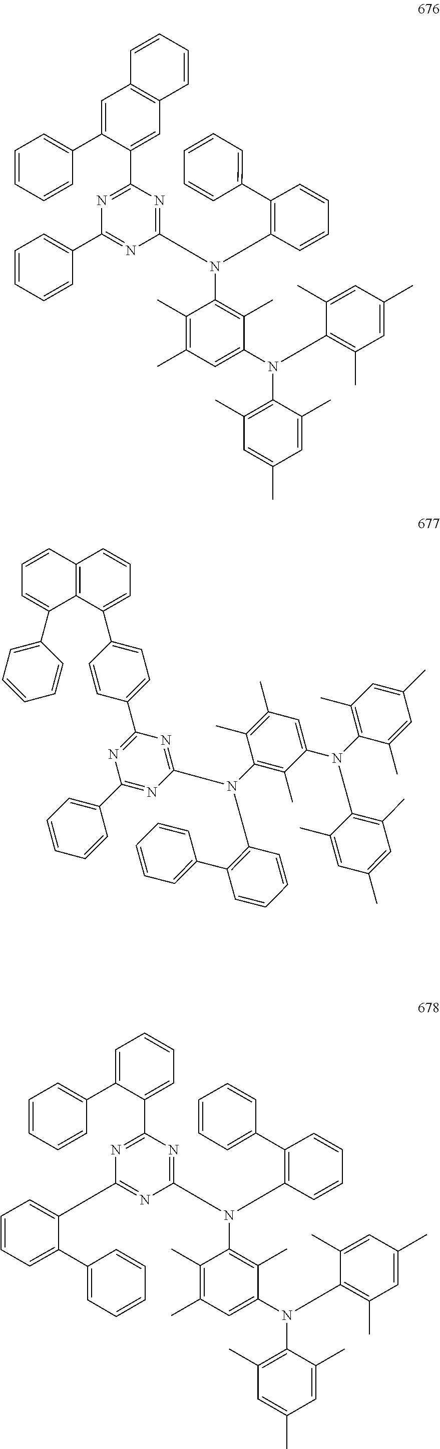 [SMILES: Cc1cc(C)c(N(c2cc(C)c(C)c(N(c3nc(-c4ccccc4)nc(-c4cc5ccccc5cc4-c4ccccc4)n3)c3ccccc3-c3ccccc3)c2C)c2c(C)cc(C)cc2C)c(C)c1 ; Cc1cc(C)c(N(c2cc(C)c(C)c(N(c3nc(-c4ccccc4)nc(-c4ccc(-c5cccc6cccc(-c7ccccc7)c56)cc4)n3)c3ccccc3-c3ccccc3)c2C)c2c(C)cc(C)cc2C)c(C)c1 ; Cc1cc(C)c(N(c2cc(C)c(C)c(N(c3nc(-c4ccccc4-c4ccccc4)nc(-c4ccccc4-c4ccccc4)n3)c3ccccc3-c3ccccc3)c2C)c2c(C)cc(C)cc2C)c(C)c1] 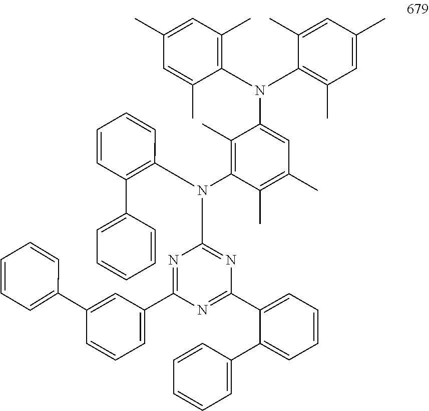 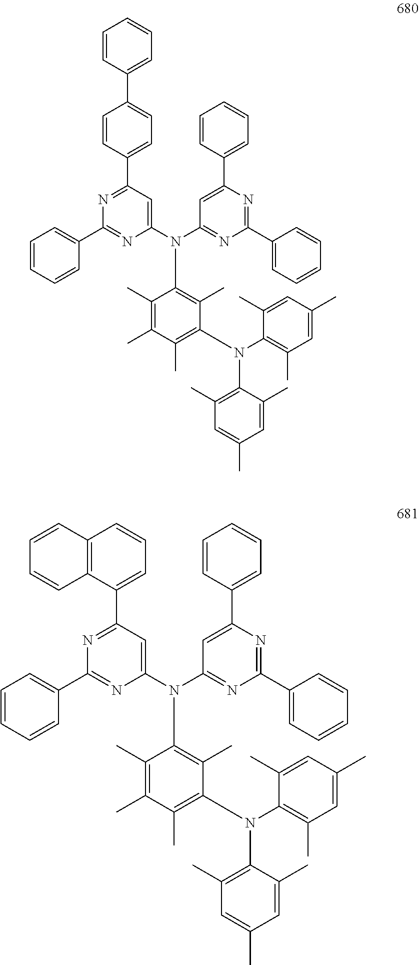 [SMILES: Cc1cc(C)c(N(c2c(C)cc(C)cc2C)c2c(C)c(C)c(C)c(N(c3cc(-c4ccccc4)nc(-c4ccccc4)n3)c3cc(-c4ccc(-c5ccccc5)cc4)nc(-c4ccccc4)n3)c2C)c(C)c1 ; Cc1cc(C)c(N(c2c(C)cc(C)cc2C)c2c(C)c(C)c(C)c(N(c3cc(-c4ccccc4)nc(-c4ccccc4)n3)c3cc(-c4cccc5ccccc45)nc(-c4ccccc4)n3)c2C)c(C)c1] 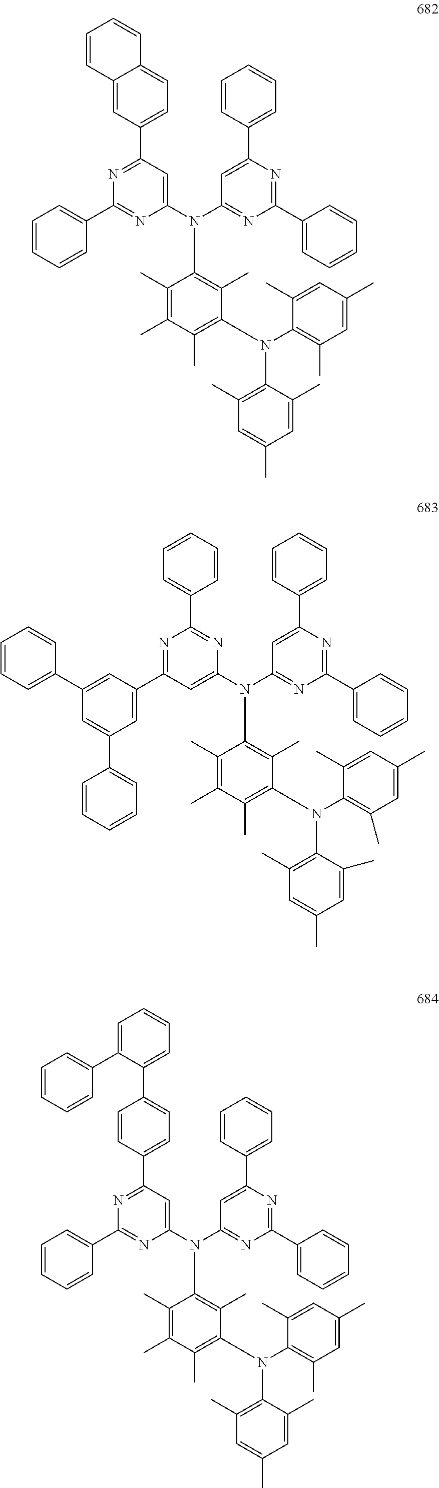 [SMILES: Cc1cc(C)c(N(c2c(C)cc(C)cc2C)c2c(C)c(C)c(C)c(N(c3cc(-c4ccccc4)nc(-c4ccccc4)n3)c3cc(-c4ccc5ccccc5c4)nc(-c4ccccc4)n3)c2C)c(C)c1 ; Cc1cc(C)c(N(c2c(C)cc(C)cc2C)c2c(C)c(C)c(C)c(N(c3cc(-c4ccccc4)nc(-c4ccccc4)n3)c3cc(-c4cc(-c5ccccc5)cc(-c5ccccc5)c4)nc(-c4ccccc4)n3)c2C)c(C)c1 ; Cc1cc(C)c(N(c2c(C)cc(C)cc2C)c2c(C)c(C)c(C)c(N(c3cc(-c4ccccc4)nc(-c4ccccc4)n3)c3cc(-c4ccc(-c5ccccc5-c5ccccc5)cc4)nc(-c4ccccc4)n3)c2C)c(C)c1] 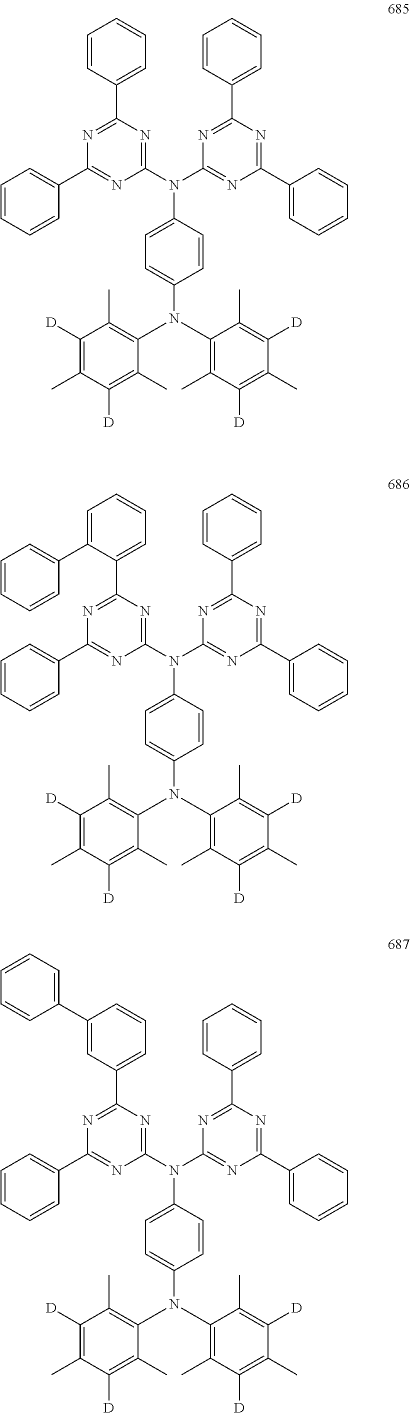 [SMILES: [2H]c1c(C)c([2H])c(C)c(N(c2ccc(N(c3nc(-c4ccccc4)nc(-c4ccccc4)n3)c3nc(-c4ccccc4)nc(-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4cccc(-c5ccccc5)c4)n3)cc2)c2c(C)c([2H])c(C)c([2H])c2C)c1C] 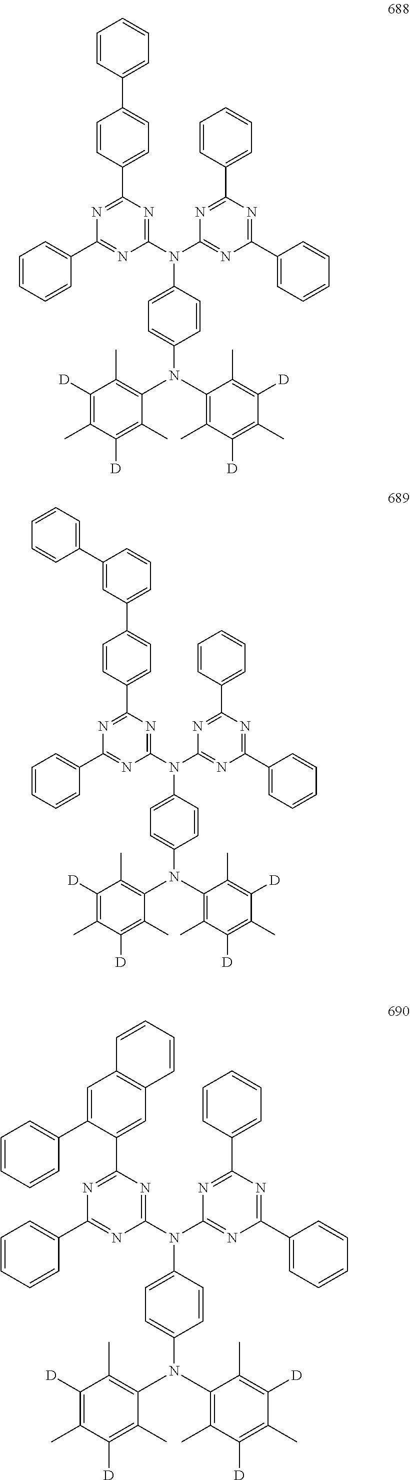 [SMILES: [2H]c1c(C)c([2H])c(C)c(N(c2ccc(N(c3nc(-c4ccccc4)nc(-c4ccccc4)n3)c3nc(-c4ccccc4)nc(-c4ccc(-c5ccccc5)cc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4ccc(-c5cccc(-c6ccccc6)c5)cc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4)n3)c3nc(-c4ccccc4)nc(-c4cc5ccccc5cc4-c4ccccc4)n3)cc2)c2c(C)c([2H])c(C)c([2H])c2C)c1C] 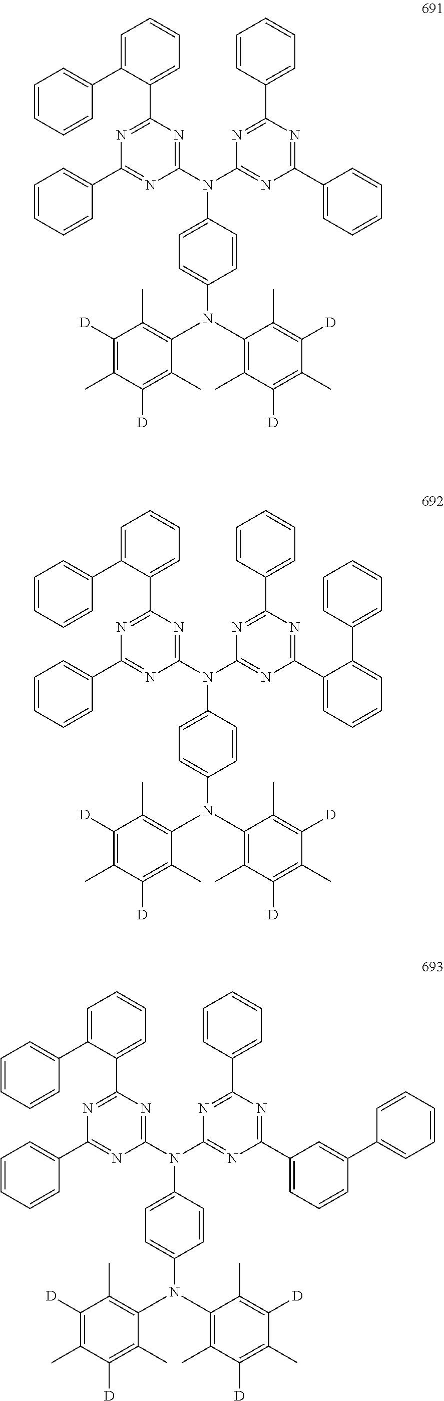 [SMILES: [2H]c1c(C)c([2H])c(C)c(N(c2ccc(N(c3nc(-c4ccccc4)nc(-c4ccccc4)n3)c3nc(-c4ccccc4)nc(-c4ccccc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4ccccc4-c4ccccc4)n3)c3nc(-c4ccccc4)nc(-c4ccccc4-c4ccccc4)n3)cc2)c2c(C)c([2H])c(C)c([2H])c2C)c1C ; [2H]c1c(C)c([2H])c(C)c(N(c2ccc(N(c3nc(-c4ccccc4)nc(-c4cccc(-c5ccccc5)c4)n3)c3nc(-c4ccccc4)nc(-c4ccccc4-c4ccccc4)n3)cc2)c2c(C)c([2H])c(C)c([2H])c2C)c1C] 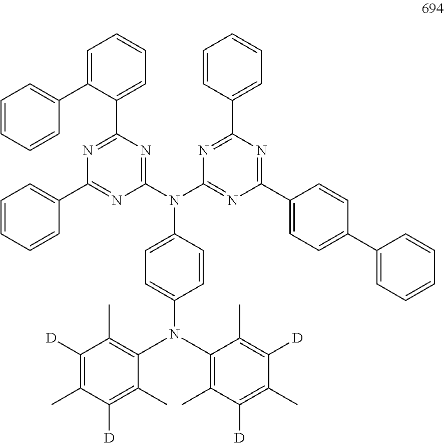 [SMILES: [2H]c1c(C)c([2H])c(C)c(N(c2ccc(N(c3nc(-c4ccccc4)nc(-c4ccc(-c5ccccc5)cc4)n3)c3nc(-c4ccccc4)nc(-c4ccccc4-c4ccccc4)n3)cc2)c2c(C)c([2H])c(C)c([2H])c2C)c1C]
